(19) United States
(12) Patent Application Publication
(10) Pub. No.: US 2008/0119457 A1
(43) Pub. Date: May 22, 2008

(54) Benzene, Pyridine, and Pyridazine Derivatives

(75) Inventors: Kenneth He Huang, Chapel Hill, NC (US); John Mangette, Clifton Park, NY (US); Thomas Barta, Carrboro, NC (US); Steven E. Hall, Chapel Hill, NC (US); James Veal, Apex, NC (US); Philip Hughes, Chapel Hill, NC (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: SERENEX, INC.

(21) Appl. No.: 11/844,816

(22) Filed: Aug. 24, 2007

Related U.S. Application Data
(60) Provisional application No. 60/823,414, filed on Aug. 24, 2006.

(51) Int. Cl. A61K 31/55; A61K 31/551; A61K 31/5377; A61K 31/506; A61K 31/496; A61K 31/501; C07D 413/02; C07D 403/02

(52) U.S. Cl. 514/217.04; 514/217.05; 514/235.5; 514/218; 514/252.02; 514/252.14; 514/253.01; 544/122; 544/295; 544/360; 540/575; 540/597; 540/600

(57) ABSTRACT

Disclosed are compounds and pharmaceutically acceptable salts of Formula I

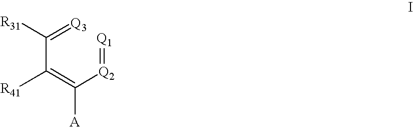

wherein A, Q₁, Q₂, Q₃, R₃₁, and R₄₁ are as defined herein. Compounds of Formula I are useful in the treatment of diseases and/or conditions related to cell proliferation, such as cancer, inflammation, arthritis, angiogenesis, or the like. Also disclosed are pharmaceutical compositions comprising compounds of the invention and methods of treating the aforementioned conditions using such compounds.

This application claims the benefit of Provisional Application No. 60/823,414, filed Aug. 24, 2006, the disclosure of which in incorporated herein in its entirety.

### BACKGROUND OF THE INVENTION

1. Field of the Invention

The invention relates to benzene, pyridine, and pyridazine derivatives and more specifically to such compounds that are useful in the treatment and/or prevention of diseases and/or conditions related to cell proliferation, such as cancer, inflammation and inflammation-associated disorders, and conditions associated with angiogenesis. Compounds of the invention are also useful in the treatment and/or prevention of infectious diseases, in particular, fungal, and viral infections.

2. Description of the Related Art

Cancer is characterized by abnormal cellular proliferation. Cancer cells exhibit a number of properties that make them dangerous to the host, typically including an ability to invade other tissues and to induce capillary ingrowth, which assures that the proliferating cancer cells have an adequate supply of blood. A hallmark of cancerous cells is their abnormal response to control mechanisms that regulate cell division in normal cells and continue to divide until they ultimately kill the host.

Angiogenesis is a highly regulated process under normal conditions, however many diseases are driven by persistent unregulated angiogenesis. Unregulated angiogenesis may either cause a particular disease directly or exacerbate an existing pathological condition. For example, ocular neovascularization has not only been implicated as the most common cause of blindness, but also is believed the dominant cause of many eye diseases. Further, in certain existing conditions, for example arthritis, newly formed capillary blood vessels invade the joints and destroy cartilage, or in the case of diabetes, new capillaries formed in the retina invade the vitreous, bleed, and cause blindness. Growth and metastasis of solid tumors are also dependent on angiogenesis (Folkman, J., Cancer Research, 46, 467-473 (1986), Folkman, J., Journal of the National Cancer Institute, 82, 4-6 (1989). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites such as liver, lung or bone (Weidner, N., et al., The New England Journal of Medicine, 324(1), 1-8 (1991). Under conditions of unregulated angiogenesis, therapeutic methods designed to control, repress, and/or inhibit angiogenesis could lead to the abrogation or mitigation of these conditions and diseases.

Inflammation is related to a variety of disorders such as pain, headaches, fever, arthritis, asthma, bronchitis, menstrual cramps, tendonitis, bursitis, psoriasis, eczema, burns, dermatitis, inflammatory bowel syndrome, Crohn's disease, gastritis, irritable bowel syndrome, ulcerative colitis, vascular diseases, Hodgkin's disease, scleroderma, rheumatic fever, type I diabetes, myasthenia gravis, sarcoidosis, nephrotic syndrome, Behcet's syndrome, polymyositis, hypersensitivity, conjunctivitis, gingivitis, post-injury swelling, myocardial ischemia, cerebral ischemia (stroke), sepsis and the like.

Heat-shock protein 90 (HSP-90) is a cellular chaperone protein required for the activation of several eukaryotic protein kinases, including the cyclin-dependent kinase CDK4. Geldanamycin, an inhibitor of the protein-refolding activity of HSP-90, has been shown to have antiproliferative and antitumor activities.

HSP-90 is a molecular chaperone that guides the normal folding, intracellular disposition and proteolytic turnover of many key regulators of cell growth and survival. Its function is subverted during oncogenesis to make malignant transformation possible and to facilitate rapid somatic evolution, and to allow mutant proteins to retain or even gain function. Inhibition of HSP-90 will slow those process thus has potential therapeutic use (Whitesell L, Lindquist, S L, Nature Rev. Cancer, 2005, 10, 761-72).

Ansamycin antibiotics, e.g., herbimycin A (HA), geldanamycin (GM), and 17-allylaminogeldanamycin (17-AAG) are thought to exert their anticancerous effects by tight binding of the N-terminus pocket of HSP-90, thereby destabilizing substrates that normally interact with HSP-90 (Stebbins, C. et al. Cell 1997, 89, 239-250). This pocket is highly conserved and has weak homology to the ATP-binding site of DNA gyrase (Stebbins, C. et al., supra; Grenert, J. P. et al. J. Biol. Chem. 1997, 272,23843-50).

In vitro and in vivo studies have demonstrated that occupancy of this N-terminal pocket by ansamycins and other HSP-90 inhibitors alters HSP-90 function and inhibits protein folding. At high concentrations, ansamycins and other HSP-90 inhibitors have been shown to prevent binding of protein substrates to HSP-90 (Scheibel, T. H. et al. Proc. Natl. Acad. Sci. USA 1999, 96, 1297-302; Schulte, T. W. et al. J. Biol. Chem. 1995, 270,24585-8; Whitesell, L., et al. Proc. Natl. Acad. Sci. USA 1994, 91, 8324-8328). Ansamycins have also been demonstrated to inhibit the ATP-dependent release of chaperone-associated protein substrates (Schneider, C. L. et al. Proc. Natl. Acad. Sci., USA 1996, 93, 14536-41; Sepp-Lorenzino et al. J. Biol. Chem. 1995, 270,16580-16587). In either event, the substrates are degraded by a ubiquitin-dependent process in the proteasome (Schneider, C. L., supra; Sepp-Lorenzino, L., et al. J. Biol. Chem. 1995, 270, 16580-16587; Whitesell, L. et al. Proc. Natl. Acad. Sci. USA 1994, 91, 8324-8328). HSP-90 substrate destabilization occurs in tumor and non-transformed cells alike and has been shown to be especially effective on a subset of signaling regulators, e.g., Raf (Schulte, T. W. et al., Biochem. Biophys. Res. Commun. 1997, 239, 655-9 Schulte, T. W., et al., J. Biol. Chem. 1995, 270, 24585-8), nuclear steroid receptors (Segnitz, B.; U. Gehring J. Biol. Chem. 1997, 272, 18694-18701; Smith, D. F. et al. Mol. Cell. Biol. 1995, 15, 6804-12), v-Src (Whitesell, L., et al. Proc. Natl. Acad. Sci. USA 1994, 91, 8324-8328) and certain transmembrane tyrosine kinases (Sepp-Lorenzino, L. et al. J. Biol. Chez. 1995, 270, 16580-16587) such as EGF receptor (EGFR) and HER2/Neu (Hartmann, F., et al. Int. J. Cancer 1997, 70, 221-9; Miller, P. et al. Cancer Res. 1994, 54, 2724-2730; Mimnaugh, E. G., et al. J. Biol. Clzem. 1996, 271, 22796-801; Schnur, R. et al. J. Med. Chenu. 1995, 38, 3806-3812), CDK4, and mutant p53. Erlichman et al. Proc. AACR 2001, 42, abstract 4474. The ansamycin-induced loss of these proteins leads to the selective disruption of certain regulatory pathways and results in growth arrest at specific phases of the cell cycle (Muise-Heimericks, R. C. et al. J. Biol. Chez. 1998, 273, 29864-72), and apoptosis, and/or differentiation of cells so treated (Vasilevskaya, A. et al. Cancer Res., 1999, 59, 3935-40). Inhibitors of HSP-90 thus hold great promise for the treatment and/or prevention of many types of cancers and proliferative disorders, and also hold promise as traditional antibiotics.

Inhibition of HSP-90 is also known to result in up regulation of the expression of the chaperone HSP70. HSP70 up regulation is considered to be of therapeutic benefit for treatment of a wide range of neurodegenerative diseases including, but not limited to: Alzheimer's disease; Parkinson's disease; Dementia with Lewy bodies; Amyotropic lateral scleriosis (ALS); Polyglutamine disease; Huntington's disease; Spinal and bulbar muscular atrophy (SBMA); and Spinocerebellar ataxias (SCA1-3,7). Therefore, the compounds described in the invention are of potential therapeutic use for treatment of such neurodegenerative diseases (Muchowski, P. J., Wacker J. L., Nat. Rev. Neurosci. 2005, 6, 11-22.; Shen H. Y., et al. J. Biol. Chem. 2005, 280, 39962-9).

Inhibition of HSP-90 also has anti-fungal activity, both as a stand alone therapy and in combination with standard anti-fungal therapies such as the azole class of drugs. Therefore, the compounds described in the invention are of potential therapeutic use for treatment of fungal infections including, but not limited to, life threatening systemic fungal infections (Cowen, L. E., Lindquist, S., Science 2005, 309, 2185-9).

Inhibition of HSP-90 is also expected to result in antimalarial activity; thus, inhibitors of this protein are useful as antimalarial drugs.

HSP-90 has also been shown to be important to viral transcription and replication, in particular for such processes in HIV-1 and Hepatitis C virus. See J Biol. Chem. 2000 Jan. 7; 275(1):279-87; J Virol. 2004 December; 78(23):13122-31; and Biochem Biophys Res Commun. 2007 Feb. 23; 353(4):882-8. Epub 2006 Dec. 22.

Inhibitors of HSP-90 have been shown to attenuate inflammation via lowering the level of a number of client proteins associated inflammation process. See FASEB J. 2007 July; 21(9):2113-23. Therefore, there is a continuing need in the art for new methods of treating cancer, inflammation and inflammation-associated disorders, and conditions or diseases related to uncontrolled angiogenesis.

### SUMMARY OF THE INVENTION

In a broad aspect, the invention encompasses compounds of formula I,

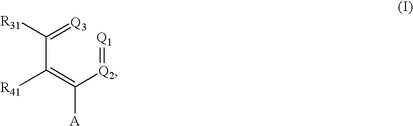

wherein A, Q₁, Q₂, Q₃, R₃₁, and R₄₁ are defined herein, pharmaceutical compositions containing those compounds and methods employing such compounds or compositions in the treatment of diseases and/or conditions related to cell proliferation, such as cancer, inflammation, arthritis, angiogenesis, or the like.

The invention also includes intermediates that are useful in making the compounds of the invention.

The invention also provides pharmaceutical compositions comprising a compound or pharmaceutically acceptable salt of Formula I and at least one pharmaceutically acceptable carrier, solvent, adjuvant or diluent.

The invention further provides methods of treating disease such as cancer, inflammation, arthritis, angiogenesis, and infection in a patient in need of such treatment, comprising administering to the patient a compound or pharmaceutically acceptable salt of Formula I, or a pharmaceutical composition comprising a compound or salt of Formula I.

The invention also provides methods of treating and/or preventing viral infections in patients in need of such treatment comprising administration of a compound or salt of formula I.

The invention also provides the use of a compound or salt according to Formula I for the manufacture of a medicament for use in treating cancer, inflammation, arthritis, angiogenesis, or infection.

The invention also provides methods of preparing the compounds of the invention and the intermediates used in those methods.

The invention also provides methods of treating a disease or condition related to cell proliferation comprising administering a therapeutically effective amount of a compound or salt of Formula I to a patient in need of such treatment.

The invention also provides methods of treating a disease or condition related to cell proliferation comprising administering a therapeutically effective amount of a compound or salt of Formula I to a patient in need of such treatment, where the disease of condition is cancer, inflammation, or arthritis.

The invention further provides methods of treating a subject suffering from a disease or disorder of proteins that are either client proteins for HSP-90 or indirectly affect its client proteins, comprising administering to a subject in need of such treatment a therapeutically effective amount of a compound or salt of Formula I.

The invention further provides methods of treating a subject suffering from a disease or disorder of proteins that are either client proteins for HSP-90 or indirectly affect its client proteins, comprising administering to a subject in need of such treatment a therapeutically effective amount of a compound or salt of Formula I, wherein the HSP-90 mediated disorder is selected from the group of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases and malignant disease.

The invention further provides methods of treating a subject suffering from a fibrogenetic disorder of proteins that are either client proteins for HSP-90 or indirectly affect its client proteins, comprising administering to a subject in need of such treatment a therapeutically effective amount of a compound or salt of Formula I, wherein the fibrogenetic disorder is selected from the group of scleroderma, polymyositis, systemic lupus, rheumatoid arthritis, liver cirrhosis, keloid formation, interstitial nephritis and pulmonary fibrosis.

The invention provides methods of protecting a subject from infection caused by an organism selected from Plasmodium species, preferably Plasmodium falciparum. These methods comprising administering a compound or salt of Formula I, preferably in an effective amount, to a subject at risk of infection due to exposure to such organism.

The invention additionally provides methods of reducing the level of infection in a subject where the infection is caused by an organism selected from Plasmodium species, again preferably Plasmodium falciparum. These methods comprise administering to an infected subject an effective amount of a compound or salt of Formula I.

The invention further provides methods for treating a patient infected with a metazoan parasite. These methods involve administering an amount of a compound of the invention effective to kill the parasite.

The invention further provides methods for treating a patient infected with a metazoan parasite wherein the parasite is Plasmodium falciparum. These methods involve administering an amount of a compound or salt of the invention effective to kill the parasite.

The invention further encompasses kits comprising compounds of the invention or pharmaceutical compositions thereof in a package with instructions for using the compound or composition.

The invention further provides compounds that may be administered alone or in combination with other drugs or therapies known to be effective to treat the disease to enhance overall effectiveness of therapy.

The invention further provides methods for treating a fungal infection in a patient in need of such treatment, comprising administering an effective amount of a compound or salt of Formula I and an optional anti-fungal agent or drug.

### DETAILED DESCRIPTION OF THE INVENTION

The invention provides compounds of formula I,

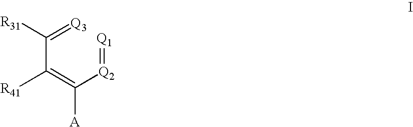

or a pharmaceutically acceptable salt thereof, wherein
each m is independently 0, 1, or 2;
each R_{c} independently is halogen, cyano, nitro, or —R_{N};
each R_{N} is independently —R_{N′}, —C(O)R_{N′}, —C(O)OR_{N′}, —C(O)N(R_{N′})₂, —S(O)R_{N′}, or —S(O)₂R_{N′} wherein
each R_{N′} is independently hydrogen, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl, wherein
   each R_{N′} is optionally substituted with from 1 to 4 R groups;
      each R_{O} is independently —R_{N′}, —C(O)R_{N′}, —C(O)OR_{N′}, or —C(O)N(R_{N′})₂;
      each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆)alkylamino, carboxy, carboxamide, C₃-C₇ cycloalkyl, heterocycloalkyl, aryl, or heteroaryl;

### Q₁, Q₂, and Q₃ are independently N or CR_{Q}, provided that no more than two of Q₁, Q₂, and Q₃ are not simultaneously N, wherein

each R_{Q} is independently hydrogen, halogen, —O(R_{O}), —N(R_{N})₂, C₁-C₆ alkyl, C₁-C₆ haloalkyl, C₃-C₇ cycloalkyl, aryl, or heteroaryl, or R₂₁, wherein
   each R_{Q} is optionally substituted with from 1 to 4 R groups; and
   R₂₁ is cyano, —C(O)OH, —C(O)—O(C₁-C₆alkyl), or —C(X)N(R₁₁₁)₂, wherein
      each R₁₁₁ is independently H, hydroxy, C₁-C₆ alkyl, C₂-C₆ alkenyl, C₂-C₆ alkynyl, heteroaryl, aryl, C₃-C₈ cycloalkyl, heterocycloalkyl,
         wherein each R₁₁₁ is optionally substituted with from 1 to 4 R groups;
      or both R₁₁₁ taken together with the nitrogen to which they are attached, form a heterocycloalkyl; and
      X is ═O, ═S, ═NH, ═NOH, ═N—NH₂, ═N—NH-aryl, ═N—═NH—(C₁-C₆ alkyl), or ═N—(C₁-C₆ alkoxy);

### A is one of the formulas (i), (ii), (iii), or (iv),

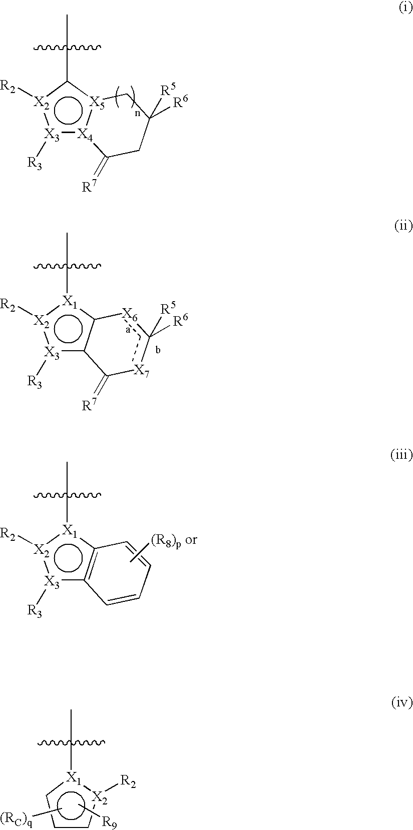

wherein
bonds a and b are independently a single or double bond, provided that
   (i) when a is double bond, then b is a single bond, X₆ is CH or N, X₇ is CH₂ or NR_{N}, and R₆ is absent;
   (ii) when b is double bond, then a is a single bond, X₆ is CH₂, O, S(O)ₘ, or NR_{N}, X₇ is CH or N, and R₆ is absent;
   (iii) when a and b are both single bonds, then X₆ is O, S(O)ₘ, or NR_{N}, and X₇ is CH₂, NR_{N}, or O; and
n is 0, 1, 2, 3, or 4;
p is 1, 2, 3, or 4;
q is 0, 1, or 2;
X₁ is C or N;
X₂ and X₃ are independently C, N, O, or S;
X₄ and X₅ are independently C or N;
provided that
   (i) for only formula (I),
      (a) either exactly one of X₂, X₃, X₄, and X₅ is N, O, or S and the remaining three are C; or exactly two of X₂, X₃, X₄, and X₅ are N, and the remaining two are C; and
      (b) X₄ and X₅ cannot be O or S;
   (ii) for only formulas (II) and (iii),
      (a) either exactly one of X₁, X₂, and X₃ is N, O, or S and the remaining two are C; or exactly two of X₁, X₂, and X₃ are N, and the remaining one is C; and
      (b) X₁ cannot be S or O; and
   (iii) for formula (iv), X₁ cannot be O or S;
provided that for all of formulas (i), (ii), (iii), and (iv) each of R₂ and R₃ is absent when the atom to which they are connected is of insufficient valency to carry a substituent;
R₂ is R_{C} when X₂ is C; or R₂ is R_{N} when X₂ is N;
R₃ is R_{C} when X₃ is C; or R₃ is R_{N} when X₃ is N;
R₅ and R₆ are independently H, C₁-C₆ alkyl, or aryl, wherein the aryl is optionally substituted with from 1 to 4 R groups,
   wherein any two adjacent substituted aryl positions, together with the carbon atoms to which they are attached, optionally form an unsaturated cycloalkyl or heterocycloalkyl;
or R₅ and R₆ together with the carbon to which they are attached form a 3-8 membered ring;
R₇ is O, S, NH, N—OH, N—NH₂, N—NHR₂₂, N—NH—(C₁-C₆ alkyl), N—O— (CO—C₆)alkyl-R₂₂, or N—(C₁-C₆ alkoxy optionally substituted with carboxy);
each R₈ is independently —OR₈₁, —N(R₈₁)₂, or —R_{C},
   wherein each R₈₁ is independently —H, —R₂₂, C₁-C₆ alkyl, or halo(C₁-C₆)alkyl,
      wherein each R₈₁ is optionally substituted with 1-2 groups which are independently R_{C}, —OR_{O}, —SR_{O}, or —N(R_{N})₂;
R₉ is —C₁-C₆ alkoxy or a group of the formula,

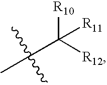

wherein
   R₁₀ is C₁-C₆ alkyl or halo(C₁-C₆)alkyl, either optionally substituted with 1 or 2 R₁₀₁ groups, wherein
      each R₁₀₁ is independently halogen, nitro, cyano, C₁-C₆ alkyl, halo(C₁-C₆)alkyl, —OR_{O}, —N(R_{N})₂, —S(O)ₘR_{N′}, or —C(O)R_{N′};
   R₁₁ is H; and
   R₁₂ is H or —OR_{O};
   or R₁₁ and R₁₂ together are R₇; and
each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, wherein
   each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
      each R₂₂ is optionally fused to a C₆-C₁₀ aryl group, C₅-C₈ saturated cyclic group, or a C₅-C₁₀ heterocycloalkyl group;
or when A is formula (iv), R₉ and R_{C} together with the atoms to which they are attached optionally form a 5-7 membered carbocyclic ring fused to the ring carrying X₁ and X₂, the 5-7 membered ring being fused adjacent to X₂, and where the 5-7 membered carbocyclic ring is optionally substituted with oxo and 1-3 of C₁-C₆ alkyl; and
R₃₁ and R₄₁ are independently (a) H, (b) halo, or (c) a C₁-C₁₅ alkyl group where up to six of the carbon atoms in said alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
   wherein each (c) is optionally substituted with —R_{C}, —OR₁₅, —SR₁₅, —N(R₁₅)₂, or —R₂₂, wherein
      each R₁₅ is independently —H, (C₁-C₁₀)alkyl, (C₁-C₁₀) haloalkyl, (C₂-C₆)alkenyl, (C₂-C₆)alkynyl, or (C₁-C₁₀)alkyl-Z, wherein
         Z is —OR_{O} or —N(R₃₀)₂, wherein
            each R₃₀ is independently —H or C₁-C₆ alkyl;
            or N(R₃₀)₂ represents pyrrolidinyl, piperidinyl, piperazinyl, azepanyl, 1,3- or 1,4-diazepanyl, or morpholinyl, each of which is optionally substituted with R;
               or R₃₁ and R₄₁ together with the atoms to which they are attached form a 5-12 membered mono-, bi-, or tricyclic ring system fused to the ring containing Q₁ and Q₂, where the 5-12 membered ring is partially unsaturated or aromatic and optionally contains one or two of oxygen, S(O)ₘ, nitrogen, or —NR₃₃ where R₃₃ is hydrogen or C₁-C₆ alkyl.

In Formula I, R₃₁ and R₄₁ are, as noted above, independently (a) hydrogen, (b) halo, or (c) an alkyl group having from 1-15 carbon atoms. All, but no more than about six, of the carbon atoms in the alkyl group may be replaced independently by the various groups listed above in connection with Formula I. Replacement of any carbon atom is permitted, i.e., both internal and terminal carbon atoms. Further, the alkyl groups of from 1-15 carbon atoms may be straight or branched.

Thus, when the alkyl group is methyl, i.e., a one carbon atom alkyl group, replacement of that carbon atom with, for example, nitrogen or sulfur, the resulting group will not be an alkyl group but instead will be an amino or thio group, respectively. Similarly, when the carbon atom being replaced terminates the alkyl group, the terminal group will become another moiety such as pyrimidinyl, amino, phenyl, or hydroxy.

Replacement of a carbon atom with a group such as, for example, oxygen, nitrogen, or sulfur will require appropriate adjustment of the number of hydrogens or other atoms required to satisfy the replacing atom's valency. Thus, when the replacement is N or O, the number of groups attached to the atom being replaced will be reduced by one or two to satisfy the valency of the nitrogen or oxygen respectively. Similar considerations will be readily apparent to those skilled in the art with respect to replacement by ethenyl and ethynyl.

Thus, replacement as permitted herein results in the term “C₁-C₁₅ alkyl” as defined in connection with Formula I encompassing groups such as, but not limited to:
amino, hydroxy, phenyl, benzyl, propylaminoethoxy, butoxyethylamino, pyrid-2-ylpropyl, diethylaminomethyl, pentylsulfonyl, methylsulfonamidoethyl, 3-[4-(butylpyrimidin-2-yl)ethyl]phenyl, butoxy, dimethylamino, 4-(2-(benzylamino)ethyl)pyridyl, but-2-enylamino, 4-(1-(methylamino)pent-3-en-2-ylthio)phenyl, 2-(N-methyl-hexanamido)ethoxy)methyl, and 4-(((3-methoxy-4-(4-methyl-1H-imidazol-2-yl)but-1-enyl)(methyl)amino)-methyl)phenyl.

Further, replacement as permitted herein may result in an R₃ group that exceeds 15 atoms. For example, replacing 6 carbon atoms of a 11-carbon atom straight chain alkyl group with amino, tetrahydropyran, amino, chlorophenyl, imidazolyl, and hydroxy could result in an R₃ group of the formula:

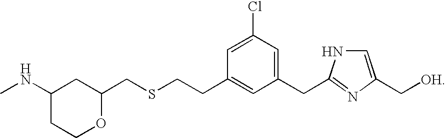

Preferred compounds of Formula I include those where R₃₁ and R₄₁ are independently hydrogen, halo, or -Z₁R_{Z1}, wherein Z₁ is —O—, —NH—, —S(O)ₘ—, or —S(O)₂NH—, and R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S(O)ₘ—(C₁-C₆)alkyl, —S(O)ₘ-aryl, —SO₂NH₂, —SO₂NH—(C₁-C₆)alkyl, —SO₂NH-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z₁ or R₂₂.

Even more preferred compounds of Formula I include those where R₃₁ and R₄₁ are independently hydrogen, halo, or -Z₁R_{Z1}, wherein Z₁ is —O— or —NH—; and R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S(O)ₘ—(C₁-C₆)alkyl, —S(O)ₘ-aryl, —SO₂NH₂, —SO₂NH—(C₁-C₆)alkyl, —SO₂NH-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₂.

Additional preferred compounds of Formula I include those where R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S(O)ₘ—(C₁-C₆)alkyl, —S(O)ₙ-aryl, —SO₂NH₂, —SO₂NH—(C₁-C₆)alkyl, —SO₂NH-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₂.

Most preferred compounds of Formula I include those where R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂.

In one embodiment, the invention provides compounds according to formula (I) wherein A is

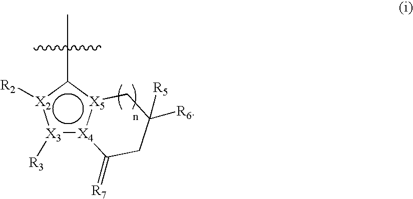

In a preferred embodiment, the invention provides compounds according to formula (I) wherein A is

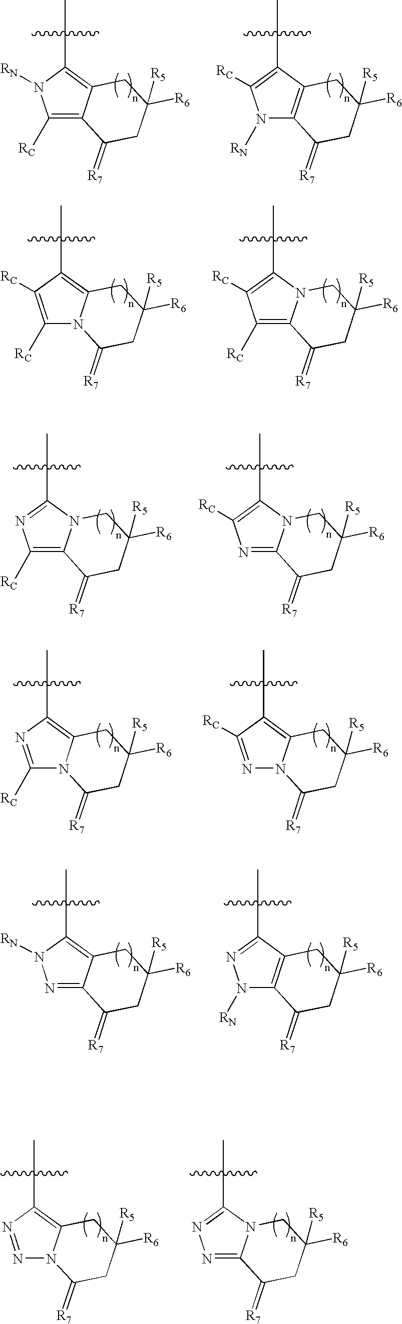

In a preferred embodiment, the invention provides compounds according to formula (I) wherein A is

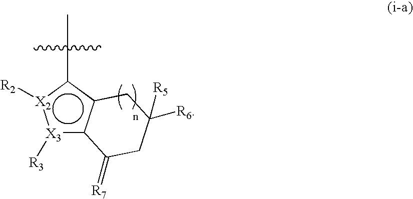

In another preferred embodiment, the invention provides compounds according to formula (I) wherein A is

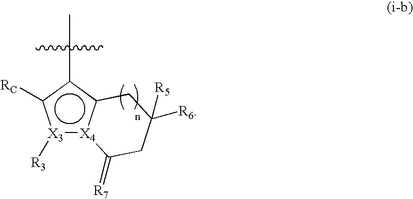

In another preferred embodiment, the invention provides compounds according to formula (I) wherein A is

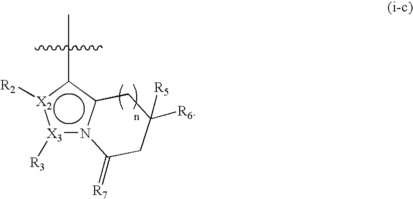

In another preferred embodiment, the invention provides compounds according to formula (I) wherein A is

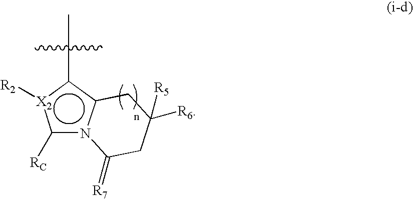

In another preferred embodiment, the invention provides compounds according to formula (I) wherein A is

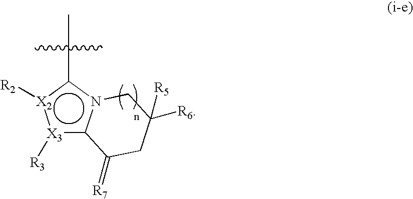

In another preferred embodiment, the invention provides compounds according to formula (I) wherein A is

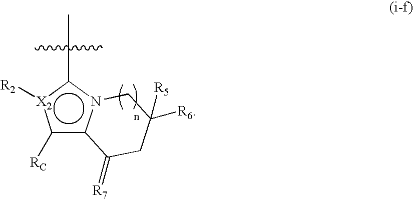

In one embodiment, the invention provides compounds according to formula (I) wherein A is

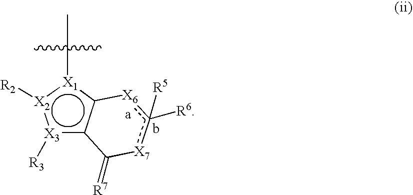

In a preferred embodiment, the invention provides compounds according to formula (I) wherein A is

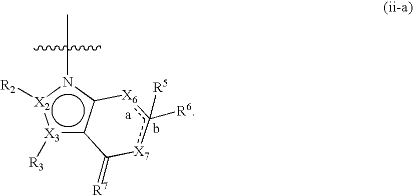

In more preferred embodiment, the invention provides compounds according to formula (I) wherein A is

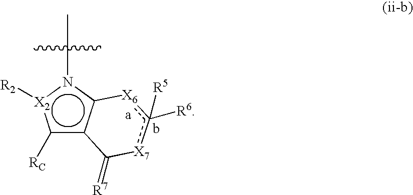

In another more preferred embodiment, the invention provides compounds according to formula (I) wherein A is

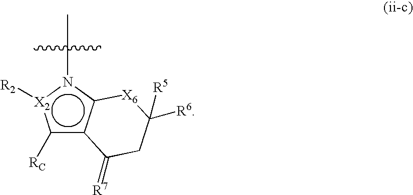

In another more preferred embodiment, the invention provides compounds according to formula (I) wherein A is

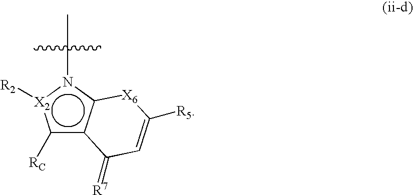

In another more preferred embodiment, the invention provides compounds according to formula (I) wherein A is

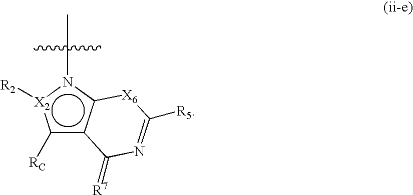

In one embodiment, the invention provides compounds according to formula (I) wherein A is

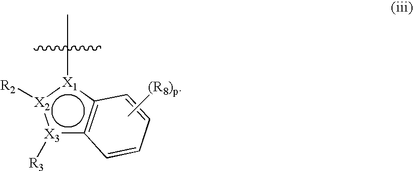

In a preferred embodiment, the invention provides compounds according to formula (I) wherein A is

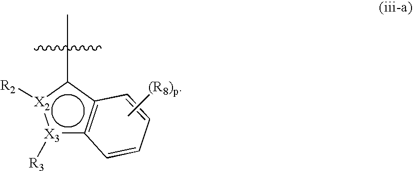

In a another preferred embodiment, the invention provides compounds according to formula (I) wherein A is

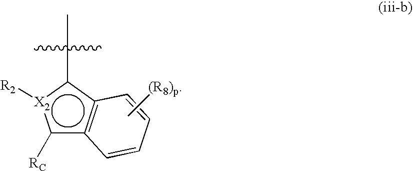

In a another preferred embodiment, the invention provides compounds according to formula (I) wherein A is

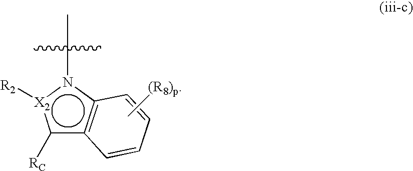

Preferred compounds of the invention where A is a group of formula iii-a, iii-b, or iii-c include those where p is 1, or 2, more preferably 1, and R₈ is C₁-C₆ alkoxy, more preferably methoxy or ethoxy.

In one embodiment, the invention provides compounds according to formula (I) wherein A is

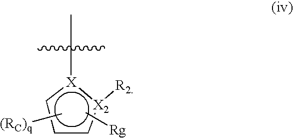

In a preferred embodiment, the invention provides compounds according to formula (I) wherein A is

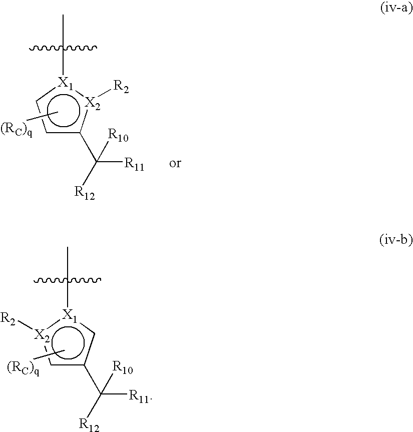

In a preferred embodiment, the invention provides compounds according to formula (I) wherein A is

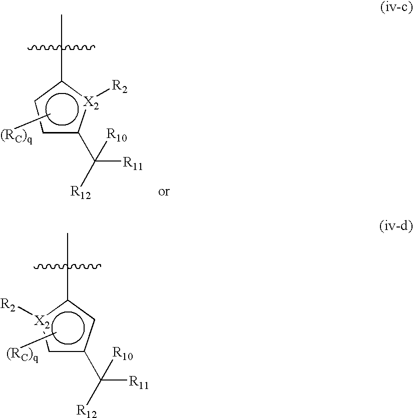

In a another preferred embodiment, the invention provides compounds according to formula (I) wherein A is

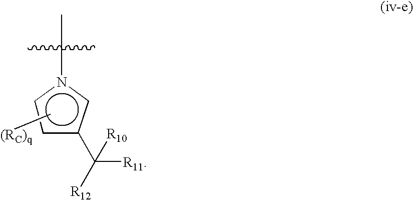

In one embodiment, the invention provides compounds according to formula (I) wherein A is

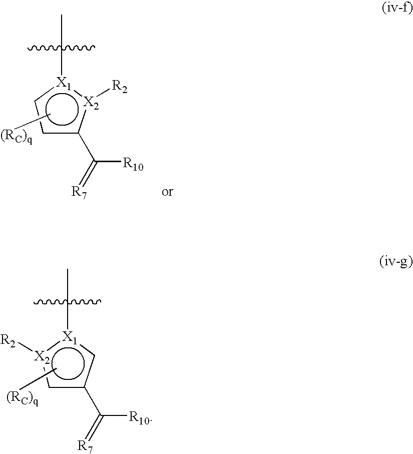

In one embodiment, the invention provides compounds according to formula (I) wherein A is one of the following structures,

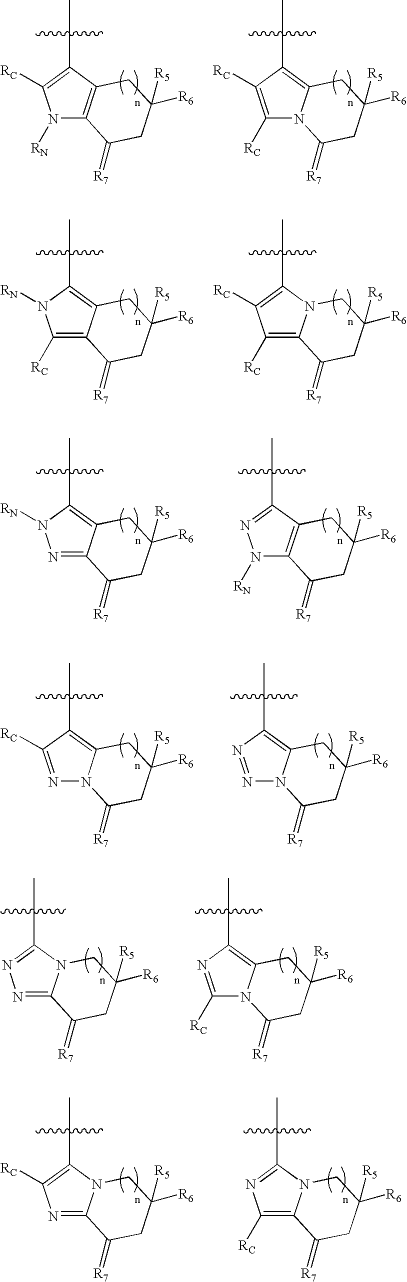

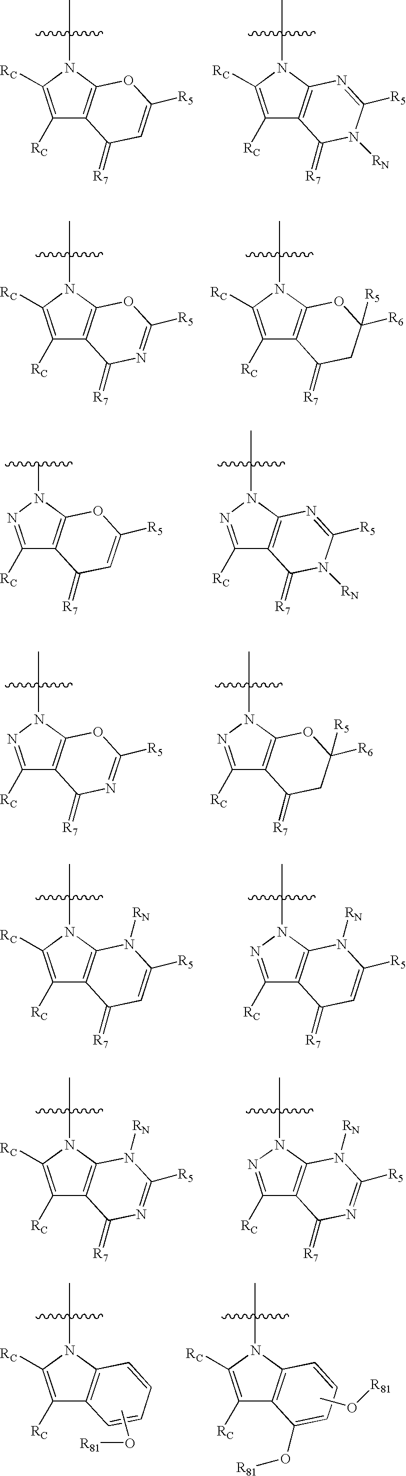

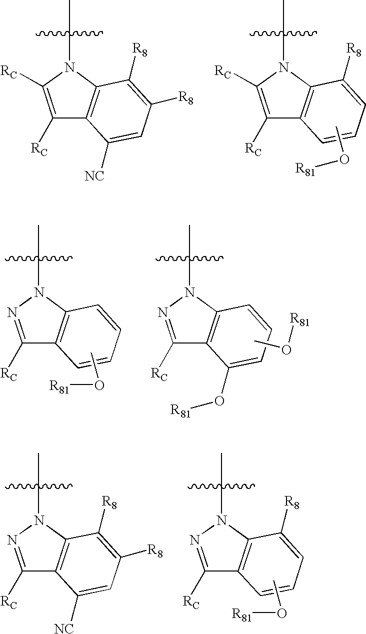

In one embodiment, the invention provides compounds according to formula (I) wherein A is one of the following structures,

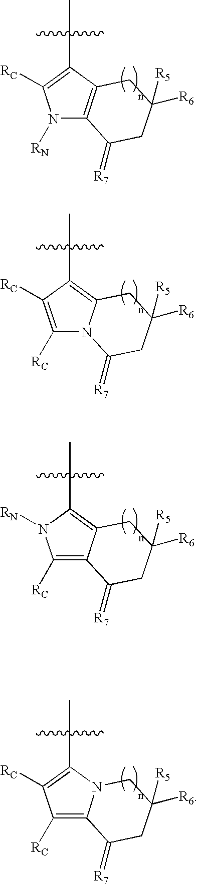

In one embodiment, the invention provides compounds according to formula (I) wherein A is one of the following structures,

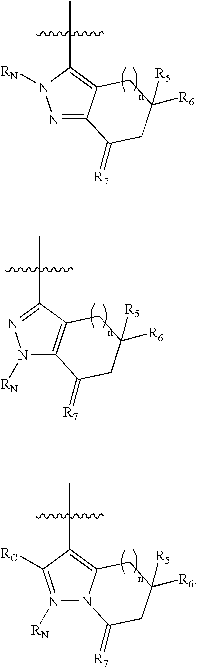

In one embodiment, the invention provides compounds according to formula (I) wherein A is one of the following structures,

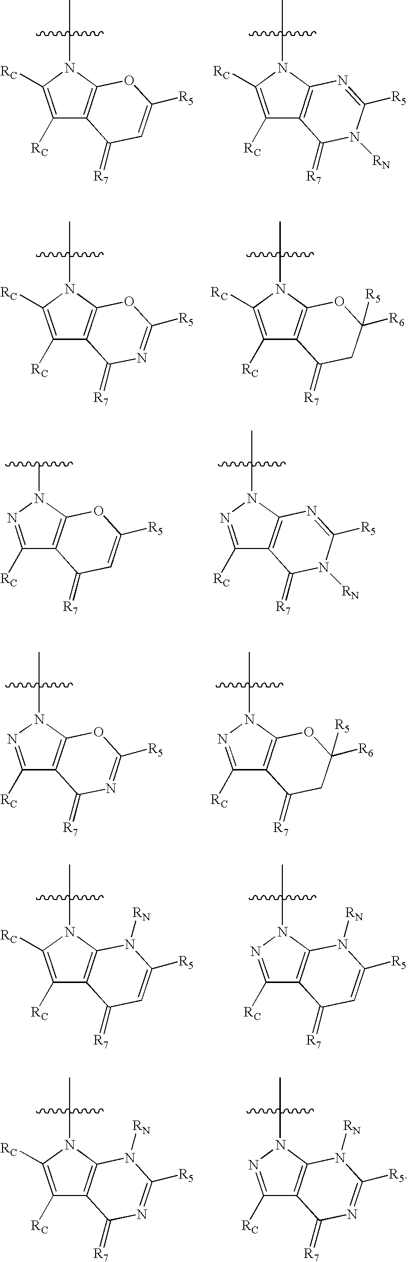

In one embodiment, the invention provides compounds according to formula (I) wherein A is one of the following structures,

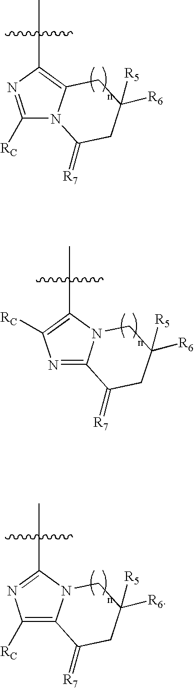

In one embodiment, the invention provides compounds according to formula (I) wherein A is one of the following structures,

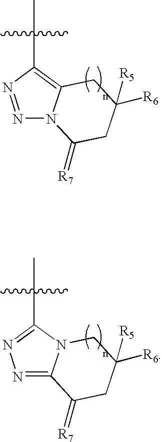

In one embodiment, the invention provides compounds according to formula (I) wherein A is one of the following structures, added 6 more here; added to the grand table

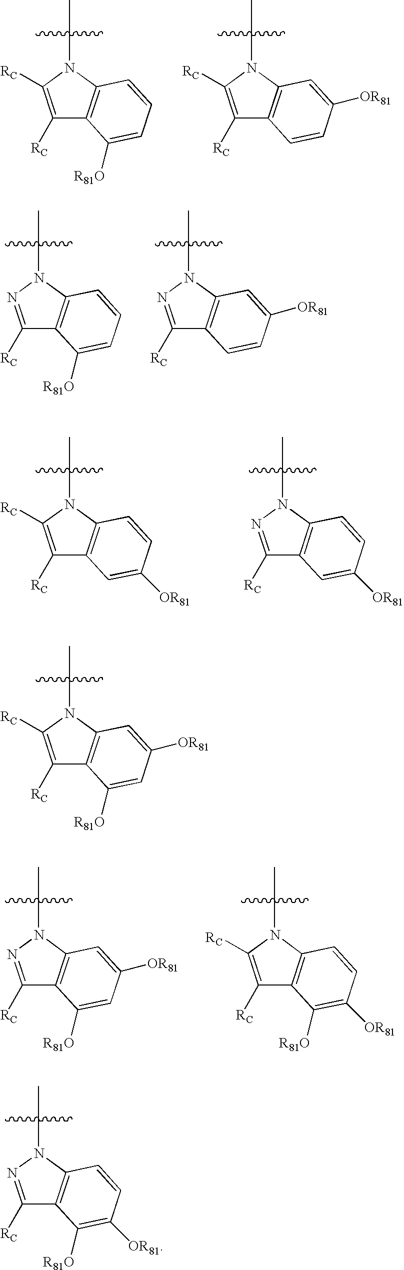

Preferred compounds of Formula I include those where R₅ and R₆ are independently H, C₁-C₆ alkyl, or aryl.

More preferred compounds of Formula I include those where R₅ and R₆ are independently H or C₁-C₆ alkyl.

Preferred compounds of Formula I include those where R₇ is O or N—OH.

Other preferred compounds of Formula I include those where R₂₁ is cyano.

More preferred compounds of Formula I include those where R₂₁ is —C(O)N(R₁₁₁)₂, wherein
each R₁₁₁ is independently H, hydroxy, C₁-C₆ alkyl, aryl, or C₃-C₈ cycloalkyl,
   wherein each R₁₁₁ is optionally substituted with from 1 to 4 R groups.

Even more preferred compounds of Formula I include those where R₂₁ is —C(O)NH₂.

In another embodiment, the invention provides compounds of formula (II),

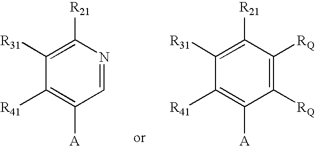

wherein R_{Q}, R₂₁, R₃₁, R₄₁, and A are as defined for formula (I).

In a preferred embodiment, the invention provides compounds according to formula II, wherein each R_{Q} is independently hydrogen, —F, —Cl, methoxy or ethoxy.

In a more preferred embodiment, the invention provides compounds according to formula II, wherein each R_{Q} is independently hydrogen, or —F.

In another embodiment, the invention provides compounds according to formula II, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
   wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂.

In another embodiment, the invention provides compounds according to formula II, wherein
R₅ and R₆ are independently H, C₁-C₆ alkyl, or aryl,

wherein the aryl is optionally substituted with from 1 to 4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide.

In another embodiment, the invention provides compounds according to formula II, wherein
R₇ is O or N—OH.

Other preferred compounds of Formula II include those where R₂₁ is cyano.

More preferred compounds of Formula II include those where R₂₁ is —C(O)N(R₁₁₁)₂, wherein
each R₁₁₁ is independently H, hydroxy, C₁-C₆ alkyl, aryl, or C₃-C₈ cycloalkyl,
   wherein each R₁₁₁ is optionally substituted with from 1 to 4 R groups.

Even more preferred compounds of Formula II include those where R₂₁ is —C(O)NH₂.

In another embodiment, the invention provides compounds according to formula II, wherein
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

In one embodiment, the invention provides compounds according to formula (II) wherein A is

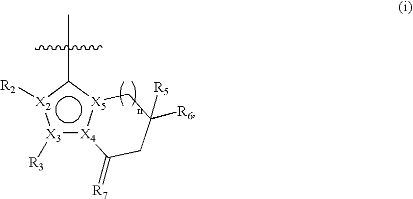

such compounds are designated hereafter as formula (II-a).

In a preferred embodiment, the invention provides compounds according to formula (II) wherein A is

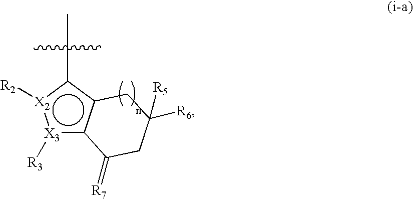

such compounds are designated hereafter as formula (II-b).

In another preferred embodiment, the invention provides compounds according to formula (II) wherein A is

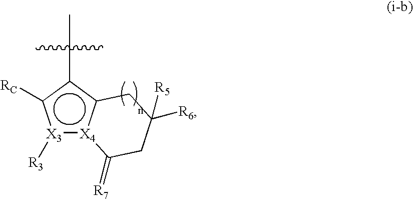

such compounds are designated hereafter as formula (II-c).

In another preferred embodiment, the invention provides compounds according to formula (II) wherein A is

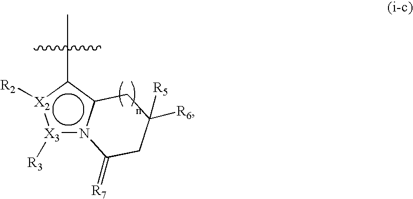

such compounds are designated hereafter as formula (II-d).

In another preferred embodiment, the invention provides compounds according to formula (II) wherein A is

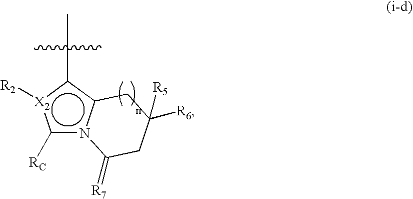

such compounds are designated hereafter as formula (II-e).

In another preferred embodiment, the invention provides compounds according to formula (II) wherein A is

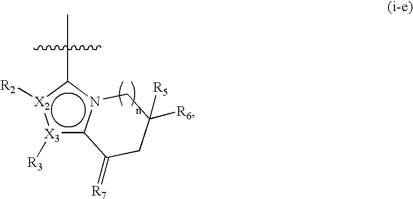

such compounds are designated hereafter as formula (II-f).

In another preferred embodiment, the invention provides compounds according to formula (II) wherein A is

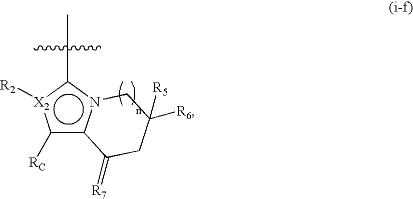

such compounds are designated hereafter as formula (II-g).

In one embodiment, the invention provides compounds according to formula (II) wherein A is

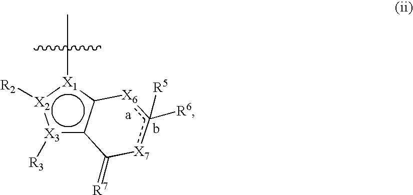

such compounds are designated hereafter as formula (II-h).

In a preferred embodiment, the invention provides compounds according to formula (II) wherein A is

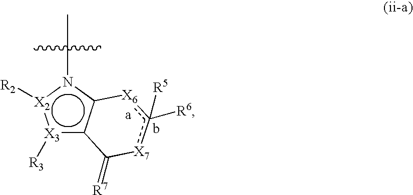

such compounds are designated hereafter as formula (II-i).

In more preferred embodiment, the invention provides compounds according to formula (II) wherein A is

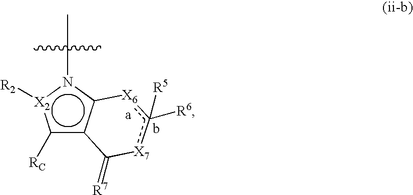

such compounds are designated hereafter as formula (II-j).

In another more preferred embodiment, the invention provides compounds according to formula (II) wherein A is

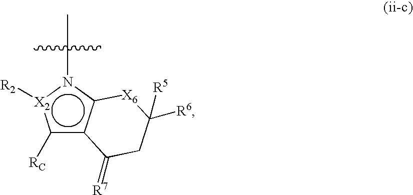

such compounds are designated hereafter as formula (II-k).

In another more preferred embodiment, the invention provides compounds according to formula (II) wherein A is

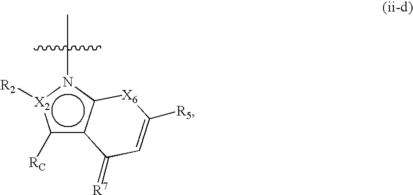

such compounds are designated hereafter as formula (II-1).

In another more preferred embodiment, the invention provides compounds according to formula (II) wherein A is

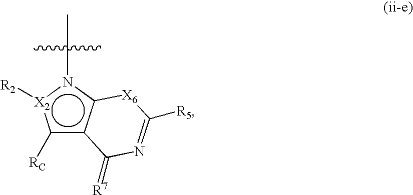

such compounds are designated hereafter as formula (II-m).

In one embodiment, the invention provides compounds according to formula (II) wherein A is

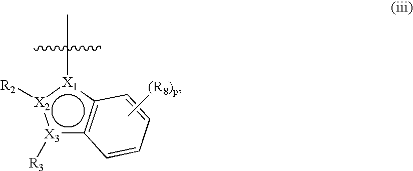

such compounds are designated hereafter as formula (II-n).

In a preferred embodiment, the invention provides compounds according to formula (II) wherein A is

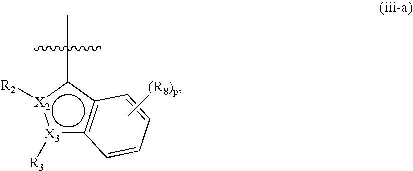

such compounds are designated hereafter as formula (II-o).

In a another preferred embodiment, the invention provides compounds according to formula (II) wherein A is

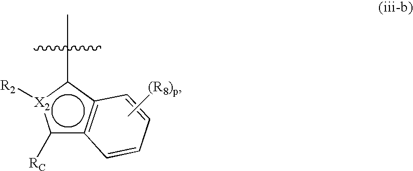

such compounds are designated hereafter as formula (II-p).

In a another preferred embodiment, the invention provides compounds according to formula (II) wherein A is

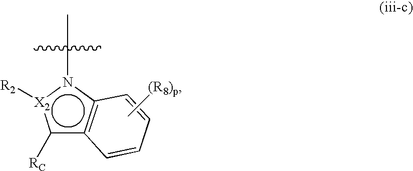

such compounds are designated hereafter as formula (II-q).

In one embodiment, the invention provides compounds according to formula (II) wherein A is

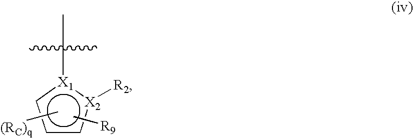

such compounds are designated hereafter as formula (II-r).

In one embodiment, the invention provides compounds according to formula (II) wherein A is

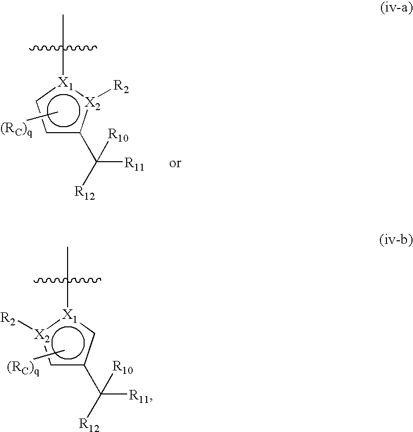

such compounds are designated hereafter as formula (II-s).

In a preferred embodiment, the invention provides compounds according to formula (II) wherein A is

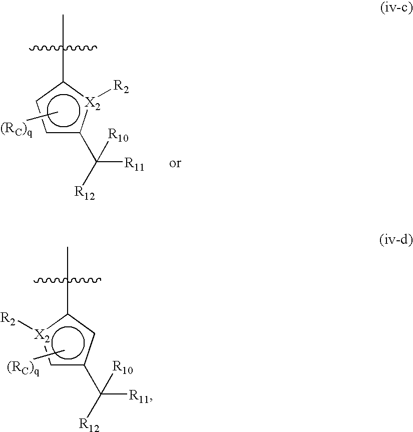

such compounds are designated hereafter as formula (II-t).

In a another preferred embodiment, the invention provides compounds according to formula (II) wherein A is

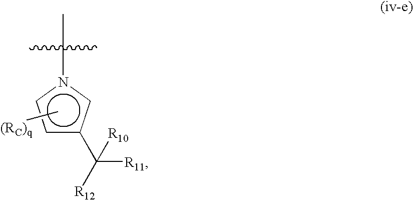

such compounds are designated hereafter as formula (II-u).

In one embodiment, the invention provides compounds according to formula (II) wherein A is

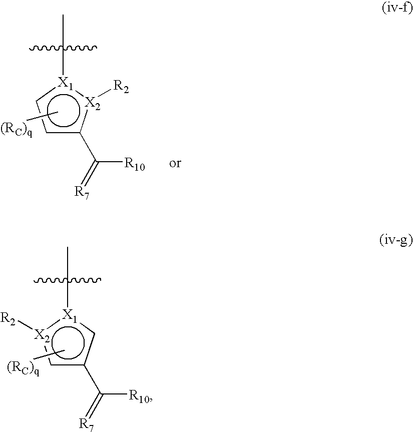

such compounds are designated hereafter as formula (II-v).

Preferred compounds of formula (II) include those wherein A is one of the following,

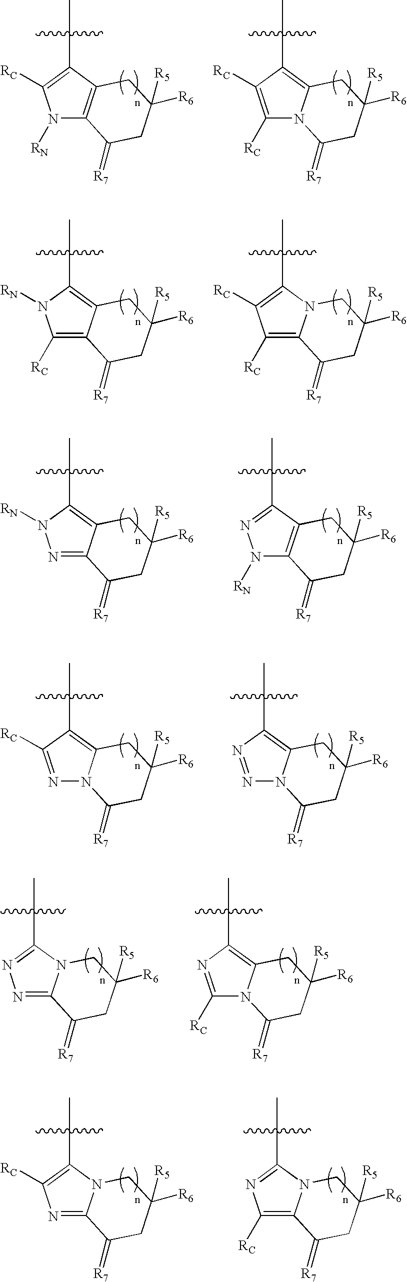

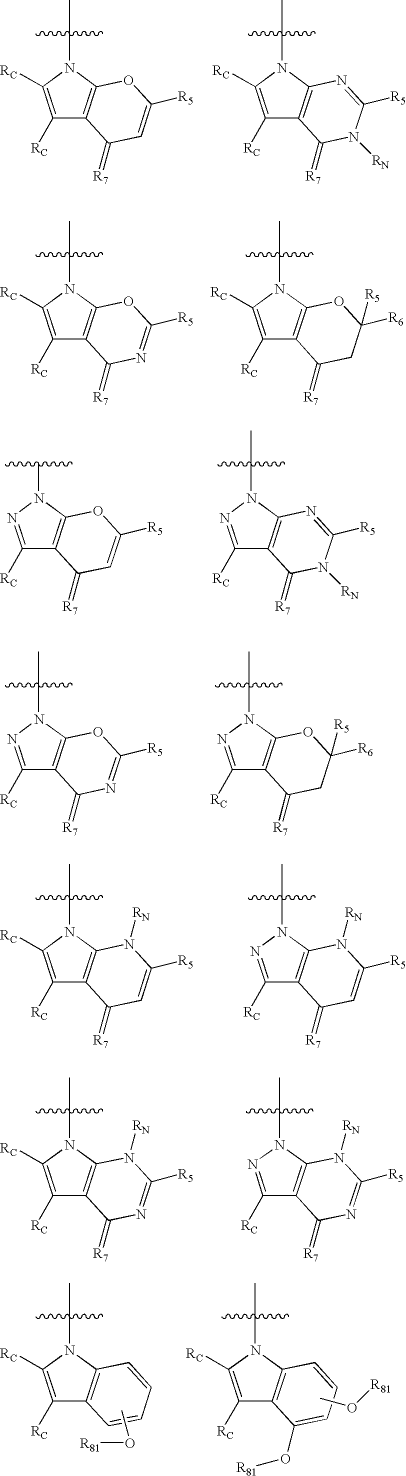

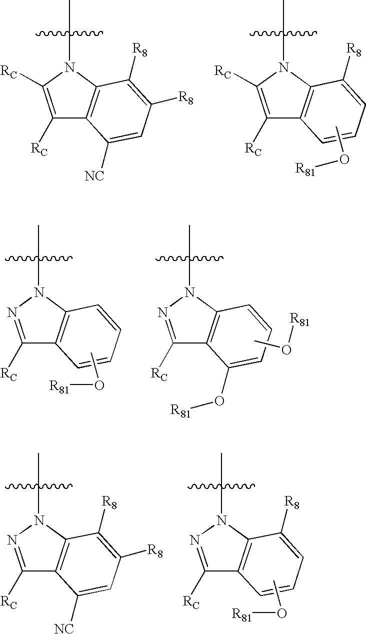

such compounds are designated hereafter as formula (II-w).

More preferred compounds of formula (II) include those wherein A is one of the following,

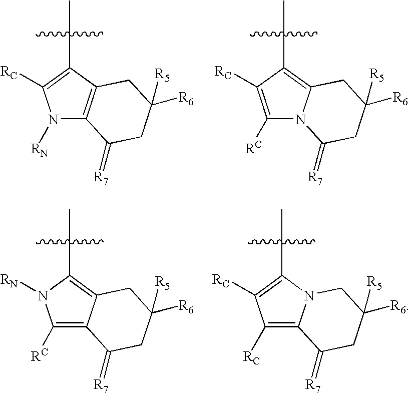

such compounds are designated hereafter as formula (II-x).

Other more preferred compounds of formula (II) include those wherein A is one of the following,

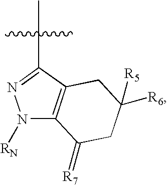

such compounds are designated hereafter as formula (II-y).

Other more preferred compounds of formula (II) include those wherein A is one of the following,

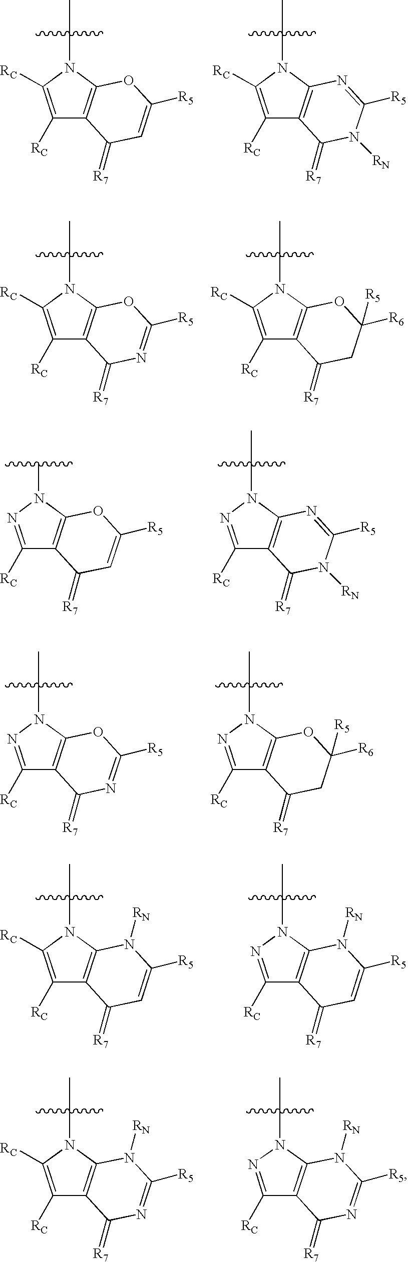

such compounds are designated hereafter as formula (II-z).

Other more preferred compounds of formula (II) include those wherein A is one of the following,

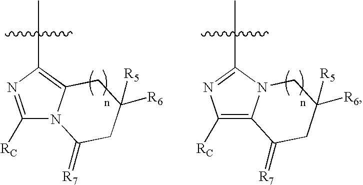

such compounds are designated hereafter as formula (II-aa).

Other more preferred compounds of formula (II) include those wherein A is one of the following,

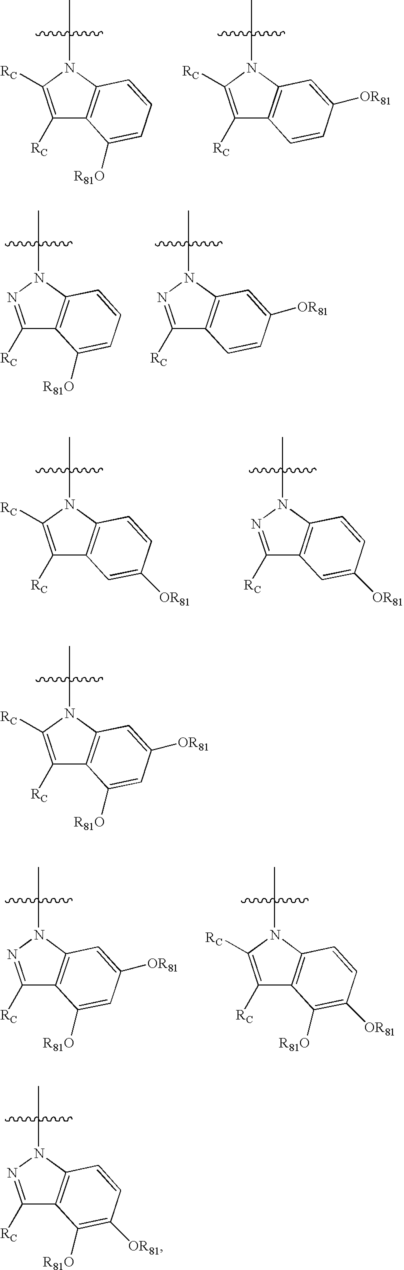

such compounds are designated hereafter as formula (II-ab).

Other more preferred compounds of formula (II) include those wherein A is one of the following,

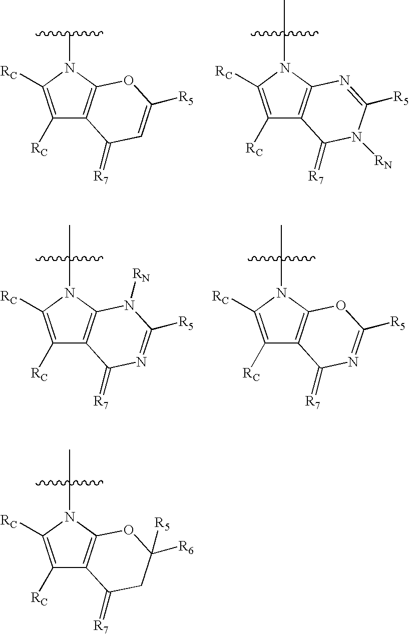

such compounds are designated hereafter as formula (II-ac).

Other more preferred compounds of formula (II) include those wherein A is one of the following,

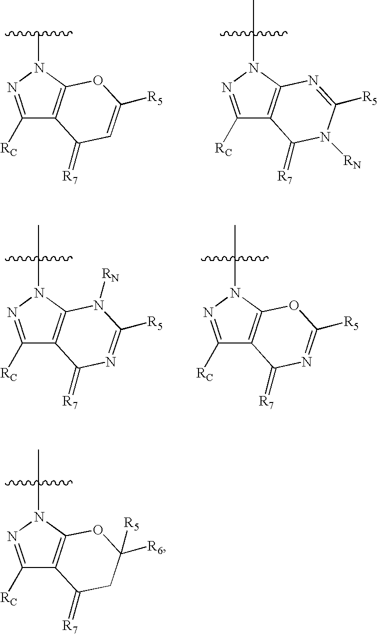

such compounds are designated hereafter as formula (II-ad).

In another embodiment, the invention provides compounds according to any of formulas IIa-IIz, and II-aa-II-ad, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
   wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂.

In another embodiment, the invention provides compounds according to any of formulas IIa-IIz, and II-aa-II-ad, wherein
R₅ and R₆ are independently H, C₁-C₆ alkyl, or aryl, wherein the aryl is optionally substituted with from 1 to 4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide.

In another embodiment, the invention provides compounds according to any of formulas IIa-IIz, and II-aa-II-ad, wherein

R₇ is O or N—OH.

In another embodiment, the invention provides compounds according to any of formulas IIa-IIz, and II-aa-II-ad, wherein

R₂₁ is —C(O)N(R₁₁₁)₂, wherein
each R₁₁₁ is independently H, hydroxy, C₁-C₆ alkyl, aryl, or C₃-C₈ cycloalkyl,
   wherein each R₁₁₁ is optionally substituted with from 1 to 4 R groups.

In another embodiment, the invention provides compounds according to any of formulas IIa-IIz, and II-aa-II-ad, wherein
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

Preferred compounds of formulas IIa-IIz, and II-aa-II-ad, include those where R_{N} is —H, C₁-C₆ alkyl or halo(C₁-C₆)alkyl.

In another embodiment, the invention provides compounds according to any of formulas IIa-IIz, and II-aa-II-ad, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
   wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂;
R₅ and R₆ are independently H, C₁-C₆ alkyl, or aryl,

wherein the aryl is optionally substituted with from 1 to 4 groups that are independently C₁-C₆ alkyl, C₁-C₆ alkoxy, halogen, hydroxy, amino, mono- or di-(C₁-C₆)alkylamino, nitro, halo(C₁-C₆)alkyl, halo(C₁-C₆)alkoxy, or carboxamide;

### R₇ is O or N—OH;

### R₂₁ is —C(O)N(R₁₁₁)₂, wherein

each R₁₁₁ is independently H, hydroxy, C₁-C₆ alkyl, aryl, or C₃-C₈ cycloalkyl,
   wherein each R₁₁₁ is optionally substituted with from 1 to 4 R groups; and
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

Preferred compounds of formulas IIa-IIz, and II-aa-II-ad include those that carry the definitions set forth in the previous paragraph for R₃₁, R₄₁, R₅, R₆, R₇, and R₂₁ and where R_{N} is —H, C₁-C₆ alkyl or halo(C₁-C₆)alkyl.

In another embodiment, the invention provides compounds according to formula II, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
   wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂;

### R₅ and R₆ are independently H, C₁-C₆ alkyl, or aryl;

### R₇ is O or N—OH;

### R₂₁ is cyano or —C(O)N(R₁₁₁)₂, wherein

each R₁₁₁ is independently H, hydroxy, C₁-C₆ alkyl, aryl, or C₃-C₈ cycloalkyl,
wherein each R₁₁₁ is optionally substituted with from 1 to 4 R groups; and
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl,
   such compounds are referred to hereafter as formula (III).

Preferred compounds of formula III include those where R_{N} is —H, C₁-C₆ alkyl or halo(C₁-C₆)alkyl.

In one embodiment, the invention provides compounds according to formula (III) wherein A is

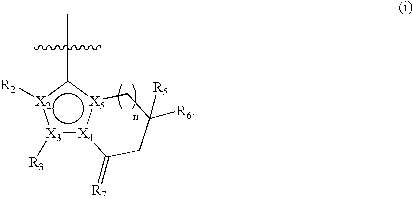

In a preferred embodiment, the invention provides compounds according to formula (III) wherein A is

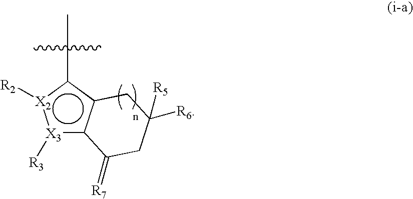

In another preferred embodiment, the invention provides compounds according to formula (III) wherein A is

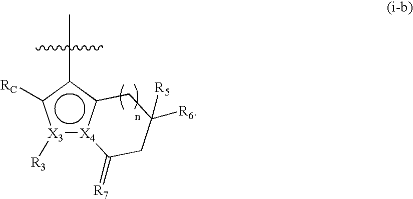

In another preferred embodiment, the invention provides compounds according to formula (III) wherein A is

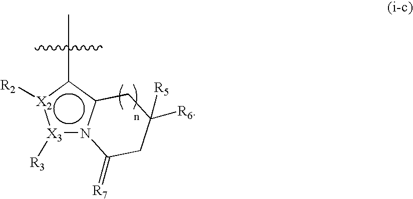

In another preferred embodiment, the invention provides compounds according to formula (III) wherein A is

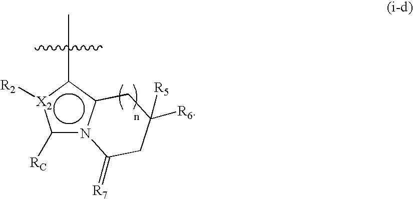

In another preferred embodiment, the invention provides compounds according to formula (III) wherein A is

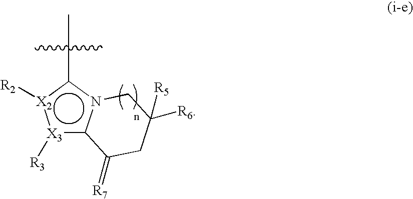

In another preferred embodiment, the invention provides compounds according to formula (III) wherein A is

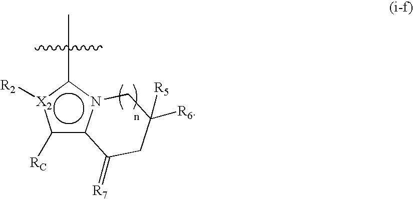

In one embodiment, the invention provides compounds according to formula (II) wherein A is

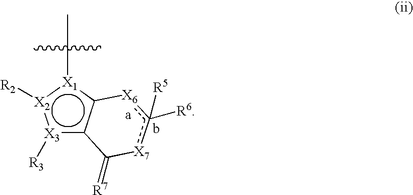

In a preferred embodiment, the invention provides compounds according to formula (III) wherein A is

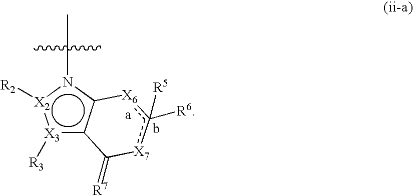

In more preferred embodiment, the invention provides compounds according to formula (III) wherein A is

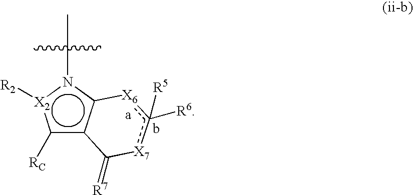

In another more preferred embodiment, the invention provides compounds according to formula (III) wherein A is

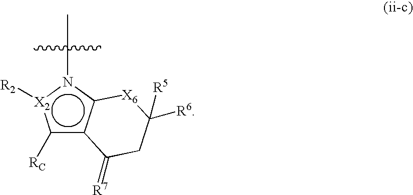

In another more preferred embodiment, the invention provides compounds according to formula (III) wherein A is

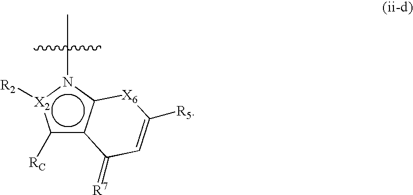

In another more preferred embodiment, the invention provides compounds according to formula (II) wherein A is

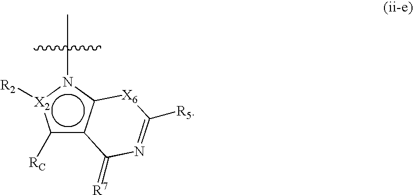

In one embodiment, the invention provides compounds according to formula (III) wherein A is

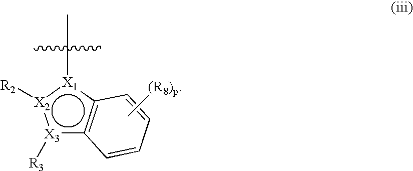

In a preferred embodiment, the invention provides compounds according to formula (III) wherein A is

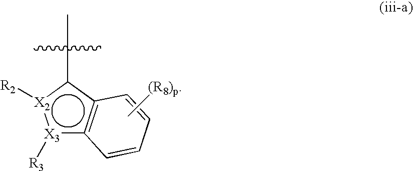

In a another preferred embodiment, the invention provides compounds according to formula (III) wherein A is

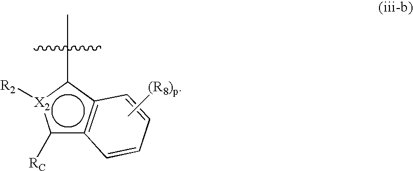

In a another preferred embodiment, the invention provides compounds according to formula (III) wherein A is

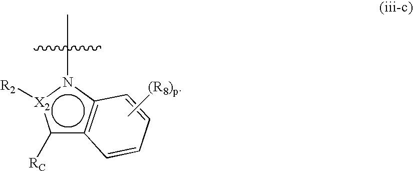

In one embodiment, the invention provides compounds according to formula (III) wherein A is

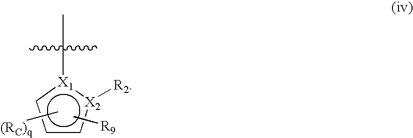

In a preferred embodiment, the invention provides compounds according to formula (III) wherein A is

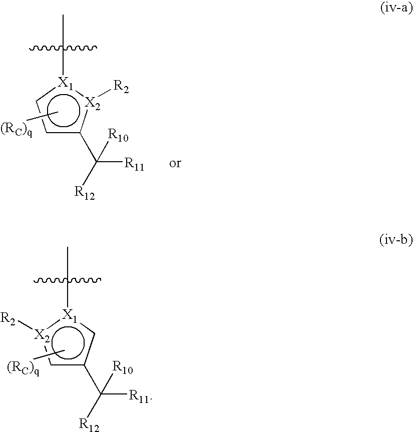

In a preferred embodiment, the invention provides compounds according to formula (III) wherein A is

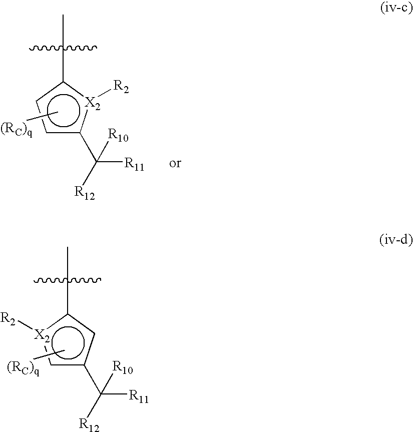

In a another preferred embodiment, the invention provides compounds according to formula (III) wherein A is

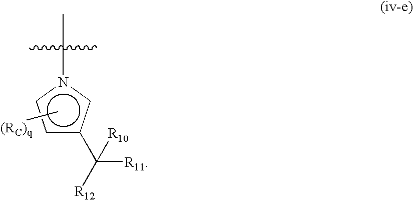

In one embodiment, the invention provides compounds according to formula (III) wherein A is

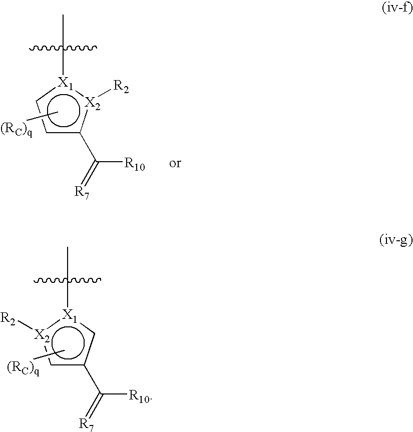

In a preferred embodiment, the invention provides compounds according to formula (III), wherein

### A is

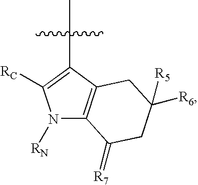

such compounds are referred to hereafter as formula (III-a).

In a preferred embodiment, the invention provides compounds according to formula (III), wherein

### A is

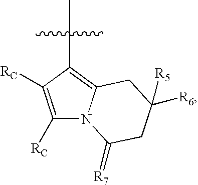

such compounds are referred to hereafter as formula (III-b).

In a preferred embodiment, the invention provides compounds according to formula (III), wherein

### A is

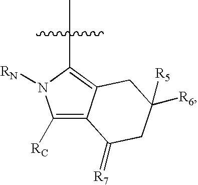

such compounds are referred to hereafter as formula (III-c).

In a preferred embodiment, the invention provides compounds according to formula (III), wherein

### A is

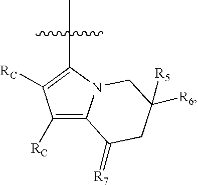

such compounds are referred to hereafter as formula (III-d).

In a preferred embodiment, the invention provides compounds according to formula (III), wherein

### A is

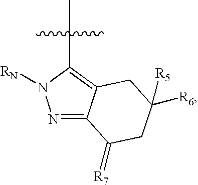

such compounds are referred to hereafter as formula (III-e).

In a preferred embodiment, the invention provides compounds according to formula (III), wherein

### A is

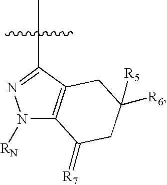

such compounds are referred to hereafter as formula (III-f).

In a preferred embodiment, the invention provides compounds according to formula (III), wherein

### A is

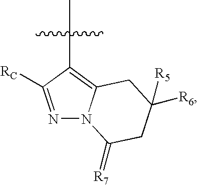

such compounds are referred to hereafter as formula (III-g).

In a preferred embodiment, the invention provides compounds according to formula (III), wherein

### A is

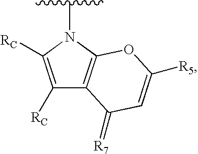

such compounds are referred to hereafter as formula (III-h).

In a preferred embodiment, the invention provides compounds according to formula (III), wherein

### A is

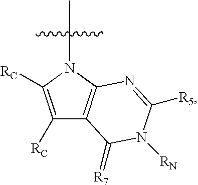

such compounds are referred to hereafter as formula (III-i).

In a preferred embodiment, the invention provides compounds according to formula (III), wherein

### A is

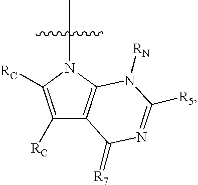

such compounds are referred to hereafter as formula (III-j).

In a preferred embodiment, the invention provides compounds according to formula (III), wherein

### A is

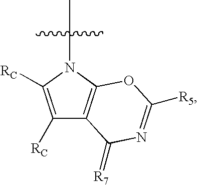

such compounds are referred to hereafter as formula (III-k).

In a preferred embodiment, the invention provides compounds according to formula (III), wherein

### A is

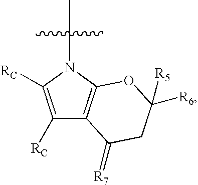

such compounds are referred to hereafter as formula (III-1).

In a preferred embodiment, the invention provides compounds according to formula (III), wherein

### A is

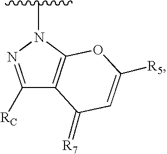

such compounds are referred to hereafter as formula (III-m).

In a preferred embodiment, the invention provides compounds according to formula (III), wherein

### A is

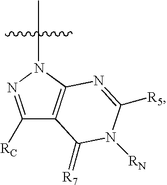

such compounds are referred to hereafter as formula (III-n).

In a preferred embodiment, the invention provides compounds according to formula (III), wherein

### A is

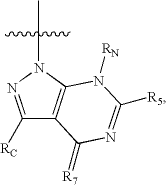

such compounds are referred to hereafter as formula (III-o).

In a preferred embodiment, the invention provides compounds according to formula (III), wherein

### A is

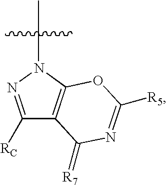

such compounds are referred to hereafter as formula (III-p).

In a preferred embodiment, the invention provides compounds according to formula (III), wherein

### A is

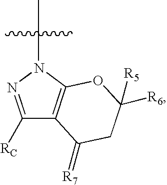

such compounds are referred to hereafter as formula (III-q).

In a preferred embodiment, the invention provides compounds according to formula (III), wherein

### A is

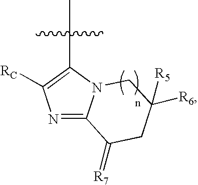

such compounds are referred to hereafter as formula (III-r).

In a preferred embodiment, the invention provides compounds according to formula (III), wherein

A is

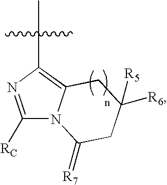

such compounds are referred to hereafter as formula (III-s).

In a preferred embodiment, the invention provides compounds according to formula (III), wherein

### A is

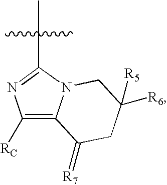

such compounds are referred to hereafter as formula (III-t).

In a preferred embodiment, the invention provides compounds according to formula (III), wherein

### A is

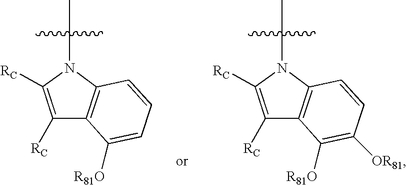

such compounds are referred to hereafter as formula (III-u).

In a preferred embodiment, the invention provides compounds according to formula (III), wherein

### A is

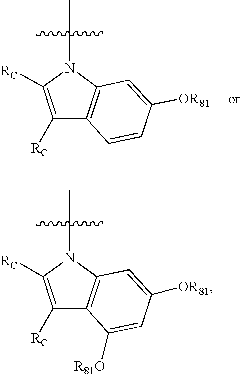

such compounds are referred to hereafter as formula (III-v).

In a preferred embodiment, the invention provides compounds according to formula (III), wherein

### A is

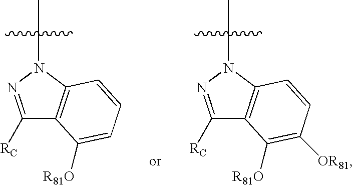

such compounds are referred to hereafter as formula (III-w).

In a preferred embodiment, the invention provides compounds according to formula (III), wherein

### A is

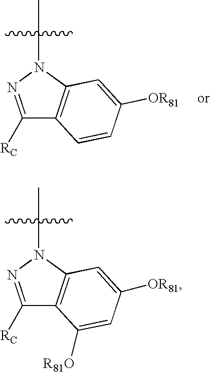

such compounds are referred to hereafter as formula (III-x).

In a preferred embodiment, the invention provides compounds according to formula (III), wherein

### A is

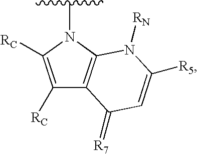

such compounds are referred to hereafter as formula (III-y).

In a preferred embodiment, the invention provides compounds according to formula (III), wherein

### A is

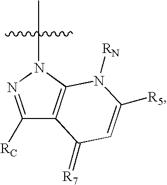

such compounds are referred to hereafter as formula (III-z).

In a preferred embodiment, the invention provides compounds according to formula (III), wherein

### A is

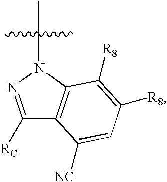

such compounds are referred to hereafter as formula (III-aa).

In a preferred embodiment, the invention provides compounds according to formula (III), wherein

### A is

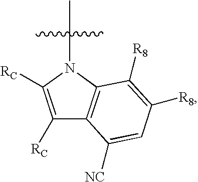

such compounds are referred to hereafter as formula (III-ab).

In a preferred embodiment, the invention provides compounds according to formula (III), wherein

### A is

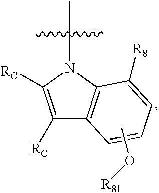

wherein R₈ is halogen or C₁-C₆ alkyl; such compounds are referred to hereafter as formula (III-ac).

In a preferred embodiment, the invention provides compounds according to formula (III), wherein

### A is

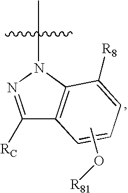

wherein R₈ is halogen or C₁-C₆ alkyl; such compounds are referred to hereafter as formula (III-ad).

In a preferred embodiment, the invention provides compounds according to any of formulas (III-a-III-t) and (III-y-III-z), wherein R₇ is O.

In a preferred embodiment, the invention provides compounds according to any of formulas (III-a-III-t) and (III-y-III-z), wherein R₇ is O; and

each R_{C} is independently —H, —CH₂CH₃, —CH₃, —CFH₂, —CF₂H, —CF₃, —CF₂CF₃, —CH₂CF₃, cyclopropyl, or cyclopropylmethyl.

In a preferred embodiment, the invention provides compounds according to any of formulas (III-a-III-t) and (III-y-III-z), wherein R₇ is O; and

each R_{N} is independently —H, —CH₂CH₃, —CH₃, —CFH₂, —CF₂H, —CF₃, —CF₂CF₃, —CH₂CF₃, cyclopropyl, or cyclopropylmethyl.

In a preferred embodiment, the invention provides compounds according to any of formulas (III-a-III-t) and (III-y-III-z), wherein R₇ is O; and

each R_{C} and R_{N} independently is —H, —CH₂CH₃, —CH₃, —CFH₂, —CF₂H, —CF₃, —CF₂CF₃, —CH₂CF₃, cyclopropyl, or cyclopropylmethyl.

In a preferred embodiment, the invention provides compounds according to any of formulas (III-a-III-z) and (III-aa-IIIad), wherein each R_{C} is independently —H, —CH₂CH₃, —CH₃, —CFH₂, —CF₂H, —CF₃, —CF₂CF₃, —CH₂CF₃, cyclopropyl, or cyclopropylmethyl.

In a preferred embodiment, the invention provides compounds according to any of formulas (III-a-III-z) and (III-aa-IIIad), wherein each R_{N} is independently —H, —CH₂CH₃, —CH₃, —CFH₂, —CF₂H, —CF₃, —CF₂CF₃, —CH₂CF₃, cyclopropyl, or cyclopropylmethyl.

In a preferred embodiment, the invention provides compounds according to any of formulas (III-a-III-z) and (III-aa-IIIad), wherein each R_{C} and R_{N} independently is —H, —CH₂CH₃, —CH₃, —CFH₂, —CF₂H, —CF₃, —CF₂CF₃, —CH₂CF₃, cyclopropyl, or cyclopropylmethyl.

Preferred compounds of Formulas III-a-III-Z include those where R₅ and R₆ are independently H, C₁-C₆ alkyl, or aryl.

Particular compounds of Formulas III-e, III-f, and III-g include those where R₅ and R₆ are independently H, C₁-C₆ alkyl, or aryl. Other particular compounds of Formulas III-e, III-f, and III-g include those where R₅ and R₆ are independently H, C₁-C₆ alkyl, or aryl; and R₇ is O, Still other particular compounds of Formulas III-e, III-f, and III-g include those where R₅ and R₆ are independently H, C₁-C₆ alkyl, or aryl; R₇ is O; and R_{C} and R_{N} are independently —H, —CH₂CH₃, —CH₃, —CFH₂, —CF₂H, —CF₃, —CF₂CF₃, —CH₂CF₃, cyclopropyl, or cyclopropylmethyl.

In a preferred embodiment, the invention provides compounds according to any of formulas (III-a-III-z) and (III-aa-IIIad), wherein R₂₁ is cyano.

Particular compounds of formulas III-e, III-f, and III-g include those where R₅ and R₆ are independently H, C₁-C₆ alkyl, or aryl; R₇ is O; and R₂₁ is cyano or —C(O)NH₂. Still other particular compounds of Formulas III-e, III-f, and III-g include those where R₅ and R₆ are independently H, C₁-C₆ alkyl, or aryl; R₇ is O; R_{C} and R_{N} are independently —H, —CH₂CH₃, —CH₃, —CFH₂, —CF₂H, —CF₃, —CF₂CF₃, —CH₂CF₃, cyclopropyl, or cyclopropylmethyl; and R₂₁ is cyano or —C(O)NH₂.

In a preferred embodiment, the invention provides compounds according to any of formulas (III-a-III-z and (III-aa-IIIad)), wherein R₂₁ is —C(O)NH₂.

Other preferred compounds of formula (II) include those wherein A is one of the following,

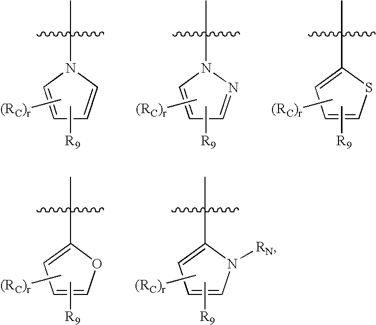

wherein r is 0 or 1; such compounds are designated hereafter as formula (IV).

In another embodiment, the invention provides compounds according to formula IV, wherein R₉ is C₁-C₆ alkoxy.

In another embodiment, the invention provides compounds according to formula IV, wherein

R₉ is —CH(OH)—R₁₀,

wherein
R₁₀ is C₁-C₆ alkyl optionally substituted with 1 or 2
R₁₀₁ groups, wherein
   each R₁₀₁ is independently halogen, nitro, cyano, C₁-C₆ alkyl, halo(C₁-C₆)alkyl, —OR_{O}, —N(R_{N})₂, —S(O)ₘR_{N′}, or —C(O)R_{N′}.

In another embodiment, the invention provides compounds according to formula IV, wherein

R₉ is —C(O)R₁₀,

wherein
R₁₀ is C₁-C₆ alkyl or halo(C₁-C₆)alkyl, either optionally substituted with 1 or 2 R₁₀₁ groups, wherein
each R₁₀₁ is independently halogen, nitro, cyano, C₁-C₆ alkyl, halo(C₁-C₆)alkyl, —OR_{O}, —N(R_{N})₂, —S(O)ₘR_{N′}, or —C(O)R_{N′}.

In another embodiment, the invention provides compounds according to formula IV, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
   wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂.

In another embodiment, the invention provides compounds according to formula IV, wherein R₂₁ is cyano.

In another embodiment, the invention provides compounds according to formula IV, wherein

R₂₁ is —C(O)N(R₁₁₁)₂, wherein
each R₁₁₁ is independently H, hydroxy, C₁-C₆ alkyl, aryl, or C₃-C₈ cycloalkyl,
   wherein each R₁₁₁ is optionally substituted with from 1 to 4 R groups.

In a preferred embodiment, the invention provides compounds according to formula IV, wherein R₂₁ is —C(O)NH₂.

In another embodiment, the invention provides compounds according to formula IV, wherein
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

In another embodiment, the invention provides compounds according to formula IV, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
   wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂;

### R₂₁ is cyano or —C(O)NH₂; and

each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

More preferred compounds of formula (IV) include those wherein A is

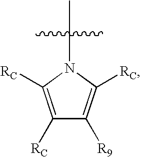

such compounds are designated hereafter as formula (V-a).

More preferred compounds of formula (IV) include those wherein A is

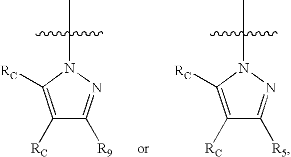

such compounds are designated hereafter as formula (V-b).

More preferred compounds of formula (IV) include those wherein A is

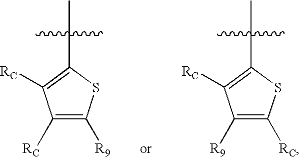

such compounds are designated hereafter as formula (V-c).

More preferred compounds of formula (IV) include those wherein A is

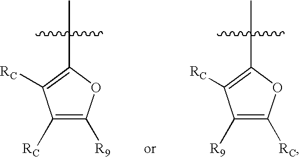

such compounds are designated hereafter as formula (V-d).

More preferred compounds of formula (IV) include those wherein A is

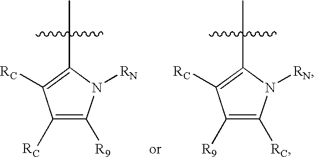

such compounds are designated hereafter as formula (V-e).

In more preferred embodiments, the invention provides compounds according to any of formulas V-a-V-e, wherein R_{N} and each R_{C} is independently —H, —CH₂CH₃, —CH₃, —CFH₂, —CF₂H, —CF₃, —CF₂CF₃, —CH₂CF₃, cyclopropyl, or cyclopropylmethyl.

In another embodiment, the invention provides compounds according to any of formulas V-a-V-e, wherein R₉ is C₁-C₆ alkoxy.

In another embodiment, the invention provides compounds according to any of formulas V-a-V-e, wherein

R₉ is —CH(OH)—R₁₀,

wherein
R₁₀ is C₁-C₆ alkyl optionally substituted with 1 or 2 R₁₀₁ groups, wherein
each R₁₀₁ is independently halogen, nitro, cyano, C₁-C₆ alkyl, halo(C₁-C₆)alkyl, —OR_{O}, —N(R_{N})₂, —S(O)ₘR_{N′}, or —C(O)R_{N′}.

In another embodiment, the invention provides compounds according to any of formulas V-a-V-e, wherein

R₉ is —C(O)R₁₀,

wherein
R₁₀ is C₁-C₆ alkyl or halo(C₁-C₆)alkyl, either optionally substituted with 1 or 2 R₁₀₁ groups, wherein
each R₁₀₁ is independently halogen, nitro, cyano, C₁-C₆ alkyl, halo(C₁-C₆)alkyl, —OR_{O}, —N(R_{N})₂, —S(O)ₘR_{N′}, or —C(O)R_{N′}.

In another embodiment, the invention provides compounds according to any of formulas V-a-V-e, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
   wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂.

In another embodiment, the invention provides compounds according to any of formulas V-a-V-e, wherein R₂₁ is cyano.

In another embodiment, the invention provides compounds according to any of formulas V-a-V-e, wherein

R₂₁ is —C(O)N(R₁₁₁)₂, wherein
each R₁₁₁ is independently H, hydroxy, C₁-C₆ alkyl, aryl, or C₃-C₈ cycloalkyl,
   wherein each R₁₁₁ is optionally substituted with from 1 to 4 R groups.

In a preferred embodiment, the invention provides compounds according to any of formulas V-a-V-e, wherein R₂₁ is —C(O)NH₂.

In another embodiment, the invention provides compounds according to any of formulas V-a-V-e, wherein
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

In another embodiment, the invention provides compounds according to any of formulas V-a-V-e, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
   wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂;

### R₂₁ is cyano or —C(O)NH₂; and

each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

The invention further encompasses intermediates useful for preparing compounds of Formula I. These include compounds of formulas VI-XIII, presented below.

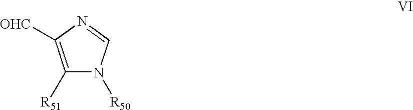

where
R₅₀ is a nitrogen protecting group; and

R₅₁ is C₁-C₁₀ alkyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl.

Preferred compounds of formula VI are those wherein R₅₀ is triphenylmethyl.

Other preferred compounds of formula VI are those wherein

R₅₁ is C₁-C₆ alkyl, C₁-C₆ haloalkyl, C₃-C₇ cycloalkyl, or C₃-C₇ cycloalkyl(C₁-C₁₀) alkyl.

More preferred compounds of formula VI are those wherein R₅₀ is triphenylmethyl and R₅₁ is C₁-C₆ alkyl, C₁-C₆ haloalkyl, C₃-C₇ cycloalkyl, or C₃-C₇ cycloalkyl(C₁-C₁₀) alkyl.

Even more preferred compounds of formula VI are those wherein R₅₁ is C₁-C₃ alkyl, C₁-C₃ haloalkyl, C₃-C₅ cycloalkyl, or C₃-C₅ cycloalkyl(C₁-C₃)alkyl.

Other even more preferred compounds of formula VI are those wherein R₅₀ is triphenylmethyl and R₅₁ is C₁-C₃ alkyl, C₁-C₃ haloalkyl, C₃-C₅ cycloalkyl, or C₃-C₅ cycloalkyl(C₁-C₃)alkyl.

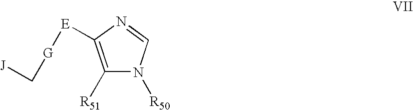

wherein
J is a protected hydroxy group;
E represents —CH(OH)— or —C(O)—;
G is C₂-alkynylene or C₂-alkylene;
R₅₀ is a nitrogen protecting group; and
R₅₁ is C₁-C₁₀ alkyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl.

Preferred compounds of formula VII are those wherein J is THP—O— and R₅₀ is triphenylmethyl.

More preferred compounds of formula VII are those wherein J is THP—O— and R₅₁ is C₁-C₆ alkyl, C₁-C₆ haloalkyl, C₃-C₇ cycloalkyl, or C₃-C₇ cycloalkyl(C₁-C₁₀)alkyl.

Other preferred compounds of formula VII are those wherein R₅₀ is triphenylmethyl, and R₅₁ is C₁-C₆ alkyl, C₁-C₆ haloalkyl, C₃-C₇ cycloalkyl, or C₃-C₇ cycloalkyl(C₁-C₁₀)alkyl.

Even more preferred compounds of formula VII are those wherein J is THP—O— and R₅₁ is C₁-C₃ alkyl, C₁-C₃ haloalkyl, C₃-C₅ cycloalkyl, or C₃-C₅ cycloalkyl(C₁-C₃)alkyl.

Yet other preferred compounds of formula VII are those wherein R₅₀ is triphenylmethyl, and R₅₁ is C₁-C₃ alkyl, C₁-C₃ haloalkyl, C₃-C₅ cycloalkyl, or C₃-C₅ cycloalkyl(C₁-C₃)alkyl.

Other preferred compounds of formula VII are those wherein R₅₀ is triphenylmethyl, R₅₁ is C₁-C₃ alkyl, C₁-C₃ haloalkyl, C₃-C₅ cycloalkyl, or C₃-C₅ cycloalkyl(C₁-C₃)alkyl, and E represents —CH(OH)—. Other more preferred compounds of formula VII are those wherein R₅₀ is triphenylmethyl, R₅₁ is C₁-C₃ alkyl, C₁-C₃ haloalkyl, C₃-C₅ cycloalkyl, or C₃-C₅ cycloalkyl(C₁-C₃)alkyl, and E represents —C(O)—.

Other preferred compounds of formula VII are those wherein G represents C₂-alkynylene. Other more preferred compounds of formula VII are those wherein G represents C₂-alkylene.

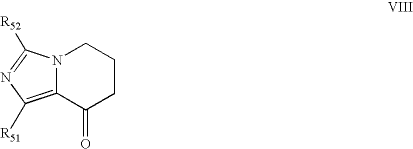

wherein
R₅₁ is C₁-C₁₀ alkyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl;
R₅₂ is
   halogen or
   phenyl that is
      substituted with one fluoro,
      optionally substituted with one of cyano, amino(C₁-C₆)alkyl, mono- or di(C₁-C₆)alkylamino(C₁-C₃)alkyl, —C(O)NR₅₃R₅₄ where R₅₃ and R₅₄ independently represent hydrogen or C₁-C₆ alkyl, and
      optionally substituted with one of C₁-C₆ alkyl, halo(C₁-C₆)alkyl, C₃-C₇cycloalkyl, bromo, iodo, chloro, nitro, or C₃-C₇ cycloalkyl(C₁-C₃)alkyl.

Preferred compounds of formula VIII are those wherein R₅₂ is hydrogen and R₅₁ is C₁-C₃ alkyl, C₁-C₃ haloalkyl, C₃-C₅ cycloalkyl, or C₃-C₅ cycloalkyl(C₁-C₃)alkyl.

More preferred compounds of formula VIII are those wherein R₅₂ is halogen and R₅₁ is C₁-C₃ alkyl, C₁-C₃ haloalkyl, C₃-C₅ cycloalkyl, or C₃-C₅ cycloalkyl(C₁-C₃)alkyl.

Even more preferred compounds of formula VIII are those wherein R₅₂ is bromo or chloro and R₅₁ is C₁-C₃ alkyl, C₁-C₃ haloalkyl, C₃-C₅ cycloalkyl, or C₃-C₅ cycloalkyl(C₁-C₃)alkyl.

Other preferred compounds of formula VIII are those wherein R₅₂ is phenyl substituted with fluoro and cyano, and R₅₁ is C₁-C₃ alkyl, C₁-C₃ haloalkyl, C₃-C₅ cycloalkyl, or C₃-C₅ cycloalkyl(C₁-C₃)alkyl.

Other even more preferred compounds of formula VIII are those wherein R₅₂ is

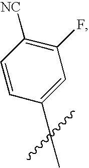

and

### R₅₁ is C₁-C₃ alkyl, C₁-C₃ haloalkyl, C₃-C₅ cycloalkyl, or C₃-C₅ cycloalkyl(C₁-C₃)alkyl.

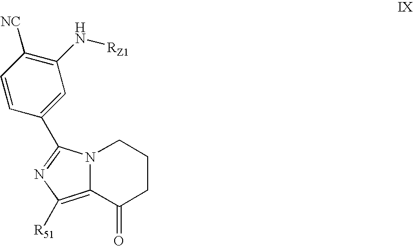

where
R₅₁ is C₁-C₁₀ alkyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl; and
   R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
   wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S(O)ₘ—(C₁-C₆)alkyl, —S(O)ₘ-aryl, —SO₂NH₂, —SO₂NH—(C₁-C₆)alkyl, —SO₂NH-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₂, where each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, where each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
   each R₂₂ is optionally fused to a C₆-C₁₀ aryl group, C₅-C₈ saturated cyclic group, or a C₅-C₁₀ heterocycloalkyl group.

Preferred compounds of formula IX are those wherein R₅₁ is C₁-C₃ alkyl, C₁-C₃ haloalkyl, C₃-C₅ cycloalkyl, or C₃-C₅ cycloalkyl(C₁-C₃)alkyl.

Other preferred compounds of formula IX are those wherein R_{Z1} is tetrahydro-pyran-4-yl, tetrahydro-furan-2-ylmethyl, 4-hydroxy-cyclohexyl, 1-methoxypropan-2-yl, phenyl, 2-methoxyethyl, or 2-hydroxycyclopentyl.

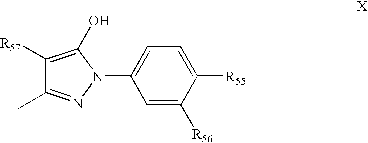

wherein
R₅₅ is cyano, amino(C₁-C₆)alkyl, mono- or di(C₁-C₆)alkylamino(C₁-C₃)alkyl, —C(O)NR₅₃R₅₄ where R₅₃ and R₅₄ independently represent hydrogen or C₁-C₆ alkyl;
R₅₆ is halogen; and
R₅₇ is hydrogen or 3-methylbut-2-enoyl.

Preferred compounds of formula X are those wherein R₅₅ is cyano or —C(O)NR₅₃R₅₄.

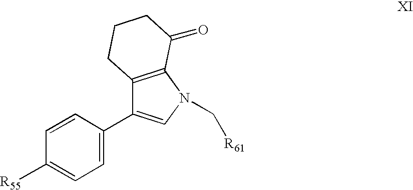

wherein
R₅₅ is cyano, amino(C₁-C₆)alkyl, mono- or di(C₁-C₆)alkylamino(C₁-C₃)alkyl, —C(O)NR₅₃R₅₄ where R₅₃ and R₅₄ independently represent hydrogen or C₁-C₆ alkyl; and
R₆₁ is phenyl substituted with 1-3 of hydroxy, C₁-C₆ alkoxy, C₁-C₆ alkyl, C₁-C₆ alkyl(C₁-C₆)alkoxy, or C₃-C₇ cycloalkyl(C₁-C₆)alkoxy.

Preferred compounds of formula XI are those wherein R₅₅ is cyano.

Other preferred compounds of formula XI are those wherein R₅₅ is cyano and R₆₁ is phenyl substituted with at least one of C₁-C₆ alkoxy.

More preferred compounds of formula XI are those wherein R₅₅ is cyano and R₆₁ is phenyl substituted with at least two of C₁-C₆ alkoxy.

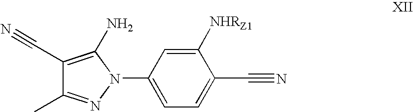

R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S(O)ₘ—(C₁-C₆)alkyl, —S(O)ₘ-aryl, —SO₂NH₂, —SO₂NH—(C₁-C₆)alkyl, —SO₂NH-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₂, where each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, where each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
each R₂₂ is optionally fused to a C₆-C₁₀ aryl group, C₅-C₈ saturated cyclic group, or a C₅-C₁₀ heterocycloalkyl group.

Preferred compounds of formula XII are those wherein R_{Z1} is tetrahydro-pyran-4-ylamino, tetrahydro-furan-2-ylmethylamino, 4-hydroxy-cyclohexylamino, phenylamino, or 2-hydroxycyclopentylamino.

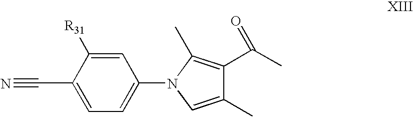

wherein
R₃₁ is (a) H, (b) halo, or (c) a C₁-C₁₅ alkyl group where up to six of the carbon atoms in said alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
   wherein each (c) is optionally substituted with —R_{C} —OR₁₅, —SR₁₅, —N(R₁₅)₂, or —R₂₂, wherein
      each R₁₅ is independently —H, (C₁-C₁₀)alkyl, (C₁-C₁₀) haloalkyl, (C₂-C₆)alkenyl, (C₂-C₆)alkynyl, or (C₁-C₁₀)alkyl-Z, wherein
         Z is —OR_{O} or —N(R₃₀)₂, wherein
            each R₃₀ is independently —H or C₁-C₆ alkyl;
            or N(R₃₀)₂ represents pyrrolidinyl, piperidinyl, piperazinyl, azepanyl, 1,3- or 1,4-diazepanyl, or morpholinyl, each of which is optionally substituted with R;
               each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆)alkylamino, carboxy, carboxamide, C₃-C₇cycloalkyl, heterocycloalkyl, aryl, or heteroaryl;
               each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, wherein
   each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
   each R₂₂ is optionally fused to a C₆-C₁₀ aryl group, C₅-C₈ saturated cyclic group, or a C₅-C₁₀ heterocycloalkyl group.

Preferred compounds of formula XIII are those wherein R₃₁ is amino substituted with
C₃-C₇ cycloalkyl substituted with amino, halogen, hydroxy, C₁-C₆ alkoxy, or nitro, or
a 4-7 membered heterocycloalkyl group optionally substituted with amino, halogen, hydroxy, C₁-C₆ alkoxy, or nitro.

More preferred compounds of formula XIII are those wherein R₃₁ is amino substituted with hydroxycyclohexyl or tetrahydropyranyl.

In another aspect, the invention encompasses a method of treating cancer comprising administering to a patient in need thereof, a pharmaceutically acceptable amount of a compound or salt of Formula I or a pharmaceutical composition comprising a compound or salt of Formula I.

In another aspect, the invention encompasses the use of a therapeutically effective amount of a compound or salt of Formula I for the preparation of a medicament for the treatment of cancer, inflammation, or arthritis in a patient in need of such treatment.

In another aspect, the invention encompasses a package comprising a compound or salt of Formula I in a container with instructions on how to use the compound.

In another aspect, the invention encompasses the use of a therapeutically effective amount of a compound or salt according of Formula I for the preparation of a medicament for the treatment of a disease or condition related to cell proliferation in a patient in need of such treatment.

In another aspect, the invention encompasses the use of a therapeutically effective amount of a compound or salt according of Formula I for the preparation of a medicament for the treatment of a disease or condition related to cell proliferation in a patient in need of such treatment, wherein the disease or condition is cancer, inflammation, or arthritis.

In another aspect, the invention encompasses the use of therapeutically effective amount of a compound or salt of Formula I for the preparation of a medicament for the treatment of a disease or disorder related to the activity of heat shock protein 90, in a subject in need of such.

In another aspect, the invention encompasses the use of therapeutically effective amount of a compound or salt of Formula I, alone or in combination with another therapeutic agent, for the preparation of a medicament for the treatment of a disease or disorder related to the activity of heat shock protein 90 and/or its client proteins, in a subject in need of such, wherein the HSP-90 mediated disorder is selected from the group of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases and malignant disease.

In a preferred aspect, the invention encompasses methods for the treatment of cancer in a subject in need of such treatment comprising administration of therapeutically effective amount of a compound or salt of Formula I, in combination with at least one other therapeutic agent.

In a more preferred aspect, the invention encompasses methods for treating cancer in a subject in need of such treatment, the methods comprising administration of therapeutically effective amount of a compound or salt of Formula I, in combination with at least one other anti-cancer agent.

In another preferred aspect, the invention encompasses methods for treating cancer, the methods comprising administration, to a subject in need of such treatment, of a therapeutically effective amount of a compound or salt of Formula I, in combination with radiation therapy.

In another aspect, the invention encompasses the use of therapeutically effective amount of a compound or salt of Formula I for the preparation of a medicament for the treatment of a fibrogenetic disorder related to the activity of heat shock protein 90, in a subject in need of such,

wherein the fibrogenetic disorder is selected from the group of scleroderma, polymyositis, systemic lupus, rheumatoid arthritis, liver cirrhosis, keloid formation, interstitial nephritis and pulmonary fibrosis.

In another aspect, the invention encompasses the use of a therapeutically effective amount of a compound or salt of Formula I for the preparation of a medicament for protecting a subject from infection caused by an organism selected from Plasmodium species.

In a preferred aspect, the invention encompasses the use of a therapeutically effective amount of a compound or salt of Formula I for the preparation of a medicament for protecting a subject from infection caused by Plasmodium falciparum.

In another aspect, the invention encompasses the use of a therapeutically effective amount of a compound or salt of Formula I for the preparation of a medicament for reducing the level of infection caused by an organism selected from Plasmodium species in a subject in need of such treatment.

In a preferred aspect, the invention encompasses the use of a therapeutically effective amount of a compound or salt of Formula I for the preparation of a medicament for reducing the level of infection caused by Plasmodium falciparum in a subject in need of such treatment

In another aspect, the invention encompasses the use of a therapeutically effective amount of a compound or salt of Formula I for the preparation of a medicament for treating a patient infected with a metazoan parasite.

In a preferred aspect, the invention encompasses the use of a therapeutically effective amount of a compound or salt of Formula I for the preparation of a medicament for treating a patient infected by a metazoan parasite which is Plasmodium falciparum.

In another aspect, the invention encompasses the use of a therapeutically effective amount of a compound or salt of Formula I in combination with one or more known anti-fungal drugs for the preparation of a medicament for treating a patient infected with a fungal infection.

In the methods for treating viral infections, particular viral infections include those resulting from HIV-1 and Hepatitis C virus.

### DEFINITIONS

The term “alkoxy” represents an alkyl group of indicated number of carbon atoms attached to the parent molecular moiety through an oxygen bridge. Examples of alkoxy groups include, for example, methoxy, ethoxy, propoxy and isopropoxy.

As used herein, the term “alkyl” includes those alkyl groups of a designated number of carbon atoms. Alkyl groups may be straight, or branched. Examples of “alkyl” include methyl, ethyl, propyl, isopropyl, butyl, iso-, sec- and tert-butyl, pentyl, hexyl, heptyl, 3-ethylbutyl, and the like.

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenoxy” refers to an alkenyl group attached to the parent group through an oxygen atom.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl” refers to an aromatic hydrocarbon ring system containing at least one aromatic ring. The aromatic ring may optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl, naphthyl, and anthracenyl. More preferred aryl groups are phenyl and naphthyl. Most preferred is phenyl. The aryl groups of the invention may be substituted with various groups as provided herein. Thus, any carbon atom present within an aryl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₈heterocycloalkyl, C₁-C₈alkenyl, C₁-C₈alkynyl, halo(C₁-C₈)alkyl, halo(C₁-C₈)alkoxy, oxo, amino(C₁-C₈)alkyl, mono- and di(C₁-C₈alkyl)amino(C₁-C₈)alkyl, C₁-C₈acyl, C₁-C₈acyloxy, C₁-C₈sulfonyl, C₁-C₈thio, C₁-C₈sulfonamido, C₁-C₈aminosulfonyl.

The term “carboxy” as used herein, means a —CO₂H group.

The term “cycloalkyl” refers to a C₃-C₈cyclic hydrocarbon. Examples of cycloalkyl include cyclopropyl, cyclobutyl, cyclopentyl, cyclohexyl, cycloheptyl and cyclooctyl. More preferred are C₃-C₆ cycloalkyl groups. The cycloalkyl groups of the invention may be substituted with various groups as provided herein. Thus, any carbon atom present within a cycloalkyl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₈heterocycloalkyl, C₁-C₈alkenyl, C₁-C₈alkynyl, halo(C₁-C₈)alkyl, halo(C₁-C₈)alkoxy, oxo, amino(C₁-C₈)alkyl and mono- and di(C₁-C₈alkyl)amino(C₁-C₈)alkyl.

The terms “halogen” or “halo” indicate fluorine, chlorine, bromine, and iodine.

The term “haloalkoxy” refers to an alkoxy group substituted with one or more halogen atoms, where each halogen is independently F, Cl, Br or I. Preferred halogens are F and Cl. Preferred haloalkoxy groups contain 1-6 carbons, more preferably 1 to 4 carbons, and still more preferably 1-2 carbons. “Haloalkoxy” includes perhaloalkoxy groups, such as OCF₃ or OCF₂CF₃. A preferred haloalkoxy group is trifluoromethoxy.

The term “haloalkyl” refers to an alkyl group substituted with one or more halogen atoms, where each halogen is independently F, Cl, Br or I. Preferred halogens are F and Cl. Preferred haloalkyl groups contain 1-6 carbons, more preferably 1 to 4 carbons, and still more preferably 1-2 carbons. “Haloalkyl” includes perhaloalkyl groups, such as CF₃ or CF₂CF₃. A preferred haloalkyl group is trifluoromethyl.

The term “heterocycloalkyl” refers to a ring or ring system containing at least one heteroatom selected from nitrogen, oxygen, and sulfur, wherein said heteroatom is in a non-aromatic ring. The heterocycloalkyl ring is optionally fused to or otherwise attached to other heterocycloalkyl rings and/or non-aromatic hydrocarbon rings and/or phenyl rings. Preferred heterocycloalkyl groups have from 3 to 7 members. More preferred heterocycloalkyl groups have 5 or 6 members. Examples of heterocycloalkyl groups include, for example, 1,2,3,4-tetrahydroisoquinolinyl, piperazinyl, morpholinyl, piperidinyl, tetrahydrofuranyl, pyrrolidinyl, pyridinonyl, and pyrazolidinyl. Preferred heterocycloalkyl groups include piperidinyl, piperazinyl, morpholinyl, pyrrolidinyl, pyridinonyl, dihydropyrrolidinyl, and pyrrolidinonyl. The heterocycloalkyl groups of the invention may be substituted with various groups as provided herein. Thus, any atom present within a heterocycloalkyl ring and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₈heterocycloalkyl, C₁-C₈alkenyl, C₁-C₈alkynyl, halo(C₁-C₈) alkyl, halo(C₁-C₈) alkoxy, oxo, amino(C₁-C₈)alkyl and mono- and di(C₁-C₈alkyl)amino(C₁-C₈) alkyl.

The term “heteroaryl” refers to an aromatic ring system containing at least one heteroatom selected from nitrogen, oxygen, and sulfur. The heteroaryl ring may be fused or otherwise attached to one or more heteroaryl rings, aromatic or non-aromatic hydrocarbon rings or heterocycloalkyl rings. Examples of heteroaryl groups include, for example, pyridine, furan, thienyl, 5,6,7,8-tetrahydroisoquinoline and pyrimidines. The heteroaryl groups of the invention may be substituted with various groups as provided herein. Thus, any carbon atom present within an heteroaryl ring system and available for substitution may be further bonded to a variety of ring substituents, such as, for example, halogen, hydroxy, nitro, cyano, amino, C₁-C₈alkyl, C₁-C₈alkoxy, mono- and di(C₁-C₈alkyl)amino, C₃-C₁₀cycloalkyl, (C₃-C₁₀cycloalkyl)alkyl, (C₃-C₁₀cycloalkyl)alkoxy, C₂-C₈heterocycloalkyl, C₁-C₈alkenyl, C₁-C₈alkynyl, halo(C₁-C₈) alkyl, halo(C₁-C₈) alkoxy, oxo, amino(C₁-C₈) alkyl and mono- and di(C₁-C₈alkyl)amino(C₁-C₈) alkyl.

Preferred examples of heteroaryl groups include thienyl, benzothienyl, pyridyl, quinolyl, pyrazolyl, pyrimidyl, imidazolyl, benzimidazolyl, furanyl, benzofuranyl, dibenzofuranyl, thiazolyl, benzothiazolyl, isoxazolyl, oxadiazolyl, isothiazolyl, benzisothiazolyl, triazolyl, pyrrolyl, indolyl, pyrazolyl, and benzopyrazolyl.

The compounds of this invention may contain one or more asymmetric carbon atoms, so that the compounds can exist in different stereoisomeric forms. These compounds can be, for example, racemates, chiral non-racemic or diastereomers. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

When the compounds described herein contain olefinic double bonds or other centers of geometric asymmetry, and unless otherwise specified, it is intended that the compounds include the cis, trans, Z- and E-configurations. Likewise, all tautomeric forms are also intended to be included.

### Pharmaceutical Compositions

The compounds of general Formula I may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound of general Formula I and a pharmaceutically acceptable carrier. One or more compounds of general Formula I may be present in association with one or more non-toxic pharmaceutically acceptable carriers and/or diluents and/or adjuvants, and if desired other active ingredients. The pharmaceutical compositions containing compounds of general Formula I may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of general Formula I may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds of general Formula I may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For disorders of the eye or other external tissues, e.g., mouth and skin, the formulations are preferably applied as a topical gel, spray, ointment or cream, or as a suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The antiinflammatory active ingredients are preferably present in such formulations in a concentration of 0.5 to 20%, advantageously 0.5 to 10% and particularly about 1.5% w/w. For therapeutic purposes, the active compounds of this combination invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. The daily dose can be administered in one to four doses per day. In the case of skin conditions, it may be preferable to apply a topical preparation of compounds of this invention to the affected area two to four times a day.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water. Preferred non-human animals include domesticated animals.

The compounds of the present invention may be administered alone or in combination with at least one additional therapeutic agent or therapy, e.g., radiation therapy, to a patient in need of such treatment. The additional therapeutic agent or therapy may be administered at the same time, separately, or sequentially with respect to the administration of a compound of the invention. Such additional therapeutic agents included, but are not limited to, anti-cancer agents, anti-inflammatory agents, and the like.

The compounds of the present invention may be prepared by use of known chemical reactions and procedures. Representative methods for synthesizing compounds of the invention are presented below. It is understood that the nature of the substituents required for the desired target compound often determines the preferred method of synthesis. All variable groups of these methods are as described in the generic description if they are not specifically defined below.

### Methods of Preparation

### General Procedure

Representative synthetic procedures for the preparation of compounds of the invention are outlined below in following schemes. Unless otherwise indicated, all variables carry the definitions set forth above in connection with Formula I.

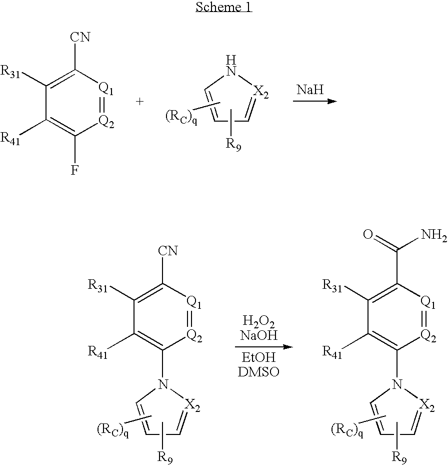

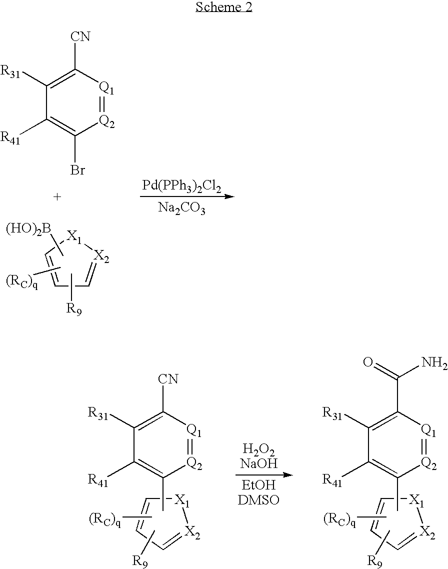

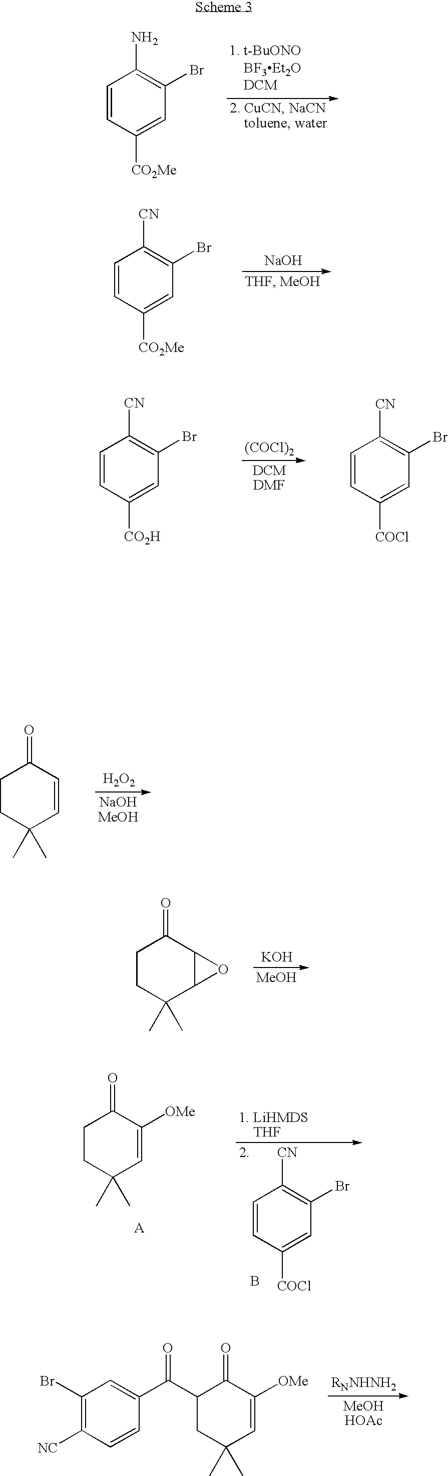

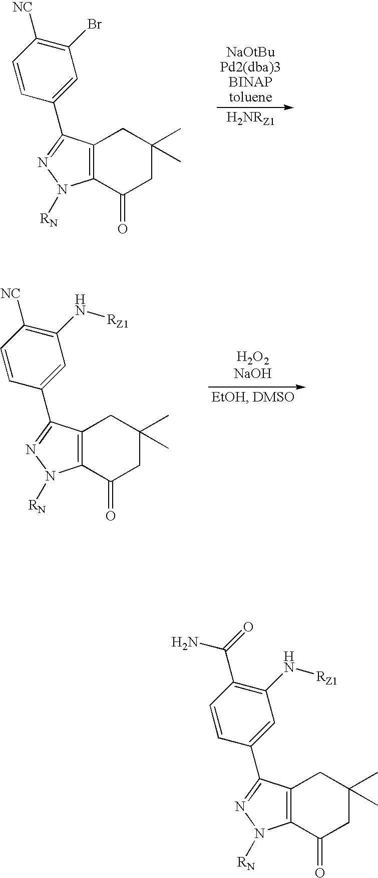

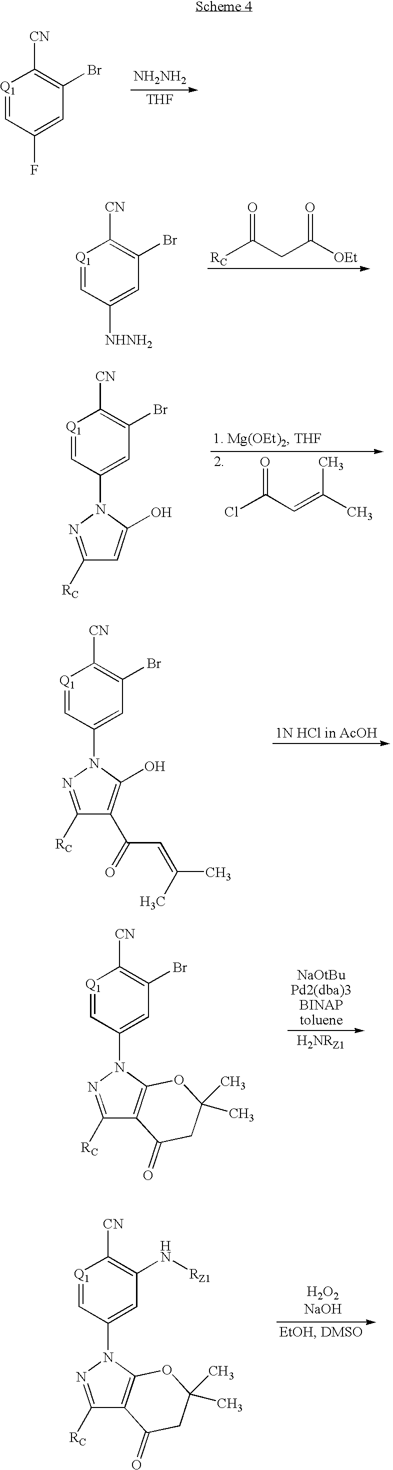

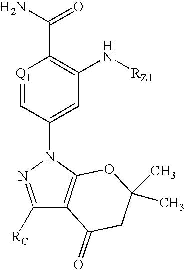

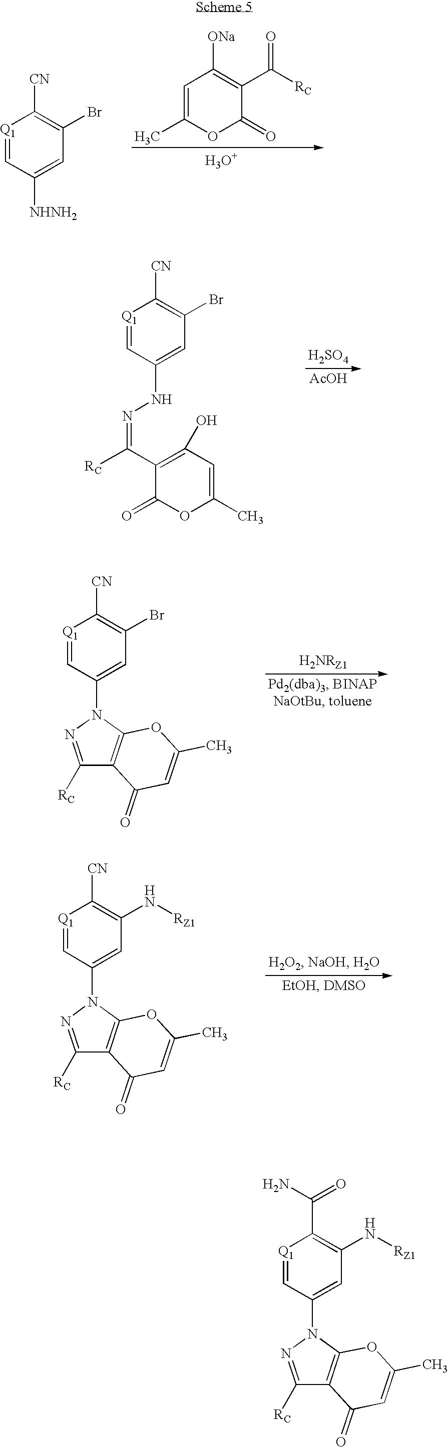

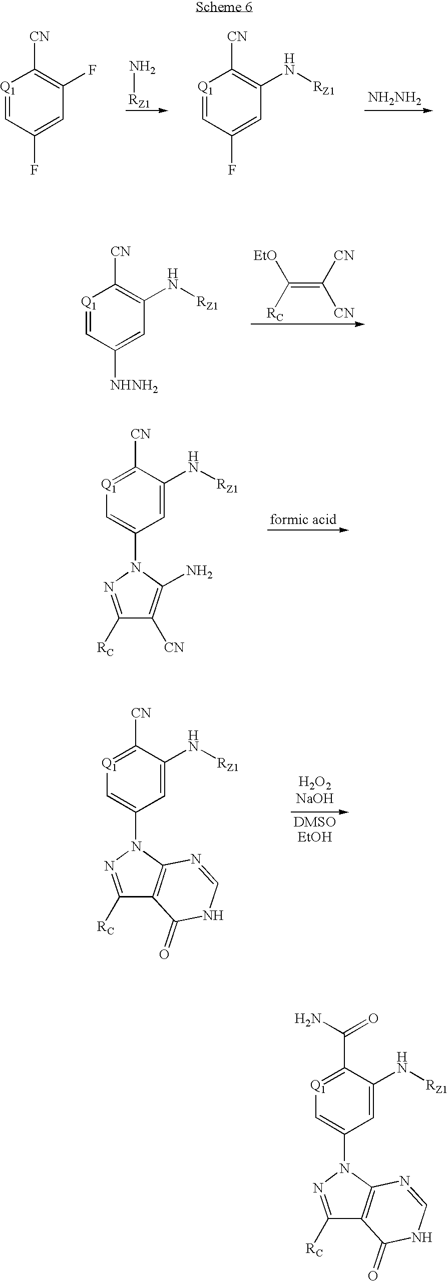

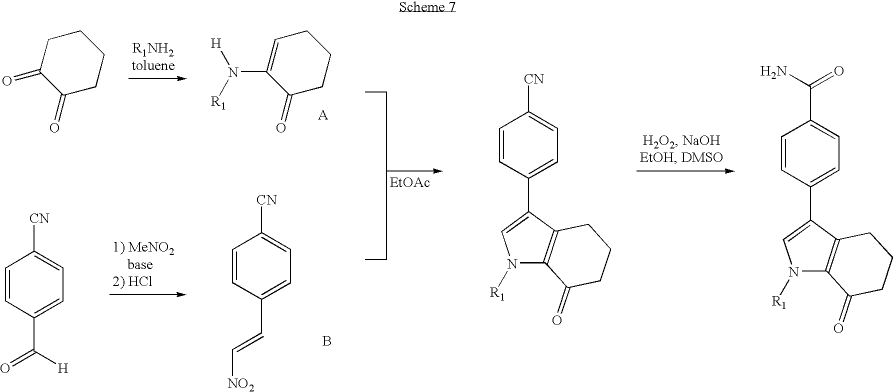

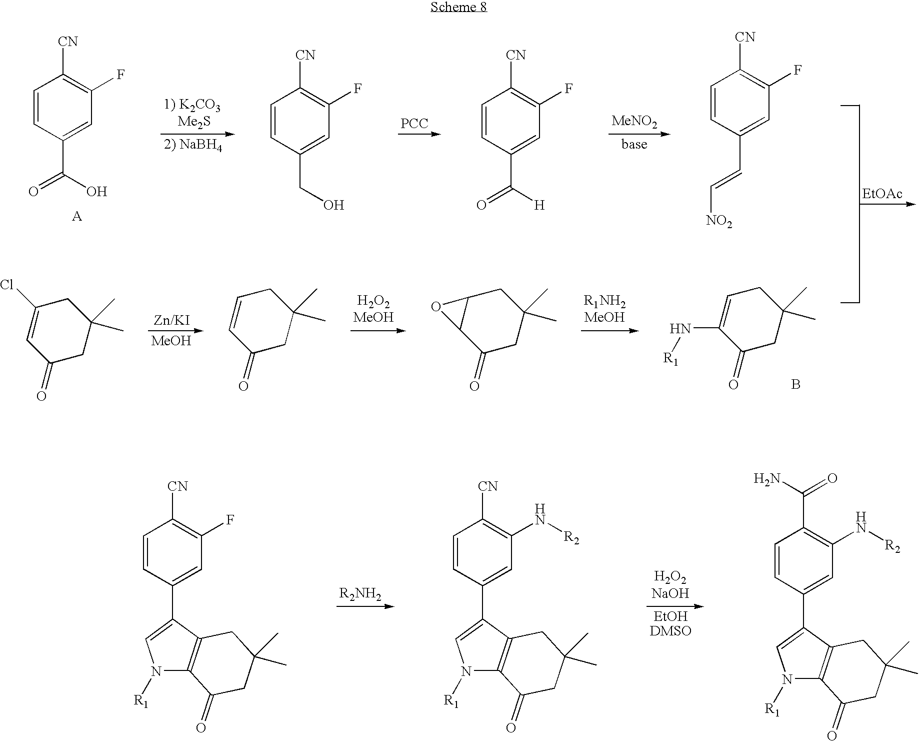

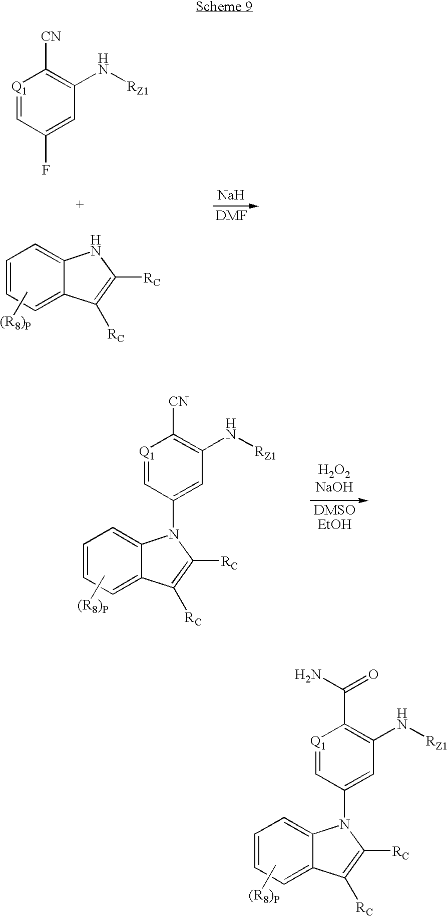

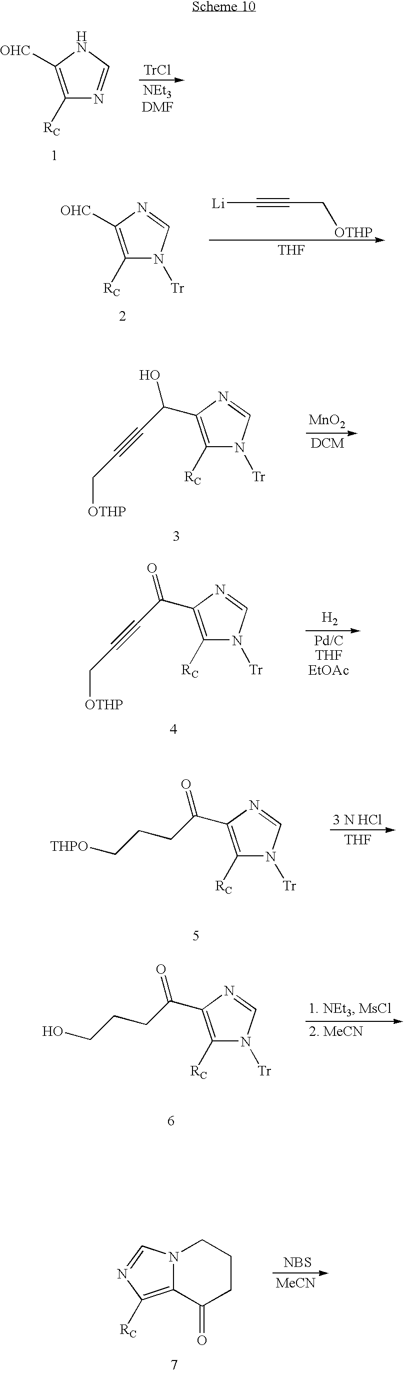

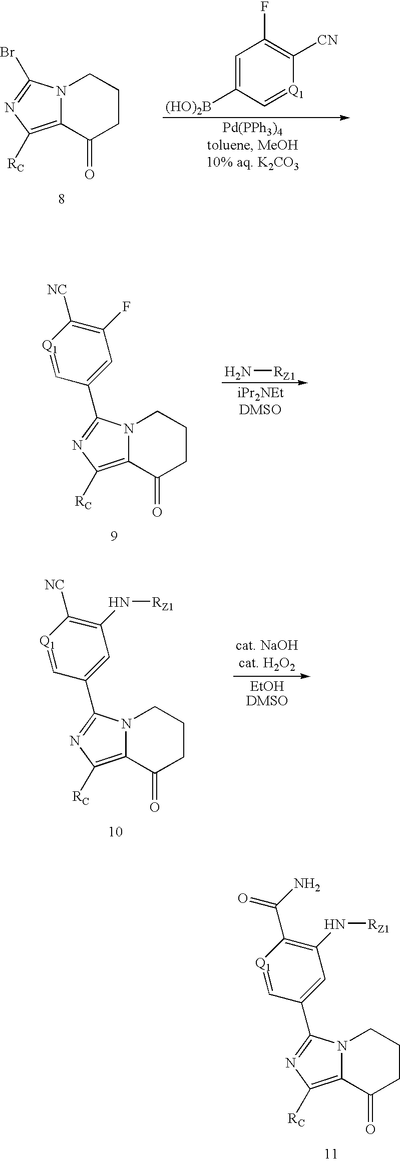

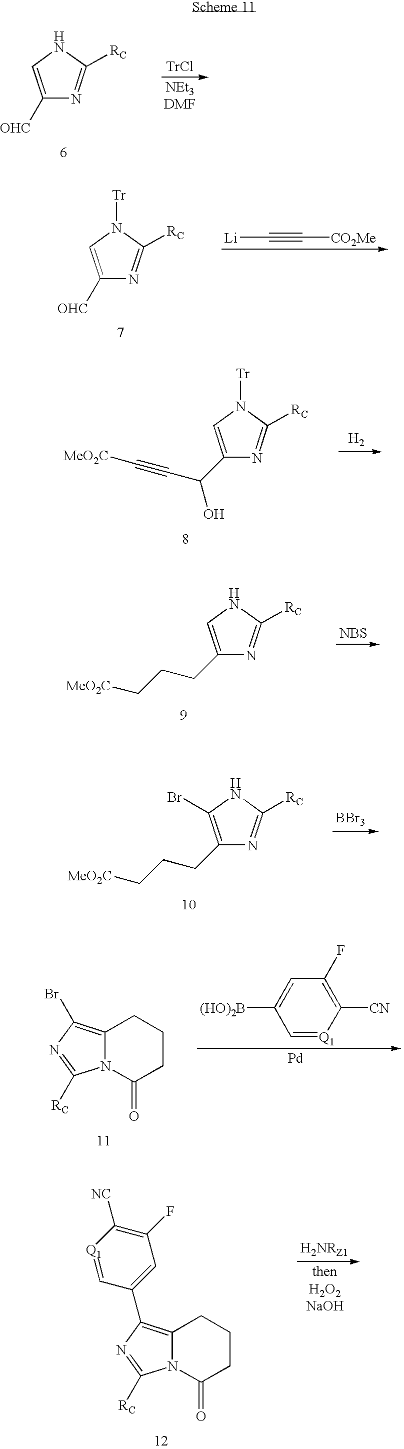

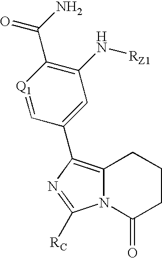

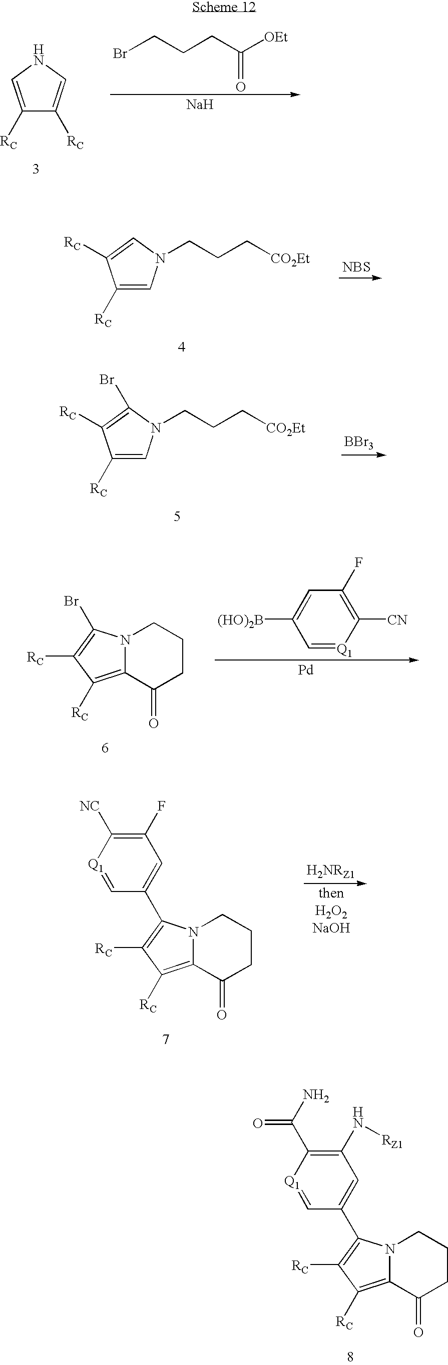

Those having skill in the art will recognize that the starting materials and reaction conditions may be varied, the sequence of the reactions altered, and additional steps employed to produce compounds encompassed by the present invention, as demonstrated by the following examples. In some cases, protection of certain reactive functionalities may be necessary to achieve some of the above transformations. In general, the need for such protecting groups as well as the conditions necessary to attach and remove such groups will be apparent to those skilled in the art of organic synthesis.

The disclosures of all articles and references mentioned in this application, including patents, are incorporated herein by reference in their entirety.

### EXAMPLES

The preparation of the compounds of the invention is illustrated further by the following examples, which are not to be construed as limiting the invention in scope or spirit to the specific procedures and compounds described in them.

In all cases, unless otherwise specified, the column chromatography is performed using a silica gel solid phase.

### Example 1

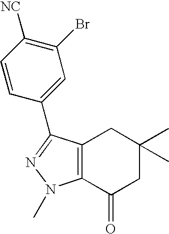

### 2-Bromo-4-(1,5,5-trimethyl-7-oxo-4,5,6,7-tetrahydro-1H-indazol-3-yl)-benzonitrile

### Compound 1

### Example 1a

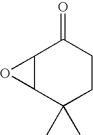

### Preparation of 5,5-Dimethyl-7-oxa-bicyclo[4.1.0]heptan-2-one

To an ice-cold solution of 4,4-Dimethyl-cyclohex-2-enone (1.00 g, 8.05 mmol) in methanol (8 mL) is added 35% hydrogen peroxide (3.5 mL, 40.86 mmol) followed by 0.5 N NaOH (2.2 mL, 1.1 mmol). The mixture is stirred at 0° C. for 1 h, stored in a freezer (−15° C.) overnight and then stirred at 0° C. for another 6 h. After this time, water (15 mL) is added, and the mixture is extracted with dichloromethane (9×30 mL). The organic layers are combined, washed with 10% Na₂SO₃ (2×40 mL) and brine, dried over Na₂SO₄, filtered and concentrated at reduced pressure to afford 5,5-Dimethyl-7-oxa-bicyclo[4.1.0]heptan-2-one (1.04 g, 92%) as a colorless oil.

### Example 1b

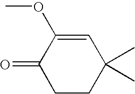

### Preparation of 2-Methoxy-4,4-dimethyl-cyclohex-2-enone

To a solution of KOH (0.49 g, 7.4 mmol) in methanol (15 mL) is added a solution of 5-Dimethyl-7-oxa-bicyclo[4.1.0]heptan-2-one (1.04 g, 7.42 mmol) in methanol (5 mL). The mixture is stirred overnight at room temperature and then heated at reflux for 20 min. After cooling to room temperature, water (40 mL) is added, and the mixture is extracted with diethyl ether (3×20 mL). The organics are combined, washed with brine, dried over Na₂SO₄, filtered, and concentrated at reduced pressure. The residue is stirred with hexanes for 30 min. The resulting solids are removed by filtration, and the filtrate is concentrated at reduced pressure to afford 2-Methoxy-4,4-dimethyl-cyclohex-2-enone (0.47 g, 41%) as an oil that solidified on standing.

### Example 1c

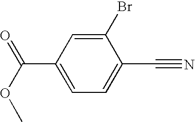

### Preparation of 3-Bromo-4-cyano-benzoic acid methyl ester

A solution of 4-Amino-3-bromo-benzoic acid methyl ester (1.00 g, 4.35 mmol) in dichloromethane (20 mL) is cooled to −10° C. and treated dropwise with 90% tert-butyl nitrite (0.58 g, 0.67 mL, 5.08 mmol) followed by boron trifluoride diethyl etherate (0.94 g, 0.81 mL, 6.62 mmol). The suspension is allowed to warm to room temperature and stirred for 4 h. Diethyl ether (20 mL) is added, and the off-white solid is collected by filtration, washed with diethyl ether and dried briefly under high vacuum. The solid is then suspended in toluene (15 mL) and cooled to 0° C. A solution of copper (I) cyanide (0.52 g, 5.8 mmol) and sodium cyanide (0.71 g, 14.5 mmol) in water (10 mL) is added dropwise over 10 min. The mixture is stirred at 0° C. for 30 min, allowed to warm to room temperature, and then heated to 60° C. After 1 h, the solids are completely dissolved. The mixture is allowed to cool to room temperature, and ethyl acetate (20 mL) and water (20 mL) are added. The aqueous layer is separated and extracted with ethyl acetate (2×20 mL). The organic layers are combined, washed with water (2×10 mL) and brine, dried over Na₂SO₄, filtered and concentrated at reduced pressure. The solid residue obtained is recrystallized from hexanes/ethyl acetate to afford 3-Bromo-4-cyano-benzoic acid methyl ester (0.42 g, 39%) as a tan solid. The recrystallization mother liquor is concentrated and chromatographed (Biotage, 95:5 to 85:15 hexanes/ethyl acetate) to afford an additional portion of 3-Bromo-4-cyano-benzoic acid methyl ester (0.34 g, total yield: 0.76 g, 73%).

### Example 1d

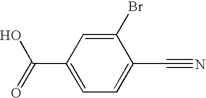

### Preparation of 3-Bromo-4-cyano-benzoic acid

A 2 N NaOH solution (2 mL) is added to a solution of 3-Bromo-4-cyano-benzoic acid methyl ester (0.41 g, 1.7 mmol) in THF (10 mL) and methanol (5 mL). After 2 h of stirring at room temperature, the volatiles are removed at reduced pressure. The residue obtained is diluted with water (5 mL) and acidified to pH 3-4 with 2 N HCl. The resulting off-white solid is collected by filtration and dried under vacuum to afford 3-Bromo-4-cyano-benzoic acid (0.35 g, 89%): ESI MS m/z 224 [M−H]⁻.

### Example 1e

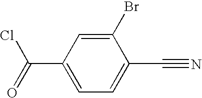

### Preparation of 3-Bromo-4-cyano-benzoyl chloride

Oxalyl chloride (0.22 g, 0.15 mL, 1.7 mmol) is added dropwise to an ice-cooled solution of acid 3-Bromo-4-cyano-benzoic acid (0.25 g, 1.1 mmol) in dichloromethane (10 mL) and DMF (10 drops). The mixture is allowed to stir at 0° C. for 5 min and at room temperature for 30 min. The solvent is then removed at reduced pressure, and the residue is azeotroped with dichloromethane (2×3 mL) and dried under high vacuum for 1 h to afford 3-Bromo-4-cyano-benzoyl chloride as a white solid which is used directly in the next step.

### Example 1f

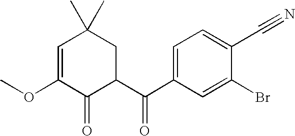

### Preparation of 2-Bromo-4-(3-methoxy-5,5-dimethyl-2-oxo-cyclohex-3-enecarbonyl)-benzonitrile

To a solution of 1 M lithium bis(trimethylsilyl)amide in THF (0.98 mL, 0.98 mmol), diluted with THF (15 mL) and cooled to −20° C., is added dropwise a solution of 2-Methoxy-4,4-dimethyl-cyclohex-2-enone (0.15 g, 0.96 mmol) in THF (5 mL). The solution is allowed to warm to room temperature, stirred for 45 min, and then cooled to −78° C. A suspension of acid chloride 3-Bromo-4-cyano-benzoyl chloride (est. 1.09 mmol) in THF (5 mL) is added. After stirring at −78° C. for 1 h, a 10% solution of NaH₂PO₄ is added. The mixture is extracted with ethyl acetate (3×20 mL), and the organics are combined, washed with brine, dried over Na₂SO₄, filtered and concentrated at reduced pressure. Flash chromatography (silica gel, 90:10 to 85:15 hexanes/ethyl acetate) gave 2-Bromo-4-(3-methoxy-5,5-dimethyl-2-oxo-cyclohex-3-enecarbonyl)-benzonitrile (0.16 g, 46%) as a yellow solid: APCI m/z 360 [M−H]⁻.

### Example 1g

### Preparation of 2-Bromo-4-(1,5,5-trimethyl-7-oxo-4,5,6,7-tetrahydro-1H-indazol-3-yl)-benzonitrile

Acetic acid (2 drops) is added to a stirred mixture of 2-Bromo-4-(3-methoxy-5,5-dimethyl-2-oxo-cyclohex-3-enecarbonyl)-benzonitrile (0.077 g, 0.18 mmol) and methylhydrazine (0.011 g, 0.23 mmol) in methanol (5 mL). The mixture is heated at reflux for 1 h, then an additional portion of methylhydrazine (0.003-0.004 g in MeOH) is added and reflux is continued. After 30 min, the mixture is cooled and concentrated at reduced pressure. The residue obtained is dissolved in THF (3 mL) and stirred for 30 min with 1 N HCl (1 mL). The mixture is neutralized by adding to saturated aqueous NaHCO₃ and then extracted with ethyl acetate (3×20 mL). The organics are combined, washed with brine, dried over Na₂SO₄, filtered and concentrated at reduced pressure. Chromatography (Biotage, 95:5 to 70:30 hexanes/ethyl acetate) gave 2-Bromo-4-(1,5,5-trimethyl-7-oxo-4,5,6,7-tetrahydro-1H-indazol-3-yl)-benzonitrile (0.024 g, 32%) as a white solid: ESI MS m/z 358 [M+H]⁺.

### Example 2

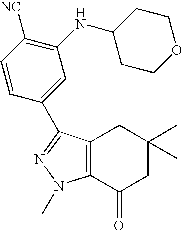

### 2-(Tetrahydro-pyran-4-ylamino)-4-(1,5,5-trimethyl-7-oxo-4,5,6,7-tetrahydro-1H-indazol-3-yl)-benzo-nitrile

### Compound 2

Sodium tert-butoxide (0.013 g, 0.14 mmol) is added to a solution of 2-Bromo-4-(1,5,5-trimethyl-7-oxo-4,5,6,7-tetrahydro-1H-indazol-3-yl)-benzonitrile (0.024 g, 0.067 mmol) and 4-aminotetrahydropyran (0.011 g, 0.11 mmol) in toluene (2 mL). The mixture is degassed with three vacuum/argon backfill cycles. rac-BINAP (0.004 g, 0.0064 mmol) and tris(dibenzylideneacetone)dipalladium(0) (0.004 g, 0.004 mmol) are added, and the degas cycle is repeated two more times. The mixture is then heated at 80° C. for 4 h. After cooling to room temperature, the solvent is removed at reduced pressure, and the residue obtained is chromatographed (silica gel flash column, 85:15 to 75:25 hexanes/ethyl acetate) to afford 2-(Tetrahydro-pyran-4-ylamino)-4-(1,5,5-trimethyl-7-oxo-4,5,6,7-tetrahydro-1H-indazol-3-yl)-benzonitrile (0.024 g, 99%) as a yellow solid: APCI MS m/z 379 [M+H]⁺.

### Example 3

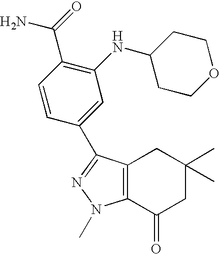

### 2-(Tetrahydro-pyran-4-ylamino)-4-(1,5,5-trimethyl-7-oxo-4,5,6,7-tetrahydro-1H-indazol-3-yl)-benzamide

### Compound 3

A 2 N NaOH solution (2 drops) and 3% hydrogen peroxide (1 drop) are added to a stirred suspension of 2-(Tetrahydro-pyran-4-ylamino)-4-(1,5,5-trimethyl-7-oxo-4,5,6,7-tetrahydro-1H-indazol-3-yl)-benzonitrile (0.024 g, 0.067 mmol) in ethanol (2 mL) and DMSO (0.5 mL). The mixture is stirred at room temperature for 30 min. After this time, TLC analysis indicated the reaction is not complete, so additional 2 N NaOH (2 drops) and hydrogen peroxide (2 drop) are added. After another 1 h, the mixture is diluted with water (5 mL) and ethyl acetate (5 mL). The aqueous layer is separated and extracted with ethyl acetate (3×15 mL). The organic layers are combined, washed with 10% aqueous Na₂SO₃ (10 mL) and brine (2×10 mL), dried over Na₂SO₄, filtered and concentrated at reduced pressure. The residue obtained is combined with a smaller batch of crude product. Chromatography (silica gel flash column, 70:30 to 33:67 hexanes/ethyl acetate) gave 2-(Tetrahydro-pyran-4-ylamino)-4-(1,5,5-trimethyl-7-oxo-4,5,6,7-tetrahydro-1H-indazol-3-yl)-benzamide (0.033 g, 78%) as a white solid: ESI MS m/z 397 [M+H]⁺.

### Example 4

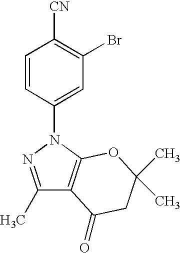

### 2-Bromo-4-(3,6,6-trimethyl-4-oxo-5,6-dihydro-4H-pyrano[2,3-c]pyrazol-1-yl)-benzonitrile

### Compound 4

### Example 4a

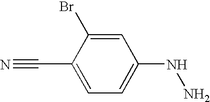

### Preparation of 2-Bromo-4-hydrazino-benzonitrile

To a solution of 2-bromo-4-fluorobenzonitrile (5.00 g, 25.00 mmol) in THF (10 mL) is added anhydrous hydrazine (10 mL) through an addition funnel. After addition, the resulting mixture is stirred at room temperature for 24 h. Water (20 mL) is added to the reaction mixture. A white precipitate is collected by filtration, washed with water, and dried in a vacuum oven at 40° C. to afford 2-Bromo-4-hydrazino-benzonitrile (4.92 g, 93%) as a white solid: ¹H NMR (300 MHz, CDCl₃) δ8.05 (br s, 1H), 7.47 (d, J=8.7 Hz, 1H), 7.06 (d, J=2.0 Hz, 1H), 6.72 (dd, J=8.7, 2.0 Hz, 1H), 4.37 (br s, 2H); ESI MS m/z 212 [M+H]⁺.

### Example 4b

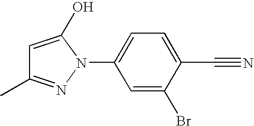

### Preparation of 2-Bromo-4-(5-hydroxy-3-methyl-pyrazol-1-yl)-benzonitrile

A suspension of 2-Bromo-4-hydrazino-benzonitrile (2.12 g, 10.00 mmol) and 3-Oxo-butyric acid ethyl ester (1.26 mL) in acetic acid (8 mL) is stirred and heated at reflux for 26 h. After cooling to room temperature, the precipitate is collected by filtration, washed with water, and dried in a vacuum oven at 40° C. to afford 2-Bromo-4-(5-hydroxy-3-methyl-pyrazol-1-yl)-benzonitrile (2.57 g, 92%) as an off-white solid: ¹H NMR (300 MHz, DMSO-d₆) δ 8.26-7.95 (m, 4H), 5.42 (br s, 1H), 2.13 (s, 3H); ESI MS m/z 278 [M+H]⁺.

### Example 4c

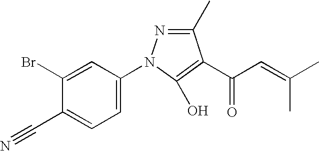

### Preparation of 2-Bromo-4-[5-hydroxy-3-methyl-4-(3-methyl-but-2-enoyl)-pyrazol-1-yl]-benzonitrile

A mixture of 2-Bromo-4-(5-hydroxy-3-methyl-pyrazol-1-yl)-benzonitrile (0.56 g, 2.0 mmol) and magnesium ethoxide (0.30 g, 2.0 mmol) in anhydrous THF (10 mL) is reflux for 4 h. After this reaction mixture is cooled to 0-5° C., a solution of 3-Methyl-but-2-enoyl chloride (0.25 mL, 2.0 mmol) in anhydrous THF (5 mL) is added dropwise. After stirring at room temperature overnight, this reaction mixture is poured into 1N HCl ice-water (50 mL) and stirred for 15 min. The precipitate is collected, washed with water and dried to affords 2-Bromo-4-[5-hydroxy-3-methyl-4-(3-methyl-but-2-enoyl)-pyrazol-1-yl]-benzonitrile (0.72 mg, >99%) as a light yellow solid: ¹H NMR (300 MHz, DMSO-d₆) δ 8.40 (d, J=1.5 Hz, 1H), 8.09-7.96 (m, 3H), 6.90 (s, 1H), 2.40 (s, 3H), 2.15 (s, 3H), 1.95 (s, 3H); ESI MS m/z 360 [M+H]⁺.

### Example 4d

### Preparation of 2-Bromo-4-(3,6,6-trimethyl-4-oxo-5,6-dihydro-4H-pyrano[2,3-c]pyrazol-1-yl)-benzonitrile

A mixture of 2-Bromo-4-[5-hydroxy-3-methyl-4-(3-methyl-but-2-enoyl)-pyrazol-1-yl]-benzonitrile (0.03 g, 0.08 mmol) in 1N HCl in acetic acid (1.0 mL) is heated to 60° C. for 0.5 h. After cooling to room temperature, the resulting mixture is added to ice-water (10 mL) dropwise. The solid is collected by filtration and washed with water. After drying, the solid is purified by column chromatography (silica gel, 7:3 hexane/ethyl acetate) to afford 2-Bromo-4-(3,6,6-trimethyl-4-oxo-5,6-dihydro-4H-pyrano[2,3-c]pyrazol-1-yl)-benzonitrile (0.021 g, 73%) as an off-white solid: ¹H NMR (300 MHz, CDCl₃) δ 8.25 (d, J=2.1 Hz, 1H), 7.93 (dd, J=8.6, 2.1 Hz, 1H), 7.71 (d, J=8.6 Hz, 1H), 2.65 (s, 2H), 2.47 (s, 3H), 1.63 s, 6H); ESI MS m/z 360 [M+H]⁺.

### Example 5

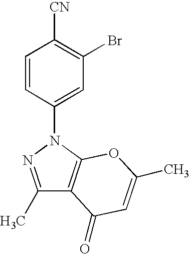

### 2-Bromo-4-(3,6-dimethyl-4-oxo-4H-pyrano[2,3-c]pyrazol-1-yl)-benzonitrile

### Compound 5

### Example 5a

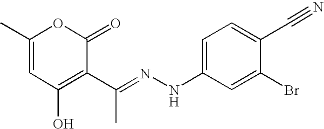

### Preparation of 2-Bromo-4-{N′-[1-(4-hydroxy-6-methyl-2-oxo-2H-pyran-3-yl)-ethylidene]-hydrazino}-benzonitrile

To a suspension of 2-Bromo-4-hydrazino-benzonitrile (1.10 g, 5.2 mmol) and dehydroacetic acid sodium salt (0.99 g, 5.2 mmol) in toluene (20 mL) is added 4 N HCl in dioxane (1.3 mL, 5.2 mmol). The resulting mixture is stirred at 80° C. for 15 h. After cooling to room temperature, the precipitate is collected by filtration, washed with 1:1 ethanol/water and dried to afford 2-Bromo-4-{N′-[1-(4-hydroxy-6-methyl-2-oxo-2H-pyran-3-yl)-ethylidene]-hydrazino}-benzonitrile (1.74 g, 92%) as a pale yellow solid: ¹H NMR (300 MHz, DMSO-d₆) δ10.03 (br s, 1H), 7.74 (d, J=8.7 Hz, 1H), 7.27 (d, J=2.1 Hz, 1H), 7.00 (dd, J=8.7, 2.1 Hz, 1H), 6.06 (d, J=0.69 Hz, 1H), 2.42 (s, 3H), 2.19 (s, 3H).

### Example 5b

### Preparation of 2-Bromo-4-(3,6-dimethyl-4-oxo-4H-pyrano[2,3-c]pyrazol-1-yl)-benzonitrile

A mixture of 2-Bromo-4-{N′-[1-(4-hydroxy-6-methyl-2-oxo-2H-pyran-3-yl)-ethylidene]-hydrazino}-benzonitrile (1.00 g, 2.76 mmol) and concentrated H₂SO₄ (2 drops) in acetic acid (15 mL) is stirred at 110° C. for 2.5 h. After cooling to room temperature, the reaction mixture is poured into ice-water (50 mL). The resulting precipitate is collected by filtration, and washed with water, saturated aqueous NaHCO₃ solution, water again, and dried. The solid is purified by column chromatography (silica gel, 7:3 ethyl acetate/hexane) to afford 2-Bromo-4-(3,6-dimethyl-4-oxo-4H-pyrano[2,3-c]pyrazol-1-yl)-benzonitrile (0.18 g, 19%) as an off-white solid: ESI MS m/z 345 [M+H]⁺.

### Example 6

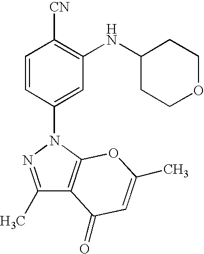

### 4-(3,6-Dimethyl-4-oxo-4H-pyrano[2,3-c]pyrazol-1-yl)-2-(tetrahydro-pyran-4-ylamino)-benzonitrile

### Compound 6

A mixture of 2-Bromo-4-(3,6-dimethyl-4-oxo-4H-pyrano[2,3-c]pyrazol-1-yl)-benzonitrile (0.08 g, 0.23 mmol), 4-aminotetrahydropyran (0.047 g, 0.46 mmol) and sodium tert-butoxide (0.044 g, 0.46 mmol) in anhydrous toluene (1.5 mL) is bubbled with N₂ for 5 min. Pd₂(dba)₃ (0.011 g, 0.012 mmol) and rac-BINAP (0.012 g, 0.019 mmol) are then added, and the mixture is stirred at 115° C. for 1 h. The reaction mixture is then cooled to room temperature, and purified by column chromatography (silica gel, 1:1 ethyl acetate/hexane) to afford 4-(3,6-Dimethyl-4-oxo-4H-pyrano[2,3-c]pyrazol-1-yl)-2-(tetrahydro-pyran-4-ylamino)-benzonitrile (0.019 mg, 23%) as a light brown solid: ESI MS m/z 365 [M+H]⁺.

### Example 7

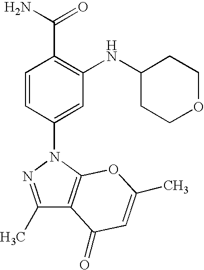

### 4-(3,6-Dimethyl-4-oxo-4H-pyrano[2,3-c]pyrazol-1-yl)-2-(tetrahydro-pyran-4-ylamino)-benzamide

### Compound 7

To a solution of 4-(3,6-Dimethyl-4-oxo-4H-pyrano[2,3-c]pyrazol-1-yl)-2-(tetrahydro-pyran-4-ylamino)-benzonitrile (0.024 g, 0.066 mmol) in ethanol/DMSO (3:1, 3.0 mL) is added water (6 drops), hydrogen peroxide solution (2 drops, 3.5% H₂O₂) and 1N sodium hydroxide solution (2 drops). After stirring at room temperature for 20 min, the resulting mixture is diluted with water (10 mL) and extracted with ethyl acetate (3×10 mL). The combined organic layer is dried over Na₂SO₄, filtered and concentrated at reduced pressure to dryness. The residue obtained is purified by preparative TLC (ethyl acetate) to afford 4-(3,6-Dimethyl-4-oxo-4H-pyrano[2,3-c]pyrazol-1-yl)-2-(tetrahydro-pyran-4-ylamino)-benzamide (0.003 g, 0.8%) as an off-white solid: APCI MS m/z 381 [M−H]⁻.

### Example 8

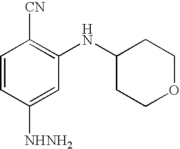

### 4-Hydrazino-2-(tetrahydro-pyran-4-ylamino)-benzonitrile

### Example 8a

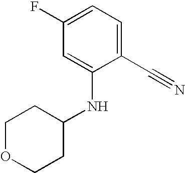

### Preparation of 4-Fluoro-2-(tetrahydro-pyran-4-ylamino)-benzonitrile

A mixture of 2,4-difluorobenzonitrile (0.420 g, 3.0 mmol), 4-aminotetrahydropyran (0.306 g, 3.0 mmol) and DIPEA (0.523 mL, 3.0 mmol) in DMSO (5.0 mL) is stirred at 150° C. (preheated oil-bath) for 20 min. After cooling to room temperature, the resulting mixture is poured into saturated aqueous NH₄Cl solution and extracted with ethyl acetate (3×20 mL). The combined organic layer is washed with brine (3×15 mL) and dried over Na₂SO₄, filtered and concentrated at reduced pressure to dryness. The residue obtained is purified by column chromatography (silica gel, 40:60 ethyl acetate/hexane) to afford 4-Fluoro-2-(tetrahydro-pyran-4-ylamino)-benzonitrile (0.214 g, 32%) as a pale yellow solid: ESI MS m/z 221 [M+H]⁺.

### Example 8b

### Preparation of 4-Hydrazino-2-(tetrahydro-pyran-4-ylamino)-benzonitrile

A mixture of 4-Fluoro-2-(tetrahydro-pyran-4-ylamino)-benzonitrile (0.224 g, 1.02 mmol) and hydrazine (2.0 mL) in 1,4-dioxane (2.0 mL) is stirred at 100° C. for 1 h. After cooling to room temperature, the reaction mixture is poured into saturated aqueous NaHCO₃ solution (20 mL) and extracted with ethyl acetate (3×15 mL). The combined organic layer is dried over Na₂SO₄, filtered and concentrated at reduced pressure to dryness to afford 4-Hydrazino-2-(tetrahydro-pyran-4-ylamino)-benzonitrile (0.221 g, 93%) as a white solid: ESI MS m/z 233 [M+H]⁺.

### Example 9

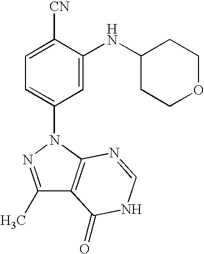

### 4-(3-methyl-4-oxo-4,5-dihydro-1H-pyrazolo[3,4-d]pyrimidin-1-yl)-2-(tetrahydro-2H-pyran-4-ylamino)benzonitrile

### Compound 8

### Example 9a

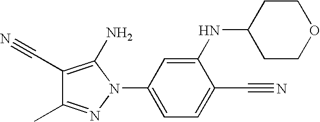

### Preparation of 5-Amino-1-[4-cyano-3-(tetrahydro-pyran-4-ylamino)-phenyl]-3-methyl-1H-pyrazole-4-carbonitrile

A mixture of 4-Hydrazino-2-(tetrahydro-pyran-4-ylamino)-benzonitrile (0.118 g, 0.51 mmol) and (1-ethoxyethylidene)malononitrile (0.076 g, 0.51 mmol) in ethanol (3.0 mL) is reflux for 5 h. The reaction mixture is evaporated to dryness. The residue obtained is purified by column chromatography (silica gel, 5:3:2 hexane/ethyl acetate/DCM) to afford 5-Amino-1-[4-cyano-3-(tetrahydro-pyran-4-ylamino)-phenyl]-3-methyl-1H-pyrazole-4-carbonitrile (0.083 g, 58%) as a light orange solid: ESI MS m/z 323 [M+H]⁺.

### Example 9b

### Preparation of 4-(3-Methyl-4-oxo-4,5-dihydro-pyrazolo[3,4-d]pyrimidin-1-yl)-2-(tetrahydro-pyran-4-ylamino)-benzonitrile

A mixture of 5-Amino-1-[4-cyano-3-(tetrahydro-pyran-4-ylamino)-phenyl]-3-methyl-1H-pyrazole-4-carbonitrile (0.072 g, 0.22 mmol) in formic acid (2.0 mL) is stirred at 100° C. for 7 h. After cooling to room temperature, the precipitate is collected by filtration and purified by column chromatography (silica gel, 95:5 DCM/methanol) to afford 4-(3-Methyl-4-oxo-4,5-dihydro-pyrazolo[3,4-d]pyrimidin-1-yl)-2-(tetrahydro-pyran-4-ylamino)-benzonitrile (0.013 g, 17%) as a white solid: ESI MS m/z 351 [M+H]⁺.

### Example 10

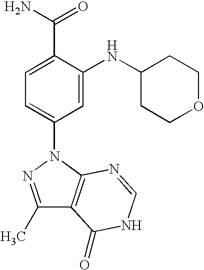

### 4-(3-Methyl-4-oxo-4,5-dihydro-pyrazolo[3,4-d]pyrimidin-1-yl)-2-(tetrahydro-pyran-4-ylamino)-benzamide

### Compound 9

To a suspension of 4-(3-Methyl-4-oxo-4,5-dihydro-pyrazolo[3,4-d]pyrimidin-1-yl)-2-(tetrahydro-pyran-4-ylamino)-benzonitrile (0.013 g, 0.004 mmol) in ethanol/DMSO (3:1, 2.0 mL) is added water (5 drops), hydrogen peroxide solution (2 drops, 3.5% H₂O₂) and 1N sodium hydroxide solution (2 drops). After stirring at room temperature for 15 min, the resulting mixture is diluted with brine (20 mL) and extracted with ethyl acetate (3×10 mL). The combined organic layer is concentrated at reduced pressure to dryness. The residue obtained is triturated with water and dried to afford 4-(3-Methyl-4-oxo-4,5-dihydro-pyrazolo[3,4-d]pyrimidin-1-yl)-2-(tetrahydro-pyran-4-ylamino)-benzamide (0.013 g, 94%) as a pale yellow solid: ESI MS m/z 369 [M+H]⁺.

### Example 11

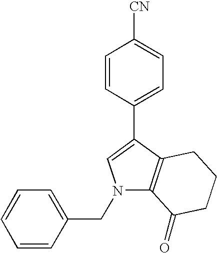

### 4-(1-Benzyl-7-oxo-4,5,6,7-tetrahydro-1H-indol-3-yl)-benzonitrile

### Compound 10

### Example 11a

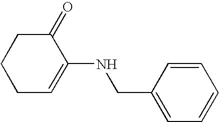

### Preparation of 2-Benzylamino-cyclohex-2-enone

Cyclohex-1,2-dione (1.12 g, 0.01 mol) is added to toluene (10 mL). The mixture is heated to 50° C. under stirring and added benzylamine (0.6 g, 0.005 mol). The mixture is stirred at 80-100° C. for 4 hours. After cooling to room temperature, the solvent is removed in vacuo and the crude is purified by a silica gel column that is eluted with hexane/ethyl acetate to give 2-Benzylamino-cyclohex-2-enone (0.9 g, 79%) with some starting material cyclohex-1,2-dione contaminant.

### Example 11b

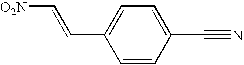

### Preparation of 4-(2-Nitro-vinyl)-benzonitrile

4-Formylbenzonitrile (6.5 g, 0.05 mol), nitromethane (7.56 g, 0.12 mol), and methanol (25 mL) are combined and stirred at 0° C. 1M NaOH (125 mL) is added dropwise to maintain the temperature less than 15° C. Stirring is continued at 0-10° C. for 1 hour. The mixture is added to a solution of 8N HCl (87 mL) slowly to maintain the temperature between 0-10° C. The mixture is stirred at room temperature for 30 min. The solid product is collected and washed with saturated sodium chloride solution. The solid is dissolved in ethyl acetate (100 mL), washed with water (100 mL), and the organic layer is dried over MgSO₄. Solvent is removed and the residue is recrystallized from hexane/ethyl acetate to afford 4-(2-Nitro-vinyl)-benzonitrile as a yellowish solid (5.02 g, 78%).

### Example 11c

### Preparation of 4-(1-Benzyl-7-oxo-4,5,6,7-tetrahydro-1H-indol-3-yl)-benzonitrile

2-Benzylamino-cyclohex-2-enone (0.9 g, 4.5 mmol), 4-(2-nitro-vinyl)benzonitrile (0.78 g, 4.47 mmol), ether (15 mL) and ethyl acetate (10 mL) are combined and stirred at room temperature overnight. The solvent is removed. The residue is extracted with ethyl acetate (100 mL), washed with 1N HCl (2×50 mL) and water (2×50 mL), and the ethyl acetate layer is dried over MgSO₄. The crude product is purified by a silica gel column to afford 4-(1-Benzyl-7-oxo-4,5,6,7-tetrahydro-1H-indol-3-yl)-benzonitrile as a white solid (0.44 g, 29%).

### Example 12

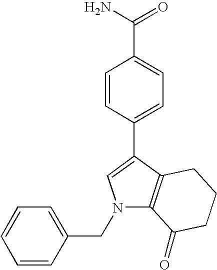

### 4-(1-Benzyl-7-oxo-4,5,6,7-tetrahydro-1H-indol-3-yl)-benzamide

### Compound 11

4-(1-Benzyl-7-oxo-4,5,6,7-tetrahydro-1H-indol-3-yl)benzonitrile (100 mg, 0.3 mmol), 4:1 ethanol/DMSO (5 mL), hydrogen peroxide (30%, 1 mL), and NaOH (1N, 1 mL) are combined and stirred at room temperature for 3 hours. The product is extracted with ethyl acetate (100 mL), washed with water (2×50 mL), and the organic layer is dried over MgSO₄. Solvent is removed and dried in vacuum to afford 4-(1-Benzyl-7-oxo-4,5,6,7-tetrahydro-1H-indol-3-yl)-benzamide as a white solid (105 mg, 99%). LCMS M+H 345.

### Example 13

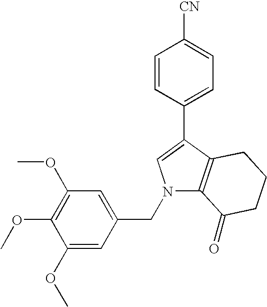

### 4-[7-Oxo-1-(3,4,5-trimethoxy-benzyl)-4,5,6,7-tetrahydro-1H-indol-3-yl]-benzonitrile

### Compound 12

### Example 13a

### Preparation of 7-oxabicyclo[4.1.0]heptan-2-one

To a flask is added cyclohex-2-enone (1.92 g, 0.02 mol), methanol (25 mL), hydrogen peroxide (30%, 6.8 g, 0.06 mol). The mixture is cooled to 0° C. and 6N NaOH (1.67 mL) is added. The reaction is stirred at 0° C. for 45 min and then poured onto ice-water and extracted with dichloromethane (100 mL), washed with brine (2×50 mL), and the organic layer is dried over MgSO₄ and concentrated, affording ca. 0.02 mol of epoxide.

### Example 13b

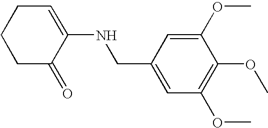

### Preparation of 2-(3,4,5-trimethoxybenzylamino)cyclohex-2-enone

The above cyclohexanone-2,3-epoxide in dichloromethane is dissolved in methanol (20 mL) and added dropwise into a solution of 3,4,5-trimethoxybenzylamine (3.94 g, 0.02 mol) in methanol (10 mL). The mixture is heated at reflux overnight. The solvent is removed in vacuo and the residue is purified by a silica gel column to afford 1.54 g of 2-(3,4,5-trimethoxybenzylamino)cyclohex-2-enone as a white solid (two steps, 26%).

### Example 13c

### Preparation of 4-[7-Oxo-1-(3,4,5-trimethoxy-benzyl)-4,5,6,7-tetrahydro-1H-indol-3-yl]-benzonitrile

2-(N-3,4,5-Trimethoxybenzylamino)-2-cyclohex-2-enone (1.45 g, 5 mmol), 4-(2-nitrovinyl)benzonitrile (0.87 g, 5 mmol), ether (15 mL) and ethyl acetate (10 mL) are combined and stirred at room temperature overnight. The solvent is removed. The residue is extracted with ethyl acetate (100 mL), and washed with 1N HCl (2×50 mL) and brine (2×50 mL). The organic phase is dried over MgSO₄, concentrated, and purified using a silica gel column to afford 4-[7-Oxo-1-(3,4,5-trimethoxy-benzyl)-4,5,6,7-tetrahydro-1H-indol-3-yl]-benzonitrile as a white solid (0.5 g, 24%).

### Example 14

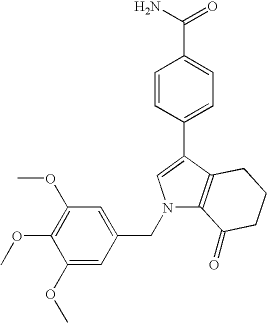

### 4-[7-Oxo-1-(3,4,5-trimethoxy-benzyl)-4,5,6,7-tetrahydro-1H-indol-3-yl]-benzamide

### Compound 13

To a flask is added 4-[7-Oxo-1-(3,4,5-trimethoxy-benzyl)-4,5,6,7-tetrahydro-1H-indol-3-yl]-benzonitrile (100 mg, 0.24 mmol), 4:1 ethanol:DMSO (5 mL), hydrogen peroxide (30%, 1 mL), NaOH (1N, 1 mL). The reaction is stirred at room temperature for 2 hours. The product is extracted with ethyl acetate (100 mL), washed with brine (2×50 mL), dried over MgSO₄. Solvent is removed and dried in vacuum to afford 4-[7-Oxo-1-(3,4,5-trimethoxy-benzyl)-4,5,6,7-tetrahydro-1H-indol-3-yl]-benzamide as a white solid (102 mg, 98%). LCMS M+H 435.

### Example 15

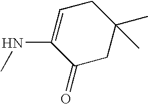

### 5,5-Dimethyl-2-methylamino-cyclohex-2-enone

### Example 15a

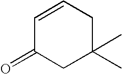

### Preparation of 5,5-Dimethyl-cyclohex-2-enone

To a flask is added 3-chloro-5,5-dimethylcyclohex-2-enone (6.35 g, 0.04 mol), zinc powder (Aldrich 10 micron, 2.61 g, 0.04 mol), potassium iodide (6.64 g, 0.04 mol) and methanol (100 mL). The stirred mixture is heated at reflux for 1 hour. The reaction is cooled to room temperature and the inorganic solids are removed by filtration. The filtrate is concentrated, diluted with ethyl acetate (100 mL), and washed with brine (2×50 mL). The organic phase is dried over MgSO₄, filtered, and concentrated. The crude product is purified by a silica gel column to afford 5,5-Dimethyl-cyclohex-2-enone desired product as an oil (3.15 g, 63%).

### Example 15b

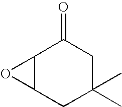

### Preparation of 4,4-Dimethyl-7-oxa-bicyclo[4.1.0]heptan-2-one

To a flask is added 5,5-dimethylcyclo-hex-2-enone (3.15 g, 0.025 mol), methanol (25 mL), hydrogen peroxide (30%, 8.64 g, 0.076 mol). The mixture is cooled to 0° C. and 6N NaOH (2.1 mL) in methanol (10 mL) is added dropwise at 0° C. The reaction is stirred at 0° C. for 45 min and then poured onto ice-water and extracted with dichloromethane (100 mL). The dichloromethane layer is washed with water (2×50 mL), and dried over MgSO₄. The solvent is removed, dried in vacuum to afford 4,4-Dimethyl-7-oxa-bicyclo[4.1.0]heptan-2-one as a colorless oil (2.81 g, 79%).

### Example 15c

### Preparation of 5,5-Dimethyl-2-methylamino-cyclohex-2-enone

5,5-Dimethylcyclohexanone-2,3-epoxide (2.8 g, 0.02 mol) and methylamine (2M in methanol, 40 mL) are combined, heated at reflux for 2 hours and stirred at room temperature overnight. The solvent is removed, the residue is extracted with ethyl acetate (100 mL), washed with brine (2×50 mL), and the organic layer is dried over MgSO₄. The crude product is purified by a silica gel column to afford 5,5-Dimethyl-2-methylamino-cyclohex-2-enone as a colorless oil (2.38, g 78%).

### Example 16

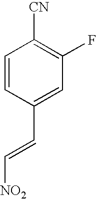

### 2-Fluoro-4-(2-nitro-vinyl)-benzonitrile

### Example 16a

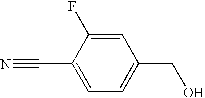

### Preparation of 2-Fluoro-4-hydroxymethyl-benzonitrile

4-cyano-3-fluorobenzoic acid (5 g, 30 mmol) is converted to the corresponding methyl ester using acetone (100 mL), potassium carbonate (7.53 g, 54.5 mmol), and dimethyl sulfate (5.73 g, 45 mmol). The stirred mixture is heated at reflux for 2 hours, cooled, and filtered. The filtrate is concentrated in vacuo, then dissolved in methanol (100 mL). NaBH₄ (2.27 g, 30 mmol) is added, and the reaction stirred at room temperature overnight. The reaction mixture is filtered and the filtrate is concentrated. The residue is diluted with ethyl acetate, washed with brine and the organic layer is dried over MgSO₄. Solvent is removed, affording 2-Fluoro-4-hydroxymethyl-benzonitrile (3.5 g, 78% two steps).

### Example 16b

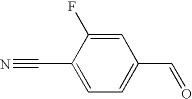

### Preparation of 2-Fluoro-4-formyl-benzonitrile

Pyridinium chlorochromate (7.39 g, 34.27 mmol) is added to a solution of 2-fluoro-4-hydroxymethyl-benzonitrile (5.18 g, 34.27 mmol) in dichloromethane (150 mL), and the mixture is stirred at room temperature for 4 hours. The reaction mixture is passed through a short silica gel pad, eluting with additional dichloromethane. The filtrate is concentrated to afford 2-Fluoro-4-formyl-benzonitrile as a white solid (4.6 g, 90%).

### Example 16c

### Preparation of 2-Fluoro-4-(2-nitro-vinyl)-benzonitrile

2-Fluoro-4-formyl-benzonitrile (2.92 g, 19.6 mmol), nitromethane (2.87 g, 47 mmol) and, methanol (25 mL) are combined and stirred at 0° C. 1M NaOH (47 mL) is added dropwise to maintaining the temperature below 15° C. The reaction is stirred at 0-10° C. for 1 hour, then added to a solution of 8N HCl (34 mL), maintaining the temperature between 0-10° C. The mixture is stirred at room temperature for 30 min. The solid product is collected and washed with water. The solid is dissolved in ethyl acetate (100 mL), washed with brine (50 mL), and the organic layer is dried over MgSO₄. Concentration affords 2-Fluoro-4-(2-nitro-vinyl)-benzonitrile light yellow solid (1.52 g, 40%).

### Example 17

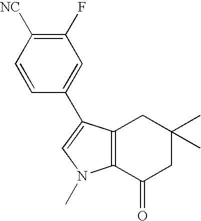

### 2-Fluoro-4-(1,5,5-trimethyl-7-oxo-4,5,6,7-tetrahydro-1H-indol-3-yl)-benzonitrile

### Compound 14

4,4-Dimethyl-7-oxa-bicyclo[4.1.0]heptan-2-one (1.13 g, 7.39 mmol), 2-fluoro-4-(2-nitro-vinyl)benzonitrile (1.42 g, 7.39 mmol), ether (15 mL) and ethyl acetate (10 mL) are stirred at room temperature overnight. The reaction is concentrated and diluted with ethyl acetate (100 mL). The solution is washed with 1N HCl (2×50 mL) and brine (2×50 mL). The organic layer is separated and dried over MgSO₄. The crude product is purified by silica gel chromatography to afford 2-Fluoro-4-(1,5,5-trimethyl-7-oxo-4,5,6,7-tetrahydro-1H-indol-3-yl)-benzonitrile (0.7 g 32%).

### Example 18

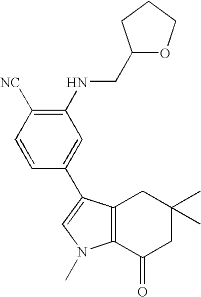

### 2-[(Tetrahydro-furan-2-ylmethyl)-amino]-4-(1,5,5-trimethyl-7-oxo-4,5,6,7-tetrahydro-1H-indol-3-yl)-benzonitrile

### Compound 15

To a microwave reactor vessel is added 2-fluoro-4-(1,5,5-trimethyl-7-oxo-4,5,6,7-tetrahydro-1H-indol-3-yl)benzonitrile (0.2 g, 0.67 mmol), tetrahydrofuran-2-ylmethylamine (0.14 g, 1.35 mmol), N,N-diisopropylethylamine (0.17 g, 1.35 mmol), DMSO (2 mL). The mixture is microwaved at 170° C. for 2000 sec (low absorbance). The reaction is extracted with ethyl acetate (100 mL) and washed with brine (2×50 mL). The organic layer is dried over MgSO₄. The crude product is purified by a silica gel column to afford 2-[(Tetrahydro-furan-2-ylmethyl)-amino]-4-(1,5,5-trimethyl-7-oxo-4,5,6,7-tetrahydro-1H-indol-3-yl)-benzonitrile (0.21 g, 84%).

### Example 19

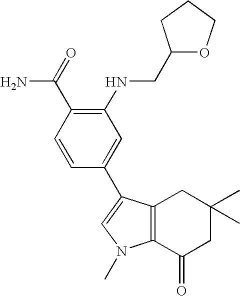

### 2-[(Tetrahydro-furan-2-ylmethyl)-amino]-4-(1,5,5-trimethyl-7-oxo-4,5,6,7-tetrahydro-1H-indol-3-yl-benzamide

### Compound 16

To a flask is added 2-[(Tetrahydro-furan-2-ylmethyl)-amino]-4-(1,5,5-trimethyl-7-oxo-4,5,6,7-tetrahydro-1H-indol-3-yl)-benzonitrile (0.21 g, 5.56 mmol), 10 ml of ethanol/DMSO (4:1), hydrogen peroxide (30%, 1 mL), NaOH (1N, 1 mL). The reaction is stirred at room temperature for 3 hours. The product is extracted with ethyl acetate (100 mL), washed with brine (2×50 mL), and the organic layer is dried over MgSO₄. Solvent is removed and the crude is purified by a silica gel column to afford 2-[(Tetrahydro-furan-2-ylmethyl)-amino]-4-(1,5,5-trimethyl-7-oxo-4,5,6,7-tetrahydro-1H-indol-3-yl)-benzamide (100 mg, 45%). LCMS M+H 396.

### Example 20

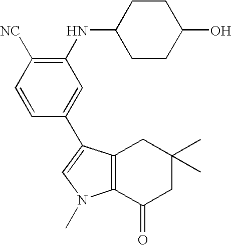

### 2-(4-Hydroxy-cyclohexylamino)-4-(1,5,5-trimethyl-7-oxo-4,5,6,7-tetrahydro-1H-indol-3-yl)-benzonitrile

### Compound 17

To a microwave reactor vessel is added 2-fluoro-4-(1,5,5-trimethyl-7-oxo-4,5,6,7-tetrahydro-1H-indol-3-yl)benzonitrile (0.2 g, 0.67 mmol), trans-4-hydroxycyclohexylamine (0.156 g, 1.35 mmol), N,N-diisopropylethylamine (0.17 g, 1.35 mmol), DMSO (2 mL). The mixture is microwaved at 170° C. for 2000 sec at low absorbance. The reaction is extracted with ethyl acetate (100 mL) and washed with brine (2×50 mL). The organic layer is dried over MgSO₄. The crude product is purified by a silica gel column to afford 2-(4-Hydroxy-cyclohexylamino)-4-(1,5,5-trimethyl-7-oxo-4,5,6,7-tetrahydro-1H-indol-3-yl)-benzonitrile as a white solid (0.26 g, 100%).

### Example 21

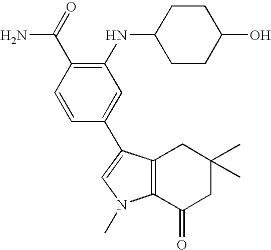

### 2-(4-Hydroxy-cyclohexylamino)-4-(1,5,5-trimethyl-7-oxo-4,5,6,7-tetrahydro-1H-indol-3-yl)-benzamide

### Compound 18

2-(4-Hydroxy-cyclohexylamino)-4-(1,5,5-trimethyl-7-oxo-4,5,6,7-tetrahydro-1H-indol-3-yl)-benzonitrile (0.26 g, 6.6 mmol), 10 ml of ethanol/DMSO (4:1), and hydrogen peroxide (30%, 1 mL), NaOH (1N, 1 mL) are stirred at room temperature for 3 hours. The product is extracted with ethyl acetate (100 mL), washed with brine (2×50 mL), and dried over MgSO₄. Solvent is removed and the crude is purified by a silica gel column to afford 2-(4-Hydroxy-cyclohexylamino)-4-(1,5,5-trimethyl-7-oxo-4,5,6,7-tetrahydro-1H-indol-3-yl)-benzamide as a white solid (100 mg, 37%). LLCMS M+H 410.

### Example 22

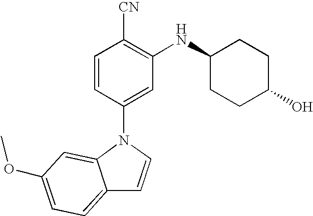

### 2-(4-Hydroxycyclohexylamino)-4-(6-methoxyindol-1-yl)benzonitrile

### Compound 19

### Example 22a

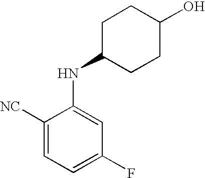

### Preparation of 4-fluoro-2-(4-hydroxycyclohexylamino)benzonitrile

2,4-Difluorobenzonitrile (50.0 g, 0.359 mol), trans-4-aminocyclohexanol (41.4 g, 0.359 mol, 1 equiv.), and N,N-diisopropylethylamine (62.6 mL, 0.359 mol, 1 equiv.) are dissolved in 300 mL of DMSO. The reaction vessel is outfitted with a reflux condenser to avoid loss of N,N-diisopropylethylamine. The reaction is then placed in an oil bath that had been pre-heated to 150° C., and is stirred at this temperature for 20 minutes. The solution is then cooled, poured into 750 mL of saturated aqueous NH₄Cl, and extracted with ethyl acetate (200 mL×3). The combined organics are washed with brine (150 mL×3), dried over Na₂SO₄, filtered, and concentrated in vacuo. The resultant residue is purified by column chromatography (1:1 ethyl acetate/hexane) to afford 20.9 g (25% yield) of the desired 4-fluoro-2-(4-hydroxycyclohexylamino)benzonitrile as a white powder, and 36.1 g (43% yield) of the undesired isomer as a white powder.

### Example 22b

### Preparation of 2-(4-hydroxycyclohexylamino)-4-(6-methoxyindol-1-yl)benzonitrile

To a 20 mL vial with a stirbar are added 6-methoxyindole (50 mg, 0.34 mmol) and 4-fluoro-2-(4-hydroxycyclohexylamino)benzonitrile (95 mg, 0.41 mmol). The reagents are flushed with N₂ and dissolved in anhydrous DMF (1.9 mL). NaH (17 mg, 0.68 mmol) is added and the reaction is heated to 155° C. for 16 hours. The reaction is cooled to room temperature and diluted with EtOAc (50 mL). The organic layer is washed with H₂O (3×50 mL), dried over Na₂SO₄, and concentrated. The mixture is purified by gradient flash chromatography eluting with 0% to 100% EtOAc in hexanes to provide 2-(4-hydroxycyclohexylamino)-4-(6-methoxyindol-1-yl)benzonitrile as a yellow oil (40 mg, 33% yield) (LC/MS M+H 362).

### Example 23

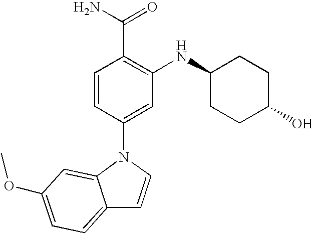

### 2-(4-Hydroxycyclohexylamino)-4-(6-methoxyindol-1-yl)benzamide

### Compound 20

Ethanol (0.35 mL) and DMSO (0.09 mL) are added to 2-(4-hydroxycyclohexylamino)-4-(6-methoxyindol-1-yl)benzonitrile (40 mg, 0.111 mmol). To the solution is added 1 M aqueous NaOH (0.01 mL) and 30% aqueous H₂O₂ (0.01 mL) dropwise. The reaction is stirred at room temperature for 1 hour and diluted with EtOAc (10 mL). The organic layer is washed with brine (5 mL), dried over Na₂SO₄, and concentrated. Purification by gradient flash chromatography eluting with 0% to 100% EtOAc in hexanes affords 2-(4-hydroxycyclohexylamino)-4-(6-methoxyindol-1-yl)benzamide as a white solid (24 mg, 57% yield) (LC/MS M+H=380).

### Example 24

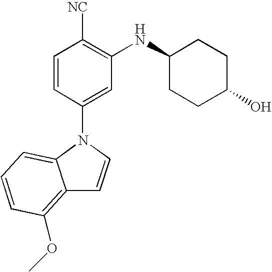

### 2-(4-Hydroxycyclohexylamino)-4-(4-methoxyindol-1-yl)benzonitrile

### Compound 21

To a 20 mL vial with a stirbar are added 4-methoxyindole (150 mg, 1.02 mmol) and 4-fluoro-2-(4-hydroxycyclohexylamino)benzonitrile (287 mg, 1.22 mmol). The reagents are flushed with N₂ and dissolved in anhydrous DMF (5.7 mL). NaH (51 mg, 2.04 mmol) is added and the reaction is heated to 155° C. for 16 hours. The reaction is cooled to room temperature and diluted with EtOAc (100 mL). The organic layer is washed with H₂O (3×100 mL), dried over Na₂SO₄, and concentrated. The mixture is purified by gradient flash chromatography eluting with 0% to 100% EtOAc in hexanes to provide 2-(4-hydroxycyclohexylamino)-4-(4-methoxyindol-1-yl)benzonitrile as a yellow oil (125 mg, 34% yield) (LC/MS M+H 362).

### Example 25

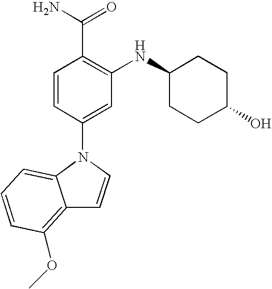

### 2-(4-Hydroxycyclohexylamino)-4-(4-methoxyindol-1-yl)benzamide

### Compound 22

EtOH (0.84 mL) and DMSO (0.21 mL) are added to 2-(4-hydroxycyclohexylamino)-4-(4-methoxyindol-1-yl)benzonitrile (95 mg, 0.263 mmol). To the solution is added 1 M aqueous NaOH (0.05 mL) and 30% aqueous H₂O₂ (0.05 mL) dropwise. The reaction is stirred at room temperature for 1 hour and diluted with EtOAc (50 mL). The organic layer is washed with brine (50 mL), dried over Na₂SO₄, and concentrated. Purification by gradient flash chromatography eluting with 0% to 100% EtOAc in hexanes affords 2-(4-hydroxycyclohexylamino)-4-(4-methoxyindol-1-yl)benzamide as a white solid (73 mg, 73% yield) (LC/MS M+H=380).

### Example 26

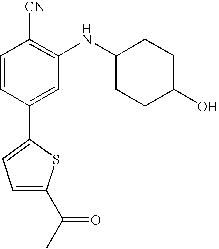

### 4-(5-Acetyl-thiophen-2-yl)-2-(4-hydroxy-cyclohexylamino)-benzonitrile

### Compound 23

### Example 26a

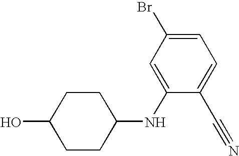

### Preparation of 4-Bromo-2-(4-hydroxy-cyclohexylamino)-benzonitrile

4-Bromo-2-fluorobenzonitrile (4 mmol, 800 mg), trans-4-aminocyclohexanol (6 mmol, 690 mg), diisopropylethylamine (5 mmol, 0.87 mL), and dimethylsulfoxide (4 mL) are sealed in a Personal Chemistry Microwave tube, and the reaction is irradiated at 150 degrees Celsius on high absorbance for 900 seconds. The product is taken up in ethyl acetate (150 mL) and washed with water (100 mL). The organic layer is dried over magnesium sulfate, concentrated, and the residue subjected to chromatography, affording the desired 4-Bromo-2-(4-hydroxy-cyclohexylamino)-benzonitrile as a white solid (1.01 g, 85%).

### Example 26b

### Preparation of 4-(5-Acetyl-thiophen-2-yl)-2-(4-hydroxy-cyclohexylamino)-benzonitrile

4-Bromo-2-(4-hydroxy-cyclohexylamino)-benzonitrile (0.5 mmol, 147 mg) is combined with 5-acetyl-2-thiopheneboronic acid (0.75 mmol, 128 mg), sodium carbonate (0.75 mmol, 80 mg) and bis-(triphenylphosphine)palladiumdichloride (0.01 mmol, 7 mg) in a Personal Chemistry Microwave tube. After dilution with 2 mL of 7:3:2 dimethoxyethane:ethanol:water, the tube is sealed and irradiated at 140 degrees Celsius at high absorbance for 800 seconds. The mixture is diluted with ethyl acetate (75 mL) and washed with water (50 mL). The organic phase is dried using magnesium sulfate, concentrated and chromatographed, affording 4-(5-Acetyl-thiophen-2-yl)-2-(4-hydroxy-cyclohexylamino)-benzonitrile as a yellow foam (102 mg, 60%). LCMS m/z M+=340.

### Example 27

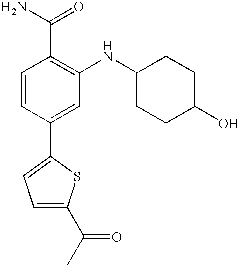

### 4-(5-Acetyl-thiophen-2-yl)-2-(4-hydroxy-cyclohexylamino)-benzamide

### Compound 24

The 4-(5-Acetyl-thiophen-2-yl)-2-(4-hydroxy-cyclohexylamino)-benzonitrile (0.30 mmol, 96 mg) is diluted with dimethylsulfoxide (8 drops) and ethanol (2 mL). KOH (89 mg) is added, and the mixture is warmed in a 45 degree Celsius oil bath. 30% hydrogen peroxide (˜0.5 mL) is added. After 30 minutes, extraction with ethyl acetate (100 mL)/water (50 mL), followed by treatment of the organic layer with magnesium sulfate, affords the desired thienylbenzamide, which is purified by trituration with ethyl acetate, yielding 4-(5-Acetyl-thiophen-2-yl)-2-(4-hydroxy-cyclohexylamino)-benzamide as a yellow solid (83 mg, 77%). LC/MS m/z=359 [M+H]⁺.

### Example 28

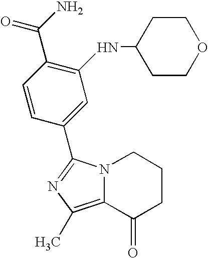

### 4-(1-methyl-8-oxo-5,6,7,8-tetrahydroimidazo[1,5-a]pyridin-3-yl)-2-(tetrahydro-2H-pyran-4-ylamino)benzamide

### Synthesis of 5-methyl-1-trityl-1H-imidazole-4-carbaldehyde

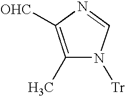

Triphenylmethyl chloride (12.70 g, 45.56 mmol) is added to a solution of 5-methyl-1H-imidazole-4-carbaldehyde (5.00 g, 45.41 mmol) and triethylamine (12.60 mL, 9.15 g, 90.40 mmol) in DMF (100 mL). The mixture is stirred at room temperature for 24 h, at which time additional triphenylmethyl chloride (5.00 g, 17.94 mmol) and triethylamine (2.00 mL, 1.45 g, 14.35 mmol) are added. After stirring for another 18 h, water (100 mL) and ethyl acetate (150 mL) are added. The layers are separated, and the aqueous layer is extracted with ethyl acetate (3×100 mL). The combined organic layers are washed with brine (5×75 mL), dried over Na₂SO₄, filtered, and concentrated at reduced pressure. Chromatography (silica gel flash column, 85:15 to 60:40 hexanes/ethyl acetate) affords a mixture of N-trityl protected 5-methyl-1-trityl-1H-imidazole-4-carbaldehyde (12.00 g, 77%) as a white solid: ESI MS m/z 353 [M+H]⁺.

### Synthesis of 1-(5-methyl-1-trityl-1H-imidazol-4-yl)-4-(tetrahydro-2H-pyran-2-yloxy)but-2-yn-1-ol

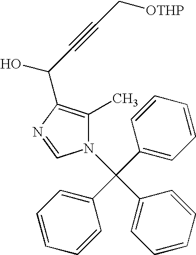

A 1.0 M n-butyl lithium solution in hexanes (70 mL, 70 mmol) is added dropwise to a solution of tetrahydro-2-(2-propynyloxy)-2H-pyran (8.82 mL, 8.79 g, 62.73 mmol) in THF (250 mL) cooled to −78° C. The resulting solution is stirred at −78° C. for 5 min, and then allowed to warm to room temperature. After and stirring for 20 min, this solution is transferred via cannula over 30 min to a solution of 5-methyl-1-trityl-1H-imidazole-4-carbaldehyde (14.71 g, 41.74 mmol) in THF (250 mL) cooled to −78° C. The resulting mixture is maintained at −78° C. for 1 h, and then allowed to warm to room temperature. After 45 min, water (200 mL) and ethyl acetate (200 mL) are added. The layers are separated, and the aqueous layer is extracted with ethyl acetate (2×100 mL). The organic layers are combined, washed with brine, dried over Na₂SO₄, filtered and concentrated at reduced pressure. Chromatography of the residue (silica gel flash column, 85:15 to 40:60 hexanes/ethyl acetate to elute residual starting material and 99.5:0.5 to 92:8 dichloromethane/methanol to elute the product) affords 1-(5-methyl-1-trityl-1H-imidazol-4-yl)-4-(tetrahydro-2H-pyran-2-yloxy)but-2-yn-1-ol (19.23 g, 93%) as an oil: APCI MS m/z 493 [M+H]⁺.

### Synthesis of 1-(5-methyl-1-trityl-1H-imidazol-4-yl)-4-(tetrahydro-2H-pyran-2-yloxy)but-2-yn-1-one

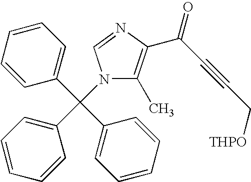

Manganese (IV) oxide (50 g, 87 mmol) is added to a solution of 1-(5-methyl-1-trityl-1H-imidazol-4-yl)-4-(tetrahydro-2H-pyran-2-yloxy)but-2-yn-1-ol (19.23 g, 39.04 mmol) in dichloromethane (900 mL). The suspension is stirred at room temperature overnight, and then filtered through Celite. The filter cake is rinsed with dichloromethane (3×100 mL), and the filtrate is concentrated at reduced pressure. Chromatography (silica gel flash column, 67:33 to 50:50 hexanes/ethyl acetate) yields 1-(5-methyl-1-trityl-1H-imidazol-4-yl)-4-(tetrahydro-2H-pyran-2-yloxy)but-2-yn-1-one (10.97 g, 57%) as an oil: ¹H NMR (CDCl₃, 500 MHz) δ 7.36-7.33 (m, 10H), 7.14-7.11 (m, 6H), 4.91 (t, J=3.4 Hz, 1H), 4.52 (s, 2H), 3.85 (ddd, J=11.5, 9.5, 2.9 Hz, 1H), 3.54 (dtd, J=11.5, 4.4, 1.3 Hz, 1H), 1.86 (s, 3H), 1.85-1.72 (m, 2H), 1.62-1.54 (m, 4H).

### Synthesis of 1-(5-methyl-1-trityl-1H-imidazol-4-yl)-4-(tetrahydro-2H-pyran-2-yloxy)butan-1-one5

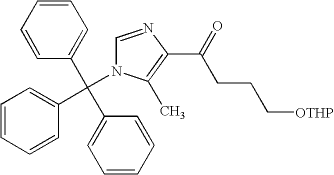

Palladium on carbon (10%, wet, 0.4 g) is added to a solution of 1-(5-methyl-1-trityl-1H-imidazol-4-yl)-4-(tetrahydro-2H-pyran-2-yloxy)but-2-yn-1-one (2.74 g, 5.58 mmol) in THF (250 mL) and ethyl acetate (80 mL). The stirred suspension is degassed with three pump/nitrogen backfill cycles, and then charged with 1 atm of hydrogen gas. After stirring for 2 h, the hydrogen is removed under vacuum and the flask backfilled with nitrogen. The catalyst is removed by filtration through a pad of Celite. The filter cake is rinsed with ethyl acetate (3×30 mL), and the filtrate is concentrated at reduced pressure. The crude residue obtained is combined with that from three more reactions of the same scale and chromatographed (silica gel flash column, 90:10 to 67:33 hexanes/ethyl acetate) to afford 1-(5-methyl-1-trityl-1H-imidazol-4-yl)-4-(tetrahydro-2H-pyran-2-yloxy)butan-1-one (9.71 g, 88%) as a white foam: ¹H NMR (CDCl₃, 300 MHz) δ 7.30-7.25 (m, 9H), 7.20 (s, 1H), 7.08-7.04 (m, 6H), 4.54 (t, J=2.9 Hz, 1H), 3.83-3.72 (m, 2H), 3.45-3.37 (m, 2H), 1.99-1.90 (m, 2H), 1.78 (s, 3H), 1.56-1.39 (m, 6H).

### Synthesis of 4-hydroxy-1-(5-methyl-1-trityl-1H-imidazol-4-yl)butan-1-one

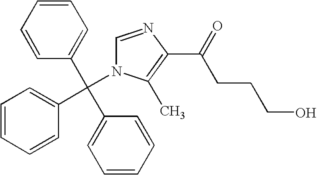

A 3 N HCl solution (36 mL) is added dropwise over 10 min to a solution of 1-(5-methyl-1-trityl-1H-imidazol-4-yl)-4-(tetrahydro-2H-pyran-2-yloxy)butan-1-one (9.71 g, 19.63 mmol) in THF (630 mL). After stirring for 3.5 h at room temperature, sat. NaHCO₃ (40 mL) and solid NaHCO₃ are added followed by ethyl acetate (200 mL) and water (200 mL). The layers are separated, and the aqueous layer is extracted with ethyl acetate (3×100 mL). The organic layers are combined, washed with brine, dried over Na₂SO₄, filtered, and concentrated at reduced pressure. Chromatography (silica gel flash column, 80:20 to 20:80 hexanes/ethyl acetate) gives 4-hydroxy-1-(5-methyl-1-trityl-1H-imidazol-4-yl)butan-1-one (5.5 g, 68%) as an oil: ¹H NMR (CDCl₃, 500 MHz) δ 7.36-7.33 (m, 9H), 7.29 (s, 1H), 7.14-7.11 (m, 6H), 3.77 (br s, 1H), 3.65 (t, J=5.6 Hz, 2H), 3.13 (t, J=6.6 Hz, 2H), 1.99-1.96 (m, 2H), 1.85 (s, 3H).

### Synthesis of 1-methyl-6,7-dihydroimidazo[1,5-a]pyridin-8(5H)-one

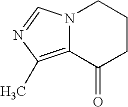

Methanesulfonyl chloride (1.76 mL, 2.59 g, 22.65 mmol) is added dropwise at room temperature to a stirred solution of 4-hydroxy-1-(5-methyl-1-trityl-1H-imidazol-4-yl)butan-1-one (3.81 g, 9.28 mmol) and triethylamine (3.07 mL, 2.23 g, 22.03 mmol) in THF (150 mL). After stirring at room temperature for 1.5 h, the white suspension is cooled to 0° C., and water (6.8 mL) is added dropwise. The resulting mixture is extracted with ethyl acetate (2×50 mL), and the combined organic layers are washed with brine, dried over Na₂SO₄, filtered, and concentrated at reduced pressure. The residue obtained is redissolved in acetonitrile (100 mL) and heated at reflux for 1 h. The solution is allowed to cool to room temperature, and the resulting precipitate is collected by filtration to afford 1-methyl-6,7-dihydroimidazo[1,5-a]pyridin-8(5H)-one (0.665 g, 48%) as a white solid: ESI MS m/z 151 [M+H]⁺.

### Synthesis of 3-bromo-1-methyl-6,7-dihydroimidazo[1,5-a]pyridin-8(5H)-one

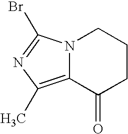

N-Bromosuccinimide (1.19 g, 6.69 mmol) is added to a suspension of 1-methyl-6,7-dihydroimidazo[1,5-a]pyridin-8(5H)-one (0.668 g, 4.45 mmol) in acetonitrile (55 mL). The mixture is stirred at room temperature, protected from light. After 1 h, a second portion of N-bromosuccinimide (0.2 g, 1.12 mmol) is added. After an additional 2 h, the solvent is removed at reduced pressure, and the residue obtained is adsorbed onto silica gel and chromatographed (silica gel flash column, dichloromethane to 85:15 dichloromethane/methanol) to afford 3-bromo-1-methyl-6,7-dihydroimidazo[1,5-a]pyridin-8(5H)-one that was still impure. Rechromatography (silica gel preparative TLC, 50:50 hexanes/ethyl acetate) yields clean 3-bromo-1-methyl-6,7-dihydroimidazo[1,5-a]pyridin-8(5H)-one (0.072 g, 7%) as a white solid: ESI MS m/z 229 [M+H]⁺.

### Synthesis of 2-fluoro-4-(1-methyl-8-oxo-5,6,7,8-tetrahydroimidazo[1,5-a]pyridin-3-yl)benzonitrile

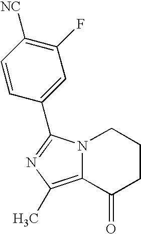

A mixture of 3-bromo-1-methyl-6,7-dihydroimidazo[1,5-a]pyridin-8(5H)-one (0.072 g, 0.314 mmol), 4-cyano-3-fluorophenylboronic acid (0.06 g, 0.364 mmol), 2 M K₂CO₃, toluene (5 mL) and methanol (1 mL) is degassed with three pump/argon backfill cycles.

Tetrakis(triphenylphosphine)palladium (0) (0.036 g, 0.0312 mmol) is added, and the degas cycle is repeated two times. The mixture is then heated at reflux for 18 h. After cooling to room temperature, the solvent is removed at reduced pressure, and the residue obtained is chromatographed (silica gel preparative TLC, 50:50 hexanes/ethyl acetate) to afford 2-fluoro-4-(1-methyl-8-oxo-5,6,7,8-tetrahydroimidazo[1,5-a]pyridin-3-yl)benzonitrile, as a mixture with triphenylphosphine oxide (0.053 g): ESI MS m/z 270 [M+H]⁺. It was used in the next step without further purification.

### Synthesis of 4-(1-methyl-8-oxo-5,6,7,8-tetrahydroimidazo[1,5-a]pyridin-3-yl)-2-(tetrahydro-2H-pyran-4-ylamino)benzonitrile

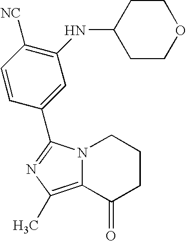

A mixture of -fluoro-4-(1-methyl-8-oxo-5,6,7,8-tetrahydroimidazo[1,5-a]pyridin-3-yl)benzonitrile (est. 0.403 g, 0.4 mmol), 4-aminotetrahydropyran (0.02 g, 0.198 mmol), and N,N-diisopropylethylamine (0.04 mL, 0.03 g, 0.23 mmol) in DMSO (2 mL) is heated at 100° C. for 24 h. After that time, the mixture is cooled to room temperature and diluted with water (5 mL) and ethyl acetate (5 mL). The layers are separated, and the aqueous layer is extracted with ethyl acetate (3×10 mL). The combined organic layers are washed with water (4×5 mL) and brine (4×5 mL), dried over Na₂SO₄, filtered, and concentrated at reduced pressure. The residue obtained is chromatographed (silica gel preparative TLC, 75:25 ethyl acetate/hexanes) to give 4-(1-methyl-8-oxo-5,6,7,8-tetrahydroimidazo[1,5-a]pyridin-3-yl)-2-(tetrahydro-2H-pyran-4-ylamino)benzonitrile (0.027 g, 19% over two steps) as a light yellow solid: ESI MS m/z 351 [M+H]⁺.

### Synthesis of Title Compound

A 2 N NaOH solution (1 drop) and 3% hydrogen peroxide (2 drops) are added to a stirred suspension of 4-(1-methyl-8-oxo-5,6,7,8-tetrahydroimidazo[1,5-a]pyridin-3-yl)-2-(tetrahydro-2H-pyran-4-ylamino)benzonitrile (0.022 g, 0.063 mmol) in ethanol (2 mL) and DMSO (0.5 mL). After the mixture is stirred at room temperature for 5 min, water (5 mL) and ethyl acetate (5 mL) are added. The aqueous layer is separated and extracted with ethyl acetate (3×15 mL). The organic layers are combined, washed with water (3×10 mL) and brine (3×10 mL). The combined aqueous layer is back-extracted with ethyl acetate (10 mL), and the combined organic layers are dried over Na₂SO₄, filtered and concentrated at reduced pressure. The residue obtained is chromatographed (silica gel preparative TLC, 97:3 dichloromethane/methanol) to afford 4-(1-methyl-8-oxo-5,6,7,8-tetrahydroimidazo[1,5-a]pyridin-3-yl)-2-(tetrahydro-2H-pyran-4-ylamino)benzamide (0.019 g, 83%) as a yellow solid: ESI MS m/z 369 [M+H]⁺.

### Example 29

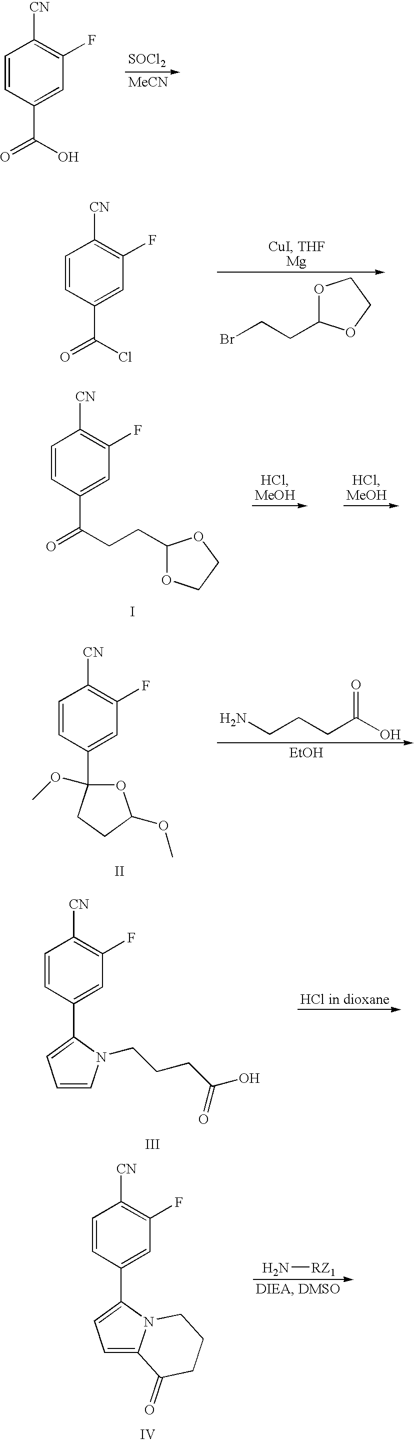

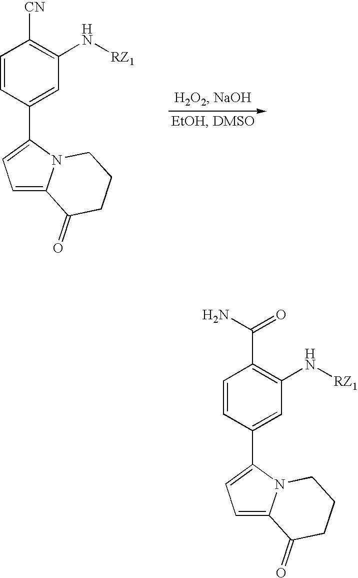

### Synthesis of 4-cyano-3-fluorobenzoyl chloride

A solution of 4-cyano-3-fluorobenzoic acid (5 g, 30 mmol) and SOCl₂ (10 mL) in acetonitrile (20 mL) is heated to reflux for 2 h. After cooling to room temperature, the solvent is removed under reduced pressure. The residue is dried in vacuo to afford 4 g of 4-cyano-3-fluorobenzoyl chloride.

### Synthesis of Compound I

A three-neck flask is charged with Mg (1.27 g) and dry THF (20 mL). Under constant stirring, 2-(2-bromoethyl)1,3-dioxalane (4.73 g, 26.15 mmol) is added slowly, keeping the temperature between 25-30° C. After the addition is complete, the reaction is stirred at room temperature for 30 min. The solution is transferred into a new flask to remove the excess Mg. The solution is then cooled to 0° C., and CuI (4.98 g, 26.15 mmol) is added portionwise. The reaction mixture is stirred at 0° C. for 20 min, then cooled at −75° C. A solution of 4-cyano-3-fluorobenzoyl chloride (4 g, 21.8 mmol) in THF is added dropwise, and the reaction mixture is stirred at −75° C. for 1 h, then at RT overnight. The reaction mixture is poured into ice, and 2 N HCl (100 mL). The aqueous layer is extracted with EtOAc. The combined organic layers are washed with brine, dried over MgSO₄, and evaporated under reduced pressure. The residue is dried in vacuo to afford 5.7 g of crude 1,4-(3-(1,3-dioxolan-2-yl)propanoyl)-2-fluorobenzonitrile. LC/MS m/z=250 [M+H]⁺.

### Synthesis of Compound II

Crude 4-(3-(1,3-dioxolan-2-yl)propanoyl)-2-fluorobenzonitrile (I) (5.7 g, crude) is dissolved in MeOH (150 mL), and 2 N HCl (80 mL). The reaction mixture is stirred overnight at RT. The aqueous layer is extracted with EtOAc. The combined organic layers are washed with brine, dried over MgSO₄, and evaporated under reduced pressure. The residue is dried in vacuo to afford 5 g of crude II, 4-(2,5-dimethoxytetrahydrofuran-2-yl)-2-fluorobenzonitrile. LC/MS m/z=252 [M+H]⁺. The product is used without further purification.

### Synthesis of Compound III

To a solution of 4-(2,5-dimethoxytetrahydrofuran-2-yl)-2-fluorobenzonitrile (II) (5 g, 20 mmol) in EtOH (100 mL) is added 4-aminobutyric acid (4.12 g, 40 mmol). The reaction mixture is heated to reflux for 2.5 days. The reaction mixture is then cooled to RT. After evaporation of the solvent under reduced pressure, the residue is extracted with EtOAc. The combined organic layers are washed with brine, dried over MgSO₄, and evaporated under reduced pressure. The residue is dried in vacuo to afford 0.7 g of crude product III, 4-(2-(4-cyano-3-fluorophenyl)-1H-pyrrol-1-yl)butanoic acid. LC/MS m/z=273 [M+H]⁺.

### Synthesis of Compound IV

Crude 4-(2-(4-cyano-3-fluorophenyl)-1H-pyrrol-1-yl)butanoic acid (III) (0.7 g, 2.57 mmol) is dissolved in 4M HCl in dioxane (20 mL). The reaction mixture is stirred at RT for 3 h. The aqueous layer is extracted with EtOAc. The combined organic layers are washed with 1N NaOH, dried over MgSO₄, and evaporated under reduced pressure. The residue is purified using a silica gel column to afford 0.06 g of IV, 2-fluoro-4-(8-oxo-5,6,7,8-tetrahydroindolizin-3-yl)benzonitrile. LC/MS m/z=255 [M+H]⁺.

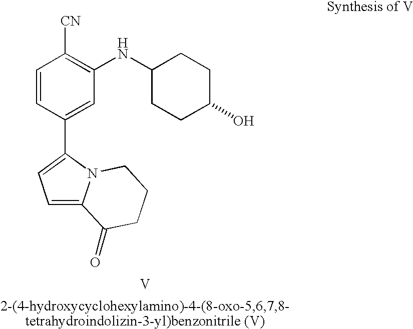

A solution of 2-fluoro-4-(8-oxo-5,6,7,8-tetrahydroindolizin-3-yl)benzonitrile (IV) (0.06 g, 0.24 mmol), 4-trans-aminocyclohexanol (0.055 g, 0.48 mmol) and diisopropylethylamine (0.062 g, 0.48 mmol) in DMSO (2 mL) is microwaved at 150° C. for 1500 s. The reaction mixture is extracted with EtOAc. The combined organic layers are washed with water, dried over MgSO₄, and evaporated under reduced pressure. The residue is purified using a silica gel column to afford 0.07 g of V, 2-(4-hydroxycyclohexylamino)-4-(8-oxo-5,6,7,8-tetrahydroindolizin-3-yl)benzonitrile. LC/MS m/z=350 [M+H]⁺.

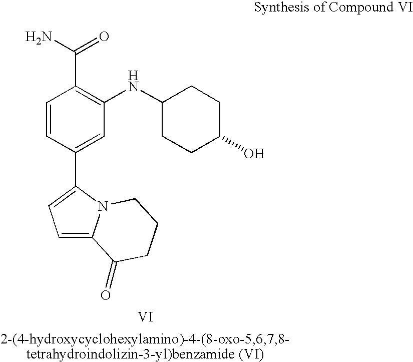

A solution of 2-(−4-hydroxycyclohexylamino)-4-(8-oxo-5,6,7,8-tetrahydroindolizin-3-yl)benzonitrile (V) (0.07 g, 0.2 mmol) in EtOH/DMSO (4:1, 10 mL) is treated with 30% H₂O₂ (0.5 mL), and 1 N NaOH (0.5 mL). The reaction mixture is stirred at RT for 3 h. The reaction mixture is poured into water. The aqueous layer is extracted with EtOAc. The combined organic layers are washed with brine, dried over MgSO₄, and evaporated under reduced pressure. The residue is dried in vacuo to afford 0.05 g of crude product VI, 2-(4-hydroxycyclohexylamino)-4-(8-oxo-5,6,7,8-tetrahydroindolizin-3-yl)benzamide. LC/MS m/z=368 [M+H]⁺.

### Example 30

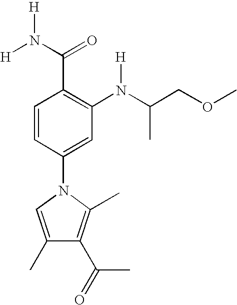

### 4-(3-acetyl-2,4-dimethyl-1H-pyrrol-1-yl)-2-(1-methoxypropan-2-ylamino)benzamide

### Part A

### Preparation of 4-(3-Acetyl-2,4-dimethyl-pyrrol-1-yl)-2-bromo-benzonitrile

60% Sodium Hydride in oil (0.40 g, 10 mmol) is triturated with hexane. N,N-dimethylformamide (4 mL) is added and the flask is chilled to 0 degrees Celsius. 3-acetyl-2,4-dimethylpyrrole (685 mg, 5 mmol) is added. After 5 min, 2-bromo-4-fluorobenzonitrile (1.2 g, 6 mmol) is added. The reaction is stirred for 1 h at 60 degrees Celsius, then cooled and taken up in ethyl acetate (200 ml)/water (100 mL). The organic layer is dried over magnesium sulfate, filtered, concentrated, and subjected to chromatography, to afford 4-(3-Acetyl-2,4-dimethyl-pyrrol-1-yl)-2-bromo-benzonitrile as a tan solid 741 mg (47%). LC/MC m/z=317 [M+H]⁺.

### Part B

### Preparation of 4-(3-Acetyl-2,4-dimethyl-pyrrol-1-yl)-2-(2-methoxy-1-methyl-ethylamino)-benzamide

The bromonitrile (317 mg, 1 mmol) from part A is combined with 2-amino-1-methoxypropane (0.21 mL, 2 mmol), sodium t-butoxide (192 mg, 2 mmol), toluene (2 mL), DPPF (58 mg), and palladium (II) acetate (30 mg) in a sealed microwave reactor and irradiated at 110 degrees Celsius for 900 sec at high absorbance. The mixture is extracted with ethyl acetate (150 mL) and washed with water (50 mL). The organic layer is dried over magnesium sulfate, filtered through a silica plug and concentrated, affording 4-(3-Acetyl-2,4-dimethyl-pyrrol-1-yl)-2-(2-methoxy-1-methyl-ethylamino)-benzonitrile. LC/MC m/z=326 [M+H]⁺.

The crude 4-(3-Acetyl-2,4-dimethyl-pyrrol-1-yl)-2-(2-methoxy-1-methyl-ethylamino)-benzonitrile is hydrolyzed using DMSO (7 drops), KOH (165 mg, ˜3 mmol), ethanol (3 mL) and 30% hydrogen peroxide (˜3 mL) for 75 min at ambient temperature. The reaction is taken up in ethyl acetate (150 mL)/water (50 mL). The organic layer is dried, concentrated, and subjected to chromatography, affording the expected 4-(3-Acetyl-2,4-dimethyl-pyrrol-1-yl)-2-(2-methoxy-1-methyl-ethylamino)-benzamide as a white foam (162 mg, 47% from the bromonitrile of part A). LC/MC m/z=344 [M+H]⁺.

### Example 31

The following compounds are prepared essentially according to the procedures set forth in the above schemes and detailed in the preceding examples.

| Compound | | | |
|---|---|---|---|
| No. | Structure | Name | [M + H]⁺ |
| 31 | 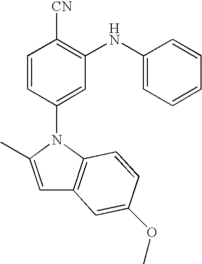 | 4-(5-methoxy-2-methyl-1H-indol-1-yl)-2-(phenylamino)benzonitrile | 354 |
| 32 | 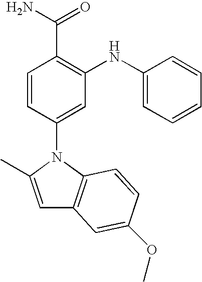 | 4-(5-methoxy-2-methyl-1H-indol-1-yl)-2-(phenylamino)benzamide | 372 |
| 33 | 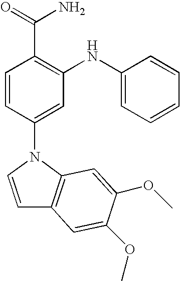 | 4-(5,6-dimethoxy-1H-indol-1-yl)-2-(phenylamino)benzamide | 388 |
| 34 | 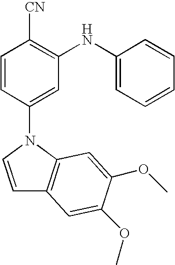 | 4-(5,6-dimethoxy-1H-indol-1-yl)-2-(phenylamino)benzonitrile | 370 |
| 35 | 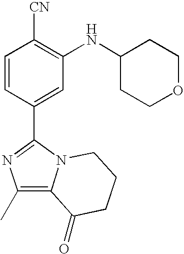 | 4-(1-methyl-8-oxo-5,6,7,8-tetrahydroimidazo[1,5-a]pyridin-3-yl)-2-(tetrahydro-2H-pyran-4-ylamino)benzonitrile | 351 |
| 36 | 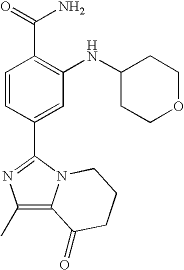 | 4-(1-methyl-8-oxo-5,6,7,8-tetrahydroimidazo[1,5-a]pyridin-3-yl)-2-(tetrahydro-2H-pyran-4-ylamino)benzamide | 369 |
| 37 | 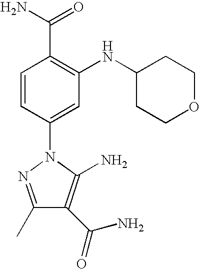 | 5-amino-1-(4-carbamoyl-3-(tetrahydro-2H-pyran-4-ylamino)phenyl)-3-methyl-1H-pyrazole-4-carboxamide | 359 |
| 38 | 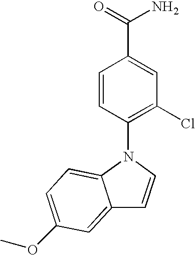 | 3-chloro-4-(5-methoxy-1H-indol-1-yl)benzamide | 301 |
| 39 | 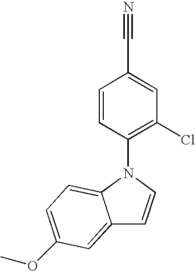 | 3-chloro-4-(5-methoxy-1H-indol-1-yl)benzonitrile | 283 |
| 40 | 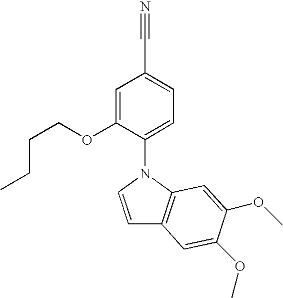 | 3-butoxy-4-(5,6-dimethoxy-1H-indol-1-yl)benzonitrile | 351 |
| 41 | 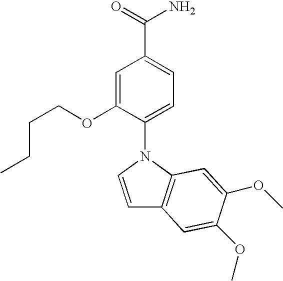 | 3-butoxy-4-(5,6-dimethoxy-1H-indol-1-yl)benzamide | 369 |
| 42 | 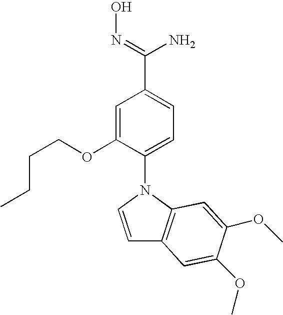 | (Z)-3-butoxy-4-(5,6-dimethoxy-1H-indol-1-yl)-N′-hydroxybenzimidamide | 384 |
| 43 | 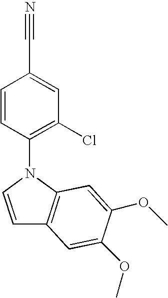 | 3-chloro-4-(5,6-dimethoxy-1H-indol-1-yl)benzonitrile | 313 |
| 44 | 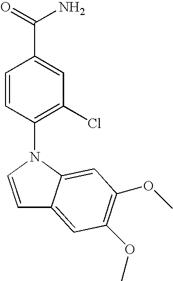 | 3-chloro-4-(5,6-dimethoxy-1H-indol-1-yl)benzamide | 331 |
| 45 | 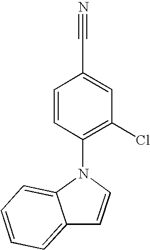 | 3-chloro-4-(1H-indol-1-yl)benzonitrile | 253 |
| 46 | 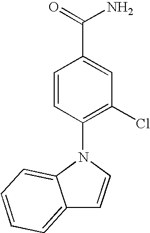 | 3-chloro-4-(1H-indol-1-yl)benzamide | 271 |
| 47 | 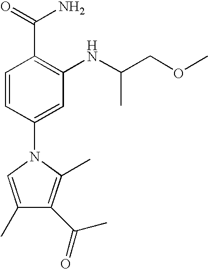 | 4-(3-acetyl-2,4-dimethyl-1H-pyrrol-1-yl)-2-(1-methoxypropan-2-ylamino)benzamide | 344 |
| 48 | 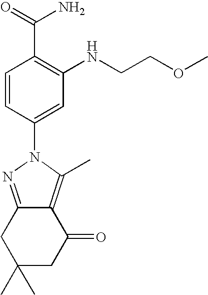 | 2-(2-methoxyethylamino)-4-(3,6,6-trimethyl-4-oxo-4,5,6,7-tetrahydro-2H-indazol-2-yl)benzamide | 371 |
| 49 | 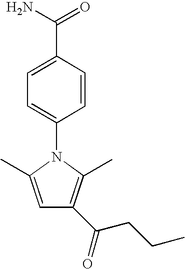 | 4-(3-butyryl-2,5-dimethyl-1H-pyrrol-1-yl)benzamide | 285 |
| 50 | 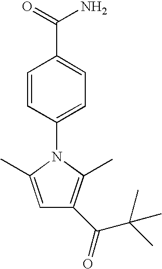 | 4-(2,5-dimethyl-3-pivaloyl-1H-pyrrol-1-yl)benzamide | 299 |
| 51 | 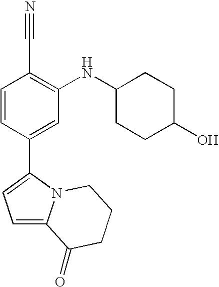 | 2-(4-hydroxycyclohexylamino)-4-(8-oxo-5,6,7,8-tetrahydroindolizin-3-yl)benzonitrile | 350 |
| 52 | 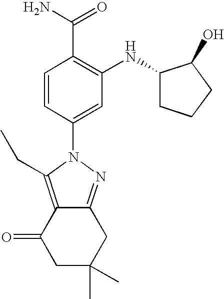 | 4-(3-ethyl-6,6-dimethyl-4-oxo-4,5,6,7-tetrahydro-2H-indazol-2-yl)-2-((1S,2S)-2-hydroxycyclopentylamino)ben-zamide | 410 |
| 53 | 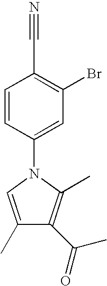 | 4-(3-acetyl-2,4-dimethyl-1H-pyrrol-1-yl)-2-bromobenzonitrile | 317 |
| 54 | 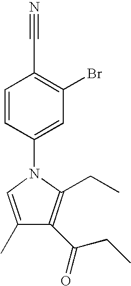 | 2-bromo-4-(2-ethyl-4-methyl-3-propionyl-1H-pyrrol-1-yl)benzonitrile | 345 |
| 55 | 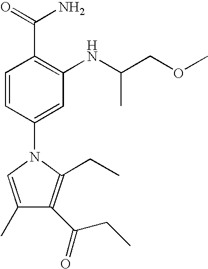 | 4-(2-ethyl-4-methyl-3-propionyl-1H-pyrrol-1-yl)-2-(1-methoxypropan-2-ylamino)benzamide | 372 |
| 56 | 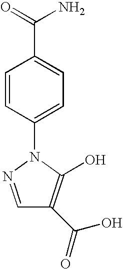 | 1-(4-carbamoylphenyl)-5-hydroxy-1H-pyrazole-4-carboxylic acid | 348 |
| 57 | 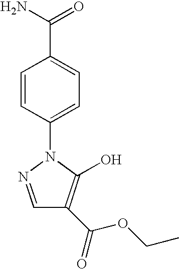 | ethyl 1-(4-carbamoylphenyl)-5-hydroxy-1H-pyrazole-4-carboxylate | 348 |

### Example 32

The compounds listed below in Tables 1-41 are prepared essentially according to the procedures outlined in the above schemes and detailed in the preceding synthetic examples. Thus, the procedures for preparing the following compounds use the same or analogous synthetic techniques with substitution of alternative starting materials as necessary. Suitable variations and alternatives for preparing the following compounds will be readily apparent to those skilled in the art of organic synthesis:

### In each of the following tables 1-41, the various substituents are defined in the following table.

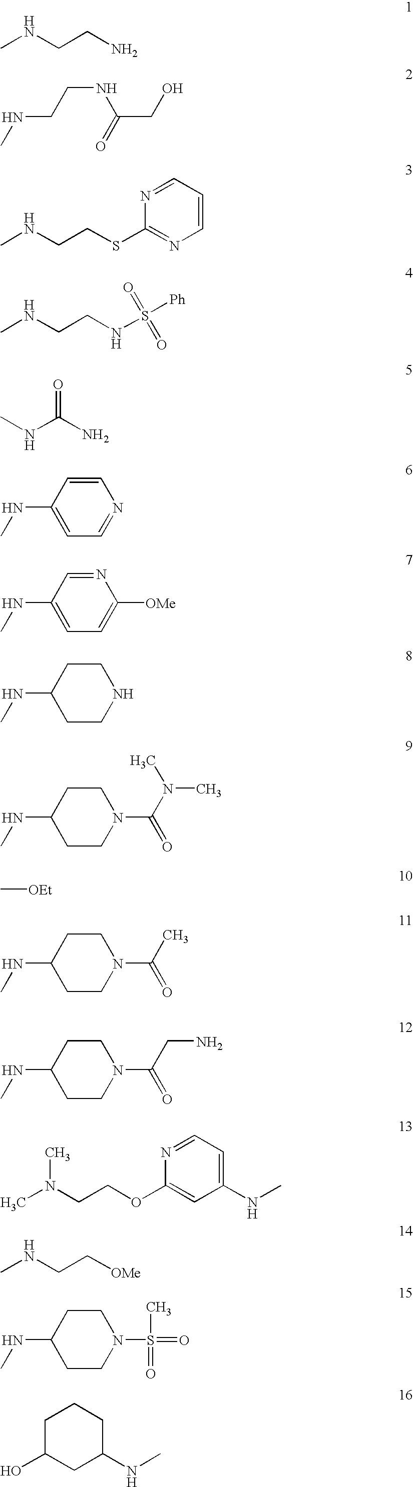

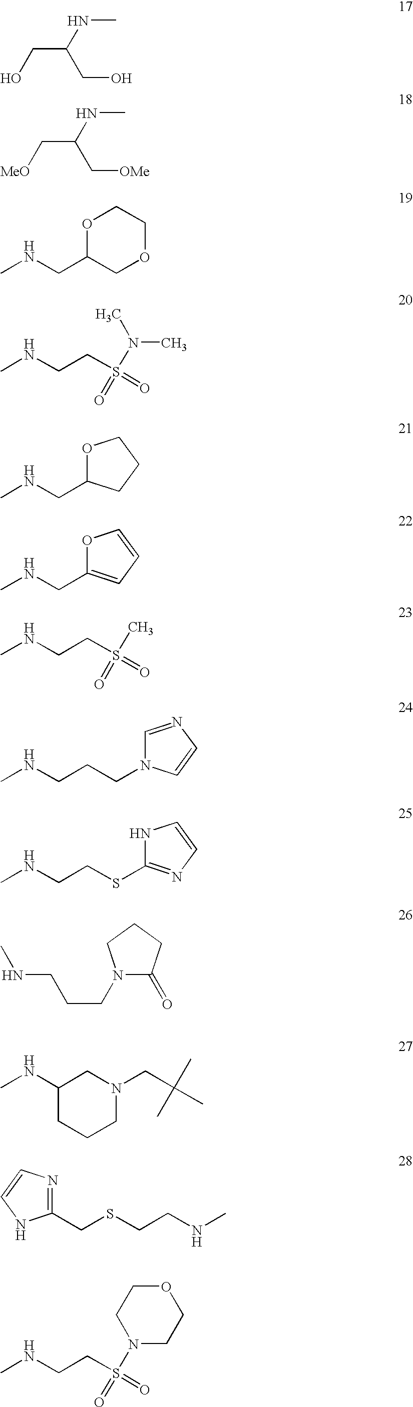

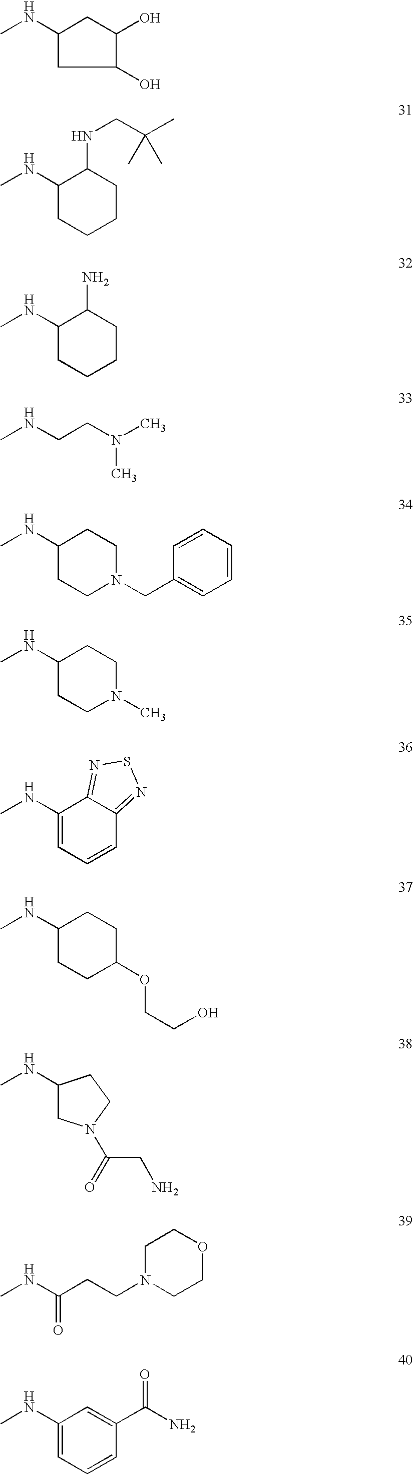

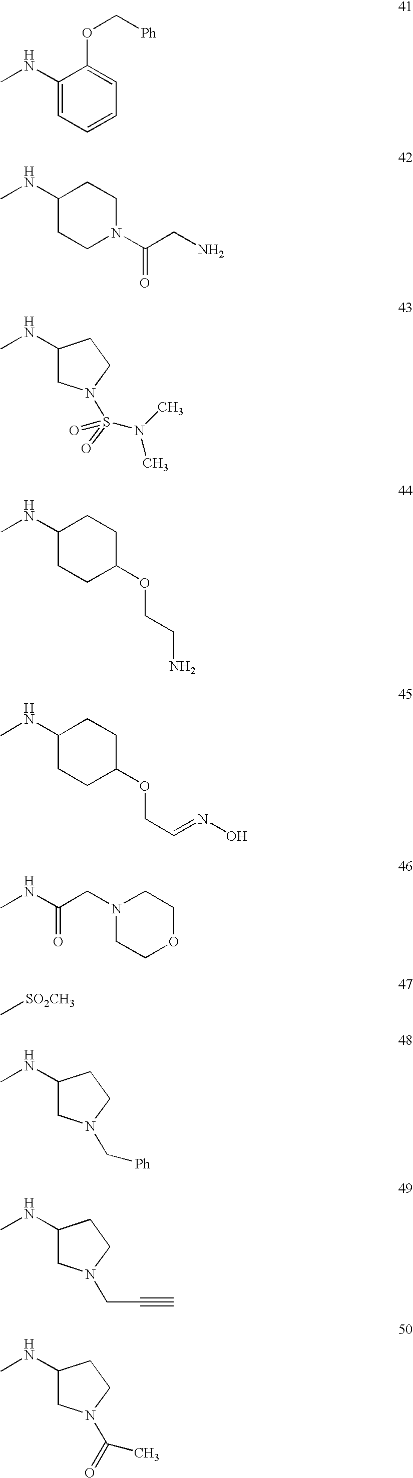

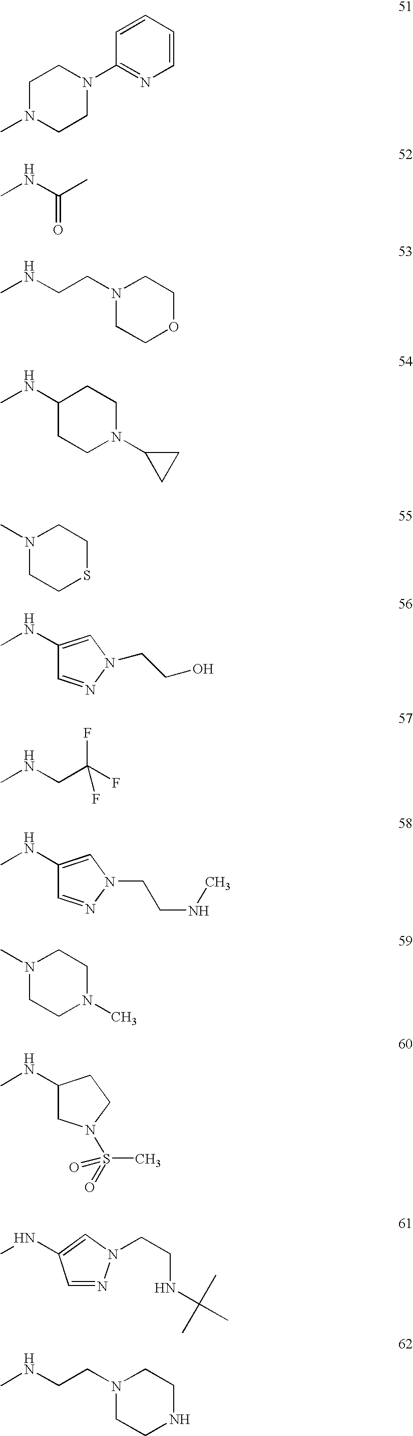

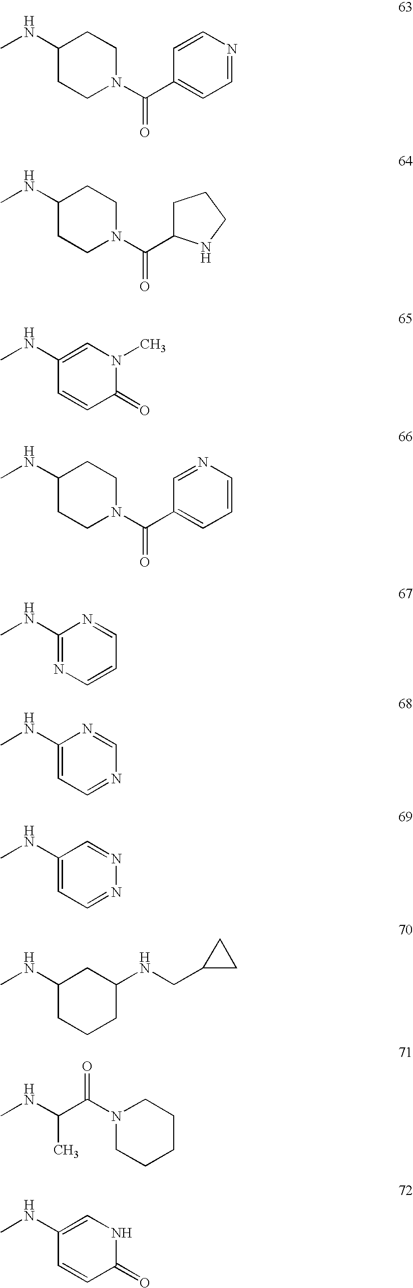

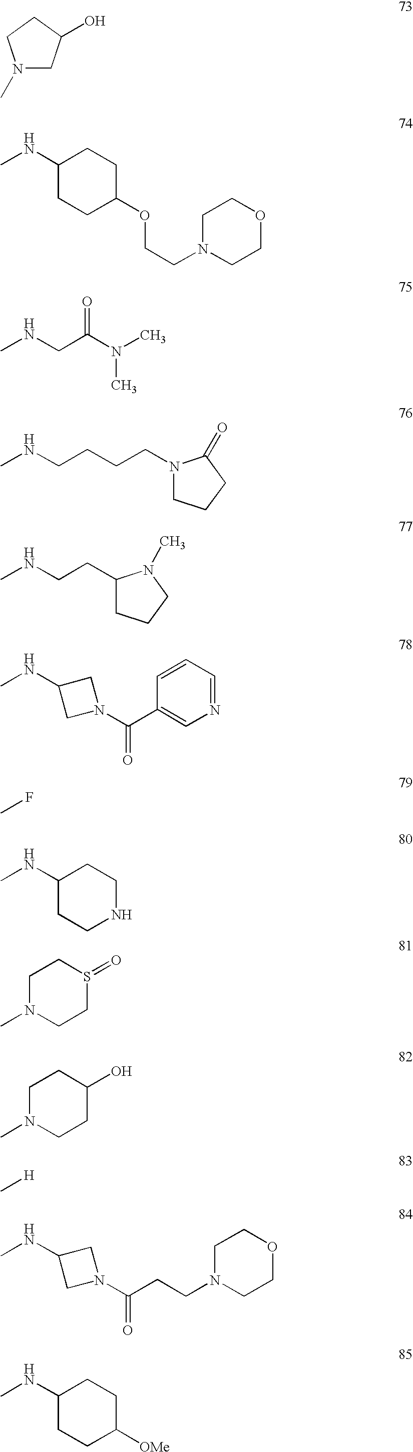

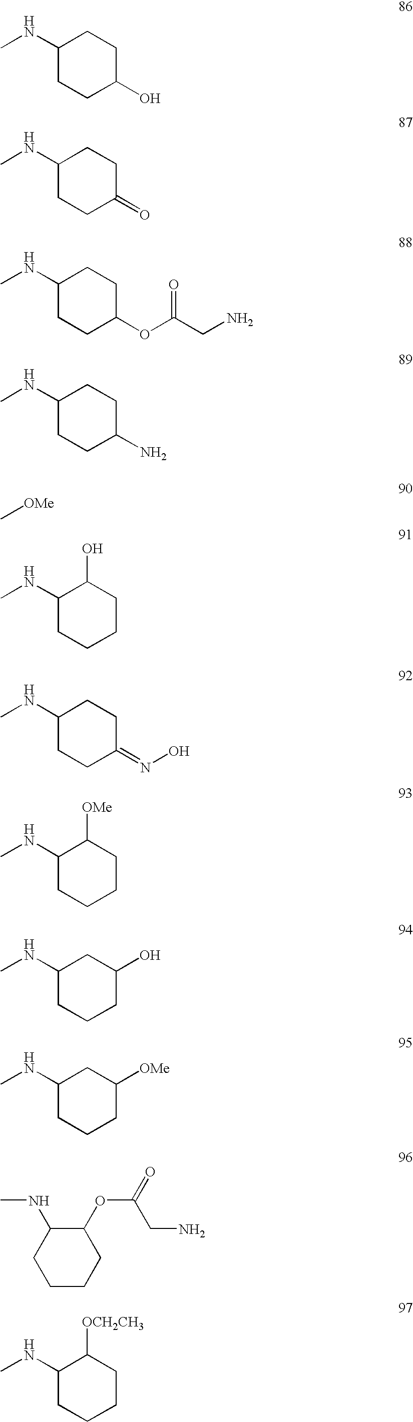

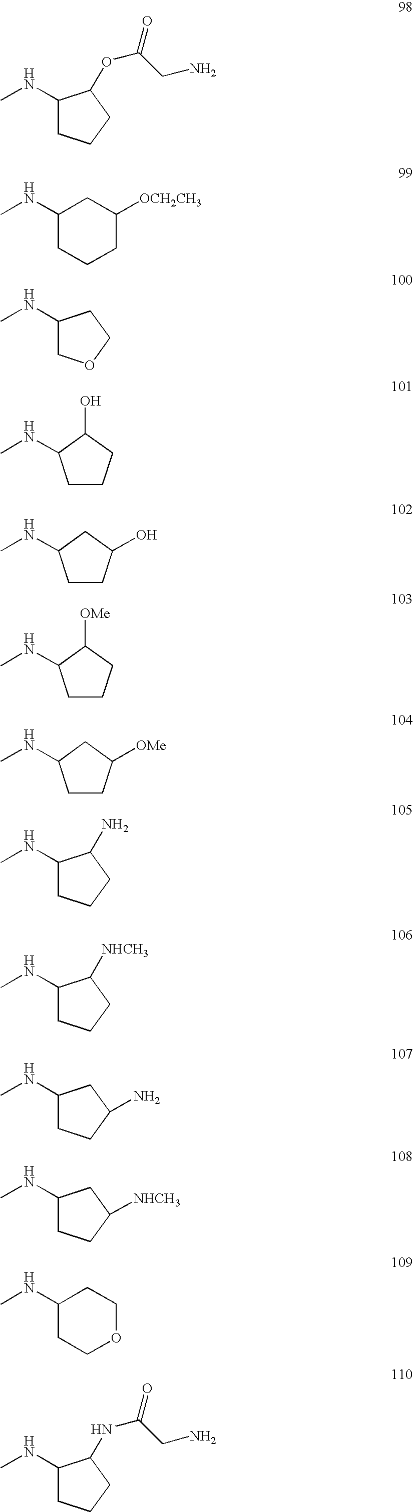

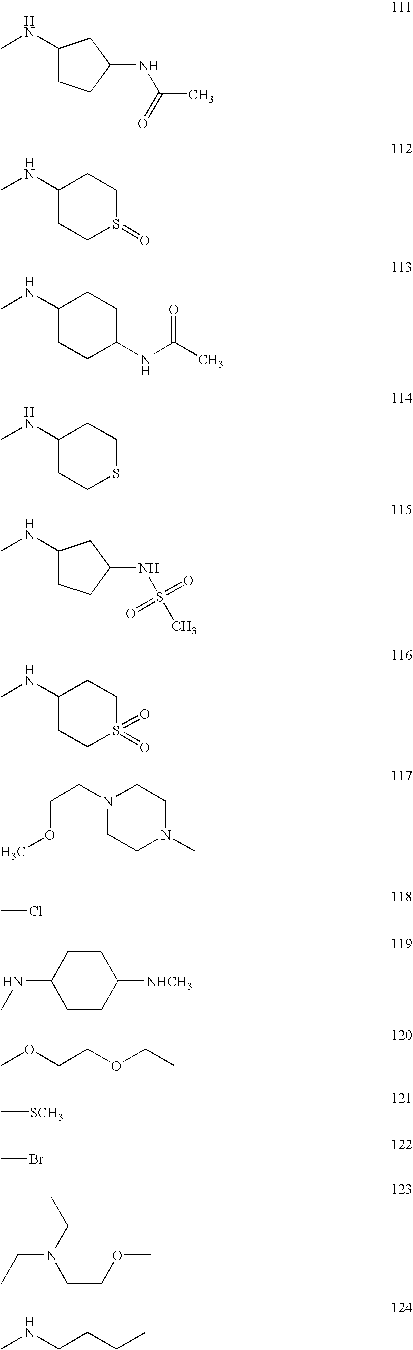

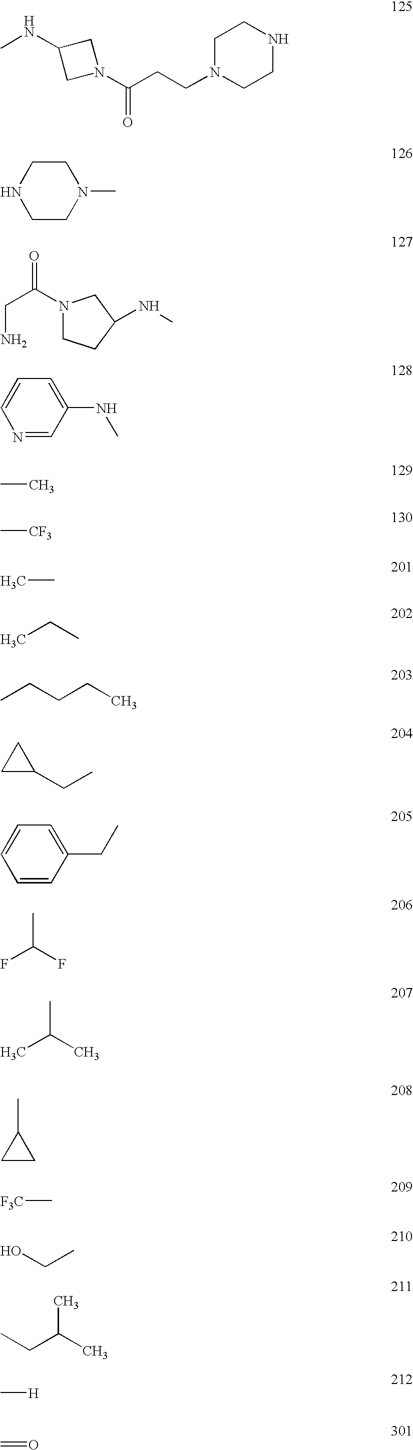

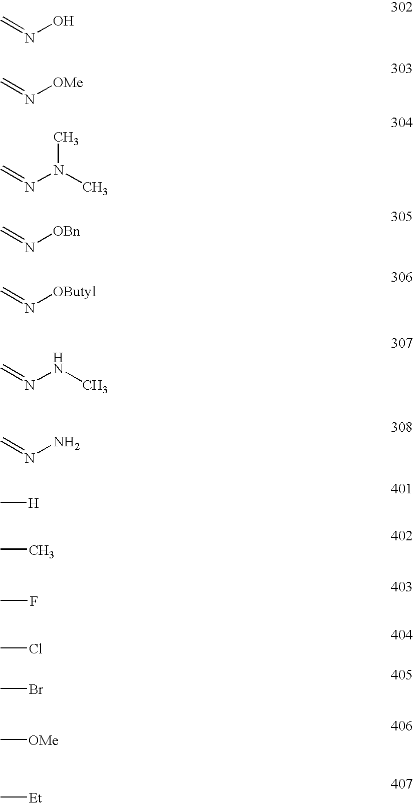

Compounds having the formula:

TABLE 1

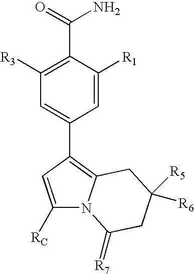

wherein R₁, R₃, R_{C}, R₅, R₆, and R₇ are defined in Table 1:

| Compound | | | | | | |
|---|---|---|---|---|---|---|
| No. | R₁ | R₃ | R_{C} | R₅ | R₆ | R₇ |
| 58 | 402 | 65 | 212 | 212 | 212 | 306 |
| 59 | 407 | 71 | 211 | 202 | 212 | 306 |
| 60 | 404 | 88 | 210 | 201 | 201 | 301 |
| 61 | 401 | 33 | 203 | 202 | 212 | 302 |
| 62 | 404 | 127 | 210 | 201 | 201 | 301 |
| 63 | 402 | 64 | 212 | 201 | 201 | 308 |
| 64 | 402 | 123 | 201 | 201 | 201 | 307 |
| 65 | 401 | 88 | 206 | 202 | 212 | 303 |
| 66 | 401 | 27 | 212 | 202 | 212 | 306 |
| 67 | 405 | 112 | 212 | 201 | 201 | 304 |
| 68 | 404 | 75 | 204 | 202 | 212 | 302 |
| 69 | 406 | 96 | 209 | 201 | 201 | 302 |
| 70 | 401 | 86 | 205 | 201 | 201 | 303 |
| 71 | 401 | 101 | 201 | 212 | 212 | 308 |
| 72 | 402 | 48 | 212 | 202 | 212 | 303 |
| 73 | 401 | 3 | 203 | 202 | 212 | 302 |
| 74 | 404 | 88 | 206 | 212 | 212 | 301 |
| 75 | 402 | 50 | 212 | 202 | 212 | 307 |
| 76 | 405 | 119 | 212 | 212 | 212 | 302 |
| 77 | 402 | 88 | 209 | 212 | 212 | 307 |
| 78 | 401 | 60 | 202 | 201 | 201 | 301 |
| 79 | 405 | 78 | 204 | 201 | 201 | 307 |
| 80 | 404 | 101 | 205 | 202 | 212 | 302 |
| 81 | 401 | 7 | 204 | 201 | 201 | 307 |
| 82 | 405 | 46 | 204 | 202 | 212 | 308 |
| 83 | 406 | 109 | 207 | 201 | 201 | 304 |
| 84 | 407 | 1 | 201 | 212 | 212 | 308 |
| 85 | 401 | 66 | 201 | 212 | 212 | 304 |
| 86 | 401 | 9 | 212 | 212 | 212 | 305 |
| 87 | 403 | 39 | 204 | 212 | 212 | 302 |
| 88 | 401 | 74 | 210 | 201 | 201 | 303 |
| 89 | 401 | 98 | 208 | 201 | 201 | 301 |
| 90 | 401 | 26 | 209 | 212 | 212 | 303 |
| 91 | 401 | 129 | 211 | 202 | 212 | 302 |
| 92 | 401 | 96 | 212 | 201 | 201 | 301 |
| 93 | 401 | 40 | 210 | 202 | 212 | 306 |
| 94 | 401 | 108 | 205 | 212 | 212 | 302 |
| 95 | 401 | 109 | 201 | 212 | 212 | 301 |
| 96 | 405 | 98 | 212 | 201 | 201 | 307 |
| 97 | 401 | 85 | 204 | 212 | 212 | 301 |
| 98 | 404 | 86 | 201 | 202 | 212 | 301 |
| 99 | 402 | 11 | 212 | 212 | 212 | 306 |
| 100 | 401 | 89 | 206 | 202 | 212 | 306 |
| 101 | 405 | 51 | 211 | 201 | 201 | 307 |
| 102 | 401 | 85 | 212 | 212 | 212 | 307 |
| 103 | 403 | 49 | 209 | 201 | 201 | 307 |
| 104 | 401 | 91 | 203 | 201 | 201 | 306 |
| 105 | 401 | 91 | 209 | 212 | 212 | 302 |
| 106 | 406 | 83 | 211 | 212 | 212 | 303 |
| 107 | 401 | 68 | 204 | 212 | 212 | 304 |
| 108 | 403 | 88 | 210 | 202 | 212 | 306 |
| 109 | 405 | 111 | 204 | 202 | 212 | 308 |
| 110 | 404 | 98 | 204 | 212 | 212 | 301 |
| 111 | 407 | 104 | 210 | 202 | 212 | 307 |
| 112 | 403 | 6 | 210 | 202 | 212 | 306 |
| 113 | 402 | 22 | 211 | 201 | 201 | 308 |
| 114 | 407 | 110 | 207 | 201 | 201 | 302 |
| 115 | 401 | 70 | 205 | 201 | 201 | 302 |
| 116 | 403 | 44 | 202 | 201 | 201 | 307 |
| 117 | 405 | 99 | 203 | 202 | 212 | 307 |
| 118 | 401 | 23 | 205 | 202 | 212 | 301 |
| 119 | 401 | 15 | 211 | 212 | 212 | 302 |
| 120 | 402 | 98 | 205 | 201 | 201 | 302 |
| 121 | 402 | 121 | 209 | 202 | 212 | 308 |
| 122 | 403 | 82 | 209 | 202 | 212 | 302 |
| 123 | 407 | 126 | 212 | 202 | 212 | 307 |
| 124 | 406 | 57 | 202 | 212 | 212 | 308 |
| 125 | 403 | 93 | 205 | 212 | 212 | 307 |
| 126 | 401 | 88 | 211 | 201 | 201 | 306 |
| 127 | 401 | 95 | 204 | 201 | 201 | 308 |
| 128 | 405 | 30 | 210 | 201 | 201 | 305 |
| 129 | 406 | 88 | 208 | 212 | 212 | 301 |
| 130 | 401 | 85 | 210 | 202 | 212 | 303 |
| 131 | 405 | 91 | 201 | 201 | 201 | 305 |
| 132 | 401 | 122 | 201 | 201 | 201 | 308 |
| 133 | 407 | 35 | 209 | 202 | 212 | 301 |
| 134 | 401 | 47 | 203 | 202 | 212 | 306 |
| 135 | 405 | 107 | 204 | 201 | 201 | 302 |
| 136 | 401 | 43 | 212 | 202 | 212 | 308 |
| 137 | 406 | 61 | 202 | 202 | 212 | 301 |
| 138 | 406 | 97 | 210 | 201 | 201 | 304 |
| 139 | 401 | 41 | 202 | 202 | 212 | 302 |
| 140 | 406 | 86 | 204 | 212 | 212 | 304 |
| 141 | 406 | 88 | 204 | 202 | 212 | 306 |
| 142 | 401 | 109 | 204 | 212 | 212 | 303 |
| 143 | 407 | 91 | 206 | 212 | 212 | 307 |
| 144 | 401 | 12 | 207 | 201 | 201 | 303 |
| 145 | 401 | 36 | 212 | 201 | 201 | 301 |
| 146 | 404 | 109 | 204 | 201 | 201 | 303 |
| 147 | 401 | 16 | 205 | 201 | 201 | 306 |
| 148 | 401 | 10 | 211 | 202 | 212 | 308 |
| 149 | 402 | 37 | 204 | 202 | 212 | 306 |
| 150 | 401 | 92 | 204 | 212 | 212 | 301 |
| 151 | 404 | 117 | 211 | 202 | 212 | 302 |
| 152 | 403 | 100 | 212 | 212 | 212 | 308 |
| 153 | 404 | 101 | 207 | 212 | 212 | 301 |
| 154 | 401 | 101 | 212 | 202 | 212 | 307 |
| 155 | 405 | 98 | 208 | 201 | 201 | 308 |
| 156 | 407 | 113 | 210 | 201 | 201 | 307 |
| 157 | 402 | 2 | 201 | 201 | 201 | 308 |
| 158 | 406 | 31 | 208 | 212 | 212 | 305 |
| 159 | 401 | 32 | 211 | 212 | 212 | 301 |
| 160 | 404 | 55 | 204 | 201 | 201 | 302 |
| 161 | 401 | 96 | 211 | 201 | 201 | 301 |
| 162 | 405 | 38 | 206 | 201 | 201 | 305 |
| 163 | 402 | 109 | 207 | 201 | 201 | 302 |
| 164 | 403 | 116 | 205 | 202 | 212 | 301 |
| 165 | 401 | 85 | 211 | 212 | 212 | 301 |
| 166 | 407 | 115 | 206 | 201 | 201 | 308 |
| 167 | 402 | 52 | 201 | 202 | 212 | 306 |
| 168 | 405 | 25 | 204 | 202 | 212 | 306 |
| 169 | 405 | 86 | 210 | 212 | 212 | 303 |
| 170 | 406 | 42 | 205 | 212 | 212 | 308 |
| 171 | 403 | 102 | 201 | 201 | 201 | 308 |
| 172 | 401 | 101 | 211 | 201 | 201 | 306 |
| 173 | 405 | 109 | 212 | 201 | 201 | 305 |
| 174 | 407 | 63 | 210 | 202 | 212 | 308 |
| 175 | 407 | 81 | 210 | 212 | 212 | 301 |
| 176 | 405 | 128 | 212 | 201 | 201 | 303 |
| 177 | 403 | 79 | 206 | 212 | 212 | 301 |
| 178 | 402 | 85 | 204 | 212 | 212 | 308 |
| 179 | 406 | 85 | 204 | 202 | 212 | 301 |
| 180 | 404 | 96 | 201 | 201 | 201 | 304 |
| 181 | 401 | 103 | 205 | 201 | 201 | 301 |
| 182 | 401 | 114 | 212 | 202 | 212 | 304 |
| 183 | 403 | 56 | 209 | 202 | 212 | 304 |
| 184 | 401 | 77 | 204 | 212 | 212 | 301 |
| 185 | 401 | 24 | 204 | 201 | 201 | 301 |
| 186 | 404 | 120 | 201 | 201 | 201 | 304 |
| 187 | 401 | 109 | 207 | 201 | 201 | 306 |
| 188 | 401 | 53 | 204 | 201 | 201 | 303 |
| 189 | 401 | 84 | 203 | 212 | 212 | 303 |
| 190 | 401 | 87 | 206 | 202 | 212 | 302 |
| 191 | 401 | 96 | 212 | 212 | 212 | 307 |
| 192 | 406 | 17 | 207 | 201 | 201 | 308 |
| 193 | 401 | 130 | 206 | 202 | 212 | 306 |
| 194 | 404 | 91 | 212 | 201 | 201 | 304 |
| 195 | 401 | 62 | 205 | 212 | 212 | 303 |
| 196 | 401 | 72 | 204 | 202 | 212 | 305 |
| 197 | 407 | 67 | 211 | 201 | 201 | 302 |
| 198 | 404 | 90 | 205 | 212 | 212 | 305 |
| 199 | 401 | 80 | 204 | 212 | 212 | 306 |
| 200 | 403 | 59 | 206 | 201 | 201 | 306 |
| 201 | 402 | 58 | 202 | 212 | 212 | 306 |
| 202 | 401 | 45 | 205 | 201 | 201 | 303 |
| 203 | 401 | 73 | 210 | 202 | 212 | 308 |
| 204 | 401 | 118 | 203 | 212 | 212 | 306 |
| 205 | 404 | 8 | 204 | 212 | 212 | 301 |
| 206 | 407 | 125 | 201 | 212 | 212 | 308 |
| 207 | 403 | 96 | 201 | 201 | 201 | 308 |
| 208 | 407 | 34 | 205 | 201 | 201 | 304 |
| 209 | 401 | 106 | 205 | 212 | 212 | 307 |
| 210 | 401 | 18 | 202 | 202 | 212 | 306 |
| 211 | 406 | 13 | 204 | 201 | 201 | 302 |
| 212 | 401 | 98 | 209 | 201 | 201 | 307 |
| 213 | 407 | 54 | 212 | 201 | 201 | 302 |
| 214 | 404 | 91 | 210 | 201 | 201 | 308 |
| 215 | 401 | 28 | 212 | 202 | 212 | 302 |
| 216 | 401 | 86 | 212 | 212 | 212 | 301 |
| 217 | 406 | 96 | 210 | 212 | 212 | 305 |
| 218 | 402 | 19 | 202 | 201 | 201 | 308 |
| 219 | 401 | 105 | 212 | 202 | 212 | 306 |
| 220 | 406 | 96 | 204 | 201 | 201 | 308 |
| 221 | 401 | 124 | 203 | 201 | 201 | 308 |
| 222 | 406 | 4 | 201 | 201 | 201 | 304 |
| 223 | 407 | 76 | 202 | 202 | 212 | 306 |
| 224 | 403 | 21 | 212 | 202 | 212 | 301 |
| 225 | 403 | 5 | 205 | 201 | 201 | 302 |
| 226 | 401 | 101 | 203 | 201 | 201 | 301 |
| 227 | 402 | 20 | 201 | 201 | 201 | 305 |
| 228 | 401 | 69 | 210 | 201 | 201 | 303 |
| 229 | 403 | 29 | 210 | 202 | 212 | 306 |
| 230 | 401 | 94 | 201 | 212 | 212 | 304 |
| 231 | 401 | 14 | 203 | 201 | 201 | 308 |
| 232 | 407 | 86 | 201 | 212 | 212 | 302 |

Compounds having the formula:

TABLE 2

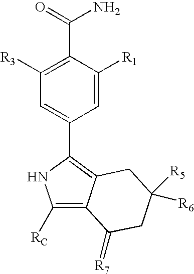

wherein R₁, R₃, R_{C}, R₅, R₆, and R₇ are defined in Table 2:

| Compound | | | | | | |
|---|---|---|---|---|---|---|
| No. | R₁ | R₃ | R_{C} | R₅ | R₆ | R₇ |
| 233 | 402 | 32 | 211 | 212 | 212 | 301 |
| 234 | 407 | 86 | 210 | 212 | 212 | 303 |
| 235 | 404 | 86 | 204 | 212 | 212 | 304 |
| 236 | 404 | 96 | 209 | 201 | 201 | 302 |
| 237 | 402 | 27 | 212 | 202 | 212 | 306 |
| 238 | 402 | 109 | 207 | 201 | 201 | 306 |
| 239 | 401 | 67 | 211 | 201 | 201 | 302 |
| 240 | 401 | 39 | 204 | 212 | 212 | 302 |
| 241 | 405 | 96 | 201 | 201 | 201 | 304 |
| 242 | 404 | 37 | 204 | 202 | 212 | 306 |
| 243 | 406 | 54 | 212 | 201 | 201 | 302 |
| 244 | 401 | 55 | 204 | 201 | 201 | 302 |
| 245 | 401 | 85 | 204 | 202 | 212 | 301 |
| 246 | 402 | 106 | 205 | 212 | 212 | 307 |
| 247 | 401 | 96 | 204 | 201 | 201 | 308 |
| 248 | 404 | 88 | 210 | 202 | 212 | 306 |
| 249 | 402 | 70 | 205 | 201 | 201 | 302 |
| 250 | 405 | 109 | 207 | 201 | 201 | 304 |
| 251 | 402 | 6 | 210 | 202 | 212 | 306 |
| 252 | 401 | 58 | 202 | 212 | 212 | 306 |
| 253 | 405 | 105 | 212 | 202 | 212 | 306 |
| 254 | 404 | 25 | 204 | 202 | 212 | 306 |
| 255 | 401 | 101 | 201 | 212 | 212 | 308 |
| 256 | 405 | 74 | 210 | 201 | 201 | 303 |
| 257 | 406 | 116 | 205 | 202 | 212 | 301 |
| 258 | 407 | 119 | 212 | 212 | 212 | 302 |
| 259 | 401 | 104 | 210 | 202 | 212 | 307 |
| 260 | 401 | 21 | 212 | 202 | 212 | 301 |
| 261 | 403 | 93 | 205 | 212 | 212 | 307 |
| 262 | 401 | 48 | 212 | 202 | 212 | 303 |
| 263 | 401 | 96 | 212 | 201 | 201 | 301 |
| 264 | 401 | 69 | 210 | 201 | 201 | 303 |
| 265 | 401 | 117 | 211 | 202 | 212 | 302 |
| 266 | 401 | 110 | 207 | 201 | 201 | 302 |
| 267 | 401 | 78 | 204 | 201 | 201 | 307 |
| 268 | 401 | 34 | 205 | 201 | 201 | 304 |
| 269 | 401 | 53 | 204 | 201 | 201 | 303 |
| 270 | 405 | 19 | 202 | 201 | 201 | 308 |
| 271 | 401 | 2 | 201 | 201 | 201 | 308 |
| 272 | 404 | 96 | 212 | 212 | 212 | 307 |
| 273 | 402 | 107 | 204 | 201 | 201 | 302 |
| 274 | 401 | 15 | 211 | 212 | 212 | 302 |
| 275 | 405 | 85 | 204 | 212 | 212 | 301 |
| 276 | 401 | 65 | 212 | 212 | 212 | 306 |
| 277 | 403 | 101 | 212 | 202 | 212 | 307 |
| 278 | 401 | 98 | 205 | 201 | 201 | 302 |
| 279 | 401 | 88 | 208 | 212 | 212 | 301 |
| 280 | 406 | 66 | 201 | 212 | 212 | 304 |
| 281 | 401 | 97 | 210 | 201 | 201 | 304 |
| 282 | 403 | 101 | 205 | 202 | 212 | 302 |
| 283 | 405 | 79 | 206 | 212 | 212 | 301 |
| 284 | 404 | 36 | 212 | 201 | 201 | 301 |
| 285 | 407 | 42 | 205 | 212 | 212 | 308 |
| 286 | 403 | 61 | 202 | 202 | 212 | 301 |
| 287 | 402 | 109 | 201 | 212 | 212 | 301 |
| 288 | 407 | 43 | 212 | 202 | 212 | 308 |
| 289 | 401 | 118 | 203 | 212 | 212 | 306 |
| 290 | 403 | 101 | 207 | 212 | 212 | 301 |
| 291 | 405 | 85 | 211 | 212 | 212 | 301 |
| 292 | 401 | 17 | 207 | 201 | 201 | 308 |
| 293 | 401 | 35 | 209 | 202 | 212 | 301 |
| 294 | 402 | 11 | 212 | 212 | 212 | 306 |
| 295 | 402 | 108 | 205 | 212 | 212 | 302 |
| 296 | 403 | 88 | 211 | 201 | 201 | 306 |
| 297 | 407 | 98 | 209 | 201 | 201 | 307 |
| 298 | 406 | 122 | 201 | 201 | 201 | 308 |
| 299 | 403 | 125 | 201 | 212 | 212 | 308 |
| 300 | 401 | 1 | 201 | 212 | 212 | 308 |
| 301 | 401 | 51 | 211 | 201 | 201 | 307 |
| 302 | 405 | 91 | 203 | 201 | 201 | 306 |
| 303 | 406 | 90 | 205 | 212 | 212 | 305 |
| 304 | 401 | 7 | 204 | 201 | 201 | 307 |
| 305 | 405 | 86 | 205 | 201 | 201 | 303 |
| 306 | 401 | 96 | 211 | 201 | 201 | 301 |
| 307 | 407 | 41 | 202 | 202 | 212 | 302 |
| 308 | 401 | 28 | 212 | 202 | 212 | 302 |
| 309 | 405 | 86 | 201 | 212 | 212 | 302 |
| 310 | 401 | 10 | 211 | 202 | 212 | 308 |
| 311 | 406 | 16 | 205 | 201 | 201 | 306 |
| 312 | 406 | 77 | 204 | 212 | 212 | 301 |
| 313 | 401 | 52 | 201 | 202 | 212 | 306 |
| 314 | 406 | 109 | 204 | 201 | 201 | 303 |
| 315 | 406 | 94 | 201 | 212 | 212 | 304 |
| 316 | 401 | 113 | 210 | 201 | 201 | 307 |
| 317 | 407 | 75 | 204 | 202 | 212 | 302 |
| 318 | 401 | 112 | 212 | 201 | 201 | 304 |
| 319 | 401 | 84 | 203 | 212 | 212 | 303 |
| 320 | 404 | 85 | 204 | 212 | 212 | 308 |
| 321 | 401 | 56 | 209 | 202 | 212 | 304 |
| 322 | 401 | 109 | 207 | 201 | 201 | 302 |
| 323 | 402 | 124 | 203 | 201 | 201 | 308 |
| 324 | 401 | 126 | 212 | 202 | 212 | 307 |
| 325 | 404 | 8 | 204 | 212 | 212 | 301 |
| 326 | 403 | 101 | 211 | 201 | 201 | 306 |
| 327 | 404 | 88 | 210 | 201 | 201 | 301 |
| 328 | 401 | 85 | 212 | 212 | 212 | 307 |
| 329 | 405 | 64 | 212 | 201 | 201 | 308 |
| 330 | 407 | 98 | 208 | 201 | 201 | 308 |
| 331 | 402 | 38 | 206 | 201 | 201 | 305 |
| 332 | 406 | 109 | 204 | 212 | 212 | 303 |
| 333 | 401 | 80 | 204 | 212 | 212 | 306 |
| 334 | 404 | 18 | 202 | 202 | 212 | 306 |
| 335 | 401 | 60 | 202 | 201 | 201 | 301 |
| 336 | 405 | 24 | 204 | 201 | 201 | 301 |
| 337 | 402 | 76 | 202 | 202 | 212 | 306 |
| 338 | 403 | 46 | 204 | 202 | 212 | 308 |
| 339 | 401 | 33 | 203 | 202 | 212 | 302 |
| 340 | 407 | 31 | 208 | 212 | 212 | 305 |
| 341 | 402 | 87 | 206 | 202 | 212 | 302 |
| 342 | 405 | 71 | 211 | 202 | 212 | 306 |
| 343 | 405 | 115 | 206 | 201 | 201 | 308 |
| 344 | 406 | 96 | 201 | 201 | 201 | 308 |
| 345 | 403 | 98 | 204 | 212 | 212 | 301 |
| 346 | 401 | 123 | 201 | 201 | 201 | 307 |
| 347 | 405 | 101 | 203 | 201 | 201 | 301 |
| 348 | 407 | 9 | 212 | 212 | 212 | 305 |
| 349 | 407 | 91 | 201 | 201 | 201 | 305 |
| 350 | 405 | 111 | 204 | 202 | 212 | 308 |
| 351 | 403 | 72 | 204 | 202 | 212 | 305 |
| 352 | 402 | 44 | 202 | 201 | 201 | 307 |
| 353 | 406 | 128 | 212 | 201 | 201 | 303 |
| 354 | 404 | 127 | 210 | 201 | 201 | 301 |
| 355 | 401 | 91 | 206 | 212 | 212 | 307 |
| 356 | 401 | 49 | 209 | 201 | 201 | 307 |
| 357 | 403 | 30 | 210 | 201 | 201 | 305 |
| 358 | 401 | 82 | 209 | 202 | 212 | 302 |
| 359 | 401 | 130 | 206 | 202 | 212 | 306 |
| 360 | 404 | 91 | 209 | 212 | 212 | 302 |
| 361 | 401 | 63 | 210 | 202 | 212 | 308 |
| 362 | 401 | 40 | 210 | 202 | 212 | 306 |
| 363 | 401 | 29 | 210 | 202 | 212 | 306 |
| 364 | 401 | 85 | 210 | 202 | 212 | 303 |
| 365 | 401 | 73 | 210 | 202 | 212 | 308 |
| 366 | 406 | 4 | 201 | 201 | 201 | 304 |
| 367 | 401 | 86 | 212 | 212 | 212 | 301 |
| 368 | 404 | 81 | 210 | 212 | 212 | 301 |
| 369 | 401 | 98 | 212 | 201 | 201 | 307 |
| 370 | 401 | 88 | 209 | 212 | 212 | 307 |
| 371 | 407 | 57 | 202 | 212 | 212 | 308 |
| 372 | 404 | 86 | 201 | 202 | 212 | 301 |
| 373 | 401 | 20 | 201 | 201 | 201 | 305 |
| 374 | 403 | 14 | 203 | 201 | 201 | 308 |
| 375 | 402 | 68 | 204 | 212 | 212 | 304 |
| 376 | 401 | 88 | 206 | 202 | 212 | 303 |
| 377 | 401 | 99 | 203 | 202 | 212 | 307 |
| 378 | 401 | 114 | 212 | 202 | 212 | 304 |
| 379 | 404 | 47 | 203 | 202 | 212 | 306 |
| 380 | 407 | 12 | 207 | 201 | 201 | 303 |
| 381 | 403 | 62 | 205 | 212 | 212 | 303 |
| 382 | 407 | 109 | 212 | 201 | 201 | 305 |
| 383 | 401 | 89 | 206 | 202 | 212 | 306 |
| 384 | 401 | 92 | 204 | 212 | 212 | 301 |
| 385 | 406 | 45 | 205 | 201 | 201 | 303 |
| 386 | 401 | 22 | 211 | 201 | 201 | 308 |
| 387 | 407 | 59 | 206 | 201 | 201 | 306 |
| 388 | 404 | 96 | 210 | 212 | 212 | 305 |
| 389 | 401 | 120 | 201 | 201 | 201 | 304 |
| 390 | 401 | 50 | 212 | 202 | 212 | 307 |
| 391 | 406 | 103 | 205 | 201 | 201 | 301 |
| 392 | 402 | 83 | 211 | 212 | 212 | 303 |
| 393 | 401 | 13 | 204 | 201 | 201 | 302 |
| 394 | 406 | 91 | 210 | 201 | 201 | 308 |
| 395 | 401 | 91 | 212 | 201 | 201 | 304 |
| 396 | 406 | 129 | 211 | 202 | 212 | 302 |
| 397 | 407 | 5 | 205 | 201 | 201 | 302 |
| 398 | 403 | 3 | 203 | 202 | 212 | 302 |
| 399 | 403 | 102 | 201 | 201 | 201 | 308 |
| 400 | 401 | 26 | 209 | 212 | 212 | 303 |
| 401 | 402 | 88 | 206 | 212 | 212 | 301 |
| 402 | 401 | 23 | 205 | 202 | 212 | 301 |
| 403 | 403 | 88 | 204 | 202 | 212 | 306 |
| 404 | 401 | 121 | 209 | 202 | 212 | 308 |
| 405 | 401 | 98 | 208 | 201 | 201 | 301 |
| 406 | 407 | 100 | 212 | 212 | 212 | 308 |

Compounds having the formula:

TABLE 3

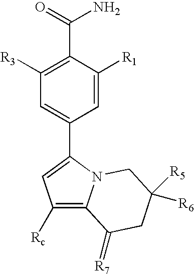

wherein R₁, R₃, R_{C}, R₅, R₆, and R₇ are defined in Table 3:

| Compound | | | | | | |
|---|---|---|---|---|---|---|
| No. | R₁ | R₃ | R_{C} | R₅ | R₆ | R₇ |
| 407 | 402 | 88 | 209 | 212 | 212 | 307 |
| 408 | 407 | 55 | 204 | 201 | 201 | 302 |
| 409 | 404 | 88 | 210 | 201 | 201 | 301 |
| 410 | 401 | 54 | 212 | 201 | 201 | 302 |
| 411 | 404 | 17 | 207 | 201 | 201 | 308 |
| 412 | 402 | 109 | 204 | 201 | 201 | 303 |
| 413 | 402 | 61 | 202 | 202 | 212 | 301 |
| 414 | 401 | 88 | 208 | 212 | 212 | 301 |
| 415 | 401 | 130 | 206 | 202 | 212 | 306 |
| 416 | 405 | 128 | 212 | 201 | 201 | 303 |
| 417 | 404 | 101 | 203 | 201 | 201 | 301 |
| 418 | 406 | 105 | 212 | 202 | 212 | 306 |
| 419 | 401 | 52 | 201 | 202 | 212 | 306 |
| 420 | 401 | 46 | 204 | 202 | 212 | 308 |
| 421 | 402 | 15 | 211 | 212 | 212 | 302 |
| 422 | 401 | 109 | 207 | 201 | 201 | 302 |
| 423 | 404 | 63 | 210 | 202 | 212 | 308 |
| 424 | 402 | 82 | 209 | 202 | 212 | 302 |
| 425 | 405 | 85 | 210 | 202 | 212 | 303 |
| 426 | 402 | 80 | 204 | 212 | 212 | 306 |
| 427 | 401 | 124 | 203 | 201 | 201 | 308 |
| 428 | 405 | 29 | 210 | 202 | 212 | 306 |
| 429 | 404 | 7 | 204 | 201 | 201 | 307 |
| 430 | 401 | 88 | 211 | 201 | 201 | 306 |
| 431 | 405 | 27 | 212 | 202 | 212 | 306 |
| 432 | 406 | 73 | 210 | 202 | 212 | 308 |
| 433 | 407 | 86 | 205 | 201 | 201 | 303 |
| 434 | 401 | 106 | 205 | 212 | 212 | 307 |
| 435 | 401 | 83 | 211 | 212 | 212 | 303 |
| 436 | 403 | 86 | 212 | 212 | 212 | 301 |
| 437 | 401 | 68 | 204 | 212 | 212 | 304 |
| 438 | 401 | 109 | 201 | 212 | 212 | 301 |
| 439 | 401 | 12 | 207 | 201 | 201 | 303 |
| 440 | 401 | 79 | 206 | 212 | 212 | 301 |
| 441 | 401 | 30 | 210 | 201 | 201 | 305 |
| 442 | 401 | 96 | 212 | 201 | 201 | 301 |
| 443 | 401 | 71 | 211 | 202 | 212 | 306 |
| 444 | 401 | 70 | 205 | 201 | 201 | 302 |
| 445 | 405 | 88 | 206 | 202 | 212 | 303 |
| 446 | 401 | 44 | 202 | 201 | 201 | 307 |
| 447 | 404 | 11 | 212 | 212 | 212 | 306 |
| 448 | 402 | 121 | 209 | 202 | 212 | 308 |
| 449 | 401 | 96 | 209 | 201 | 201 | 302 |
| 450 | 405 | 53 | 204 | 201 | 201 | 303 |
| 451 | 401 | 4 | 201 | 201 | 201 | 304 |
| 452 | 403 | 48 | 212 | 202 | 212 | 303 |
| 453 | 401 | 94 | 201 | 212 | 212 | 304 |
| 454 | 401 | 98 | 208 | 201 | 201 | 301 |
| 455 | 406 | 9 | 212 | 212 | 212 | 305 |
| 456 | 401 | 72 | 204 | 202 | 212 | 305 |
| 457 | 403 | 109 | 204 | 212 | 212 | 303 |
| 458 | 405 | 25 | 204 | 202 | 212 | 306 |
| 459 | 404 | 85 | 211 | 212 | 212 | 301 |
| 460 | 407 | 60 | 202 | 201 | 201 | 301 |
| 461 | 403 | 76 | 202 | 202 | 212 | 306 |
| 462 | 402 | 34 | 205 | 201 | 201 | 304 |
| 463 | 407 | 91 | 212 | 201 | 201 | 304 |
| 464 | 401 | 22 | 211 | 201 | 201 | 308 |
| 465 | 403 | 24 | 204 | 201 | 201 | 301 |
| 466 | 405 | 50 | 212 | 202 | 212 | 307 |
| 467 | 401 | 91 | 210 | 201 | 201 | 308 |
| 468 | 401 | 96 | 210 | 212 | 212 | 305 |
| 469 | 402 | 88 | 210 | 202 | 212 | 306 |
| 470 | 402 | 96 | 201 | 201 | 201 | 304 |
| 471 | 403 | 56 | 209 | 202 | 212 | 304 |
| 472 | 407 | 91 | 209 | 212 | 212 | 302 |
| 473 | 406 | 74 | 210 | 201 | 201 | 303 |
| 474 | 403 | 89 | 206 | 202 | 212 | 306 |
| 475 | 401 | 85 | 204 | 202 | 212 | 301 |
| 476 | 401 | 97 | 210 | 201 | 201 | 304 |
| 477 | 405 | 117 | 211 | 202 | 212 | 302 |
| 478 | 406 | 57 | 202 | 212 | 212 | 308 |
| 479 | 401 | 45 | 205 | 201 | 201 | 303 |
| 480 | 405 | 90 | 205 | 212 | 212 | 305 |
| 481 | 401 | 100 | 212 | 212 | 212 | 308 |
| 482 | 407 | 108 | 205 | 212 | 212 | 302 |
| 483 | 401 | 125 | 201 | 212 | 212 | 308 |
| 484 | 405 | 67 | 211 | 201 | 201 | 302 |
| 485 | 401 | 14 | 203 | 201 | 201 | 308 |
| 486 | 406 | 5 | 205 | 201 | 201 | 302 |
| 487 | 406 | 1 | 201 | 212 | 212 | 308 |
| 488 | 401 | 66 | 201 | 212 | 212 | 304 |
| 489 | 406 | 98 | 209 | 201 | 201 | 307 |
| 490 | 406 | 69 | 210 | 201 | 201 | 303 |
| 491 | 401 | 113 | 210 | 201 | 201 | 307 |
| 492 | 407 | 65 | 212 | 212 | 212 | 306 |
| 493 | 401 | 85 | 204 | 212 | 212 | 308 |
| 494 | 401 | 33 | 203 | 202 | 212 | 302 |
| 495 | 404 | 20 | 201 | 201 | 201 | 305 |
| 496 | 401 | 116 | 205 | 202 | 212 | 301 |
| 497 | 401 | 41 | 202 | 202 | 212 | 302 |
| 498 | 402 | 98 | 205 | 201 | 201 | 302 |
| 499 | 401 | 101 | 211 | 201 | 201 | 306 |
| 500 | 404 | 13 | 204 | 201 | 201 | 302 |
| 501 | 403 | 78 | 204 | 201 | 201 | 307 |
| 502 | 404 | 16 | 205 | 201 | 201 | 306 |
| 503 | 401 | 8 | 204 | 212 | 212 | 301 |
| 504 | 405 | 98 | 208 | 201 | 201 | 308 |
| 505 | 407 | 93 | 205 | 212 | 212 | 307 |
| 506 | 402 | 96 | 211 | 201 | 201 | 301 |
| 507 | 406 | 107 | 204 | 201 | 201 | 302 |
| 508 | 401 | 19 | 202 | 201 | 201 | 308 |
| 509 | 404 | 35 | 209 | 202 | 212 | 301 |
| 510 | 401 | 62 | 205 | 212 | 212 | 303 |
| 511 | 405 | 120 | 201 | 201 | 201 | 304 |
| 512 | 402 | 96 | 212 | 212 | 212 | 307 |
| 513 | 403 | 47 | 203 | 202 | 212 | 306 |
| 514 | 401 | 122 | 201 | 201 | 201 | 308 |
| 515 | 407 | 64 | 212 | 201 | 201 | 308 |
| 516 | 402 | 87 | 206 | 202 | 212 | 302 |
| 517 | 405 | 104 | 210 | 202 | 212 | 307 |
| 518 | 405 | 51 | 211 | 201 | 201 | 307 |
| 519 | 406 | 127 | 210 | 201 | 201 | 301 |
| 520 | 403 | 111 | 204 | 202 | 212 | 308 |
| 521 | 401 | 85 | 204 | 212 | 212 | 301 |
| 522 | 405 | 37 | 204 | 202 | 212 | 306 |
| 523 | 407 | 91 | 206 | 212 | 212 | 307 |
| 524 | 407 | 109 | 207 | 201 | 201 | 304 |
| 525 | 405 | 85 | 212 | 212 | 212 | 307 |
| 526 | 403 | 32 | 211 | 212 | 212 | 301 |
| 527 | 402 | 21 | 212 | 202 | 212 | 301 |
| 528 | 406 | 86 | 210 | 212 | 212 | 303 |
| 529 | 404 | 10 | 211 | 202 | 212 | 308 |
| 530 | 401 | 88 | 206 | 212 | 212 | 301 |
| 531 | 401 | 95 | 204 | 201 | 201 | 308 |
| 532 | 403 | 115 | 206 | 201 | 201 | 308 |
| 533 | 401 | 75 | 204 | 202 | 212 | 302 |
| 534 | 401 | 92 | 204 | 212 | 212 | 301 |
| 535 | 404 | 31 | 208 | 212 | 212 | 305 |
| 536 | 401 | 49 | 209 | 201 | 201 | 307 |
| 537 | 401 | 109 | 207 | 201 | 201 | 306 |
| 538 | 401 | 109 | 212 | 201 | 201 | 305 |
| 539 | 401 | 101 | 205 | 202 | 212 | 302 |
| 540 | 401 | 110 | 207 | 201 | 201 | 302 |
| 541 | 406 | 101 | 207 | 212 | 212 | 301 |
| 542 | 401 | 42 | 205 | 212 | 212 | 308 |
| 543 | 404 | 101 | 201 | 212 | 212 | 308 |
| 544 | 401 | 6 | 210 | 202 | 212 | 306 |
| 545 | 401 | 98 | 212 | 201 | 201 | 307 |
| 546 | 407 | 40 | 210 | 202 | 212 | 306 |
| 547 | 404 | 88 | 204 | 202 | 212 | 306 |
| 548 | 401 | 114 | 212 | 202 | 212 | 304 |
| 549 | 403 | 91 | 201 | 201 | 201 | 305 |
| 550 | 402 | 98 | 204 | 212 | 212 | 301 |
| 551 | 401 | 3 | 203 | 202 | 212 | 302 |
| 552 | 401 | 36 | 212 | 201 | 201 | 301 |
| 553 | 401 | 58 | 202 | 212 | 212 | 306 |
| 554 | 404 | 38 | 206 | 201 | 201 | 305 |
| 555 | 407 | 102 | 201 | 201 | 201 | 308 |
| 556 | 403 | 86 | 201 | 202 | 212 | 301 |
| 557 | 407 | 101 | 212 | 202 | 212 | 307 |
| 558 | 401 | 28 | 212 | 202 | 212 | 302 |
| 559 | 401 | 96 | 204 | 201 | 201 | 308 |
| 560 | 406 | 43 | 212 | 202 | 212 | 308 |
| 561 | 401 | 103 | 205 | 201 | 201 | 301 |
| 562 | 407 | 23 | 205 | 202 | 212 | 301 |
| 563 | 404 | 112 | 212 | 201 | 201 | 304 |
| 564 | 401 | 18 | 202 | 202 | 212 | 306 |
| 565 | 401 | 81 | 210 | 212 | 212 | 301 |
| 566 | 406 | 59 | 206 | 201 | 201 | 306 |
| 567 | 402 | 119 | 212 | 212 | 212 | 302 |
| 568 | 401 | 96 | 201 | 201 | 201 | 308 |
| 569 | 406 | 2 | 201 | 201 | 201 | 308 |
| 570 | 401 | 126 | 212 | 202 | 212 | 307 |
| 571 | 406 | 86 | 201 | 212 | 212 | 302 |
| 572 | 407 | 39 | 204 | 212 | 212 | 302 |
| 573 | 403 | 77 | 204 | 212 | 212 | 301 |
| 574 | 403 | 129 | 211 | 202 | 212 | 302 |
| 575 | 401 | 26 | 209 | 212 | 212 | 303 |
| 576 | 402 | 86 | 204 | 212 | 212 | 304 |
| 577 | 401 | 84 | 203 | 212 | 212 | 303 |
| 578 | 403 | 91 | 203 | 201 | 201 | 306 |
| 579 | 401 | 118 | 203 | 212 | 212 | 306 |
| 580 | 401 | 99 | 203 | 202 | 212 | 307 |
| 581 | 407 | 123 | 201 | 201 | 201 | 307 |

Compounds having the formula:

TABLE 4

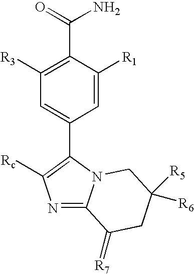

wherein R₁, R₃, R_{C}, R₅, R₆, and R₇ are defined in Table 4.

| Compound | | | | | | |
|---|---|---|---|---|---|---|
| No. | R₁ | R₃ | R_{C} | R₅ | R₆ | R₇ |
| 582 | 402 | 9 | 212 | 212 | 212 | 305 |
| 583 | 407 | 98 | 205 | 201 | 201 | 302 |
| 584 | 404 | 89 | 206 | 202 | 212 | 306 |
| 585 | 401 | 15 | 211 | 212 | 212 | 302 |
| 586 | 404 | 91 | 210 | 201 | 201 | 308 |
| 587 | 402 | 33 | 203 | 202 | 212 | 302 |
| 588 | 402 | 126 | 212 | 202 | 212 | 307 |
| 589 | 401 | 22 | 211 | 201 | 201 | 308 |
| 590 | 401 | 109 | 207 | 201 | 201 | 304 |
| 591 | 405 | 10 | 211 | 202 | 212 | 308 |
| 592 | 404 | 100 | 212 | 212 | 212 | 308 |
| 593 | 406 | 29 | 210 | 202 | 212 | 306 |
| 594 | 401 | 30 | 210 | 201 | 201 | 305 |
| 595 | 401 | 119 | 212 | 212 | 212 | 302 |
| 596 | 402 | 109 | 212 | 201 | 201 | 305 |
| 597 | 401 | 67 | 211 | 201 | 201 | 302 |
| 598 | 404 | 42 | 205 | 212 | 212 | 308 |
| 599 | 402 | 101 | 205 | 202 | 212 | 302 |
| 600 | 405 | 32 | 211 | 212 | 212 | 301 |
| 601 | 402 | 19 | 202 | 201 | 201 | 308 |
| 602 | 401 | 68 | 204 | 212 | 212 | 304 |
| 603 | 405 | 104 | 210 | 202 | 212 | 307 |
| 604 | 404 | 96 | 212 | 201 | 201 | 301 |
| 605 | 401 | 31 | 208 | 212 | 212 | 305 |
| 606 | 405 | 88 | 210 | 201 | 201 | 301 |
| 607 | 406 | 83 | 211 | 212 | 212 | 303 |
| 608 | 407 | 7 | 204 | 201 | 201 | 307 |
| 609 | 401 | 23 | 205 | 202 | 212 | 301 |
| 610 | 401 | 63 | 210 | 202 | 212 | 308 |
| 611 | 403 | 121 | 209 | 202 | 212 | 308 |
| 612 | 401 | 86 | 201 | 202 | 212 | 301 |
| 613 | 401 | 78 | 204 | 201 | 201 | 307 |
| 614 | 401 | 77 | 204 | 212 | 212 | 301 |
| 615 | 401 | 21 | 212 | 202 | 212 | 301 |
| 616 | 401 | 115 | 206 | 201 | 201 | 308 |
| 617 | 401 | 50 | 212 | 202 | 212 | 307 |
| 618 | 401 | 27 | 212 | 202 | 212 | 306 |
| 619 | 401 | 88 | 206 | 202 | 212 | 303 |
| 620 | 405 | 96 | 204 | 201 | 201 | 308 |
| 621 | 401 | 98 | 208 | 201 | 201 | 308 |
| 622 | 404 | 4 | 201 | 201 | 201 | 304 |
| 623 | 402 | 86 | 205 | 201 | 201 | 303 |
| 624 | 401 | 17 | 207 | 201 | 201 | 308 |
| 625 | 405 | 35 | 209 | 202 | 212 | 301 |
| 626 | 401 | 90 | 205 | 212 | 212 | 305 |
| 627 | 403 | 101 | 211 | 201 | 201 | 306 |
| 628 | 401 | 69 | 210 | 201 | 201 | 303 |
| 629 | 401 | 110 | 207 | 201 | 201 | 302 |
| 630 | 406 | 84 | 203 | 212 | 212 | 303 |
| 631 | 401 | 98 | 209 | 201 | 201 | 307 |
| 632 | 403 | 118 | 203 | 212 | 212 | 306 |
| 633 | 405 | 46 | 204 | 202 | 212 | 308 |
| 634 | 404 | 111 | 204 | 202 | 212 | 308 |
| 635 | 407 | 60 | 202 | 201 | 201 | 301 |
| 636 | 403 | 25 | 204 | 202 | 212 | 306 |
| 637 | 402 | 5 | 205 | 201 | 201 | 302 |
| 638 | 407 | 101 | 212 | 202 | 212 | 307 |
| 639 | 401 | 109 | 201 | 212 | 212 | 301 |
| 640 | 403 | 71 | 211 | 202 | 212 | 306 |
| 641 | 405 | 88 | 204 | 202 | 212 | 306 |
| 642 | 401 | 13 | 204 | 201 | 201 | 302 |
| 643 | 401 | 73 | 210 | 202 | 212 | 308 |
| 644 | 402 | 99 | 203 | 202 | 212 | 307 |
| 645 | 402 | 97 | 210 | 201 | 201 | 304 |
| 646 | 403 | 92 | 204 | 212 | 212 | 301 |
| 647 | 407 | 2 | 201 | 201 | 201 | 308 |
| 648 | 406 | 96 | 210 | 212 | 212 | 305 |
| 649 | 403 | 53 | 204 | 201 | 201 | 303 |
| 650 | 401 | 41 | 202 | 202 | 212 | 302 |
| 651 | 401 | 127 | 210 | 201 | 201 | 301 |
| 652 | 405 | 76 | 202 | 202 | 212 | 306 |
| 653 | 406 | 62 | 205 | 212 | 212 | 303 |
| 654 | 401 | 43 | 212 | 202 | 212 | 308 |
| 655 | 405 | 103 | 205 | 201 | 201 | 301 |
| 656 | 401 | 16 | 205 | 201 | 201 | 306 |
| 657 | 407 | 54 | 212 | 201 | 201 | 302 |
| 658 | 401 | 58 | 202 | 212 | 212 | 306 |
| 659 | 405 | 86 | 212 | 212 | 212 | 301 |
| 660 | 401 | 91 | 201 | 201 | 201 | 305 |
| 661 | 406 | 39 | 204 | 212 | 212 | 302 |
| 662 | 406 | 36 | 212 | 201 | 201 | 301 |
| 663 | 401 | 45 | 205 | 201 | 201 | 303 |
| 664 | 406 | 130 | 206 | 202 | 212 | 306 |
| 665 | 406 | 128 | 212 | 201 | 201 | 303 |
| 666 | 401 | 117 | 211 | 202 | 212 | 302 |
| 667 | 407 | 107 | 204 | 201 | 201 | 302 |
| 668 | 401 | 93 | 205 | 212 | 212 | 307 |
| 669 | 401 | 91 | 209 | 212 | 212 | 302 |
| 670 | 404 | 114 | 212 | 202 | 212 | 304 |
| 671 | 401 | 11 | 212 | 212 | 212 | 306 |
| 672 | 401 | 108 | 205 | 212 | 212 | 302 |
| 673 | 402 | 94 | 201 | 212 | 212 | 304 |
| 674 | 401 | 85 | 204 | 212 | 212 | 301 |
| 675 | 404 | 116 | 205 | 202 | 212 | 301 |
| 676 | 403 | 52 | 201 | 202 | 212 | 306 |
| 677 | 404 | 28 | 212 | 202 | 212 | 302 |
| 678 | 401 | 105 | 212 | 202 | 212 | 306 |
| 679 | 405 | 47 | 203 | 202 | 212 | 306 |
| 680 | 407 | 101 | 207 | 212 | 212 | 301 |
| 681 | 402 | 112 | 212 | 201 | 201 | 304 |
| 682 | 406 | 38 | 206 | 201 | 201 | 305 |
| 683 | 401 | 86 | 204 | 212 | 212 | 304 |
| 684 | 404 | 12 | 207 | 201 | 201 | 303 |
| 685 | 401 | 123 | 201 | 201 | 201 | 307 |
| 686 | 405 | 18 | 202 | 202 | 212 | 306 |
| 687 | 402 | 64 | 212 | 201 | 201 | 308 |
| 688 | 403 | 95 | 204 | 201 | 201 | 308 |
| 689 | 401 | 82 | 209 | 202 | 212 | 302 |
| 690 | 407 | 109 | 204 | 212 | 212 | 303 |
| 691 | 402 | 74 | 210 | 201 | 201 | 303 |
| 692 | 405 | 85 | 211 | 212 | 212 | 301 |
| 693 | 405 | 55 | 204 | 201 | 201 | 302 |
| 694 | 406 | 66 | 201 | 212 | 212 | 304 |
| 695 | 403 | 85 | 212 | 212 | 212 | 307 |
| 696 | 401 | 91 | 203 | 201 | 201 | 306 |
| 697 | 405 | 98 | 204 | 212 | 212 | 301 |
| 698 | 407 | 88 | 206 | 212 | 212 | 301 |
| 699 | 407 | 3 | 203 | 202 | 212 | 302 |
| 700 | 405 | 8 | 204 | 212 | 212 | 301 |
| 701 | 403 | 59 | 206 | 201 | 201 | 306 |
| 702 | 402 | 96 | 201 | 201 | 201 | 304 |
| 703 | 406 | 6 | 210 | 202 | 212 | 306 |
| 704 | 404 | 14 | 203 | 201 | 201 | 308 |
| 705 | 401 | 122 | 201 | 201 | 201 | 308 |
| 706 | 401 | 96 | 212 | 212 | 212 | 307 |
| 707 | 403 | 51 | 211 | 201 | 201 | 307 |
| 708 | 401 | 85 | 210 | 202 | 212 | 303 |
| 709 | 401 | 129 | 211 | 202 | 212 | 302 |
| 710 | 404 | 85 | 204 | 202 | 212 | 301 |
| 711 | 401 | 37 | 204 | 202 | 212 | 306 |
| 712 | 401 | 87 | 206 | 202 | 212 | 302 |
| 713 | 401 | 40 | 210 | 202 | 212 | 306 |
| 714 | 401 | 20 | 201 | 201 | 201 | 305 |
| 715 | 401 | 98 | 212 | 201 | 201 | 307 |
| 716 | 406 | 88 | 208 | 212 | 212 | 301 |
| 717 | 401 | 124 | 203 | 201 | 201 | 308 |
| 718 | 404 | 56 | 209 | 202 | 212 | 304 |
| 719 | 401 | 80 | 204 | 212 | 212 | 306 |
| 720 | 401 | 86 | 210 | 212 | 212 | 303 |
| 721 | 407 | 109 | 207 | 201 | 201 | 306 |
| 722 | 404 | 113 | 210 | 201 | 201 | 307 |
| 723 | 401 | 96 | 211 | 201 | 201 | 301 |
| 724 | 403 | 88 | 211 | 201 | 201 | 306 |
| 725 | 402 | 106 | 205 | 212 | 212 | 307 |
| 726 | 401 | 65 | 212 | 212 | 212 | 306 |
| 727 | 401 | 48 | 212 | 202 | 212 | 303 |
| 728 | 401 | 125 | 201 | 212 | 212 | 308 |
| 729 | 404 | 44 | 202 | 201 | 201 | 307 |
| 730 | 407 | 85 | 204 | 212 | 212 | 308 |
| 731 | 403 | 96 | 209 | 201 | 201 | 302 |
| 732 | 407 | 88 | 209 | 212 | 212 | 307 |
| 733 | 401 | 120 | 201 | 201 | 201 | 304 |
| 734 | 401 | 102 | 201 | 201 | 201 | 308 |
| 735 | 406 | 91 | 212 | 201 | 201 | 304 |
| 736 | 401 | 101 | 203 | 201 | 201 | 301 |
| 737 | 407 | 109 | 204 | 201 | 201 | 303 |
| 738 | 404 | 86 | 201 | 212 | 212 | 302 |
| 739 | 401 | 101 | 201 | 212 | 212 | 308 |
| 740 | 401 | 91 | 206 | 212 | 212 | 307 |
| 741 | 406 | 96 | 201 | 201 | 201 | 308 |
| 742 | 402 | 24 | 204 | 201 | 201 | 301 |
| 743 | 401 | 72 | 204 | 202 | 212 | 305 |
| 744 | 406 | 75 | 204 | 202 | 212 | 302 |
| 745 | 401 | 1 | 201 | 212 | 212 | 308 |
| 746 | 406 | 49 | 209 | 201 | 201 | 307 |
| 747 | 407 | 61 | 202 | 202 | 212 | 301 |
| 748 | 403 | 109 | 207 | 201 | 201 | 302 |
| 749 | 403 | 88 | 210 | 202 | 212 | 306 |
| 750 | 401 | 26 | 209 | 212 | 212 | 303 |
| 751 | 402 | 79 | 206 | 212 | 212 | 301 |
| 752 | 401 | 81 | 210 | 212 | 212 | 301 |
| 753 | 403 | 70 | 205 | 201 | 201 | 302 |
| 754 | 401 | 57 | 202 | 212 | 212 | 308 |
| 755 | 401 | 98 | 208 | 201 | 201 | 301 |
| 756 | 407 | 34 | 205 | 201 | 201 | 304 |

Compounds having the formula:

TABLE 5

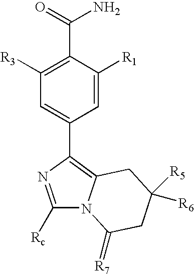

wherein R₁, R₃, R_{C}, R₅, R₆, and R₇ are defined in Table 5:

| Compound | | | | | | |
|---|---|---|---|---|---|---|
| No. | R₁ | R₃ | R_{C} | R₅ | R₆ | R₇ |
| 757 | 402 | 98 | 205 | 201 | 201 | 302 |
| 758 | 407 | 40 | 210 | 202 | 212 | 306 |
| 759 | 404 | 115 | 206 | 201 | 201 | 308 |
| 760 | 401 | 125 | 201 | 212 | 212 | 308 |
| 761 | 404 | 130 | 206 | 202 | 212 | 306 |
| 762 | 402 | 91 | 212 | 201 | 201 | 304 |
| 763 | 402 | 96 | 210 | 212 | 212 | 305 |
| 764 | 401 | 96 | 211 | 201 | 201 | 301 |
| 765 | 401 | 31 | 208 | 212 | 212 | 305 |
| 766 | 405 | 120 | 201 | 201 | 201 | 304 |
| 767 | 404 | 100 | 212 | 212 | 212 | 308 |
| 768 | 406 | 97 | 210 | 201 | 201 | 304 |
| 769 | 401 | 96 | 209 | 201 | 201 | 302 |
| 770 | 401 | 118 | 203 | 212 | 212 | 306 |
| 771 | 402 | 88 | 210 | 201 | 201 | 301 |
| 772 | 401 | 69 | 210 | 201 | 201 | 303 |
| 773 | 404 | 88 | 208 | 212 | 212 | 301 |
| 774 | 402 | 98 | 208 | 201 | 201 | 308 |
| 775 | 405 | 85 | 204 | 202 | 212 | 301 |
| 776 | 402 | 35 | 209 | 202 | 212 | 301 |
| 777 | 401 | 52 | 201 | 202 | 212 | 306 |
| 778 | 405 | 106 | 205 | 212 | 212 | 307 |
| 779 | 404 | 85 | 211 | 212 | 212 | 301 |
| 780 | 401 | 96 | 204 | 201 | 201 | 308 |
| 781 | 405 | 50 | 212 | 202 | 212 | 307 |
| 782 | 406 | 67 | 211 | 201 | 201 | 302 |
| 783 | 407 | 71 | 211 | 202 | 212 | 306 |
| 784 | 401 | 45 | 205 | 201 | 201 | 303 |
| 785 | 401 | 38 | 206 | 201 | 201 | 305 |
| 786 | 403 | 96 | 212 | 212 | 212 | 307 |
| 787 | 401 | 34 | 205 | 201 | 201 | 304 |
| 788 | 401 | 68 | 204 | 212 | 212 | 304 |
| 789 | 401 | 129 | 211 | 202 | 212 | 302 |
| 790 | 401 | 102 | 201 | 201 | 201 | 308 |
| 791 | 401 | 109 | 207 | 201 | 201 | 306 |
| 792 | 401 | 30 | 210 | 201 | 201 | 305 |
| 793 | 401 | 48 | 212 | 202 | 212 | 303 |
| 794 | 401 | 79 | 206 | 212 | 212 | 301 |
| 795 | 405 | 32 | 211 | 212 | 212 | 301 |
| 796 | 401 | 46 | 204 | 202 | 212 | 308 |
| 797 | 404 | 20 | 201 | 201 | 201 | 305 |
| 798 | 402 | 7 | 204 | 201 | 201 | 307 |
| 799 | 401 | 98 | 209 | 201 | 201 | 307 |
| 800 | 405 | 74 | 210 | 201 | 201 | 303 |
| 801 | 401 | 108 | 205 | 212 | 212 | 302 |
| 802 | 403 | 111 | 204 | 202 | 212 | 308 |
| 803 | 401 | 61 | 202 | 202 | 212 | 301 |
| 804 | 401 | 16 | 205 | 201 | 201 | 306 |
| 805 | 406 | 14 | 203 | 201 | 201 | 308 |
| 806 | 401 | 43 | 212 | 202 | 212 | 308 |
| 807 | 403 | 49 | 209 | 201 | 201 | 307 |
| 808 | 405 | 93 | 205 | 212 | 212 | 307 |
| 809 | 404 | 10 | 211 | 202 | 212 | 308 |
| 810 | 407 | 78 | 204 | 201 | 201 | 307 |
| 811 | 403 | 92 | 204 | 212 | 212 | 301 |
| 812 | 402 | 85 | 212 | 212 | 212 | 307 |
| 813 | 407 | 13 | 204 | 201 | 201 | 302 |
| 814 | 401 | 62 | 205 | 212 | 212 | 303 |
| 815 | 403 | 15 | 211 | 212 | 212 | 302 |
| 816 | 405 | 89 | 206 | 202 | 212 | 306 |
| 817 | 401 | 76 | 202 | 202 | 212 | 306 |
| 818 | 401 | 109 | 207 | 201 | 201 | 302 |
| 819 | 402 | 95 | 204 | 201 | 201 | 308 |
| 820 | 402 | 29 | 210 | 202 | 212 | 306 |
| 821 | 403 | 12 | 207 | 201 | 201 | 303 |
| 822 | 407 | 116 | 205 | 202 | 212 | 301 |
| 823 | 406 | 77 | 204 | 212 | 212 | 301 |
| 824 | 403 | 88 | 206 | 212 | 212 | 301 |
| 825 | 401 | 119 | 212 | 212 | 212 | 302 |
| 826 | 401 | 101 | 211 | 201 | 201 | 306 |
| 827 | 405 | 1 | 201 | 212 | 212 | 308 |
| 828 | 406 | 110 | 207 | 201 | 201 | 302 |
| 829 | 401 | 17 | 207 | 201 | 201 | 308 |
| 830 | 405 | 86 | 205 | 201 | 201 | 303 |
| 831 | 401 | 27 | 212 | 202 | 212 | 306 |
| 832 | 407 | 22 | 211 | 201 | 201 | 308 |
| 833 | 401 | 96 | 201 | 201 | 201 | 308 |
| 834 | 405 | 101 | 207 | 212 | 212 | 301 |
| 835 | 401 | 81 | 210 | 212 | 212 | 301 |
| 836 | 406 | 91 | 206 | 212 | 212 | 307 |
| 837 | 406 | 21 | 212 | 202 | 212 | 301 |
| 838 | 401 | 6 | 210 | 202 | 212 | 306 |
| 839 | 406 | 56 | 209 | 202 | 212 | 304 |
| 840 | 406 | 109 | 207 | 201 | 201 | 304 |
| 841 | 401 | 101 | 212 | 202 | 212 | 307 |
| 842 | 407 | 99 | 203 | 202 | 212 | 307 |
| 843 | 401 | 63 | 210 | 202 | 212 | 308 |
| 844 | 401 | 105 | 212 | 202 | 212 | 306 |
| 845 | 404 | 57 | 202 | 212 | 212 | 308 |
| 846 | 401 | 101 | 205 | 202 | 212 | 302 |
| 847 | 401 | 24 | 204 | 201 | 201 | 301 |
| 848 | 402 | 18 | 202 | 202 | 212 | 306 |
| 849 | 401 | 94 | 201 | 212 | 212 | 304 |
| 850 | 404 | 25 | 204 | 202 | 212 | 306 |
| 851 | 403 | 28 | 212 | 202 | 212 | 302 |
| 852 | 404 | 58 | 202 | 212 | 212 | 306 |
| 853 | 401 | 86 | 204 | 212 | 212 | 304 |
| 854 | 405 | 114 | 212 | 202 | 212 | 304 |
| 855 | 407 | 90 | 205 | 212 | 212 | 305 |
| 856 | 402 | 112 | 212 | 201 | 201 | 304 |
| 857 | 406 | 42 | 205 | 212 | 212 | 308 |
| 858 | 401 | 2 | 201 | 201 | 201 | 308 |
| 859 | 404 | 82 | 209 | 202 | 212 | 302 |
| 860 | 401 | 59 | 206 | 201 | 201 | 306 |
| 861 | 405 | 113 | 210 | 201 | 201 | 307 |
| 862 | 402 | 83 | 211 | 212 | 212 | 303 |
| 863 | 403 | 37 | 204 | 202 | 212 | 306 |
| 864 | 401 | 3 | 203 | 202 | 212 | 302 |
| 865 | 407 | 86 | 201 | 212 | 212 | 302 |
| 866 | 402 | 55 | 204 | 201 | 201 | 302 |
| 867 | 405 | 86 | 201 | 202 | 212 | 301 |
| 868 | 405 | 126 | 212 | 202 | 212 | 307 |
| 869 | 406 | 109 | 212 | 201 | 201 | 305 |
| 870 | 403 | 80 | 204 | 212 | 212 | 306 |
| 871 | 401 | 8 | 204 | 212 | 212 | 301 |
| 872 | 405 | 124 | 203 | 201 | 201 | 308 |
| 873 | 407 | 91 | 210 | 201 | 201 | 308 |
| 874 | 407 | 85 | 204 | 212 | 212 | 308 |
| 875 | 405 | 87 | 206 | 202 | 212 | 302 |
| 876 | 403 | 96 | 212 | 201 | 201 | 301 |
| 877 | 402 | 88 | 210 | 202 | 212 | 306 |
| 878 | 406 | 72 | 204 | 202 | 212 | 305 |
| 879 | 404 | 39 | 204 | 212 | 212 | 302 |
| 880 | 401 | 88 | 211 | 201 | 201 | 306 |
| 881 | 401 | 104 | 210 | 202 | 212 | 307 |
| 882 | 403 | 91 | 203 | 201 | 201 | 306 |
| 883 | 401 | 4 | 201 | 201 | 201 | 304 |
| 884 | 401 | 65 | 212 | 212 | 212 | 306 |
| 885 | 404 | 64 | 212 | 201 | 201 | 308 |
| 886 | 401 | 98 | 208 | 201 | 201 | 301 |
| 887 | 401 | 70 | 205 | 201 | 201 | 302 |
| 888 | 401 | 88 | 204 | 202 | 212 | 306 |
| 889 | 401 | 47 | 203 | 202 | 212 | 306 |
| 890 | 401 | 66 | 201 | 212 | 212 | 304 |
| 891 | 406 | 44 | 202 | 201 | 201 | 307 |
| 892 | 401 | 123 | 201 | 201 | 201 | 307 |
| 893 | 404 | 128 | 212 | 201 | 201 | 303 |
| 894 | 401 | 53 | 204 | 201 | 201 | 303 |
| 895 | 401 | 127 | 210 | 201 | 201 | 301 |
| 896 | 407 | 33 | 203 | 202 | 212 | 302 |
| 897 | 404 | 51 | 211 | 201 | 201 | 307 |
| 898 | 401 | 101 | 203 | 201 | 201 | 301 |
| 899 | 403 | 88 | 206 | 202 | 212 | 303 |
| 900 | 402 | 88 | 209 | 212 | 212 | 307 |
| 901 | 401 | 109 | 204 | 212 | 212 | 303 |
| 902 | 401 | 98 | 212 | 201 | 201 | 307 |
| 903 | 401 | 60 | 202 | 201 | 201 | 301 |
| 904 | 404 | 98 | 204 | 212 | 212 | 301 |
| 905 | 407 | 11 | 212 | 212 | 212 | 306 |
| 906 | 403 | 109 | 204 | 201 | 201 | 303 |
| 907 | 407 | 86 | 210 | 212 | 212 | 303 |
| 908 | 401 | 84 | 203 | 212 | 212 | 303 |
| 909 | 401 | 96 | 201 | 201 | 201 | 304 |
| 910 | 406 | 101 | 201 | 212 | 212 | 308 |
| 911 | 401 | 109 | 201 | 212 | 212 | 301 |
| 912 | 407 | 5 | 205 | 201 | 201 | 302 |
| 913 | 404 | 41 | 202 | 202 | 212 | 302 |
| 914 | 401 | 91 | 209 | 212 | 212 | 302 |
| 915 | 401 | 19 | 202 | 201 | 201 | 308 |
| 916 | 406 | 9 | 212 | 212 | 212 | 305 |
| 917 | 402 | 103 | 205 | 201 | 201 | 301 |
| 918 | 401 | 85 | 204 | 212 | 212 | 301 |
| 919 | 406 | 36 | 212 | 201 | 201 | 301 |
| 920 | 401 | 73 | 210 | 202 | 212 | 308 |
| 921 | 406 | 86 | 212 | 212 | 212 | 301 |
| 922 | 407 | 117 | 211 | 202 | 212 | 302 |
| 923 | 403 | 85 | 210 | 202 | 212 | 303 |
| 924 | 403 | 107 | 204 | 201 | 201 | 302 |
| 925 | 401 | 26 | 209 | 212 | 212 | 303 |
| 926 | 402 | 75 | 204 | 202 | 212 | 302 |
| 927 | 401 | 91 | 201 | 201 | 201 | 305 |
| 928 | 403 | 122 | 201 | 201 | 201 | 308 |
| 929 | 401 | 54 | 212 | 201 | 201 | 302 |
| 930 | 401 | 121 | 209 | 202 | 212 | 308 |
| 931 | 407 | 23 | 205 | 202 | 212 | 301 |

Compounds having the formula:

TABLE 6

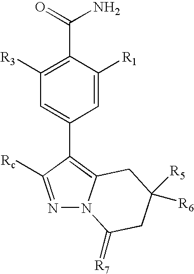

wherein R₁, R₃, R_{C}, R₅, R₆, and R₇ are defined in Table 6:

| Compound | | | | | | |
|---|---|---|---|---|---|---|
| No. | R₁ | R₃ | R_{C} | R₅ | R₆ | R₇ |
| 932 | 402 | 96 | 209 | 201 | 201 | 302 |
| 933 | 407 | 109 | 204 | 201 | 201 | 303 |
| 934 | 404 | 5 | 205 | 201 | 201 | 302 |
| 935 | 401 | 48 | 212 | 202 | 212 | 303 |
| 936 | 404 | 56 | 209 | 202 | 212 | 304 |
| 937 | 402 | 87 | 206 | 202 | 212 | 302 |
| 938 | 402 | 73 | 210 | 202 | 212 | 308 |
| 939 | 401 | 71 | 211 | 202 | 212 | 306 |
| 940 | 401 | 96 | 204 | 201 | 201 | 308 |
| 941 | 405 | 88 | 204 | 202 | 212 | 306 |
| 942 | 404 | 26 | 209 | 212 | 212 | 303 |
| 943 | 406 | 109 | 212 | 201 | 201 | 305 |
| 944 | 401 | 72 | 204 | 202 | 212 | 305 |
| 945 | 401 | 24 | 204 | 201 | 201 | 301 |
| 946 | 402 | 101 | 211 | 201 | 201 | 306 |
| 947 | 401 | 49 | 209 | 201 | 201 | 307 |
| 948 | 404 | 86 | 205 | 201 | 201 | 303 |
| 949 | 402 | 11 | 212 | 212 | 212 | 306 |
| 950 | 405 | 46 | 204 | 202 | 212 | 308 |
| 951 | 402 | 74 | 210 | 201 | 201 | 303 |
| 952 | 401 | 97 | 210 | 201 | 201 | 304 |
| 953 | 405 | 86 | 201 | 202 | 212 | 301 |
| 954 | 404 | 98 | 212 | 201 | 201 | 307 |
| 955 | 401 | 85 | 204 | 212 | 212 | 308 |
| 956 | 405 | 45 | 205 | 201 | 201 | 303 |
| 957 | 406 | 88 | 210 | 202 | 212 | 306 |
| 958 | 407 | 62 | 205 | 212 | 212 | 303 |
| 959 | 401 | 10 | 211 | 202 | 212 | 308 |
| 960 | 401 | 108 | 205 | 212 | 212 | 302 |
| 961 | 403 | 96 | 211 | 201 | 201 | 301 |
| 962 | 401 | 96 | 201 | 201 | 201 | 308 |
| 963 | 401 | 127 | 210 | 201 | 201 | 301 |
| 964 | 401 | 109 | 207 | 201 | 201 | 302 |
| 965 | 401 | 21 | 212 | 202 | 212 | 301 |
| 966 | 401 | 33 | 203 | 202 | 212 | 302 |
| 967 | 401 | 6 | 210 | 202 | 212 | 306 |
| 968 | 401 | 85 | 211 | 212 | 212 | 301 |
| 969 | 401 | 102 | 201 | 201 | 201 | 308 |
| 970 | 405 | 59 | 206 | 201 | 201 | 306 |
| 971 | 401 | 4 | 201 | 201 | 201 | 304 |
| 972 | 404 | 114 | 212 | 202 | 212 | 304 |
| 973 | 402 | 124 | 203 | 201 | 201 | 308 |
| 974 | 401 | 43 | 212 | 202 | 212 | 308 |
| 975 | 405 | 55 | 204 | 201 | 201 | 302 |
| 976 | 401 | 37 | 204 | 202 | 212 | 306 |
| 977 | 403 | 98 | 205 | 201 | 201 | 302 |
| 978 | 401 | 91 | 209 | 212 | 212 | 302 |
| 979 | 401 | 112 | 212 | 201 | 201 | 304 |
| 980 | 406 | 39 | 204 | 212 | 212 | 302 |
| 981 | 401 | 19 | 202 | 201 | 201 | 308 |
| 982 | 403 | 57 | 202 | 212 | 212 | 308 |
| 983 | 405 | 119 | 212 | 212 | 212 | 302 |
| 984 | 404 | 40 | 210 | 202 | 212 | 306 |
| 985 | 407 | 86 | 210 | 212 | 212 | 303 |
| 986 | 403 | 64 | 212 | 201 | 201 | 308 |
| 987 | 402 | 94 | 201 | 212 | 212 | 304 |
| 988 | 407 | 76 | 202 | 202 | 212 | 306 |
| 989 | 401 | 104 | 210 | 202 | 212 | 307 |
| 990 | 403 | 109 | 207 | 201 | 201 | 306 |
| 991 | 405 | 17 | 207 | 201 | 201 | 308 |
| 992 | 401 | 12 | 207 | 201 | 201 | 303 |
| 993 | 401 | 98 | 208 | 201 | 201 | 308 |
| 994 | 402 | 90 | 205 | 212 | 212 | 305 |
| 995 | 402 | 78 | 204 | 201 | 201 | 307 |
| 996 | 403 | 25 | 204 | 202 | 212 | 306 |
| 997 | 407 | 82 | 209 | 202 | 212 | 302 |
| 998 | 406 | 75 | 204 | 202 | 212 | 302 |
| 999 | 403 | 91 | 201 | 201 | 201 | 305 |
| 1000 | 401 | 69 | 210 | 201 | 201 | 303 |
| 1001 | 401 | 9 | 212 | 212 | 212 | 305 |
| 1002 | 405 | 79 | 206 | 212 | 212 | 301 |
| 1003 | 406 | 15 | 211 | 212 | 212 | 302 |
| 1004 | 401 | 98 | 209 | 201 | 201 | 307 |
| 1005 | 405 | 123 | 201 | 201 | 201 | 307 |
| 1006 | 401 | 27 | 212 | 202 | 212 | 306 |
| 1007 | 407 | 109 | 201 | 212 | 212 | 301 |
| 1008 | 401 | 50 | 212 | 202 | 212 | 307 |
| 1009 | 405 | 54 | 212 | 201 | 201 | 302 |
| 1010 | 401 | 117 | 211 | 202 | 212 | 302 |
| 1011 | 406 | 51 | 211 | 201 | 201 | 307 |
| 1012 | 406 | 83 | 211 | 212 | 212 | 303 |
| 1013 | 401 | 120 | 201 | 201 | 201 | 304 |
| 1014 | 406 | 128 | 212 | 201 | 201 | 303 |
| 1015 | 406 | 31 | 208 | 212 | 212 | 305 |
| 1016 | 401 | 13 | 204 | 201 | 201 | 302 |
| 1017 | 407 | 118 | 203 | 212 | 212 | 306 |
| 1018 | 401 | 86 | 212 | 212 | 212 | 301 |
| 1019 | 401 | 86 | 204 | 212 | 212 | 304 |
| 1020 | 404 | 101 | 207 | 212 | 212 | 301 |
| 1021 | 401 | 85 | 210 | 202 | 212 | 303 |
| 1022 | 401 | 42 | 205 | 212 | 212 | 308 |
| 1023 | 402 | 113 | 210 | 201 | 201 | 307 |
| 1024 | 401 | 14 | 203 | 201 | 201 | 308 |
| 1025 | 404 | 36 | 212 | 201 | 201 | 301 |
| 1026 | 403 | 52 | 201 | 202 | 212 | 306 |
| 1027 | 404 | 23 | 205 | 202 | 212 | 301 |
| 1028 | 401 | 2 | 201 | 201 | 201 | 308 |
| 1029 | 405 | 38 | 206 | 201 | 201 | 305 |
| 1030 | 407 | 98 | 208 | 201 | 201 | 301 |
| 1031 | 402 | 100 | 212 | 212 | 212 | 308 |
| 1032 | 406 | 103 | 205 | 201 | 201 | 301 |
| 1033 | 401 | 77 | 204 | 212 | 212 | 301 |
| 1034 | 404 | 3 | 203 | 202 | 212 | 302 |
| 1035 | 401 | 106 | 205 | 212 | 212 | 307 |
| 1036 | 405 | 91 | 212 | 201 | 201 | 304 |
| 1037 | 402 | 86 | 201 | 212 | 212 | 302 |
| 1038 | 403 | 88 | 208 | 212 | 212 | 301 |
| 1039 | 401 | 129 | 211 | 202 | 212 | 302 |
| 1040 | 407 | 47 | 203 | 202 | 212 | 306 |
| 1041 | 402 | 126 | 212 | 202 | 212 | 307 |
| 1042 | 405 | 88 | 210 | 201 | 201 | 301 |
| 1043 | 405 | 116 | 205 | 202 | 212 | 301 |
| 1044 | 406 | 66 | 201 | 212 | 212 | 304 |
| 1045 | 403 | 53 | 204 | 201 | 201 | 303 |
| 1046 | 401 | 91 | 210 | 201 | 201 | 308 |
| 1047 | 405 | 60 | 202 | 201 | 201 | 301 |
| 1048 | 407 | 61 | 202 | 202 | 212 | 301 |
| 1049 | 407 | 65 | 212 | 212 | 212 | 306 |
| 1050 | 405 | 70 | 205 | 201 | 201 | 302 |
| 1051 | 403 | 28 | 212 | 202 | 212 | 302 |
| 1052 | 402 | 99 | 203 | 202 | 212 | 307 |
| 1053 | 406 | 84 | 203 | 212 | 212 | 303 |
| 1054 | 404 | 91 | 206 | 212 | 212 | 307 |
| 1055 | 401 | 88 | 206 | 202 | 212 | 303 |
| 1056 | 401 | 98 | 204 | 212 | 212 | 301 |
| 1057 | 403 | 35 | 209 | 202 | 212 | 301 |
| 1058 | 401 | 107 | 204 | 201 | 201 | 302 |
| 1059 | 401 | 101 | 205 | 202 | 212 | 302 |
| 1060 | 404 | 85 | 204 | 202 | 212 | 301 |
| 1061 | 401 | 121 | 209 | 202 | 212 | 308 |
| 1062 | 401 | 122 | 201 | 201 | 201 | 308 |
| 1063 | 401 | 115 | 206 | 201 | 201 | 308 |
| 1064 | 401 | 63 | 210 | 202 | 212 | 308 |
| 1065 | 401 | 34 | 205 | 201 | 201 | 304 |
| 1066 | 406 | 16 | 205 | 201 | 201 | 306 |
| 1067 | 401 | 18 | 202 | 202 | 212 | 306 |
| 1068 | 404 | 109 | 207 | 201 | 201 | 304 |
| 1069 | 401 | 88 | 206 | 212 | 212 | 301 |
| 1070 | 401 | 58 | 202 | 212 | 212 | 306 |
| 1071 | 407 | 8 | 204 | 212 | 212 | 301 |
| 1072 | 404 | 130 | 206 | 202 | 212 | 306 |
| 1073 | 401 | 96 | 212 | 212 | 212 | 307 |
| 1074 | 403 | 1 | 201 | 212 | 212 | 308 |
| 1075 | 402 | 96 | 212 | 201 | 201 | 301 |
| 1076 | 401 | 96 | 201 | 201 | 201 | 304 |
| 1077 | 401 | 68 | 204 | 212 | 212 | 304 |
| 1078 | 401 | 29 | 210 | 202 | 212 | 306 |
| 1079 | 404 | 110 | 207 | 201 | 201 | 302 |
| 1080 | 407 | 109 | 204 | 212 | 212 | 303 |
| 1081 | 403 | 85 | 212 | 212 | 212 | 307 |
| 1082 | 407 | 88 | 209 | 212 | 212 | 307 |
| 1083 | 401 | 85 | 204 | 212 | 212 | 301 |
| 1084 | 401 | 67 | 211 | 201 | 201 | 302 |
| 1085 | 406 | 81 | 210 | 212 | 212 | 301 |
| 1086 | 401 | 7 | 204 | 201 | 201 | 307 |
| 1087 | 407 | 80 | 204 | 212 | 212 | 306 |
| 1088 | 404 | 93 | 205 | 212 | 212 | 307 |
| 1089 | 401 | 105 | 212 | 202 | 212 | 306 |
| 1090 | 401 | 91 | 203 | 201 | 201 | 306 |
| 1091 | 406 | 30 | 210 | 201 | 201 | 305 |
| 1092 | 402 | 22 | 211 | 201 | 201 | 308 |
| 1093 | 401 | 89 | 206 | 202 | 212 | 306 |
| 1094 | 406 | 96 | 210 | 212 | 212 | 305 |
| 1095 | 401 | 92 | 204 | 212 | 212 | 301 |
| 1096 | 406 | 95 | 204 | 201 | 201 | 308 |
| 1097 | 407 | 101 | 212 | 202 | 212 | 307 |
| 1098 | 403 | 41 | 202 | 202 | 212 | 302 |
| 1099 | 403 | 20 | 201 | 201 | 201 | 305 |
| 1100 | 401 | 125 | 201 | 212 | 212 | 308 |
| 1101 | 402 | 32 | 211 | 212 | 212 | 301 |
| 1102 | 401 | 101 | 201 | 212 | 212 | 308 |
| 1103 | 403 | 88 | 211 | 201 | 201 | 306 |
| 1104 | 401 | 101 | 203 | 201 | 201 | 301 |
| 1105 | 401 | 44 | 202 | 201 | 201 | 307 |
| 1106 | 407 | 111 | 204 | 202 | 212 | 308 |

Compounds having the formula:

TABLE 7

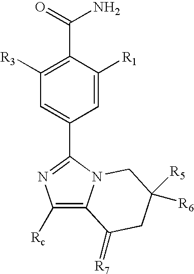

wherein R₁, R₃, R_{C}, R₅, R₆, and R₇ are defined in Table 7:

| Compound | | | | | | |
|---|---|---|---|---|---|---|
| No. | R₁ | R₃ | R_{C} | R₅ | R₆ | R₇ |
| 1107 | 402 | 14 | 203 | 201 | 201 | 308 |
| 1108 | 407 | 17 | 207 | 201 | 201 | 308 |
| 1109 | 404 | 33 | 203 | 202 | 212 | 302 |
| 1110 | 401 | 106 | 205 | 212 | 212 | 307 |
| 1111 | 404 | 88 | 211 | 201 | 201 | 306 |
| 1112 | 402 | 91 | 209 | 212 | 212 | 302 |
| 1113 | 402 | 75 | 204 | 202 | 212 | 302 |
| 1114 | 401 | 96 | 211 | 201 | 201 | 301 |
| 1115 | 401 | 92 | 204 | 212 | 212 | 301 |
| 1116 | 405 | 39 | 204 | 212 | 212 | 302 |
| 1117 | 404 | 112 | 212 | 201 | 201 | 304 |
| 1118 | 406 | 97 | 210 | 201 | 201 | 304 |
| 1119 | 401 | 85 | 212 | 212 | 212 | 307 |
| 1120 | 401 | 98 | 208 | 201 | 201 | 301 |
| 1121 | 402 | 120 | 201 | 201 | 201 | 304 |
| 1122 | 401 | 108 | 205 | 212 | 212 | 302 |
| 1123 | 404 | 88 | 208 | 212 | 212 | 301 |
| 1124 | 402 | 96 | 201 | 201 | 201 | 304 |
| 1125 | 405 | 107 | 204 | 201 | 201 | 302 |
| 1126 | 402 | 128 | 212 | 201 | 201 | 303 |
| 1127 | 401 | 111 | 204 | 202 | 212 | 308 |
| 1128 | 405 | 34 | 205 | 201 | 201 | 304 |
| 1129 | 404 | 86 | 201 | 202 | 212 | 301 |
| 1130 | 401 | 85 | 204 | 202 | 212 | 301 |
| 1131 | 405 | 94 | 201 | 212 | 212 | 304 |
| 1132 | 406 | 86 | 212 | 212 | 212 | 301 |
| 1133 | 407 | 68 | 204 | 212 | 212 | 304 |
| 1134 | 401 | 58 | 202 | 212 | 212 | 306 |
| 1135 | 401 | 54 | 212 | 201 | 201 | 302 |
| 1136 | 403 | 109 | 201 | 212 | 212 | 301 |
| 1137 | 401 | 9 | 212 | 212 | 212 | 305 |
| 1138 | 401 | 66 | 201 | 212 | 212 | 304 |
| 1139 | 401 | 96 | 201 | 201 | 201 | 308 |
| 1140 | 401 | 86 | 201 | 212 | 212 | 302 |
| 1141 | 401 | 101 | 201 | 212 | 212 | 308 |
| 1142 | 401 | 84 | 203 | 212 | 212 | 303 |
| 1143 | 401 | 96 | 212 | 201 | 201 | 301 |
| 1144 | 401 | 109 | 207 | 201 | 201 | 302 |
| 1145 | 405 | 85 | 210 | 202 | 212 | 303 |
| 1146 | 401 | 20 | 201 | 201 | 201 | 305 |
| 1147 | 404 | 57 | 202 | 212 | 212 | 308 |
| 1148 | 402 | 60 | 202 | 201 | 201 | 301 |
| 1149 | 401 | 113 | 210 | 201 | 201 | 307 |
| 1150 | 405 | 109 | 207 | 201 | 201 | 304 |
| 1151 | 401 | 22 | 211 | 201 | 201 | 308 |
| 1152 | 403 | 89 | 206 | 202 | 212 | 306 |
| 1153 | 401 | 101 | 212 | 202 | 212 | 307 |
| 1154 | 401 | 109 | 207 | 201 | 201 | 306 |
| 1155 | 406 | 91 | 210 | 201 | 201 | 308 |
| 1156 | 401 | 91 | 212 | 201 | 201 | 304 |
| 1157 | 403 | 37 | 204 | 202 | 212 | 306 |
| 1158 | 405 | 114 | 212 | 202 | 212 | 304 |
| 1159 | 404 | 80 | 204 | 212 | 212 | 306 |
| 1160 | 407 | 98 | 212 | 201 | 201 | 307 |
| 1161 | 403 | 46 | 204 | 202 | 212 | 308 |
| 1162 | 402 | 98 | 209 | 201 | 201 | 307 |
| 1163 | 407 | 73 | 210 | 202 | 212 | 308 |
| 1164 | 401 | 28 | 212 | 202 | 212 | 302 |
| 1165 | 403 | 129 | 211 | 202 | 212 | 302 |
| 1166 | 405 | 19 | 202 | 201 | 201 | 308 |
| 1167 | 401 | 79 | 206 | 212 | 212 | 301 |
| 1168 | 401 | 83 | 211 | 212 | 212 | 303 |
| 1169 | 402 | 103 | 205 | 201 | 201 | 301 |
| 1170 | 402 | 86 | 210 | 212 | 212 | 303 |
| 1171 | 403 | 64 | 212 | 201 | 201 | 308 |
| 1172 | 407 | 65 | 212 | 212 | 212 | 306 |
| 1173 | 406 | 96 | 210 | 212 | 212 | 305 |
| 1174 | 403 | 8 | 204 | 212 | 212 | 301 |
| 1175 | 401 | 38 | 206 | 201 | 201 | 305 |
| 1176 | 401 | 72 | 204 | 202 | 212 | 305 |
| 1177 | 405 | 59 | 206 | 201 | 201 | 306 |
| 1178 | 406 | 29 | 210 | 202 | 212 | 306 |
| 1179 | 401 | 130 | 206 | 202 | 212 | 306 |
| 1180 | 405 | 104 | 210 | 202 | 212 | 307 |
| 1181 | 401 | 25 | 204 | 202 | 212 | 306 |
| 1182 | 407 | 98 | 204 | 212 | 212 | 301 |
| 1183 | 401 | 115 | 206 | 201 | 201 | 308 |
| 1184 | 405 | 44 | 202 | 201 | 201 | 307 |
| 1185 | 401 | 55 | 204 | 201 | 201 | 302 |
| 1186 | 406 | 81 | 210 | 212 | 212 | 301 |
| 1187 | 406 | 47 | 203 | 202 | 212 | 306 |
| 1188 | 401 | 88 | 204 | 202 | 212 | 306 |
| 1189 | 406 | 88 | 206 | 202 | 212 | 303 |
| 1190 | 406 | 96 | 204 | 201 | 201 | 308 |
| 1191 | 401 | 116 | 205 | 202 | 212 | 301 |
| 1192 | 407 | 90 | 205 | 212 | 212 | 305 |
| 1193 | 401 | 24 | 204 | 201 | 201 | 301 |
| 1194 | 401 | 76 | 202 | 202 | 212 | 306 |
| 1195 | 404 | 121 | 209 | 202 | 212 | 308 |
| 1196 | 401 | 93 | 205 | 212 | 212 | 307 |
| 1197 | 401 | 101 | 203 | 201 | 201 | 301 |
| 1198 | 402 | 91 | 203 | 201 | 201 | 306 |
| 1199 | 401 | 101 | 205 | 202 | 212 | 302 |
| 1200 | 404 | 32 | 211 | 212 | 212 | 301 |
| 1201 | 403 | 6 | 210 | 202 | 212 | 306 |
| 1202 | 404 | 109 | 204 | 201 | 201 | 303 |
| 1203 | 401 | 12 | 207 | 201 | 201 | 303 |
| 1204 | 405 | 101 | 207 | 212 | 212 | 301 |
| 1205 | 407 | 86 | 205 | 201 | 201 | 303 |
| 1206 | 402 | 18 | 202 | 202 | 212 | 306 |
| 1207 | 406 | 96 | 212 | 212 | 212 | 307 |
| 1208 | 401 | 82 | 209 | 202 | 212 | 302 |
| 1209 | 404 | 85 | 204 | 212 | 212 | 308 |
| 1210 | 401 | 127 | 210 | 201 | 201 | 301 |
| 1211 | 405 | 88 | 206 | 212 | 212 | 301 |
| 1212 | 402 | 63 | 210 | 202 | 212 | 308 |
| 1213 | 403 | 16 | 205 | 201 | 201 | 306 |
| 1214 | 401 | 11 | 212 | 212 | 212 | 306 |
| 1215 | 407 | 99 | 203 | 202 | 212 | 307 |
| 1216 | 402 | 31 | 208 | 212 | 212 | 305 |
| 1217 | 405 | 98 | 205 | 201 | 201 | 302 |
| 1218 | 405 | 3 | 203 | 202 | 212 | 302 |
| 1219 | 406 | 10 | 211 | 202 | 212 | 308 |
| 1220 | 403 | 87 | 206 | 202 | 212 | 302 |
| 1221 | 401 | 35 | 209 | 202 | 212 | 301 |
| 1222 | 405 | 26 | 209 | 212 | 212 | 303 |
| 1223 | 407 | 74 | 210 | 201 | 201 | 303 |
| 1224 | 407 | 36 | 212 | 201 | 201 | 301 |
| 1225 | 405 | 56 | 209 | 202 | 212 | 304 |
| 1226 | 403 | 88 | 209 | 212 | 212 | 307 |
| 1227 | 402 | 95 | 204 | 201 | 201 | 308 |
| 1228 | 406 | 96 | 209 | 201 | 201 | 302 |
| 1229 | 404 | 61 | 202 | 202 | 212 | 301 |
| 1230 | 401 | 77 | 204 | 212 | 212 | 301 |
| 1231 | 401 | 27 | 212 | 202 | 212 | 306 |
| 1232 | 403 | 105 | 212 | 202 | 212 | 306 |
| 1233 | 401 | 69 | 210 | 201 | 201 | 303 |
| 1234 | 401 | 41 | 202 | 202 | 212 | 302 |
| 1235 | 404 | 4 | 201 | 201 | 201 | 304 |
| 1236 | 401 | 7 | 204 | 201 | 201 | 307 |
| 1237 | 401 | 86 | 204 | 212 | 212 | 304 |
| 1238 | 401 | 23 | 205 | 202 | 212 | 301 |
| 1239 | 401 | 118 | 203 | 212 | 212 | 306 |
| 1240 | 401 | 101 | 211 | 201 | 201 | 306 |
| 1241 | 406 | 62 | 205 | 212 | 212 | 303 |
| 1242 | 401 | 78 | 204 | 201 | 201 | 307 |
| 1243 | 404 | 1 | 201 | 212 | 212 | 308 |
| 1244 | 401 | 70 | 205 | 201 | 201 | 302 |
| 1245 | 401 | 50 | 212 | 202 | 212 | 307 |
| 1246 | 407 | 91 | 206 | 212 | 212 | 307 |
| 1247 | 404 | 51 | 211 | 201 | 201 | 307 |
| 1248 | 401 | 125 | 201 | 212 | 212 | 308 |
| 1249 | 403 | 102 | 201 | 201 | 201 | 308 |
| 1250 | 402 | 45 | 205 | 201 | 201 | 303 |
| 1251 | 401 | 88 | 210 | 202 | 212 | 306 |
| 1252 | 401 | 88 | 210 | 201 | 201 | 301 |
| 1253 | 401 | 85 | 211 | 212 | 212 | 301 |
| 1254 | 404 | 15 | 211 | 212 | 212 | 302 |
| 1255 | 407 | 67 | 211 | 201 | 201 | 302 |
| 1256 | 403 | 40 | 210 | 202 | 212 | 306 |
| 1257 | 407 | 30 | 210 | 201 | 201 | 305 |
| 1258 | 401 | 53 | 204 | 201 | 201 | 303 |
| 1259 | 401 | 48 | 212 | 202 | 212 | 303 |
| 1260 | 406 | 122 | 201 | 201 | 201 | 308 |
| 1261 | 401 | 123 | 201 | 201 | 201 | 307 |
| 1262 | 407 | 43 | 212 | 202 | 212 | 308 |
| 1263 | 404 | 109 | 212 | 201 | 201 | 305 |
| 1264 | 401 | 13 | 204 | 201 | 201 | 302 |
| 1265 | 401 | 117 | 211 | 202 | 212 | 302 |
| 1266 | 406 | 5 | 205 | 201 | 201 | 302 |
| 1267 | 402 | 124 | 203 | 201 | 201 | 308 |
| 1268 | 401 | 98 | 208 | 201 | 201 | 308 |
| 1269 | 406 | 109 | 204 | 212 | 212 | 303 |
| 1270 | 401 | 52 | 201 | 202 | 212 | 306 |
| 1271 | 406 | 42 | 205 | 212 | 212 | 308 |
| 1272 | 407 | 126 | 212 | 202 | 212 | 307 |
| 1273 | 403 | 119 | 212 | 212 | 212 | 302 |
| 1274 | 403 | 49 | 209 | 201 | 201 | 307 |
| 1275 | 401 | 100 | 212 | 212 | 212 | 308 |
| 1276 | 402 | 21 | 212 | 202 | 212 | 301 |
| 1277 | 401 | 91 | 201 | 201 | 201 | 305 |
| 1278 | 403 | 2 | 201 | 201 | 201 | 308 |
| 1279 | 401 | 71 | 211 | 202 | 212 | 306 |
| 1280 | 401 | 110 | 207 | 201 | 201 | 302 |
| 1281 | 407 | 85 | 204 | 212 | 212 | 301 |

Compounds having the formula:

TABLE 8

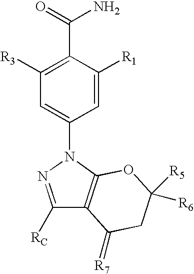

wherein R₁, R₃, R_{C}, R₅, R₆, and R₇ are defined in Table 8:

| Compound | | | | | | |
|---|---|---|---|---|---|---|
| No. | R₁ | R₃ | R_{C} | R₅ | R₆ | R₇ |
| 1282 | 401 | 17 | 207 | 201 | 201 | 308 |
| 1283 | 406 | 33 | 203 | 202 | 212 | 302 |
| 1284 | 401 | 35 | 209 | 202 | 212 | 301 |
| 1285 | 405 | 23 | 205 | 202 | 212 | 301 |
| 1286 | 401 | 76 | 202 | 202 | 212 | 306 |
| 1287 | 401 | 126 | 212 | 202 | 212 | 307 |
| 1288 | 401 | 96 | 201 | 201 | 201 | 308 |
| 1289 | 404 | 28 | 212 | 202 | 212 | 302 |
| 1290 | 407 | 96 | 204 | 201 | 201 | 308 |
| 1291 | 403 | 43 | 212 | 202 | 212 | 308 |
| 1292 | 403 | 11 | 212 | 212 | 212 | 306 |
| 1293 | 401 | 98 | 212 | 201 | 201 | 307 |
| 1294 | 401 | 86 | 210 | 212 | 212 | 303 |
| 1295 | 405 | 40 | 210 | 202 | 212 | 306 |
| 1296 | 402 | 44 | 202 | 201 | 201 | 307 |
| 1297 | 401 | 109 | 204 | 201 | 201 | 303 |
| 1298 | 405 | 121 | 209 | 202 | 212 | 308 |
| 1299 | 401 | 104 | 210 | 202 | 212 | 307 |
| 1300 | 401 | 47 | 203 | 202 | 212 | 306 |
| 1301 | 403 | 109 | 212 | 201 | 201 | 305 |
| 1302 | 401 | 2 | 201 | 201 | 201 | 308 |
| 1303 | 405 | 85 | 204 | 212 | 212 | 301 |
| 1304 | 401 | 115 | 206 | 201 | 201 | 308 |
| 1305 | 404 | 91 | 203 | 201 | 201 | 306 |
| 1306 | 403 | 84 | 203 | 212 | 212 | 303 |
| 1307 | 401 | 24 | 204 | 201 | 201 | 301 |
| 1308 | 406 | 78 | 204 | 201 | 201 | 307 |
| 1309 | 402 | 88 | 204 | 202 | 212 | 306 |
| 1310 | 404 | 96 | 212 | 212 | 212 | 307 |
| 1311 | 401 | 15 | 211 | 212 | 212 | 302 |
| 1312 | 404 | 120 | 201 | 201 | 201 | 304 |
| 1313 | 402 | 34 | 205 | 201 | 201 | 304 |
| 1314 | 405 | 41 | 202 | 202 | 212 | 302 |
| 1315 | 407 | 88 | 210 | 202 | 212 | 306 |
| 1316 | 404 | 70 | 205 | 201 | 201 | 302 |
| 1317 | 404 | 85 | 212 | 212 | 212 | 307 |
| 1318 | 405 | 102 | 201 | 201 | 201 | 308 |
| 1319 | 403 | 4 | 201 | 201 | 201 | 304 |
| 1320 | 404 | 114 | 212 | 202 | 212 | 304 |
| 1321 | 401 | 54 | 212 | 201 | 201 | 302 |
| 1322 | 401 | 68 | 204 | 212 | 212 | 304 |
| 1323 | 405 | 105 | 212 | 202 | 212 | 306 |
| 1324 | 403 | 77 | 204 | 212 | 212 | 301 |
| 1325 | 405 | 16 | 205 | 201 | 201 | 306 |
| 1326 | 401 | 80 | 204 | 212 | 212 | 306 |
| 1327 | 402 | 109 | 207 | 201 | 201 | 304 |
| 1328 | 404 | 109 | 207 | 201 | 201 | 306 |
| 1329 | 401 | 74 | 210 | 201 | 201 | 303 |
| 1330 | 402 | 86 | 204 | 212 | 212 | 304 |
| 1331 | 401 | 86 | 205 | 201 | 201 | 303 |
| 1332 | 403 | 57 | 202 | 212 | 212 | 308 |
| 1333 | 401 | 39 | 204 | 212 | 212 | 302 |
| 1334 | 401 | 93 | 205 | 212 | 212 | 307 |
| 1335 | 401 | 81 | 210 | 212 | 212 | 301 |
| 1336 | 401 | 112 | 212 | 201 | 201 | 304 |
| 1337 | 405 | 129 | 211 | 202 | 212 | 302 |
| 1338 | 401 | 50 | 212 | 202 | 212 | 307 |
| 1339 | 403 | 117 | 211 | 202 | 212 | 302 |
| 1340 | 404 | 52 | 201 | 202 | 212 | 306 |
| 1341 | 402 | 67 | 211 | 201 | 201 | 302 |
| 1342 | 401 | 88 | 210 | 201 | 201 | 301 |
| 1343 | 405 | 96 | 210 | 212 | 212 | 305 |
| 1344 | 401 | 109 | 204 | 212 | 212 | 303 |
| 1345 | 406 | 92 | 204 | 212 | 212 | 301 |
| 1346 | 401 | 85 | 210 | 202 | 212 | 303 |
| 1347 | 401 | 88 | 211 | 201 | 201 | 306 |
| 1348 | 404 | 42 | 205 | 212 | 212 | 308 |
| 1349 | 405 | 89 | 206 | 202 | 212 | 306 |
| 1350 | 403 | 36 | 212 | 201 | 201 | 301 |
| 1351 | 401 | 6 | 210 | 202 | 212 | 306 |
| 1352 | 407 | 59 | 206 | 201 | 201 | 306 |
| 1353 | 402 | 61 | 202 | 202 | 212 | 301 |
| 1354 | 403 | 96 | 211 | 201 | 201 | 301 |
| 1355 | 404 | 91 | 212 | 201 | 201 | 304 |
| 1356 | 406 | 123 | 201 | 201 | 201 | 307 |
| 1357 | 407 | 5 | 205 | 201 | 201 | 302 |
| 1358 | 403 | 71 | 211 | 202 | 212 | 306 |
| 1359 | 404 | 88 | 206 | 202 | 212 | 303 |
| 1360 | 401 | 128 | 212 | 201 | 201 | 303 |
| 1361 | 401 | 48 | 212 | 202 | 212 | 303 |
| 1362 | 407 | 96 | 209 | 201 | 201 | 302 |
| 1363 | 401 | 12 | 207 | 201 | 201 | 303 |
| 1364 | 401 | 85 | 204 | 202 | 212 | 301 |
| 1365 | 404 | 73 | 210 | 202 | 212 | 308 |
| 1366 | 406 | 101 | 203 | 201 | 201 | 301 |
| 1367 | 407 | 103 | 205 | 201 | 201 | 301 |
| 1368 | 402 | 88 | 209 | 212 | 212 | 307 |
| 1369 | 401 | 66 | 201 | 212 | 212 | 304 |
| 1370 | 405 | 20 | 201 | 201 | 201 | 305 |
| 1371 | 407 | 45 | 205 | 201 | 201 | 303 |
| 1372 | 401 | 113 | 210 | 201 | 201 | 307 |
| 1373 | 405 | 51 | 211 | 201 | 201 | 307 |
| 1374 | 401 | 46 | 204 | 202 | 212 | 308 |
| 1375 | 401 | 10 | 211 | 202 | 212 | 308 |
| 1376 | 401 | 94 | 201 | 212 | 212 | 304 |
| 1377 | 401 | 18 | 202 | 202 | 212 | 306 |
| 1378 | 403 | 22 | 211 | 201 | 201 | 308 |
| 1379 | 406 | 7 | 204 | 201 | 201 | 307 |
| 1380 | 401 | 96 | 212 | 201 | 201 | 301 |
| 1381 | 401 | 124 | 203 | 201 | 201 | 308 |
| 1382 | 402 | 32 | 211 | 212 | 212 | 301 |
| 1383 | 401 | 3 | 203 | 202 | 212 | 302 |
| 1384 | 403 | 55 | 204 | 201 | 201 | 302 |
| 1385 | 401 | 86 | 212 | 212 | 212 | 301 |
| 1386 | 401 | 88 | 208 | 212 | 212 | 301 |
| 1387 | 402 | 101 | 201 | 212 | 212 | 308 |
| 1388 | 406 | 95 | 204 | 201 | 201 | 308 |
| 1389 | 401 | 109 | 207 | 201 | 201 | 302 |
| 1390 | 403 | 30 | 210 | 201 | 201 | 305 |
| 1391 | 402 | 8 | 204 | 212 | 212 | 301 |
| 1392 | 401 | 98 | 205 | 201 | 201 | 302 |
| 1393 | 401 | 56 | 209 | 202 | 212 | 304 |
| 1394 | 404 | 13 | 204 | 201 | 201 | 302 |
| 1395 | 403 | 98 | 208 | 201 | 201 | 308 |
| 1396 | 406 | 79 | 206 | 212 | 212 | 301 |
| 1397 | 406 | 96 | 201 | 201 | 201 | 304 |
| 1398 | 403 | 75 | 204 | 202 | 212 | 302 |
| 1399 | 401 | 108 | 205 | 212 | 212 | 302 |
| 1400 | 401 | 98 | 208 | 201 | 201 | 301 |
| 1401 | 401 | 19 | 202 | 201 | 201 | 308 |
| 1402 | 401 | 91 | 206 | 212 | 212 | 307 |
| 1403 | 403 | 98 | 209 | 201 | 201 | 307 |
| 1404 | 407 | 29 | 210 | 202 | 212 | 306 |
| 1405 | 401 | 85 | 211 | 212 | 212 | 301 |
| 1406 | 405 | 38 | 206 | 201 | 201 | 305 |
| 1407 | 404 | 107 | 204 | 201 | 201 | 302 |
| 1408 | 401 | 119 | 212 | 212 | 212 | 302 |
| 1409 | 402 | 110 | 207 | 201 | 201 | 302 |
| 1410 | 407 | 116 | 205 | 202 | 212 | 301 |
| 1411 | 402 | 87 | 206 | 202 | 212 | 302 |
| 1412 | 402 | 101 | 207 | 212 | 212 | 301 |
| 1413 | 407 | 72 | 204 | 202 | 212 | 305 |
| 1414 | 401 | 60 | 202 | 201 | 201 | 301 |
| 1415 | 401 | 65 | 212 | 212 | 212 | 306 |
| 1416 | 402 | 101 | 205 | 202 | 212 | 302 |
| 1417 | 406 | 53 | 204 | 201 | 201 | 303 |
| 1418 | 407 | 122 | 201 | 201 | 201 | 308 |
| 1419 | 402 | 9 | 212 | 212 | 212 | 305 |
| 1420 | 406 | 101 | 212 | 202 | 212 | 307 |
| 1421 | 401 | 106 | 205 | 212 | 212 | 307 |
| 1422 | 405 | 109 | 201 | 212 | 212 | 301 |
| 1423 | 401 | 21 | 212 | 202 | 212 | 301 |
| 1424 | 401 | 86 | 201 | 202 | 212 | 301 |
| 1425 | 402 | 99 | 203 | 202 | 212 | 307 |
| 1426 | 401 | 127 | 210 | 201 | 201 | 301 |
| 1427 | 401 | 100 | 212 | 212 | 212 | 308 |
| 1428 | 401 | 26 | 209 | 212 | 212 | 303 |
| 1429 | 406 | 63 | 210 | 202 | 212 | 308 |
| 1430 | 401 | 91 | 210 | 201 | 201 | 308 |
| 1431 | 406 | 101 | 211 | 201 | 201 | 306 |
| 1432 | 404 | 130 | 206 | 202 | 212 | 306 |
| 1433 | 407 | 118 | 203 | 212 | 212 | 306 |
| 1434 | 401 | 85 | 204 | 212 | 212 | 308 |
| 1435 | 402 | 27 | 212 | 202 | 212 | 306 |
| 1436 | 406 | 91 | 201 | 201 | 201 | 305 |
| 1437 | 401 | 49 | 209 | 201 | 201 | 307 |
| 1438 | 406 | 64 | 212 | 201 | 201 | 308 |
| 1439 | 405 | 31 | 208 | 212 | 212 | 305 |
| 1440 | 405 | 82 | 209 | 202 | 212 | 302 |
| 1441 | 401 | 14 | 203 | 201 | 201 | 308 |
| 1442 | 401 | 98 | 204 | 212 | 212 | 301 |
| 1443 | 407 | 91 | 209 | 212 | 212 | 302 |
| 1444 | 401 | 86 | 201 | 212 | 212 | 302 |
| 1445 | 407 | 83 | 211 | 212 | 212 | 303 |
| 1446 | 401 | 37 | 204 | 202 | 212 | 306 |
| 1447 | 406 | 1 | 201 | 212 | 212 | 308 |
| 1448 | 401 | 69 | 210 | 201 | 201 | 303 |
| 1449 | 401 | 90 | 205 | 212 | 212 | 305 |
| 1450 | 407 | 111 | 204 | 202 | 212 | 308 |
| 1451 | 401 | 88 | 206 | 212 | 212 | 301 |
| 1452 | 407 | 25 | 204 | 202 | 212 | 306 |
| 1453 | 401 | 97 | 210 | 201 | 201 | 304 |
| 1454 | 404 | 125 | 201 | 212 | 212 | 308 |
| 1455 | 407 | 62 | 205 | 212 | 212 | 303 |
| 1456 | 406 | 58 | 202 | 212 | 212 | 306 |

Compounds having the formula:

TABLE 9

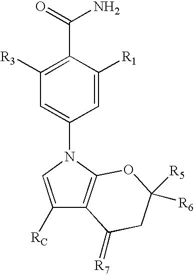

wherein R₁, R₃, R_{C}, R₅, R₆, and R₇ are defined in Table 9:

| Compound | | | | | | |
|---|---|---|---|---|---|---|
| No. | R₁ | R₃ | R_{C} | R₅ | R₆ | R₇ |
| 1457 | 402 | 96 | 209 | 201 | 201 | 302 |
| 1458 | 407 | 109 | 204 | 201 | 201 | 303 |
| 1459 | 404 | 5 | 205 | 201 | 201 | 302 |
| 1460 | 401 | 48 | 212 | 202 | 212 | 303 |
| 1461 | 404 | 56 | 209 | 202 | 212 | 304 |
| 1462 | 402 | 87 | 206 | 202 | 212 | 302 |
| 1463 | 402 | 73 | 210 | 202 | 212 | 308 |
| 1464 | 401 | 71 | 211 | 202 | 212 | 306 |
| 1465 | 401 | 96 | 204 | 201 | 201 | 308 |
| 1466 | 405 | 88 | 204 | 202 | 212 | 306 |
| 1467 | 404 | 26 | 209 | 212 | 212 | 303 |
| 1468 | 406 | 109 | 212 | 201 | 201 | 305 |
| 1469 | 401 | 72 | 204 | 202 | 212 | 305 |
| 1470 | 401 | 24 | 204 | 201 | 201 | 301 |
| 1471 | 402 | 101 | 211 | 201 | 201 | 306 |
| 1472 | 401 | 49 | 209 | 201 | 201 | 307 |
| 1473 | 404 | 86 | 205 | 201 | 201 | 303 |
| 1474 | 402 | 11 | 212 | 212 | 212 | 306 |
| 1475 | 405 | 46 | 204 | 202 | 212 | 308 |
| 1476 | 402 | 74 | 210 | 201 | 201 | 303 |
| 1477 | 401 | 97 | 210 | 201 | 201 | 304 |
| 1478 | 405 | 86 | 201 | 202 | 212 | 301 |
| 1479 | 404 | 98 | 212 | 201 | 201 | 307 |
| 1480 | 401 | 85 | 204 | 212 | 212 | 308 |
| 1481 | 405 | 45 | 205 | 201 | 201 | 303 |
| 1482 | 406 | 88 | 210 | 202 | 212 | 306 |
| 1483 | 407 | 62 | 205 | 212 | 212 | 303 |
| 1484 | 401 | 10 | 211 | 202 | 212 | 308 |
| 1485 | 401 | 108 | 205 | 212 | 212 | 302 |
| 1486 | 403 | 96 | 211 | 201 | 201 | 301 |
| 1487 | 401 | 96 | 201 | 201 | 201 | 308 |
| 1488 | 401 | 127 | 210 | 201 | 201 | 301 |
| 1489 | 401 | 109 | 207 | 201 | 201 | 302 |
| 1490 | 401 | 21 | 212 | 202 | 212 | 301 |
| 1491 | 401 | 33 | 203 | 202 | 212 | 302 |
| 1492 | 401 | 6 | 210 | 202 | 212 | 306 |
| 1493 | 401 | 85 | 211 | 212 | 212 | 301 |
| 1494 | 401 | 102 | 201 | 201 | 201 | 308 |
| 1495 | 405 | 59 | 206 | 201 | 201 | 306 |
| 1496 | 401 | 4 | 201 | 201 | 201 | 304 |
| 1497 | 404 | 114 | 212 | 202 | 212 | 304 |
| 1498 | 402 | 124 | 203 | 201 | 201 | 308 |
| 1499 | 401 | 43 | 212 | 202 | 212 | 308 |
| 1500 | 405 | 55 | 204 | 201 | 201 | 302 |
| 1501 | 401 | 37 | 204 | 202 | 212 | 306 |
| 1502 | 403 | 98 | 205 | 201 | 201 | 302 |
| 1503 | 401 | 91 | 209 | 212 | 212 | 302 |
| 1504 | 401 | 112 | 212 | 201 | 201 | 304 |
| 1505 | 406 | 39 | 204 | 212 | 212 | 302 |
| 1506 | 401 | 19 | 202 | 201 | 201 | 308 |
| 1507 | 403 | 57 | 202 | 212 | 212 | 308 |
| 1508 | 405 | 119 | 212 | 212 | 212 | 302 |
| 1509 | 404 | 40 | 210 | 202 | 212 | 306 |
| 1510 | 407 | 86 | 210 | 212 | 212 | 303 |
| 1511 | 403 | 64 | 212 | 201 | 201 | 308 |
| 1512 | 402 | 94 | 201 | 212 | 212 | 304 |
| 1513 | 407 | 76 | 202 | 202 | 212 | 306 |
| 1514 | 401 | 104 | 210 | 202 | 212 | 307 |
| 1515 | 403 | 109 | 207 | 201 | 201 | 306 |
| 1516 | 405 | 17 | 207 | 201 | 201 | 308 |
| 1517 | 401 | 12 | 207 | 201 | 201 | 303 |
| 1518 | 401 | 98 | 208 | 201 | 201 | 308 |
| 1519 | 402 | 90 | 205 | 212 | 212 | 305 |
| 1520 | 402 | 78 | 204 | 201 | 201 | 307 |
| 1521 | 403 | 25 | 204 | 202 | 212 | 306 |
| 1522 | 407 | 82 | 209 | 202 | 212 | 302 |
| 1523 | 406 | 75 | 204 | 202 | 212 | 302 |
| 1524 | 403 | 91 | 201 | 201 | 201 | 305 |
| 1525 | 401 | 69 | 210 | 201 | 201 | 303 |
| 1526 | 401 | 9 | 212 | 212 | 212 | 305 |
| 1527 | 405 | 79 | 206 | 212 | 212 | 301 |
| 1528 | 406 | 15 | 211 | 212 | 212 | 302 |
| 1529 | 401 | 98 | 209 | 201 | 201 | 307 |
| 1530 | 405 | 123 | 201 | 201 | 201 | 307 |
| 1531 | 401 | 27 | 212 | 202 | 212 | 306 |
| 1532 | 407 | 109 | 201 | 212 | 212 | 301 |
| 1533 | 401 | 50 | 212 | 202 | 212 | 307 |
| 1534 | 405 | 54 | 212 | 201 | 201 | 302 |
| 1535 | 401 | 117 | 211 | 202 | 212 | 302 |
| 1536 | 406 | 51 | 211 | 201 | 201 | 307 |
| 1537 | 406 | 83 | 211 | 212 | 212 | 303 |
| 1538 | 401 | 120 | 201 | 201 | 201 | 304 |
| 1539 | 406 | 128 | 212 | 201 | 201 | 303 |
| 1540 | 406 | 31 | 208 | 212 | 212 | 305 |
| 1541 | 401 | 13 | 204 | 201 | 201 | 302 |
| 1542 | 407 | 118 | 203 | 212 | 212 | 306 |
| 1543 | 401 | 86 | 212 | 212 | 212 | 301 |
| 1544 | 401 | 86 | 204 | 212 | 212 | 304 |
| 1545 | 404 | 101 | 207 | 212 | 212 | 301 |
| 1546 | 401 | 85 | 210 | 202 | 212 | 303 |
| 1547 | 401 | 42 | 205 | 212 | 212 | 308 |
| 1548 | 402 | 113 | 210 | 201 | 201 | 307 |
| 1549 | 401 | 14 | 203 | 201 | 201 | 308 |
| 1550 | 404 | 36 | 212 | 201 | 201 | 301 |
| 1551 | 403 | 52 | 201 | 202 | 212 | 306 |
| 1552 | 404 | 23 | 205 | 202 | 212 | 301 |
| 1553 | 401 | 2 | 201 | 201 | 201 | 308 |
| 1554 | 405 | 38 | 206 | 201 | 201 | 305 |
| 1555 | 407 | 98 | 208 | 201 | 201 | 301 |
| 1556 | 402 | 100 | 212 | 212 | 212 | 308 |
| 1557 | 406 | 103 | 205 | 201 | 201 | 301 |
| 1558 | 401 | 77 | 204 | 212 | 212 | 301 |
| 1559 | 404 | 3 | 203 | 202 | 212 | 302 |
| 1560 | 401 | 106 | 205 | 212 | 212 | 307 |
| 1561 | 405 | 91 | 212 | 201 | 201 | 304 |
| 1562 | 402 | 86 | 201 | 212 | 212 | 302 |
| 1563 | 403 | 88 | 208 | 212 | 212 | 301 |
| 1564 | 401 | 129 | 211 | 202 | 212 | 302 |
| 1565 | 407 | 47 | 203 | 202 | 212 | 306 |
| 1566 | 402 | 126 | 212 | 202 | 212 | 307 |
| 1567 | 405 | 88 | 210 | 201 | 201 | 301 |
| 1568 | 405 | 116 | 205 | 202 | 212 | 301 |
| 1569 | 406 | 66 | 201 | 212 | 212 | 304 |
| 1570 | 403 | 53 | 204 | 201 | 201 | 303 |
| 1571 | 401 | 91 | 210 | 201 | 201 | 308 |
| 1572 | 405 | 60 | 202 | 201 | 201 | 301 |
| 1573 | 407 | 61 | 202 | 202 | 212 | 301 |
| 1574 | 407 | 65 | 212 | 212 | 212 | 306 |
| 1575 | 405 | 70 | 205 | 201 | 201 | 302 |
| 1576 | 403 | 28 | 212 | 202 | 212 | 302 |
| 1577 | 402 | 99 | 203 | 202 | 212 | 307 |
| 1578 | 406 | 84 | 203 | 212 | 212 | 303 |
| 1579 | 404 | 91 | 206 | 212 | 212 | 307 |
| 1580 | 401 | 88 | 206 | 202 | 212 | 303 |
| 1581 | 401 | 98 | 204 | 212 | 212 | 301 |
| 1582 | 403 | 35 | 209 | 202 | 212 | 301 |
| 1583 | 401 | 107 | 204 | 201 | 201 | 302 |
| 1584 | 401 | 101 | 205 | 202 | 212 | 302 |
| 1585 | 404 | 85 | 204 | 202 | 212 | 301 |
| 1586 | 401 | 121 | 209 | 202 | 212 | 308 |
| 1587 | 401 | 122 | 201 | 201 | 201 | 308 |
| 1588 | 401 | 115 | 206 | 201 | 201 | 308 |
| 1589 | 401 | 63 | 210 | 202 | 212 | 308 |
| 1590 | 401 | 34 | 205 | 201 | 201 | 304 |
| 1591 | 406 | 16 | 205 | 201 | 201 | 306 |
| 1592 | 401 | 18 | 202 | 202 | 212 | 306 |
| 1593 | 404 | 109 | 207 | 201 | 201 | 304 |
| 1594 | 401 | 88 | 206 | 212 | 212 | 301 |
| 1595 | 401 | 58 | 202 | 212 | 212 | 306 |
| 1596 | 407 | 8 | 204 | 212 | 212 | 301 |
| 1597 | 404 | 130 | 206 | 202 | 212 | 306 |
| 1598 | 401 | 96 | 212 | 212 | 212 | 307 |
| 1599 | 403 | 1 | 201 | 212 | 212 | 308 |
| 1600 | 402 | 96 | 212 | 201 | 201 | 301 |
| 1601 | 401 | 96 | 201 | 201 | 201 | 304 |
| 1602 | 401 | 68 | 204 | 212 | 212 | 304 |
| 1603 | 401 | 29 | 210 | 202 | 212 | 306 |
| 1604 | 404 | 110 | 207 | 201 | 201 | 302 |
| 1605 | 407 | 109 | 204 | 212 | 212 | 303 |
| 1606 | 403 | 85 | 212 | 212 | 212 | 307 |
| 1607 | 407 | 88 | 209 | 212 | 212 | 307 |
| 1608 | 401 | 85 | 204 | 212 | 212 | 301 |
| 1609 | 401 | 67 | 211 | 201 | 201 | 302 |
| 1610 | 406 | 81 | 210 | 212 | 212 | 301 |
| 1611 | 401 | 7 | 204 | 201 | 201 | 307 |
| 1612 | 407 | 80 | 204 | 212 | 212 | 306 |
| 1613 | 404 | 93 | 205 | 212 | 212 | 307 |
| 1614 | 401 | 105 | 212 | 202 | 212 | 306 |
| 1615 | 401 | 91 | 203 | 201 | 201 | 306 |
| 1616 | 406 | 30 | 210 | 201 | 201 | 305 |
| 1617 | 402 | 22 | 211 | 201 | 201 | 308 |
| 1618 | 401 | 89 | 206 | 202 | 212 | 306 |
| 1619 | 406 | 96 | 210 | 212 | 212 | 305 |
| 1620 | 401 | 92 | 204 | 212 | 212 | 301 |
| 1621 | 406 | 95 | 204 | 201 | 201 | 308 |
| 1622 | 407 | 101 | 212 | 202 | 212 | 307 |
| 1623 | 403 | 41 | 202 | 202 | 212 | 302 |
| 1624 | 403 | 20 | 201 | 201 | 201 | 305 |
| 1625 | 401 | 125 | 201 | 212 | 212 | 308 |
| 1626 | 402 | 32 | 211 | 212 | 212 | 301 |
| 1627 | 401 | 101 | 201 | 212 | 212 | 308 |
| 1628 | 403 | 88 | 211 | 201 | 201 | 306 |
| 1629 | 401 | 101 | 203 | 201 | 201 | 301 |
| 1630 | 401 | 44 | 202 | 201 | 201 | 307 |
| 1631 | 407 | 111 | 204 | 202 | 212 | 308 |

Compounds having the formula:

TABLE 10

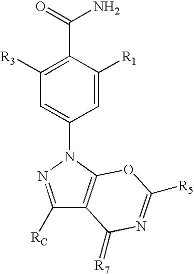

wherein R₁, R₃, R_{C}, R₅, R₆, and R₇ are defined in Table 10:

| Compound | | | | | |
|---|---|---|---|---|---|
| No. | R₁ | R₃ | R_{C} | R₅ | R₇ |
| 1632 | 402 | 14 | 203 | 201 | 308 |
| 1633 | 407 | 17 | 207 | 201 | 308 |
| 1634 | 404 | 33 | 203 | 202 | 302 |
| 1635 | 401 | 106 | 205 | 212 | 307 |
| 1636 | 404 | 88 | 211 | 201 | 306 |
| 1637 | 402 | 91 | 209 | 212 | 302 |
| 1638 | 402 | 75 | 204 | 202 | 302 |
| 1639 | 401 | 96 | 211 | 201 | 301 |
| 1640 | 401 | 92 | 204 | 212 | 301 |
| 1641 | 405 | 39 | 204 | 212 | 302 |
| 1642 | 404 | 112 | 212 | 201 | 304 |
| 1643 | 406 | 97 | 210 | 201 | 304 |
| 1644 | 401 | 85 | 212 | 212 | 307 |
| 1645 | 401 | 98 | 208 | 201 | 301 |
| 1646 | 402 | 120 | 201 | 201 | 304 |
| 1647 | 401 | 108 | 205 | 212 | 302 |
| 1648 | 404 | 88 | 208 | 212 | 301 |
| 1649 | 402 | 96 | 201 | 201 | 304 |
| 1650 | 405 | 107 | 204 | 201 | 302 |
| 1651 | 402 | 128 | 212 | 201 | 303 |
| 1652 | 401 | 111 | 204 | 202 | 308 |
| 1653 | 405 | 34 | 205 | 201 | 304 |
| 1654 | 404 | 86 | 201 | 202 | 301 |
| 1655 | 401 | 85 | 204 | 202 | 301 |
| 1656 | 405 | 94 | 201 | 212 | 304 |
| 1657 | 406 | 86 | 212 | 212 | 301 |
| 1658 | 407 | 68 | 204 | 212 | 304 |
| 1659 | 401 | 58 | 202 | 212 | 306 |
| 1660 | 401 | 54 | 212 | 201 | 302 |
| 1661 | 403 | 109 | 201 | 212 | 301 |
| 1662 | 401 | 9 | 212 | 212 | 305 |
| 1663 | 401 | 66 | 201 | 212 | 304 |
| 1664 | 401 | 96 | 201 | 201 | 308 |
| 1665 | 401 | 86 | 201 | 212 | 302 |
| 1666 | 401 | 101 | 201 | 212 | 308 |
| 1667 | 401 | 84 | 203 | 212 | 303 |
| 1668 | 401 | 96 | 212 | 201 | 301 |
| 1669 | 401 | 109 | 207 | 201 | 302 |
| 1670 | 405 | 85 | 210 | 202 | 303 |
| 1671 | 401 | 20 | 201 | 201 | 305 |
| 1672 | 404 | 57 | 202 | 212 | 308 |
| 1673 | 402 | 60 | 202 | 201 | 301 |
| 1674 | 401 | 113 | 210 | 201 | 307 |
| 1675 | 405 | 109 | 207 | 201 | 304 |
| 1676 | 401 | 22 | 211 | 201 | 308 |
| 1677 | 403 | 89 | 206 | 202 | 306 |
| 1678 | 401 | 101 | 212 | 202 | 307 |
| 1679 | 401 | 109 | 207 | 201 | 306 |
| 1680 | 406 | 91 | 210 | 201 | 308 |
| 1681 | 401 | 91 | 212 | 201 | 304 |
| 1682 | 403 | 37 | 204 | 202 | 306 |
| 1683 | 405 | 114 | 212 | 202 | 304 |
| 1684 | 404 | 80 | 204 | 212 | 306 |
| 1685 | 407 | 98 | 212 | 201 | 307 |
| 1686 | 403 | 46 | 204 | 202 | 308 |
| 1687 | 402 | 98 | 209 | 201 | 307 |
| 1688 | 407 | 73 | 210 | 202 | 308 |
| 1689 | 401 | 28 | 212 | 202 | 302 |
| 1690 | 403 | 129 | 211 | 202 | 302 |
| 1691 | 405 | 19 | 202 | 201 | 308 |
| 1692 | 401 | 79 | 206 | 212 | 301 |
| 1693 | 401 | 83 | 211 | 212 | 303 |
| 1694 | 402 | 103 | 205 | 201 | 301 |
| 1695 | 402 | 86 | 210 | 212 | 303 |
| 1696 | 403 | 64 | 212 | 201 | 308 |
| 1697 | 407 | 65 | 212 | 212 | 306 |
| 1698 | 406 | 96 | 210 | 212 | 305 |
| 1699 | 403 | 8 | 204 | 212 | 301 |
| 1700 | 401 | 38 | 206 | 201 | 305 |
| 1701 | 401 | 72 | 204 | 202 | 305 |
| 1702 | 405 | 59 | 206 | 201 | 306 |
| 1703 | 406 | 29 | 210 | 202 | 306 |
| 1704 | 401 | 130 | 206 | 202 | 306 |
| 1705 | 405 | 104 | 210 | 202 | 307 |
| 1706 | 401 | 25 | 204 | 202 | 306 |
| 1707 | 407 | 98 | 204 | 212 | 301 |
| 1708 | 401 | 115 | 206 | 201 | 308 |
| 1709 | 405 | 44 | 202 | 201 | 307 |
| 1710 | 401 | 55 | 204 | 201 | 302 |
| 1711 | 406 | 81 | 210 | 212 | 301 |
| 1712 | 406 | 47 | 203 | 202 | 306 |
| 1713 | 401 | 88 | 204 | 202 | 306 |
| 1714 | 406 | 88 | 206 | 202 | 303 |
| 1715 | 406 | 96 | 204 | 201 | 308 |
| 1716 | 401 | 116 | 205 | 202 | 301 |
| 1717 | 407 | 90 | 205 | 212 | 305 |
| 1718 | 401 | 24 | 204 | 201 | 301 |
| 1719 | 401 | 76 | 202 | 202 | 306 |
| 1720 | 404 | 121 | 209 | 202 | 308 |
| 1721 | 401 | 93 | 205 | 212 | 307 |
| 1722 | 401 | 101 | 203 | 201 | 301 |
| 1723 | 402 | 91 | 203 | 201 | 306 |
| 1724 | 401 | 101 | 205 | 202 | 302 |
| 1725 | 404 | 32 | 211 | 212 | 301 |
| 1726 | 403 | 6 | 210 | 202 | 306 |
| 1727 | 404 | 109 | 204 | 201 | 303 |
| 1728 | 401 | 12 | 207 | 201 | 303 |
| 1729 | 405 | 101 | 207 | 212 | 301 |
| 1730 | 407 | 86 | 205 | 201 | 303 |
| 1731 | 402 | 18 | 202 | 202 | 306 |
| 1732 | 406 | 96 | 212 | 212 | 307 |
| 1733 | 401 | 82 | 209 | 202 | 302 |
| 1734 | 404 | 85 | 204 | 212 | 308 |
| 1735 | 401 | 127 | 210 | 201 | 301 |
| 1736 | 405 | 88 | 206 | 212 | 301 |
| 1737 | 402 | 63 | 210 | 202 | 308 |
| 1738 | 403 | 16 | 205 | 201 | 306 |
| 1739 | 401 | 11 | 212 | 212 | 306 |
| 1740 | 407 | 99 | 203 | 202 | 307 |
| 1741 | 402 | 31 | 208 | 212 | 305 |
| 1742 | 405 | 98 | 205 | 201 | 302 |
| 1743 | 405 | 3 | 203 | 202 | 302 |
| 1744 | 406 | 10 | 211 | 202 | 308 |
| 1745 | 403 | 87 | 206 | 202 | 302 |
| 1746 | 401 | 35 | 209 | 202 | 301 |
| 1747 | 405 | 26 | 209 | 212 | 303 |
| 1748 | 407 | 74 | 210 | 201 | 303 |
| 1749 | 407 | 36 | 212 | 201 | 301 |
| 1750 | 405 | 56 | 209 | 202 | 304 |
| 1751 | 403 | 88 | 209 | 212 | 307 |
| 1752 | 402 | 95 | 204 | 201 | 308 |
| 1753 | 406 | 96 | 209 | 201 | 302 |
| 1754 | 404 | 61 | 202 | 202 | 301 |
| 1755 | 401 | 77 | 204 | 212 | 301 |
| 1756 | 401 | 27 | 212 | 202 | 306 |
| 1757 | 403 | 105 | 212 | 202 | 306 |
| 1758 | 401 | 69 | 210 | 201 | 303 |
| 1759 | 401 | 41 | 202 | 202 | 302 |
| 1760 | 404 | 4 | 201 | 201 | 304 |
| 1761 | 401 | 7 | 204 | 201 | 307 |
| 1762 | 401 | 86 | 204 | 212 | 304 |
| 1763 | 401 | 23 | 205 | 202 | 301 |
| 1764 | 401 | 118 | 203 | 212 | 306 |
| 1765 | 401 | 101 | 211 | 201 | 306 |
| 1766 | 406 | 62 | 205 | 212 | 303 |
| 1767 | 401 | 78 | 204 | 201 | 307 |
| 1768 | 404 | 1 | 201 | 212 | 308 |
| 1769 | 401 | 70 | 205 | 201 | 302 |
| 1770 | 401 | 50 | 212 | 202 | 307 |
| 1771 | 407 | 91 | 206 | 212 | 307 |
| 1772 | 404 | 51 | 211 | 201 | 307 |
| 1773 | 401 | 125 | 201 | 212 | 308 |
| 1774 | 403 | 102 | 201 | 201 | 308 |
| 1775 | 402 | 45 | 205 | 201 | 303 |
| 1776 | 401 | 88 | 210 | 202 | 306 |
| 1777 | 401 | 88 | 210 | 201 | 301 |
| 1778 | 401 | 85 | 211 | 212 | 301 |
| 1779 | 404 | 15 | 211 | 212 | 302 |
| 1780 | 407 | 67 | 211 | 201 | 302 |
| 1781 | 403 | 40 | 210 | 202 | 306 |
| 1782 | 407 | 30 | 210 | 201 | 305 |
| 1783 | 401 | 53 | 204 | 201 | 303 |
| 1784 | 401 | 48 | 212 | 202 | 303 |
| 1785 | 406 | 122 | 201 | 201 | 308 |
| 1786 | 401 | 123 | 201 | 201 | 307 |
| 1787 | 407 | 43 | 212 | 202 | 308 |
| 1788 | 404 | 109 | 212 | 201 | 305 |
| 1789 | 401 | 13 | 204 | 201 | 302 |
| 1790 | 401 | 117 | 211 | 202 | 302 |
| 1791 | 406 | 5 | 205 | 201 | 302 |
| 1792 | 402 | 124 | 203 | 201 | 308 |
| 1793 | 401 | 98 | 208 | 201 | 308 |
| 1794 | 406 | 109 | 204 | 212 | 303 |
| 1795 | 401 | 52 | 201 | 202 | 306 |
| 1796 | 406 | 42 | 205 | 212 | 308 |
| 1797 | 407 | 126 | 212 | 202 | 307 |
| 1798 | 403 | 119 | 212 | 212 | 302 |
| 1799 | 403 | 49 | 209 | 201 | 307 |
| 1800 | 401 | 100 | 212 | 212 | 308 |
| 1801 | 402 | 21 | 212 | 202 | 301 |
| 1802 | 401 | 91 | 201 | 201 | 305 |
| 1803 | 403 | 2 | 201 | 201 | 308 |
| 1804 | 401 | 71 | 211 | 202 | 306 |
| 1805 | 401 | 110 | 207 | 201 | 302 |
| 1806 | 407 | 85 | 204 | 212 | 301 |

Compounds having the formula:

TABLE 11

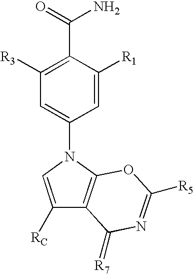

wherein R₁, R₃, R_{C}, R₅, R₆, and R₇ are defined in Table 11:

| Compound | | | | | |
|---|---|---|---|---|---|
| No. | R₁ | R₃ | R_{C} | R₅ | R₇ |
| 1807 | 401 | 17 | 207 | 201 | 308 |
| 1808 | 406 | 33 | 203 | 202 | 302 |
| 1809 | 401 | 35 | 209 | 202 | 301 |
| 1810 | 405 | 23 | 205 | 202 | 301 |
| 1811 | 401 | 76 | 202 | 202 | 306 |
| 1812 | 401 | 126 | 212 | 202 | 307 |
| 1813 | 401 | 96 | 201 | 201 | 308 |
| 1814 | 404 | 28 | 212 | 202 | 302 |
| 1815 | 407 | 96 | 204 | 201 | 308 |
| 1816 | 403 | 43 | 212 | 202 | 308 |
| 1817 | 403 | 11 | 212 | 212 | 306 |
| 1818 | 401 | 98 | 212 | 201 | 307 |
| 1819 | 401 | 86 | 210 | 212 | 303 |
| 1820 | 405 | 40 | 210 | 202 | 306 |
| 1821 | 402 | 44 | 202 | 201 | 307 |
| 1822 | 401 | 109 | 204 | 201 | 303 |
| 1823 | 405 | 121 | 209 | 202 | 308 |
| 1824 | 401 | 104 | 210 | 202 | 307 |
| 1825 | 401 | 47 | 203 | 202 | 306 |
| 1826 | 403 | 109 | 212 | 201 | 305 |
| 1827 | 401 | 2 | 201 | 201 | 308 |
| 1828 | 405 | 85 | 204 | 212 | 301 |
| 1829 | 401 | 115 | 206 | 201 | 308 |
| 1830 | 404 | 91 | 203 | 201 | 306 |
| 1831 | 403 | 84 | 203 | 212 | 303 |
| 1832 | 401 | 24 | 204 | 201 | 301 |
| 1833 | 406 | 78 | 204 | 201 | 307 |
| 1834 | 402 | 88 | 204 | 202 | 306 |
| 1835 | 404 | 96 | 212 | 212 | 307 |
| 1836 | 401 | 15 | 211 | 212 | 302 |
| 1837 | 404 | 120 | 201 | 201 | 304 |
| 1838 | 402 | 34 | 205 | 201 | 304 |
| 1839 | 405 | 41 | 202 | 202 | 302 |
| 1840 | 407 | 88 | 210 | 202 | 306 |
| 1841 | 404 | 70 | 205 | 201 | 302 |
| 1842 | 404 | 85 | 212 | 212 | 307 |
| 1843 | 405 | 102 | 201 | 201 | 308 |
| 1844 | 403 | 4 | 201 | 201 | 304 |
| 1845 | 404 | 114 | 212 | 202 | 304 |
| 1846 | 401 | 54 | 212 | 201 | 302 |
| 1847 | 401 | 68 | 204 | 212 | 304 |
| 1848 | 405 | 105 | 212 | 202 | 306 |
| 1849 | 403 | 77 | 204 | 212 | 301 |
| 1850 | 405 | 16 | 205 | 201 | 306 |
| 1851 | 401 | 80 | 204 | 212 | 306 |
| 1852 | 402 | 109 | 207 | 201 | 304 |
| 1853 | 404 | 109 | 207 | 201 | 306 |
| 1854 | 401 | 74 | 210 | 201 | 303 |
| 1855 | 402 | 86 | 204 | 212 | 304 |
| 1856 | 401 | 86 | 205 | 201 | 303 |
| 1857 | 403 | 57 | 202 | 212 | 308 |
| 1858 | 401 | 39 | 204 | 212 | 302 |
| 1859 | 401 | 93 | 205 | 212 | 307 |
| 1860 | 401 | 81 | 210 | 212 | 301 |
| 1861 | 401 | 112 | 212 | 201 | 304 |
| 1862 | 405 | 129 | 211 | 202 | 302 |
| 1863 | 401 | 50 | 212 | 202 | 307 |
| 1864 | 403 | 117 | 211 | 202 | 302 |
| 1865 | 404 | 52 | 201 | 202 | 306 |
| 1866 | 402 | 67 | 211 | 201 | 302 |
| 1867 | 401 | 88 | 210 | 201 | 301 |
| 1868 | 405 | 96 | 210 | 212 | 305 |
| 1869 | 401 | 109 | 204 | 212 | 303 |
| 1870 | 406 | 92 | 204 | 212 | 301 |
| 1871 | 401 | 85 | 210 | 202 | 303 |
| 1872 | 401 | 88 | 211 | 201 | 306 |
| 1873 | 404 | 42 | 205 | 212 | 308 |
| 1874 | 405 | 89 | 206 | 202 | 306 |
| 1875 | 403 | 36 | 212 | 201 | 301 |
| 1876 | 401 | 6 | 210 | 202 | 306 |
| 1877 | 407 | 59 | 206 | 201 | 306 |
| 1878 | 402 | 61 | 202 | 202 | 301 |
| 1879 | 403 | 96 | 211 | 201 | 301 |
| 1880 | 404 | 91 | 212 | 201 | 304 |
| 1881 | 406 | 123 | 201 | 201 | 307 |
| 1882 | 407 | 5 | 205 | 201 | 302 |
| 1883 | 403 | 71 | 211 | 202 | 306 |
| 1884 | 404 | 88 | 206 | 202 | 303 |
| 1885 | 401 | 128 | 212 | 201 | 303 |
| 1886 | 401 | 48 | 212 | 202 | 303 |
| 1887 | 407 | 96 | 209 | 201 | 302 |
| 1888 | 401 | 12 | 207 | 201 | 303 |
| 1889 | 401 | 85 | 204 | 202 | 301 |
| 1890 | 404 | 73 | 210 | 202 | 308 |
| 1891 | 406 | 101 | 203 | 201 | 301 |
| 1892 | 407 | 103 | 205 | 201 | 301 |
| 1893 | 402 | 88 | 209 | 212 | 307 |
| 1894 | 401 | 66 | 201 | 212 | 304 |
| 1895 | 405 | 20 | 201 | 201 | 305 |
| 1896 | 407 | 45 | 205 | 201 | 303 |
| 1897 | 401 | 113 | 210 | 201 | 307 |
| 1898 | 405 | 51 | 211 | 201 | 307 |
| 1899 | 401 | 46 | 204 | 202 | 308 |
| 1900 | 401 | 10 | 211 | 202 | 308 |
| 1901 | 401 | 94 | 201 | 212 | 304 |
| 1902 | 401 | 18 | 202 | 202 | 306 |
| 1903 | 403 | 22 | 211 | 201 | 308 |
| 1904 | 406 | 7 | 204 | 201 | 307 |
| 1905 | 401 | 96 | 212 | 201 | 301 |
| 1906 | 401 | 124 | 203 | 201 | 308 |
| 1907 | 402 | 32 | 211 | 212 | 301 |
| 1908 | 401 | 3 | 203 | 202 | 302 |
| 1909 | 403 | 55 | 204 | 201 | 302 |
| 1910 | 401 | 86 | 212 | 212 | 301 |
| 1911 | 401 | 88 | 208 | 212 | 301 |
| 1912 | 402 | 101 | 201 | 212 | 308 |
| 1913 | 406 | 95 | 204 | 201 | 308 |
| 1914 | 401 | 109 | 207 | 201 | 302 |
| 1915 | 403 | 30 | 210 | 201 | 305 |
| 1916 | 402 | 8 | 204 | 212 | 301 |
| 1917 | 401 | 98 | 205 | 201 | 302 |
| 1918 | 401 | 56 | 209 | 202 | 304 |
| 1919 | 404 | 13 | 204 | 201 | 302 |
| 1920 | 403 | 98 | 208 | 201 | 308 |
| 1921 | 406 | 79 | 206 | 212 | 301 |
| 1922 | 406 | 96 | 201 | 201 | 304 |
| 1923 | 403 | 75 | 204 | 202 | 302 |
| 1924 | 401 | 108 | 205 | 212 | 302 |
| 1925 | 401 | 98 | 208 | 201 | 301 |
| 1926 | 401 | 19 | 202 | 201 | 308 |
| 1927 | 401 | 91 | 206 | 212 | 307 |
| 1928 | 403 | 98 | 209 | 201 | 307 |
| 1929 | 407 | 29 | 210 | 202 | 306 |
| 1930 | 401 | 85 | 211 | 212 | 301 |
| 1931 | 405 | 38 | 206 | 201 | 305 |
| 1932 | 404 | 107 | 204 | 201 | 302 |
| 1933 | 401 | 119 | 212 | 212 | 302 |
| 1934 | 402 | 110 | 207 | 201 | 302 |
| 1935 | 407 | 116 | 205 | 202 | 301 |
| 1936 | 402 | 87 | 206 | 202 | 302 |
| 1937 | 402 | 101 | 207 | 212 | 301 |
| 1938 | 407 | 72 | 204 | 202 | 305 |
| 1939 | 401 | 60 | 202 | 201 | 301 |
| 1940 | 401 | 65 | 212 | 212 | 306 |
| 1941 | 402 | 101 | 205 | 202 | 302 |
| 1942 | 406 | 53 | 204 | 201 | 303 |
| 1943 | 407 | 122 | 201 | 201 | 308 |
| 1944 | 402 | 9 | 212 | 212 | 305 |
| 1945 | 406 | 101 | 212 | 202 | 307 |
| 1946 | 401 | 106 | 205 | 212 | 307 |
| 1947 | 405 | 109 | 201 | 212 | 301 |
| 1948 | 401 | 21 | 212 | 202 | 301 |
| 1949 | 401 | 86 | 201 | 202 | 301 |
| 1950 | 402 | 99 | 203 | 202 | 307 |
| 1951 | 401 | 127 | 210 | 201 | 301 |
| 1952 | 401 | 100 | 212 | 212 | 308 |
| 1953 | 401 | 26 | 209 | 212 | 303 |
| 1954 | 406 | 63 | 210 | 202 | 308 |
| 1955 | 401 | 91 | 210 | 201 | 308 |
| 1956 | 406 | 101 | 211 | 201 | 306 |
| 1957 | 404 | 130 | 206 | 202 | 306 |
| 1958 | 407 | 118 | 203 | 212 | 306 |
| 1959 | 401 | 85 | 204 | 212 | 308 |
| 1960 | 402 | 27 | 212 | 202 | 306 |
| 1961 | 406 | 91 | 201 | 201 | 305 |
| 1962 | 401 | 49 | 209 | 201 | 307 |
| 1963 | 406 | 64 | 212 | 201 | 308 |
| 1964 | 405 | 31 | 208 | 212 | 305 |
| 1965 | 405 | 82 | 209 | 202 | 302 |
| 1966 | 401 | 14 | 203 | 201 | 308 |
| 1967 | 401 | 98 | 204 | 212 | 301 |
| 1968 | 407 | 91 | 209 | 212 | 302 |
| 1969 | 401 | 86 | 201 | 212 | 302 |
| 1970 | 407 | 83 | 211 | 212 | 303 |
| 1971 | 401 | 37 | 204 | 202 | 306 |
| 1972 | 406 | 1 | 201 | 212 | 308 |
| 1973 | 401 | 69 | 210 | 201 | 303 |
| 1974 | 401 | 90 | 205 | 212 | 305 |
| 1975 | 407 | 111 | 204 | 202 | 308 |
| 1976 | 401 | 88 | 206 | 212 | 301 |
| 1977 | 407 | 25 | 204 | 202 | 306 |
| 1978 | 401 | 97 | 210 | 201 | 304 |
| 1979 | 404 | 125 | 201 | 212 | 308 |
| 1980 | 407 | 62 | 205 | 212 | 303 |
| 1981 | 406 | 58 | 202 | 212 | 306 |

Compounds having the formula:

TABLE 12

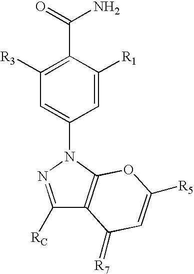

wherein R₁, R₃, R_{C}, R₅, R₆, and R₇ are defined in Table 12:

| Compound | | | | | |
|---|---|---|---|---|---|
| No. | R₁ | R₃ | R_{C} | R₅ | R₇ |
| 1982 | 402 | 32 | 211 | 212 | 301 |
| 1983 | 407 | 86 | 210 | 212 | 303 |
| 1984 | 404 | 86 | 204 | 212 | 304 |
| 1985 | 404 | 96 | 209 | 201 | 302 |
| 1986 | 402 | 27 | 212 | 202 | 306 |
| 1987 | 402 | 109 | 207 | 201 | 306 |
| 1988 | 401 | 67 | 211 | 201 | 302 |
| 1989 | 401 | 39 | 204 | 212 | 302 |
| 1990 | 405 | 96 | 201 | 201 | 304 |
| 1991 | 404 | 37 | 204 | 202 | 306 |
| 1992 | 406 | 54 | 212 | 201 | 302 |
| 1993 | 401 | 55 | 204 | 201 | 302 |
| 1994 | 401 | 85 | 204 | 202 | 301 |
| 1995 | 402 | 106 | 205 | 212 | 307 |
| 1996 | 401 | 96 | 204 | 201 | 308 |
| 1997 | 404 | 88 | 210 | 202 | 306 |
| 1998 | 402 | 70 | 205 | 201 | 302 |
| 1999 | 405 | 109 | 207 | 201 | 304 |
| 2000 | 402 | 6 | 210 | 202 | 306 |
| 2001 | 401 | 58 | 202 | 212 | 306 |
| 2002 | 405 | 105 | 212 | 202 | 306 |
| 2003 | 404 | 25 | 204 | 202 | 306 |
| 2004 | 401 | 101 | 201 | 212 | 308 |
| 2005 | 405 | 74 | 210 | 201 | 303 |
| 2006 | 406 | 116 | 205 | 202 | 301 |
| 2007 | 407 | 119 | 212 | 212 | 302 |
| 2008 | 401 | 104 | 210 | 202 | 307 |
| 2009 | 401 | 21 | 212 | 202 | 301 |
| 2010 | 403 | 93 | 205 | 212 | 307 |
| 2011 | 401 | 48 | 212 | 202 | 303 |
| 2012 | 401 | 96 | 212 | 201 | 301 |
| 2013 | 401 | 69 | 210 | 201 | 303 |
| 2014 | 401 | 117 | 211 | 202 | 302 |
| 2015 | 401 | 110 | 207 | 201 | 302 |
| 2016 | 401 | 78 | 204 | 201 | 307 |
| 2017 | 401 | 34 | 205 | 201 | 304 |
| 2018 | 401 | 53 | 204 | 201 | 303 |
| 2019 | 405 | 19 | 202 | 201 | 308 |
| 2020 | 401 | 2 | 201 | 201 | 308 |
| 2021 | 404 | 96 | 212 | 212 | 307 |
| 2022 | 402 | 107 | 204 | 201 | 302 |
| 2023 | 401 | 15 | 211 | 212 | 302 |
| 2024 | 405 | 85 | 204 | 212 | 301 |
| 2025 | 401 | 65 | 212 | 212 | 306 |
| 2026 | 403 | 101 | 212 | 202 | 307 |
| 2027 | 401 | 98 | 205 | 201 | 302 |
| 2028 | 401 | 88 | 208 | 212 | 301 |
| 2029 | 406 | 66 | 201 | 212 | 304 |
| 2030 | 401 | 97 | 210 | 201 | 304 |
| 2031 | 403 | 101 | 205 | 202 | 302 |
| 2032 | 405 | 79 | 206 | 212 | 301 |
| 2033 | 404 | 36 | 212 | 201 | 301 |
| 2034 | 407 | 42 | 205 | 212 | 308 |
| 2035 | 403 | 61 | 202 | 202 | 301 |
| 2036 | 402 | 109 | 201 | 212 | 301 |
| 2037 | 407 | 43 | 212 | 202 | 308 |
| 2038 | 401 | 118 | 203 | 212 | 306 |
| 2039 | 403 | 101 | 207 | 212 | 301 |
| 2040 | 405 | 85 | 211 | 212 | 301 |
| 2041 | 401 | 17 | 207 | 201 | 308 |
| 2042 | 401 | 35 | 209 | 202 | 301 |
| 2043 | 402 | 11 | 212 | 212 | 306 |
| 2044 | 402 | 108 | 205 | 212 | 302 |
| 2045 | 403 | 88 | 211 | 201 | 306 |
| 2046 | 407 | 98 | 209 | 201 | 307 |
| 2047 | 406 | 122 | 201 | 201 | 308 |
| 2048 | 403 | 125 | 201 | 212 | 308 |
| 2049 | 401 | 1 | 201 | 212 | 308 |
| 2050 | 401 | 51 | 211 | 201 | 307 |
| 2051 | 405 | 91 | 203 | 201 | 306 |
| 2052 | 406 | 90 | 205 | 212 | 305 |
| 2053 | 401 | 7 | 204 | 201 | 307 |
| 2054 | 405 | 86 | 205 | 201 | 303 |
| 2055 | 401 | 96 | 211 | 201 | 301 |
| 2056 | 407 | 41 | 202 | 202 | 302 |
| 2057 | 401 | 28 | 212 | 202 | 302 |
| 2058 | 405 | 86 | 201 | 212 | 302 |
| 2059 | 401 | 10 | 211 | 202 | 308 |
| 2060 | 406 | 16 | 205 | 201 | 306 |
| 2061 | 406 | 77 | 204 | 212 | 301 |
| 2062 | 401 | 52 | 201 | 202 | 306 |
| 2063 | 406 | 109 | 204 | 201 | 303 |
| 2064 | 406 | 94 | 201 | 212 | 304 |
| 2065 | 401 | 113 | 210 | 201 | 307 |
| 2066 | 407 | 75 | 204 | 202 | 302 |
| 2067 | 401 | 112 | 212 | 201 | 304 |
| 2068 | 401 | 84 | 203 | 212 | 303 |
| 2069 | 404 | 85 | 204 | 212 | 308 |
| 2070 | 401 | 56 | 209 | 202 | 304 |
| 2071 | 401 | 109 | 207 | 201 | 302 |
| 2072 | 402 | 124 | 203 | 201 | 308 |
| 2073 | 401 | 126 | 212 | 202 | 307 |
| 2074 | 404 | 8 | 204 | 212 | 301 |
| 2075 | 403 | 101 | 211 | 201 | 306 |
| 2076 | 404 | 88 | 210 | 201 | 301 |
| 2077 | 401 | 85 | 212 | 212 | 307 |
| 2078 | 405 | 64 | 212 | 201 | 308 |
| 2079 | 407 | 98 | 208 | 201 | 308 |
| 2080 | 402 | 38 | 206 | 201 | 305 |
| 2081 | 406 | 109 | 204 | 212 | 303 |
| 2082 | 401 | 80 | 204 | 212 | 306 |
| 2083 | 404 | 18 | 202 | 202 | 306 |
| 2084 | 401 | 60 | 202 | 201 | 301 |
| 2085 | 405 | 24 | 204 | 201 | 301 |
| 2086 | 402 | 76 | 202 | 202 | 306 |
| 2087 | 403 | 46 | 204 | 202 | 308 |
| 2088 | 401 | 33 | 203 | 202 | 302 |
| 2089 | 407 | 31 | 208 | 212 | 305 |
| 2090 | 402 | 87 | 206 | 202 | 302 |
| 2091 | 405 | 71 | 211 | 202 | 306 |
| 2092 | 405 | 115 | 206 | 201 | 308 |
| 2093 | 406 | 96 | 201 | 201 | 308 |
| 2094 | 403 | 98 | 204 | 212 | 301 |
| 2095 | 401 | 123 | 201 | 201 | 307 |
| 2096 | 405 | 101 | 203 | 201 | 301 |
| 2097 | 407 | 9 | 212 | 212 | 305 |
| 2098 | 407 | 91 | 201 | 201 | 305 |
| 2099 | 405 | 111 | 204 | 202 | 308 |
| 2100 | 403 | 72 | 204 | 202 | 305 |
| 2101 | 402 | 44 | 202 | 201 | 307 |
| 2102 | 406 | 128 | 212 | 201 | 303 |
| 2103 | 404 | 127 | 210 | 201 | 301 |
| 2104 | 401 | 91 | 206 | 212 | 307 |
| 2105 | 401 | 49 | 209 | 201 | 307 |
| 2106 | 403 | 30 | 210 | 201 | 305 |
| 2107 | 401 | 82 | 209 | 202 | 302 |
| 2108 | 401 | 130 | 206 | 202 | 306 |
| 2109 | 404 | 91 | 209 | 212 | 302 |
| 2110 | 401 | 63 | 210 | 202 | 308 |
| 2111 | 401 | 40 | 210 | 202 | 306 |
| 2112 | 401 | 29 | 210 | 202 | 306 |
| 2113 | 401 | 85 | 210 | 202 | 303 |
| 2114 | 401 | 73 | 210 | 202 | 308 |
| 2115 | 406 | 4 | 201 | 201 | 304 |
| 2116 | 401 | 86 | 212 | 212 | 301 |
| 2117 | 404 | 81 | 210 | 212 | 301 |
| 2118 | 401 | 98 | 212 | 201 | 307 |
| 2119 | 401 | 88 | 209 | 212 | 307 |
| 2120 | 407 | 57 | 202 | 212 | 308 |
| 2121 | 404 | 86 | 201 | 202 | 301 |
| 2122 | 401 | 20 | 201 | 201 | 305 |
| 2123 | 403 | 14 | 203 | 201 | 308 |
| 2124 | 402 | 68 | 204 | 212 | 304 |
| 2125 | 401 | 88 | 206 | 202 | 303 |
| 2126 | 401 | 99 | 203 | 202 | 307 |
| 2127 | 401 | 114 | 212 | 202 | 304 |
| 2128 | 404 | 47 | 203 | 202 | 306 |
| 2129 | 407 | 12 | 207 | 201 | 303 |
| 2130 | 403 | 62 | 205 | 212 | 303 |
| 2131 | 407 | 109 | 212 | 201 | 305 |
| 2132 | 401 | 89 | 206 | 202 | 306 |
| 2133 | 401 | 92 | 204 | 212 | 301 |
| 2134 | 406 | 45 | 205 | 201 | 303 |
| 2135 | 401 | 22 | 211 | 201 | 308 |
| 2136 | 407 | 59 | 206 | 201 | 306 |
| 2137 | 404 | 96 | 210 | 212 | 305 |
| 2138 | 401 | 120 | 201 | 201 | 304 |
| 2139 | 401 | 50 | 212 | 202 | 307 |
| 2140 | 406 | 103 | 205 | 201 | 301 |
| 2141 | 402 | 83 | 211 | 212 | 303 |
| 2142 | 401 | 13 | 204 | 201 | 302 |
| 2143 | 406 | 91 | 210 | 201 | 308 |
| 2144 | 401 | 91 | 212 | 201 | 304 |
| 2145 | 406 | 129 | 211 | 202 | 302 |
| 2146 | 407 | 5 | 205 | 201 | 302 |
| 2147 | 403 | 3 | 203 | 202 | 302 |
| 2148 | 403 | 102 | 201 | 201 | 308 |
| 2149 | 401 | 26 | 209 | 212 | 303 |
| 2150 | 402 | 88 | 206 | 212 | 301 |
| 2151 | 401 | 23 | 205 | 202 | 301 |
| 2152 | 403 | 88 | 204 | 202 | 306 |
| 2153 | 401 | 121 | 209 | 202 | 308 |
| 2154 | 401 | 98 | 208 | 201 | 301 |
| 2155 | 407 | 100 | 212 | 212 | 308 |

Compounds having the formula:

TABLE 13

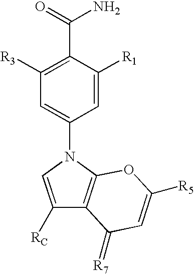

wherein R₁, R₃, R_{C}, R₅, R₆, and R₇ are defined in Table 13:

| Compound No. | R₁ | R₃ | R_{C} | R₅ | R₇ |
|---|---|---|---|---|---|
| 2156 | 402 | 88 | 209 | 212 | 307 |
| 2157 | 407 | 55 | 204 | 201 | 302 |
| 2158 | 404 | 88 | 210 | 201 | 301 |
| 2159 | 401 | 54 | 212 | 201 | 302 |
| 2160 | 404 | 17 | 207 | 201 | 308 |
| 2161 | 402 | 109 | 204 | 201 | 303 |
| 2162 | 402 | 61 | 202 | 202 | 301 |
| 2163 | 401 | 88 | 208 | 212 | 301 |
| 2164 | 401 | 130 | 206 | 202 | 306 |
| 2165 | 405 | 128 | 212 | 201 | 303 |
| 2166 | 404 | 101 | 203 | 201 | 301 |
| 2167 | 406 | 105 | 212 | 202 | 306 |
| 2168 | 401 | 52 | 201 | 202 | 306 |
| 2169 | 401 | 46 | 204 | 202 | 308 |
| 2170 | 402 | 15 | 211 | 212 | 302 |
| 2171 | 401 | 109 | 207 | 201 | 302 |
| 2172 | 404 | 63 | 210 | 202 | 308 |
| 2173 | 402 | 82 | 209 | 202 | 302 |
| 2174 | 405 | 85 | 210 | 202 | 303 |
| 2175 | 402 | 80 | 204 | 212 | 306 |
| 2176 | 401 | 124 | 203 | 201 | 308 |
| 2177 | 405 | 29 | 210 | 202 | 306 |
| 2178 | 404 | 7 | 204 | 201 | 307 |
| 2179 | 401 | 88 | 211 | 201 | 306 |
| 2180 | 405 | 27 | 212 | 202 | 306 |
| 2181 | 406 | 73 | 210 | 202 | 308 |
| 2182 | 407 | 86 | 205 | 201 | 303 |
| 2183 | 401 | 106 | 205 | 212 | 307 |
| 2184 | 401 | 83 | 211 | 212 | 303 |
| 2185 | 403 | 86 | 212 | 212 | 301 |
| 2186 | 401 | 68 | 204 | 212 | 304 |
| 2187 | 401 | 109 | 201 | 212 | 301 |
| 2188 | 401 | 12 | 207 | 201 | 303 |
| 2189 | 401 | 79 | 206 | 212 | 301 |
| 2190 | 401 | 30 | 210 | 201 | 305 |
| 2191 | 401 | 96 | 212 | 201 | 301 |
| 2192 | 401 | 71 | 211 | 202 | 306 |
| 2193 | 401 | 70 | 205 | 201 | 302 |
| 2194 | 405 | 88 | 206 | 202 | 303 |
| 2195 | 401 | 44 | 202 | 201 | 307 |
| 2196 | 404 | 11 | 212 | 212 | 306 |
| 2197 | 402 | 121 | 209 | 202 | 308 |
| 2198 | 401 | 96 | 209 | 201 | 302 |
| 2199 | 405 | 53 | 204 | 201 | 303 |
| 2200 | 401 | 4 | 201 | 201 | 304 |
| 2201 | 403 | 48 | 212 | 202 | 303 |
| 2202 | 401 | 94 | 201 | 212 | 304 |
| 2203 | 401 | 98 | 208 | 201 | 301 |
| 2204 | 406 | 9 | 212 | 212 | 305 |
| 2205 | 401 | 72 | 204 | 202 | 305 |
| 2206 | 403 | 109 | 204 | 212 | 303 |
| 2207 | 405 | 25 | 204 | 202 | 306 |
| 2208 | 404 | 85 | 211 | 212 | 301 |
| 2209 | 407 | 60 | 202 | 201 | 301 |
| 2210 | 403 | 76 | 202 | 202 | 306 |
| 2211 | 402 | 34 | 205 | 201 | 304 |
| 2212 | 407 | 91 | 212 | 201 | 304 |
| 2213 | 401 | 22 | 211 | 201 | 308 |
| 2214 | 403 | 24 | 204 | 201 | 301 |
| 2215 | 405 | 50 | 212 | 202 | 307 |
| 2216 | 401 | 91 | 210 | 201 | 308 |
| 2217 | 401 | 96 | 210 | 212 | 305 |
| 2218 | 402 | 88 | 210 | 202 | 306 |
| 2219 | 402 | 96 | 201 | 201 | 304 |
| 2220 | 403 | 56 | 209 | 202 | 304 |
| 2221 | 407 | 91 | 209 | 212 | 302 |
| 2222 | 406 | 74 | 210 | 201 | 303 |
| 2223 | 403 | 89 | 206 | 202 | 306 |
| 2224 | 401 | 85 | 204 | 202 | 301 |
| 2225 | 401 | 97 | 210 | 201 | 304 |
| 2226 | 405 | 117 | 211 | 202 | 302 |
| 2227 | 406 | 57 | 202 | 212 | 308 |
| 2228 | 401 | 45 | 205 | 201 | 303 |
| 2229 | 405 | 90 | 205 | 212 | 305 |
| 2230 | 401 | 100 | 212 | 212 | 308 |
| 2231 | 407 | 108 | 205 | 212 | 302 |
| 2232 | 401 | 125 | 201 | 212 | 308 |
| 2233 | 405 | 67 | 211 | 201 | 302 |
| 2234 | 401 | 14 | 203 | 201 | 308 |
| 2235 | 406 | 5 | 205 | 201 | 302 |
| 2236 | 406 | 1 | 201 | 212 | 308 |
| 2237 | 401 | 66 | 201 | 212 | 304 |
| 2238 | 406 | 98 | 209 | 201 | 307 |
| 2239 | 406 | 69 | 210 | 201 | 303 |
| 2240 | 401 | 113 | 210 | 201 | 307 |
| 2241 | 407 | 65 | 212 | 212 | 306 |
| 2242 | 401 | 85 | 204 | 212 | 308 |
| 2243 | 401 | 33 | 203 | 202 | 302 |
| 2244 | 404 | 20 | 201 | 201 | 305 |
| 2245 | 401 | 116 | 205 | 202 | 301 |
| 2246 | 401 | 41 | 202 | 202 | 302 |
| 2247 | 402 | 98 | 205 | 201 | 302 |
| 2248 | 401 | 101 | 211 | 201 | 306 |
| 2249 | 404 | 13 | 204 | 201 | 302 |
| 2250 | 403 | 78 | 204 | 201 | 307 |
| 2251 | 404 | 16 | 205 | 201 | 306 |
| 2252 | 401 | 8 | 204 | 212 | 301 |
| 2253 | 405 | 98 | 208 | 201 | 308 |
| 2254 | 407 | 93 | 205 | 212 | 307 |
| 2255 | 402 | 96 | 211 | 201 | 301 |
| 2256 | 406 | 107 | 204 | 201 | 302 |
| 2257 | 401 | 19 | 202 | 201 | 308 |
| 2258 | 404 | 35 | 209 | 202 | 301 |
| 2259 | 401 | 62 | 205 | 212 | 303 |
| 2260 | 405 | 120 | 201 | 201 | 304 |
| 2261 | 402 | 96 | 212 | 212 | 307 |
| 2262 | 403 | 47 | 203 | 202 | 306 |
| 2263 | 401 | 122 | 201 | 201 | 308 |
| 2264 | 407 | 64 | 212 | 201 | 308 |
| 2265 | 402 | 87 | 206 | 202 | 302 |
| 2266 | 405 | 104 | 210 | 202 | 307 |
| 2267 | 405 | 51 | 211 | 201 | 307 |
| 2268 | 406 | 127 | 210 | 201 | 301 |
| 2269 | 403 | 111 | 204 | 202 | 308 |
| 2270 | 401 | 85 | 204 | 212 | 301 |
| 2271 | 405 | 37 | 204 | 202 | 306 |
| 2272 | 407 | 91 | 206 | 212 | 307 |
| 2273 | 407 | 109 | 207 | 201 | 304 |
| 2274 | 405 | 85 | 212 | 212 | 307 |
| 2275 | 403 | 32 | 211 | 212 | 301 |
| 2276 | 402 | 21 | 212 | 202 | 301 |
| 2277 | 406 | 86 | 210 | 212 | 303 |
| 2278 | 404 | 10 | 211 | 202 | 308 |
| 2279 | 401 | 88 | 206 | 212 | 301 |
| 2280 | 401 | 95 | 204 | 201 | 308 |
| 2281 | 403 | 115 | 206 | 201 | 308 |
| 2282 | 401 | 75 | 204 | 202 | 302 |
| 2283 | 401 | 92 | 204 | 212 | 301 |
| 2284 | 404 | 31 | 208 | 212 | 305 |
| 2285 | 401 | 49 | 209 | 201 | 307 |
| 2286 | 401 | 109 | 207 | 201 | 306 |
| 2287 | 401 | 109 | 212 | 201 | 305 |
| 2288 | 401 | 101 | 205 | 202 | 302 |
| 2289 | 401 | 110 | 207 | 201 | 302 |
| 2290 | 406 | 101 | 207 | 212 | 301 |
| 2291 | 401 | 42 | 205 | 212 | 308 |
| 2292 | 404 | 101 | 201 | 212 | 308 |
| 2293 | 401 | 6 | 210 | 202 | 306 |
| 2294 | 401 | 98 | 212 | 201 | 307 |
| 2295 | 407 | 40 | 210 | 202 | 306 |
| 2296 | 404 | 88 | 204 | 202 | 306 |
| 2297 | 401 | 114 | 212 | 202 | 304 |
| 2298 | 403 | 91 | 201 | 201 | 305 |
| 2299 | 402 | 98 | 204 | 212 | 301 |
| 2300 | 401 | 3 | 203 | 202 | 302 |
| 2301 | 401 | 36 | 212 | 201 | 301 |
| 2302 | 401 | 58 | 202 | 212 | 306 |
| 2303 | 404 | 38 | 206 | 201 | 305 |
| 2304 | 407 | 102 | 201 | 201 | 308 |
| 2305 | 403 | 86 | 201 | 202 | 301 |
| 2306 | 407 | 101 | 212 | 202 | 307 |
| 2307 | 401 | 28 | 212 | 202 | 302 |
| 2308 | 401 | 96 | 204 | 201 | 308 |
| 2309 | 406 | 43 | 212 | 202 | 308 |
| 2310 | 401 | 103 | 205 | 201 | 301 |
| 2311 | 407 | 23 | 205 | 202 | 301 |
| 2312 | 404 | 112 | 212 | 201 | 304 |
| 2313 | 401 | 18 | 202 | 202 | 306 |
| 2314 | 401 | 81 | 210 | 212 | 301 |
| 2315 | 406 | 59 | 206 | 201 | 306 |
| 2316 | 402 | 119 | 212 | 212 | 302 |
| 2317 | 401 | 96 | 201 | 201 | 308 |
| 2318 | 406 | 2 | 201 | 201 | 308 |
| 2319 | 401 | 126 | 212 | 202 | 307 |
| 2320 | 406 | 86 | 201 | 212 | 302 |
| 2321 | 407 | 39 | 204 | 212 | 302 |
| 2322 | 403 | 77 | 204 | 212 | 301 |
| 2323 | 403 | 129 | 211 | 202 | 302 |
| 2324 | 401 | 26 | 209 | 212 | 303 |
| 2325 | 402 | 86 | 204 | 212 | 304 |
| 2326 | 401 | 84 | 203 | 212 | 303 |
| 2327 | 403 | 91 | 203 | 201 | 306 |
| 2328 | 401 | 118 | 203 | 212 | 306 |
| 2329 | 401 | 99 | 203 | 202 | 307 |
| 2330 | 407 | 123 | 201 | 201 | 307 |

Compounds having the formula:

TABLE 14

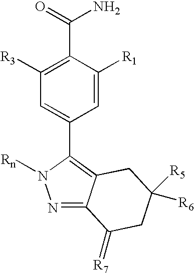

wherein R₁, R₃, Rₙ, R₅, R₆, and R₇ are defined in Table 14:

| Compound No. | R₁ | R₃ | Rₙ | R₅ | R₆ | R₇ |
|---|---|---|---|---|---|---|
| 2331 | 401 | 47 | 203 | 202 | 212 | 306 |
| 2332 | 406 | 96 | 210 | 212 | 212 | 305 |
| 2333 | 401 | 86 | 205 | 201 | 201 | 303 |
| 2334 | 405 | 109 | 212 | 201 | 201 | 305 |
| 2335 | 401 | 86 | 212 | 212 | 212 | 301 |
| 2336 | 401 | 62 | 205 | 212 | 212 | 303 |
| 2337 | 401 | 103 | 205 | 201 | 201 | 301 |
| 2338 | 404 | 88 | 201 | 212 | 212 | 301 |
| 2339 | 407 | 110 | 207 | 201 | 201 | 302 |
| 2340 | 403 | 49 | 202 | 201 | 201 | 307 |
| 2341 | 403 | 29 | 210 | 202 | 212 | 306 |
| 2342 | 401 | 70 | 205 | 201 | 201 | 302 |
| 2343 | 401 | 33 | 203 | 202 | 212 | 302 |
| 2344 | 405 | 46 | 204 | 202 | 212 | 308 |
| 2345 | 402 | 85 | 204 | 212 | 212 | 308 |
| 2346 | 401 | 96 | 212 | 212 | 212 | 307 |
| 2347 | 405 | 111 | 204 | 202 | 212 | 308 |
| 2348 | 401 | 85 | 212 | 212 | 212 | 307 |
| 2349 | 401 | 124 | 203 | 201 | 201 | 308 |
| 2350 | 403 | 6 | 210 | 202 | 212 | 306 |
| 2351 | 401 | 69 | 210 | 201 | 201 | 303 |
| 2352 | 405 | 78 | 204 | 201 | 201 | 307 |
| 2353 | 401 | 108 | 205 | 212 | 212 | 302 |
| 2354 | 404 | 55 | 204 | 201 | 201 | 302 |
| 2355 | 403 | 100 | 212 | 212 | 212 | 308 |
| 2356 | 401 | 45 | 205 | 201 | 201 | 303 |
| 2357 | 406 | 17 | 207 | 201 | 201 | 308 |
| 2358 | 402 | 48 | 212 | 202 | 212 | 303 |
| 2359 | 404 | 86 | 201 | 202 | 212 | 301 |
| 2360 | 401 | 94 | 201 | 212 | 212 | 304 |
| 2361 | 404 | 101 | 205 | 202 | 212 | 302 |
| 2362 | 402 | 121 | 202 | 202 | 212 | 308 |
| 2363 | 405 | 30 | 210 | 201 | 201 | 305 |
| 2364 | 407 | 81 | 210 | 212 | 212 | 301 |
| 2365 | 404 | 88 | 210 | 201 | 201 | 301 |
| 2366 | 404 | 101 | 207 | 212 | 212 | 301 |
| 2367 | 405 | 86 | 210 | 212 | 212 | 303 |
| 2368 | 403 | 82 | 202 | 202 | 212 | 302 |
| 2369 | 404 | 120 | 201 | 201 | 201 | 304 |
| 2370 | 401 | 77 | 204 | 212 | 212 | 301 |
| 2371 | 401 | 14 | 203 | 201 | 201 | 308 |
| 2372 | 405 | 99 | 203 | 202 | 212 | 307 |
| 2373 | 403 | 56 | 202 | 202 | 212 | 304 |
| 2374 | 405 | 98 | 208 | 201 | 201 | 308 |
| 2375 | 401 | 74 | 210 | 201 | 201 | 303 |
| 2376 | 402 | 64 | 212 | 201 | 201 | 308 |
| 2377 | 404 | 8 | 204 | 212 | 212 | 301 |
| 2378 | 401 | 84 | 203 | 212 | 212 | 303 |
| 2379 | 402 | 37 | 204 | 202 | 212 | 306 |
| 2380 | 401 | 88 | 211 | 201 | 201 | 306 |
| 2381 | 403 | 21 | 212 | 202 | 212 | 301 |
| 2382 | 401 | 95 | 204 | 201 | 201 | 308 |
| 2383 | 401 | 7 | 204 | 201 | 201 | 307 |
| 2384 | 401 | 41 | 202 | 202 | 212 | 302 |
| 2385 | 401 | 91 | 202 | 212 | 212 | 302 |
| 2386 | 405 | 91 | 201 | 201 | 201 | 305 |
| 2387 | 401 | 88 | 201 | 202 | 212 | 303 |
| 2388 | 403 | 96 | 201 | 201 | 201 | 308 |
| 2389 | 404 | 91 | 212 | 201 | 201 | 304 |
| 2390 | 402 | 20 | 201 | 201 | 201 | 305 |
| 2391 | 401 | 12 | 207 | 201 | 201 | 303 |
| 2392 | 405 | 51 | 211 | 201 | 201 | 307 |
| 2393 | 401 | 23 | 205 | 202 | 212 | 301 |
| 2394 | 406 | 88 | 204 | 202 | 212 | 306 |
| 2395 | 401 | 32 | 211 | 212 | 212 | 301 |
| 2396 | 401 | 96 | 212 | 201 | 201 | 301 |
| 2397 | 404 | 91 | 210 | 201 | 201 | 308 |
| 2398 | 405 | 25 | 204 | 202 | 212 | 306 |
| 2399 | 403 | 59 | 201 | 201 | 201 | 306 |
| 2400 | 401 | 98 | 202 | 201 | 201 | 307 |
| 2401 | 407 | 76 | 202 | 202 | 212 | 306 |
| 2402 | 402 | 22 | 211 | 201 | 201 | 308 |
| 2403 | 403 | 116 | 205 | 202 | 212 | 301 |
| 2404 | 404 | 75 | 204 | 202 | 212 | 302 |
| 2405 | 406 | 31 | 208 | 212 | 212 | 305 |
| 2406 | 407 | 34 | 205 | 201 | 201 | 304 |
| 2407 | 403 | 88 | 210 | 202 | 212 | 306 |
| 2408 | 404 | 90 | 205 | 212 | 212 | 305 |
| 2409 | 401 | 85 | 210 | 202 | 212 | 303 |
| 2410 | 401 | 15 | 211 | 212 | 212 | 302 |
| 2411 | 407 | 86 | 201 | 212 | 212 | 302 |
| 2412 | 401 | 91 | 203 | 201 | 201 | 306 |
| 2413 | 401 | 36 | 212 | 201 | 201 | 301 |
| 2414 | 404 | 127 | 210 | 201 | 201 | 301 |
| 2415 | 406 | 109 | 207 | 201 | 201 | 304 |
| 2416 | 407 | 115 | 201 | 201 | 201 | 308 |
| 2417 | 402 | 88 | 202 | 212 | 212 | 307 |
| 2418 | 401 | 3 | 203 | 202 | 212 | 302 |
| 2419 | 405 | 98 | 212 | 201 | 201 | 307 |
| 2420 | 407 | 91 | 201 | 212 | 212 | 307 |
| 2421 | 401 | 105 | 212 | 202 | 212 | 306 |
| 2422 | 405 | 112 | 212 | 201 | 201 | 304 |
| 2423 | 401 | 109 | 201 | 212 | 212 | 301 |
| 2424 | 401 | 98 | 208 | 201 | 201 | 301 |
| 2425 | 401 | 60 | 202 | 201 | 201 | 301 |
| 2426 | 401 | 130 | 201 | 202 | 212 | 306 |
| 2427 | 403 | 5 | 205 | 201 | 201 | 302 |
| 2428 | 406 | 4 | 201 | 201 | 201 | 304 |
| 2429 | 401 | 114 | 212 | 202 | 212 | 304 |
| 2430 | 401 | 101 | 201 | 212 | 212 | 308 |
| 2431 | 402 | 52 | 201 | 202 | 212 | 306 |
| 2432 | 401 | 101 | 212 | 202 | 212 | 307 |
| 2433 | 403 | 102 | 201 | 201 | 201 | 308 |
| 2434 | 401 | 129 | 211 | 202 | 212 | 302 |
| 2435 | 401 | 9 | 212 | 212 | 212 | 305 |
| 2436 | 402 | 123 | 201 | 201 | 201 | 307 |
| 2437 | 406 | 97 | 210 | 201 | 201 | 304 |
| 2438 | 401 | 53 | 204 | 201 | 201 | 303 |
| 2439 | 403 | 79 | 201 | 212 | 212 | 301 |
| 2440 | 402 | 2 | 201 | 201 | 201 | 308 |
| 2441 | 401 | 73 | 210 | 202 | 212 | 308 |
| 2442 | 401 | 106 | 205 | 212 | 212 | 307 |
| 2443 | 404 | 96 | 201 | 201 | 201 | 304 |
| 2444 | 403 | 39 | 204 | 212 | 212 | 302 |
| 2445 | 406 | 61 | 202 | 202 | 212 | 301 |
| 2446 | 406 | 57 | 202 | 212 | 212 | 308 |
| 2447 | 403 | 93 | 205 | 212 | 212 | 307 |
| 2448 | 401 | 122 | 201 | 201 | 201 | 308 |
| 2449 | 401 | 18 | 202 | 202 | 212 | 306 |
| 2450 | 401 | 66 | 201 | 212 | 212 | 304 |
| 2451 | 401 | 92 | 204 | 212 | 212 | 301 |
| 2452 | 403 | 44 | 202 | 201 | 201 | 307 |
| 2453 | 407 | 35 | 202 | 202 | 212 | 301 |
| 2454 | 401 | 101 | 211 | 201 | 201 | 306 |
| 2455 | 405 | 119 | 212 | 212 | 212 | 302 |
| 2456 | 404 | 117 | 211 | 202 | 212 | 302 |
| 2457 | 401 | 26 | 202 | 212 | 212 | 303 |
| 2458 | 402 | 98 | 205 | 201 | 201 | 302 |
| 2459 | 407 | 63 | 210 | 202 | 212 | 308 |
| 2460 | 402 | 65 | 212 | 212 | 212 | 306 |
| 2461 | 402 | 109 | 207 | 201 | 201 | 302 |
| 2462 | 407 | 104 | 210 | 202 | 212 | 307 |
| 2463 | 401 | 109 | 207 | 201 | 201 | 306 |
| 2464 | 401 | 28 | 212 | 202 | 212 | 302 |
| 2465 | 402 | 11 | 212 | 212 | 212 | 306 |
| 2466 | 406 | 88 | 208 | 212 | 212 | 301 |
| 2467 | 407 | 54 | 212 | 201 | 201 | 302 |
| 2468 | 402 | 58 | 202 | 212 | 212 | 306 |
| 2469 | 406 | 83 | 211 | 212 | 212 | 303 |
| 2470 | 401 | 89 | 201 | 202 | 212 | 306 |
| 2471 | 405 | 107 | 204 | 201 | 201 | 302 |
| 2472 | 401 | 109 | 204 | 212 | 212 | 303 |
| 2473 | 401 | 96 | 211 | 201 | 201 | 301 |
| 2474 | 402 | 50 | 212 | 202 | 212 | 307 |
| 2475 | 401 | 118 | 203 | 212 | 212 | 306 |
| 2476 | 401 | 101 | 203 | 201 | 201 | 301 |
| 2477 | 401 | 80 | 204 | 212 | 212 | 306 |
| 2478 | 406 | 96 | 204 | 201 | 201 | 308 |
| 2479 | 401 | 40 | 210 | 202 | 212 | 306 |
| 2480 | 406 | 96 | 202 | 201 | 201 | 302 |
| 2481 | 404 | 98 | 204 | 212 | 212 | 301 |
| 2482 | 407 | 125 | 201 | 212 | 212 | 308 |
| 2483 | 401 | 68 | 204 | 212 | 212 | 304 |
| 2484 | 402 | 19 | 202 | 201 | 201 | 308 |
| 2485 | 406 | 85 | 204 | 202 | 212 | 301 |
| 2486 | 401 | 16 | 205 | 201 | 201 | 306 |
| 2487 | 406 | 86 | 204 | 212 | 212 | 304 |
| 2488 | 405 | 128 | 212 | 201 | 201 | 303 |
| 2489 | 405 | 38 | 201 | 201 | 201 | 305 |
| 2490 | 401 | 72 | 204 | 202 | 212 | 305 |
| 2491 | 401 | 10 | 211 | 202 | 212 | 308 |
| 2492 | 407 | 71 | 211 | 202 | 212 | 306 |
| 2493 | 401 | 85 | 211 | 212 | 212 | 301 |
| 2494 | 407 | 126 | 212 | 202 | 212 | 307 |
| 2495 | 401 | 87 | 201 | 202 | 212 | 302 |
| 2496 | 406 | 13 | 204 | 201 | 201 | 302 |
| 2497 | 401 | 24 | 204 | 201 | 201 | 301 |
| 2498 | 401 | 85 | 204 | 212 | 212 | 301 |
| 2499 | 407 | 1 | 201 | 212 | 212 | 308 |
| 2500 | 401 | 27 | 212 | 202 | 212 | 306 |
| 2501 | 407 | 67 | 211 | 201 | 201 | 302 |
| 2502 | 401 | 43 | 212 | 202 | 212 | 308 |
| 2503 | 404 | 109 | 204 | 201 | 201 | 303 |
| 2504 | 407 | 113 | 210 | 201 | 201 | 307 |
| 2505 | 406 | 42 | 205 | 212 | 212 | 308 |

Compounds having the formula:

TABLE 15

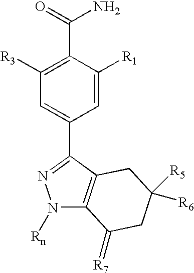

wherein R₁, R₃, Rₙ, R₅, R₆, and R₇ are defined in Table 15:

| Compound No. | R₁ | R₃ | Rₙ | R₅ | R₆ | R₇ |
|---|---|---|---|---|---|---|
| 2506 | 406 | 103 | 205 | 201 | 201 | 301 |
| 2507 | 401 | 55 | 204 | 201 | 201 | 302 |
| 2508 | 405 | 101 | 203 | 201 | 201 | 301 |
| 2509 | 401 | 50 | 212 | 202 | 212 | 307 |
| 2510 | 401 | 98 | 212 | 201 | 201 | 307 |
| 2511 | 401 | 91 | 201 | 212 | 212 | 307 |
| 2512 | 404 | 88 | 210 | 202 | 212 | 306 |
| 2513 | 407 | 43 | 212 | 202 | 212 | 308 |
| 2514 | 403 | 101 | 212 | 202 | 212 | 307 |
| 2515 | 403 | 88 | 204 | 202 | 212 | 306 |
| 2516 | 401 | 118 | 203 | 212 | 212 | 306 |
| 2517 | 401 | 28 | 212 | 202 | 212 | 302 |
| 2518 | 405 | 74 | 210 | 201 | 201 | 303 |
| 2519 | 402 | 44 | 202 | 201 | 201 | 307 |
| 2520 | 401 | 73 | 210 | 202 | 212 | 308 |
| 2521 | 405 | 79 | 201 | 212 | 212 | 301 |
| 2522 | 401 | 65 | 212 | 212 | 212 | 306 |
| 2523 | 401 | 91 | 212 | 201 | 201 | 304 |
| 2524 | 403 | 61 | 202 | 202 | 212 | 301 |
| 2525 | 401 | 23 | 205 | 202 | 212 | 301 |
| 2526 | 405 | 105 | 212 | 202 | 212 | 306 |
| 2527 | 401 | 34 | 205 | 201 | 201 | 304 |
| 2528 | 404 | 18 | 202 | 202 | 212 | 306 |
| 2529 | 403 | 101 | 211 | 201 | 201 | 306 |
| 2530 | 401 | 88 | 201 | 202 | 212 | 303 |
| 2531 | 406 | 4 | 201 | 201 | 201 | 304 |
| 2532 | 402 | 106 | 205 | 212 | 212 | 307 |
| 2533 | 404 | 96 | 212 | 212 | 212 | 307 |
| 2534 | 401 | 121 | 202 | 202 | 212 | 308 |
| 2535 | 404 | 25 | 204 | 202 | 212 | 306 |
| 2536 | 402 | 108 | 205 | 212 | 212 | 302 |
| 2537 | 405 | 91 | 203 | 201 | 201 | 306 |
| 2538 | 407 | 91 | 201 | 201 | 201 | 305 |
| 2539 | 404 | 86 | 204 | 212 | 212 | 304 |
| 2540 | 404 | 88 | 210 | 201 | 201 | 301 |
| 2541 | 405 | 115 | 201 | 201 | 201 | 308 |
| 2542 | 403 | 88 | 211 | 201 | 201 | 306 |
| 2543 | 404 | 91 | 202 | 212 | 212 | 302 |
| 2544 | 401 | 82 | 202 | 202 | 212 | 302 |
| 2545 | 401 | 98 | 208 | 201 | 201 | 301 |
| 2546 | 405 | 85 | 211 | 212 | 212 | 301 |
| 2547 | 403 | 30 | 210 | 201 | 201 | 305 |
| 2548 | 405 | 64 | 212 | 201 | 201 | 308 |
| 2549 | 401 | 48 | 212 | 202 | 212 | 303 |
| 2550 | 402 | 27 | 212 | 202 | 212 | 306 |
| 2551 | 404 | 47 | 203 | 202 | 212 | 306 |
| 2552 | 401 | 29 | 210 | 202 | 212 | 306 |
| 2553 | 402 | 124 | 203 | 201 | 201 | 308 |
| 2554 | 401 | 1 | 201 | 212 | 212 | 308 |
| 2555 | 403 | 3 | 203 | 202 | 212 | 302 |
| 2556 | 401 | 51 | 211 | 201 | 201 | 307 |
| 2557 | 401 | 101 | 201 | 212 | 212 | 308 |
| 2558 | 401 | 52 | 201 | 202 | 212 | 306 |
| 2559 | 401 | 88 | 208 | 212 | 212 | 301 |
| 2560 | 405 | 86 | 205 | 201 | 201 | 303 |
| 2561 | 401 | 67 | 211 | 201 | 201 | 302 |
| 2562 | 403 | 62 | 205 | 212 | 212 | 303 |
| 2563 | 404 | 81 | 210 | 212 | 212 | 301 |
| 2564 | 402 | 88 | 201 | 212 | 212 | 301 |
| 2565 | 401 | 112 | 212 | 201 | 201 | 304 |
| 2566 | 405 | 85 | 204 | 212 | 212 | 301 |
| 2567 | 401 | 17 | 207 | 201 | 201 | 308 |
| 2568 | 406 | 94 | 201 | 212 | 212 | 304 |
| 2569 | 401 | 80 | 204 | 212 | 212 | 306 |
| 2570 | 401 | 110 | 207 | 201 | 201 | 302 |
| 2571 | 404 | 96 | 210 | 212 | 212 | 305 |
| 2572 | 405 | 71 | 211 | 202 | 212 | 306 |
| 2573 | 403 | 14 | 203 | 201 | 201 | 308 |
| 2574 | 401 | 22 | 211 | 201 | 201 | 308 |
| 2575 | 407 | 5 | 205 | 201 | 201 | 302 |
| 2576 | 402 | 109 | 201 | 212 | 212 | 301 |
| 2577 | 403 | 46 | 204 | 202 | 212 | 308 |
| 2578 | 404 | 37 | 204 | 202 | 212 | 306 |
| 2579 | 406 | 109 | 204 | 212 | 212 | 303 |
| 2580 | 407 | 109 | 212 | 201 | 201 | 305 |
| 2581 | 403 | 101 | 205 | 202 | 212 | 302 |
| 2582 | 404 | 86 | 201 | 202 | 212 | 301 |
| 2583 | 401 | 7 | 204 | 201 | 201 | 307 |
| 2584 | 401 | 35 | 202 | 202 | 212 | 301 |
| 2585 | 407 | 100 | 212 | 212 | 212 | 308 |
| 2586 | 401 | 98 | 205 | 201 | 201 | 302 |
| 2587 | 401 | 84 | 203 | 212 | 212 | 303 |
| 2588 | 404 | 96 | 202 | 201 | 201 | 302 |
| 2589 | 406 | 116 | 205 | 202 | 212 | 301 |
| 2590 | 407 | 31 | 208 | 212 | 212 | 305 |
| 2591 | 402 | 6 | 210 | 202 | 212 | 306 |
| 2592 | 401 | 96 | 204 | 201 | 201 | 308 |
| 2593 | 405 | 19 | 202 | 201 | 201 | 308 |
| 2594 | 407 | 75 | 204 | 202 | 212 | 302 |
| 2595 | 401 | 13 | 204 | 201 | 201 | 302 |
| 2596 | 405 | 96 | 201 | 201 | 201 | 304 |
| 2597 | 401 | 53 | 204 | 201 | 201 | 303 |
| 2598 | 401 | 96 | 212 | 201 | 201 | 301 |
| 2599 | 401 | 58 | 202 | 212 | 212 | 306 |
| 2600 | 401 | 86 | 212 | 212 | 212 | 301 |
| 2601 | 403 | 102 | 201 | 201 | 201 | 308 |
| 2602 | 406 | 129 | 211 | 202 | 212 | 302 |
| 2603 | 401 | 49 | 202 | 201 | 201 | 307 |
| 2604 | 401 | 85 | 204 | 202 | 212 | 301 |
| 2605 | 402 | 87 | 201 | 202 | 212 | 302 |
| 2606 | 401 | 85 | 212 | 212 | 212 | 307 |
| 2607 | 403 | 98 | 204 | 212 | 212 | 301 |
| 2608 | 401 | 117 | 211 | 202 | 212 | 302 |
| 2609 | 401 | 21 | 212 | 202 | 212 | 301 |
| 2610 | 402 | 109 | 207 | 201 | 201 | 306 |
| 2611 | 406 | 77 | 204 | 212 | 212 | 301 |
| 2612 | 401 | 40 | 210 | 202 | 212 | 306 |
| 2613 | 403 | 72 | 204 | 202 | 212 | 305 |
| 2614 | 402 | 38 | 201 | 201 | 201 | 305 |
| 2615 | 401 | 99 | 203 | 202 | 212 | 307 |
| 2616 | 401 | 89 | 201 | 202 | 212 | 306 |
| 2617 | 404 | 127 | 210 | 201 | 201 | 301 |
| 2618 | 403 | 93 | 205 | 212 | 212 | 307 |
| 2619 | 406 | 16 | 205 | 201 | 201 | 306 |
| 2620 | 406 | 122 | 201 | 201 | 201 | 308 |
| 2621 | 403 | 125 | 201 | 212 | 212 | 308 |
| 2622 | 401 | 96 | 211 | 201 | 201 | 301 |
| 2623 | 401 | 92 | 204 | 212 | 212 | 301 |
| 2624 | 401 | 104 | 210 | 202 | 212 | 307 |
| 2625 | 401 | 126 | 212 | 202 | 212 | 307 |
| 2626 | 403 | 101 | 207 | 212 | 212 | 301 |
| 2627 | 407 | 41 | 202 | 202 | 212 | 302 |
| 2628 | 401 | 123 | 201 | 201 | 201 | 307 |
| 2629 | 405 | 109 | 207 | 201 | 201 | 304 |
| 2630 | 404 | 8 | 204 | 212 | 212 | 301 |
| 2631 | 401 | 69 | 210 | 201 | 201 | 303 |
| 2632 | 402 | 11 | 212 | 212 | 212 | 306 |
| 2633 | 407 | 9 | 212 | 212 | 212 | 305 |
| 2634 | 402 | 32 | 211 | 212 | 212 | 301 |
| 2635 | 402 | 76 | 202 | 202 | 212 | 306 |
| 2636 | 407 | 42 | 205 | 212 | 212 | 308 |
| 2637 | 401 | 63 | 210 | 202 | 212 | 308 |
| 2638 | 401 | 120 | 201 | 201 | 201 | 304 |
| 2639 | 402 | 107 | 204 | 201 | 201 | 302 |
| 2640 | 406 | 90 | 205 | 212 | 212 | 305 |
| 2641 | 407 | 59 | 201 | 201 | 201 | 306 |
| 2642 | 402 | 68 | 204 | 212 | 212 | 304 |
| 2643 | 406 | 66 | 201 | 212 | 212 | 304 |
| 2644 | 401 | 15 | 211 | 212 | 212 | 302 |
| 2645 | 405 | 86 | 201 | 212 | 212 | 302 |
| 2646 | 401 | 113 | 210 | 201 | 201 | 307 |
| 2647 | 401 | 60 | 202 | 201 | 201 | 301 |
| 2648 | 402 | 70 | 205 | 201 | 201 | 302 |
| 2649 | 401 | 114 | 212 | 202 | 212 | 304 |
| 2650 | 401 | 26 | 202 | 212 | 212 | 303 |
| 2651 | 401 | 20 | 201 | 201 | 201 | 305 |
| 2652 | 406 | 91 | 210 | 201 | 201 | 308 |
| 2653 | 401 | 78 | 204 | 201 | 201 | 307 |
| 2654 | 406 | 54 | 212 | 201 | 201 | 302 |
| 2655 | 404 | 36 | 212 | 201 | 201 | 301 |
| 2656 | 407 | 12 | 207 | 201 | 201 | 303 |
| 2657 | 401 | 97 | 210 | 201 | 201 | 304 |
| 2658 | 402 | 83 | 211 | 212 | 212 | 303 |
| 2659 | 406 | 128 | 212 | 201 | 201 | 303 |
| 2660 | 401 | 56 | 202 | 202 | 212 | 304 |
| 2661 | 406 | 109 | 204 | 201 | 201 | 303 |
| 2662 | 405 | 111 | 204 | 202 | 212 | 308 |
| 2663 | 405 | 24 | 204 | 201 | 201 | 301 |
| 2664 | 401 | 88 | 202 | 212 | 212 | 307 |
| 2665 | 401 | 109 | 207 | 201 | 201 | 302 |
| 2666 | 407 | 86 | 210 | 212 | 212 | 303 |
| 2667 | 401 | 33 | 203 | 202 | 212 | 302 |
| 2668 | 407 | 98 | 202 | 201 | 201 | 307 |
| 2669 | 401 | 85 | 210 | 202 | 212 | 303 |
| 2670 | 406 | 45 | 205 | 201 | 201 | 303 |
| 2671 | 401 | 130 | 201 | 202 | 212 | 306 |
| 2672 | 401 | 2 | 201 | 201 | 201 | 308 |
| 2673 | 407 | 119 | 212 | 212 | 212 | 302 |
| 2674 | 401 | 39 | 204 | 212 | 212 | 302 |
| 2675 | 407 | 57 | 202 | 212 | 212 | 308 |
| 2676 | 401 | 10 | 211 | 202 | 212 | 308 |
| 2677 | 404 | 85 | 204 | 212 | 212 | 308 |
| 2678 | 407 | 98 | 208 | 201 | 201 | 308 |
| 2679 | 406 | 96 | 201 | 201 | 201 | 308 |

Compounds having the formula:

TABLE 16

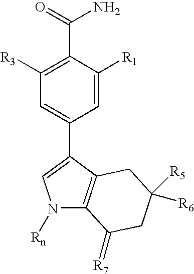

wherein R₁, R₃, Rₙ, R₅, R₆, and R₇ are defined in Table 16:

| Compound No. | R₁ | R₃ | Rₙ | R₅ | R₆ | R₇ |
|---|---|---|---|---|---|---|
| 2680 | 401 | 67 | 211 | 201 | 201 | 302 |
| 2681 | 405 | 115 | 201 | 201 | 201 | 308 |
| 2682 | 403 | 101 | 207 | 212 | 212 | 301 |
| 2683 | 401 | 89 | 201 | 202 | 212 | 306 |
| 2684 | 401 | 65 | 212 | 212 | 212 | 306 |
| 2685 | 401 | 88 | 201 | 202 | 212 | 303 |
| 2686 | 401 | 48 | 212 | 202 | 212 | 303 |
| 2687 | 404 | 127 | 210 | 201 | 201 | 301 |
| 2688 | 407 | 57 | 202 | 212 | 212 | 308 |
| 2689 | 401 | 104 | 210 | 202 | 212 | 307 |
| 2690 | 402 | 109 | 207 | 201 | 201 | 306 |
| 2691 | 404 | 18 | 202 | 202 | 212 | 306 |
| 2692 | 407 | 98 | 202 | 201 | 201 | 307 |
| 2693 | 403 | 88 | 211 | 201 | 201 | 306 |
| 2694 | 404 | 47 | 203 | 202 | 212 | 306 |
| 2695 | 401 | 17 | 207 | 201 | 201 | 308 |
| 2696 | 405 | 109 | 207 | 201 | 201 | 304 |
| 2697 | 401 | 112 | 212 | 201 | 201 | 304 |
| 2698 | 405 | 101 | 203 | 201 | 201 | 301 |
| 2699 | 401 | 98 | 212 | 201 | 201 | 307 |
| 2700 | 401 | 13 | 204 | 201 | 201 | 302 |
| 2701 | 401 | 40 | 210 | 202 | 212 | 306 |
| 2702 | 404 | 91 | 202 | 212 | 212 | 302 |
| 2703 | 404 | 88 | 210 | 202 | 212 | 306 |
| 2704 | 402 | 27 | 212 | 202 | 212 | 306 |
| 2705 | 401 | 49 | 202 | 201 | 201 | 307 |
| 2706 | 401 | 91 | 212 | 201 | 201 | 304 |
| 2707 | 401 | 99 | 203 | 202 | 212 | 307 |
| 2708 | 401 | 52 | 201 | 202 | 212 | 306 |
| 2709 | 402 | 83 | 211 | 212 | 212 | 303 |
| 2710 | 402 | 38 | 201 | 201 | 201 | 305 |
| 2711 | 407 | 86 | 210 | 212 | 212 | 303 |
| 2712 | 403 | 3 | 203 | 202 | 212 | 302 |
| 2713 | 403 | 101 | 211 | 201 | 201 | 306 |
| 2714 | 401 | 1 | 201 | 212 | 212 | 308 |
| 2715 | 403 | 93 | 205 | 212 | 212 | 307 |
| 2716 | 405 | 74 | 210 | 201 | 201 | 303 |
| 2717 | 402 | 106 | 205 | 212 | 212 | 307 |
| 2718 | 405 | 111 | 204 | 202 | 212 | 308 |
| 2719 | 401 | 28 | 212 | 202 | 212 | 302 |
| 2720 | 404 | 36 | 212 | 201 | 201 | 301 |
| 2721 | 404 | 85 | 204 | 212 | 212 | 308 |
| 2722 | 401 | 26 | 202 | 212 | 212 | 303 |
| 2723 | 404 | 25 | 204 | 202 | 212 | 306 |
| 2724 | 401 | 80 | 204 | 212 | 212 | 306 |
| 2725 | 407 | 43 | 212 | 202 | 212 | 308 |
| 2726 | 402 | 124 | 203 | 201 | 201 | 308 |
| 2727 | 406 | 116 | 205 | 202 | 212 | 301 |
| 2728 | 401 | 91 | 201 | 212 | 212 | 307 |
| 2729 | 401 | 56 | 202 | 202 | 212 | 304 |
| 2730 | 404 | 86 | 201 | 202 | 212 | 301 |
| 2731 | 401 | 22 | 211 | 201 | 201 | 308 |
| 2732 | 401 | 110 | 207 | 201 | 201 | 302 |
| 2733 | 402 | 109 | 201 | 212 | 212 | 301 |
| 2734 | 404 | 88 | 210 | 201 | 201 | 301 |
| 2735 | 405 | 79 | 201 | 212 | 212 | 301 |
| 2736 | 406 | 90 | 205 | 212 | 212 | 305 |
| 2737 | 403 | 125 | 201 | 212 | 212 | 308 |
| 2738 | 406 | 91 | 210 | 201 | 201 | 308 |
| 2739 | 401 | 23 | 205 | 202 | 212 | 301 |
| 2740 | 401 | 120 | 201 | 201 | 201 | 304 |
| 2741 | 401 | 96 | 212 | 201 | 201 | 301 |
| 2742 | 401 | 96 | 204 | 201 | 201 | 308 |
| 2743 | 401 | 88 | 202 | 212 | 212 | 307 |
| 2744 | 407 | 42 | 205 | 212 | 212 | 308 |
| 2745 | 406 | 109 | 204 | 212 | 212 | 303 |
| 2746 | 403 | 46 | 204 | 202 | 212 | 308 |
| 2747 | 402 | 76 | 202 | 202 | 212 | 306 |
| 2748 | 403 | 98 | 204 | 212 | 212 | 301 |
| 2749 | 402 | 32 | 211 | 212 | 212 | 301 |
| 2750 | 407 | 109 | 212 | 201 | 201 | 305 |
| 2751 | 405 | 19 | 202 | 201 | 201 | 308 |
| 2752 | 401 | 34 | 205 | 201 | 201 | 304 |
| 2753 | 406 | 122 | 201 | 201 | 201 | 308 |
| 2754 | 403 | 72 | 204 | 202 | 212 | 305 |
| 2755 | 406 | 129 | 211 | 202 | 212 | 302 |
| 2756 | 406 | 66 | 201 | 212 | 212 | 304 |
| 2757 | 404 | 96 | 212 | 212 | 212 | 307 |
| 2758 | 407 | 12 | 207 | 201 | 201 | 303 |
| 2759 | 403 | 101 | 212 | 202 | 212 | 307 |
| 2760 | 405 | 86 | 205 | 201 | 201 | 303 |
| 2761 | 401 | 85 | 204 | 202 | 212 | 301 |
| 2762 | 402 | 107 | 204 | 201 | 201 | 302 |
| 2763 | 402 | 68 | 204 | 212 | 212 | 304 |
| 2764 | 401 | 2 | 201 | 201 | 201 | 308 |
| 2765 | 401 | 60 | 202 | 201 | 201 | 301 |
| 2766 | 402 | 108 | 205 | 212 | 212 | 302 |
| 2767 | 406 | 128 | 212 | 201 | 201 | 303 |
| 2768 | 401 | 86 | 212 | 212 | 212 | 301 |
| 2769 | 404 | 81 | 210 | 212 | 212 | 301 |
| 2770 | 401 | 63 | 210 | 202 | 212 | 308 |
| 2771 | 401 | 118 | 203 | 212 | 212 | 306 |
| 2772 | 406 | 94 | 201 | 212 | 212 | 304 |
| 2773 | 402 | 6 | 210 | 202 | 212 | 306 |
| 2774 | 401 | 53 | 204 | 201 | 201 | 303 |
| 2775 | 402 | 44 | 202 | 201 | 201 | 307 |
| 2776 | 401 | 39 | 204 | 212 | 212 | 302 |
| 2777 | 401 | 82 | 202 | 202 | 212 | 302 |
| 2778 | 403 | 102 | 201 | 201 | 201 | 308 |
| 2779 | 407 | 75 | 204 | 202 | 212 | 302 |
| 2780 | 401 | 130 | 201 | 202 | 212 | 306 |
| 2781 | 405 | 85 | 211 | 212 | 212 | 301 |
| 2782 | 402 | 11 | 212 | 212 | 212 | 306 |
| 2783 | 401 | 109 | 207 | 201 | 201 | 302 |
| 2784 | 401 | 29 | 210 | 202 | 212 | 306 |
| 2785 | 406 | 77 | 204 | 212 | 212 | 301 |
| 2786 | 404 | 86 | 204 | 212 | 212 | 304 |
| 2787 | 401 | 69 | 210 | 201 | 201 | 303 |
| 2788 | 407 | 41 | 202 | 202 | 212 | 302 |
| 2789 | 406 | 109 | 204 | 201 | 201 | 303 |
| 2790 | 407 | 119 | 212 | 212 | 212 | 302 |
| 2791 | 401 | 15 | 211 | 212 | 212 | 302 |
| 2792 | 402 | 88 | 201 | 212 | 212 | 301 |
| 2793 | 405 | 71 | 211 | 202 | 212 | 306 |
| 2794 | 407 | 9 | 212 | 212 | 212 | 305 |
| 2795 | 406 | 45 | 205 | 201 | 201 | 303 |
| 2796 | 401 | 50 | 212 | 202 | 212 | 307 |
| 2797 | 402 | 87 | 201 | 202 | 212 | 302 |
| 2798 | 405 | 96 | 201 | 201 | 201 | 304 |
| 2799 | 406 | 4 | 201 | 201 | 201 | 304 |
| 2800 | 407 | 98 | 208 | 201 | 201 | 308 |
| 2801 | 405 | 86 | 201 | 212 | 212 | 302 |
| 2802 | 401 | 96 | 211 | 201 | 201 | 301 |
| 2803 | 401 | 51 | 211 | 201 | 201 | 307 |
| 2804 | 401 | 73 | 210 | 202 | 212 | 308 |
| 2805 | 407 | 31 | 208 | 212 | 212 | 305 |
| 2806 | 401 | 33 | 203 | 202 | 212 | 302 |
| 2807 | 406 | 16 | 205 | 201 | 201 | 306 |
| 2808 | 402 | 70 | 205 | 201 | 201 | 302 |
| 2809 | 401 | 101 | 201 | 212 | 212 | 308 |
| 2810 | 405 | 85 | 204 | 212 | 212 | 301 |
| 2811 | 405 | 64 | 212 | 201 | 201 | 308 |
| 2812 | 401 | 21 | 212 | 202 | 212 | 301 |
| 2813 | 405 | 91 | 203 | 201 | 201 | 306 |
| 2814 | 403 | 88 | 204 | 202 | 212 | 306 |
| 2815 | 401 | 55 | 204 | 201 | 201 | 302 |
| 2816 | 403 | 61 | 202 | 202 | 212 | 301 |
| 2817 | 401 | 58 | 202 | 212 | 212 | 306 |
| 2818 | 401 | 10 | 211 | 202 | 212 | 308 |
| 2819 | 401 | 121 | 202 | 202 | 212 | 308 |
| 2820 | 401 | 35 | 202 | 202 | 212 | 301 |
| 2821 | 401 | 97 | 210 | 201 | 201 | 304 |
| 2822 | 401 | 78 | 204 | 201 | 201 | 307 |
| 2823 | 401 | 85 | 210 | 202 | 212 | 303 |
| 2824 | 401 | 88 | 208 | 212 | 212 | 301 |
| 2825 | 405 | 105 | 212 | 202 | 212 | 306 |
| 2826 | 401 | 7 | 204 | 201 | 201 | 307 |
| 2827 | 401 | 84 | 203 | 212 | 212 | 303 |
| 2828 | 406 | 54 | 212 | 201 | 201 | 302 |
| 2829 | 407 | 59 | 201 | 201 | 201 | 306 |
| 2830 | 407 | 100 | 212 | 212 | 212 | 308 |
| 2831 | 407 | 5 | 205 | 201 | 201 | 302 |
| 2832 | 401 | 126 | 212 | 202 | 212 | 307 |
| 2833 | 406 | 96 | 201 | 201 | 201 | 308 |
| 2834 | 407 | 91 | 201 | 201 | 201 | 305 |
| 2835 | 401 | 113 | 210 | 201 | 201 | 307 |
| 2836 | 405 | 24 | 204 | 201 | 201 | 301 |
| 2837 | 401 | 98 | 208 | 201 | 201 | 301 |
| 2838 | 401 | 117 | 211 | 202 | 212 | 302 |
| 2839 | 404 | 96 | 210 | 212 | 212 | 305 |
| 2840 | 401 | 98 | 205 | 201 | 201 | 302 |
| 2841 | 401 | 85 | 212 | 212 | 212 | 307 |
| 2842 | 403 | 62 | 205 | 212 | 212 | 303 |
| 2843 | 404 | 37 | 204 | 202 | 212 | 306 |
| 2844 | 406 | 103 | 205 | 201 | 201 | 301 |
| 2845 | 403 | 101 | 205 | 202 | 212 | 302 |
| 2846 | 404 | 96 | 202 | 201 | 201 | 302 |
| 2847 | 401 | 123 | 201 | 201 | 201 | 307 |
| 2848 | 403 | 30 | 210 | 201 | 201 | 305 |
| 2849 | 401 | 114 | 212 | 202 | 212 | 304 |
| 2850 | 404 | 8 | 204 | 212 | 212 | 301 |
| 2851 | 401 | 20 | 201 | 201 | 201 | 305 |
| 2852 | 401 | 92 | 204 | 212 | 212 | 301 |
| 2853 | 403 | 14 | 203 | 201 | 201 | 308 |

Compounds having the formula:

TABLE 17

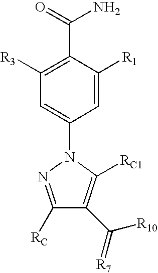

wherein R₁, R₃, R_{C}, R_{C1}, R₁₀, and R₇ are listed in Table 17.

| Compound No. | R₁ | R₃ | R_{C} | R_{C1} | R₁₀ | R₇ |
|---|---|---|---|---|---|---|
| 2854 | 401 | 58 | 202 | 212 | 211 | 306 |
| 2855 | 406 | 96 | 201 | 201 | 201 | 308 |
| 2856 | 401 | 30 | 210 | 201 | 201 | 305 |
| 2857 | 405 | 98 | 204 | 202 | 208 | 301 |
| 2858 | 401 | 91 | 206 | 202 | 207 | 307 |
| 2859 | 401 | 80 | 204 | 212 | 206 | 306 |
| 2860 | 401 | 122 | 201 | 201 | 201 | 308 |
| 2861 | 404 | 42 | 205 | 212 | 207 | 308 |
| 2862 | 407 | 101 | 212 | 202 | 207 | 307 |
| 2863 | 403 | 101 | 211 | 201 | 201 | 306 |
| 2864 | 403 | 70 | 205 | 201 | 207 | 302 |
| 2865 | 401 | 109 | 201 | 212 | 208 | 301 |
| 2866 | 401 | 15 | 211 | 202 | 201 | 302 |
| 2867 | 405 | 88 | 210 | 201 | 201 | 301 |
| 2868 | 402 | 96 | 201 | 201 | 201 | 304 |
| 2869 | 401 | 98 | 212 | 201 | 201 | 307 |
| 2870 | 405 | 46 | 204 | 202 | 208 | 308 |
| 2871 | 401 | 90 | 205 | 202 | 201 | 305 |
| 2872 | 401 | 1 | 201 | 201 | 206 | 308 |
| 2873 | 403 | 25 | 204 | 202 | 201 | 306 |
| 2874 | 401 | 81 | 210 | 202 | 208 | 301 |
| 2875 | 405 | 104 | 210 | 202 | 204 | 307 |
| 2876 | 401 | 27 | 212 | 202 | 203 | 306 |
| 2877 | 404 | 12 | 207 | 201 | 201 | 303 |
| 2878 | 403 | 52 | 201 | 202 | 207 | 306 |
| 2879 | 401 | 65 | 212 | 212 | 202 | 306 |
| 2880 | 406 | 88 | 208 | 212 | 211 | 301 |
| 2881 | 402 | 109 | 212 | 201 | 201 | 305 |
| 2882 | 404 | 4 | 201 | 201 | 201 | 304 |
| 2883 | 401 | 57 | 202 | 212 | 208 | 308 |
| 2884 | 404 | 96 | 212 | 201 | 201 | 301 |
| 2885 | 402 | 97 | 210 | 201 | 201 | 304 |
| 2886 | 405 | 76 | 202 | 202 | 201 | 306 |
| 2887 | 407 | 3 | 203 | 202 | 201 | 302 |
| 2888 | 404 | 89 | 206 | 202 | 204 | 306 |
| 2889 | 404 | 28 | 212 | 202 | 209 | 302 |
| 2890 | 405 | 55 | 204 | 201 | 201 | 302 |
| 2891 | 403 | 92 | 204 | 202 | 209 | 301 |
| 2892 | 404 | 85 | 204 | 202 | 207 | 301 |
| 2893 | 401 | 85 | 210 | 202 | 211 | 303 |
| 2894 | 401 | 98 | 208 | 201 | 201 | 301 |
| 2895 | 405 | 88 | 204 | 202 | 208 | 306 |
| 2896 | 403 | 51 | 211 | 201 | 201 | 307 |
| 2897 | 405 | 47 | 203 | 202 | 203 | 306 |
| 2898 | 401 | 86 | 201 | 202 | 201 | 301 |
| 2899 | 402 | 33 | 203 | 202 | 209 | 302 |
| 2900 | 404 | 44 | 202 | 201 | 201 | 307 |
| 2901 | 401 | 40 | 210 | 202 | 203 | 306 |
| 2902 | 402 | 94 | 201 | 212 | 205 | 304 |
| 2903 | 401 | 41 | 202 | 202 | 201 | 302 |
| 2904 | 403 | 109 | 207 | 201 | 201 | 302 |
| 2905 | 401 | 127 | 210 | 201 | 201 | 301 |
| 2906 | 401 | 31 | 208 | 212 | 211 | 305 |
| 2907 | 401 | 45 | 205 | 201 | 201 | 303 |
| 2908 | 401 | 110 | 207 | 201 | 201 | 302 |
| 2909 | 405 | 103 | 205 | 201 | 201 | 301 |
| 2910 | 401 | 22 | 211 | 201 | 201 | 308 |
| 2911 | 403 | 96 | 209 | 201 | 201 | 302 |
| 2912 | 404 | 56 | 209 | 202 | 209 | 304 |
| 2913 | 402 | 79 | 206 | 201 | 207 | 301 |
| 2914 | 401 | 93 | 205 | 212 | 202 | 307 |
| 2915 | 405 | 35 | 209 | 202 | 201 | 301 |
| 2916 | 401 | 13 | 204 | 201 | 201 | 302 |
| 2917 | 406 | 128 | 212 | 201 | 201 | 303 |
| 2918 | 401 | 86 | 204 | 212 | 204 | 304 |
| 2919 | 401 | 115 | 206 | 201 | 201 | 308 |
| 2920 | 404 | 86 | 201 | 212 | 209 | 302 |
| 2921 | 405 | 85 | 211 | 202 | 201 | 301 |
| 2922 | 403 | 88 | 211 | 201 | 201 | 306 |
| 2923 | 401 | 101 | 203 | 201 | 201 | 301 |
| 2924 | 407 | 61 | 202 | 202 | 209 | 301 |
| 2925 | 402 | 5 | 205 | 201 | 201 | 302 |
| 2926 | 403 | 95 | 204 | 201 | 201 | 308 |
| 2927 | 404 | 100 | 212 | 202 | 202 | 308 |
| 2928 | 406 | 38 | 206 | 201 | 201 | 305 |
| 2929 | 407 | 88 | 209 | 201 | 209 | 307 |
| 2930 | 403 | 118 | 203 | 202 | 209 | 306 |
| 2931 | 404 | 113 | 210 | 201 | 201 | 307 |
| 2932 | 401 | 43 | 212 | 202 | 201 | 308 |
| 2933 | 401 | 73 | 210 | 202 | 209 | 308 |
| 2934 | 407 | 34 | 205 | 201 | 201 | 304 |
| 2935 | 401 | 69 | 210 | 201 | 201 | 303 |
| 2936 | 401 | 91 | 209 | 212 | 201 | 302 |
| 2937 | 404 | 91 | 210 | 201 | 201 | 308 |
| 2938 | 406 | 83 | 211 | 212 | 206 | 303 |
| 2939 | 407 | 109 | 204 | 212 | 207 | 303 |
| 2940 | 402 | 19 | 202 | 201 | 201 | 308 |
| 2941 | 401 | 67 | 211 | 201 | 201 | 302 |
| 2942 | 405 | 96 | 204 | 201 | 201 | 308 |
| 2943 | 407 | 107 | 204 | 201 | 201 | 302 |
| 2944 | 401 | 72 | 204 | 202 | 201 | 305 |
| 2945 | 405 | 10 | 211 | 202 | 201 | 308 |
| 2946 | 401 | 88 | 206 | 202 | 203 | 303 |
| 2947 | 401 | 78 | 204 | 201 | 201 | 307 |
| 2948 | 401 | 68 | 204 | 201 | 201 | 304 |
| 2949 | 401 | 124 | 203 | 201 | 201 | 308 |
| 2950 | 403 | 88 | 210 | 202 | 204 | 306 |
| 2951 | 406 | 49 | 209 | 201 | 201 | 307 |
| 2952 | 401 | 96 | 212 | 202 | 204 | 307 |
| 2953 | 401 | 119 | 212 | 212 | 208 | 302 |
| 2954 | 402 | 74 | 210 | 201 | 201 | 303 |
| 2955 | 401 | 105 | 212 | 202 | 208 | 306 |
| 2956 | 403 | 85 | 212 | 212 | 201 | 307 |
| 2957 | 401 | 21 | 212 | 202 | 208 | 301 |
| 2958 | 401 | 63 | 210 | 202 | 211 | 308 |
| 2959 | 402 | 126 | 212 | 202 | 202 | 307 |
| 2960 | 406 | 36 | 212 | 201 | 201 | 301 |
| 2961 | 401 | 87 | 206 | 202 | 209 | 302 |
| 2962 | 403 | 59 | 206 | 201 | 201 | 306 |
| 2963 | 402 | 112 | 212 | 201 | 201 | 304 |
| 2964 | 401 | 48 | 212 | 202 | 207 | 303 |
| 2965 | 401 | 120 | 201 | 201 | 201 | 304 |
| 2966 | 404 | 14 | 203 | 201 | 201 | 308 |
| 2967 | 403 | 121 | 209 | 202 | 206 | 308 |
| 2968 | 406 | 39 | 204 | 212 | 208 | 302 |
| 2969 | 406 | 96 | 210 | 212 | 201 | 305 |
| 2970 | 403 | 53 | 204 | 201 | 201 | 303 |
| 2971 | 401 | 16 | 205 | 201 | 201 | 306 |
| 2972 | 401 | 102 | 201 | 201 | 201 | 308 |
| 2973 | 401 | 23 | 205 | 202 | 209 | 301 |
| 2974 | 401 | 85 | 204 | 212 | 209 | 301 |
| 2975 | 403 | 71 | 211 | 202 | 203 | 306 |
| 2976 | 407 | 54 | 212 | 201 | 201 | 302 |
| 2977 | 401 | 91 | 203 | 201 | 201 | 306 |
| 2978 | 405 | 32 | 211 | 212 | 201 | 301 |
| 2979 | 404 | 116 | 205 | 202 | 208 | 301 |
| 2980 | 401 | 77 | 204 | 212 | 204 | 301 |
| 2981 | 402 | 99 | 203 | 202 | 207 | 307 |
| 2982 | 407 | 88 | 206 | 201 | 201 | 301 |
| 2983 | 402 | 9 | 212 | 212 | 208 | 305 |
| 2984 | 402 | 64 | 212 | 201 | 201 | 308 |
| 2985 | 407 | 60 | 202 | 201 | 201 | 301 |
| 2986 | 401 | 37 | 204 | 202 | 209 | 306 |
| 2987 | 401 | 101 | 201 | 212 | 201 | 308 |
| 2988 | 402 | 86 | 205 | 201 | 201 | 303 |
| 2989 | 406 | 62 | 205 | 212 | 209 | 303 |
| 2990 | 407 | 109 | 204 | 201 | 201 | 303 |
| 2991 | 402 | 106 | 205 | 201 | 208 | 307 |
| 2992 | 406 | 84 | 203 | 201 | 207 | 303 |
| 2993 | 401 | 17 | 207 | 201 | 201 | 308 |
| 2994 | 405 | 86 | 212 | 212 | 203 | 301 |
| 2995 | 401 | 117 | 211 | 202 | 201 | 302 |
| 2996 | 401 | 123 | 201 | 201 | 201 | 307 |
| 2997 | 402 | 101 | 205 | 202 | 211 | 302 |
| 2998 | 401 | 125 | 201 | 212 | 202 | 308 |
| 2999 | 401 | 26 | 209 | 212 | 201 | 303 |
| 3000 | 401 | 96 | 211 | 201 | 201 | 301 |
| 3001 | 406 | 75 | 204 | 202 | 201 | 302 |
| 3002 | 401 | 50 | 212 | 202 | 201 | 307 |
| 3003 | 406 | 29 | 210 | 202 | 205 | 306 |
| 3004 | 404 | 111 | 204 | 202 | 207 | 308 |
| 3005 | 407 | 85 | 204 | 201 | 204 | 308 |
| 3006 | 401 | 98 | 209 | 201 | 201 | 307 |
| 3007 | 402 | 24 | 204 | 201 | 201 | 301 |
| 3008 | 406 | 6 | 210 | 202 | 202 | 306 |
| 3009 | 401 | 11 | 212 | 201 | 206 | 306 |
| 3010 | 406 | 130 | 206 | 202 | 208 | 306 |
| 3011 | 405 | 8 | 204 | 212 | 201 | 301 |
| 3012 | 405 | 18 | 202 | 202 | 207 | 306 |
| 3013 | 401 | 86 | 210 | 212 | 201 | 303 |
| 3014 | 401 | 108 | 205 | 201 | 204 | 302 |
| 3015 | 407 | 98 | 205 | 201 | 201 | 302 |
| 3016 | 401 | 82 | 209 | 202 | 201 | 302 |
| 3017 | 407 | 2 | 201 | 201 | 201 | 308 |
| 3018 | 401 | 20 | 201 | 201 | 201 | 305 |
| 3019 | 406 | 91 | 212 | 201 | 201 | 304 |
| 3020 | 401 | 129 | 211 | 202 | 201 | 302 |
| 3021 | 401 | 98 | 208 | 201 | 201 | 308 |
| 3022 | 407 | 7 | 204 | 201 | 201 | 307 |
| 3023 | 401 | 109 | 207 | 201 | 201 | 304 |
| 3024 | 407 | 109 | 207 | 201 | 201 | 306 |
| 3025 | 401 | 91 | 201 | 201 | 201 | 305 |
| 3026 | 404 | 114 | 212 | 202 | 206 | 304 |
| 3027 | 407 | 101 | 207 | 201 | 202 | 301 |
| 3028 | 406 | 66 | 201 | 201 | 208 | 304 |

Compounds having the formula:

TABLE 18

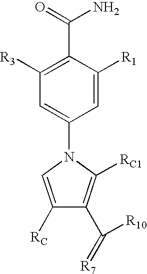

wherein R₁, R₃, R_{C}, R_{C1}, R₁₀, and R₇ are listed in Table 18.

| Compound | | | | | | |
|---|---|---|---|---|---|---|
| No. | R₁ | R₃ | R_{C} | R_{C1} | R₁₀ | R₇ |
| 3029 | 401 | 58 | 202 | 212 | 211 | 306 |
| 3030 | 406 | 96 | 201 | 201 | 201 | 308 |
| 3031 | 401 | 30 | 210 | 201 | 201 | 305 |
| 3032 | 405 | 98 | 204 | 202 | 208 | 301 |
| 3033 | 401 | 91 | 206 | 202 | 207 | 307 |
| 3034 | 401 | 80 | 204 | 212 | 206 | 306 |
| 3035 | 401 | 122 | 201 | 201 | 201 | 308 |
| 3036 | 404 | 42 | 205 | 212 | 207 | 308 |
| 3037 | 407 | 101 | 212 | 202 | 207 | 307 |
| 3038 | 403 | 101 | 211 | 201 | 201 | 306 |
| 3039 | 403 | 70 | 205 | 201 | 207 | 302 |
| 3040 | 401 | 109 | 201 | 212 | 208 | 301 |
| 3041 | 401 | 15 | 211 | 202 | 201 | 302 |
| 3042 | 405 | 88 | 210 | 201 | 201 | 301 |
| 3043 | 402 | 96 | 201 | 201 | 201 | 304 |
| 3044 | 401 | 98 | 212 | 201 | 201 | 307 |
| 3045 | 405 | 46 | 204 | 202 | 208 | 308 |
| 3046 | 401 | 90 | 205 | 202 | 201 | 305 |
| 3047 | 401 | 1 | 201 | 201 | 206 | 308 |
| 3048 | 403 | 25 | 204 | 202 | 201 | 306 |
| 3049 | 401 | 81 | 210 | 202 | 208 | 301 |
| 3050 | 405 | 104 | 210 | 202 | 204 | 307 |
| 3051 | 401 | 27 | 212 | 202 | 203 | 306 |
| 3052 | 404 | 12 | 207 | 201 | 201 | 303 |
| 3053 | 403 | 52 | 201 | 202 | 207 | 306 |
| 3054 | 401 | 65 | 212 | 212 | 202 | 306 |
| 3055 | 406 | 88 | 208 | 212 | 211 | 301 |
| 3056 | 402 | 109 | 212 | 201 | 201 | 305 |
| 3057 | 404 | 4 | 201 | 201 | 201 | 304 |
| 3058 | 401 | 57 | 202 | 212 | 208 | 308 |
| 3059 | 404 | 96 | 212 | 201 | 201 | 301 |
| 3060 | 402 | 97 | 210 | 201 | 201 | 304 |
| 3061 | 405 | 76 | 202 | 202 | 201 | 306 |
| 3062 | 407 | 3 | 203 | 202 | 201 | 302 |
| 3063 | 404 | 89 | 206 | 202 | 204 | 306 |
| 3064 | 404 | 28 | 212 | 202 | 209 | 302 |
| 3065 | 405 | 55 | 204 | 201 | 201 | 302 |
| 3066 | 403 | 92 | 204 | 202 | 209 | 301 |
| 3067 | 404 | 85 | 204 | 202 | 207 | 301 |
| 3068 | 401 | 85 | 210 | 202 | 211 | 303 |
| 3069 | 401 | 98 | 208 | 201 | 201 | 301 |
| 3070 | 405 | 88 | 204 | 202 | 208 | 306 |
| 3071 | 403 | 51 | 211 | 201 | 201 | 307 |
| 3072 | 405 | 47 | 203 | 202 | 203 | 306 |
| 3073 | 401 | 86 | 201 | 202 | 201 | 301 |
| 3074 | 402 | 33 | 203 | 202 | 209 | 302 |
| 3075 | 404 | 44 | 202 | 201 | 201 | 307 |
| 3076 | 401 | 40 | 210 | 202 | 203 | 306 |
| 3077 | 402 | 94 | 201 | 212 | 205 | 304 |
| 3078 | 401 | 41 | 202 | 202 | 201 | 302 |
| 3079 | 403 | 109 | 207 | 201 | 201 | 302 |
| 3080 | 401 | 127 | 210 | 201 | 201 | 301 |
| 3081 | 401 | 31 | 208 | 212 | 211 | 305 |
| 3082 | 401 | 45 | 205 | 201 | 201 | 303 |
| 3083 | 401 | 110 | 207 | 201 | 201 | 302 |
| 3084 | 405 | 103 | 205 | 201 | 201 | 301 |
| 3085 | 401 | 22 | 211 | 201 | 201 | 308 |
| 3086 | 403 | 96 | 209 | 201 | 201 | 302 |
| 3087 | 404 | 56 | 209 | 202 | 209 | 304 |
| 3088 | 402 | 79 | 206 | 201 | 207 | 301 |
| 3089 | 401 | 93 | 205 | 212 | 202 | 307 |
| 3090 | 405 | 35 | 209 | 202 | 201 | 301 |
| 3091 | 401 | 13 | 204 | 201 | 201 | 302 |
| 3092 | 406 | 128 | 212 | 201 | 201 | 303 |
| 3093 | 401 | 86 | 204 | 212 | 204 | 304 |
| 3094 | 401 | 115 | 206 | 201 | 201 | 308 |
| 3095 | 404 | 86 | 201 | 212 | 209 | 302 |
| 3096 | 405 | 85 | 211 | 202 | 201 | 301 |
| 3097 | 403 | 88 | 211 | 201 | 201 | 306 |
| 3098 | 401 | 101 | 203 | 201 | 201 | 301 |
| 3099 | 407 | 61 | 202 | 202 | 209 | 301 |
| 3100 | 402 | 5 | 205 | 201 | 201 | 302 |
| 3101 | 403 | 95 | 204 | 201 | 201 | 308 |
| 3102 | 404 | 100 | 212 | 202 | 202 | 308 |
| 3103 | 406 | 38 | 206 | 201 | 201 | 305 |
| 3104 | 407 | 88 | 209 | 201 | 209 | 307 |
| 3105 | 403 | 118 | 203 | 202 | 209 | 306 |
| 3106 | 404 | 113 | 210 | 201 | 201 | 307 |
| 3107 | 401 | 43 | 212 | 202 | 201 | 308 |
| 3108 | 401 | 73 | 210 | 202 | 209 | 308 |
| 3109 | 407 | 34 | 205 | 201 | 201 | 304 |
| 3110 | 401 | 69 | 210 | 201 | 201 | 303 |
| 3111 | 401 | 91 | 209 | 212 | 201 | 302 |
| 3112 | 404 | 91 | 210 | 201 | 201 | 308 |
| 3113 | 406 | 83 | 211 | 212 | 206 | 303 |
| 3114 | 407 | 109 | 204 | 212 | 207 | 303 |
| 3115 | 402 | 19 | 202 | 201 | 201 | 308 |
| 3116 | 401 | 67 | 211 | 201 | 201 | 302 |
| 3117 | 405 | 96 | 204 | 201 | 201 | 308 |
| 3118 | 407 | 107 | 204 | 201 | 201 | 302 |
| 3119 | 401 | 72 | 204 | 202 | 201 | 305 |
| 3120 | 405 | 10 | 211 | 202 | 201 | 308 |
| 3121 | 401 | 88 | 206 | 202 | 203 | 303 |
| 3122 | 401 | 78 | 204 | 201 | 201 | 307 |
| 3123 | 401 | 68 | 204 | 201 | 201 | 304 |
| 3124 | 401 | 124 | 203 | 201 | 201 | 308 |
| 3125 | 403 | 88 | 210 | 202 | 204 | 306 |
| 3126 | 406 | 49 | 209 | 201 | 201 | 307 |
| 3127 | 401 | 96 | 212 | 202 | 204 | 307 |
| 3128 | 401 | 119 | 212 | 212 | 208 | 302 |
| 3129 | 402 | 74 | 210 | 201 | 201 | 303 |
| 3130 | 401 | 105 | 212 | 202 | 208 | 306 |
| 3131 | 403 | 85 | 212 | 212 | 201 | 307 |
| 3132 | 401 | 21 | 212 | 202 | 208 | 301 |
| 3133 | 401 | 63 | 210 | 202 | 211 | 308 |
| 3134 | 402 | 126 | 212 | 202 | 202 | 307 |
| 3135 | 406 | 36 | 212 | 201 | 201 | 301 |
| 3136 | 401 | 87 | 206 | 202 | 209 | 302 |
| 3137 | 403 | 59 | 206 | 201 | 201 | 306 |
| 3138 | 402 | 112 | 212 | 201 | 201 | 304 |
| 3139 | 401 | 48 | 212 | 202 | 207 | 303 |
| 3140 | 401 | 120 | 201 | 201 | 201 | 304 |
| 3141 | 404 | 14 | 203 | 201 | 201 | 308 |
| 3142 | 403 | 121 | 209 | 202 | 206 | 308 |
| 3143 | 406 | 39 | 204 | 212 | 208 | 302 |
| 3144 | 406 | 96 | 210 | 212 | 201 | 305 |
| 3145 | 403 | 53 | 204 | 201 | 201 | 303 |
| 3146 | 401 | 16 | 205 | 201 | 201 | 306 |
| 3147 | 401 | 102 | 201 | 201 | 201 | 308 |
| 3148 | 401 | 23 | 205 | 202 | 209 | 301 |
| 3149 | 401 | 85 | 204 | 212 | 209 | 301 |
| 3150 | 403 | 71 | 211 | 202 | 203 | 306 |
| 3151 | 407 | 54 | 212 | 201 | 201 | 302 |
| 3152 | 401 | 91 | 203 | 201 | 201 | 306 |
| 3153 | 405 | 32 | 211 | 212 | 201 | 301 |
| 3154 | 404 | 116 | 205 | 202 | 208 | 301 |
| 3155 | 401 | 77 | 204 | 212 | 204 | 301 |
| 3156 | 402 | 99 | 203 | 202 | 207 | 307 |
| 3157 | 407 | 88 | 206 | 201 | 201 | 301 |
| 3158 | 402 | 9 | 212 | 212 | 208 | 305 |
| 3159 | 402 | 64 | 212 | 201 | 201 | 308 |
| 3160 | 407 | 60 | 202 | 201 | 201 | 301 |
| 3161 | 401 | 37 | 204 | 202 | 209 | 306 |
| 3162 | 401 | 101 | 201 | 212 | 201 | 308 |
| 3163 | 402 | 86 | 205 | 201 | 201 | 303 |
| 3164 | 406 | 62 | 205 | 212 | 209 | 303 |
| 3165 | 407 | 109 | 204 | 201 | 201 | 303 |
| 3166 | 402 | 106 | 205 | 201 | 208 | 307 |
| 3167 | 406 | 84 | 203 | 201 | 207 | 303 |
| 3168 | 401 | 17 | 207 | 201 | 201 | 308 |
| 3169 | 405 | 86 | 212 | 212 | 203 | 301 |
| 3170 | 401 | 117 | 211 | 202 | 201 | 302 |
| 3171 | 401 | 123 | 201 | 201 | 201 | 307 |
| 3172 | 402 | 101 | 205 | 202 | 211 | 302 |
| 3173 | 401 | 125 | 201 | 212 | 202 | 308 |
| 3174 | 401 | 26 | 209 | 212 | 201 | 303 |
| 3175 | 401 | 96 | 211 | 201 | 201 | 301 |
| 3176 | 406 | 75 | 204 | 202 | 201 | 302 |
| 3177 | 401 | 50 | 212 | 202 | 201 | 307 |
| 3178 | 406 | 29 | 210 | 202 | 205 | 306 |
| 3179 | 404 | 111 | 204 | 202 | 207 | 308 |
| 3180 | 407 | 85 | 204 | 201 | 204 | 308 |
| 3181 | 401 | 98 | 209 | 201 | 201 | 307 |
| 3182 | 402 | 24 | 204 | 201 | 201 | 301 |
| 3183 | 406 | 6 | 210 | 202 | 202 | 306 |
| 3184 | 401 | 11 | 212 | 201 | 206 | 306 |
| 3185 | 406 | 130 | 206 | 202 | 208 | 306 |
| 3186 | 405 | 8 | 204 | 212 | 201 | 301 |
| 3187 | 405 | 18 | 202 | 202 | 207 | 306 |
| 3188 | 401 | 86 | 210 | 212 | 201 | 303 |
| 3189 | 401 | 108 | 205 | 201 | 204 | 302 |
| 3190 | 407 | 98 | 205 | 201 | 201 | 302 |
| 3191 | 401 | 82 | 209 | 202 | 201 | 302 |
| 3192 | 407 | 2 | 201 | 201 | 201 | 308 |
| 3193 | 401 | 20 | 201 | 201 | 201 | 305 |
| 3194 | 406 | 91 | 212 | 201 | 201 | 304 |
| 3195 | 401 | 129 | 211 | 202 | 201 | 302 |
| 3196 | 401 | 98 | 208 | 201 | 201 | 308 |
| 3197 | 407 | 7 | 204 | 201 | 201 | 307 |
| 3198 | 401 | 109 | 207 | 201 | 201 | 304 |
| 3199 | 407 | 109 | 207 | 201 | 201 | 306 |
| 3200 | 401 | 91 | 201 | 201 | 201 | 305 |
| 3201 | 404 | 114 | 212 | 202 | 206 | 304 |
| 3202 | 407 | 101 | 207 | 201 | 202 | 301 |
| 3203 | 406 | 66 | 201 | 201 | 208 | 304 |

Compounds having the formula:

TABLE 19

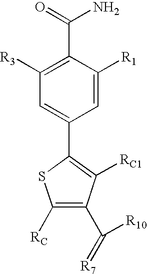

wherein R₁, R₃, R_{C}, R_{C1}, R₁₀, and R₇ are listed in Table 19.

| Compound | | | | | | |
|---|---|---|---|---|---|---|
| No. | R₁ | R₃ | R_{C} | R_{C1} | R₁₀ | R₇ |
| 3204 | 401 | 58 | 202 | 212 | 211 | 306 |
| 3205 | 406 | 96 | 201 | 201 | 201 | 308 |
| 3206 | 401 | 30 | 210 | 201 | 201 | 305 |
| 3207 | 405 | 98 | 204 | 202 | 208 | 301 |
| 3208 | 401 | 91 | 206 | 202 | 207 | 307 |
| 3209 | 401 | 80 | 204 | 212 | 206 | 306 |
| 3210 | 401 | 122 | 201 | 201 | 201 | 308 |
| 3211 | 404 | 42 | 205 | 212 | 207 | 308 |
| 3212 | 407 | 101 | 212 | 202 | 207 | 307 |
| 3213 | 403 | 101 | 211 | 201 | 201 | 306 |
| 3214 | 403 | 70 | 205 | 201 | 207 | 302 |
| 3215 | 401 | 109 | 201 | 212 | 208 | 301 |
| 3216 | 401 | 15 | 211 | 202 | 201 | 302 |
| 3217 | 405 | 88 | 210 | 201 | 201 | 301 |
| 3218 | 402 | 96 | 201 | 201 | 201 | 304 |
| 3219 | 401 | 98 | 212 | 201 | 201 | 307 |
| 3220 | 405 | 46 | 204 | 202 | 208 | 308 |
| 3221 | 401 | 90 | 205 | 202 | 201 | 305 |
| 3222 | 401 | 1 | 201 | 201 | 206 | 308 |
| 3223 | 403 | 25 | 204 | 202 | 201 | 306 |
| 3224 | 401 | 81 | 210 | 202 | 208 | 301 |
| 3225 | 405 | 104 | 210 | 202 | 204 | 307 |
| 3226 | 401 | 27 | 212 | 202 | 203 | 306 |
| 3227 | 404 | 12 | 207 | 201 | 201 | 303 |
| 3228 | 403 | 52 | 201 | 202 | 207 | 306 |
| 3229 | 401 | 65 | 212 | 212 | 202 | 306 |
| 3230 | 406 | 88 | 208 | 212 | 211 | 301 |
| 3231 | 402 | 109 | 212 | 201 | 201 | 305 |
| 3232 | 404 | 4 | 201 | 201 | 201 | 304 |
| 3233 | 401 | 57 | 202 | 212 | 208 | 308 |
| 3234 | 404 | 96 | 212 | 201 | 201 | 301 |
| 3235 | 402 | 97 | 210 | 201 | 201 | 304 |
| 3236 | 405 | 76 | 202 | 202 | 201 | 306 |
| 3237 | 407 | 3 | 203 | 202 | 201 | 302 |
| 3238 | 404 | 89 | 206 | 202 | 204 | 306 |
| 3239 | 404 | 28 | 212 | 202 | 209 | 302 |
| 3240 | 405 | 55 | 204 | 201 | 201 | 302 |
| 3241 | 403 | 92 | 204 | 202 | 209 | 301 |
| 3242 | 404 | 85 | 204 | 202 | 207 | 301 |
| 3243 | 401 | 85 | 210 | 202 | 211 | 303 |
| 3244 | 401 | 98 | 208 | 201 | 201 | 301 |
| 3245 | 405 | 88 | 204 | 202 | 208 | 306 |
| 3246 | 403 | 51 | 211 | 201 | 201 | 307 |
| 3247 | 405 | 47 | 203 | 202 | 203 | 306 |
| 3248 | 401 | 86 | 201 | 202 | 201 | 301 |
| 3249 | 402 | 33 | 203 | 202 | 209 | 302 |
| 3250 | 404 | 44 | 202 | 201 | 201 | 307 |
| 3251 | 401 | 40 | 210 | 202 | 203 | 306 |
| 3252 | 402 | 94 | 201 | 212 | 205 | 304 |
| 3253 | 401 | 41 | 202 | 202 | 201 | 302 |
| 3254 | 403 | 109 | 207 | 201 | 201 | 302 |
| 3255 | 401 | 127 | 210 | 201 | 201 | 301 |
| 3256 | 401 | 31 | 208 | 212 | 211 | 305 |
| 3257 | 401 | 45 | 205 | 201 | 201 | 303 |
| 3258 | 401 | 110 | 207 | 201 | 201 | 302 |
| 3259 | 405 | 103 | 205 | 201 | 201 | 301 |
| 3260 | 401 | 22 | 211 | 201 | 201 | 308 |
| 3261 | 403 | 96 | 209 | 201 | 201 | 302 |
| 3262 | 404 | 56 | 209 | 202 | 209 | 304 |
| 3263 | 402 | 79 | 206 | 201 | 207 | 301 |
| 3264 | 401 | 93 | 205 | 212 | 202 | 307 |
| 3265 | 405 | 35 | 209 | 202 | 201 | 301 |
| 3266 | 401 | 13 | 204 | 201 | 201 | 302 |
| 3267 | 406 | 128 | 212 | 201 | 201 | 303 |
| 3268 | 401 | 86 | 204 | 212 | 204 | 304 |
| 3269 | 401 | 115 | 206 | 201 | 201 | 308 |
| 3270 | 404 | 86 | 201 | 212 | 209 | 302 |
| 3271 | 405 | 85 | 211 | 202 | 201 | 301 |
| 3272 | 403 | 88 | 211 | 201 | 201 | 306 |
| 3273 | 401 | 101 | 203 | 201 | 201 | 301 |
| 3274 | 407 | 61 | 202 | 202 | 209 | 301 |
| 3275 | 402 | 5 | 205 | 201 | 201 | 302 |
| 3276 | 403 | 95 | 204 | 201 | 201 | 308 |
| 3277 | 404 | 100 | 212 | 202 | 202 | 308 |
| 3278 | 406 | 38 | 206 | 201 | 201 | 305 |
| 3279 | 407 | 88 | 209 | 201 | 209 | 307 |
| 3280 | 403 | 118 | 203 | 202 | 209 | 306 |
| 3281 | 404 | 113 | 210 | 201 | 201 | 307 |
| 3282 | 401 | 43 | 212 | 202 | 201 | 308 |
| 3283 | 401 | 73 | 210 | 202 | 209 | 308 |
| 3284 | 407 | 34 | 205 | 201 | 201 | 304 |
| 3285 | 401 | 69 | 210 | 201 | 201 | 303 |
| 3286 | 401 | 91 | 209 | 212 | 201 | 302 |
| 3287 | 404 | 91 | 210 | 201 | 201 | 308 |
| 3288 | 406 | 83 | 211 | 212 | 206 | 303 |
| 3289 | 407 | 109 | 204 | 212 | 207 | 303 |
| 3290 | 402 | 19 | 202 | 201 | 201 | 308 |
| 3291 | 401 | 67 | 211 | 201 | 201 | 302 |
| 3292 | 405 | 96 | 204 | 201 | 201 | 308 |
| 3293 | 407 | 107 | 204 | 201 | 201 | 302 |
| 3294 | 401 | 72 | 204 | 202 | 201 | 305 |
| 3295 | 405 | 10 | 211 | 202 | 201 | 308 |
| 3296 | 401 | 88 | 206 | 202 | 203 | 303 |
| 3297 | 401 | 78 | 204 | 201 | 201 | 307 |
| 3298 | 401 | 68 | 204 | 201 | 201 | 304 |
| 3299 | 401 | 124 | 203 | 201 | 201 | 308 |
| 3300 | 403 | 88 | 210 | 202 | 204 | 306 |
| 3301 | 406 | 49 | 209 | 201 | 201 | 307 |
| 3302 | 401 | 96 | 212 | 202 | 204 | 307 |
| 3303 | 401 | 119 | 212 | 212 | 208 | 302 |
| 3304 | 402 | 74 | 210 | 201 | 201 | 303 |
| 3305 | 401 | 105 | 212 | 202 | 208 | 306 |
| 3306 | 403 | 85 | 212 | 212 | 201 | 307 |
| 3307 | 401 | 21 | 212 | 202 | 208 | 301 |
| 3308 | 401 | 63 | 210 | 202 | 211 | 308 |
| 3309 | 402 | 126 | 212 | 202 | 202 | 307 |
| 3310 | 406 | 36 | 212 | 201 | 201 | 301 |
| 3311 | 401 | 87 | 206 | 202 | 209 | 302 |
| 3312 | 403 | 59 | 206 | 201 | 201 | 306 |
| 3313 | 402 | 112 | 212 | 201 | 201 | 304 |
| 3314 | 401 | 48 | 212 | 202 | 207 | 303 |
| 3315 | 401 | 120 | 201 | 201 | 201 | 304 |
| 3316 | 404 | 14 | 203 | 201 | 201 | 308 |
| 3317 | 403 | 121 | 209 | 202 | 206 | 308 |
| 3318 | 406 | 39 | 204 | 212 | 208 | 302 |
| 3319 | 406 | 96 | 210 | 212 | 201 | 305 |
| 3320 | 403 | 53 | 204 | 201 | 201 | 303 |
| 3321 | 401 | 16 | 205 | 201 | 201 | 306 |
| 3322 | 401 | 102 | 201 | 201 | 201 | 308 |
| 3323 | 401 | 23 | 205 | 202 | 209 | 301 |
| 3324 | 401 | 85 | 204 | 212 | 209 | 301 |
| 3325 | 403 | 71 | 211 | 202 | 203 | 306 |
| 3326 | 407 | 54 | 212 | 201 | 201 | 302 |
| 3327 | 401 | 91 | 203 | 201 | 201 | 306 |
| 3328 | 405 | 32 | 211 | 212 | 201 | 301 |
| 3329 | 404 | 116 | 205 | 202 | 208 | 301 |
| 3330 | 401 | 77 | 204 | 212 | 204 | 301 |
| 3331 | 402 | 99 | 203 | 202 | 207 | 307 |
| 3332 | 407 | 88 | 206 | 201 | 201 | 301 |
| 3333 | 402 | 9 | 212 | 212 | 208 | 305 |
| 3334 | 402 | 64 | 212 | 201 | 201 | 308 |
| 3335 | 407 | 60 | 202 | 201 | 201 | 301 |
| 3336 | 401 | 37 | 204 | 202 | 209 | 306 |
| 3337 | 401 | 101 | 201 | 212 | 201 | 308 |
| 3338 | 402 | 86 | 205 | 201 | 201 | 303 |
| 3339 | 406 | 62 | 205 | 212 | 209 | 303 |
| 3340 | 407 | 109 | 204 | 201 | 201 | 303 |
| 3341 | 402 | 106 | 205 | 201 | 208 | 307 |
| 3342 | 406 | 84 | 203 | 201 | 207 | 303 |
| 3343 | 401 | 17 | 207 | 201 | 201 | 308 |
| 3344 | 405 | 86 | 212 | 212 | 203 | 301 |
| 3345 | 401 | 117 | 211 | 202 | 201 | 302 |
| 3346 | 401 | 123 | 201 | 201 | 201 | 307 |
| 3347 | 402 | 101 | 205 | 202 | 211 | 302 |
| 3348 | 401 | 125 | 201 | 212 | 202 | 308 |
| 3349 | 401 | 26 | 209 | 212 | 201 | 303 |
| 3350 | 401 | 96 | 211 | 201 | 201 | 301 |
| 3351 | 406 | 75 | 204 | 202 | 201 | 302 |
| 3352 | 401 | 50 | 212 | 202 | 201 | 307 |
| 3353 | 406 | 29 | 210 | 202 | 205 | 306 |
| 3354 | 404 | 111 | 204 | 202 | 207 | 308 |
| 3355 | 407 | 85 | 204 | 201 | 204 | 308 |
| 3356 | 401 | 98 | 209 | 201 | 201 | 307 |
| 3357 | 402 | 24 | 204 | 201 | 201 | 301 |
| 3358 | 406 | 6 | 210 | 202 | 202 | 306 |
| 3359 | 401 | 11 | 212 | 201 | 206 | 306 |
| 3360 | 406 | 130 | 206 | 202 | 208 | 306 |
| 3361 | 405 | 8 | 204 | 212 | 201 | 301 |
| 3362 | 405 | 18 | 202 | 202 | 207 | 306 |
| 3363 | 401 | 86 | 210 | 212 | 201 | 303 |
| 3364 | 401 | 108 | 205 | 201 | 204 | 302 |
| 3365 | 407 | 98 | 205 | 201 | 201 | 302 |
| 3366 | 401 | 82 | 209 | 202 | 201 | 302 |
| 3367 | 407 | 2 | 201 | 201 | 201 | 308 |
| 3368 | 401 | 20 | 201 | 201 | 201 | 305 |
| 3369 | 406 | 91 | 212 | 201 | 201 | 304 |
| 3370 | 401 | 129 | 211 | 202 | 201 | 302 |
| 3371 | 401 | 98 | 208 | 201 | 201 | 308 |
| 3372 | 407 | 7 | 204 | 201 | 201 | 307 |
| 3373 | 401 | 109 | 207 | 201 | 201 | 304 |
| 3374 | 407 | 109 | 207 | 201 | 201 | 306 |
| 3375 | 401 | 91 | 201 | 201 | 201 | 305 |
| 3376 | 404 | 114 | 212 | 202 | 206 | 304 |
| 3377 | 407 | 101 | 207 | 201 | 202 | 301 |
| 3378 | 406 | 66 | 201 | 201 | 208 | 304 |

Compounds having the formula:

TABLE 20

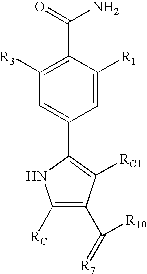

wherein R₁, R₃, R_{C}, R_{C1}, R₁₀, and R₇ are listed in Table 20.

| Compound | | | | | | |
|---|---|---|---|---|---|---|
| No. | R₁ | R₃ | R_{C} | R_{C1} | R₁₀ | R₇ |
| 3379 | 401 | 58 | 202 | 212 | 211 | 306 |
| 3380 | 406 | 96 | 201 | 201 | 201 | 308 |
| 3381 | 401 | 30 | 210 | 201 | 201 | 305 |
| 3382 | 405 | 98 | 204 | 202 | 208 | 301 |
| 3383 | 401 | 91 | 206 | 202 | 207 | 307 |
| 3384 | 401 | 80 | 204 | 212 | 206 | 306 |
| 3385 | 401 | 122 | 201 | 201 | 201 | 308 |
| 3386 | 404 | 42 | 205 | 212 | 207 | 308 |
| 3387 | 407 | 101 | 212 | 202 | 207 | 307 |
| 3388 | 403 | 101 | 211 | 201 | 201 | 306 |
| 3389 | 403 | 70 | 205 | 201 | 207 | 302 |
| 3390 | 401 | 109 | 201 | 212 | 208 | 301 |
| 3391 | 401 | 15 | 211 | 202 | 201 | 302 |
| 3392 | 405 | 88 | 210 | 201 | 201 | 301 |
| 3393 | 402 | 96 | 201 | 201 | 201 | 304 |
| 3394 | 401 | 98 | 212 | 201 | 201 | 307 |
| 3395 | 405 | 46 | 204 | 202 | 208 | 308 |
| 3396 | 401 | 90 | 205 | 202 | 201 | 305 |
| 3397 | 401 | 1 | 201 | 201 | 206 | 308 |
| 3398 | 403 | 25 | 204 | 202 | 201 | 306 |
| 3399 | 401 | 81 | 210 | 202 | 208 | 301 |
| 3400 | 405 | 104 | 210 | 202 | 204 | 307 |
| 3401 | 401 | 27 | 212 | 202 | 203 | 306 |
| 3402 | 404 | 12 | 207 | 201 | 201 | 303 |
| 3403 | 403 | 52 | 201 | 202 | 207 | 306 |
| 3404 | 401 | 65 | 212 | 212 | 202 | 306 |
| 3405 | 406 | 88 | 208 | 212 | 211 | 301 |
| 3406 | 402 | 109 | 212 | 201 | 201 | 305 |
| 3407 | 404 | 4 | 201 | 201 | 201 | 304 |
| 3408 | 401 | 57 | 202 | 212 | 208 | 308 |
| 3409 | 404 | 96 | 212 | 201 | 201 | 301 |
| 3410 | 402 | 97 | 210 | 201 | 201 | 304 |
| 3411 | 405 | 76 | 202 | 202 | 201 | 306 |
| 3412 | 407 | 3 | 203 | 202 | 201 | 302 |
| 3413 | 404 | 89 | 206 | 202 | 204 | 306 |
| 3414 | 404 | 28 | 212 | 202 | 209 | 302 |
| 3415 | 405 | 55 | 204 | 201 | 201 | 302 |
| 3416 | 403 | 92 | 204 | 202 | 209 | 301 |
| 3417 | 404 | 85 | 204 | 202 | 207 | 301 |
| 3418 | 401 | 85 | 210 | 202 | 211 | 303 |
| 3419 | 401 | 98 | 208 | 201 | 201 | 301 |
| 3420 | 405 | 88 | 204 | 202 | 208 | 306 |
| 3421 | 403 | 51 | 211 | 201 | 201 | 307 |
| 3422 | 405 | 47 | 203 | 202 | 203 | 306 |
| 3423 | 401 | 86 | 201 | 202 | 201 | 301 |
| 3424 | 402 | 33 | 203 | 202 | 209 | 302 |
| 3425 | 404 | 44 | 202 | 201 | 201 | 307 |
| 3426 | 401 | 40 | 210 | 202 | 203 | 306 |
| 3427 | 402 | 94 | 201 | 212 | 205 | 304 |
| 3428 | 401 | 41 | 202 | 202 | 201 | 302 |
| 3429 | 403 | 109 | 207 | 201 | 201 | 302 |
| 3430 | 401 | 127 | 210 | 201 | 201 | 301 |
| 3431 | 401 | 31 | 208 | 212 | 211 | 305 |
| 3432 | 401 | 45 | 205 | 201 | 201 | 303 |
| 3433 | 401 | 110 | 207 | 201 | 201 | 302 |
| 3434 | 405 | 103 | 205 | 201 | 201 | 301 |
| 3435 | 401 | 22 | 211 | 201 | 201 | 308 |
| 3436 | 403 | 96 | 209 | 201 | 201 | 302 |
| 3437 | 404 | 56 | 209 | 202 | 209 | 304 |
| 3438 | 402 | 79 | 206 | 201 | 207 | 301 |
| 3439 | 401 | 93 | 205 | 212 | 202 | 307 |
| 3440 | 405 | 35 | 209 | 202 | 201 | 301 |
| 3441 | 401 | 13 | 204 | 201 | 201 | 302 |
| 3442 | 406 | 128 | 212 | 201 | 201 | 303 |
| 3443 | 401 | 86 | 204 | 212 | 204 | 304 |
| 3444 | 401 | 115 | 206 | 201 | 201 | 308 |
| 3445 | 404 | 86 | 201 | 212 | 209 | 302 |
| 3446 | 405 | 85 | 211 | 202 | 201 | 301 |
| 3447 | 403 | 88 | 211 | 201 | 201 | 306 |
| 3448 | 401 | 101 | 203 | 201 | 201 | 301 |
| 3449 | 407 | 61 | 202 | 202 | 209 | 301 |
| 3450 | 402 | 5 | 205 | 201 | 201 | 302 |
| 3451 | 403 | 95 | 204 | 201 | 201 | 308 |
| 3452 | 404 | 100 | 212 | 202 | 202 | 308 |
| 3453 | 406 | 38 | 206 | 201 | 201 | 305 |
| 3454 | 407 | 88 | 209 | 201 | 209 | 307 |
| 3455 | 403 | 118 | 203 | 202 | 209 | 306 |
| 3456 | 404 | 113 | 210 | 201 | 201 | 307 |
| 3457 | 401 | 43 | 212 | 202 | 201 | 308 |
| 3458 | 401 | 73 | 210 | 202 | 209 | 308 |
| 3459 | 407 | 34 | 205 | 201 | 201 | 304 |
| 3460 | 401 | 69 | 210 | 201 | 201 | 303 |
| 3461 | 401 | 91 | 209 | 212 | 201 | 302 |
| 3462 | 404 | 91 | 210 | 201 | 201 | 308 |
| 3463 | 406 | 83 | 211 | 212 | 206 | 303 |
| 3464 | 407 | 109 | 204 | 212 | 207 | 303 |
| 3465 | 402 | 19 | 202 | 201 | 201 | 308 |
| 3466 | 401 | 67 | 211 | 201 | 201 | 302 |
| 3467 | 405 | 96 | 204 | 201 | 201 | 308 |
| 3468 | 407 | 107 | 204 | 201 | 201 | 302 |
| 3469 | 401 | 72 | 204 | 202 | 201 | 305 |
| 3470 | 405 | 10 | 211 | 202 | 201 | 308 |
| 3471 | 401 | 88 | 206 | 202 | 203 | 303 |
| 3472 | 401 | 78 | 204 | 201 | 201 | 307 |
| 3473 | 401 | 68 | 204 | 201 | 201 | 304 |
| 3474 | 401 | 124 | 203 | 201 | 201 | 308 |
| 3475 | 403 | 88 | 210 | 202 | 204 | 306 |
| 3476 | 406 | 49 | 209 | 201 | 201 | 307 |
| 3477 | 401 | 96 | 212 | 202 | 204 | 307 |
| 3478 | 401 | 119 | 212 | 212 | 208 | 302 |
| 3479 | 402 | 74 | 210 | 201 | 201 | 303 |
| 3480 | 401 | 105 | 212 | 202 | 208 | 306 |
| 3481 | 403 | 85 | 212 | 212 | 201 | 307 |
| 3482 | 401 | 21 | 212 | 202 | 208 | 301 |
| 3483 | 401 | 63 | 210 | 202 | 211 | 308 |
| 3484 | 402 | 126 | 212 | 202 | 202 | 307 |
| 3485 | 406 | 36 | 212 | 201 | 201 | 301 |
| 3486 | 401 | 87 | 206 | 202 | 209 | 302 |
| 3487 | 403 | 59 | 206 | 201 | 201 | 306 |
| 3488 | 402 | 112 | 212 | 201 | 201 | 304 |
| 3489 | 401 | 48 | 212 | 202 | 207 | 303 |
| 3490 | 401 | 120 | 201 | 201 | 201 | 304 |
| 3491 | 404 | 14 | 203 | 201 | 201 | 308 |
| 3492 | 403 | 121 | 209 | 202 | 206 | 308 |
| 3493 | 406 | 39 | 204 | 212 | 208 | 302 |
| 3494 | 406 | 96 | 210 | 212 | 201 | 305 |
| 3495 | 403 | 53 | 204 | 201 | 201 | 303 |
| 3496 | 401 | 16 | 205 | 201 | 201 | 306 |
| 3497 | 401 | 102 | 201 | 201 | 201 | 308 |
| 3498 | 401 | 23 | 205 | 202 | 209 | 301 |
| 3499 | 401 | 85 | 204 | 212 | 209 | 301 |
| 3500 | 403 | 71 | 211 | 202 | 203 | 306 |
| 3501 | 407 | 54 | 212 | 201 | 201 | 302 |
| 3502 | 401 | 91 | 203 | 201 | 201 | 306 |
| 3503 | 405 | 32 | 211 | 212 | 201 | 301 |
| 3504 | 404 | 116 | 205 | 202 | 208 | 301 |
| 3505 | 401 | 77 | 204 | 212 | 204 | 301 |
| 3506 | 402 | 99 | 203 | 202 | 207 | 307 |
| 3507 | 407 | 88 | 206 | 201 | 201 | 301 |
| 3508 | 402 | 9 | 212 | 212 | 208 | 305 |
| 3509 | 402 | 64 | 212 | 201 | 201 | 308 |
| 3510 | 407 | 60 | 202 | 201 | 201 | 301 |
| 3511 | 401 | 37 | 204 | 202 | 209 | 306 |
| 3512 | 401 | 101 | 201 | 212 | 201 | 308 |
| 3513 | 402 | 86 | 205 | 201 | 201 | 303 |
| 3514 | 406 | 62 | 205 | 212 | 209 | 303 |
| 3515 | 407 | 109 | 204 | 201 | 201 | 303 |
| 3516 | 402 | 106 | 205 | 201 | 208 | 307 |
| 3517 | 406 | 84 | 203 | 201 | 207 | 303 |
| 3518 | 401 | 17 | 207 | 201 | 201 | 308 |
| 3519 | 405 | 86 | 212 | 212 | 203 | 301 |
| 3520 | 401 | 117 | 211 | 202 | 201 | 302 |
| 3521 | 401 | 123 | 201 | 201 | 201 | 307 |
| 3522 | 402 | 101 | 205 | 202 | 211 | 302 |
| 3523 | 401 | 125 | 201 | 212 | 202 | 308 |
| 3524 | 401 | 26 | 209 | 212 | 201 | 303 |
| 3525 | 401 | 96 | 211 | 201 | 201 | 301 |
| 3526 | 406 | 75 | 204 | 202 | 201 | 302 |
| 3527 | 401 | 50 | 212 | 202 | 201 | 307 |
| 3528 | 406 | 29 | 210 | 202 | 205 | 306 |
| 3529 | 404 | 111 | 204 | 202 | 207 | 308 |
| 3530 | 407 | 85 | 204 | 201 | 204 | 308 |
| 3531 | 401 | 98 | 209 | 201 | 201 | 307 |
| 3532 | 402 | 24 | 204 | 201 | 201 | 301 |
| 3533 | 406 | 6 | 210 | 202 | 202 | 306 |
| 3534 | 401 | 11 | 212 | 201 | 206 | 306 |
| 3535 | 406 | 130 | 206 | 202 | 208 | 306 |
| 3536 | 405 | 8 | 204 | 212 | 201 | 301 |
| 3537 | 405 | 18 | 202 | 202 | 207 | 306 |
| 3538 | 401 | 86 | 210 | 212 | 201 | 303 |
| 3539 | 401 | 108 | 205 | 201 | 204 | 302 |
| 3540 | 407 | 98 | 205 | 201 | 201 | 302 |
| 3541 | 401 | 82 | 209 | 202 | 201 | 302 |
| 3542 | 407 | 2 | 201 | 201 | 201 | 308 |
| 3543 | 401 | 20 | 201 | 201 | 201 | 305 |
| 3544 | 406 | 91 | 212 | 201 | 201 | 304 |
| 3545 | 401 | 129 | 211 | 202 | 201 | 302 |
| 3546 | 401 | 98 | 208 | 201 | 201 | 308 |
| 3547 | 407 | 7 | 204 | 201 | 201 | 307 |
| 3548 | 401 | 109 | 207 | 201 | 201 | 304 |
| 3549 | 407 | 109 | 207 | 201 | 201 | 306 |
| 3550 | 401 | 91 | 201 | 201 | 201 | 305 |
| 3551 | 404 | 114 | 212 | 202 | 206 | 304 |
| 3552 | 407 | 101 | 207 | 201 | 202 | 301 |
| 3553 | 406 | 66 | 201 | 201 | 208 | 304 |

Compounds having the formula:

TABLE 21

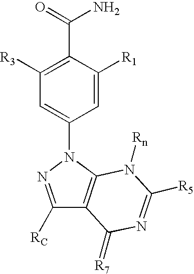

wherein R₁, R₃, R_{C}, Rₙ, R₅ and R₇ are listed in Table 21.

| Compound | | | | | | |
|---|---|---|---|---|---|---|
| No. | R₁ | R₃ | R_{C} | Rₙ | R₅ | R₇ |
| 3554 | 407 | 17 | 207 | 201 | 201 | 308 |
| 3555 | 404 | 33 | 203 | 202 | 202 | 302 |
| 3556 | 401 | 106 | 205 | 202 | 212 | 307 |
| 3557 | 404 | 88 | 211 | 201 | 201 | 306 |
| 3558 | 402 | 91 | 209 | 202 | 212 | 302 |
| 3559 | 402 | 75 | 204 | 202 | 202 | 302 |
| 3560 | 401 | 96 | 211 | 201 | 201 | 301 |
| 3561 | 401 | 92 | 204 | 201 | 212 | 301 |
| 3562 | 405 | 39 | 204 | 202 | 212 | 302 |
| 3563 | 404 | 80 | 204 | 202 | 212 | 306 |
| 3564 | 404 | 112 | 212 | 202 | 201 | 304 |
| 3565 | 406 | 97 | 210 | 202 | 201 | 304 |
| 3566 | 401 | 85 | 212 | 201 | 212 | 307 |
| 3567 | 401 | 98 | 208 | 212 | 201 | 301 |
| 3568 | 402 | 120 | 201 | 201 | 201 | 304 |
| 3569 | 401 | 108 | 205 | 201 | 212 | 302 |
| 3570 | 404 | 88 | 208 | 212 | 212 | 301 |
| 3571 | 402 | 96 | 201 | 202 | 201 | 304 |
| 3572 | 405 | 107 | 204 | 212 | 201 | 302 |
| 3573 | 402 | 128 | 212 | 201 | 201 | 303 |
| 3574 | 401 | 111 | 204 | 201 | 202 | 308 |
| 3575 | 405 | 34 | 205 | 202 | 201 | 304 |
| 3576 | 404 | 86 | 201 | 201 | 202 | 301 |
| 3577 | 401 | 85 | 204 | 212 | 202 | 301 |
| 3578 | 405 | 94 | 201 | 201 | 212 | 304 |
| 3579 | 406 | 86 | 212 | 212 | 212 | 301 |
| 3580 | 407 | 68 | 204 | 201 | 212 | 304 |
| 3581 | 401 | 58 | 202 | 202 | 212 | 306 |
| 3582 | 401 | 54 | 212 | 202 | 201 | 302 |
| 3583 | 403 | 109 | 201 | 202 | 212 | 301 |
| 3584 | 401 | 9 | 212 | 202 | 212 | 305 |
| 3585 | 401 | 66 | 201 | 201 | 212 | 304 |
| 3586 | 401 | 96 | 201 | 212 | 201 | 308 |
| 3587 | 401 | 86 | 201 | 202 | 212 | 302 |
| 3588 | 401 | 101 | 201 | 201 | 212 | 308 |
| 3589 | 401 | 84 | 203 | 202 | 212 | 303 |
| 3590 | 401 | 96 | 212 | 202 | 201 | 301 |
| 3591 | 401 | 109 | 207 | 202 | 201 | 302 |
| 3592 | 405 | 85 | 210 | 201 | 202 | 303 |
| 3593 | 401 | 20 | 201 | 201 | 201 | 305 |
| 3594 | 404 | 57 | 202 | 201 | 212 | 308 |
| 3595 | 402 | 60 | 202 | 201 | 201 | 301 |
| 3596 | 401 | 113 | 210 | 201 | 201 | 307 |
| 3597 | 405 | 109 | 207 | 202 | 201 | 304 |
| 3598 | 401 | 22 | 211 | 202 | 201 | 308 |
| 3599 | 403 | 89 | 206 | 201 | 202 | 306 |
| 3600 | 401 | 101 | 212 | 201 | 202 | 307 |
| 3601 | 401 | 109 | 207 | 201 | 201 | 306 |
| 3602 | 406 | 91 | 210 | 201 | 201 | 308 |
| 3603 | 401 | 91 | 212 | 201 | 201 | 304 |
| 3604 | 403 | 37 | 204 | 202 | 202 | 306 |
| 3605 | 405 | 114 | 212 | 202 | 202 | 304 |
| 3606 | 407 | 98 | 212 | 201 | 201 | 307 |
| 3607 | 403 | 46 | 204 | 202 | 202 | 308 |
| 3608 | 402 | 98 | 209 | 201 | 201 | 307 |
| 3609 | 407 | 73 | 210 | 202 | 202 | 308 |
| 3610 | 401 | 28 | 212 | 212 | 202 | 302 |
| 3611 | 403 | 129 | 211 | 202 | 202 | 302 |
| 3612 | 405 | 19 | 202 | 202 | 201 | 308 |
| 3613 | 401 | 79 | 206 | 201 | 212 | 301 |
| 3614 | 401 | 83 | 211 | 202 | 212 | 303 |
| 3615 | 402 | 103 | 205 | 202 | 201 | 301 |
| 3616 | 402 | 86 | 210 | 201 | 212 | 303 |
| 3617 | 404 | 51 | 211 | 201 | 201 | 307 |
| 3618 | 403 | 64 | 212 | 202 | 201 | 308 |
| 3619 | 407 | 65 | 212 | 201 | 212 | 306 |
| 3620 | 406 | 96 | 210 | 212 | 212 | 305 |
| 3621 | 403 | 8 | 204 | 201 | 212 | 301 |
| 3622 | 401 | 38 | 206 | 202 | 201 | 305 |
| 3623 | 401 | 72 | 204 | 201 | 202 | 305 |
| 3624 | 405 | 59 | 206 | 202 | 201 | 306 |
| 3625 | 406 | 29 | 210 | 212 | 202 | 306 |
| 3626 | 401 | 130 | 206 | 202 | 202 | 306 |
| 3627 | 405 | 104 | 210 | 201 | 202 | 307 |
| 3628 | 401 | 25 | 204 | 201 | 202 | 306 |
| 3629 | 407 | 98 | 204 | 201 | 212 | 301 |
| 3630 | 401 | 115 | 206 | 212 | 201 | 308 |
| 3631 | 405 | 44 | 202 | 212 | 201 | 307 |
| 3632 | 401 | 55 | 204 | 201 | 201 | 302 |
| 3633 | 406 | 81 | 210 | 212 | 212 | 301 |
| 3634 | 406 | 47 | 203 | 201 | 202 | 306 |
| 3635 | 401 | 88 | 204 | 201 | 202 | 306 |
| 3636 | 406 | 88 | 206 | 202 | 202 | 303 |
| 3637 | 406 | 96 | 204 | 201 | 201 | 308 |
| 3638 | 401 | 116 | 205 | 202 | 202 | 301 |
| 3639 | 407 | 90 | 205 | 201 | 212 | 305 |
| 3640 | 401 | 24 | 204 | 212 | 201 | 301 |
| 3641 | 401 | 76 | 202 | 212 | 202 | 306 |
| 3642 | 404 | 121 | 209 | 202 | 202 | 308 |
| 3643 | 401 | 93 | 205 | 201 | 212 | 307 |
| 3644 | 401 | 101 | 203 | 201 | 201 | 301 |
| 3645 | 402 | 91 | 203 | 212 | 201 | 306 |
| 3646 | 401 | 101 | 205 | 212 | 202 | 302 |
| 3647 | 404 | 32 | 211 | 202 | 212 | 301 |
| 3648 | 403 | 6 | 210 | 202 | 202 | 306 |
| 3649 | 404 | 109 | 204 | 202 | 201 | 303 |
| 3650 | 401 | 12 | 207 | 202 | 201 | 303 |
| 3651 | 405 | 101 | 207 | 202 | 212 | 301 |
| 3652 | 407 | 86 | 205 | 201 | 201 | 303 |
| 3653 | 402 | 18 | 202 | 201 | 202 | 306 |
| 3654 | 406 | 96 | 212 | 201 | 212 | 307 |
| 3655 | 401 | 82 | 209 | 212 | 202 | 302 |
| 3656 | 404 | 85 | 204 | 201 | 212 | 308 |
| 3657 | 401 | 127 | 210 | 201 | 201 | 301 |
| 3658 | 405 | 88 | 206 | 202 | 212 | 301 |
| 3659 | 402 | 63 | 210 | 201 | 202 | 308 |
| 3660 | 403 | 16 | 205 | 201 | 201 | 306 |
| 3661 | 401 | 11 | 212 | 202 | 212 | 306 |
| 3662 | 407 | 99 | 203 | 212 | 202 | 307 |
| 3663 | 402 | 31 | 208 | 201 | 212 | 305 |
| 3664 | 405 | 98 | 205 | 202 | 201 | 302 |
| 3665 | 405 | 3 | 203 | 201 | 202 | 302 |
| 3666 | 406 | 10 | 211 | 201 | 202 | 308 |
| 3667 | 403 | 87 | 206 | 212 | 202 | 302 |
| 3668 | 401 | 35 | 209 | 201 | 202 | 301 |
| 3669 | 405 | 26 | 209 | 202 | 212 | 303 |
| 3670 | 407 | 74 | 210 | 201 | 201 | 303 |
| 3671 | 407 | 36 | 212 | 202 | 201 | 301 |
| 3672 | 405 | 56 | 209 | 212 | 202 | 304 |
| 3673 | 403 | 88 | 209 | 201 | 212 | 307 |
| 3674 | 402 | 95 | 204 | 201 | 201 | 308 |
| 3675 | 406 | 96 | 209 | 202 | 201 | 302 |
| 3676 | 404 | 61 | 202 | 201 | 202 | 301 |
| 3677 | 401 | 77 | 204 | 201 | 212 | 301 |
| 3678 | 401 | 27 | 212 | 202 | 202 | 306 |
| 3679 | 403 | 105 | 212 | 201 | 202 | 306 |
| 3680 | 401 | 69 | 210 | 202 | 201 | 303 |
| 3681 | 401 | 41 | 202 | 202 | 202 | 302 |
| 3682 | 404 | 4 | 201 | 202 | 201 | 304 |
| 3683 | 401 | 7 | 204 | 202 | 201 | 307 |
| 3684 | 401 | 86 | 204 | 202 | 212 | 304 |
| 3685 | 401 | 23 | 205 | 202 | 202 | 301 |
| 3686 | 401 | 118 | 203 | 201 | 212 | 306 |
| 3687 | 401 | 101 | 211 | 201 | 201 | 306 |
| 3688 | 406 | 62 | 205 | 212 | 212 | 303 |
| 3689 | 401 | 78 | 204 | 201 | 201 | 307 |
| 3690 | 404 | 1 | 201 | 202 | 212 | 308 |
| 3691 | 401 | 70 | 205 | 212 | 201 | 302 |
| 3692 | 401 | 50 | 212 | 212 | 202 | 307 |
| 3693 | 407 | 91 | 206 | 201 | 212 | 307 |
| 3694 | 401 | 125 | 201 | 201 | 212 | 308 |
| 3695 | 403 | 102 | 201 | 201 | 201 | 308 |
| 3696 | 402 | 45 | 205 | 201 | 201 | 303 |
| 3697 | 401 | 88 | 210 | 212 | 202 | 306 |
| 3698 | 401 | 88 | 210 | 202 | 201 | 301 |
| 3699 | 401 | 85 | 211 | 212 | 212 | 301 |
| 3700 | 404 | 15 | 211 | 201 | 212 | 302 |
| 3701 | 407 | 67 | 211 | 201 | 201 | 302 |
| 3702 | 403 | 40 | 210 | 201 | 202 | 306 |
| 3703 | 407 | 30 | 210 | 201 | 201 | 305 |
| 3704 | 401 | 53 | 204 | 201 | 201 | 303 |
| 3705 | 401 | 48 | 212 | 201 | 202 | 303 |
| 3706 | 406 | 122 | 201 | 201 | 201 | 308 |
| 3707 | 401 | 123 | 201 | 201 | 201 | 307 |
| 3708 | 407 | 43 | 212 | 201 | 202 | 308 |
| 3709 | 404 | 109 | 212 | 212 | 201 | 305 |
| 3710 | 402 | 14 | 203 | 212 | 201 | 308 |
| 3711 | 401 | 13 | 204 | 212 | 201 | 302 |
| 3712 | 401 | 117 | 211 | 202 | 202 | 302 |
| 3713 | 406 | 5 | 205 | 201 | 201 | 302 |
| 3714 | 402 | 124 | 203 | 201 | 201 | 308 |
| 3715 | 401 | 98 | 208 | 202 | 201 | 308 |
| 3716 | 406 | 109 | 204 | 202 | 212 | 303 |
| 3717 | 401 | 52 | 201 | 201 | 202 | 306 |
| 3718 | 406 | 42 | 205 | 201 | 212 | 308 |
| 3719 | 407 | 126 | 212 | 202 | 202 | 307 |
| 3720 | 403 | 119 | 212 | 201 | 212 | 302 |
| 3721 | 403 | 49 | 209 | 202 | 201 | 307 |
| 3722 | 401 | 100 | 212 | 212 | 212 | 308 |
| 3723 | 402 | 21 | 212 | 201 | 202 | 301 |
| 3724 | 401 | 91 | 201 | 202 | 201 | 305 |
| 3725 | 403 | 2 | 201 | 201 | 201 | 308 |
| 3726 | 401 | 71 | 211 | 212 | 202 | 306 |
| 3727 | 401 | 110 | 207 | 201 | 201 | 302 |
| 3728 | 407 | 85 | 204 | 201 | 212 | 301 |

Compounds having the formula:

TABLE 22

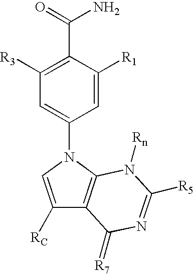

wherein R₁, R₃, R_{C}, Rₙ, R₅ and R₇ are listed in Table 22.

| Compound | | | | | | |
|---|---|---|---|---|---|---|
| No. | R₁ | R₃ | R_{C} | Rₙ | R₅ | R₇ |
| 3729 | 407 | 17 | 207 | 201 | 201 | 308 |
| 3730 | 404 | 33 | 203 | 202 | 202 | 302 |
| 3731 | 401 | 106 | 205 | 202 | 212 | 307 |
| 3732 | 404 | 88 | 211 | 201 | 201 | 306 |
| 3733 | 402 | 91 | 209 | 202 | 212 | 302 |
| 3734 | 402 | 75 | 204 | 202 | 202 | 302 |
| 3735 | 401 | 96 | 211 | 201 | 201 | 301 |
| 3736 | 401 | 92 | 204 | 201 | 212 | 301 |
| 3737 | 405 | 39 | 204 | 202 | 212 | 302 |
| 3738 | 404 | 80 | 204 | 202 | 212 | 306 |
| 3739 | 404 | 112 | 212 | 202 | 201 | 304 |
| 3740 | 406 | 97 | 210 | 202 | 201 | 304 |
| 3741 | 401 | 85 | 212 | 201 | 212 | 307 |
| 3742 | 401 | 98 | 208 | 212 | 201 | 301 |
| 3743 | 402 | 120 | 201 | 201 | 201 | 304 |
| 3744 | 401 | 108 | 205 | 201 | 212 | 302 |
| 3745 | 404 | 88 | 208 | 212 | 212 | 301 |
| 3746 | 402 | 96 | 201 | 202 | 201 | 304 |
| 3747 | 405 | 107 | 204 | 212 | 201 | 302 |
| 3748 | 402 | 128 | 212 | 201 | 201 | 303 |
| 3749 | 401 | 111 | 204 | 201 | 202 | 308 |
| 3750 | 405 | 34 | 205 | 202 | 201 | 304 |
| 3751 | 404 | 86 | 201 | 201 | 202 | 301 |
| 3752 | 401 | 85 | 204 | 212 | 202 | 301 |
| 3753 | 405 | 94 | 201 | 201 | 212 | 304 |
| 3754 | 406 | 86 | 212 | 212 | 212 | 301 |
| 3755 | 407 | 68 | 204 | 201 | 212 | 304 |
| 3756 | 401 | 58 | 202 | 202 | 212 | 306 |
| 3757 | 401 | 54 | 212 | 202 | 201 | 302 |
| 3758 | 403 | 109 | 201 | 202 | 212 | 301 |
| 3759 | 401 | 9 | 212 | 202 | 212 | 305 |
| 3760 | 401 | 66 | 201 | 201 | 212 | 304 |
| 3761 | 401 | 96 | 201 | 212 | 201 | 308 |
| 3762 | 401 | 86 | 201 | 202 | 212 | 302 |
| 3763 | 401 | 101 | 201 | 201 | 212 | 308 |
| 3764 | 401 | 84 | 203 | 202 | 212 | 303 |
| 3765 | 401 | 96 | 212 | 202 | 201 | 301 |
| 3766 | 401 | 109 | 207 | 202 | 201 | 302 |
| 3767 | 405 | 85 | 210 | 201 | 202 | 303 |
| 3768 | 401 | 20 | 201 | 201 | 201 | 305 |
| 3769 | 404 | 57 | 202 | 201 | 212 | 308 |
| 3770 | 402 | 60 | 202 | 201 | 201 | 301 |
| 3771 | 401 | 113 | 210 | 201 | 201 | 307 |
| 3772 | 405 | 109 | 207 | 202 | 201 | 304 |
| 3773 | 401 | 22 | 211 | 202 | 201 | 308 |
| 3774 | 403 | 89 | 206 | 201 | 202 | 306 |
| 3775 | 401 | 101 | 212 | 201 | 202 | 307 |
| 3776 | 401 | 109 | 207 | 201 | 201 | 306 |
| 3777 | 406 | 91 | 210 | 201 | 201 | 308 |
| 3778 | 401 | 91 | 212 | 201 | 201 | 304 |
| 3779 | 403 | 37 | 204 | 202 | 202 | 306 |
| 3780 | 405 | 114 | 212 | 202 | 202 | 304 |
| 3781 | 407 | 98 | 212 | 201 | 201 | 307 |
| 3782 | 403 | 46 | 204 | 202 | 202 | 308 |
| 3783 | 402 | 98 | 209 | 201 | 201 | 307 |
| 3784 | 407 | 73 | 210 | 202 | 202 | 308 |
| 3785 | 401 | 28 | 212 | 212 | 202 | 302 |
| 3786 | 403 | 129 | 211 | 202 | 202 | 302 |
| 3787 | 405 | 19 | 202 | 202 | 201 | 308 |
| 3788 | 401 | 79 | 206 | 201 | 212 | 301 |
| 3789 | 401 | 83 | 211 | 202 | 212 | 303 |
| 3790 | 402 | 103 | 205 | 202 | 201 | 301 |
| 3791 | 402 | 86 | 210 | 201 | 212 | 303 |
| 3792 | 404 | 51 | 211 | 201 | 201 | 307 |
| 3793 | 403 | 64 | 212 | 202 | 201 | 308 |
| 3794 | 407 | 65 | 212 | 201 | 212 | 306 |
| 3795 | 406 | 96 | 210 | 212 | 212 | 305 |
| 3796 | 403 | 8 | 204 | 201 | 212 | 301 |
| 3797 | 401 | 38 | 206 | 202 | 201 | 305 |
| 3798 | 401 | 72 | 204 | 201 | 202 | 305 |
| 3799 | 405 | 59 | 206 | 202 | 201 | 306 |
| 3800 | 406 | 29 | 210 | 212 | 202 | 306 |
| 3801 | 401 | 130 | 206 | 202 | 202 | 306 |
| 3802 | 405 | 104 | 210 | 201 | 202 | 307 |
| 3803 | 401 | 25 | 204 | 201 | 202 | 306 |
| 3804 | 407 | 98 | 204 | 201 | 212 | 301 |
| 3805 | 401 | 115 | 206 | 212 | 201 | 308 |
| 3806 | 405 | 44 | 202 | 212 | 201 | 307 |
| 3807 | 401 | 55 | 204 | 201 | 201 | 302 |
| 3808 | 406 | 81 | 210 | 212 | 212 | 301 |
| 3809 | 406 | 47 | 203 | 201 | 202 | 306 |
| 3810 | 401 | 88 | 204 | 201 | 202 | 306 |
| 3811 | 406 | 88 | 206 | 202 | 202 | 303 |
| 3812 | 406 | 96 | 204 | 201 | 201 | 308 |
| 3813 | 401 | 116 | 205 | 202 | 202 | 301 |
| 3814 | 407 | 90 | 205 | 201 | 212 | 305 |
| 3815 | 401 | 24 | 204 | 212 | 201 | 301 |
| 3816 | 401 | 76 | 202 | 212 | 202 | 306 |
| 3817 | 404 | 121 | 209 | 202 | 202 | 308 |
| 3818 | 401 | 93 | 205 | 201 | 212 | 307 |
| 3819 | 401 | 101 | 203 | 201 | 201 | 301 |
| 3820 | 402 | 91 | 203 | 212 | 201 | 306 |
| 3821 | 401 | 101 | 205 | 212 | 202 | 302 |
| 3822 | 404 | 32 | 211 | 202 | 212 | 301 |
| 3823 | 403 | 6 | 210 | 202 | 202 | 306 |
| 3824 | 404 | 109 | 204 | 202 | 201 | 303 |
| 3825 | 401 | 12 | 207 | 202 | 201 | 303 |
| 3826 | 405 | 101 | 207 | 202 | 212 | 301 |
| 3827 | 407 | 86 | 205 | 201 | 201 | 303 |
| 3828 | 402 | 18 | 202 | 201 | 202 | 306 |
| 3829 | 406 | 96 | 212 | 201 | 212 | 307 |
| 3830 | 401 | 82 | 209 | 212 | 202 | 302 |
| 3831 | 404 | 85 | 204 | 201 | 212 | 308 |
| 3832 | 401 | 127 | 210 | 201 | 201 | 301 |
| 3833 | 405 | 88 | 206 | 202 | 212 | 301 |
| 3834 | 402 | 63 | 210 | 201 | 202 | 308 |
| 3835 | 403 | 16 | 205 | 201 | 201 | 306 |
| 3836 | 401 | 11 | 212 | 202 | 212 | 306 |
| 3837 | 407 | 99 | 203 | 212 | 202 | 307 |
| 3838 | 402 | 31 | 208 | 201 | 212 | 305 |
| 3839 | 405 | 98 | 205 | 202 | 201 | 302 |
| 3840 | 405 | 3 | 203 | 201 | 202 | 302 |
| 3841 | 406 | 10 | 211 | 201 | 202 | 308 |
| 3842 | 403 | 87 | 206 | 212 | 202 | 302 |
| 3843 | 401 | 35 | 209 | 201 | 202 | 301 |
| 3844 | 405 | 26 | 209 | 202 | 212 | 303 |
| 3845 | 407 | 74 | 210 | 201 | 201 | 303 |
| 3846 | 407 | 36 | 212 | 202 | 201 | 301 |
| 3847 | 405 | 56 | 209 | 212 | 202 | 304 |
| 3848 | 403 | 88 | 209 | 201 | 212 | 307 |
| 3849 | 402 | 95 | 204 | 201 | 201 | 308 |
| 3850 | 406 | 96 | 209 | 202 | 201 | 302 |
| 3851 | 404 | 61 | 202 | 201 | 202 | 301 |
| 3852 | 401 | 77 | 204 | 201 | 212 | 301 |
| 3853 | 401 | 27 | 212 | 202 | 202 | 306 |
| 3854 | 403 | 105 | 212 | 201 | 202 | 306 |
| 3855 | 401 | 69 | 210 | 202 | 201 | 303 |
| 3856 | 401 | 41 | 202 | 202 | 202 | 302 |
| 3857 | 404 | 4 | 201 | 202 | 201 | 304 |
| 3858 | 401 | 7 | 204 | 202 | 201 | 307 |
| 3859 | 401 | 86 | 204 | 202 | 212 | 304 |
| 3860 | 401 | 23 | 205 | 202 | 202 | 301 |
| 3861 | 401 | 118 | 203 | 201 | 212 | 306 |
| 3862 | 401 | 101 | 211 | 201 | 201 | 306 |
| 3863 | 406 | 62 | 205 | 212 | 212 | 303 |
| 3864 | 401 | 78 | 204 | 201 | 201 | 307 |
| 3865 | 404 | 1 | 201 | 202 | 212 | 308 |
| 3866 | 401 | 70 | 205 | 212 | 201 | 302 |
| 3867 | 401 | 50 | 212 | 212 | 202 | 307 |
| 3868 | 407 | 91 | 206 | 201 | 212 | 307 |
| 3869 | 401 | 125 | 201 | 201 | 212 | 308 |
| 3870 | 403 | 102 | 201 | 201 | 201 | 308 |
| 3871 | 402 | 45 | 205 | 201 | 201 | 303 |
| 3872 | 401 | 88 | 210 | 212 | 202 | 306 |
| 3873 | 401 | 88 | 210 | 202 | 201 | 301 |
| 3874 | 401 | 85 | 211 | 212 | 212 | 301 |
| 3875 | 404 | 15 | 211 | 201 | 212 | 302 |
| 3876 | 407 | 67 | 211 | 201 | 201 | 302 |
| 3877 | 403 | 40 | 210 | 201 | 202 | 306 |
| 3878 | 407 | 30 | 210 | 201 | 201 | 305 |
| 3879 | 401 | 53 | 204 | 201 | 201 | 303 |
| 3880 | 401 | 48 | 212 | 201 | 202 | 303 |
| 3881 | 406 | 122 | 201 | 201 | 201 | 308 |
| 3882 | 401 | 123 | 201 | 201 | 201 | 307 |
| 3883 | 407 | 43 | 212 | 201 | 202 | 308 |
| 3884 | 404 | 109 | 212 | 212 | 201 | 305 |
| 3885 | 402 | 14 | 203 | 212 | 201 | 308 |
| 3886 | 401 | 13 | 204 | 212 | 201 | 302 |
| 3887 | 401 | 117 | 211 | 202 | 202 | 302 |
| 3888 | 406 | 5 | 205 | 201 | 201 | 302 |
| 3889 | 402 | 124 | 203 | 201 | 201 | 308 |
| 3890 | 401 | 98 | 208 | 202 | 201 | 308 |
| 3891 | 406 | 109 | 204 | 202 | 212 | 303 |
| 3892 | 401 | 52 | 201 | 201 | 202 | 306 |
| 3893 | 406 | 42 | 205 | 201 | 212 | 308 |
| 3894 | 407 | 126 | 212 | 202 | 202 | 307 |
| 3895 | 403 | 119 | 212 | 201 | 212 | 302 |
| 3896 | 403 | 49 | 209 | 202 | 201 | 307 |
| 3897 | 401 | 100 | 212 | 212 | 212 | 308 |
| 3898 | 402 | 21 | 212 | 201 | 202 | 301 |
| 3899 | 401 | 91 | 201 | 202 | 201 | 305 |
| 3900 | 403 | 2 | 201 | 201 | 201 | 308 |
| 3901 | 401 | 71 | 211 | 212 | 202 | 306 |
| 3902 | 401 | 110 | 207 | 201 | 201 | 302 |
| 3903 | 407 | 85 | 204 | 201 | 212 | 301 |

Compounds having the formula:

TABLE 23

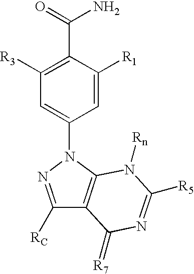

wherein R₁, R₃, R_{C}, Rₙ, R₅ and R₇ are listed in Table 23.

| Compound | | | | | | |
|---|---|---|---|---|---|---|
| No. | R₁ | R₃ | R_{C} | Rₙ | R₅ | R₇ |
| 3904 | 407 | 17 | 207 | 201 | 201 | 308 |
| 3905 | 404 | 33 | 203 | 202 | 202 | 302 |
| 3906 | 401 | 106 | 205 | 202 | 212 | 307 |
| 3907 | 404 | 88 | 211 | 201 | 201 | 306 |
| 3908 | 402 | 91 | 209 | 202 | 212 | 302 |
| 3909 | 402 | 75 | 204 | 202 | 202 | 302 |
| 3910 | 401 | 96 | 211 | 201 | 201 | 301 |
| 3911 | 401 | 92 | 204 | 201 | 212 | 301 |
| 3912 | 405 | 39 | 204 | 202 | 212 | 302 |
| 3913 | 404 | 80 | 204 | 202 | 212 | 306 |
| 3914 | 404 | 112 | 212 | 202 | 201 | 304 |
| 3915 | 406 | 97 | 210 | 202 | 201 | 304 |
| 3916 | 401 | 85 | 212 | 201 | 212 | 307 |
| 3917 | 401 | 98 | 208 | 212 | 201 | 301 |
| 3918 | 402 | 120 | 201 | 201 | 201 | 304 |
| 3919 | 401 | 108 | 205 | 201 | 212 | 302 |
| 3920 | 404 | 88 | 208 | 212 | 212 | 301 |
| 3921 | 402 | 96 | 201 | 202 | 201 | 304 |
| 3922 | 405 | 107 | 204 | 212 | 201 | 302 |
| 3923 | 402 | 128 | 212 | 201 | 201 | 303 |
| 3924 | 401 | 111 | 204 | 201 | 202 | 308 |
| 3925 | 405 | 34 | 205 | 202 | 201 | 304 |
| 3926 | 404 | 86 | 201 | 201 | 202 | 301 |
| 3927 | 401 | 85 | 204 | 212 | 202 | 301 |
| 3928 | 405 | 94 | 201 | 201 | 212 | 304 |
| 3929 | 406 | 86 | 212 | 212 | 212 | 301 |
| 3930 | 407 | 68 | 204 | 201 | 212 | 304 |
| 3931 | 401 | 58 | 202 | 202 | 212 | 306 |
| 3932 | 401 | 54 | 212 | 202 | 201 | 302 |
| 3933 | 403 | 109 | 201 | 202 | 212 | 301 |
| 3934 | 401 | 9 | 212 | 202 | 212 | 305 |
| 3935 | 401 | 66 | 201 | 201 | 212 | 304 |
| 3936 | 401 | 96 | 201 | 212 | 201 | 308 |
| 3937 | 401 | 86 | 201 | 202 | 212 | 302 |
| 3938 | 401 | 101 | 201 | 201 | 212 | 308 |
| 3939 | 401 | 84 | 203 | 202 | 212 | 303 |
| 3940 | 401 | 96 | 212 | 202 | 201 | 301 |
| 3941 | 401 | 109 | 207 | 202 | 201 | 302 |
| 3942 | 405 | 85 | 210 | 201 | 202 | 303 |
| 3943 | 401 | 20 | 201 | 201 | 201 | 305 |
| 3944 | 404 | 57 | 202 | 201 | 212 | 308 |
| 3945 | 402 | 60 | 202 | 201 | 201 | 301 |
| 3946 | 401 | 113 | 210 | 201 | 201 | 307 |
| 3947 | 405 | 109 | 207 | 202 | 201 | 304 |
| 3948 | 401 | 22 | 211 | 202 | 201 | 308 |
| 3949 | 403 | 89 | 206 | 201 | 202 | 306 |
| 3950 | 401 | 101 | 212 | 201 | 202 | 307 |
| 3951 | 401 | 109 | 207 | 201 | 201 | 306 |
| 3952 | 406 | 91 | 210 | 201 | 201 | 308 |
| 3953 | 401 | 91 | 212 | 201 | 201 | 304 |
| 3954 | 403 | 37 | 204 | 202 | 202 | 306 |
| 3955 | 405 | 114 | 212 | 202 | 202 | 304 |
| 3956 | 407 | 98 | 212 | 201 | 201 | 307 |
| 3957 | 403 | 46 | 204 | 202 | 202 | 308 |
| 3958 | 402 | 98 | 209 | 201 | 201 | 307 |
| 3959 | 407 | 73 | 210 | 202 | 202 | 308 |
| 3960 | 401 | 28 | 212 | 212 | 202 | 302 |
| 3961 | 403 | 129 | 211 | 202 | 202 | 302 |
| 3962 | 405 | 19 | 202 | 202 | 201 | 308 |
| 3963 | 401 | 79 | 206 | 201 | 212 | 301 |
| 3964 | 401 | 83 | 211 | 202 | 212 | 303 |
| 3965 | 402 | 103 | 205 | 202 | 201 | 301 |
| 3966 | 402 | 86 | 210 | 201 | 212 | 303 |
| 3967 | 404 | 51 | 211 | 201 | 201 | 307 |
| 3968 | 403 | 64 | 212 | 202 | 201 | 308 |
| 3969 | 407 | 65 | 212 | 201 | 212 | 306 |
| 3970 | 406 | 96 | 210 | 212 | 212 | 305 |
| 3971 | 403 | 8 | 204 | 201 | 212 | 301 |
| 3972 | 401 | 38 | 206 | 202 | 201 | 305 |
| 3973 | 401 | 72 | 204 | 201 | 202 | 305 |
| 3974 | 405 | 59 | 206 | 202 | 201 | 306 |
| 3975 | 406 | 29 | 210 | 212 | 202 | 306 |
| 3976 | 401 | 130 | 206 | 202 | 202 | 306 |
| 3977 | 405 | 104 | 210 | 201 | 202 | 307 |
| 3978 | 401 | 25 | 204 | 201 | 202 | 306 |
| 3979 | 407 | 98 | 204 | 201 | 212 | 301 |
| 3980 | 401 | 115 | 206 | 212 | 201 | 308 |
| 3981 | 405 | 44 | 202 | 212 | 201 | 307 |
| 3982 | 401 | 55 | 204 | 201 | 201 | 302 |
| 3983 | 406 | 81 | 210 | 212 | 212 | 301 |
| 3984 | 406 | 47 | 203 | 201 | 202 | 306 |
| 3985 | 401 | 88 | 204 | 201 | 202 | 306 |
| 3986 | 406 | 88 | 206 | 202 | 202 | 303 |
| 3987 | 406 | 96 | 204 | 201 | 201 | 308 |
| 3988 | 401 | 116 | 205 | 202 | 202 | 301 |
| 3989 | 407 | 90 | 205 | 201 | 212 | 305 |
| 3990 | 401 | 24 | 204 | 212 | 201 | 301 |
| 3991 | 401 | 76 | 202 | 212 | 202 | 306 |
| 3992 | 404 | 121 | 209 | 202 | 202 | 308 |
| 3993 | 401 | 93 | 205 | 201 | 212 | 307 |
| 3994 | 401 | 101 | 203 | 201 | 201 | 301 |
| 3995 | 402 | 91 | 203 | 212 | 201 | 306 |
| 3996 | 401 | 101 | 205 | 212 | 202 | 302 |
| 3997 | 404 | 32 | 211 | 202 | 212 | 301 |
| 3998 | 403 | 6 | 210 | 202 | 202 | 306 |
| 3999 | 404 | 109 | 204 | 202 | 201 | 303 |
| 4000 | 401 | 12 | 207 | 202 | 201 | 303 |
| 4001 | 405 | 101 | 207 | 202 | 212 | 301 |
| 4002 | 407 | 86 | 205 | 201 | 201 | 303 |
| 4003 | 402 | 18 | 202 | 201 | 202 | 306 |
| 4004 | 406 | 96 | 212 | 201 | 212 | 307 |
| 4005 | 401 | 82 | 209 | 212 | 202 | 302 |
| 4006 | 404 | 85 | 204 | 201 | 212 | 308 |
| 4007 | 401 | 127 | 210 | 201 | 201 | 301 |
| 4008 | 405 | 88 | 206 | 202 | 212 | 301 |
| 4009 | 402 | 63 | 210 | 201 | 202 | 308 |
| 4010 | 403 | 16 | 205 | 201 | 201 | 306 |
| 4011 | 401 | 11 | 212 | 202 | 212 | 306 |
| 4012 | 407 | 99 | 203 | 212 | 202 | 307 |
| 4013 | 402 | 31 | 208 | 201 | 212 | 305 |
| 4014 | 405 | 98 | 205 | 202 | 201 | 302 |
| 4015 | 405 | 3 | 203 | 201 | 202 | 302 |
| 4016 | 406 | 10 | 211 | 201 | 202 | 308 |
| 4017 | 403 | 87 | 206 | 212 | 202 | 302 |
| 4018 | 401 | 35 | 209 | 201 | 202 | 301 |
| 4019 | 405 | 26 | 209 | 202 | 212 | 303 |
| 4020 | 407 | 74 | 210 | 201 | 201 | 303 |
| 4021 | 407 | 36 | 212 | 202 | 201 | 301 |
| 4022 | 405 | 56 | 209 | 212 | 202 | 304 |
| 4023 | 403 | 88 | 209 | 201 | 212 | 307 |
| 4024 | 402 | 95 | 204 | 201 | 201 | 308 |
| 4025 | 406 | 96 | 209 | 202 | 201 | 302 |
| 4026 | 404 | 61 | 202 | 201 | 202 | 301 |
| 4027 | 401 | 77 | 204 | 201 | 212 | 301 |
| 4028 | 401 | 27 | 212 | 202 | 202 | 306 |
| 4029 | 403 | 105 | 212 | 201 | 202 | 306 |
| 4030 | 401 | 69 | 210 | 202 | 201 | 303 |
| 4031 | 401 | 41 | 202 | 202 | 202 | 302 |
| 4032 | 404 | 4 | 201 | 202 | 201 | 304 |
| 4033 | 401 | 7 | 204 | 202 | 201 | 307 |
| 4034 | 401 | 86 | 204 | 202 | 212 | 304 |
| 4035 | 401 | 23 | 205 | 202 | 202 | 301 |
| 4036 | 401 | 118 | 203 | 201 | 212 | 306 |
| 4037 | 401 | 101 | 211 | 201 | 201 | 306 |
| 4038 | 406 | 62 | 205 | 212 | 212 | 303 |
| 4039 | 401 | 78 | 204 | 201 | 201 | 307 |
| 4040 | 404 | 1 | 201 | 202 | 212 | 308 |
| 4041 | 401 | 70 | 205 | 212 | 201 | 302 |
| 4042 | 401 | 50 | 212 | 212 | 202 | 307 |
| 4043 | 407 | 91 | 206 | 201 | 212 | 307 |
| 4044 | 401 | 125 | 201 | 201 | 212 | 308 |
| 4045 | 403 | 102 | 201 | 201 | 201 | 308 |
| 4046 | 402 | 45 | 205 | 201 | 201 | 303 |
| 4047 | 401 | 88 | 210 | 212 | 202 | 306 |
| 4048 | 401 | 88 | 210 | 202 | 201 | 301 |
| 4049 | 401 | 85 | 211 | 212 | 212 | 301 |
| 4050 | 404 | 15 | 211 | 201 | 212 | 302 |
| 4051 | 407 | 67 | 211 | 201 | 201 | 302 |
| 4052 | 403 | 40 | 210 | 201 | 202 | 306 |
| 4053 | 407 | 30 | 210 | 201 | 201 | 305 |
| 4054 | 401 | 53 | 204 | 201 | 201 | 303 |
| 4055 | 401 | 48 | 212 | 201 | 202 | 303 |
| 4056 | 406 | 122 | 201 | 201 | 201 | 308 |
| 4057 | 401 | 123 | 201 | 201 | 201 | 307 |
| 4058 | 407 | 43 | 212 | 201 | 202 | 308 |
| 4059 | 404 | 109 | 212 | 212 | 201 | 305 |
| 4060 | 402 | 14 | 203 | 212 | 201 | 308 |
| 4061 | 401 | 13 | 204 | 212 | 201 | 302 |
| 4062 | 401 | 117 | 211 | 202 | 202 | 302 |
| 4063 | 406 | 5 | 205 | 201 | 201 | 302 |
| 4064 | 402 | 124 | 203 | 201 | 201 | 308 |
| 4065 | 401 | 98 | 208 | 202 | 201 | 308 |
| 4066 | 406 | 109 | 204 | 202 | 212 | 303 |
| 4067 | 401 | 52 | 201 | 201 | 202 | 306 |
| 4068 | 406 | 42 | 205 | 201 | 212 | 308 |
| 4069 | 407 | 126 | 212 | 202 | 202 | 307 |
| 4070 | 403 | 119 | 212 | 201 | 212 | 302 |
| 4071 | 403 | 49 | 209 | 202 | 201 | 307 |
| 4072 | 401 | 100 | 212 | 212 | 212 | 308 |
| 4073 | 402 | 21 | 212 | 201 | 202 | 301 |
| 4074 | 401 | 91 | 201 | 202 | 201 | 305 |
| 4075 | 403 | 2 | 201 | 201 | 201 | 308 |
| 4076 | 401 | 71 | 211 | 212 | 202 | 306 |
| 4077 | 401 | 110 | 207 | 201 | 201 | 302 |
| 4078 | 407 | 85 | 204 | 201 | 212 | 301 |

Compounds having the formula:

TABLE 24

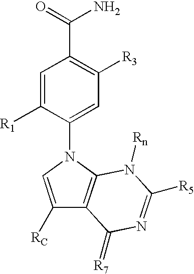

wherein R₁, R₃, R_{C}, Rₙ, R₅ and R₇ are listed in Table 24.

| Compound | | | | | | |
|---|---|---|---|---|---|---|
| No. | R₁ | R₃ | R_{C} | Rₙ | R₅ | R₇ |
| 4079 | 407 | 17 | 207 | 201 | 201 | 308 |
| 4080 | 404 | 33 | 203 | 202 | 202 | 302 |
| 4081 | 401 | 106 | 205 | 202 | 212 | 307 |
| 4082 | 404 | 88 | 211 | 201 | 201 | 306 |
| 4083 | 402 | 91 | 209 | 202 | 212 | 302 |
| 4084 | 402 | 75 | 204 | 202 | 202 | 302 |
| 4085 | 401 | 96 | 211 | 201 | 201 | 301 |
| 4086 | 401 | 92 | 204 | 201 | 212 | 301 |
| 4087 | 405 | 39 | 204 | 202 | 212 | 302 |
| 4088 | 404 | 80 | 204 | 202 | 212 | 306 |
| 4089 | 404 | 112 | 212 | 202 | 201 | 304 |
| 4090 | 406 | 97 | 210 | 202 | 201 | 304 |
| 4091 | 401 | 85 | 212 | 201 | 212 | 307 |
| 4092 | 401 | 98 | 208 | 212 | 201 | 301 |
| 4093 | 402 | 120 | 201 | 201 | 201 | 304 |
| 4094 | 401 | 108 | 205 | 201 | 212 | 302 |
| 4095 | 404 | 88 | 208 | 212 | 212 | 301 |
| 4096 | 402 | 96 | 201 | 202 | 201 | 304 |
| 4097 | 405 | 107 | 204 | 212 | 201 | 302 |
| 4098 | 402 | 128 | 212 | 201 | 201 | 303 |
| 4099 | 401 | 111 | 204 | 201 | 202 | 308 |
| 4100 | 405 | 34 | 205 | 202 | 201 | 304 |
| 4101 | 404 | 86 | 201 | 201 | 202 | 301 |
| 4102 | 401 | 85 | 204 | 212 | 202 | 301 |
| 4103 | 405 | 94 | 201 | 201 | 212 | 304 |
| 4104 | 406 | 86 | 212 | 212 | 212 | 301 |
| 4105 | 407 | 68 | 204 | 201 | 212 | 304 |
| 4106 | 401 | 58 | 202 | 202 | 212 | 306 |
| 4107 | 401 | 54 | 212 | 202 | 201 | 302 |
| 4108 | 403 | 109 | 201 | 202 | 212 | 301 |
| 4109 | 401 | 9 | 212 | 202 | 212 | 305 |
| 4110 | 401 | 66 | 201 | 201 | 212 | 304 |
| 4111 | 401 | 96 | 201 | 212 | 201 | 308 |
| 4112 | 401 | 86 | 201 | 202 | 212 | 302 |
| 4113 | 401 | 101 | 201 | 201 | 212 | 308 |
| 4114 | 401 | 84 | 203 | 202 | 212 | 303 |
| 4115 | 401 | 96 | 212 | 202 | 201 | 301 |
| 4116 | 401 | 109 | 207 | 202 | 201 | 302 |
| 4117 | 405 | 85 | 210 | 201 | 202 | 303 |
| 4118 | 401 | 20 | 201 | 201 | 201 | 305 |
| 4119 | 404 | 57 | 202 | 201 | 212 | 308 |
| 4120 | 402 | 60 | 202 | 201 | 201 | 301 |
| 4121 | 401 | 113 | 210 | 201 | 201 | 307 |
| 4122 | 405 | 109 | 207 | 202 | 201 | 304 |
| 4123 | 401 | 22 | 211 | 202 | 201 | 308 |
| 4124 | 403 | 89 | 206 | 201 | 202 | 306 |
| 4125 | 401 | 101 | 212 | 201 | 202 | 307 |
| 4126 | 401 | 109 | 207 | 201 | 201 | 306 |
| 4127 | 406 | 91 | 210 | 201 | 201 | 308 |
| 4128 | 401 | 91 | 212 | 201 | 201 | 304 |
| 4129 | 403 | 37 | 204 | 202 | 202 | 306 |
| 4130 | 405 | 114 | 212 | 202 | 202 | 304 |
| 4131 | 407 | 98 | 212 | 201 | 201 | 307 |
| 4132 | 403 | 46 | 204 | 202 | 202 | 308 |
| 4133 | 402 | 98 | 209 | 201 | 201 | 307 |
| 4134 | 407 | 73 | 210 | 202 | 202 | 308 |
| 4135 | 401 | 28 | 212 | 212 | 202 | 302 |
| 4136 | 403 | 129 | 211 | 202 | 202 | 302 |
| 4137 | 405 | 19 | 202 | 202 | 201 | 308 |
| 4138 | 401 | 79 | 206 | 201 | 212 | 301 |
| 4139 | 401 | 83 | 211 | 202 | 212 | 303 |
| 4140 | 402 | 103 | 205 | 202 | 201 | 301 |
| 4141 | 402 | 86 | 210 | 201 | 212 | 303 |
| 4142 | 404 | 51 | 211 | 201 | 201 | 307 |
| 4143 | 403 | 64 | 212 | 202 | 201 | 308 |
| 4144 | 407 | 65 | 212 | 201 | 212 | 306 |
| 4145 | 406 | 96 | 210 | 212 | 212 | 305 |
| 4146 | 403 | 8 | 204 | 201 | 212 | 301 |
| 4147 | 401 | 38 | 206 | 202 | 201 | 305 |
| 4148 | 401 | 72 | 204 | 201 | 202 | 305 |
| 4149 | 405 | 59 | 206 | 202 | 201 | 306 |
| 4150 | 406 | 29 | 210 | 212 | 202 | 306 |
| 4151 | 401 | 130 | 206 | 202 | 202 | 306 |
| 4152 | 405 | 104 | 210 | 201 | 202 | 307 |
| 4153 | 401 | 25 | 204 | 201 | 202 | 306 |
| 4154 | 407 | 98 | 204 | 201 | 212 | 301 |
| 4155 | 401 | 115 | 206 | 212 | 201 | 308 |
| 4156 | 405 | 44 | 202 | 212 | 201 | 307 |
| 4157 | 401 | 55 | 204 | 201 | 201 | 302 |
| 4158 | 406 | 81 | 210 | 212 | 212 | 301 |
| 4159 | 406 | 47 | 203 | 201 | 202 | 306 |
| 4160 | 401 | 88 | 204 | 201 | 202 | 306 |
| 4161 | 406 | 88 | 206 | 202 | 202 | 303 |
| 4162 | 406 | 96 | 204 | 201 | 201 | 308 |
| 4163 | 401 | 116 | 205 | 202 | 202 | 301 |
| 4164 | 407 | 90 | 205 | 201 | 212 | 305 |
| 4165 | 401 | 24 | 204 | 212 | 201 | 301 |
| 4166 | 401 | 76 | 202 | 212 | 202 | 306 |
| 4167 | 404 | 121 | 209 | 202 | 202 | 308 |
| 4168 | 401 | 93 | 205 | 201 | 212 | 307 |
| 4169 | 401 | 101 | 203 | 201 | 201 | 301 |
| 4170 | 402 | 91 | 203 | 212 | 201 | 306 |
| 4171 | 401 | 101 | 205 | 212 | 202 | 302 |
| 4172 | 404 | 32 | 211 | 202 | 212 | 301 |
| 4173 | 403 | 6 | 210 | 202 | 202 | 306 |
| 4174 | 404 | 109 | 204 | 202 | 201 | 303 |
| 4175 | 401 | 12 | 207 | 202 | 201 | 303 |
| 4176 | 405 | 101 | 207 | 202 | 212 | 301 |
| 4177 | 407 | 86 | 205 | 201 | 201 | 303 |
| 4178 | 402 | 18 | 202 | 201 | 202 | 306 |
| 4179 | 406 | 96 | 212 | 201 | 212 | 307 |
| 4180 | 401 | 82 | 209 | 212 | 202 | 302 |
| 4181 | 404 | 85 | 204 | 201 | 212 | 308 |
| 4182 | 401 | 127 | 210 | 201 | 201 | 301 |
| 4183 | 405 | 88 | 206 | 202 | 212 | 301 |
| 4184 | 402 | 63 | 210 | 201 | 202 | 308 |
| 4185 | 403 | 16 | 205 | 201 | 201 | 306 |
| 4186 | 401 | 11 | 212 | 202 | 212 | 306 |
| 4187 | 407 | 99 | 203 | 212 | 202 | 307 |
| 4188 | 402 | 31 | 208 | 201 | 212 | 305 |
| 4189 | 405 | 98 | 205 | 202 | 201 | 302 |
| 4190 | 405 | 3 | 203 | 201 | 202 | 302 |
| 4191 | 406 | 10 | 211 | 201 | 202 | 308 |
| 4192 | 403 | 87 | 206 | 212 | 202 | 302 |
| 4193 | 401 | 35 | 209 | 201 | 202 | 301 |
| 4194 | 405 | 26 | 209 | 202 | 212 | 303 |
| 4195 | 407 | 74 | 210 | 201 | 201 | 303 |
| 4196 | 407 | 36 | 212 | 202 | 201 | 301 |
| 4197 | 405 | 56 | 209 | 212 | 202 | 304 |
| 4198 | 403 | 88 | 209 | 201 | 212 | 307 |
| 4199 | 402 | 95 | 204 | 201 | 201 | 308 |
| 4200 | 406 | 96 | 209 | 202 | 201 | 302 |
| 4201 | 404 | 61 | 202 | 201 | 202 | 301 |
| 4202 | 401 | 77 | 204 | 201 | 212 | 301 |
| 4203 | 401 | 27 | 212 | 202 | 202 | 306 |
| 4204 | 403 | 105 | 212 | 201 | 202 | 306 |
| 4205 | 401 | 69 | 210 | 202 | 201 | 303 |
| 4206 | 401 | 41 | 202 | 202 | 202 | 302 |
| 4207 | 404 | 4 | 201 | 202 | 201 | 304 |
| 4208 | 401 | 7 | 204 | 202 | 201 | 307 |
| 4209 | 401 | 86 | 204 | 202 | 212 | 304 |
| 4210 | 401 | 23 | 205 | 202 | 202 | 301 |
| 4211 | 401 | 118 | 203 | 201 | 212 | 306 |
| 4212 | 401 | 101 | 211 | 201 | 201 | 306 |
| 4213 | 406 | 62 | 205 | 212 | 212 | 303 |
| 4214 | 401 | 78 | 204 | 201 | 201 | 307 |
| 4215 | 404 | 1 | 201 | 202 | 212 | 308 |
| 4216 | 401 | 70 | 205 | 212 | 201 | 302 |
| 4217 | 401 | 50 | 212 | 212 | 202 | 307 |
| 4218 | 407 | 91 | 206 | 201 | 212 | 307 |
| 4219 | 401 | 125 | 201 | 201 | 212 | 308 |
| 4220 | 403 | 102 | 201 | 201 | 201 | 308 |
| 4221 | 402 | 45 | 205 | 201 | 201 | 303 |
| 4222 | 401 | 88 | 210 | 212 | 202 | 306 |
| 4223 | 401 | 88 | 210 | 202 | 201 | 301 |
| 4224 | 401 | 85 | 211 | 212 | 212 | 301 |
| 4225 | 404 | 15 | 211 | 201 | 212 | 302 |
| 4226 | 407 | 67 | 211 | 201 | 201 | 302 |
| 4227 | 403 | 40 | 210 | 201 | 202 | 306 |
| 4228 | 407 | 30 | 210 | 201 | 201 | 305 |
| 4229 | 401 | 53 | 204 | 201 | 201 | 303 |
| 4230 | 401 | 48 | 212 | 201 | 202 | 303 |
| 4231 | 406 | 122 | 201 | 201 | 201 | 308 |
| 4232 | 401 | 123 | 201 | 201 | 201 | 307 |
| 4233 | 407 | 43 | 212 | 201 | 202 | 308 |
| 4234 | 404 | 109 | 212 | 212 | 201 | 305 |
| 4235 | 402 | 14 | 203 | 212 | 201 | 308 |
| 4236 | 401 | 13 | 204 | 212 | 201 | 302 |
| 4237 | 401 | 117 | 211 | 202 | 202 | 302 |
| 4238 | 406 | 5 | 205 | 201 | 201 | 302 |
| 4239 | 402 | 124 | 203 | 201 | 201 | 308 |
| 4240 | 401 | 98 | 208 | 202 | 201 | 308 |
| 4241 | 406 | 109 | 204 | 202 | 212 | 303 |
| 4242 | 401 | 52 | 201 | 201 | 202 | 306 |
| 4243 | 406 | 42 | 205 | 201 | 212 | 308 |
| 4244 | 407 | 126 | 212 | 202 | 202 | 307 |
| 4245 | 403 | 119 | 212 | 201 | 212 | 302 |
| 4246 | 403 | 49 | 209 | 202 | 201 | 307 |
| 4247 | 401 | 100 | 212 | 212 | 212 | 308 |
| 4248 | 402 | 21 | 212 | 201 | 202 | 301 |
| 4249 | 401 | 91 | 201 | 202 | 201 | 305 |
| 4250 | 403 | 2 | 201 | 201 | 201 | 308 |
| 4251 | 401 | 71 | 211 | 212 | 202 | 306 |
| 4252 | 401 | 110 | 207 | 201 | 201 | 302 |
| 4253 | 407 | 85 | 204 | 201 | 212 | 301 |

Compounds having the formula:

TABLE 25

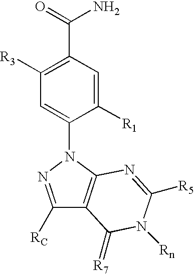

wherein R₁, R₃, R_{C}, Rₙ, R₅ and R₇ are listed in Table 25.

| Compound | | | | | | |
|---|---|---|---|---|---|---|
| No. | R₁ | R₃ | R_{C} | Rₙ | R₅ | R₇ |
| 4254 | 407 | 17 | 207 | 201 | 201 | 308 |
| 4255 | 404 | 33 | 203 | 202 | 202 | 302 |
| 4256 | 401 | 106 | 205 | 202 | 212 | 307 |
| 4257 | 404 | 88 | 211 | 201 | 201 | 306 |
| 4258 | 402 | 91 | 209 | 202 | 212 | 302 |
| 4259 | 402 | 75 | 204 | 202 | 202 | 302 |
| 4260 | 401 | 96 | 211 | 201 | 201 | 301 |
| 4261 | 401 | 92 | 204 | 201 | 212 | 301 |
| 4262 | 405 | 39 | 204 | 202 | 212 | 302 |
| 4263 | 404 | 80 | 204 | 202 | 212 | 306 |
| 4264 | 404 | 112 | 212 | 202 | 201 | 304 |
| 4265 | 406 | 97 | 210 | 202 | 201 | 304 |
| 4266 | 401 | 85 | 212 | 201 | 212 | 307 |
| 4267 | 401 | 98 | 208 | 212 | 201 | 301 |
| 4268 | 402 | 120 | 201 | 201 | 201 | 304 |
| 4269 | 401 | 108 | 205 | 201 | 212 | 302 |
| 4270 | 404 | 88 | 208 | 212 | 212 | 301 |
| 4271 | 402 | 96 | 201 | 202 | 201 | 304 |
| 4272 | 405 | 107 | 204 | 212 | 201 | 302 |
| 4273 | 402 | 128 | 212 | 201 | 201 | 303 |
| 4274 | 401 | 111 | 204 | 201 | 202 | 308 |
| 4275 | 405 | 34 | 205 | 202 | 201 | 304 |
| 4276 | 404 | 86 | 201 | 201 | 202 | 301 |
| 4277 | 401 | 85 | 204 | 212 | 202 | 301 |
| 4278 | 405 | 94 | 201 | 201 | 212 | 304 |
| 4279 | 406 | 86 | 212 | 212 | 212 | 301 |
| 4280 | 407 | 68 | 204 | 201 | 212 | 304 |
| 4281 | 401 | 58 | 202 | 202 | 212 | 306 |
| 4282 | 401 | 54 | 212 | 202 | 201 | 302 |
| 4283 | 403 | 109 | 201 | 202 | 212 | 301 |
| 4284 | 401 | 9 | 212 | 202 | 212 | 305 |
| 4285 | 401 | 66 | 201 | 201 | 212 | 304 |
| 4286 | 401 | 96 | 201 | 212 | 201 | 308 |
| 4287 | 401 | 86 | 201 | 202 | 212 | 302 |
| 4288 | 401 | 101 | 201 | 201 | 212 | 308 |
| 4289 | 401 | 84 | 203 | 202 | 212 | 303 |
| 4290 | 401 | 96 | 212 | 202 | 201 | 301 |
| 4291 | 401 | 109 | 207 | 202 | 201 | 302 |
| 4292 | 405 | 85 | 210 | 201 | 202 | 303 |
| 4293 | 401 | 20 | 201 | 201 | 201 | 305 |
| 4294 | 404 | 57 | 202 | 201 | 212 | 308 |
| 4295 | 402 | 60 | 202 | 201 | 201 | 301 |
| 4296 | 401 | 113 | 210 | 201 | 201 | 307 |
| 4297 | 405 | 109 | 207 | 202 | 201 | 304 |
| 4298 | 401 | 22 | 211 | 202 | 201 | 308 |
| 4299 | 403 | 89 | 206 | 201 | 202 | 306 |
| 4300 | 401 | 101 | 212 | 201 | 202 | 307 |
| 4301 | 401 | 109 | 207 | 201 | 201 | 306 |
| 4302 | 406 | 91 | 210 | 201 | 201 | 308 |
| 4303 | 401 | 91 | 212 | 201 | 201 | 304 |
| 4304 | 403 | 37 | 204 | 202 | 202 | 306 |
| 4305 | 405 | 114 | 212 | 202 | 202 | 304 |
| 4306 | 407 | 98 | 212 | 201 | 201 | 307 |
| 4307 | 403 | 46 | 204 | 202 | 202 | 308 |
| 4308 | 402 | 98 | 209 | 201 | 201 | 307 |
| 4309 | 407 | 73 | 210 | 202 | 202 | 308 |
| 4310 | 401 | 28 | 212 | 212 | 202 | 302 |
| 4311 | 403 | 129 | 211 | 202 | 202 | 302 |
| 4312 | 405 | 19 | 202 | 202 | 201 | 308 |
| 4313 | 401 | 79 | 206 | 201 | 212 | 301 |
| 4314 | 401 | 83 | 211 | 202 | 212 | 303 |
| 4315 | 402 | 103 | 205 | 202 | 201 | 301 |
| 4316 | 402 | 86 | 210 | 201 | 212 | 303 |
| 4317 | 404 | 51 | 211 | 201 | 201 | 307 |
| 4318 | 403 | 64 | 212 | 202 | 201 | 308 |
| 4319 | 407 | 65 | 212 | 201 | 212 | 306 |
| 4320 | 406 | 96 | 210 | 212 | 212 | 305 |
| 4321 | 403 | 8 | 204 | 201 | 212 | 301 |
| 4322 | 401 | 38 | 206 | 202 | 201 | 305 |
| 4323 | 401 | 72 | 204 | 201 | 202 | 305 |
| 4324 | 405 | 59 | 206 | 202 | 201 | 306 |
| 4325 | 406 | 29 | 210 | 212 | 202 | 306 |
| 4326 | 401 | 130 | 206 | 202 | 202 | 306 |
| 4327 | 405 | 104 | 210 | 201 | 202 | 307 |
| 4328 | 401 | 25 | 204 | 201 | 202 | 306 |
| 4329 | 407 | 98 | 204 | 201 | 212 | 301 |
| 4330 | 401 | 115 | 206 | 212 | 201 | 308 |
| 4331 | 405 | 44 | 202 | 212 | 201 | 307 |
| 4332 | 401 | 55 | 204 | 201 | 201 | 302 |
| 4333 | 406 | 81 | 210 | 212 | 212 | 301 |
| 4334 | 406 | 47 | 203 | 201 | 202 | 306 |
| 4335 | 401 | 88 | 204 | 201 | 202 | 306 |
| 4336 | 406 | 88 | 206 | 202 | 202 | 303 |
| 4337 | 406 | 96 | 204 | 201 | 201 | 308 |
| 4338 | 401 | 116 | 205 | 202 | 202 | 301 |
| 4339 | 407 | 90 | 205 | 201 | 212 | 305 |
| 4340 | 401 | 24 | 204 | 212 | 201 | 301 |
| 4341 | 401 | 76 | 202 | 212 | 202 | 306 |
| 4342 | 404 | 121 | 209 | 202 | 202 | 308 |
| 4343 | 401 | 93 | 205 | 201 | 212 | 307 |
| 4344 | 401 | 101 | 203 | 201 | 201 | 301 |
| 4345 | 402 | 91 | 203 | 212 | 201 | 306 |
| 4346 | 401 | 101 | 205 | 212 | 202 | 302 |
| 4347 | 404 | 32 | 211 | 202 | 212 | 301 |
| 4348 | 403 | 6 | 210 | 202 | 202 | 306 |
| 4349 | 404 | 109 | 204 | 202 | 201 | 303 |
| 4350 | 401 | 12 | 207 | 202 | 201 | 303 |
| 4351 | 405 | 101 | 207 | 202 | 212 | 301 |
| 4352 | 407 | 86 | 205 | 201 | 201 | 303 |
| 4353 | 402 | 18 | 202 | 201 | 202 | 306 |
| 4354 | 406 | 96 | 212 | 201 | 212 | 307 |
| 4355 | 401 | 82 | 209 | 212 | 202 | 302 |
| 4356 | 404 | 85 | 204 | 201 | 212 | 308 |
| 4357 | 401 | 127 | 210 | 201 | 201 | 301 |
| 4358 | 405 | 88 | 206 | 202 | 212 | 301 |
| 4359 | 402 | 63 | 210 | 201 | 202 | 308 |
| 4360 | 403 | 16 | 205 | 201 | 201 | 306 |
| 4361 | 401 | 11 | 212 | 202 | 212 | 306 |
| 4362 | 407 | 99 | 203 | 212 | 202 | 307 |
| 4363 | 402 | 31 | 208 | 201 | 212 | 305 |
| 4364 | 405 | 98 | 205 | 202 | 201 | 302 |
| 4365 | 405 | 3 | 203 | 201 | 202 | 302 |
| 4366 | 406 | 10 | 211 | 201 | 202 | 308 |
| 4367 | 403 | 87 | 206 | 212 | 202 | 302 |
| 4368 | 401 | 35 | 209 | 201 | 202 | 301 |
| 4369 | 405 | 26 | 209 | 202 | 212 | 303 |
| 4370 | 407 | 74 | 210 | 201 | 201 | 303 |
| 4371 | 407 | 36 | 212 | 202 | 201 | 301 |
| 4372 | 405 | 56 | 209 | 212 | 202 | 304 |
| 4373 | 403 | 88 | 209 | 201 | 212 | 307 |
| 4374 | 402 | 95 | 204 | 201 | 201 | 308 |
| 4375 | 406 | 96 | 209 | 202 | 201 | 302 |
| 4376 | 404 | 61 | 202 | 201 | 202 | 301 |
| 4377 | 401 | 77 | 204 | 201 | 212 | 301 |
| 4378 | 401 | 27 | 212 | 202 | 202 | 306 |
| 4379 | 403 | 105 | 212 | 201 | 202 | 306 |
| 4380 | 401 | 69 | 210 | 202 | 201 | 303 |
| 4381 | 401 | 41 | 202 | 202 | 202 | 302 |
| 4382 | 404 | 4 | 201 | 202 | 201 | 304 |
| 4383 | 401 | 7 | 204 | 202 | 201 | 307 |
| 4384 | 401 | 86 | 204 | 202 | 212 | 304 |
| 4385 | 401 | 23 | 205 | 202 | 202 | 301 |
| 4386 | 401 | 118 | 203 | 201 | 212 | 306 |
| 4387 | 401 | 101 | 211 | 201 | 201 | 306 |
| 4388 | 406 | 62 | 205 | 212 | 212 | 303 |
| 4389 | 401 | 78 | 204 | 201 | 201 | 307 |
| 4390 | 404 | 1 | 201 | 202 | 212 | 308 |
| 4391 | 401 | 70 | 205 | 212 | 201 | 302 |
| 4392 | 401 | 50 | 212 | 212 | 202 | 307 |
| 4393 | 407 | 91 | 206 | 201 | 212 | 307 |
| 4394 | 401 | 125 | 201 | 201 | 212 | 308 |
| 4395 | 403 | 102 | 201 | 201 | 201 | 308 |
| 4396 | 402 | 45 | 205 | 201 | 201 | 303 |
| 4397 | 401 | 88 | 210 | 212 | 202 | 306 |
| 4398 | 401 | 88 | 210 | 202 | 201 | 301 |
| 4399 | 401 | 85 | 211 | 212 | 212 | 301 |
| 4400 | 404 | 15 | 211 | 201 | 212 | 302 |
| 4401 | 407 | 67 | 211 | 201 | 201 | 302 |
| 4402 | 403 | 40 | 210 | 201 | 202 | 306 |
| 4403 | 407 | 30 | 210 | 201 | 201 | 305 |
| 4404 | 401 | 53 | 204 | 201 | 201 | 303 |
| 4405 | 401 | 48 | 212 | 201 | 202 | 303 |
| 4406 | 406 | 122 | 201 | 201 | 201 | 308 |
| 4407 | 401 | 123 | 201 | 201 | 201 | 307 |
| 4408 | 407 | 43 | 212 | 201 | 202 | 308 |
| 4409 | 404 | 109 | 212 | 212 | 201 | 305 |
| 4410 | 402 | 14 | 203 | 212 | 201 | 308 |
| 4411 | 401 | 13 | 204 | 212 | 201 | 302 |
| 4412 | 401 | 117 | 211 | 202 | 202 | 302 |
| 4413 | 406 | 5 | 205 | 201 | 201 | 302 |
| 4414 | 402 | 124 | 203 | 201 | 201 | 308 |
| 4415 | 401 | 98 | 208 | 202 | 201 | 308 |
| 4416 | 406 | 109 | 204 | 202 | 212 | 303 |
| 4417 | 401 | 52 | 201 | 201 | 202 | 306 |
| 4418 | 406 | 42 | 205 | 201 | 212 | 308 |
| 4419 | 407 | 126 | 212 | 202 | 202 | 307 |
| 4420 | 403 | 119 | 212 | 201 | 212 | 302 |
| 4421 | 403 | 49 | 209 | 202 | 201 | 307 |
| 4422 | 401 | 100 | 212 | 212 | 212 | 308 |
| 4423 | 402 | 21 | 212 | 201 | 202 | 301 |
| 4424 | 401 | 91 | 201 | 202 | 201 | 305 |
| 4425 | 403 | 2 | 201 | 201 | 201 | 308 |
| 4426 | 401 | 71 | 211 | 212 | 202 | 306 |
| 4427 | 401 | 110 | 207 | 201 | 201 | 302 |
| 4428 | 407 | 85 | 204 | 201 | 212 | 301 |

Compounds having the formula:

TABLE 26

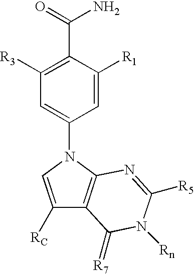

wherein R₁, R₃, R_{C}, Rₙ, R₅ and R₇ are listed in Table 26.

| Compound | | | | | | |
|---|---|---|---|---|---|---|
| No. | R₁ | R₃ | R_{C} | Rₙ | R₅ | R₇ |
| 4429 | 407 | 17 | 207 | 201 | 201 | 308 |
| 4430 | 404 | 33 | 203 | 202 | 202 | 302 |
| 4431 | 401 | 106 | 205 | 202 | 212 | 307 |
| 4432 | 404 | 88 | 211 | 201 | 201 | 306 |
| 4433 | 402 | 91 | 209 | 202 | 212 | 302 |
| 4434 | 402 | 75 | 204 | 202 | 202 | 302 |
| 4435 | 401 | 96 | 211 | 201 | 201 | 301 |
| 4436 | 401 | 92 | 204 | 201 | 212 | 301 |
| 4437 | 405 | 39 | 204 | 202 | 212 | 302 |
| 4438 | 404 | 80 | 204 | 202 | 212 | 306 |
| 4439 | 404 | 112 | 212 | 202 | 201 | 304 |
| 4440 | 406 | 97 | 210 | 202 | 201 | 304 |
| 4441 | 401 | 85 | 212 | 201 | 212 | 307 |
| 4442 | 401 | 98 | 208 | 212 | 201 | 301 |
| 4443 | 402 | 120 | 201 | 201 | 201 | 304 |
| 4444 | 401 | 108 | 205 | 201 | 212 | 302 |
| 4445 | 404 | 88 | 208 | 212 | 212 | 301 |
| 4446 | 402 | 96 | 201 | 202 | 201 | 304 |
| 4447 | 405 | 107 | 204 | 212 | 201 | 302 |
| 4448 | 402 | 128 | 212 | 201 | 201 | 303 |
| 4449 | 401 | 111 | 204 | 201 | 202 | 308 |
| 4450 | 405 | 34 | 205 | 202 | 201 | 304 |
| 4451 | 404 | 86 | 201 | 201 | 202 | 301 |
| 4452 | 401 | 85 | 204 | 212 | 202 | 301 |
| 4453 | 405 | 94 | 201 | 201 | 212 | 304 |
| 4454 | 406 | 86 | 212 | 212 | 212 | 301 |
| 4455 | 407 | 68 | 204 | 201 | 212 | 304 |
| 4456 | 401 | 58 | 202 | 202 | 212 | 306 |
| 4457 | 401 | 54 | 212 | 202 | 201 | 302 |
| 4458 | 403 | 109 | 201 | 202 | 212 | 301 |
| 4459 | 401 | 9 | 212 | 202 | 212 | 305 |
| 4460 | 401 | 66 | 201 | 201 | 212 | 304 |
| 4461 | 401 | 96 | 201 | 212 | 201 | 308 |
| 4462 | 401 | 86 | 201 | 202 | 212 | 302 |
| 4463 | 401 | 101 | 201 | 201 | 212 | 308 |
| 4464 | 401 | 84 | 203 | 202 | 212 | 303 |
| 4465 | 401 | 96 | 212 | 202 | 201 | 301 |
| 4466 | 401 | 109 | 207 | 202 | 201 | 302 |
| 4467 | 405 | 85 | 210 | 201 | 202 | 303 |
| 4468 | 401 | 20 | 201 | 201 | 201 | 305 |
| 4469 | 404 | 57 | 202 | 201 | 212 | 308 |
| 4470 | 402 | 60 | 202 | 201 | 201 | 301 |
| 4471 | 401 | 113 | 210 | 201 | 201 | 307 |
| 4472 | 405 | 109 | 207 | 202 | 201 | 304 |
| 4473 | 401 | 22 | 211 | 202 | 201 | 308 |
| 4474 | 403 | 89 | 206 | 201 | 202 | 306 |
| 4475 | 401 | 101 | 212 | 201 | 202 | 307 |
| 4476 | 401 | 109 | 207 | 201 | 201 | 306 |
| 4477 | 406 | 91 | 210 | 201 | 201 | 308 |
| 4478 | 401 | 91 | 212 | 201 | 201 | 304 |
| 4479 | 403 | 37 | 204 | 202 | 202 | 306 |
| 4480 | 405 | 114 | 212 | 202 | 202 | 304 |
| 4481 | 407 | 98 | 212 | 201 | 201 | 307 |
| 4482 | 403 | 46 | 204 | 202 | 202 | 308 |
| 4483 | 402 | 98 | 209 | 201 | 201 | 307 |
| 4484 | 407 | 73 | 210 | 202 | 202 | 308 |
| 4485 | 401 | 28 | 212 | 212 | 202 | 302 |
| 4486 | 403 | 129 | 211 | 202 | 202 | 302 |
| 4487 | 405 | 19 | 202 | 202 | 201 | 308 |
| 4488 | 401 | 79 | 206 | 201 | 212 | 301 |
| 4489 | 401 | 83 | 211 | 202 | 212 | 303 |
| 4490 | 402 | 103 | 205 | 202 | 201 | 301 |
| 4491 | 402 | 86 | 210 | 201 | 212 | 303 |
| 4492 | 404 | 51 | 211 | 201 | 201 | 307 |
| 4493 | 403 | 64 | 212 | 202 | 201 | 308 |
| 4494 | 407 | 65 | 212 | 201 | 212 | 306 |
| 4495 | 406 | 96 | 210 | 212 | 212 | 305 |
| 4496 | 403 | 8 | 204 | 201 | 212 | 301 |
| 4497 | 401 | 38 | 206 | 202 | 201 | 305 |
| 4498 | 401 | 72 | 204 | 201 | 202 | 305 |
| 4499 | 405 | 59 | 206 | 202 | 201 | 306 |
| 4500 | 406 | 29 | 210 | 212 | 202 | 306 |
| 4501 | 401 | 130 | 206 | 202 | 202 | 306 |
| 4502 | 405 | 104 | 210 | 201 | 202 | 307 |
| 4503 | 401 | 25 | 204 | 201 | 202 | 306 |
| 4504 | 407 | 98 | 204 | 201 | 212 | 301 |
| 4505 | 401 | 115 | 206 | 212 | 201 | 308 |
| 4506 | 405 | 44 | 202 | 212 | 201 | 307 |
| 4507 | 401 | 55 | 204 | 201 | 201 | 302 |
| 4508 | 406 | 81 | 210 | 212 | 212 | 301 |
| 4509 | 406 | 47 | 203 | 201 | 202 | 306 |
| 4510 | 401 | 88 | 204 | 201 | 202 | 306 |
| 4511 | 406 | 88 | 206 | 202 | 202 | 303 |
| 4512 | 406 | 96 | 204 | 201 | 201 | 308 |
| 4513 | 401 | 116 | 205 | 202 | 202 | 301 |
| 4514 | 407 | 90 | 205 | 201 | 212 | 305 |
| 4515 | 401 | 24 | 204 | 212 | 201 | 301 |
| 4516 | 401 | 76 | 202 | 212 | 202 | 306 |
| 4517 | 404 | 121 | 209 | 202 | 202 | 308 |
| 4518 | 401 | 93 | 205 | 201 | 212 | 307 |
| 4519 | 401 | 101 | 203 | 201 | 201 | 301 |
| 4520 | 402 | 91 | 203 | 212 | 201 | 306 |
| 4521 | 401 | 101 | 205 | 212 | 202 | 302 |
| 4522 | 404 | 32 | 211 | 202 | 212 | 301 |
| 4523 | 403 | 6 | 210 | 202 | 202 | 306 |
| 4524 | 404 | 109 | 204 | 202 | 201 | 303 |
| 4525 | 401 | 12 | 207 | 202 | 201 | 303 |
| 4526 | 405 | 101 | 207 | 202 | 212 | 301 |
| 4527 | 407 | 86 | 205 | 201 | 201 | 303 |
| 4528 | 402 | 18 | 202 | 201 | 202 | 306 |
| 4529 | 406 | 96 | 212 | 201 | 212 | 307 |
| 4530 | 401 | 82 | 209 | 212 | 202 | 302 |
| 4531 | 404 | 85 | 204 | 201 | 212 | 308 |
| 4532 | 401 | 127 | 210 | 201 | 201 | 301 |
| 4533 | 405 | 88 | 206 | 202 | 212 | 301 |
| 4534 | 402 | 63 | 210 | 201 | 202 | 308 |
| 4535 | 403 | 16 | 205 | 201 | 201 | 306 |
| 4536 | 401 | 11 | 212 | 202 | 212 | 306 |
| 4537 | 407 | 99 | 203 | 212 | 202 | 307 |
| 4538 | 402 | 31 | 208 | 201 | 212 | 305 |
| 4539 | 405 | 98 | 205 | 202 | 201 | 302 |
| 4540 | 405 | 3 | 203 | 201 | 202 | 302 |
| 4541 | 406 | 10 | 211 | 201 | 202 | 308 |
| 4542 | 403 | 87 | 206 | 212 | 202 | 302 |
| 4543 | 401 | 35 | 209 | 201 | 202 | 301 |
| 4544 | 405 | 26 | 209 | 202 | 212 | 303 |
| 4545 | 407 | 74 | 210 | 201 | 201 | 303 |
| 4546 | 407 | 36 | 212 | 202 | 201 | 301 |
| 4547 | 405 | 56 | 209 | 212 | 202 | 304 |
| 4548 | 403 | 88 | 209 | 201 | 212 | 307 |
| 4549 | 402 | 95 | 204 | 201 | 201 | 308 |
| 4550 | 406 | 96 | 209 | 202 | 201 | 302 |
| 4551 | 404 | 61 | 202 | 201 | 202 | 301 |
| 4552 | 401 | 77 | 204 | 201 | 212 | 301 |
| 4553 | 401 | 27 | 212 | 202 | 202 | 306 |
| 4554 | 403 | 105 | 212 | 201 | 202 | 306 |
| 4555 | 401 | 69 | 210 | 202 | 201 | 303 |
| 4556 | 401 | 41 | 202 | 202 | 202 | 302 |
| 4557 | 404 | 4 | 201 | 202 | 201 | 304 |
| 4558 | 401 | 7 | 204 | 202 | 201 | 307 |
| 4559 | 401 | 86 | 204 | 202 | 212 | 304 |
| 4560 | 401 | 23 | 205 | 202 | 202 | 301 |
| 4561 | 401 | 118 | 203 | 201 | 212 | 306 |
| 4562 | 401 | 101 | 211 | 201 | 201 | 306 |
| 4563 | 406 | 62 | 205 | 212 | 212 | 303 |
| 4564 | 401 | 78 | 204 | 201 | 201 | 307 |
| 4565 | 404 | 1 | 201 | 202 | 212 | 308 |
| 4566 | 401 | 70 | 205 | 212 | 201 | 302 |
| 4567 | 401 | 50 | 212 | 212 | 202 | 307 |
| 4568 | 407 | 91 | 206 | 201 | 212 | 307 |
| 4569 | 401 | 125 | 201 | 201 | 212 | 308 |
| 4570 | 403 | 102 | 201 | 201 | 201 | 308 |
| 4571 | 402 | 45 | 205 | 201 | 201 | 303 |
| 4572 | 401 | 88 | 210 | 212 | 202 | 306 |
| 4573 | 401 | 88 | 210 | 202 | 201 | 301 |
| 4574 | 401 | 85 | 211 | 212 | 212 | 301 |
| 4575 | 404 | 15 | 211 | 201 | 212 | 302 |
| 4576 | 407 | 67 | 211 | 201 | 201 | 302 |
| 4577 | 403 | 40 | 210 | 201 | 202 | 306 |
| 4578 | 407 | 30 | 210 | 201 | 201 | 305 |
| 4579 | 401 | 53 | 204 | 201 | 201 | 303 |
| 4580 | 401 | 48 | 212 | 201 | 202 | 303 |
| 4581 | 406 | 122 | 201 | 201 | 201 | 308 |
| 4582 | 401 | 123 | 201 | 201 | 201 | 307 |
| 4583 | 407 | 43 | 212 | 201 | 202 | 308 |
| 4584 | 404 | 109 | 212 | 212 | 201 | 305 |
| 4585 | 402 | 14 | 203 | 212 | 201 | 308 |
| 4586 | 401 | 13 | 204 | 212 | 201 | 302 |
| 4587 | 401 | 117 | 211 | 202 | 202 | 302 |
| 4588 | 406 | 5 | 205 | 201 | 201 | 302 |
| 4589 | 402 | 124 | 203 | 201 | 201 | 308 |
| 4590 | 401 | 98 | 208 | 202 | 201 | 308 |
| 4591 | 406 | 109 | 204 | 202 | 212 | 303 |
| 4592 | 401 | 52 | 201 | 201 | 202 | 306 |
| 4593 | 406 | 42 | 205 | 201 | 212 | 308 |
| 4594 | 407 | 126 | 212 | 202 | 202 | 307 |
| 4595 | 403 | 119 | 212 | 201 | 212 | 302 |
| 4596 | 403 | 49 | 209 | 202 | 201 | 307 |
| 4597 | 401 | 100 | 212 | 212 | 212 | 308 |
| 4598 | 402 | 21 | 212 | 201 | 202 | 301 |
| 4599 | 401 | 91 | 201 | 202 | 201 | 305 |
| 4600 | 403 | 2 | 201 | 201 | 201 | 308 |
| 4601 | 401 | 71 | 211 | 212 | 202 | 306 |
| 4602 | 401 | 110 | 207 | 201 | 201 | 302 |
| 4603 | 407 | 85 | 204 | 201 | 212 | 301 |

Compounds having the formula:

TABLE 27

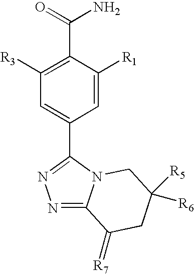

wherein R₁, R₃, R₅, R₆, and R₇ are defined in Table 27:

| Compound | | | | | |
|---|---|---|---|---|---|
| No. | R₁ | R₃ | R₅ | R₆ | R₇ |
| 4604 | 405 | 98 | 201 | 201 | 307 |
| 4605 | 407 | 115 | 201 | 201 | 308 |
| 4606 | 406 | 85 | 202 | 212 | 301 |
| 4607 | 405 | 98 | 201 | 201 | 308 |
| 4608 | 402 | 19 | 201 | 201 | 308 |
| 4609 | 401 | 88 | 202 | 212 | 303 |
| 4610 | 403 | 96 | 201 | 201 | 308 |
| 4611 | 405 | 109 | 201 | 201 | 305 |
| 4612 | 404 | 75 | 202 | 212 | 302 |
| 4613 | 401 | 84 | 212 | 212 | 303 |
| 4614 | 406 | 57 | 212 | 212 | 308 |
| 4615 | 405 | 46 | 202 | 212 | 308 |
| 4616 | 402 | 123 | 201 | 201 | 307 |
| 4617 | 403 | 29 | 202 | 212 | 306 |
| 4618 | 401 | 101 | 212 | 212 | 308 |
| 4619 | 401 | 94 | 212 | 212 | 304 |
| 4620 | 403 | 116 | 202 | 212 | 301 |
| 4621 | 401 | 106 | 212 | 212 | 307 |
| 4622 | 403 | 39 | 212 | 212 | 302 |
| 4623 | 403 | 44 | 201 | 201 | 307 |
| 4624 | 405 | 30 | 201 | 201 | 305 |
| 4625 | 401 | 70 | 201 | 201 | 302 |
| 4626 | 404 | 88 | 201 | 201 | 301 |
| 4627 | 401 | 88 | 201 | 201 | 306 |
| 4628 | 401 | 98 | 201 | 201 | 307 |
| 4629 | 401 | 118 | 212 | 212 | 306 |
| 4630 | 404 | 91 | 201 | 201 | 308 |
| 4631 | 407 | 76 | 202 | 212 | 306 |
| 4632 | 401 | 129 | 202 | 212 | 302 |
| 4633 | 401 | 95 | 201 | 201 | 308 |
| 4634 | 401 | 85 | 202 | 212 | 303 |
| 4635 | 405 | 78 | 201 | 201 | 307 |
| 4636 | 407 | 67 | 201 | 201 | 302 |
| 4637 | 406 | 17 | 201 | 201 | 308 |
| 4638 | 401 | 68 | 212 | 212 | 304 |
| 4639 | 401 | 43 | 202 | 212 | 308 |
| 4640 | 406 | 97 | 201 | 201 | 304 |
| 4641 | 405 | 111 | 202 | 212 | 308 |
| 4642 | 405 | 107 | 201 | 201 | 302 |
| 4643 | 401 | 32 | 212 | 212 | 301 |
| 4644 | 406 | 86 | 212 | 212 | 304 |
| 4645 | 403 | 88 | 202 | 212 | 306 |
| 4646 | 407 | 110 | 201 | 201 | 302 |
| 4647 | 405 | 25 | 202 | 212 | 306 |
| 4648 | 404 | 86 | 202 | 212 | 301 |
| 4649 | 401 | 96 | 201 | 201 | 301 |
| 4650 | 407 | 113 | 201 | 201 | 307 |
| 4651 | 404 | 55 | 201 | 201 | 302 |
| 4652 | 407 | 63 | 202 | 212 | 308 |
| 4653 | 401 | 45 | 201 | 201 | 303 |
| 4654 | 407 | 91 | 212 | 212 | 307 |
| 4655 | 402 | 2 | 201 | 201 | 308 |
| 4656 | 403 | 49 | 201 | 201 | 307 |
| 4657 | 401 | 101 | 201 | 201 | 301 |
| 4658 | 401 | 85 | 212 | 212 | 307 |
| 4659 | 401 | 28 | 202 | 212 | 302 |
| 4660 | 404 | 109 | 201 | 201 | 303 |
| 4661 | 401 | 91 | 212 | 212 | 302 |
| 4662 | 401 | 103 | 201 | 201 | 301 |
| 4663 | 405 | 112 | 201 | 201 | 304 |
| 4664 | 406 | 88 | 212 | 212 | 301 |
| 4665 | 406 | 96 | 201 | 201 | 302 |
| 4666 | 406 | 61 | 202 | 212 | 301 |
| 4667 | 401 | 109 | 212 | 212 | 301 |
| 4668 | 406 | 42 | 212 | 212 | 308 |
| 4669 | 401 | 27 | 202 | 212 | 306 |
| 4670 | 401 | 7 | 201 | 201 | 307 |
| 4671 | 401 | 87 | 202 | 212 | 302 |
| 4672 | 404 | 96 | 201 | 201 | 304 |
| 4673 | 402 | 37 | 202 | 212 | 306 |
| 4674 | 401 | 47 | 202 | 212 | 306 |
| 4675 | 406 | 31 | 212 | 212 | 305 |
| 4676 | 403 | 21 | 202 | 212 | 301 |
| 4677 | 407 | 71 | 202 | 212 | 306 |
| 4678 | 402 | 88 | 212 | 212 | 307 |
| 4679 | 401 | 96 | 201 | 201 | 301 |
| 4680 | 403 | 5 | 201 | 201 | 302 |
| 4681 | 406 | 4 | 201 | 201 | 304 |
| 4682 | 405 | 38 | 201 | 201 | 305 |
| 4683 | 407 | 34 | 201 | 201 | 304 |
| 4684 | 405 | 119 | 212 | 212 | 302 |
| 4685 | 401 | 72 | 202 | 212 | 305 |
| 4686 | 402 | 48 | 202 | 212 | 303 |
| 4687 | 401 | 14 | 201 | 201 | 308 |
| 4688 | 401 | 124 | 201 | 201 | 308 |
| 4689 | 407 | 54 | 201 | 201 | 302 |
| 4690 | 404 | 90 | 212 | 212 | 305 |
| 4691 | 401 | 36 | 201 | 201 | 301 |
| 4692 | 406 | 83 | 212 | 212 | 303 |
| 4693 | 401 | 33 | 202 | 212 | 302 |
| 4694 | 404 | 101 | 212 | 212 | 301 |
| 4695 | 401 | 80 | 212 | 212 | 306 |
| 4696 | 402 | 109 | 201 | 201 | 302 |
| 4697 | 401 | 66 | 212 | 212 | 304 |
| 4698 | 401 | 85 | 212 | 212 | 301 |
| 4699 | 401 | 40 | 202 | 212 | 306 |
| 4700 | 406 | 96 | 212 | 212 | 305 |
| 4701 | 405 | 86 | 212 | 212 | 303 |
| 4702 | 402 | 65 | 212 | 212 | 306 |
| 4703 | 404 | 91 | 201 | 201 | 304 |
| 4704 | 404 | 101 | 202 | 212 | 302 |
| 4705 | 404 | 8 | 212 | 212 | 301 |
| 4706 | 401 | 89 | 202 | 212 | 306 |
| 4707 | 402 | 85 | 212 | 212 | 308 |
| 4708 | 405 | 128 | 201 | 201 | 303 |
| 4709 | 401 | 98 | 201 | 201 | 301 |
| 4710 | 402 | 11 | 212 | 212 | 306 |
| 4711 | 407 | 1 | 212 | 212 | 308 |
| 4712 | 406 | 88 | 202 | 212 | 306 |
| 4713 | 401 | 15 | 212 | 212 | 302 |
| 4714 | 402 | 98 | 201 | 201 | 302 |
| 4715 | 404 | 127 | 201 | 201 | 301 |
| 4716 | 404 | 117 | 202 | 212 | 302 |
| 4717 | 401 | 109 | 201 | 201 | 306 |
| 4718 | 405 | 91 | 201 | 201 | 305 |
| 4719 | 403 | 102 | 201 | 201 | 308 |
| 4720 | 401 | 53 | 201 | 201 | 303 |
| 4721 | 407 | 104 | 202 | 212 | 307 |
| 4722 | 407 | 126 | 202 | 212 | 307 |
| 4723 | 401 | 60 | 201 | 201 | 301 |
| 4724 | 407 | 86 | 212 | 212 | 302 |
| 4725 | 401 | 92 | 212 | 212 | 301 |
| 4726 | 402 | 64 | 201 | 201 | 308 |
| 4727 | 401 | 101 | 201 | 201 | 306 |
| 4728 | 401 | 74 | 201 | 201 | 303 |
| 4729 | 402 | 20 | 201 | 201 | 305 |
| 4730 | 402 | 121 | 202 | 212 | 308 |
| 4731 | 406 | 13 | 201 | 201 | 302 |
| 4732 | 401 | 23 | 202 | 212 | 301 |
| 4733 | 401 | 85 | 212 | 212 | 301 |
| 4734 | 401 | 18 | 202 | 212 | 306 |
| 4735 | 401 | 41 | 202 | 212 | 302 |
| 4736 | 402 | 52 | 202 | 212 | 306 |
| 4737 | 401 | 101 | 202 | 212 | 307 |
| 4738 | 403 | 82 | 202 | 212 | 302 |
| 4739 | 404 | 88 | 212 | 212 | 301 |
| 4740 | 401 | 24 | 201 | 201 | 301 |
| 4741 | 401 | 10 | 202 | 212 | 308 |
| 4742 | 403 | 100 | 212 | 212 | 308 |
| 4743 | 403 | 93 | 212 | 212 | 307 |
| 4744 | 401 | 12 | 201 | 201 | 303 |
| 4745 | 402 | 50 | 202 | 212 | 307 |
| 4746 | 404 | 120 | 201 | 201 | 304 |
| 4747 | 401 | 86 | 212 | 212 | 301 |
| 4748 | 405 | 99 | 202 | 212 | 307 |
| 4749 | 402 | 22 | 201 | 201 | 308 |
| 4750 | 402 | 58 | 212 | 212 | 306 |
| 4751 | 401 | 62 | 212 | 212 | 303 |
| 4752 | 407 | 125 | 212 | 212 | 308 |
| 4753 | 401 | 73 | 202 | 212 | 308 |
| 4754 | 401 | 3 | 202 | 212 | 302 |
| 4755 | 405 | 51 | 201 | 201 | 307 |
| 4756 | 401 | 109 | 212 | 212 | 303 |
| 4757 | 403 | 6 | 202 | 212 | 306 |
| 4758 | 407 | 81 | 212 | 212 | 301 |
| 4759 | 406 | 109 | 201 | 201 | 304 |
| 4760 | 401 | 105 | 202 | 212 | 306 |
| 4761 | 401 | 96 | 212 | 212 | 307 |
| 4762 | 401 | 16 | 201 | 201 | 306 |
| 4763 | 401 | 69 | 201 | 201 | 303 |
| 4764 | 401 | 114 | 202 | 212 | 304 |
| 4765 | 401 | 9 | 212 | 212 | 305 |
| 4766 | 401 | 108 | 212 | 212 | 302 |
| 4767 | 403 | 59 | 201 | 201 | 306 |
| 4768 | 401 | 91 | 201 | 201 | 306 |
| 4769 | 404 | 98 | 212 | 212 | 301 |
| 4770 | 401 | 77 | 212 | 212 | 301 |
| 4771 | 401 | 86 | 201 | 201 | 303 |
| 4772 | 407 | 35 | 202 | 212 | 301 |
| 4773 | 406 | 96 | 201 | 201 | 308 |
| 4774 | 401 | 130 | 202 | 212 | 306 |
| 4775 | 403 | 56 | 202 | 212 | 304 |
| 4776 | 403 | 79 | 212 | 212 | 301 |
| 4777 | 401 | 26 | 212 | 212 | 303 |
| 4778 | 401 | 122 | 201 | 201 | 308 |

Compounds having the formula:

TABLE 28

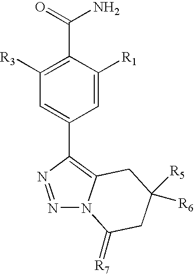

wherein R₁, R₃, R₅, R₆, and R₇ are defined in Table 28:

| Compound | | | | | |
|---|---|---|---|---|---|
| No. | R₁ | R₃ | R₅ | R₆ | R₇ |
| 4779 | 401 | 2 | 201 | 201 | 308 |
| 4780 | 402 | 87 | 202 | 212 | 302 |
| 4781 | 404 | 127 | 201 | 201 | 301 |
| 4782 | 407 | 98 | 201 | 201 | 308 |
| 4783 | 401 | 13 | 201 | 201 | 302 |
| 4784 | 401 | 39 | 212 | 212 | 302 |
| 4785 | 407 | 109 | 201 | 201 | 305 |
| 4786 | 407 | 9 | 212 | 212 | 305 |
| 4787 | 406 | 54 | 201 | 201 | 302 |
| 4788 | 401 | 85 | 202 | 212 | 303 |
| 4789 | 403 | 125 | 212 | 212 | 308 |
| 4790 | 406 | 116 | 202 | 212 | 301 |
| 4791 | 401 | 67 | 201 | 201 | 302 |
| 4792 | 401 | 121 | 202 | 212 | 308 |
| 4793 | 402 | 106 | 212 | 212 | 307 |
| 4794 | 401 | 98 | 201 | 201 | 301 |
| 4795 | 401 | 33 | 202 | 212 | 302 |
| 4796 | 401 | 92 | 212 | 212 | 301 |
| 4797 | 401 | 48 | 202 | 212 | 303 |
| 4798 | 405 | 85 | 212 | 212 | 301 |
| 4799 | 406 | 90 | 212 | 212 | 305 |
| 4800 | 403 | 101 | 212 | 212 | 301 |
| 4801 | 404 | 86 | 212 | 212 | 304 |
| 4802 | 401 | 51 | 201 | 201 | 307 |
| 4803 | 407 | 59 | 201 | 201 | 306 |
| 4804 | 404 | 47 | 202 | 212 | 306 |
| 4805 | 401 | 120 | 201 | 201 | 304 |
| 4806 | 403 | 3 | 202 | 212 | 302 |
| 4807 | 401 | 110 | 201 | 201 | 302 |
| 4808 | 405 | 91 | 201 | 201 | 306 |
| 4809 | 405 | 86 | 201 | 201 | 303 |
| 4810 | 404 | 25 | 202 | 212 | 306 |
| 4811 | 404 | 86 | 202 | 212 | 301 |
| 4812 | 401 | 86 | 212 | 212 | 301 |
| 4813 | 403 | 101 | 202 | 212 | 302 |
| 4814 | 406 | 16 | 201 | 201 | 306 |
| 4815 | 401 | 52 | 202 | 212 | 306 |
| 4816 | 404 | 36 | 201 | 201 | 301 |
| 4817 | 401 | 10 | 202 | 212 | 308 |
| 4818 | 404 | 18 | 202 | 212 | 306 |
| 4819 | 406 | 94 | 212 | 212 | 304 |
| 4820 | 405 | 79 | 212 | 212 | 301 |
| 4821 | 401 | 118 | 212 | 212 | 306 |
| 4822 | 405 | 115 | 201 | 201 | 308 |
| 4823 | 402 | 107 | 201 | 201 | 302 |
| 4824 | 401 | 78 | 201 | 201 | 307 |
| 4825 | 402 | 38 | 201 | 201 | 305 |
| 4826 | 401 | 60 | 201 | 201 | 301 |
| 4827 | 407 | 91 | 201 | 201 | 305 |
| 4828 | 401 | 99 | 202 | 212 | 307 |
| 4829 | 401 | 112 | 201 | 201 | 304 |
| 4830 | 406 | 109 | 212 | 212 | 303 |
| 4831 | 401 | 98 | 201 | 201 | 302 |
| 4832 | 402 | 88 | 212 | 212 | 301 |
| 4833 | 403 | 101 | 202 | 212 | 307 |
| 4834 | 401 | 50 | 202 | 212 | 307 |
| 4835 | 401 | 56 | 202 | 212 | 304 |
| 4836 | 406 | 66 | 212 | 212 | 304 |
| 4837 | 401 | 49 | 201 | 201 | 307 |
| 4838 | 404 | 37 | 202 | 212 | 306 |
| 4839 | 401 | 7 | 201 | 201 | 307 |
| 4840 | 401 | 55 | 201 | 201 | 302 |
| 4841 | 406 | 77 | 212 | 212 | 301 |
| 4842 | 405 | 19 | 201 | 201 | 308 |
| 4843 | 403 | 98 | 212 | 212 | 301 |
| 4844 | 405 | 96 | 201 | 201 | 304 |
| 4845 | 405 | 74 | 201 | 201 | 303 |
| 4846 | 401 | 73 | 202 | 212 | 308 |
| 4847 | 401 | 91 | 212 | 212 | 307 |
| 4848 | 401 | 126 | 202 | 212 | 307 |
| 4849 | 405 | 86 | 212 | 212 | 302 |
| 4850 | 401 | 80 | 212 | 212 | 306 |
| 4851 | 403 | 102 | 201 | 201 | 308 |
| 4852 | 407 | 86 | 212 | 212 | 303 |
| 4853 | 401 | 58 | 212 | 212 | 306 |
| 4854 | 405 | 24 | 201 | 201 | 301 |
| 4855 | 401 | 26 | 212 | 212 | 303 |
| 4856 | 407 | 5 | 201 | 201 | 302 |
| 4857 | 402 | 76 | 202 | 212 | 306 |
| 4858 | 401 | 89 | 202 | 212 | 306 |
| 4859 | 402 | 6 | 202 | 212 | 306 |
| 4860 | 407 | 57 | 212 | 212 | 308 |
| 4861 | 401 | 96 | 201 | 201 | 308 |
| 4862 | 407 | 100 | 212 | 212 | 308 |
| 4863 | 406 | 129 | 202 | 212 | 302 |
| 4864 | 404 | 96 | 212 | 212 | 305 |
| 4865 | 401 | 20 | 201 | 201 | 305 |
| 4866 | 404 | 85 | 212 | 212 | 308 |
| 4867 | 401 | 97 | 201 | 201 | 304 |
| 4868 | 404 | 96 | 201 | 201 | 302 |
| 4869 | 401 | 85 | 212 | 212 | 307 |
| 4870 | 403 | 14 | 201 | 201 | 308 |
| 4871 | 403 | 46 | 202 | 212 | 308 |
| 4872 | 401 | 21 | 202 | 212 | 301 |
| 4873 | 404 | 96 | 212 | 212 | 307 |
| 4874 | 401 | 34 | 201 | 201 | 304 |
| 4875 | 402 | 83 | 212 | 212 | 303 |
| 4876 | 406 | 96 | 201 | 201 | 308 |
| 4877 | 402 | 32 | 212 | 212 | 301 |
| 4878 | 401 | 98 | 201 | 201 | 307 |
| 4879 | 401 | 101 | 212 | 212 | 308 |
| 4880 | 407 | 12 | 201 | 201 | 303 |
| 4881 | 405 | 85 | 212 | 212 | 301 |
| 4882 | 406 | 128 | 201 | 201 | 303 |
| 4883 | 403 | 72 | 202 | 212 | 305 |
| 4884 | 401 | 69 | 201 | 201 | 303 |
| 4885 | 401 | 15 | 212 | 212 | 302 |
| 4886 | 401 | 104 | 202 | 212 | 307 |
| 4887 | 401 | 113 | 201 | 201 | 307 |
| 4888 | 402 | 11 | 212 | 212 | 306 |
| 4889 | 402 | 108 | 212 | 212 | 302 |
| 4890 | 402 | 27 | 202 | 212 | 306 |
| 4891 | 403 | 101 | 201 | 201 | 306 |
| 4892 | 401 | 40 | 202 | 212 | 306 |
| 4893 | 401 | 96 | 201 | 201 | 301 |
| 4894 | 401 | 123 | 201 | 201 | 307 |
| 4895 | 401 | 29 | 202 | 212 | 306 |
| 4896 | 403 | 61 | 202 | 212 | 301 |
| 4897 | 406 | 122 | 201 | 201 | 308 |
| 4898 | 405 | 105 | 202 | 212 | 306 |
| 4899 | 404 | 8 | 212 | 212 | 301 |
| 4900 | 402 | 109 | 201 | 201 | 306 |
| 4901 | 405 | 101 | 201 | 201 | 301 |
| 4902 | 401 | 96 | 201 | 201 | 301 |
| 4903 | 401 | 23 | 202 | 212 | 301 |
| 4904 | 403 | 88 | 201 | 201 | 306 |
| 4905 | 401 | 22 | 201 | 201 | 308 |
| 4906 | 401 | 35 | 202 | 212 | 301 |
| 4907 | 407 | 31 | 212 | 212 | 305 |
| 4908 | 406 | 45 | 201 | 201 | 303 |
| 4909 | 406 | 109 | 201 | 201 | 303 |
| 4910 | 405 | 71 | 202 | 212 | 306 |
| 4911 | 405 | 64 | 201 | 201 | 308 |
| 4912 | 407 | 98 | 201 | 201 | 307 |
| 4913 | 402 | 70 | 201 | 201 | 302 |
| 4914 | 404 | 91 | 212 | 212 | 302 |
| 4915 | 402 | 124 | 201 | 201 | 308 |
| 4916 | 404 | 88 | 201 | 201 | 301 |
| 4917 | 401 | 1 | 212 | 212 | 308 |
| 4918 | 401 | 84 | 212 | 212 | 303 |
| 4919 | 405 | 109 | 201 | 201 | 304 |
| 4920 | 401 | 63 | 202 | 212 | 308 |
| 4921 | 406 | 103 | 201 | 201 | 301 |
| 4922 | 401 | 17 | 201 | 201 | 308 |
| 4923 | 407 | 43 | 202 | 212 | 308 |
| 4924 | 401 | 88 | 202 | 212 | 303 |
| 4925 | 401 | 88 | 212 | 212 | 307 |
| 4926 | 403 | 62 | 212 | 212 | 303 |
| 4927 | 401 | 114 | 202 | 212 | 304 |
| 4928 | 404 | 88 | 202 | 212 | 306 |
| 4929 | 401 | 65 | 212 | 212 | 306 |
| 4930 | 407 | 75 | 202 | 212 | 302 |
| 4931 | 402 | 109 | 212 | 212 | 301 |
| 4932 | 405 | 111 | 202 | 212 | 308 |
| 4933 | 407 | 119 | 212 | 212 | 302 |
| 4934 | 406 | 91 | 201 | 201 | 308 |
| 4935 | 406 | 4 | 201 | 201 | 304 |
| 4936 | 401 | 109 | 201 | 201 | 302 |
| 4937 | 403 | 88 | 202 | 212 | 306 |
| 4938 | 403 | 30 | 201 | 201 | 305 |
| 4939 | 403 | 93 | 212 | 212 | 307 |
| 4940 | 401 | 53 | 201 | 201 | 303 |
| 4941 | 402 | 68 | 212 | 212 | 304 |
| 4942 | 401 | 88 | 212 | 212 | 301 |
| 4943 | 407 | 42 | 212 | 212 | 308 |
| 4944 | 401 | 130 | 202 | 212 | 306 |
| 4945 | 401 | 85 | 202 | 212 | 301 |
| 4946 | 401 | 28 | 202 | 212 | 302 |
| 4947 | 401 | 91 | 201 | 201 | 304 |
| 4948 | 404 | 81 | 212 | 212 | 301 |
| 4949 | 401 | 82 | 202 | 212 | 302 |
| 4950 | 402 | 44 | 201 | 201 | 307 |
| 4951 | 401 | 117 | 202 | 212 | 302 |
| 4952 | 407 | 41 | 202 | 212 | 302 |

Compounds having the formula:

TABLE 29

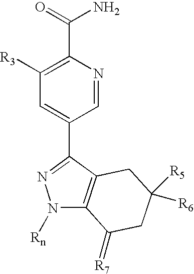

wherein R₃, Rₙ, R₅, R₆, and R₇ are defined in Table 29:

| Compound | | | | | |
|---|---|---|---|---|---|
| No. | R₃ | Rₙ | R₅ | R₆ | R₇ |
| 4953 | 85 | 204 | 212 | 212 | 308 |
| 4954 | 109 | 201 | 212 | 212 | 301 |
| 4955 | 3 | 203 | 202 | 212 | 302 |
| 4956 | 63 | 210 | 202 | 212 | 308 |
| 4957 | 8 | 204 | 212 | 212 | 301 |
| 4958 | 89 | 201 | 202 | 212 | 306 |
| 4959 | 64 | 212 | 201 | 201 | 308 |
| 4960 | 86 | 204 | 212 | 212 | 304 |
| 4961 | 36 | 212 | 201 | 201 | 301 |
| 4962 | 52 | 201 | 202 | 212 | 306 |
| 4963 | 37 | 204 | 202 | 212 | 306 |
| 4964 | 88 | 202 | 212 | 212 | 307 |
| 4965 | 4 | 201 | 201 | 201 | 304 |
| 4966 | 16 | 205 | 201 | 201 | 306 |
| 4967 | 104 | 210 | 202 | 212 | 307 |
| 4968 | 66 | 201 | 212 | 212 | 304 |
| 4969 | 68 | 204 | 212 | 212 | 304 |
| 4970 | 90 | 205 | 212 | 212 | 305 |
| 4971 | 31 | 208 | 212 | 212 | 305 |
| 4972 | 54 | 212 | 201 | 201 | 302 |
| 4973 | 28 | 212 | 202 | 212 | 302 |
| 4974 | 74 | 210 | 201 | 201 | 303 |
| 4975 | 112 | 212 | 201 | 201 | 304 |
| 4976 | 13 | 204 | 201 | 201 | 302 |
| 4977 | 121 | 202 | 202 | 212 | 308 |
| 4978 | 95 | 204 | 201 | 201 | 308 |
| 4979 | 40 | 210 | 202 | 212 | 306 |
| 4980 | 10 | 211 | 202 | 212 | 308 |
| 4981 | 98 | 212 | 201 | 201 | 307 |
| 4982 | 24 | 204 | 201 | 201 | 301 |
| 4983 | 88 | 211 | 201 | 201 | 306 |
| 4984 | 14 | 203 | 201 | 201 | 308 |
| 4985 | 101 | 203 | 201 | 201 | 301 |
| 4986 | 114 | 212 | 202 | 212 | 304 |
| 4987 | 109 | 207 | 201 | 201 | 302 |
| 4988 | 86 | 210 | 212 | 212 | 303 |
| 4989 | 105 | 212 | 202 | 212 | 306 |
| 4990 | 86 | 205 | 201 | 201 | 303 |
| 4991 | 85 | 204 | 212 | 212 | 301 |
| 4992 | 38 | 201 | 201 | 201 | 305 |
| 4993 | 48 | 212 | 202 | 212 | 303 |
| 4994 | 46 | 204 | 202 | 212 | 308 |
| 4995 | 96 | 201 | 201 | 201 | 308 |
| 4996 | 67 | 211 | 201 | 201 | 302 |
| 4997 | 19 | 202 | 201 | 201 | 308 |
| 4998 | 98 | 208 | 201 | 201 | 301 |
| 4999 | 18 | 202 | 202 | 212 | 306 |
| 5000 | 11 | 212 | 212 | 212 | 306 |
| 5001 | 123 | 201 | 201 | 201 | 307 |
| 5002 | 88 | 208 | 212 | 212 | 301 |
| 5003 | 120 | 201 | 201 | 201 | 304 |
| 5004 | 58 | 202 | 212 | 212 | 306 |
| 5005 | 49 | 202 | 201 | 201 | 307 |
| 5006 | 110 | 207 | 201 | 201 | 302 |
| 5007 | 91 | 203 | 201 | 201 | 306 |
| 5008 | 77 | 204 | 212 | 212 | 301 |
| 5009 | 21 | 212 | 202 | 212 | 301 |
| 5010 | 119 | 212 | 212 | 212 | 302 |
| 5011 | 2 | 201 | 201 | 201 | 308 |
| 5012 | 92 | 204 | 212 | 212 | 301 |
| 5013 | 32 | 211 | 212 | 212 | 301 |
| 5014 | 86 | 212 | 212 | 212 | 301 |
| 5015 | 86 | 201 | 202 | 212 | 301 |
| 5016 | 118 | 203 | 212 | 212 | 306 |
| 5017 | 97 | 210 | 201 | 201 | 304 |
| 5018 | 56 | 202 | 202 | 212 | 304 |
| 5019 | 91 | 201 | 201 | 201 | 305 |
| 5020 | 124 | 203 | 201 | 201 | 308 |
| 5021 | 85 | 212 | 212 | 212 | 307 |
| 5022 | 107 | 204 | 201 | 201 | 302 |
| 5023 | 126 | 212 | 202 | 212 | 307 |
| 5024 | 93 | 205 | 212 | 212 | 307 |
| 5025 | 88 | 201 | 202 | 212 | 303 |
| 5026 | 78 | 204 | 201 | 201 | 307 |
| 5027 | 7 | 204 | 201 | 201 | 307 |
| 5028 | 96 | 210 | 212 | 212 | 305 |
| 5029 | 27 | 212 | 202 | 212 | 306 |
| 5030 | 102 | 201 | 201 | 201 | 308 |
| 5031 | 98 | 204 | 212 | 212 | 301 |
| 5032 | 5 | 205 | 201 | 201 | 302 |
| 5033 | 81 | 210 | 212 | 212 | 301 |
| 5034 | 96 | 201 | 201 | 201 | 304 |
| 5035 | 96 | 212 | 212 | 212 | 307 |
| 5036 | 94 | 201 | 212 | 212 | 304 |
| 5037 | 44 | 202 | 201 | 201 | 307 |
| 5038 | 73 | 210 | 202 | 212 | 308 |
| 5039 | 60 | 202 | 201 | 201 | 301 |
| 5040 | 115 | 201 | 201 | 201 | 308 |
| 5041 | 128 | 212 | 201 | 201 | 303 |
| 5042 | 87 | 201 | 202 | 212 | 302 |
| 5043 | 88 | 210 | 202 | 212 | 306 |
| 5044 | 45 | 205 | 201 | 201 | 303 |
| 5045 | 91 | 212 | 201 | 201 | 304 |
| 5046 | 29 | 210 | 202 | 212 | 306 |
| 5047 | 80 | 204 | 212 | 212 | 306 |
| 5048 | 116 | 205 | 202 | 212 | 301 |
| 5049 | 109 | 212 | 201 | 201 | 305 |
| 5050 | 98 | 202 | 201 | 201 | 307 |
| 5051 | 109 | 204 | 212 | 212 | 303 |
| 5052 | 57 | 202 | 212 | 212 | 308 |
| 5053 | 99 | 203 | 202 | 212 | 307 |
| 5054 | 47 | 203 | 202 | 212 | 306 |
| 5055 | 22 | 211 | 201 | 201 | 308 |
| 5056 | 91 | 201 | 212 | 212 | 307 |
| 5057 | 35 | 202 | 202 | 212 | 301 |
| 5058 | 101 | 211 | 201 | 201 | 306 |
| 5059 | 51 | 211 | 201 | 201 | 307 |
| 5060 | 86 | 201 | 212 | 212 | 302 |
| 5061 | 72 | 204 | 202 | 212 | 305 |
| 5062 | 26 | 202 | 212 | 212 | 303 |
| 5063 | 41 | 202 | 202 | 212 | 302 |
| 5064 | 70 | 205 | 201 | 201 | 302 |
| 5065 | 88 | 210 | 201 | 201 | 301 |
| 5066 | 61 | 202 | 202 | 212 | 301 |
| 5067 | 12 | 207 | 201 | 201 | 303 |
| 5068 | 117 | 211 | 202 | 212 | 302 |
| 5069 | 20 | 201 | 201 | 201 | 305 |
| 5070 | 71 | 211 | 202 | 212 | 306 |
| 5071 | 6 | 210 | 202 | 212 | 306 |
| 5072 | 42 | 205 | 212 | 212 | 308 |
| 5073 | 96 | 211 | 201 | 201 | 301 |
| 5074 | 85 | 210 | 202 | 212 | 303 |
| 5075 | 75 | 204 | 202 | 212 | 302 |
| 5076 | 69 | 210 | 201 | 201 | 303 |
| 5077 | 106 | 205 | 212 | 212 | 307 |
| 5078 | 39 | 204 | 212 | 212 | 302 |
| 5079 | 98 | 208 | 201 | 201 | 308 |
| 5080 | 76 | 202 | 202 | 212 | 306 |
| 5081 | 59 | 201 | 201 | 201 | 306 |
| 5082 | 33 | 203 | 202 | 212 | 302 |
| 5083 | 103 | 205 | 201 | 201 | 301 |
| 5084 | 15 | 211 | 212 | 212 | 302 |
| 5085 | 109 | 207 | 201 | 201 | 304 |
| 5086 | 88 | 204 | 202 | 212 | 306 |
| 5087 | 62 | 205 | 212 | 212 | 303 |
| 5088 | 100 | 212 | 212 | 212 | 308 |
| 5089 | 53 | 204 | 201 | 201 | 303 |
| 5090 | 109 | 207 | 201 | 201 | 306 |
| 5091 | 108 | 205 | 212 | 212 | 302 |
| 5092 | 17 | 207 | 201 | 201 | 308 |
| 5093 | 125 | 201 | 212 | 212 | 308 |
| 5094 | 23 | 205 | 202 | 212 | 301 |
| 5095 | 101 | 205 | 202 | 212 | 302 |
| 5096 | 43 | 212 | 202 | 212 | 308 |
| 5097 | 129 | 211 | 202 | 212 | 302 |
| 5098 | 1 | 201 | 212 | 212 | 308 |
| 5099 | 82 | 202 | 202 | 212 | 302 |
| 5100 | 96 | 212 | 201 | 201 | 301 |
| 5101 | 34 | 205 | 201 | 201 | 304 |
| 5102 | 65 | 212 | 212 | 212 | 306 |
| 5103 | 122 | 201 | 201 | 201 | 308 |
| 5104 | 130 | 201 | 202 | 212 | 306 |
| 5105 | 91 | 210 | 201 | 201 | 308 |
| 5106 | 30 | 210 | 201 | 201 | 305 |
| 5107 | 85 | 204 | 202 | 212 | 301 |
| 5108 | 83 | 211 | 212 | 212 | 303 |
| 5109 | 98 | 205 | 201 | 201 | 302 |
| 5110 | 113 | 210 | 201 | 201 | 307 |
| 5111 | 96 | 204 | 201 | 201 | 308 |
| 5112 | 50 | 212 | 202 | 212 | 307 |
| 5113 | 84 | 203 | 212 | 212 | 303 |
| 5114 | 101 | 201 | 212 | 212 | 308 |
| 5115 | 101 | 212 | 202 | 212 | 307 |
| 5116 | 9 | 212 | 212 | 212 | 305 |
| 5117 | 79 | 201 | 212 | 212 | 301 |
| 5118 | 101 | 207 | 212 | 212 | 301 |
| 5119 | 91 | 202 | 212 | 212 | 302 |
| 5120 | 25 | 204 | 202 | 212 | 306 |
| 5121 | 55 | 204 | 201 | 201 | 302 |
| 5122 | 127 | 210 | 201 | 201 | 301 |
| 5123 | 96 | 202 | 201 | 201 | 302 |
| 5124 | 88 | 201 | 212 | 212 | 301 |
| 5125 | 111 | 204 | 202 | 212 | 308 |
| 5126 | 85 | 211 | 212 | 212 | 301 |
| 5127 | 109 | 204 | 201 | 201 | 303 |

Compounds having the formula:

TABLE 30

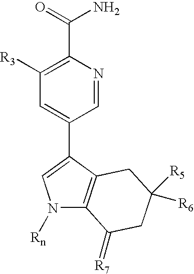

wherein R₃, Rₙ, R₅, R₆, and R₇ are defined in Table 30:

| Compound | | | | | |
|---|---|---|---|---|---|
| No. | R₃ | Rₙ | R₅ | R₆ | R₇ |
| 5128 | 73 | 210 | 202 | 212 | 308 |
| 5129 | 96 | 212 | 201 | 201 | 301 |
| 5130 | 19 | 202 | 201 | 201 | 308 |
| 5131 | 32 | 211 | 212 | 212 | 301 |
| 5132 | 29 | 210 | 202 | 212 | 306 |
| 5133 | 86 | 201 | 212 | 212 | 302 |
| 5134 | 47 | 203 | 202 | 212 | 306 |
| 5135 | 111 | 204 | 202 | 212 | 308 |
| 5136 | 96 | 202 | 201 | 201 | 302 |
| 5137 | 85 | 212 | 212 | 212 | 307 |
| 5138 | 1 | 201 | 212 | 212 | 308 |
| 5139 | 96 | 204 | 201 | 201 | 308 |
| 5140 | 49 | 202 | 201 | 201 | 307 |
| 5141 | 109 | 204 | 201 | 201 | 303 |
| 5142 | 63 | 210 | 202 | 212 | 308 |
| 5143 | 126 | 212 | 202 | 212 | 307 |
| 5144 | 83 | 211 | 212 | 212 | 303 |
| 5145 | 7 | 204 | 201 | 201 | 307 |
| 5146 | 109 | 212 | 201 | 201 | 305 |
| 5147 | 68 | 204 | 212 | 212 | 304 |
| 5148 | 107 | 204 | 201 | 201 | 302 |
| 5149 | 27 | 212 | 202 | 212 | 306 |
| 5150 | 53 | 204 | 201 | 201 | 303 |
| 5151 | 130 | 201 | 202 | 212 | 306 |
| 5152 | 91 | 203 | 201 | 201 | 306 |
| 5153 | 101 | 201 | 212 | 212 | 308 |
| 5154 | 54 | 212 | 201 | 201 | 302 |
| 5155 | 86 | 210 | 212 | 212 | 303 |
| 5156 | 75 | 204 | 202 | 212 | 302 |
| 5157 | 2 | 201 | 201 | 201 | 308 |
| 5158 | 3 | 203 | 202 | 212 | 302 |
| 5159 | 85 | 211 | 212 | 212 | 301 |
| 5160 | 20 | 201 | 201 | 201 | 305 |
| 5161 | 85 | 204 | 202 | 212 | 301 |
| 5162 | 42 | 205 | 212 | 212 | 308 |
| 5163 | 88 | 211 | 201 | 201 | 306 |
| 5164 | 96 | 201 | 201 | 201 | 304 |
| 5165 | 101 | 203 | 201 | 201 | 301 |
| 5166 | 119 | 212 | 212 | 212 | 302 |
| 5167 | 88 | 202 | 212 | 212 | 307 |
| 5168 | 96 | 212 | 212 | 212 | 307 |
| 5169 | 44 | 202 | 201 | 201 | 307 |
| 5170 | 81 | 210 | 212 | 212 | 301 |
| 5171 | 10 | 211 | 202 | 212 | 308 |
| 5172 | 128 | 212 | 201 | 201 | 303 |
| 5173 | 58 | 202 | 212 | 212 | 306 |
| 5174 | 104 | 210 | 202 | 212 | 307 |
| 5175 | 90 | 205 | 212 | 212 | 305 |
| 5176 | 77 | 204 | 212 | 212 | 301 |
| 5177 | 59 | 201 | 201 | 201 | 306 |
| 5178 | 82 | 202 | 202 | 212 | 302 |
| 5179 | 66 | 201 | 212 | 212 | 304 |
| 5180 | 88 | 204 | 202 | 212 | 306 |
| 5181 | 101 | 212 | 202 | 212 | 307 |
| 5182 | 84 | 203 | 212 | 212 | 303 |
| 5183 | 98 | 208 | 201 | 201 | 301 |
| 5184 | 51 | 211 | 201 | 201 | 307 |
| 5185 | 8 | 204 | 212 | 212 | 301 |
| 5186 | 99 | 203 | 202 | 212 | 307 |
| 5187 | 101 | 207 | 212 | 212 | 301 |
| 5188 | 110 | 207 | 201 | 201 | 302 |
| 5189 | 98 | 212 | 201 | 201 | 307 |
| 5190 | 121 | 202 | 202 | 212 | 308 |
| 5191 | 26 | 202 | 212 | 212 | 303 |
| 5192 | 40 | 210 | 202 | 212 | 306 |
| 5193 | 64 | 212 | 201 | 201 | 308 |
| 5194 | 67 | 211 | 201 | 201 | 302 |
| 5195 | 61 | 202 | 202 | 212 | 301 |
| 5196 | 91 | 212 | 201 | 201 | 304 |
| 5197 | 113 | 210 | 201 | 201 | 307 |
| 5198 | 85 | 210 | 202 | 212 | 303 |
| 5199 | 96 | 211 | 201 | 201 | 301 |
| 5200 | 62 | 205 | 212 | 212 | 303 |
| 5201 | 34 | 205 | 201 | 201 | 304 |
| 5202 | 52 | 201 | 202 | 212 | 306 |
| 5203 | 55 | 204 | 201 | 201 | 302 |
| 5204 | 57 | 202 | 212 | 212 | 308 |
| 5205 | 117 | 211 | 202 | 212 | 302 |
| 5206 | 12 | 207 | 201 | 201 | 303 |
| 5207 | 129 | 211 | 202 | 212 | 302 |
| 5208 | 86 | 204 | 212 | 212 | 304 |
| 5209 | 93 | 205 | 212 | 212 | 307 |
| 5210 | 79 | 201 | 212 | 212 | 301 |
| 5211 | 25 | 204 | 202 | 212 | 306 |
| 5212 | 41 | 202 | 202 | 212 | 302 |
| 5213 | 89 | 201 | 202 | 212 | 306 |
| 5214 | 86 | 212 | 212 | 212 | 301 |
| 5215 | 6 | 210 | 202 | 212 | 306 |
| 5216 | 24 | 204 | 201 | 201 | 301 |
| 5217 | 45 | 205 | 201 | 201 | 303 |
| 5218 | 86 | 201 | 202 | 212 | 301 |
| 5219 | 4 | 201 | 201 | 201 | 304 |
| 5220 | 88 | 201 | 212 | 212 | 301 |
| 5221 | 118 | 203 | 212 | 212 | 306 |
| 5222 | 91 | 210 | 201 | 201 | 308 |
| 5223 | 37 | 204 | 202 | 212 | 306 |
| 5224 | 50 | 212 | 202 | 212 | 307 |
| 5225 | 5 | 205 | 201 | 201 | 302 |
| 5226 | 60 | 202 | 201 | 201 | 301 |
| 5227 | 125 | 201 | 212 | 212 | 308 |
| 5228 | 30 | 210 | 201 | 201 | 305 |
| 5229 | 103 | 205 | 201 | 201 | 301 |
| 5230 | 46 | 204 | 202 | 212 | 308 |
| 5231 | 13 | 204 | 201 | 201 | 302 |
| 5232 | 123 | 201 | 201 | 201 | 307 |
| 5233 | 109 | 207 | 201 | 201 | 304 |
| 5234 | 17 | 207 | 201 | 201 | 308 |
| 5235 | 98 | 205 | 201 | 201 | 302 |
| 5236 | 109 | 207 | 201 | 201 | 302 |
| 5237 | 11 | 212 | 212 | 212 | 306 |
| 5238 | 88 | 208 | 212 | 212 | 301 |
| 5239 | 28 | 212 | 202 | 212 | 302 |
| 5240 | 88 | 210 | 201 | 201 | 301 |
| 5241 | 122 | 201 | 201 | 201 | 308 |
| 5242 | 85 | 204 | 212 | 212 | 301 |
| 5243 | 69 | 210 | 201 | 201 | 303 |
| 5244 | 112 | 212 | 201 | 201 | 304 |
| 5245 | 33 | 203 | 202 | 212 | 302 |
| 5246 | 23 | 205 | 202 | 212 | 301 |
| 5247 | 70 | 205 | 201 | 201 | 302 |
| 5248 | 35 | 202 | 202 | 212 | 301 |
| 5249 | 109 | 204 | 212 | 212 | 303 |
| 5250 | 105 | 212 | 202 | 212 | 306 |
| 5251 | 127 | 210 | 201 | 201 | 301 |
| 5252 | 16 | 205 | 201 | 201 | 306 |
| 5253 | 48 | 212 | 202 | 212 | 303 |
| 5254 | 109 | 201 | 212 | 212 | 301 |
| 5255 | 22 | 211 | 201 | 201 | 308 |
| 5256 | 74 | 210 | 201 | 201 | 303 |
| 5257 | 88 | 210 | 202 | 212 | 306 |
| 5258 | 100 | 212 | 212 | 212 | 308 |
| 5259 | 31 | 208 | 212 | 212 | 305 |
| 5260 | 80 | 204 | 212 | 212 | 306 |
| 5261 | 91 | 201 | 212 | 212 | 307 |
| 5262 | 88 | 201 | 202 | 212 | 303 |
| 5263 | 72 | 204 | 202 | 212 | 305 |
| 5264 | 120 | 201 | 201 | 201 | 304 |
| 5265 | 18 | 202 | 202 | 212 | 306 |
| 5266 | 106 | 205 | 212 | 212 | 307 |
| 5267 | 97 | 210 | 201 | 201 | 304 |
| 5268 | 94 | 201 | 212 | 212 | 304 |
| 5269 | 108 | 205 | 212 | 212 | 302 |
| 5270 | 85 | 204 | 212 | 212 | 308 |
| 5271 | 21 | 212 | 202 | 212 | 301 |
| 5272 | 39 | 204 | 212 | 212 | 302 |
| 5273 | 91 | 202 | 212 | 212 | 302 |
| 5274 | 96 | 210 | 212 | 212 | 305 |
| 5275 | 101 | 205 | 202 | 212 | 302 |
| 5276 | 76 | 202 | 202 | 212 | 306 |
| 5277 | 92 | 204 | 212 | 212 | 301 |
| 5278 | 102 | 201 | 201 | 201 | 308 |
| 5279 | 71 | 211 | 202 | 212 | 306 |
| 5280 | 91 | 201 | 201 | 201 | 305 |
| 5281 | 56 | 202 | 202 | 212 | 304 |
| 5282 | 15 | 211 | 212 | 212 | 302 |
| 5283 | 9 | 212 | 212 | 212 | 305 |
| 5284 | 96 | 201 | 201 | 201 | 308 |
| 5285 | 78 | 204 | 201 | 201 | 307 |
| 5286 | 114 | 212 | 202 | 212 | 304 |
| 5287 | 124 | 203 | 201 | 201 | 308 |
| 5288 | 87 | 201 | 202 | 212 | 302 |
| 5289 | 98 | 204 | 212 | 212 | 301 |
| 5290 | 109 | 207 | 201 | 201 | 306 |
| 5291 | 38 | 201 | 201 | 201 | 305 |
| 5292 | 115 | 201 | 201 | 201 | 308 |
| 5293 | 86 | 205 | 201 | 201 | 303 |
| 5294 | 14 | 203 | 201 | 201 | 308 |
| 5295 | 101 | 211 | 201 | 201 | 306 |
| 5296 | 116 | 205 | 202 | 212 | 301 |
| 5297 | 36 | 212 | 201 | 201 | 301 |
| 5298 | 43 | 212 | 202 | 212 | 308 |
| 5299 | 65 | 212 | 212 | 212 | 306 |
| 5300 | 98 | 202 | 201 | 201 | 307 |
| 5301 | 98 | 208 | 201 | 201 | 308 |

Compounds having the formula:

TABLE 31

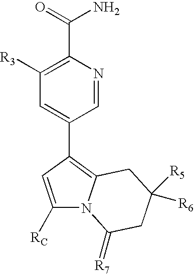

wherein R₃, R_{C}, R₅, R₆, and R₇ are defined in Table 31:

| Compound | | | | | |
|---|---|---|---|---|---|
| No. | R₃ | R_{C} | R₅ | R₆ | R₇ |
| 5302 | 65 | 212 | 212 | 212 | 306 |
| 5303 | 71 | 211 | 202 | 212 | 306 |
| 5304 | 88 | 210 | 201 | 201 | 301 |
| 5305 | 33 | 203 | 202 | 212 | 302 |
| 5306 | 127 | 210 | 201 | 201 | 301 |
| 5307 | 64 | 212 | 201 | 201 | 308 |
| 5308 | 123 | 201 | 201 | 201 | 307 |
| 5309 | 88 | 206 | 202 | 212 | 303 |
| 5310 | 27 | 212 | 202 | 212 | 306 |
| 5311 | 112 | 212 | 201 | 201 | 304 |
| 5312 | 75 | 204 | 202 | 212 | 302 |
| 5313 | 96 | 209 | 201 | 201 | 302 |
| 5314 | 86 | 205 | 201 | 201 | 303 |
| 5315 | 101 | 201 | 212 | 212 | 308 |
| 5316 | 48 | 212 | 202 | 212 | 303 |
| 5317 | 3 | 203 | 202 | 212 | 302 |
| 5318 | 88 | 206 | 212 | 212 | 301 |
| 5319 | 50 | 212 | 202 | 212 | 307 |
| 5320 | 119 | 212 | 212 | 212 | 302 |
| 5321 | 88 | 209 | 212 | 212 | 307 |
| 5322 | 60 | 202 | 201 | 201 | 301 |
| 5323 | 78 | 204 | 201 | 201 | 307 |
| 5324 | 101 | 205 | 202 | 212 | 302 |
| 5325 | 7 | 204 | 201 | 201 | 307 |
| 5326 | 46 | 204 | 202 | 212 | 308 |
| 5327 | 109 | 207 | 201 | 201 | 304 |
| 5328 | 1 | 201 | 212 | 212 | 308 |
| 5329 | 66 | 201 | 212 | 212 | 304 |
| 5330 | 9 | 212 | 212 | 212 | 305 |
| 5331 | 39 | 204 | 212 | 212 | 302 |
| 5332 | 74 | 210 | 201 | 201 | 303 |
| 5333 | 98 | 208 | 201 | 201 | 301 |
| 5334 | 26 | 209 | 212 | 212 | 303 |
| 5335 | 129 | 211 | 202 | 212 | 302 |
| 5336 | 96 | 212 | 201 | 201 | 301 |
| 5337 | 40 | 210 | 202 | 212 | 306 |
| 5338 | 108 | 205 | 212 | 212 | 302 |
| 5339 | 109 | 201 | 212 | 212 | 301 |
| 5340 | 98 | 212 | 201 | 201 | 307 |
| 5341 | 85 | 204 | 212 | 212 | 301 |
| 5342 | 86 | 201 | 202 | 212 | 301 |
| 5343 | 11 | 212 | 212 | 212 | 306 |
| 5344 | 89 | 206 | 202 | 212 | 306 |
| 5345 | 51 | 211 | 201 | 201 | 307 |
| 5346 | 85 | 212 | 212 | 212 | 307 |
| 5347 | 49 | 209 | 201 | 201 | 307 |
| 5348 | 91 | 203 | 201 | 201 | 306 |
| 5349 | 91 | 209 | 212 | 212 | 302 |
| 5350 | 83 | 211 | 212 | 212 | 303 |
| 5351 | 68 | 204 | 212 | 212 | 304 |
| 5352 | 88 | 210 | 202 | 212 | 306 |
| 5353 | 111 | 204 | 202 | 212 | 308 |
| 5354 | 98 | 204 | 212 | 212 | 301 |
| 5355 | 104 | 210 | 202 | 212 | 307 |
| 5356 | 6 | 210 | 202 | 212 | 306 |
| 5357 | 22 | 211 | 201 | 201 | 308 |
| 5358 | 110 | 207 | 201 | 201 | 302 |
| 5359 | 70 | 205 | 201 | 201 | 302 |
| 5360 | 44 | 202 | 201 | 201 | 307 |
| 5361 | 99 | 203 | 202 | 212 | 307 |
| 5362 | 23 | 205 | 202 | 212 | 301 |
| 5363 | 15 | 211 | 212 | 212 | 302 |
| 5364 | 98 | 205 | 201 | 201 | 302 |
| 5365 | 121 | 209 | 202 | 212 | 308 |
| 5366 | 82 | 209 | 202 | 212 | 302 |
| 5367 | 126 | 212 | 202 | 212 | 307 |
| 5368 | 57 | 202 | 212 | 212 | 308 |
| 5369 | 93 | 205 | 212 | 212 | 307 |
| 5370 | 88 | 211 | 201 | 201 | 306 |
| 5371 | 95 | 204 | 201 | 201 | 308 |
| 5372 | 30 | 210 | 201 | 201 | 305 |
| 5373 | 88 | 208 | 212 | 212 | 301 |
| 5374 | 85 | 210 | 202 | 212 | 303 |
| 5375 | 91 | 201 | 201 | 201 | 305 |
| 5376 | 122 | 201 | 201 | 201 | 308 |
| 5377 | 35 | 209 | 202 | 212 | 301 |
| 5378 | 47 | 203 | 202 | 212 | 306 |
| 5379 | 107 | 204 | 201 | 201 | 302 |
| 5380 | 43 | 212 | 202 | 212 | 308 |
| 5381 | 61 | 202 | 202 | 212 | 301 |
| 5382 | 97 | 210 | 201 | 201 | 304 |
| 5383 | 41 | 202 | 202 | 212 | 302 |
| 5384 | 86 | 204 | 212 | 212 | 304 |
| 5385 | 88 | 204 | 202 | 212 | 306 |
| 5386 | 109 | 204 | 212 | 212 | 303 |
| 5387 | 91 | 206 | 212 | 212 | 307 |
| 5388 | 12 | 207 | 201 | 201 | 303 |
| 5389 | 36 | 212 | 201 | 201 | 301 |
| 5390 | 109 | 204 | 201 | 201 | 303 |
| 5391 | 16 | 205 | 201 | 201 | 306 |
| 5392 | 10 | 211 | 202 | 212 | 308 |
| 5393 | 37 | 204 | 202 | 212 | 306 |
| 5394 | 92 | 204 | 212 | 212 | 301 |
| 5395 | 117 | 211 | 202 | 212 | 302 |
| 5396 | 100 | 212 | 212 | 212 | 308 |
| 5397 | 101 | 207 | 212 | 212 | 301 |
| 5398 | 101 | 212 | 202 | 212 | 307 |
| 5399 | 98 | 208 | 201 | 201 | 308 |
| 5400 | 113 | 210 | 201 | 201 | 307 |
| 5401 | 2 | 201 | 201 | 201 | 308 |
| 5402 | 31 | 208 | 212 | 212 | 305 |
| 5403 | 32 | 211 | 212 | 212 | 301 |
| 5404 | 55 | 204 | 201 | 201 | 302 |
| 5405 | 96 | 211 | 201 | 201 | 301 |
| 5406 | 38 | 206 | 201 | 201 | 305 |
| 5407 | 109 | 207 | 201 | 201 | 302 |
| 5408 | 116 | 205 | 202 | 212 | 301 |
| 5409 | 85 | 211 | 212 | 212 | 301 |
| 5410 | 115 | 206 | 201 | 201 | 308 |
| 5411 | 52 | 201 | 202 | 212 | 306 |
| 5412 | 25 | 204 | 202 | 212 | 306 |
| 5413 | 86 | 210 | 212 | 212 | 303 |
| 5414 | 42 | 205 | 212 | 212 | 308 |
| 5415 | 102 | 201 | 201 | 201 | 308 |
| 5416 | 101 | 211 | 201 | 201 | 306 |
| 5417 | 109 | 212 | 201 | 201 | 305 |
| 5418 | 63 | 210 | 202 | 212 | 308 |
| 5419 | 81 | 210 | 212 | 212 | 301 |
| 5420 | 128 | 212 | 201 | 201 | 303 |
| 5421 | 79 | 206 | 212 | 212 | 301 |
| 5422 | 85 | 204 | 212 | 212 | 308 |
| 5423 | 85 | 204 | 202 | 212 | 301 |
| 5424 | 96 | 201 | 201 | 201 | 304 |
| 5425 | 103 | 205 | 201 | 201 | 301 |
| 5426 | 114 | 212 | 202 | 212 | 304 |
| 5427 | 56 | 209 | 202 | 212 | 304 |
| 5428 | 77 | 204 | 212 | 212 | 301 |
| 5429 | 24 | 204 | 201 | 201 | 301 |
| 5430 | 120 | 201 | 201 | 201 | 304 |
| 5431 | 109 | 207 | 201 | 201 | 306 |
| 5432 | 53 | 204 | 201 | 201 | 303 |
| 5433 | 84 | 203 | 212 | 212 | 303 |
| 5434 | 87 | 206 | 202 | 212 | 302 |
| 5435 | 96 | 212 | 212 | 212 | 307 |
| 5436 | 17 | 207 | 201 | 201 | 308 |
| 5437 | 130 | 206 | 202 | 212 | 306 |
| 5438 | 91 | 212 | 201 | 201 | 304 |
| 5439 | 62 | 205 | 212 | 212 | 303 |
| 5440 | 72 | 204 | 202 | 212 | 305 |
| 5441 | 67 | 211 | 201 | 201 | 302 |
| 5442 | 90 | 205 | 212 | 212 | 305 |
| 5443 | 80 | 204 | 212 | 212 | 306 |
| 5444 | 59 | 206 | 201 | 201 | 306 |
| 5445 | 58 | 202 | 212 | 212 | 306 |
| 5446 | 45 | 205 | 201 | 201 | 303 |
| 5447 | 73 | 210 | 202 | 212 | 308 |
| 5448 | 118 | 203 | 212 | 212 | 306 |
| 5449 | 8 | 204 | 212 | 212 | 301 |
| 5450 | 125 | 201 | 212 | 212 | 308 |
| 5451 | 96 | 201 | 201 | 201 | 308 |
| 5452 | 34 | 205 | 201 | 201 | 304 |
| 5453 | 106 | 205 | 212 | 212 | 307 |
| 5454 | 18 | 202 | 202 | 212 | 306 |
| 5455 | 13 | 204 | 201 | 201 | 302 |
| 5456 | 98 | 209 | 201 | 201 | 307 |
| 5457 | 54 | 212 | 201 | 201 | 302 |
| 5458 | 91 | 210 | 201 | 201 | 308 |
| 5459 | 28 | 212 | 202 | 212 | 302 |
| 5460 | 86 | 212 | 212 | 212 | 301 |
| 5461 | 96 | 210 | 212 | 212 | 305 |
| 5462 | 19 | 202 | 201 | 201 | 308 |
| 5463 | 105 | 212 | 202 | 212 | 306 |
| 5464 | 96 | 204 | 201 | 201 | 308 |
| 5465 | 124 | 203 | 201 | 201 | 308 |
| 5466 | 4 | 201 | 201 | 201 | 304 |
| 5467 | 76 | 202 | 202 | 212 | 306 |
| 5468 | 21 | 212 | 202 | 212 | 301 |
| 5469 | 5 | 205 | 201 | 201 | 302 |
| 5470 | 101 | 203 | 201 | 201 | 301 |
| 5471 | 20 | 201 | 201 | 201 | 305 |
| 5472 | 69 | 210 | 201 | 201 | 303 |
| 5473 | 29 | 210 | 202 | 212 | 306 |
| 5474 | 94 | 201 | 212 | 212 | 304 |
| 5475 | 14 | 203 | 201 | 201 | 308 |
| 5476 | 86 | 201 | 212 | 212 | 302 |

Compounds having the formula:

TABLE 32

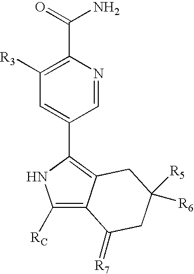

wherein R₃, R_{C}, R₅, R₆, and R₇ are defined in Table 32:

| Compound | | | | | |
|---|---|---|---|---|---|
| No. | R₃ | R_{C} | R₅ | R₆ | R₇ |
| 5477 | 32 | 211 | 212 | 212 | 301 |
| 5478 | 86 | 210 | 212 | 212 | 303 |
| 5479 | 86 | 204 | 212 | 212 | 304 |
| 5480 | 96 | 209 | 201 | 201 | 302 |
| 5481 | 27 | 212 | 202 | 212 | 306 |
| 5482 | 109 | 207 | 201 | 201 | 306 |
| 5483 | 67 | 211 | 201 | 201 | 302 |
| 5484 | 39 | 204 | 212 | 212 | 302 |
| 5485 | 96 | 201 | 201 | 201 | 304 |
| 5486 | 37 | 204 | 202 | 212 | 306 |
| 5487 | 54 | 212 | 201 | 201 | 302 |
| 5488 | 55 | 204 | 201 | 201 | 302 |
| 5489 | 85 | 204 | 202 | 212 | 301 |
| 5490 | 106 | 205 | 212 | 212 | 307 |
| 5491 | 96 | 204 | 201 | 201 | 308 |
| 5492 | 88 | 210 | 202 | 212 | 306 |
| 5493 | 70 | 205 | 201 | 201 | 302 |
| 5494 | 109 | 207 | 201 | 201 | 304 |
| 5495 | 6 | 210 | 202 | 212 | 306 |
| 5496 | 58 | 202 | 212 | 212 | 306 |
| 5497 | 105 | 212 | 202 | 212 | 306 |
| 5498 | 25 | 204 | 202 | 212 | 306 |
| 5499 | 101 | 201 | 212 | 212 | 308 |
| 5500 | 74 | 210 | 201 | 201 | 303 |
| 5501 | 116 | 205 | 202 | 212 | 301 |
| 5502 | 119 | 212 | 212 | 212 | 302 |
| 5503 | 104 | 210 | 202 | 212 | 307 |
| 5504 | 21 | 212 | 202 | 212 | 301 |
| 5505 | 93 | 205 | 212 | 212 | 307 |
| 5506 | 48 | 212 | 202 | 212 | 303 |
| 5507 | 96 | 212 | 201 | 201 | 301 |
| 5508 | 69 | 210 | 201 | 201 | 303 |
| 5509 | 117 | 211 | 202 | 212 | 302 |
| 5510 | 110 | 207 | 201 | 201 | 302 |
| 5511 | 78 | 204 | 201 | 201 | 307 |
| 5512 | 34 | 205 | 201 | 201 | 304 |
| 5513 | 53 | 204 | 201 | 201 | 303 |
| 5514 | 19 | 202 | 201 | 201 | 308 |
| 5515 | 2 | 201 | 201 | 201 | 308 |
| 5516 | 96 | 212 | 212 | 212 | 307 |
| 5517 | 107 | 204 | 201 | 201 | 302 |
| 5518 | 15 | 211 | 212 | 212 | 302 |
| 5519 | 85 | 204 | 212 | 212 | 301 |
| 5520 | 65 | 212 | 212 | 212 | 306 |
| 5521 | 101 | 212 | 202 | 212 | 307 |
| 5522 | 98 | 205 | 201 | 201 | 302 |
| 5523 | 88 | 208 | 212 | 212 | 301 |
| 5524 | 66 | 201 | 212 | 212 | 304 |
| 5525 | 97 | 210 | 201 | 201 | 304 |
| 5526 | 101 | 205 | 202 | 212 | 302 |
| 5527 | 79 | 206 | 212 | 212 | 301 |
| 5528 | 36 | 212 | 201 | 201 | 301 |
| 5529 | 42 | 205 | 212 | 212 | 308 |
| 5530 | 61 | 202 | 202 | 212 | 301 |
| 5531 | 109 | 201 | 212 | 212 | 301 |
| 5532 | 43 | 212 | 202 | 212 | 308 |
| 5533 | 118 | 203 | 212 | 212 | 306 |
| 5534 | 101 | 207 | 212 | 212 | 301 |
| 5535 | 85 | 211 | 212 | 212 | 301 |
| 5536 | 17 | 207 | 201 | 201 | 308 |
| 5537 | 35 | 209 | 202 | 212 | 301 |
| 5538 | 11 | 212 | 212 | 212 | 306 |
| 5539 | 108 | 205 | 212 | 212 | 302 |
| 5540 | 88 | 211 | 201 | 201 | 306 |
| 5541 | 98 | 209 | 201 | 201 | 307 |
| 5542 | 122 | 201 | 201 | 201 | 308 |
| 5543 | 125 | 201 | 212 | 212 | 308 |
| 5544 | 1 | 201 | 212 | 212 | 308 |
| 5545 | 51 | 211 | 201 | 201 | 307 |
| 5546 | 91 | 203 | 201 | 201 | 306 |
| 5547 | 90 | 205 | 212 | 212 | 305 |
| 5548 | 7 | 204 | 201 | 201 | 307 |
| 5549 | 86 | 205 | 201 | 201 | 303 |
| 5550 | 96 | 211 | 201 | 201 | 301 |
| 5551 | 41 | 202 | 202 | 212 | 302 |
| 5552 | 28 | 212 | 202 | 212 | 302 |
| 5553 | 86 | 201 | 212 | 212 | 302 |
| 5554 | 10 | 211 | 202 | 212 | 308 |
| 5555 | 16 | 205 | 201 | 201 | 306 |
| 5556 | 77 | 204 | 212 | 212 | 301 |
| 5557 | 52 | 201 | 202 | 212 | 306 |
| 5558 | 109 | 204 | 201 | 201 | 303 |
| 5559 | 94 | 201 | 212 | 212 | 304 |
| 5560 | 113 | 210 | 201 | 201 | 307 |
| 5561 | 75 | 204 | 202 | 212 | 302 |
| 5562 | 112 | 212 | 201 | 201 | 304 |
| 5563 | 84 | 203 | 212 | 212 | 303 |
| 5564 | 85 | 204 | 212 | 212 | 308 |
| 5565 | 56 | 209 | 202 | 212 | 304 |
| 5566 | 109 | 207 | 201 | 201 | 302 |
| 5567 | 124 | 203 | 201 | 201 | 308 |
| 5568 | 126 | 212 | 202 | 212 | 307 |
| 5569 | 8 | 204 | 212 | 212 | 301 |
| 5570 | 101 | 211 | 201 | 201 | 306 |
| 5571 | 88 | 210 | 201 | 201 | 301 |
| 5572 | 85 | 212 | 212 | 212 | 307 |
| 5573 | 64 | 212 | 201 | 201 | 308 |
| 5574 | 98 | 208 | 201 | 201 | 308 |
| 5575 | 38 | 206 | 201 | 201 | 305 |
| 5576 | 109 | 204 | 212 | 212 | 303 |
| 5577 | 80 | 204 | 212 | 212 | 306 |
| 5578 | 18 | 202 | 202 | 212 | 306 |
| 5579 | 60 | 202 | 201 | 201 | 301 |
| 5580 | 24 | 204 | 201 | 201 | 301 |
| 5581 | 76 | 202 | 202 | 212 | 306 |
| 5582 | 46 | 204 | 202 | 212 | 308 |
| 5583 | 33 | 203 | 202 | 212 | 302 |
| 5584 | 31 | 208 | 212 | 212 | 305 |
| 5585 | 87 | 206 | 202 | 212 | 302 |
| 5586 | 71 | 211 | 202 | 212 | 306 |
| 5587 | 115 | 206 | 201 | 201 | 308 |
| 5588 | 96 | 201 | 201 | 201 | 308 |
| 5589 | 98 | 204 | 212 | 212 | 301 |
| 5590 | 123 | 201 | 201 | 201 | 307 |
| 5591 | 101 | 203 | 201 | 201 | 301 |
| 5592 | 9 | 212 | 212 | 212 | 305 |
| 5593 | 91 | 201 | 201 | 201 | 305 |
| 5594 | 111 | 204 | 202 | 212 | 308 |
| 5595 | 72 | 204 | 202 | 212 | 305 |
| 5596 | 44 | 202 | 201 | 201 | 307 |
| 5597 | 128 | 212 | 201 | 201 | 303 |
| 5598 | 127 | 210 | 201 | 201 | 301 |
| 5599 | 91 | 206 | 212 | 212 | 307 |
| 5600 | 49 | 209 | 201 | 201 | 307 |
| 5601 | 30 | 210 | 201 | 201 | 305 |
| 5602 | 82 | 209 | 202 | 212 | 302 |
| 5603 | 130 | 206 | 202 | 212 | 306 |
| 5604 | 91 | 209 | 212 | 212 | 302 |
| 5605 | 63 | 210 | 202 | 212 | 308 |
| 5606 | 40 | 210 | 202 | 212 | 306 |
| 5607 | 29 | 210 | 202 | 212 | 306 |
| 5608 | 85 | 210 | 202 | 212 | 303 |
| 5609 | 73 | 210 | 202 | 212 | 308 |
| 5610 | 4 | 201 | 201 | 201 | 304 |
| 5611 | 86 | 212 | 212 | 212 | 301 |
| 5612 | 81 | 210 | 212 | 212 | 301 |
| 5613 | 98 | 212 | 201 | 201 | 307 |
| 5614 | 88 | 209 | 212 | 212 | 307 |
| 5615 | 57 | 202 | 212 | 212 | 308 |
| 5616 | 86 | 201 | 202 | 212 | 301 |
| 5617 | 20 | 201 | 201 | 201 | 305 |
| 5618 | 14 | 203 | 201 | 201 | 308 |
| 5619 | 68 | 204 | 212 | 212 | 304 |
| 5620 | 88 | 206 | 202 | 212 | 303 |
| 5621 | 99 | 203 | 202 | 212 | 307 |
| 5622 | 114 | 212 | 202 | 212 | 304 |
| 5623 | 47 | 203 | 202 | 212 | 306 |
| 5624 | 12 | 207 | 201 | 201 | 303 |
| 5625 | 62 | 205 | 212 | 212 | 303 |
| 5626 | 109 | 212 | 201 | 201 | 305 |
| 5627 | 89 | 206 | 202 | 212 | 306 |
| 5628 | 92 | 204 | 212 | 212 | 301 |
| 5629 | 45 | 205 | 201 | 201 | 303 |
| 5630 | 22 | 211 | 201 | 201 | 308 |
| 5631 | 59 | 206 | 201 | 201 | 306 |
| 5632 | 96 | 210 | 212 | 212 | 305 |
| 5633 | 120 | 201 | 201 | 201 | 304 |
| 5634 | 50 | 212 | 202 | 212 | 307 |
| 5635 | 103 | 205 | 201 | 201 | 301 |
| 5636 | 83 | 211 | 212 | 212 | 303 |
| 5637 | 13 | 204 | 201 | 201 | 302 |
| 5638 | 91 | 210 | 201 | 201 | 308 |
| 5639 | 91 | 212 | 201 | 201 | 304 |
| 5640 | 129 | 211 | 202 | 212 | 302 |
| 5641 | 5 | 205 | 201 | 201 | 302 |
| 5642 | 3 | 203 | 202 | 212 | 302 |
| 5643 | 102 | 201 | 201 | 201 | 308 |
| 5644 | 26 | 209 | 212 | 212 | 303 |
| 5645 | 88 | 206 | 212 | 212 | 301 |
| 5646 | 23 | 205 | 202 | 212 | 301 |
| 5647 | 88 | 204 | 202 | 212 | 306 |
| 5648 | 121 | 209 | 202 | 212 | 308 |
| 5649 | 98 | 208 | 201 | 201 | 301 |
| 5650 | 100 | 212 | 212 | 212 | 308 |

Compounds having the formula:

TABLE 33

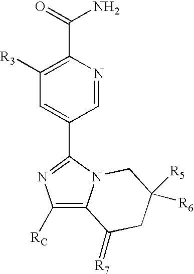

wherein R₃, R_{C}, R₅, R₆, and R₇ are defined in Table 33:

| Compound | | | | | |
|---|---|---|---|---|---|
| No. | R₃ | R_{C} | R₅ | R₆ | R₇ |
| 5651 | 88 | 209 | 212 | 212 | 307 |
| 5652 | 55 | 204 | 201 | 201 | 302 |
| 5653 | 88 | 210 | 201 | 201 | 301 |
| 5654 | 54 | 212 | 201 | 201 | 302 |
| 5655 | 17 | 207 | 201 | 201 | 308 |
| 5656 | 109 | 204 | 201 | 201 | 303 |
| 5657 | 61 | 202 | 202 | 212 | 301 |
| 5658 | 88 | 208 | 212 | 212 | 301 |
| 5659 | 130 | 206 | 202 | 212 | 306 |
| 5660 | 128 | 212 | 201 | 201 | 303 |
| 5661 | 101 | 203 | 201 | 201 | 301 |
| 5662 | 105 | 212 | 202 | 212 | 306 |
| 5663 | 52 | 201 | 202 | 212 | 306 |
| 5664 | 46 | 204 | 202 | 212 | 308 |
| 5665 | 15 | 211 | 212 | 212 | 302 |
| 5666 | 109 | 207 | 201 | 201 | 302 |
| 5667 | 63 | 210 | 202 | 212 | 308 |
| 5668 | 82 | 209 | 202 | 212 | 302 |
| 5669 | 85 | 210 | 202 | 212 | 303 |
| 5670 | 80 | 204 | 212 | 212 | 306 |
| 5671 | 124 | 203 | 201 | 201 | 308 |
| 5672 | 29 | 210 | 202 | 212 | 306 |
| 5673 | 7 | 204 | 201 | 201 | 307 |
| 5674 | 88 | 211 | 201 | 201 | 306 |
| 5675 | 27 | 212 | 202 | 212 | 306 |
| 5676 | 73 | 210 | 202 | 212 | 308 |
| 5677 | 86 | 205 | 201 | 201 | 303 |
| 5678 | 106 | 205 | 212 | 212 | 307 |
| 5679 | 83 | 211 | 212 | 212 | 303 |
| 5680 | 86 | 212 | 212 | 212 | 301 |
| 5681 | 68 | 204 | 212 | 212 | 304 |
| 5682 | 109 | 201 | 212 | 212 | 301 |
| 5683 | 12 | 207 | 201 | 201 | 303 |
| 5684 | 79 | 206 | 212 | 212 | 301 |
| 5685 | 30 | 210 | 201 | 201 | 305 |
| 5686 | 96 | 212 | 201 | 201 | 301 |
| 5687 | 71 | 211 | 202 | 212 | 306 |
| 5688 | 70 | 205 | 201 | 201 | 302 |
| 5689 | 88 | 206 | 202 | 212 | 303 |
| 5690 | 44 | 202 | 201 | 201 | 307 |
| 5691 | 11 | 212 | 212 | 212 | 306 |
| 5692 | 121 | 209 | 202 | 212 | 308 |
| 5693 | 96 | 209 | 201 | 201 | 302 |
| 5694 | 53 | 204 | 201 | 201 | 303 |
| 5695 | 4 | 201 | 201 | 201 | 304 |
| 5696 | 48 | 212 | 202 | 212 | 303 |
| 5697 | 94 | 201 | 212 | 212 | 304 |
| 5698 | 98 | 208 | 201 | 201 | 301 |
| 5699 | 9 | 212 | 212 | 212 | 305 |
| 5700 | 72 | 204 | 202 | 212 | 305 |
| 5701 | 109 | 204 | 212 | 212 | 303 |
| 5702 | 25 | 204 | 202 | 212 | 306 |
| 5703 | 85 | 211 | 212 | 212 | 301 |
| 5704 | 60 | 202 | 201 | 201 | 301 |
| 5705 | 76 | 202 | 202 | 212 | 306 |
| 5706 | 34 | 205 | 201 | 201 | 304 |
| 5707 | 91 | 212 | 201 | 201 | 304 |
| 5708 | 22 | 211 | 201 | 201 | 308 |
| 5709 | 24 | 204 | 201 | 201 | 301 |
| 5710 | 50 | 212 | 202 | 212 | 307 |
| 5711 | 91 | 210 | 201 | 201 | 308 |
| 5712 | 96 | 210 | 212 | 212 | 305 |
| 5713 | 88 | 210 | 202 | 212 | 306 |
| 5714 | 96 | 201 | 201 | 201 | 304 |
| 5715 | 56 | 209 | 202 | 212 | 304 |
| 5716 | 91 | 209 | 212 | 212 | 302 |
| 5717 | 74 | 210 | 201 | 201 | 303 |
| 5718 | 89 | 206 | 202 | 212 | 306 |
| 5719 | 85 | 204 | 202 | 212 | 301 |
| 5720 | 97 | 210 | 201 | 201 | 304 |
| 5721 | 117 | 211 | 202 | 212 | 302 |
| 5722 | 57 | 202 | 212 | 212 | 308 |
| 5723 | 45 | 205 | 201 | 201 | 303 |
| 5724 | 90 | 205 | 212 | 212 | 305 |
| 5725 | 100 | 212 | 212 | 212 | 308 |
| 5726 | 108 | 205 | 212 | 212 | 302 |
| 5727 | 125 | 201 | 212 | 212 | 308 |
| 5728 | 67 | 211 | 201 | 201 | 302 |
| 5729 | 14 | 203 | 201 | 201 | 308 |
| 5730 | 5 | 205 | 201 | 201 | 302 |
| 5731 | 1 | 201 | 212 | 212 | 308 |
| 5732 | 66 | 201 | 212 | 212 | 304 |
| 5733 | 98 | 209 | 201 | 201 | 307 |
| 5734 | 69 | 210 | 201 | 201 | 303 |
| 5735 | 113 | 210 | 201 | 201 | 307 |
| 5736 | 65 | 212 | 212 | 212 | 306 |
| 5737 | 85 | 204 | 212 | 212 | 308 |
| 5738 | 33 | 203 | 202 | 212 | 302 |
| 5739 | 20 | 201 | 201 | 201 | 305 |
| 5740 | 116 | 205 | 202 | 212 | 301 |
| 5741 | 41 | 202 | 202 | 212 | 302 |
| 5742 | 98 | 205 | 201 | 201 | 302 |
| 5743 | 101 | 211 | 201 | 201 | 306 |
| 5744 | 13 | 204 | 201 | 201 | 302 |
| 5745 | 78 | 204 | 201 | 201 | 307 |
| 5746 | 16 | 205 | 201 | 201 | 306 |
| 5747 | 8 | 204 | 212 | 212 | 301 |
| 5748 | 98 | 208 | 201 | 201 | 308 |
| 5749 | 93 | 205 | 212 | 212 | 307 |
| 5750 | 96 | 211 | 201 | 201 | 301 |
| 5751 | 107 | 204 | 201 | 201 | 302 |
| 5752 | 19 | 202 | 201 | 201 | 308 |
| 5753 | 35 | 209 | 202 | 212 | 301 |
| 5754 | 62 | 205 | 212 | 212 | 303 |
| 5755 | 120 | 201 | 201 | 201 | 304 |
| 5756 | 96 | 212 | 212 | 212 | 307 |
| 5757 | 47 | 203 | 202 | 212 | 306 |
| 5758 | 122 | 201 | 201 | 201 | 308 |
| 5759 | 64 | 212 | 201 | 201 | 308 |
| 5760 | 87 | 206 | 202 | 212 | 302 |
| 5761 | 104 | 210 | 202 | 212 | 307 |
| 5762 | 51 | 211 | 201 | 201 | 307 |
| 5763 | 127 | 210 | 201 | 201 | 301 |
| 5764 | 111 | 204 | 202 | 212 | 308 |
| 5765 | 85 | 204 | 212 | 212 | 301 |
| 5766 | 37 | 204 | 202 | 212 | 306 |
| 5767 | 91 | 206 | 212 | 212 | 307 |
| 5768 | 109 | 207 | 201 | 201 | 304 |
| 5769 | 85 | 212 | 212 | 212 | 307 |
| 5770 | 32 | 211 | 212 | 212 | 301 |
| 5771 | 21 | 212 | 202 | 212 | 301 |
| 5772 | 86 | 210 | 212 | 212 | 303 |
| 5773 | 10 | 211 | 202 | 212 | 308 |
| 5774 | 88 | 206 | 212 | 212 | 301 |
| 5775 | 95 | 204 | 201 | 201 | 308 |
| 5776 | 115 | 206 | 201 | 201 | 308 |
| 5777 | 75 | 204 | 202 | 212 | 302 |
| 5778 | 92 | 204 | 212 | 212 | 301 |
| 5779 | 31 | 208 | 212 | 212 | 305 |
| 5780 | 49 | 209 | 201 | 201 | 307 |
| 5781 | 109 | 207 | 201 | 201 | 306 |
| 5782 | 109 | 212 | 201 | 201 | 305 |
| 5783 | 101 | 205 | 202 | 212 | 302 |
| 5784 | 110 | 207 | 201 | 201 | 302 |
| 5785 | 101 | 207 | 212 | 212 | 301 |
| 5786 | 42 | 205 | 212 | 212 | 308 |
| 5787 | 101 | 201 | 212 | 212 | 308 |
| 5788 | 6 | 210 | 202 | 212 | 306 |
| 5789 | 98 | 212 | 201 | 201 | 307 |
| 5790 | 40 | 210 | 202 | 212 | 306 |
| 5791 | 88 | 204 | 202 | 212 | 306 |
| 5792 | 114 | 212 | 202 | 212 | 304 |
| 5793 | 91 | 201 | 201 | 201 | 305 |
| 5794 | 98 | 204 | 212 | 212 | 301 |
| 5795 | 3 | 203 | 202 | 212 | 302 |
| 5796 | 36 | 212 | 201 | 201 | 301 |
| 5797 | 58 | 202 | 212 | 212 | 306 |
| 5798 | 38 | 206 | 201 | 201 | 305 |
| 5799 | 102 | 201 | 201 | 201 | 308 |
| 5800 | 86 | 201 | 202 | 212 | 301 |
| 5801 | 101 | 212 | 202 | 212 | 307 |
| 5802 | 28 | 212 | 202 | 212 | 302 |
| 5803 | 96 | 204 | 201 | 201 | 308 |
| 5804 | 43 | 212 | 202 | 212 | 308 |
| 5805 | 103 | 205 | 201 | 201 | 301 |
| 5806 | 23 | 205 | 202 | 212 | 301 |
| 5807 | 112 | 212 | 201 | 201 | 304 |
| 5808 | 18 | 202 | 202 | 212 | 306 |
| 5809 | 81 | 210 | 212 | 212 | 301 |
| 5810 | 59 | 206 | 201 | 201 | 306 |
| 5811 | 119 | 212 | 212 | 212 | 302 |
| 5812 | 96 | 201 | 201 | 201 | 308 |
| 5813 | 2 | 201 | 201 | 201 | 308 |
| 5814 | 126 | 212 | 202 | 212 | 307 |
| 5815 | 86 | 201 | 212 | 212 | 302 |
| 5816 | 39 | 204 | 212 | 212 | 302 |
| 5817 | 77 | 204 | 212 | 212 | 301 |
| 5818 | 129 | 211 | 202 | 212 | 302 |
| 5819 | 26 | 209 | 212 | 212 | 303 |
| 5820 | 86 | 204 | 212 | 212 | 304 |
| 5821 | 84 | 203 | 212 | 212 | 303 |
| 5822 | 91 | 203 | 201 | 201 | 306 |
| 5823 | 118 | 203 | 212 | 212 | 306 |
| 5824 | 99 | 203 | 202 | 212 | 307 |
| 5825 | 123 | 201 | 201 | 201 | 307 |

Compounds having the formula:

TABLE 34

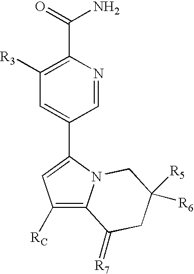

wherein R₃, R_{C}, R₅, R₆, and R₇ are defined in Table 34:

| Compound | | | | | |
|---|---|---|---|---|---|
| No. | R₃ | R_{C} | R₅ | R₆ | R₇ |
| 5826 | 9 | 212 | 212 | 212 | 305 |
| 5827 | 98 | 205 | 201 | 201 | 302 |
| 5828 | 89 | 206 | 202 | 212 | 306 |
| 5829 | 15 | 211 | 212 | 212 | 302 |
| 5830 | 91 | 210 | 201 | 201 | 308 |
| 5831 | 33 | 203 | 202 | 212 | 302 |
| 5832 | 126 | 212 | 202 | 212 | 307 |
| 5833 | 22 | 211 | 201 | 201 | 308 |
| 5834 | 109 | 207 | 201 | 201 | 304 |
| 5835 | 10 | 211 | 202 | 212 | 308 |
| 5836 | 100 | 212 | 212 | 212 | 308 |
| 5837 | 29 | 210 | 202 | 212 | 306 |
| 5838 | 30 | 210 | 201 | 201 | 305 |
| 5839 | 119 | 212 | 212 | 212 | 302 |
| 5840 | 109 | 212 | 201 | 201 | 305 |
| 5841 | 67 | 211 | 201 | 201 | 302 |
| 5842 | 42 | 205 | 212 | 212 | 308 |
| 5843 | 101 | 205 | 202 | 212 | 302 |
| 5844 | 32 | 211 | 212 | 212 | 301 |
| 5845 | 19 | 202 | 201 | 201 | 308 |
| 5846 | 68 | 204 | 212 | 212 | 304 |
| 5847 | 104 | 210 | 202 | 212 | 307 |
| 5848 | 96 | 212 | 201 | 201 | 301 |
| 5849 | 31 | 208 | 212 | 212 | 305 |
| 5850 | 88 | 210 | 201 | 201 | 301 |
| 5851 | 83 | 211 | 212 | 212 | 303 |
| 5852 | 7 | 204 | 201 | 201 | 307 |
| 5853 | 23 | 205 | 202 | 212 | 301 |
| 5854 | 63 | 210 | 202 | 212 | 308 |
| 5855 | 121 | 209 | 202 | 212 | 308 |
| 5856 | 86 | 201 | 202 | 212 | 301 |
| 5857 | 78 | 204 | 201 | 201 | 307 |
| 5858 | 77 | 204 | 212 | 212 | 301 |
| 5859 | 21 | 212 | 202 | 212 | 301 |
| 5860 | 115 | 206 | 201 | 201 | 308 |
| 5861 | 50 | 212 | 202 | 212 | 307 |
| 5862 | 27 | 212 | 202 | 212 | 306 |
| 5863 | 88 | 206 | 202 | 212 | 303 |
| 5864 | 96 | 204 | 201 | 201 | 308 |
| 5865 | 98 | 208 | 201 | 201 | 308 |
| 5866 | 4 | 201 | 201 | 201 | 304 |
| 5867 | 86 | 205 | 201 | 201 | 303 |
| 5868 | 17 | 207 | 201 | 201 | 308 |
| 5869 | 35 | 209 | 202 | 212 | 301 |
| 5870 | 90 | 205 | 212 | 212 | 305 |
| 5871 | 101 | 211 | 201 | 201 | 306 |
| 5872 | 69 | 210 | 201 | 201 | 303 |
| 5873 | 110 | 207 | 201 | 201 | 302 |
| 5874 | 84 | 203 | 212 | 212 | 303 |
| 5875 | 98 | 209 | 201 | 201 | 307 |
| 5876 | 118 | 203 | 212 | 212 | 306 |
| 5877 | 46 | 204 | 202 | 212 | 308 |
| 5878 | 111 | 204 | 202 | 212 | 308 |
| 5879 | 60 | 202 | 201 | 201 | 301 |
| 5880 | 25 | 204 | 202 | 212 | 306 |
| 5881 | 5 | 205 | 201 | 201 | 302 |
| 5882 | 101 | 212 | 202 | 212 | 307 |
| 5883 | 109 | 201 | 212 | 212 | 301 |
| 5884 | 71 | 211 | 202 | 212 | 306 |
| 5885 | 88 | 204 | 202 | 212 | 306 |
| 5886 | 13 | 204 | 201 | 201 | 302 |
| 5887 | 73 | 210 | 202 | 212 | 308 |
| 5888 | 99 | 203 | 202 | 212 | 307 |
| 5889 | 97 | 210 | 201 | 201 | 304 |
| 5890 | 92 | 204 | 212 | 212 | 301 |
| 5891 | 2 | 201 | 201 | 201 | 308 |
| 5892 | 96 | 210 | 212 | 212 | 305 |
| 5893 | 53 | 204 | 201 | 201 | 303 |
| 5894 | 41 | 202 | 202 | 212 | 302 |
| 5895 | 127 | 210 | 201 | 201 | 301 |
| 5896 | 76 | 202 | 202 | 212 | 306 |
| 5897 | 62 | 205 | 212 | 212 | 303 |
| 5898 | 43 | 212 | 202 | 212 | 308 |
| 5899 | 103 | 205 | 201 | 201 | 301 |
| 5900 | 16 | 205 | 201 | 201 | 306 |
| 5901 | 54 | 212 | 201 | 201 | 302 |
| 5902 | 58 | 202 | 212 | 212 | 306 |
| 5903 | 86 | 212 | 212 | 212 | 301 |
| 5904 | 91 | 201 | 201 | 201 | 305 |
| 5905 | 39 | 204 | 212 | 212 | 302 |
| 5906 | 36 | 212 | 201 | 201 | 301 |
| 5907 | 45 | 205 | 201 | 201 | 303 |
| 5908 | 130 | 206 | 202 | 212 | 306 |
| 5909 | 128 | 212 | 201 | 201 | 303 |
| 5910 | 117 | 211 | 202 | 212 | 302 |
| 5911 | 107 | 204 | 201 | 201 | 302 |
| 5912 | 93 | 205 | 212 | 212 | 307 |
| 5913 | 91 | 209 | 212 | 212 | 302 |
| 5914 | 114 | 212 | 202 | 212 | 304 |
| 5915 | 11 | 212 | 212 | 212 | 306 |
| 5916 | 108 | 205 | 212 | 212 | 302 |
| 5917 | 94 | 201 | 212 | 212 | 304 |
| 5918 | 85 | 204 | 212 | 212 | 301 |
| 5919 | 116 | 205 | 202 | 212 | 301 |
| 5920 | 52 | 201 | 202 | 212 | 306 |
| 5921 | 28 | 212 | 202 | 212 | 302 |
| 5922 | 105 | 212 | 202 | 212 | 306 |
| 5923 | 47 | 203 | 202 | 212 | 306 |
| 5924 | 101 | 207 | 212 | 212 | 301 |
| 5925 | 112 | 212 | 201 | 201 | 304 |
| 5926 | 38 | 206 | 201 | 201 | 305 |
| 5927 | 86 | 204 | 212 | 212 | 304 |
| 5928 | 12 | 207 | 201 | 201 | 303 |
| 5929 | 123 | 201 | 201 | 201 | 307 |
| 5930 | 18 | 202 | 202 | 212 | 306 |
| 5931 | 64 | 212 | 201 | 201 | 308 |
| 5932 | 95 | 204 | 201 | 201 | 308 |
| 5933 | 82 | 209 | 202 | 212 | 302 |
| 5934 | 109 | 204 | 212 | 212 | 303 |
| 5935 | 74 | 210 | 201 | 201 | 303 |
| 5936 | 85 | 211 | 212 | 212 | 301 |
| 5937 | 55 | 204 | 201 | 201 | 302 |
| 5938 | 66 | 201 | 212 | 212 | 304 |
| 5939 | 85 | 212 | 212 | 212 | 307 |
| 5940 | 91 | 203 | 201 | 201 | 306 |
| 5941 | 98 | 204 | 212 | 212 | 301 |
| 5942 | 88 | 206 | 212 | 212 | 301 |
| 5943 | 3 | 203 | 202 | 212 | 302 |
| 5944 | 8 | 204 | 212 | 212 | 301 |
| 5945 | 59 | 206 | 201 | 201 | 306 |
| 5946 | 96 | 201 | 201 | 201 | 304 |
| 5947 | 6 | 210 | 202 | 212 | 306 |
| 5948 | 14 | 203 | 201 | 201 | 308 |
| 5949 | 122 | 201 | 201 | 201 | 308 |
| 5950 | 96 | 212 | 212 | 212 | 307 |
| 5951 | 51 | 211 | 201 | 201 | 307 |
| 5952 | 85 | 210 | 202 | 212 | 303 |
| 5953 | 129 | 211 | 202 | 212 | 302 |
| 5954 | 85 | 204 | 202 | 212 | 301 |
| 5955 | 37 | 204 | 202 | 212 | 306 |
| 5956 | 87 | 206 | 202 | 212 | 302 |
| 5957 | 40 | 210 | 202 | 212 | 306 |
| 5958 | 20 | 201 | 201 | 201 | 305 |
| 5959 | 98 | 212 | 201 | 201 | 307 |
| 5960 | 88 | 208 | 212 | 212 | 301 |
| 5961 | 124 | 203 | 201 | 201 | 308 |
| 5962 | 56 | 209 | 202 | 212 | 304 |
| 5963 | 80 | 204 | 212 | 212 | 306 |
| 5964 | 86 | 210 | 212 | 212 | 303 |
| 5965 | 109 | 207 | 201 | 201 | 306 |
| 5966 | 113 | 210 | 201 | 201 | 307 |
| 5967 | 96 | 211 | 201 | 201 | 301 |
| 5968 | 88 | 211 | 201 | 201 | 306 |
| 5969 | 106 | 205 | 212 | 212 | 307 |
| 5970 | 65 | 212 | 212 | 212 | 306 |
| 5971 | 48 | 212 | 202 | 212 | 303 |
| 5972 | 125 | 201 | 212 | 212 | 308 |
| 5973 | 44 | 202 | 201 | 201 | 307 |
| 5974 | 85 | 204 | 212 | 212 | 308 |
| 5975 | 96 | 209 | 201 | 201 | 302 |
| 5976 | 88 | 209 | 212 | 212 | 307 |
| 5977 | 120 | 201 | 201 | 201 | 304 |
| 5978 | 102 | 201 | 201 | 201 | 308 |
| 5979 | 91 | 212 | 201 | 201 | 304 |
| 5980 | 101 | 203 | 201 | 201 | 301 |
| 5981 | 109 | 204 | 201 | 201 | 303 |
| 5982 | 86 | 201 | 212 | 212 | 302 |
| 5983 | 101 | 201 | 212 | 212 | 308 |
| 5984 | 91 | 206 | 212 | 212 | 307 |
| 5985 | 96 | 201 | 201 | 201 | 308 |
| 5986 | 24 | 204 | 201 | 201 | 301 |
| 5987 | 72 | 204 | 202 | 212 | 305 |
| 5988 | 75 | 204 | 202 | 212 | 302 |
| 5989 | 1 | 201 | 212 | 212 | 308 |
| 5990 | 49 | 209 | 201 | 201 | 307 |
| 5991 | 61 | 202 | 202 | 212 | 301 |
| 5992 | 109 | 207 | 201 | 201 | 302 |
| 5993 | 88 | 210 | 202 | 212 | 306 |
| 5994 | 26 | 209 | 212 | 212 | 303 |
| 5995 | 79 | 206 | 212 | 212 | 301 |
| 5996 | 81 | 210 | 212 | 212 | 301 |
| 5997 | 70 | 205 | 201 | 201 | 302 |
| 5998 | 57 | 202 | 212 | 212 | 308 |
| 5999 | 98 | 208 | 201 | 201 | 301 |
| 6000 | 34 | 205 | 201 | 201 | 304 |

Compounds having the formula:

TABLE 35

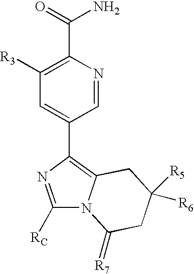

wherein R₃, R_{C}, R₅, R₆, and R₇ are defined in Table 35:

| Compound | | | | | |
|---|---|---|---|---|---|
| No. | R₃ | R_{C} | R₅ | R₆ | R₇ |
| 6001 | 98 | 205 | 201 | 201 | 302 |
| 6002 | 40 | 210 | 202 | 212 | 306 |
| 6003 | 115 | 206 | 201 | 201 | 308 |
| 6004 | 125 | 201 | 212 | 212 | 308 |
| 6005 | 130 | 206 | 202 | 212 | 306 |
| 6006 | 91 | 212 | 201 | 201 | 304 |
| 6007 | 96 | 210 | 212 | 212 | 305 |
| 6008 | 96 | 211 | 201 | 201 | 301 |
| 6009 | 31 | 208 | 212 | 212 | 305 |
| 6010 | 120 | 201 | 201 | 201 | 304 |
| 6011 | 100 | 212 | 212 | 212 | 308 |
| 6012 | 97 | 210 | 201 | 201 | 304 |
| 6013 | 96 | 209 | 201 | 201 | 302 |
| 6014 | 118 | 203 | 212 | 212 | 306 |
| 6015 | 88 | 210 | 201 | 201 | 301 |
| 6016 | 69 | 210 | 201 | 201 | 303 |
| 6017 | 88 | 208 | 212 | 212 | 301 |
| 6018 | 98 | 208 | 201 | 201 | 308 |
| 6019 | 85 | 204 | 202 | 212 | 301 |
| 6020 | 35 | 209 | 202 | 212 | 301 |
| 6021 | 52 | 201 | 202 | 212 | 306 |
| 6022 | 106 | 205 | 212 | 212 | 307 |
| 6023 | 85 | 211 | 212 | 212 | 301 |
| 6024 | 96 | 204 | 201 | 201 | 308 |
| 6025 | 50 | 212 | 202 | 212 | 307 |
| 6026 | 67 | 211 | 201 | 201 | 302 |
| 6027 | 71 | 211 | 202 | 212 | 306 |
| 6028 | 45 | 205 | 201 | 201 | 303 |
| 6029 | 38 | 206 | 201 | 201 | 305 |
| 6030 | 96 | 212 | 212 | 212 | 307 |
| 6031 | 34 | 205 | 201 | 201 | 304 |
| 6032 | 68 | 204 | 212 | 212 | 304 |
| 6033 | 129 | 211 | 202 | 212 | 302 |
| 6034 | 102 | 201 | 201 | 201 | 308 |
| 6035 | 109 | 207 | 201 | 201 | 306 |
| 6036 | 30 | 210 | 201 | 201 | 305 |
| 6037 | 48 | 212 | 202 | 212 | 303 |
| 6038 | 79 | 206 | 212 | 212 | 301 |
| 6039 | 32 | 211 | 212 | 212 | 301 |
| 6040 | 46 | 204 | 202 | 212 | 308 |
| 6041 | 20 | 201 | 201 | 201 | 305 |
| 6042 | 7 | 204 | 201 | 201 | 307 |
| 6043 | 98 | 209 | 201 | 201 | 307 |
| 6044 | 74 | 210 | 201 | 201 | 303 |
| 6045 | 108 | 205 | 212 | 212 | 302 |
| 6046 | 111 | 204 | 202 | 212 | 308 |
| 6047 | 61 | 202 | 202 | 212 | 301 |
| 6048 | 16 | 205 | 201 | 201 | 306 |
| 6049 | 14 | 203 | 201 | 201 | 308 |
| 6050 | 43 | 212 | 202 | 212 | 308 |
| 6051 | 49 | 209 | 201 | 201 | 307 |
| 6052 | 93 | 205 | 212 | 212 | 307 |
| 6053 | 10 | 211 | 202 | 212 | 308 |
| 6054 | 78 | 204 | 201 | 201 | 307 |
| 6055 | 92 | 204 | 212 | 212 | 301 |
| 6056 | 85 | 212 | 212 | 212 | 307 |
| 6057 | 13 | 204 | 201 | 201 | 302 |
| 6058 | 62 | 205 | 212 | 212 | 303 |
| 6059 | 15 | 211 | 212 | 212 | 302 |
| 6060 | 89 | 206 | 202 | 212 | 306 |
| 6061 | 76 | 202 | 202 | 212 | 306 |
| 6062 | 109 | 207 | 201 | 201 | 302 |
| 6063 | 95 | 204 | 201 | 201 | 308 |
| 6064 | 29 | 210 | 202 | 212 | 306 |
| 6065 | 12 | 207 | 201 | 201 | 303 |
| 6066 | 116 | 205 | 202 | 212 | 301 |
| 6067 | 77 | 204 | 212 | 212 | 301 |
| 6068 | 88 | 206 | 212 | 212 | 301 |
| 6069 | 119 | 212 | 212 | 212 | 302 |
| 6070 | 101 | 211 | 201 | 201 | 306 |
| 6071 | 1 | 201 | 212 | 212 | 308 |
| 6072 | 110 | 207 | 201 | 201 | 302 |
| 6073 | 17 | 207 | 201 | 201 | 308 |
| 6074 | 86 | 205 | 201 | 201 | 303 |
| 6075 | 27 | 212 | 202 | 212 | 306 |
| 6076 | 22 | 211 | 201 | 201 | 308 |
| 6077 | 96 | 201 | 201 | 201 | 308 |
| 6078 | 101 | 207 | 212 | 212 | 301 |
| 6079 | 81 | 210 | 212 | 212 | 301 |
| 6080 | 91 | 206 | 212 | 212 | 307 |
| 6081 | 21 | 212 | 202 | 212 | 301 |
| 6082 | 6 | 210 | 202 | 212 | 306 |
| 6083 | 56 | 209 | 202 | 212 | 304 |
| 6084 | 109 | 207 | 201 | 201 | 304 |
| 6085 | 101 | 212 | 202 | 212 | 307 |
| 6086 | 99 | 203 | 202 | 212 | 307 |
| 6087 | 63 | 210 | 202 | 212 | 308 |
| 6088 | 105 | 212 | 202 | 212 | 306 |
| 6089 | 57 | 202 | 212 | 212 | 308 |
| 6090 | 101 | 205 | 202 | 212 | 302 |
| 6091 | 24 | 204 | 201 | 201 | 301 |
| 6092 | 18 | 202 | 202 | 212 | 306 |
| 6093 | 94 | 201 | 212 | 212 | 304 |
| 6094 | 25 | 204 | 202 | 212 | 306 |
| 6095 | 28 | 212 | 202 | 212 | 302 |
| 6096 | 58 | 202 | 212 | 212 | 306 |
| 6097 | 86 | 204 | 212 | 212 | 304 |
| 6098 | 114 | 212 | 202 | 212 | 304 |
| 6099 | 90 | 205 | 212 | 212 | 305 |
| 6100 | 112 | 212 | 201 | 201 | 304 |
| 6101 | 42 | 205 | 212 | 212 | 308 |
| 6102 | 2 | 201 | 201 | 201 | 308 |
| 6103 | 82 | 209 | 202 | 212 | 302 |
| 6104 | 59 | 206 | 201 | 201 | 306 |
| 6105 | 113 | 210 | 201 | 201 | 307 |
| 6106 | 83 | 211 | 212 | 212 | 303 |
| 6107 | 37 | 204 | 202 | 212 | 306 |
| 6108 | 3 | 203 | 202 | 212 | 302 |
| 6109 | 86 | 201 | 212 | 212 | 302 |
| 6110 | 55 | 204 | 201 | 201 | 302 |
| 6111 | 86 | 201 | 202 | 212 | 301 |
| 6112 | 126 | 212 | 202 | 212 | 307 |
| 6113 | 109 | 212 | 201 | 201 | 305 |
| 6114 | 80 | 204 | 212 | 212 | 306 |
| 6115 | 8 | 204 | 212 | 212 | 301 |
| 6116 | 124 | 203 | 201 | 201 | 308 |
| 6117 | 91 | 210 | 201 | 201 | 308 |
| 6118 | 85 | 204 | 212 | 212 | 308 |
| 6119 | 87 | 206 | 202 | 212 | 302 |
| 6120 | 96 | 212 | 201 | 201 | 301 |
| 6121 | 88 | 210 | 202 | 212 | 306 |
| 6122 | 72 | 204 | 202 | 212 | 305 |
| 6123 | 39 | 204 | 212 | 212 | 302 |
| 6124 | 88 | 211 | 201 | 201 | 306 |
| 6125 | 104 | 210 | 202 | 212 | 307 |
| 6126 | 91 | 203 | 201 | 201 | 306 |
| 6127 | 4 | 201 | 201 | 201 | 304 |
| 6128 | 65 | 212 | 212 | 212 | 306 |
| 6129 | 64 | 212 | 201 | 201 | 308 |
| 6130 | 98 | 208 | 201 | 201 | 301 |
| 6131 | 70 | 205 | 201 | 201 | 302 |
| 6132 | 88 | 204 | 202 | 212 | 306 |
| 6133 | 47 | 203 | 202 | 212 | 306 |
| 6134 | 66 | 201 | 212 | 212 | 304 |
| 6135 | 44 | 202 | 201 | 201 | 307 |
| 6136 | 123 | 201 | 201 | 201 | 307 |
| 6137 | 128 | 212 | 201 | 201 | 303 |
| 6138 | 53 | 204 | 201 | 201 | 303 |
| 6139 | 127 | 210 | 201 | 201 | 301 |
| 6140 | 33 | 203 | 202 | 212 | 302 |
| 6141 | 51 | 211 | 201 | 201 | 307 |
| 6142 | 101 | 203 | 201 | 201 | 301 |
| 6143 | 88 | 206 | 202 | 212 | 303 |
| 6144 | 88 | 209 | 212 | 212 | 307 |
| 6145 | 109 | 204 | 212 | 212 | 303 |
| 6146 | 98 | 212 | 201 | 201 | 307 |
| 6147 | 60 | 202 | 201 | 201 | 301 |
| 6148 | 98 | 204 | 212 | 212 | 301 |
| 6149 | 11 | 212 | 212 | 212 | 306 |
| 6150 | 109 | 204 | 201 | 201 | 303 |
| 6151 | 86 | 210 | 212 | 212 | 303 |
| 6152 | 84 | 203 | 212 | 212 | 303 |
| 6153 | 96 | 201 | 201 | 201 | 304 |
| 6154 | 101 | 201 | 212 | 212 | 308 |
| 6155 | 109 | 201 | 212 | 212 | 301 |
| 6156 | 5 | 205 | 201 | 201 | 302 |
| 6157 | 41 | 202 | 202 | 212 | 302 |
| 6158 | 91 | 209 | 212 | 212 | 302 |
| 6159 | 19 | 202 | 201 | 201 | 308 |
| 6160 | 9 | 212 | 212 | 212 | 305 |
| 6161 | 103 | 205 | 201 | 201 | 301 |
| 6162 | 85 | 204 | 212 | 212 | 301 |
| 6163 | 36 | 212 | 201 | 201 | 301 |
| 6164 | 73 | 210 | 202 | 212 | 308 |
| 6165 | 86 | 212 | 212 | 212 | 301 |
| 6166 | 117 | 211 | 202 | 212 | 302 |
| 6167 | 85 | 210 | 202 | 212 | 303 |
| 6168 | 107 | 204 | 201 | 201 | 302 |
| 6169 | 26 | 209 | 212 | 212 | 303 |
| 6170 | 75 | 204 | 202 | 212 | 302 |
| 6171 | 91 | 201 | 201 | 201 | 305 |
| 6172 | 122 | 201 | 201 | 201 | 308 |
| 6173 | 54 | 212 | 201 | 201 | 302 |
| 6174 | 121 | 209 | 202 | 212 | 308 |
| 6175 | 23 | 205 | 202 | 212 | 301 |

Compounds having the formula:

TABLE 36

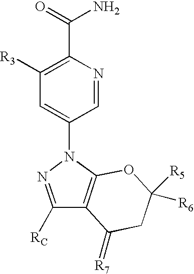

wherein R₃, R_{C}, R₅, R₆, and R₇ are defined in Table 36:

| Compound | | | | | |
|---|---|---|---|---|---|
| No. | R₃ | R_{C} | R₅ | R₆ | R₇ |
| 6176 | 96 | 209 | 201 | 201 | 302 |
| 6177 | 109 | 204 | 201 | 201 | 303 |
| 6178 | 5 | 205 | 201 | 201 | 302 |
| 6179 | 48 | 212 | 202 | 212 | 303 |
| 6180 | 56 | 209 | 202 | 212 | 304 |
| 6181 | 87 | 206 | 202 | 212 | 302 |
| 6182 | 73 | 210 | 202 | 212 | 308 |
| 6183 | 71 | 211 | 202 | 212 | 306 |
| 6184 | 96 | 204 | 201 | 201 | 308 |
| 6185 | 88 | 204 | 202 | 212 | 306 |
| 6186 | 26 | 209 | 212 | 212 | 303 |
| 6187 | 109 | 212 | 201 | 201 | 305 |
| 6188 | 72 | 204 | 202 | 212 | 305 |
| 6189 | 24 | 204 | 201 | 201 | 301 |
| 6190 | 101 | 211 | 201 | 201 | 306 |
| 6191 | 49 | 209 | 201 | 201 | 307 |
| 6192 | 86 | 205 | 201 | 201 | 303 |
| 6193 | 11 | 212 | 212 | 212 | 306 |
| 6194 | 46 | 204 | 202 | 212 | 308 |
| 6195 | 74 | 210 | 201 | 201 | 303 |
| 6196 | 97 | 210 | 201 | 201 | 304 |
| 6197 | 86 | 201 | 202 | 212 | 301 |
| 6198 | 98 | 212 | 201 | 201 | 307 |
| 6199 | 85 | 204 | 212 | 212 | 308 |
| 6200 | 45 | 205 | 201 | 201 | 303 |
| 6201 | 88 | 210 | 202 | 212 | 306 |
| 6202 | 62 | 205 | 212 | 212 | 303 |
| 6203 | 10 | 211 | 202 | 212 | 308 |
| 6204 | 108 | 205 | 212 | 212 | 302 |
| 6205 | 96 | 211 | 201 | 201 | 301 |
| 6206 | 96 | 201 | 201 | 201 | 308 |
| 6207 | 127 | 210 | 201 | 201 | 301 |
| 6208 | 109 | 207 | 201 | 201 | 302 |
| 6209 | 21 | 212 | 202 | 212 | 301 |
| 6210 | 33 | 203 | 202 | 212 | 302 |
| 6211 | 6 | 210 | 202 | 212 | 306 |
| 6212 | 85 | 211 | 212 | 212 | 301 |
| 6213 | 102 | 201 | 201 | 201 | 308 |
| 6214 | 59 | 206 | 201 | 201 | 306 |
| 6215 | 4 | 201 | 201 | 201 | 304 |
| 6216 | 114 | 212 | 202 | 212 | 304 |
| 6217 | 124 | 203 | 201 | 201 | 308 |
| 6218 | 43 | 212 | 202 | 212 | 308 |
| 6219 | 55 | 204 | 201 | 201 | 302 |
| 6220 | 37 | 204 | 202 | 212 | 306 |
| 6221 | 98 | 205 | 201 | 201 | 302 |
| 6222 | 91 | 209 | 212 | 212 | 302 |
| 6223 | 112 | 212 | 201 | 201 | 304 |
| 6224 | 39 | 204 | 212 | 212 | 302 |
| 6225 | 19 | 202 | 201 | 201 | 308 |
| 6226 | 57 | 202 | 212 | 212 | 308 |
| 6227 | 119 | 212 | 212 | 212 | 302 |
| 6228 | 40 | 210 | 202 | 212 | 306 |
| 6229 | 86 | 210 | 212 | 212 | 303 |
| 6230 | 64 | 212 | 201 | 201 | 308 |
| 6231 | 94 | 201 | 212 | 212 | 304 |
| 6232 | 76 | 202 | 202 | 212 | 306 |
| 6233 | 104 | 210 | 202 | 212 | 307 |
| 6234 | 109 | 207 | 201 | 201 | 306 |
| 6235 | 17 | 207 | 201 | 201 | 308 |
| 6236 | 12 | 207 | 201 | 201 | 303 |
| 6237 | 98 | 208 | 201 | 201 | 308 |
| 6238 | 90 | 205 | 212 | 212 | 305 |
| 6239 | 78 | 204 | 201 | 201 | 307 |
| 6240 | 25 | 204 | 202 | 212 | 306 |
| 6241 | 82 | 209 | 202 | 212 | 302 |
| 6242 | 75 | 204 | 202 | 212 | 302 |
| 6243 | 91 | 201 | 201 | 201 | 305 |
| 6244 | 69 | 210 | 201 | 201 | 303 |
| 6245 | 9 | 212 | 212 | 212 | 305 |
| 6246 | 79 | 206 | 212 | 212 | 301 |
| 6247 | 15 | 211 | 212 | 212 | 302 |
| 6248 | 98 | 209 | 201 | 201 | 307 |
| 6249 | 123 | 201 | 201 | 201 | 307 |
| 6250 | 27 | 212 | 202 | 212 | 306 |
| 6251 | 109 | 201 | 212 | 212 | 301 |
| 6252 | 50 | 212 | 202 | 212 | 307 |
| 6253 | 54 | 212 | 201 | 201 | 302 |
| 6254 | 117 | 211 | 202 | 212 | 302 |
| 6255 | 51 | 211 | 201 | 201 | 307 |
| 6256 | 83 | 211 | 212 | 212 | 303 |
| 6257 | 120 | 201 | 201 | 201 | 304 |
| 6258 | 128 | 212 | 201 | 201 | 303 |
| 6259 | 31 | 208 | 212 | 212 | 305 |
| 6260 | 13 | 204 | 201 | 201 | 302 |
| 6261 | 118 | 203 | 212 | 212 | 306 |
| 6262 | 86 | 212 | 212 | 212 | 301 |
| 6263 | 86 | 204 | 212 | 212 | 304 |
| 6264 | 101 | 207 | 212 | 212 | 301 |
| 6265 | 85 | 210 | 202 | 212 | 303 |
| 6266 | 42 | 205 | 212 | 212 | 308 |
| 6267 | 113 | 210 | 201 | 201 | 307 |
| 6268 | 14 | 203 | 201 | 201 | 308 |
| 6269 | 36 | 212 | 201 | 201 | 301 |
| 6270 | 52 | 201 | 202 | 212 | 306 |
| 6271 | 23 | 205 | 202 | 212 | 301 |
| 6272 | 2 | 201 | 201 | 201 | 308 |
| 6273 | 38 | 206 | 201 | 201 | 305 |
| 6274 | 98 | 208 | 201 | 201 | 301 |
| 6275 | 100 | 212 | 212 | 212 | 308 |
| 6276 | 103 | 205 | 201 | 201 | 301 |
| 6277 | 77 | 204 | 212 | 212 | 301 |
| 6278 | 3 | 203 | 202 | 212 | 302 |
| 6279 | 106 | 205 | 212 | 212 | 307 |
| 6280 | 91 | 212 | 201 | 201 | 304 |
| 6281 | 86 | 201 | 212 | 212 | 302 |
| 6282 | 88 | 208 | 212 | 212 | 301 |
| 6283 | 129 | 211 | 202 | 212 | 302 |
| 6284 | 47 | 203 | 202 | 212 | 306 |
| 6285 | 126 | 212 | 202 | 212 | 307 |
| 6286 | 88 | 210 | 201 | 201 | 301 |
| 6287 | 116 | 205 | 202 | 212 | 301 |
| 6288 | 66 | 201 | 212 | 212 | 304 |
| 6289 | 53 | 204 | 201 | 201 | 303 |
| 6290 | 91 | 210 | 201 | 201 | 308 |
| 6291 | 60 | 202 | 201 | 201 | 301 |
| 6292 | 61 | 202 | 202 | 212 | 301 |
| 6293 | 65 | 212 | 212 | 212 | 306 |
| 6294 | 70 | 205 | 201 | 201 | 302 |
| 6295 | 28 | 212 | 202 | 212 | 302 |
| 6296 | 99 | 203 | 202 | 212 | 307 |
| 6297 | 84 | 203 | 212 | 212 | 303 |
| 6298 | 91 | 206 | 212 | 212 | 307 |
| 6299 | 88 | 206 | 202 | 212 | 303 |
| 6300 | 98 | 204 | 212 | 212 | 301 |
| 6301 | 35 | 209 | 202 | 212 | 301 |
| 6302 | 107 | 204 | 201 | 201 | 302 |
| 6303 | 101 | 205 | 202 | 212 | 302 |
| 6304 | 85 | 204 | 202 | 212 | 301 |
| 6305 | 121 | 209 | 202 | 212 | 308 |
| 6306 | 122 | 201 | 201 | 201 | 308 |
| 6307 | 115 | 206 | 201 | 201 | 308 |
| 6308 | 63 | 210 | 202 | 212 | 308 |
| 6309 | 34 | 205 | 201 | 201 | 304 |
| 6310 | 16 | 205 | 201 | 201 | 306 |
| 6311 | 18 | 202 | 202 | 212 | 306 |
| 6312 | 109 | 207 | 201 | 201 | 304 |
| 6313 | 88 | 206 | 212 | 212 | 301 |
| 6314 | 58 | 202 | 212 | 212 | 306 |
| 6315 | 8 | 204 | 212 | 212 | 301 |
| 6316 | 130 | 206 | 202 | 212 | 306 |
| 6317 | 96 | 212 | 212 | 212 | 307 |
| 6318 | 1 | 201 | 212 | 212 | 308 |
| 6319 | 96 | 212 | 201 | 201 | 301 |
| 6320 | 96 | 201 | 201 | 201 | 304 |
| 6321 | 68 | 204 | 212 | 212 | 304 |
| 6322 | 29 | 210 | 202 | 212 | 306 |
| 6323 | 110 | 207 | 201 | 201 | 302 |
| 6324 | 109 | 204 | 212 | 212 | 303 |
| 6325 | 85 | 212 | 212 | 212 | 307 |
| 6326 | 88 | 209 | 212 | 212 | 307 |
| 6327 | 85 | 204 | 212 | 212 | 301 |
| 6328 | 67 | 211 | 201 | 201 | 302 |
| 6329 | 81 | 210 | 212 | 212 | 301 |
| 6330 | 7 | 204 | 201 | 201 | 307 |
| 6331 | 80 | 204 | 212 | 212 | 306 |
| 6332 | 93 | 205 | 212 | 212 | 307 |
| 6333 | 105 | 212 | 202 | 212 | 306 |
| 6334 | 91 | 203 | 201 | 201 | 306 |
| 6335 | 30 | 210 | 201 | 201 | 305 |
| 6336 | 22 | 211 | 201 | 201 | 308 |
| 6337 | 89 | 206 | 202 | 212 | 306 |
| 6338 | 96 | 210 | 212 | 212 | 305 |
| 6339 | 92 | 204 | 212 | 212 | 301 |
| 6340 | 95 | 204 | 201 | 201 | 308 |
| 6341 | 101 | 212 | 202 | 212 | 307 |
| 6342 | 41 | 202 | 202 | 212 | 302 |
| 6343 | 20 | 201 | 201 | 201 | 305 |
| 6344 | 125 | 201 | 212 | 212 | 308 |
| 6345 | 32 | 211 | 212 | 212 | 301 |
| 6346 | 101 | 201 | 212 | 212 | 308 |
| 6347 | 88 | 211 | 201 | 201 | 306 |
| 6348 | 101 | 203 | 201 | 201 | 301 |
| 6349 | 44 | 202 | 201 | 201 | 307 |
| 6350 | 111 | 204 | 202 | 212 | 308 |

Compounds having the formula:

TABLE 37

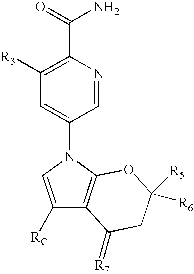

wherein R₃, R_{C}, R₅, R₆, and R₇ are defined in Table 37:

| Compound | | | | | |
|---|---|---|---|---|---|
| No. | R₃ | R_{C} | R₅ | R₆ | R₇ |
| 6351 | 14 | 203 | 201 | 201 | 308 |
| 6352 | 17 | 207 | 201 | 201 | 308 |
| 6353 | 33 | 203 | 202 | 212 | 302 |
| 6354 | 106 | 205 | 212 | 212 | 307 |
| 6355 | 88 | 211 | 201 | 201 | 306 |
| 6356 | 91 | 209 | 212 | 212 | 302 |
| 6357 | 75 | 204 | 202 | 212 | 302 |
| 6358 | 96 | 211 | 201 | 201 | 301 |
| 6359 | 92 | 204 | 212 | 212 | 301 |
| 6360 | 39 | 204 | 212 | 212 | 302 |
| 6361 | 112 | 212 | 201 | 201 | 304 |
| 6362 | 97 | 210 | 201 | 201 | 304 |
| 6363 | 85 | 212 | 212 | 212 | 307 |
| 6364 | 98 | 208 | 201 | 201 | 301 |
| 6365 | 120 | 201 | 201 | 201 | 304 |
| 6366 | 108 | 205 | 212 | 212 | 302 |
| 6367 | 88 | 208 | 212 | 212 | 301 |
| 6368 | 96 | 201 | 201 | 201 | 304 |
| 6369 | 107 | 204 | 201 | 201 | 302 |
| 6370 | 128 | 212 | 201 | 201 | 303 |
| 6371 | 111 | 204 | 202 | 212 | 308 |
| 6372 | 34 | 205 | 201 | 201 | 304 |
| 6373 | 86 | 201 | 202 | 212 | 301 |
| 6374 | 85 | 204 | 202 | 212 | 301 |
| 6375 | 94 | 201 | 212 | 212 | 304 |
| 6376 | 86 | 212 | 212 | 212 | 301 |
| 6377 | 68 | 204 | 212 | 212 | 304 |
| 6378 | 58 | 202 | 212 | 212 | 306 |
| 6379 | 54 | 212 | 201 | 201 | 302 |
| 6380 | 109 | 201 | 212 | 212 | 301 |
| 6381 | 9 | 212 | 212 | 212 | 305 |
| 6382 | 66 | 201 | 212 | 212 | 304 |
| 6383 | 96 | 201 | 201 | 201 | 308 |
| 6384 | 86 | 201 | 212 | 212 | 302 |
| 6385 | 101 | 201 | 212 | 212 | 308 |
| 6386 | 84 | 203 | 212 | 212 | 303 |
| 6387 | 96 | 212 | 201 | 201 | 301 |
| 6388 | 109 | 207 | 201 | 201 | 302 |
| 6389 | 85 | 210 | 202 | 212 | 303 |
| 6390 | 20 | 201 | 201 | 201 | 305 |
| 6391 | 57 | 202 | 212 | 212 | 308 |
| 6392 | 60 | 202 | 201 | 201 | 301 |
| 6393 | 113 | 210 | 201 | 201 | 307 |
| 6394 | 109 | 207 | 201 | 201 | 304 |
| 6395 | 22 | 211 | 201 | 201 | 308 |
| 6396 | 89 | 206 | 202 | 212 | 306 |
| 6397 | 101 | 212 | 202 | 212 | 307 |
| 6398 | 109 | 207 | 201 | 201 | 306 |
| 6399 | 91 | 210 | 201 | 201 | 308 |
| 6400 | 91 | 212 | 201 | 201 | 304 |
| 6401 | 37 | 204 | 202 | 212 | 306 |
| 6402 | 114 | 212 | 202 | 212 | 304 |
| 6403 | 80 | 204 | 212 | 212 | 306 |
| 6404 | 98 | 212 | 201 | 201 | 307 |
| 6405 | 46 | 204 | 202 | 212 | 308 |
| 6406 | 98 | 209 | 201 | 201 | 307 |
| 6407 | 73 | 210 | 202 | 212 | 308 |
| 6408 | 28 | 212 | 202 | 212 | 302 |
| 6409 | 129 | 211 | 202 | 212 | 302 |
| 6410 | 19 | 202 | 201 | 201 | 308 |
| 6411 | 79 | 206 | 212 | 212 | 301 |
| 6412 | 83 | 211 | 212 | 212 | 303 |
| 6413 | 103 | 205 | 201 | 201 | 301 |
| 6414 | 86 | 210 | 212 | 212 | 303 |
| 6415 | 64 | 212 | 201 | 201 | 308 |
| 6416 | 65 | 212 | 212 | 212 | 306 |
| 6417 | 96 | 210 | 212 | 212 | 305 |
| 6418 | 8 | 204 | 212 | 212 | 301 |
| 6419 | 38 | 206 | 201 | 201 | 305 |
| 6420 | 72 | 204 | 202 | 212 | 305 |
| 6421 | 59 | 206 | 201 | 201 | 306 |
| 6422 | 29 | 210 | 202 | 212 | 306 |
| 6423 | 130 | 206 | 202 | 212 | 306 |
| 6424 | 104 | 210 | 202 | 212 | 307 |
| 6425 | 25 | 204 | 202 | 212 | 306 |
| 6426 | 98 | 204 | 212 | 212 | 301 |
| 6427 | 115 | 206 | 201 | 201 | 308 |
| 6428 | 44 | 202 | 201 | 201 | 307 |
| 6429 | 55 | 204 | 201 | 201 | 302 |
| 6430 | 81 | 210 | 212 | 212 | 301 |
| 6431 | 47 | 203 | 202 | 212 | 306 |
| 6432 | 88 | 204 | 202 | 212 | 306 |
| 6433 | 88 | 206 | 202 | 212 | 303 |
| 6434 | 96 | 204 | 201 | 201 | 308 |
| 6435 | 116 | 205 | 202 | 212 | 301 |
| 6436 | 90 | 205 | 212 | 212 | 305 |
| 6437 | 24 | 204 | 201 | 201 | 301 |
| 6438 | 76 | 202 | 202 | 212 | 306 |
| 6439 | 121 | 209 | 202 | 212 | 308 |
| 6440 | 93 | 205 | 212 | 212 | 307 |
| 6441 | 101 | 203 | 201 | 201 | 301 |
| 6442 | 91 | 203 | 201 | 201 | 306 |
| 6443 | 101 | 205 | 202 | 212 | 302 |
| 6444 | 32 | 211 | 212 | 212 | 301 |
| 6445 | 6 | 210 | 202 | 212 | 306 |
| 6446 | 109 | 204 | 201 | 201 | 303 |
| 6447 | 12 | 207 | 201 | 201 | 303 |
| 6448 | 101 | 207 | 212 | 212 | 301 |
| 6449 | 86 | 205 | 201 | 201 | 303 |
| 6450 | 18 | 202 | 202 | 212 | 306 |
| 6451 | 96 | 212 | 212 | 212 | 307 |
| 6452 | 82 | 209 | 202 | 212 | 302 |
| 6453 | 85 | 204 | 212 | 212 | 308 |
| 6454 | 127 | 210 | 201 | 201 | 301 |
| 6455 | 88 | 206 | 212 | 212 | 301 |
| 6456 | 63 | 210 | 202 | 212 | 308 |
| 6457 | 16 | 205 | 201 | 201 | 306 |
| 6458 | 11 | 212 | 212 | 212 | 306 |
| 6459 | 99 | 203 | 202 | 212 | 307 |
| 6460 | 31 | 208 | 212 | 212 | 305 |
| 6461 | 98 | 205 | 201 | 201 | 302 |
| 6462 | 3 | 203 | 202 | 212 | 302 |
| 6463 | 10 | 211 | 202 | 212 | 308 |
| 6464 | 87 | 206 | 202 | 212 | 302 |
| 6465 | 35 | 209 | 202 | 212 | 301 |
| 6466 | 26 | 209 | 212 | 212 | 303 |
| 6467 | 74 | 210 | 201 | 201 | 303 |
| 6468 | 36 | 212 | 201 | 201 | 301 |
| 6469 | 56 | 209 | 202 | 212 | 304 |
| 6470 | 88 | 209 | 212 | 212 | 307 |
| 6471 | 95 | 204 | 201 | 201 | 308 |
| 6472 | 96 | 209 | 201 | 201 | 302 |
| 6473 | 61 | 202 | 202 | 212 | 301 |
| 6474 | 77 | 204 | 212 | 212 | 301 |
| 6475 | 27 | 212 | 202 | 212 | 306 |
| 6476 | 105 | 212 | 202 | 212 | 306 |
| 6477 | 69 | 210 | 201 | 201 | 303 |
| 6478 | 41 | 202 | 202 | 212 | 302 |
| 6479 | 4 | 201 | 201 | 201 | 304 |
| 6480 | 7 | 204 | 201 | 201 | 307 |
| 6481 | 86 | 204 | 212 | 212 | 304 |
| 6482 | 23 | 205 | 202 | 212 | 301 |
| 6483 | 118 | 203 | 212 | 212 | 306 |
| 6484 | 101 | 211 | 201 | 201 | 306 |
| 6485 | 62 | 205 | 212 | 212 | 303 |
| 6486 | 78 | 204 | 201 | 201 | 307 |
| 6487 | 1 | 201 | 212 | 212 | 308 |
| 6488 | 70 | 205 | 201 | 201 | 302 |
| 6489 | 50 | 212 | 202 | 212 | 307 |
| 6490 | 91 | 206 | 212 | 212 | 307 |
| 6491 | 51 | 211 | 201 | 201 | 307 |
| 6492 | 125 | 201 | 212 | 212 | 308 |
| 6493 | 102 | 201 | 201 | 201 | 308 |
| 6494 | 45 | 205 | 201 | 201 | 303 |
| 6495 | 88 | 210 | 202 | 212 | 306 |
| 6496 | 88 | 210 | 201 | 201 | 301 |
| 6497 | 85 | 211 | 212 | 212 | 301 |
| 6498 | 15 | 211 | 212 | 212 | 302 |
| 6499 | 67 | 211 | 201 | 201 | 302 |
| 6500 | 40 | 210 | 202 | 212 | 306 |
| 6501 | 30 | 210 | 201 | 201 | 305 |
| 6502 | 53 | 204 | 201 | 201 | 303 |
| 6503 | 48 | 212 | 202 | 212 | 303 |
| 6504 | 122 | 201 | 201 | 201 | 308 |
| 6505 | 123 | 201 | 201 | 201 | 307 |
| 6506 | 43 | 212 | 202 | 212 | 308 |
| 6507 | 109 | 212 | 201 | 201 | 305 |
| 6508 | 13 | 204 | 201 | 201 | 302 |
| 6509 | 117 | 211 | 202 | 212 | 302 |
| 6510 | 5 | 205 | 201 | 201 | 302 |
| 6511 | 124 | 203 | 201 | 201 | 308 |
| 6512 | 98 | 208 | 201 | 201 | 308 |
| 6513 | 109 | 204 | 212 | 212 | 303 |
| 6514 | 52 | 201 | 202 | 212 | 306 |
| 6515 | 42 | 205 | 212 | 212 | 308 |
| 6516 | 126 | 212 | 202 | 212 | 307 |
| 6517 | 119 | 212 | 212 | 212 | 302 |
| 6518 | 49 | 209 | 201 | 201 | 307 |
| 6519 | 100 | 212 | 212 | 212 | 308 |
| 6520 | 21 | 212 | 202 | 212 | 301 |
| 6521 | 91 | 201 | 201 | 201 | 305 |
| 6522 | 2 | 201 | 201 | 201 | 308 |
| 6523 | 71 | 211 | 202 | 212 | 306 |
| 6524 | 110 | 207 | 201 | 201 | 302 |
| 6525 | 85 | 204 | 212 | 212 | 301 |

Compounds having the formula:

TABLE 38

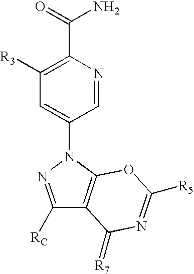

wherein R₃, R_{C}, R₅, and R₇ are defined in Table 38:

| Compound | | | | |
|---|---|---|---|---|
| No. | R₃ | R_{C} | R₅ | R₇ |
| 6526 | 65 | 212 | 212 | 306 |
| 6527 | 71 | 211 | 202 | 306 |
| 6528 | 88 | 210 | 201 | 301 |
| 6529 | 33 | 203 | 202 | 302 |
| 6530 | 127 | 210 | 201 | 301 |
| 6531 | 64 | 212 | 201 | 308 |
| 6532 | 123 | 201 | 201 | 307 |
| 6533 | 88 | 206 | 202 | 303 |
| 6534 | 27 | 212 | 202 | 306 |
| 6535 | 112 | 212 | 201 | 304 |
| 6536 | 75 | 204 | 202 | 302 |
| 6537 | 96 | 209 | 201 | 302 |
| 6538 | 86 | 205 | 201 | 303 |
| 6539 | 101 | 201 | 212 | 308 |
| 6540 | 48 | 212 | 202 | 303 |
| 6541 | 3 | 203 | 202 | 302 |
| 6542 | 88 | 206 | 212 | 301 |
| 6543 | 50 | 212 | 202 | 307 |
| 6544 | 119 | 212 | 212 | 302 |
| 6545 | 88 | 209 | 212 | 307 |
| 6546 | 60 | 202 | 201 | 301 |
| 6547 | 78 | 204 | 201 | 307 |
| 6548 | 101 | 205 | 202 | 302 |
| 6549 | 7 | 204 | 201 | 307 |
| 6550 | 46 | 204 | 202 | 308 |
| 6551 | 109 | 207 | 201 | 304 |
| 6552 | 1 | 201 | 212 | 308 |
| 6553 | 66 | 201 | 212 | 304 |
| 6554 | 9 | 212 | 212 | 305 |
| 6555 | 39 | 204 | 212 | 302 |
| 6556 | 74 | 210 | 201 | 303 |
| 6557 | 98 | 208 | 201 | 301 |
| 6558 | 26 | 209 | 212 | 303 |
| 6559 | 129 | 211 | 202 | 302 |
| 6560 | 96 | 212 | 201 | 301 |
| 6561 | 40 | 210 | 202 | 306 |
| 6562 | 108 | 205 | 212 | 302 |
| 6563 | 109 | 201 | 212 | 301 |
| 6564 | 98 | 212 | 201 | 307 |
| 6565 | 85 | 204 | 212 | 301 |
| 6566 | 86 | 201 | 202 | 301 |
| 6567 | 11 | 212 | 212 | 306 |
| 6568 | 89 | 206 | 202 | 306 |
| 6569 | 51 | 211 | 201 | 307 |
| 6570 | 85 | 212 | 212 | 307 |
| 6571 | 49 | 209 | 201 | 307 |
| 6572 | 91 | 203 | 201 | 306 |
| 6573 | 91 | 209 | 212 | 302 |
| 6574 | 83 | 211 | 212 | 303 |
| 6575 | 68 | 204 | 212 | 304 |
| 6576 | 88 | 210 | 202 | 306 |
| 6577 | 111 | 204 | 202 | 308 |
| 6578 | 98 | 204 | 212 | 301 |
| 6579 | 104 | 210 | 202 | 307 |
| 6580 | 6 | 210 | 202 | 306 |
| 6581 | 22 | 211 | 201 | 308 |
| 6582 | 110 | 207 | 201 | 302 |
| 6583 | 70 | 205 | 201 | 302 |
| 6584 | 44 | 202 | 201 | 307 |
| 6585 | 99 | 203 | 202 | 307 |
| 6586 | 23 | 205 | 202 | 301 |
| 6587 | 15 | 211 | 212 | 302 |
| 6588 | 98 | 205 | 201 | 302 |
| 6589 | 121 | 209 | 202 | 308 |
| 6590 | 82 | 209 | 202 | 302 |
| 6591 | 126 | 212 | 202 | 307 |
| 6592 | 57 | 202 | 212 | 308 |
| 6593 | 93 | 205 | 212 | 307 |
| 6594 | 88 | 211 | 201 | 306 |
| 6595 | 95 | 204 | 201 | 308 |
| 6596 | 30 | 210 | 201 | 305 |
| 6597 | 88 | 208 | 212 | 301 |
| 6598 | 85 | 210 | 202 | 303 |
| 6599 | 91 | 201 | 201 | 305 |
| 6600 | 122 | 201 | 201 | 308 |
| 6601 | 35 | 209 | 202 | 301 |
| 6602 | 47 | 203 | 202 | 306 |
| 6603 | 107 | 204 | 201 | 302 |
| 6604 | 43 | 212 | 202 | 308 |
| 6605 | 61 | 202 | 202 | 301 |
| 6606 | 97 | 210 | 201 | 304 |
| 6607 | 41 | 202 | 202 | 302 |
| 6608 | 86 | 204 | 212 | 304 |
| 6609 | 88 | 204 | 202 | 306 |
| 6610 | 109 | 204 | 212 | 303 |
| 6611 | 91 | 206 | 212 | 307 |
| 6612 | 12 | 207 | 201 | 303 |
| 6613 | 36 | 212 | 201 | 301 |
| 6614 | 109 | 204 | 201 | 303 |
| 6615 | 16 | 205 | 201 | 306 |
| 6616 | 10 | 211 | 202 | 308 |
| 6617 | 37 | 204 | 202 | 306 |
| 6618 | 92 | 204 | 212 | 301 |
| 6619 | 117 | 211 | 202 | 302 |
| 6620 | 100 | 212 | 212 | 308 |
| 6621 | 101 | 207 | 212 | 301 |
| 6622 | 101 | 212 | 202 | 307 |
| 6623 | 98 | 208 | 201 | 308 |
| 6624 | 113 | 210 | 201 | 307 |
| 6625 | 2 | 201 | 201 | 308 |
| 6626 | 31 | 208 | 212 | 305 |
| 6627 | 32 | 211 | 212 | 301 |
| 6628 | 55 | 204 | 201 | 302 |
| 6629 | 96 | 211 | 201 | 301 |
| 6630 | 38 | 206 | 201 | 305 |
| 6631 | 109 | 207 | 201 | 302 |
| 6632 | 116 | 205 | 202 | 301 |
| 6633 | 85 | 211 | 212 | 301 |
| 6634 | 115 | 206 | 201 | 308 |
| 6635 | 52 | 201 | 202 | 306 |
| 6636 | 25 | 204 | 202 | 306 |
| 6637 | 86 | 210 | 212 | 303 |
| 6638 | 42 | 205 | 212 | 308 |
| 6639 | 102 | 201 | 201 | 308 |
| 6640 | 101 | 211 | 201 | 306 |
| 6641 | 109 | 212 | 201 | 305 |
| 6642 | 63 | 210 | 202 | 308 |
| 6643 | 81 | 210 | 212 | 301 |
| 6644 | 128 | 212 | 201 | 303 |
| 6645 | 79 | 206 | 212 | 301 |
| 6646 | 85 | 204 | 212 | 308 |
| 6647 | 85 | 204 | 202 | 301 |
| 6648 | 96 | 201 | 201 | 304 |
| 6649 | 103 | 205 | 201 | 301 |
| 6650 | 114 | 212 | 202 | 304 |
| 6651 | 56 | 209 | 202 | 304 |
| 6652 | 77 | 204 | 212 | 301 |
| 6653 | 24 | 204 | 201 | 301 |
| 6654 | 120 | 201 | 201 | 304 |
| 6655 | 109 | 207 | 201 | 306 |
| 6656 | 53 | 204 | 201 | 303 |
| 6657 | 84 | 203 | 212 | 303 |
| 6658 | 87 | 206 | 202 | 302 |
| 6659 | 96 | 212 | 212 | 307 |
| 6660 | 17 | 207 | 201 | 308 |
| 6661 | 130 | 206 | 202 | 306 |
| 6662 | 91 | 212 | 201 | 304 |
| 6663 | 62 | 205 | 212 | 303 |
| 6664 | 72 | 204 | 202 | 305 |
| 6665 | 67 | 211 | 201 | 302 |
| 6666 | 90 | 205 | 212 | 305 |
| 6667 | 80 | 204 | 212 | 306 |
| 6668 | 59 | 206 | 201 | 306 |
| 6669 | 58 | 202 | 212 | 306 |
| 6670 | 45 | 205 | 201 | 303 |
| 6671 | 73 | 210 | 202 | 308 |
| 6672 | 118 | 203 | 212 | 306 |
| 6673 | 8 | 204 | 212 | 301 |
| 6674 | 125 | 201 | 212 | 308 |
| 6675 | 96 | 201 | 201 | 308 |
| 6676 | 34 | 205 | 201 | 304 |
| 6677 | 106 | 205 | 212 | 307 |
| 6678 | 18 | 202 | 202 | 306 |
| 6679 | 13 | 204 | 201 | 302 |
| 6680 | 98 | 209 | 201 | 307 |
| 6681 | 54 | 212 | 201 | 302 |
| 6682 | 91 | 210 | 201 | 308 |
| 6683 | 28 | 212 | 202 | 302 |
| 6684 | 86 | 212 | 212 | 301 |
| 6685 | 96 | 210 | 212 | 305 |
| 6686 | 19 | 202 | 201 | 308 |
| 6687 | 105 | 212 | 202 | 306 |
| 6688 | 96 | 204 | 201 | 308 |
| 6689 | 124 | 203 | 201 | 308 |
| 6690 | 4 | 201 | 201 | 304 |
| 6691 | 76 | 202 | 202 | 306 |
| 6692 | 21 | 212 | 202 | 301 |
| 6693 | 5 | 205 | 201 | 302 |
| 6694 | 101 | 203 | 201 | 301 |
| 6695 | 20 | 201 | 201 | 305 |
| 6696 | 69 | 210 | 201 | 303 |
| 6697 | 29 | 210 | 202 | 306 |
| 6698 | 94 | 201 | 212 | 304 |
| 6699 | 14 | 203 | 201 | 308 |
| 6700 | 86 | 201 | 212 | 302 |

Compounds having the formula:

TABLE 39

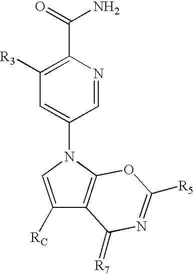

wherein R₃, R_{C}, R₅, and R₇ are defined in Table 39:

| Compound | | | | |
|---|---|---|---|---|
| No. | R₃ | R_{C} | R₅ | R₇ |
| 6701 | 32 | 211 | 212 | 301 |
| 6702 | 86 | 210 | 212 | 303 |
| 6703 | 86 | 204 | 212 | 304 |
| 6704 | 96 | 209 | 201 | 302 |
| 6705 | 27 | 212 | 202 | 306 |
| 6706 | 109 | 207 | 201 | 306 |
| 6707 | 67 | 211 | 201 | 302 |
| 6708 | 39 | 204 | 212 | 302 |
| 6709 | 96 | 201 | 201 | 304 |
| 6710 | 37 | 204 | 202 | 306 |
| 6711 | 54 | 212 | 201 | 302 |
| 6712 | 55 | 204 | 201 | 302 |
| 6713 | 85 | 204 | 202 | 301 |
| 6714 | 106 | 205 | 212 | 307 |
| 6715 | 96 | 204 | 201 | 308 |
| 6716 | 88 | 210 | 202 | 306 |
| 6717 | 70 | 205 | 201 | 302 |
| 6718 | 109 | 207 | 201 | 304 |
| 6719 | 6 | 210 | 202 | 306 |
| 6720 | 58 | 202 | 212 | 306 |
| 6721 | 105 | 212 | 202 | 306 |
| 6722 | 25 | 204 | 202 | 306 |
| 6723 | 101 | 201 | 212 | 308 |
| 6724 | 74 | 210 | 201 | 303 |
| 6725 | 116 | 205 | 202 | 301 |
| 6726 | 119 | 212 | 212 | 302 |
| 6727 | 104 | 210 | 202 | 307 |
| 6728 | 21 | 212 | 202 | 301 |
| 6729 | 93 | 205 | 212 | 307 |
| 6730 | 48 | 212 | 202 | 303 |
| 6731 | 96 | 212 | 201 | 301 |
| 6732 | 69 | 210 | 201 | 303 |
| 6733 | 117 | 211 | 202 | 302 |
| 6734 | 110 | 207 | 201 | 302 |
| 6735 | 78 | 204 | 201 | 307 |
| 6736 | 34 | 205 | 201 | 304 |
| 6737 | 53 | 204 | 201 | 303 |
| 6738 | 19 | 202 | 201 | 308 |
| 6739 | 2 | 201 | 201 | 308 |
| 6740 | 96 | 212 | 212 | 307 |
| 6741 | 107 | 204 | 201 | 302 |
| 6742 | 15 | 211 | 212 | 302 |
| 6743 | 85 | 204 | 212 | 301 |
| 6744 | 65 | 212 | 212 | 306 |
| 6745 | 101 | 212 | 202 | 307 |
| 6746 | 98 | 205 | 201 | 302 |
| 6747 | 88 | 208 | 212 | 301 |
| 6748 | 66 | 201 | 212 | 304 |
| 6749 | 97 | 210 | 201 | 304 |
| 6750 | 101 | 205 | 202 | 302 |
| 6751 | 79 | 206 | 212 | 301 |
| 6752 | 36 | 212 | 201 | 301 |
| 6753 | 42 | 205 | 212 | 308 |
| 6754 | 61 | 202 | 202 | 301 |
| 6755 | 109 | 201 | 212 | 301 |
| 6756 | 43 | 212 | 202 | 308 |
| 6757 | 118 | 203 | 212 | 306 |
| 6758 | 101 | 207 | 212 | 301 |
| 6759 | 85 | 211 | 212 | 301 |
| 6760 | 17 | 207 | 201 | 308 |
| 6761 | 35 | 209 | 202 | 301 |
| 6762 | 11 | 212 | 212 | 306 |
| 6763 | 108 | 205 | 212 | 302 |
| 6764 | 88 | 211 | 201 | 306 |
| 6765 | 98 | 209 | 201 | 307 |
| 6766 | 122 | 201 | 201 | 308 |
| 6767 | 125 | 201 | 212 | 308 |
| 6768 | 1 | 201 | 212 | 308 |
| 6769 | 51 | 211 | 201 | 307 |
| 6770 | 91 | 203 | 201 | 306 |
| 6771 | 90 | 205 | 212 | 305 |
| 6772 | 7 | 204 | 201 | 307 |
| 6773 | 86 | 205 | 201 | 303 |
| 6774 | 96 | 211 | 201 | 301 |
| 6775 | 41 | 202 | 202 | 302 |
| 6776 | 28 | 212 | 202 | 302 |
| 6777 | 86 | 201 | 212 | 302 |
| 6778 | 10 | 211 | 202 | 308 |
| 6779 | 16 | 205 | 201 | 306 |
| 6780 | 77 | 204 | 212 | 301 |
| 6781 | 52 | 201 | 202 | 306 |
| 6782 | 109 | 204 | 201 | 303 |
| 6783 | 94 | 201 | 212 | 304 |
| 6784 | 113 | 210 | 201 | 307 |
| 6785 | 75 | 204 | 202 | 302 |
| 6786 | 112 | 212 | 201 | 304 |
| 6787 | 84 | 203 | 212 | 303 |
| 6788 | 85 | 204 | 212 | 308 |
| 6789 | 56 | 209 | 202 | 304 |
| 6790 | 109 | 207 | 201 | 302 |
| 6791 | 124 | 203 | 201 | 308 |
| 6792 | 126 | 212 | 202 | 307 |
| 6793 | 8 | 204 | 212 | 301 |
| 6794 | 101 | 211 | 201 | 306 |
| 6795 | 88 | 210 | 201 | 301 |
| 6796 | 85 | 212 | 212 | 307 |
| 6797 | 64 | 212 | 201 | 308 |
| 6798 | 98 | 208 | 201 | 308 |
| 6799 | 38 | 206 | 201 | 305 |
| 6800 | 109 | 204 | 212 | 303 |
| 6801 | 80 | 204 | 212 | 306 |
| 6802 | 18 | 202 | 202 | 306 |
| 6803 | 60 | 202 | 201 | 301 |
| 6804 | 24 | 204 | 201 | 301 |
| 6805 | 76 | 202 | 202 | 306 |
| 6806 | 46 | 204 | 202 | 308 |
| 6807 | 33 | 203 | 202 | 302 |
| 6808 | 31 | 208 | 212 | 305 |
| 6809 | 87 | 206 | 202 | 302 |
| 6810 | 71 | 211 | 202 | 306 |
| 6811 | 115 | 206 | 201 | 308 |
| 6812 | 96 | 201 | 201 | 308 |
| 6813 | 98 | 204 | 212 | 301 |
| 6814 | 123 | 201 | 201 | 307 |
| 6815 | 101 | 203 | 201 | 301 |
| 6816 | 9 | 212 | 212 | 305 |
| 6817 | 91 | 201 | 201 | 305 |
| 6818 | 111 | 204 | 202 | 308 |
| 6819 | 72 | 204 | 202 | 305 |
| 6820 | 44 | 202 | 201 | 307 |
| 6821 | 128 | 212 | 201 | 303 |
| 6822 | 127 | 210 | 201 | 301 |
| 6823 | 91 | 206 | 212 | 307 |
| 6824 | 49 | 209 | 201 | 307 |
| 6825 | 30 | 210 | 201 | 305 |
| 6826 | 82 | 209 | 202 | 302 |
| 6827 | 130 | 206 | 202 | 306 |
| 6828 | 91 | 209 | 212 | 302 |
| 6829 | 63 | 210 | 202 | 308 |
| 6830 | 40 | 210 | 202 | 306 |
| 6831 | 29 | 210 | 202 | 306 |
| 6832 | 85 | 210 | 202 | 303 |
| 6833 | 73 | 210 | 202 | 308 |
| 6834 | 4 | 201 | 201 | 304 |
| 6835 | 86 | 212 | 212 | 301 |
| 6836 | 81 | 210 | 212 | 301 |
| 6837 | 98 | 212 | 201 | 307 |
| 6838 | 88 | 209 | 212 | 307 |
| 6839 | 57 | 202 | 212 | 308 |
| 6840 | 86 | 201 | 202 | 301 |
| 6841 | 20 | 201 | 201 | 305 |
| 6842 | 14 | 203 | 201 | 308 |
| 6843 | 68 | 204 | 212 | 304 |
| 6844 | 88 | 206 | 202 | 303 |
| 6845 | 99 | 203 | 202 | 307 |
| 6846 | 114 | 212 | 202 | 304 |
| 6847 | 47 | 203 | 202 | 306 |
| 6848 | 12 | 207 | 201 | 303 |
| 6849 | 62 | 205 | 212 | 303 |
| 6850 | 109 | 212 | 201 | 305 |
| 6851 | 89 | 206 | 202 | 306 |
| 6852 | 92 | 204 | 212 | 301 |
| 6853 | 45 | 205 | 201 | 303 |
| 6854 | 22 | 211 | 201 | 308 |
| 6855 | 59 | 206 | 201 | 306 |
| 6856 | 96 | 210 | 212 | 305 |
| 6857 | 120 | 201 | 201 | 304 |
| 6858 | 50 | 212 | 202 | 307 |
| 6859 | 103 | 205 | 201 | 301 |
| 6860 | 83 | 211 | 212 | 303 |
| 6861 | 13 | 204 | 201 | 302 |
| 6862 | 91 | 210 | 201 | 308 |
| 6863 | 91 | 212 | 201 | 304 |
| 6864 | 129 | 211 | 202 | 302 |
| 6865 | 5 | 205 | 201 | 302 |
| 6866 | 3 | 203 | 202 | 302 |
| 6867 | 102 | 201 | 201 | 308 |
| 6868 | 26 | 209 | 212 | 303 |
| 6869 | 88 | 206 | 212 | 301 |
| 6870 | 23 | 205 | 202 | 301 |
| 6871 | 88 | 204 | 202 | 306 |
| 6872 | 121 | 209 | 202 | 308 |
| 6873 | 98 | 208 | 201 | 301 |
| 6874 | 100 | 212 | 212 | 308 |

Compounds having the formula:

TABLE 40

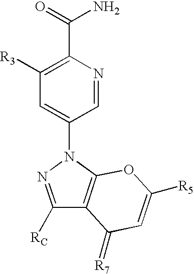

wherein R₃, R_{C}, R₅, and R₇ are defined in Table 40:

| Compound | | | | |
|---|---|---|---|---|
| No. | R₃ | R_{C} | R₅ | R₇ |
| 6875 | 88 | 209 | 212 | 307 |
| 6876 | 55 | 204 | 201 | 302 |
| 6877 | 88 | 210 | 201 | 301 |
| 6878 | 54 | 212 | 201 | 302 |
| 6879 | 17 | 207 | 201 | 308 |
| 6880 | 109 | 204 | 201 | 303 |
| 6881 | 61 | 202 | 202 | 301 |
| 6882 | 88 | 208 | 212 | 301 |
| 6883 | 130 | 206 | 202 | 306 |
| 6884 | 128 | 212 | 201 | 303 |
| 6885 | 101 | 203 | 201 | 301 |
| 6886 | 105 | 212 | 202 | 306 |
| 6887 | 52 | 201 | 202 | 306 |
| 6888 | 46 | 204 | 202 | 308 |
| 6889 | 15 | 211 | 212 | 302 |
| 6890 | 109 | 207 | 201 | 302 |
| 6891 | 63 | 210 | 202 | 308 |
| 6892 | 82 | 209 | 202 | 302 |
| 6893 | 85 | 210 | 202 | 303 |
| 6894 | 80 | 204 | 212 | 306 |
| 6895 | 124 | 203 | 201 | 308 |
| 6896 | 29 | 210 | 202 | 306 |
| 6897 | 7 | 204 | 201 | 307 |
| 6898 | 88 | 211 | 201 | 306 |
| 6899 | 27 | 212 | 202 | 306 |
| 6900 | 73 | 210 | 202 | 308 |
| 6901 | 86 | 205 | 201 | 303 |
| 6902 | 106 | 205 | 212 | 307 |
| 6903 | 83 | 211 | 212 | 303 |
| 6904 | 86 | 212 | 212 | 301 |
| 6905 | 68 | 204 | 212 | 304 |
| 6906 | 109 | 201 | 212 | 301 |
| 6907 | 12 | 207 | 201 | 303 |
| 6908 | 79 | 206 | 212 | 301 |
| 6909 | 30 | 210 | 201 | 305 |
| 6910 | 96 | 212 | 201 | 301 |
| 6911 | 71 | 211 | 202 | 306 |
| 6912 | 70 | 205 | 201 | 302 |
| 6913 | 88 | 206 | 202 | 303 |
| 6914 | 44 | 202 | 201 | 307 |
| 6915 | 11 | 212 | 212 | 306 |
| 6916 | 121 | 209 | 202 | 308 |
| 6917 | 96 | 209 | 201 | 302 |
| 6918 | 53 | 204 | 201 | 303 |
| 6919 | 4 | 201 | 201 | 304 |
| 6920 | 48 | 212 | 202 | 303 |
| 6921 | 94 | 201 | 212 | 304 |
| 6922 | 98 | 208 | 201 | 301 |
| 6923 | 9 | 212 | 212 | 305 |
| 6924 | 72 | 204 | 202 | 305 |
| 6925 | 109 | 204 | 212 | 303 |
| 6926 | 25 | 204 | 202 | 306 |
| 6927 | 85 | 211 | 212 | 301 |
| 6928 | 60 | 202 | 201 | 301 |
| 6929 | 76 | 202 | 202 | 306 |
| 6930 | 34 | 205 | 201 | 304 |
| 6931 | 91 | 212 | 201 | 304 |
| 6932 | 22 | 211 | 201 | 308 |
| 6933 | 24 | 204 | 201 | 301 |
| 6934 | 50 | 212 | 202 | 307 |
| 6935 | 91 | 210 | 201 | 308 |
| 6936 | 96 | 210 | 212 | 305 |
| 6937 | 88 | 210 | 202 | 306 |
| 6938 | 96 | 201 | 201 | 304 |
| 6939 | 56 | 209 | 202 | 304 |
| 6940 | 91 | 209 | 212 | 302 |
| 6941 | 74 | 210 | 201 | 303 |
| 6942 | 89 | 206 | 202 | 306 |
| 6943 | 85 | 204 | 202 | 301 |
| 6944 | 97 | 210 | 201 | 304 |
| 6945 | 117 | 211 | 202 | 302 |
| 6946 | 57 | 202 | 212 | 308 |
| 6947 | 45 | 205 | 201 | 303 |
| 6948 | 90 | 205 | 212 | 305 |
| 6949 | 100 | 212 | 212 | 308 |
| 6950 | 108 | 205 | 212 | 302 |
| 6951 | 125 | 201 | 212 | 308 |
| 6952 | 67 | 211 | 201 | 302 |
| 6953 | 14 | 203 | 201 | 308 |
| 6954 | 5 | 205 | 201 | 302 |
| 6955 | 1 | 201 | 212 | 308 |
| 6956 | 66 | 201 | 212 | 304 |
| 6957 | 98 | 209 | 201 | 307 |
| 6958 | 69 | 210 | 201 | 303 |
| 6959 | 113 | 210 | 201 | 307 |
| 6960 | 65 | 212 | 212 | 306 |
| 6961 | 85 | 204 | 212 | 308 |
| 6962 | 33 | 203 | 202 | 302 |
| 6963 | 20 | 201 | 201 | 305 |
| 6964 | 116 | 205 | 202 | 301 |
| 6965 | 41 | 202 | 202 | 302 |
| 6966 | 98 | 205 | 201 | 302 |
| 6967 | 101 | 211 | 201 | 306 |
| 6968 | 13 | 204 | 201 | 302 |
| 6969 | 78 | 204 | 201 | 307 |
| 6970 | 16 | 205 | 201 | 306 |
| 6971 | 8 | 204 | 212 | 301 |
| 6972 | 98 | 208 | 201 | 308 |
| 6973 | 93 | 205 | 212 | 307 |
| 6974 | 96 | 211 | 201 | 301 |
| 6975 | 107 | 204 | 201 | 302 |
| 6976 | 19 | 202 | 201 | 308 |
| 6977 | 35 | 209 | 202 | 301 |
| 6978 | 62 | 205 | 212 | 303 |
| 6979 | 120 | 201 | 201 | 304 |
| 6980 | 96 | 212 | 212 | 307 |
| 6981 | 47 | 203 | 202 | 306 |
| 6982 | 122 | 201 | 201 | 308 |
| 6983 | 64 | 212 | 201 | 308 |
| 6984 | 87 | 206 | 202 | 302 |
| 6985 | 104 | 210 | 202 | 307 |
| 6986 | 51 | 211 | 201 | 307 |
| 6987 | 127 | 210 | 201 | 301 |
| 6988 | 111 | 204 | 202 | 308 |
| 6989 | 85 | 204 | 212 | 301 |
| 6990 | 37 | 204 | 202 | 306 |
| 6991 | 91 | 206 | 212 | 307 |
| 6992 | 109 | 207 | 201 | 304 |
| 6993 | 85 | 212 | 212 | 307 |
| 6994 | 32 | 211 | 212 | 301 |
| 6995 | 21 | 212 | 202 | 301 |
| 6996 | 86 | 210 | 212 | 303 |
| 6997 | 10 | 211 | 202 | 308 |
| 6998 | 88 | 206 | 212 | 301 |
| 6999 | 95 | 204 | 201 | 308 |
| 7000 | 115 | 206 | 201 | 308 |
| 7001 | 75 | 204 | 202 | 302 |
| 7002 | 92 | 204 | 212 | 301 |
| 7003 | 31 | 208 | 212 | 305 |
| 7004 | 49 | 209 | 201 | 307 |
| 7005 | 109 | 207 | 201 | 306 |
| 7006 | 109 | 212 | 201 | 305 |
| 7007 | 101 | 205 | 202 | 302 |
| 7008 | 110 | 207 | 201 | 302 |
| 7009 | 101 | 207 | 212 | 301 |
| 7010 | 42 | 205 | 212 | 308 |
| 7011 | 101 | 201 | 212 | 308 |
| 7012 | 6 | 210 | 202 | 306 |
| 7013 | 98 | 212 | 201 | 307 |
| 7014 | 40 | 210 | 202 | 306 |
| 7015 | 88 | 204 | 202 | 306 |
| 7016 | 114 | 212 | 202 | 304 |
| 7017 | 91 | 201 | 201 | 305 |
| 7018 | 98 | 204 | 212 | 301 |
| 7019 | 3 | 203 | 202 | 302 |
| 7020 | 36 | 212 | 201 | 301 |
| 7021 | 58 | 202 | 212 | 306 |
| 7022 | 38 | 206 | 201 | 305 |
| 7023 | 102 | 201 | 201 | 308 |
| 7024 | 86 | 201 | 202 | 301 |
| 7025 | 101 | 212 | 202 | 307 |
| 7026 | 28 | 212 | 202 | 302 |
| 7027 | 96 | 204 | 201 | 308 |
| 7028 | 43 | 212 | 202 | 308 |
| 7029 | 103 | 205 | 201 | 301 |
| 7030 | 23 | 205 | 202 | 301 |
| 7031 | 112 | 212 | 201 | 304 |
| 7032 | 18 | 202 | 202 | 306 |
| 7033 | 81 | 210 | 212 | 301 |
| 7034 | 59 | 206 | 201 | 306 |
| 7035 | 119 | 212 | 212 | 302 |
| 7036 | 96 | 201 | 201 | 308 |
| 7037 | 2 | 201 | 201 | 308 |
| 7038 | 126 | 212 | 202 | 307 |
| 7039 | 86 | 201 | 212 | 302 |
| 7040 | 39 | 204 | 212 | 302 |
| 7041 | 77 | 204 | 212 | 301 |
| 7042 | 129 | 211 | 202 | 302 |
| 7043 | 26 | 209 | 212 | 303 |
| 7044 | 86 | 204 | 212 | 304 |
| 7045 | 84 | 203 | 212 | 303 |
| 7046 | 91 | 203 | 201 | 306 |
| 7047 | 118 | 203 | 212 | 306 |
| 7048 | 99 | 203 | 202 | 307 |
| 7049 | 123 | 201 | 201 | 307 |

Compounds having the formula:

TABLE 41

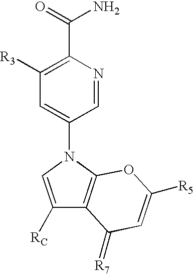

wherein R₃, R_{C}, R₅, and R₇ are defined in Table 41:

| Compound | | | | |
|---|---|---|---|---|
| No. | R₃ | R_{C} | R₅ | R₇ |
| 7050 | 9 | 212 | 212 | 305 |
| 7051 | 98 | 205 | 201 | 302 |
| 7052 | 89 | 206 | 202 | 306 |
| 7053 | 15 | 211 | 212 | 302 |
| 7054 | 91 | 210 | 201 | 308 |
| 7055 | 33 | 203 | 202 | 302 |
| 7056 | 126 | 212 | 202 | 307 |
| 7057 | 22 | 211 | 201 | 308 |
| 7058 | 109 | 207 | 201 | 304 |
| 7059 | 10 | 211 | 202 | 308 |
| 7060 | 100 | 212 | 212 | 308 |
| 7061 | 29 | 210 | 202 | 306 |
| 7062 | 30 | 210 | 201 | 305 |
| 7063 | 119 | 212 | 212 | 302 |
| 7064 | 109 | 212 | 201 | 305 |
| 7065 | 67 | 211 | 201 | 302 |
| 7066 | 42 | 205 | 212 | 308 |
| 7067 | 101 | 205 | 202 | 302 |
| 7068 | 32 | 211 | 212 | 301 |
| 7069 | 19 | 202 | 201 | 308 |
| 7070 | 68 | 204 | 212 | 304 |
| 7071 | 104 | 210 | 202 | 307 |
| 7072 | 96 | 212 | 201 | 301 |
| 7073 | 31 | 208 | 212 | 305 |
| 7074 | 88 | 210 | 201 | 301 |
| 7075 | 83 | 211 | 212 | 303 |
| 7076 | 7 | 204 | 201 | 307 |
| 7077 | 23 | 205 | 202 | 301 |
| 7078 | 63 | 210 | 202 | 308 |
| 7079 | 121 | 209 | 202 | 308 |
| 7080 | 86 | 201 | 202 | 301 |
| 7081 | 78 | 204 | 201 | 307 |
| 7082 | 77 | 204 | 212 | 301 |
| 7083 | 21 | 212 | 202 | 301 |
| 7084 | 115 | 206 | 201 | 308 |
| 7085 | 50 | 212 | 202 | 307 |
| 7086 | 27 | 212 | 202 | 306 |
| 7087 | 88 | 206 | 202 | 303 |
| 7088 | 96 | 204 | 201 | 308 |
| 7089 | 98 | 208 | 201 | 308 |
| 7090 | 4 | 201 | 201 | 304 |
| 7091 | 86 | 205 | 201 | 303 |
| 7092 | 17 | 207 | 201 | 308 |
| 7093 | 35 | 209 | 202 | 301 |
| 7094 | 90 | 205 | 212 | 305 |
| 7095 | 101 | 211 | 201 | 306 |
| 7096 | 69 | 210 | 201 | 303 |
| 7097 | 110 | 207 | 201 | 302 |
| 7098 | 84 | 203 | 212 | 303 |
| 7099 | 98 | 209 | 201 | 307 |
| 7100 | 118 | 203 | 212 | 306 |
| 7101 | 46 | 204 | 202 | 308 |
| 7102 | 111 | 204 | 202 | 308 |
| 7103 | 60 | 202 | 201 | 301 |
| 7104 | 25 | 204 | 202 | 306 |
| 7105 | 5 | 205 | 201 | 302 |
| 7106 | 101 | 212 | 202 | 307 |
| 7107 | 109 | 201 | 212 | 301 |
| 7108 | 71 | 211 | 202 | 306 |
| 7109 | 88 | 204 | 202 | 306 |
| 7110 | 13 | 204 | 201 | 302 |
| 7111 | 73 | 210 | 202 | 308 |
| 7112 | 99 | 203 | 202 | 307 |
| 7113 | 97 | 210 | 201 | 304 |
| 7114 | 92 | 204 | 212 | 301 |
| 7115 | 2 | 201 | 201 | 308 |
| 7116 | 96 | 210 | 212 | 305 |
| 7117 | 53 | 204 | 201 | 303 |
| 7118 | 41 | 202 | 202 | 302 |
| 7119 | 127 | 210 | 201 | 301 |
| 7120 | 76 | 202 | 202 | 306 |
| 7121 | 62 | 205 | 212 | 303 |
| 7122 | 43 | 212 | 202 | 308 |
| 7123 | 103 | 205 | 201 | 301 |
| 7124 | 16 | 205 | 201 | 306 |
| 7125 | 54 | 212 | 201 | 302 |
| 7126 | 58 | 202 | 212 | 306 |
| 7127 | 86 | 212 | 212 | 301 |
| 7128 | 91 | 201 | 201 | 305 |
| 7129 | 39 | 204 | 212 | 302 |
| 7130 | 36 | 212 | 201 | 301 |
| 7131 | 45 | 205 | 201 | 303 |
| 7132 | 130 | 206 | 202 | 306 |
| 7133 | 128 | 212 | 201 | 303 |
| 7134 | 117 | 211 | 202 | 302 |
| 7135 | 107 | 204 | 201 | 302 |
| 7136 | 93 | 205 | 212 | 307 |
| 7137 | 91 | 209 | 212 | 302 |
| 7138 | 114 | 212 | 202 | 304 |
| 7139 | 11 | 212 | 212 | 306 |
| 7140 | 108 | 205 | 212 | 302 |
| 7141 | 94 | 201 | 212 | 304 |
| 7142 | 85 | 204 | 212 | 301 |
| 7143 | 116 | 205 | 202 | 301 |
| 7144 | 52 | 201 | 202 | 306 |
| 7145 | 28 | 212 | 202 | 302 |
| 7146 | 105 | 212 | 202 | 306 |
| 7147 | 47 | 203 | 202 | 306 |
| 7148 | 101 | 207 | 212 | 301 |
| 7149 | 112 | 212 | 201 | 304 |
| 7150 | 38 | 206 | 201 | 305 |
| 7151 | 86 | 204 | 212 | 304 |
| 7152 | 12 | 207 | 201 | 303 |
| 7153 | 123 | 201 | 201 | 307 |
| 7154 | 18 | 202 | 202 | 306 |
| 7155 | 64 | 212 | 201 | 308 |
| 7156 | 95 | 204 | 201 | 308 |
| 7157 | 82 | 209 | 202 | 302 |
| 7158 | 109 | 204 | 212 | 303 |
| 7159 | 74 | 210 | 201 | 303 |
| 7160 | 85 | 211 | 212 | 301 |
| 7161 | 55 | 204 | 201 | 302 |
| 7162 | 66 | 201 | 212 | 304 |
| 7163 | 85 | 212 | 212 | 307 |
| 7164 | 91 | 203 | 201 | 306 |
| 7165 | 98 | 204 | 212 | 301 |
| 7166 | 88 | 206 | 212 | 301 |
| 7167 | 3 | 203 | 202 | 302 |
| 7168 | 8 | 204 | 212 | 301 |
| 7169 | 59 | 206 | 201 | 306 |
| 7170 | 96 | 201 | 201 | 304 |
| 7171 | 6 | 210 | 202 | 306 |
| 7172 | 14 | 203 | 201 | 308 |
| 7173 | 122 | 201 | 201 | 308 |
| 7174 | 96 | 212 | 212 | 307 |
| 7175 | 51 | 211 | 201 | 307 |
| 7176 | 85 | 210 | 202 | 303 |
| 7177 | 129 | 211 | 202 | 302 |
| 7178 | 85 | 204 | 202 | 301 |
| 7179 | 37 | 204 | 202 | 306 |
| 7180 | 87 | 206 | 202 | 302 |
| 7181 | 40 | 210 | 202 | 306 |
| 7182 | 20 | 201 | 201 | 305 |
| 7183 | 98 | 212 | 201 | 307 |
| 7184 | 88 | 208 | 212 | 301 |
| 7185 | 124 | 203 | 201 | 308 |
| 7186 | 56 | 209 | 202 | 304 |
| 7187 | 80 | 204 | 212 | 306 |
| 7188 | 86 | 210 | 212 | 303 |
| 7189 | 109 | 207 | 201 | 306 |
| 7190 | 113 | 210 | 201 | 307 |
| 7191 | 96 | 211 | 201 | 301 |
| 7192 | 88 | 211 | 201 | 306 |
| 7193 | 106 | 205 | 212 | 307 |
| 7194 | 65 | 212 | 212 | 306 |
| 7195 | 48 | 212 | 202 | 303 |
| 7196 | 125 | 201 | 212 | 308 |
| 7197 | 44 | 202 | 201 | 307 |
| 7198 | 85 | 204 | 212 | 308 |
| 7199 | 96 | 209 | 201 | 302 |
| 7200 | 88 | 209 | 212 | 307 |
| 7201 | 120 | 201 | 201 | 304 |
| 7202 | 102 | 201 | 201 | 308 |
| 7203 | 91 | 212 | 201 | 304 |
| 7204 | 101 | 203 | 201 | 301 |
| 7205 | 109 | 204 | 201 | 303 |
| 7206 | 86 | 201 | 212 | 302 |
| 7207 | 101 | 201 | 212 | 308 |
| 7208 | 91 | 206 | 212 | 307 |
| 7209 | 96 | 201 | 201 | 308 |
| 7210 | 24 | 204 | 201 | 301 |
| 7211 | 72 | 204 | 202 | 305 |
| 7212 | 75 | 204 | 202 | 302 |
| 7213 | 1 | 201 | 212 | 308 |
| 7214 | 49 | 209 | 201 | 307 |
| 7215 | 61 | 202 | 202 | 301 |
| 7216 | 109 | 207 | 201 | 302 |
| 7217 | 88 | 210 | 202 | 306 |
| 7218 | 26 | 209 | 212 | 303 |
| 7219 | 79 | 206 | 212 | 301 |
| 7220 | 81 | 210 | 212 | 301 |
| 7221 | 70 | 205 | 201 | 302 |
| 7222 | 57 | 202 | 212 | 308 |
| 7223 | 98 | 208 | 201 | 301 |
| 7224 | 34 | 205 | 201 | 304 |

### Biological Evaluation

### Example 33

### Cell Proliferation Assays

A panel of cancer cell lines is obtained from the DCTP Tumor Repository, National Cancer Institute (Frederick, Md.) or ATCC (Rockville, Md.). Cell cultures are maintained in Hyclone RPMI 1640 medium (Logan, Utah) supplemented with 10% fetal bovine serum and 20 mM HEPES buffer, final pH 7.2, at 37° C. with a 5% CO₂ atmosphere. Cultures are maintained at sub-confluent densities. Human umbilical vein endothelial cells (HUVEC) are purchased from Clonetics, a division of Cambrex (Walkersville, Md.). Cultures are established from cryopreserved stocks using Clonetics EGM-2 medium supplemented with 20 mM HEPES, final pH 7.2, at 37° C. with a 5% CO₂ atmosphere.

For proliferation assays, cells are seeded with the appropriate medium into 96 well plates at 1,000-2,500 cells per well, depending on the cell line, and are incubated overnight. The following day, test compound, DMSO solution (negative control), or Actinomycin D (positive control) is added to the appropriate wells as 10× concentrated stocks prepared in phosphate buffered saline. The cell plates are then incubated for an additional 2-5 days, depending on the cell line, to allow proliferation to occur. To measure cell density, 50 μL of WST-1 solution (Roche Applied Science, IN) diluted 1:5 in phosphate buffered saline is added to each well, and the cells incubated for an additional 1-5 hrs., again depending on the cell line. Optical density is determined for each well at 450 nM using a Tecan GeniosPro plate reader (RTP, NC). The percentage of cell growth is determined by comparing the cell growth in the presence of test compounds to the cells treated with DMSO vehicle (control, 100% growth) and cells treated with Actinomycin D (10 μM, 0% growth).

Immediately after the WST-1 determination, the medium is removed from the PC-3, NCI-H460 and HUVEC cell lines, and the plates stored at −80° C. Using these assay plates, relative amounts of DNA in each well are determined using the Cyquant DNA assay kit from R&D Systems (Eugene, Oreg.) following the manufacturer's directions. Results for each compound treatment are compared to DMSO vehicle control (100%) and 10 μM Actinomycin D treated cells (0%).

Compounds of this invention show inhibitory IC₅₀ values against these cell lines in the range of 0.01 μM to 50 μM.

### Example 34

### Determination of Affinity for HSP-90

### (Heat Shock Protein 90)

Affinity of test compounds for HSP-90 is determined as follows: Protein mixtures obtained from a variety of organ tissues (for example: spleen, liver and lung) are reversibly bound to a purine affinity column to capture purine-binding proteins, especially HSP-90. The purine affinity column is washed several times, and then eluted with 20 μM, 100 μM, and 500 μM of test compound. Compounds of Formula I elute HP-90 in a dose-dependent manner vs. a control elution using dimethylsulfoxide. The elution profile of Formula I compounds is determined by 1-dimensional SDS polyacrylamide gel electrophoresis. Gels are stained with a fluorescent stain such as sypro ruby (a highly sensitive fluorescent protein stain that can readily detect less than 1 fmol of total protein, i.e., less than 0.04 ng for a 40 kDa protein) or silver nitrate. The gels are imaged using a standard flat bed gel imager and the amount of protein estimated by densitometry. The percent of HSP-90 protein eluted from the column at each concentration is determined and IC₅₀ values are calculated from these estimates. Compounds of the invention are inhibitors of HSP-90 (heat shock protein 90) and have IC₅₀ values within the range of 0.01 μM to 50 μM.

Several exemplary compounds useful in the methods of the invention are listed below. The range of their relative binding affinity to HSP-90 is demonstrated, where +++ stands for very high, ++ for high and + for moderate.

| | | | |
|---|---|---|---|
| Compound 47 | ++ | Compound 51 | +++ |
| Compound 35 | +++ | Compound 34 | + |
| Compound 7 | +++ | Compound 27 | + |
| Compound 9 | + | Compound 3 | +++ |
| Compound 19 | +++ | Compound 21 | +++ |
| Compound 23 | ++ | Compound 46 | ++ |

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims. To particularly point out and distinctly claim the subject matter regarded as invention, the following claims conclude this specification.

What is claimed is:

1. A compound of the formula, 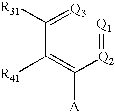
or a pharmaceutically acceptable salt thereof, wherein
each m is independently 0, 1, or 2;
each R_{C} independently is halogen, cyano, nitro, or —R_{N};
each R_{N} is independently —R_{N′}, —C(O)R_{N′}, —C(O)OR_{N′}, —C(O)N(R_{N′})₂, —S(O)R_{N′}, or —S(O)₂R_{N′} wherein
each R_{N′} is independently hydrogen, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl, wherein
each R_{N′} is optionally substituted with from 1 to 4 R groups;
each R_{O} is independently —R_{N′}, —C(O)R_{N′}, —C(O)OR_{N′}, or —C(O)N(R_{N′})₂;
each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆)alkylamino, carboxy, carboxamide, C₃-C₇ cycloalkyl, heterocycloalkyl, aryl, or heteroaryl;
Q₁, Q₂, and Q₃ are independently N or CR_{Q}, provided that no more than two of Q₁, Q₂, and Q₃ are not simultaneously N, wherein
each R_{Q} is independently hydrogen, halogen, —O(R_{O}), —N(R_{N})₂, C₁-C₆ alkyl, C₁-C₆ haloalkyl, C₃-C₇ cycloalkyl, aryl, or heteroaryl, or R₂₁, wherein
each R_{Q} is optionally substituted with from 1 to 4 R groups; and
R₂₁ is cyano, —C(O)OH, —C(O)—O(C₁-C₆alkyl), or —C(X)N(R₁₁₁)₂, wherein
each R₁₁₁ is independently H, hydroxy, C₁-C₆ alkyl, C₂-C₆ alkenyl, C₂-C₆ alkynyl, heteroaryl, aryl, C₃-C₈ cycloalkyl, heterocycloalkyl,
wherein each R₁₁₁ is optionally substituted with from 1 to 4 R groups;
or both R₁₁₁ taken together with the nitrogen to which they are attached, form a heterocycloalkyl; and
X is ═O, ═S, ═NH, ═NOH, ═N—NH₂, ═N—NH-aryl, ═N—═NH—(C₁-C₆ alkyl), or ═N—(C₁-C₆ alkoxy)
A is one of the formulas (i), (ii), (iii), or (iv), 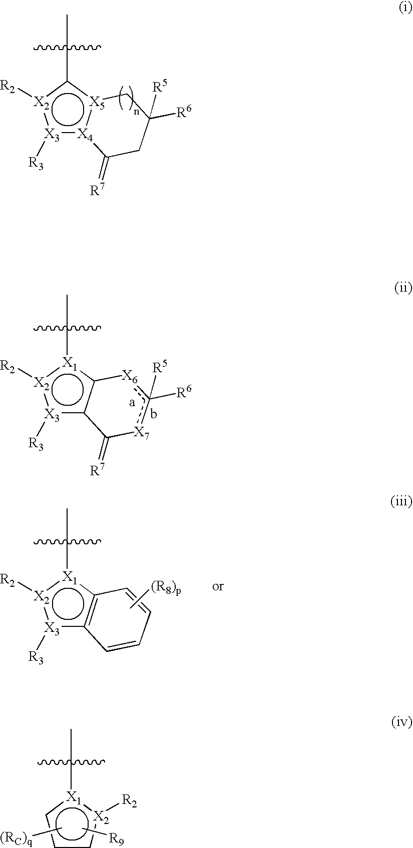
wherein
bonds a and b are independently a single or double bond, provided that
(i) when a is double bond, then b is a single bond, X₆ is CH or N, X₇ is CH₂ or NR_{N}, and R₆ is absent;
(ii) when b is double bond, then a is a single bond, X₆ is CH₂, O, S(O)ₘ, or NR_{N}, X₇ is CH or N, and R₆ is absent;
(iii) when a and b are both single bonds, then X₆ is O, S(O)ₘ, or NR_{N}, and X₇ is CH₂, NR_{N}, or O; and
n is 0, 1, 2, 3, or 4;
p is 1, 2, 3, or 4;
q is 0, 1, or 2;
X₁ is C or N;
X₂ and X₃ are independently C, N, O, or S;
X₄ and X₅ are independently C or N;
provided that
(i) for only formula (I),
(a) either exactly one of X₂, X₃, X₄, and X₅ is N, O, or S and the remaining three are C; or exactly two of X₂, X₃, X₄, and X₅ are N, and the remaining two are C; and
(b) X₄ and X₅ cannot be O or S;
(ii) for only formulas (II) and (iii),
(a) either exactly one of X₁, X₂, and X₃ is N, O, or S and the remaining two are C; or exactly two of X₁, X₂, and X₃ are N, and the remaining one is C; and
(b) X₁ cannot be S or O; and
(iii) for formula (iv), X₁ cannot be O or S;
provided that for all of formulas (i), (ii), (iii), and (iv) each of R₂ and R₃ is absent when the atom to which they are connected is of insufficient valency to carry a substituent;
R₂ is R_{C} when X₂ is C; or R₂ is R_{N} when X₂ is N;
R₃ is R_{C} when X₃ is C; or R₃ is R_{N} when X₃ is N;
R₅ and R₆ are independently H, C₁-C₆ alkyl, or aryl, wherein the aryl is optionally substituted with from 1 to 4 R groups,
wherein any two adjacent substituted aryl positions, together with the carbon atoms to which they are attached, optionally form an unsaturated cycloalkyl or heterocycloalkyl;
or R₅ and R₆ together with the carbon to which they are attached form a 3-8 membered ring;
R₇ is O, S, NH, N—OH, N—NH₂, N—NHR₂₂, N—NH—(C₁-C₆ alkyl), N—O— (CO—C₆)alkyl-R₂₂, or N—(C₁-C₆ alkoxy optionally substituted with carboxy);
each R₈ is independently —OR₈₁, —N(R₈₁)₂, or —R_{C},
wherein each R₈₁ is independently —H, —R₂₂, C₁-C₆ alkyl, or halo(C₁-C₆)alkyl,
wherein each R₈₁ is optionally substituted with 1-2 groups which are independently R_{C}, —OR_{O}, —SR_{O}, or —N(R_{N})₂;
R₉ is —C₁-C₆ alkoxy or a group of the formula, 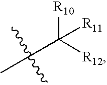
wherein
R₁₀ is C₁-C₆ alkyl or halo(C₁-C₆)alkyl, either optionally substituted with 1 or 2 R₁₀₁ groups, wherein
each R₁₀₁ is independently halogen, nitro, cyano, C₁-C₆ alkyl, halo(C₁-C₆)alkyl, —OR_{O}, —N(R_{N})₂, —S(O)ₘR_{N′}, or —C(O)R_{N′};
R₁₁ is H; and
R₁₂ is H or —OR_{O};
or R₁₁ and R₁₂ together are R₇; and
each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, wherein
each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
each R₂₂ is optionally fused to a C₆-C₁₀ aryl group, C₅-C₈ saturated cyclic group, or a C₅-C₁₀ heterocycloalkyl group;
or when A is formula (iv), R₉ and R_{C} together with the atoms to which they are attached optionally form a 5-7 membered carbocyclic ring fused to the ring carrying X₁ and X₂, the 5-7 membered ring being fused adjacent to X₂, and where the 5-7 membered carbocyclic ring is optionally substituted with oxo and 1-3 of C₁-C₆ alkyl; and
R₃₁ and R₄₁ are independently (a) H, (b) halo, or (c) a C₁-C₁₅ alkyl group where up to six of the carbon atoms in said alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein each (c) is optionally substituted with —R_{C}, —OR₁₅, —SR₁₅, —N(R₁₅)₂, or —R₂₂, wherein
each R₁₅ is independently —H, (C₁-C₁₀)alkyl, (C₁-C₁₀) haloalkyl, (C₂-C₆)alkenyl, (C₂-C₆)alkynyl, or (C₁-C₁₀)alkyl-Z, wherein
Z is —OR_{O} or —N(R₃₀)₂, wherein
each R₃₀ is independently —H or C₁-C₆ alkyl;
or N(R₃₀)₂ represents pyrrolidinyl, piperidinyl, piperazinyl, azepanyl, 1,3- or 1,4-diazepanyl, or morpholinyl, each of which is optionally substituted with R;
or R₃₁ and R₄₁ together with the atoms to which they are attached form a 5-12 membered mono-, bi-, or tricyclic ring system fused to the ring containing Q₁ and Q₂, where the 5-12 membered ring is partially unsaturated or aromatic and optionally contains one or two of oxygen, S(O)ₘ, nitrogen, or —NR₃₃ where R₃₃ is hydrogen or C₁-C₆ alkyl.

2. A compound according to claim 1, wherein
R₃₁ and R₄₁ are independently hydrogen, halo, or -Z₁R_{Z1}, wherein Z₁ is —O—, —NH—, —S(O)ₘ—, or —S(O)₂NH—, wherein
R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S(O)ₘ—(C₁-C₆)alkyl, —S(O)ₘ-aryl, —SO₂NH₂, —SO₂NH—(C₁-C₆)alkyl, —SO₂NH-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₂.

3. A compound according to claim 1, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂.

4. A compound according to claim 1, wherein
R₅ and R₆ are independently H, C₁-C₆ alkyl, or aryl.

5. A compound according to claim 1, wherein
R₅ and R₆ are independently H or C₁-C₆ alkyl.

6. A compound according to claim 1, wherein
R₇ is O or N—OH.

7. A compound according to claim 1, wherein
R₂₁ is cyano or —C(O)N(R₁₁₁)₂, wherein
each R₁₁₁ is independently H, hydroxy, C₁-C₆ alkyl, aryl, or C₃-C₈ cycloalkyl,
wherein each R₁₁₁ is optionally substituted with from 1 to 4 R groups.

8. A compound according to claim 1, wherein
R₂₁ is cyano or —C(O)NH₂.

9. A compound according to claim 1, of the formulae,

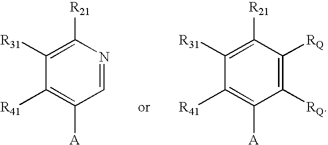

10. A compound according to claim 9, wherein A is one of the following,

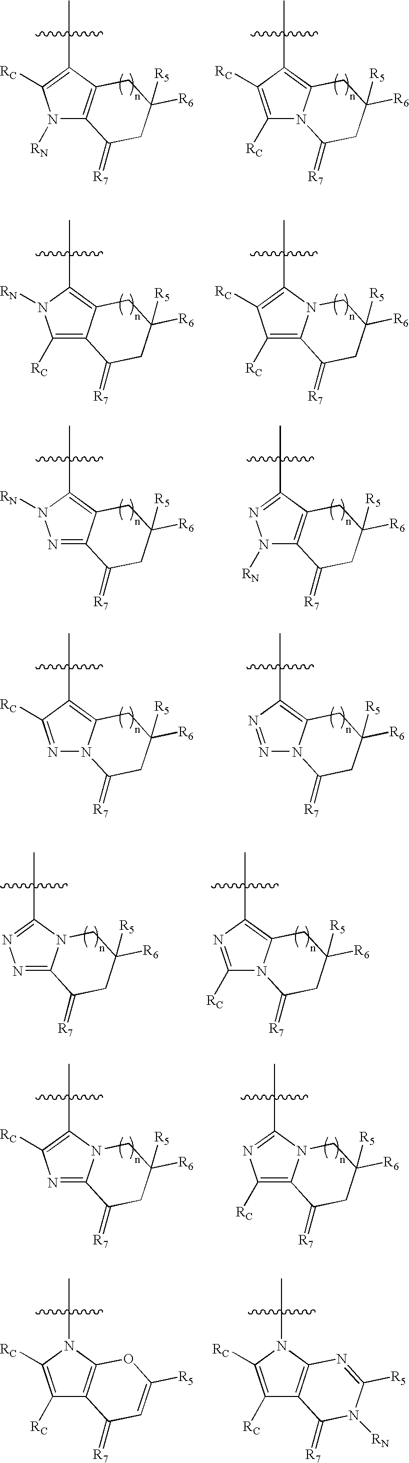

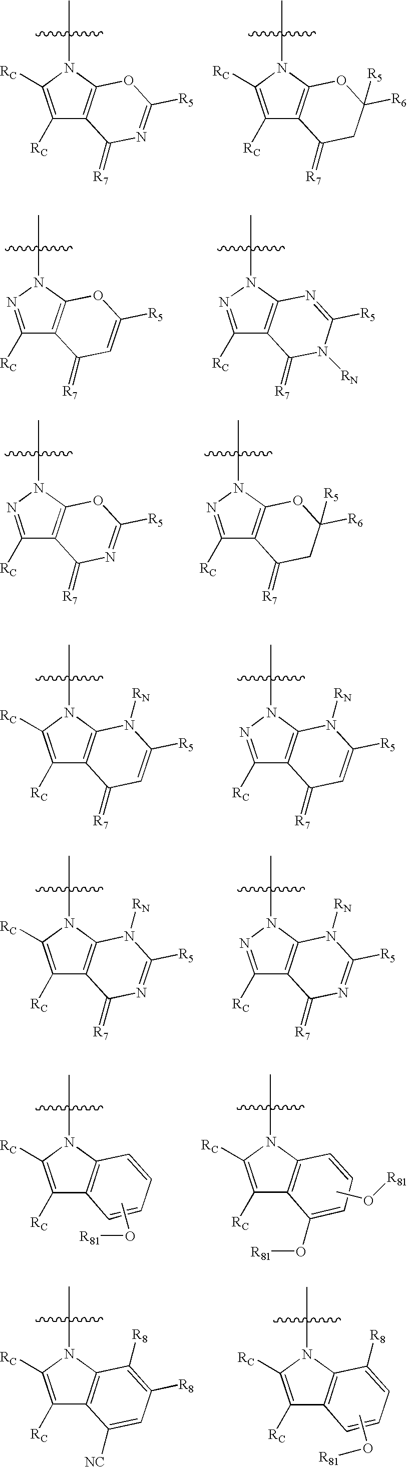

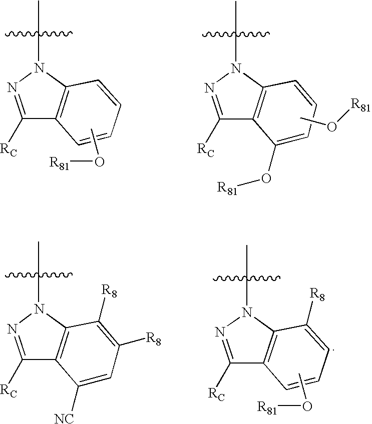

11. A compound according to claim 9, wherein A is one of the following,

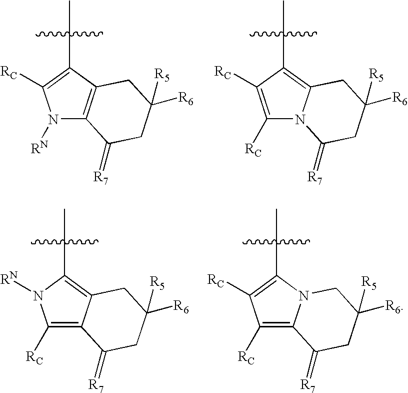

12. A compound according to claim 9, wherein A is one of the following,

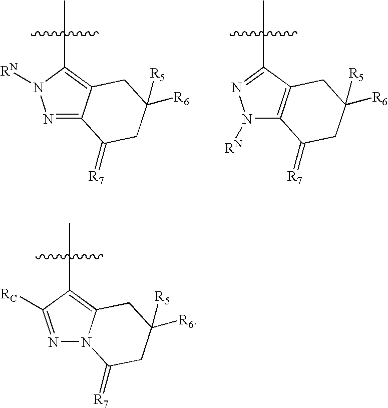

13. A compound according to claim 9, wherein A is one of the following,

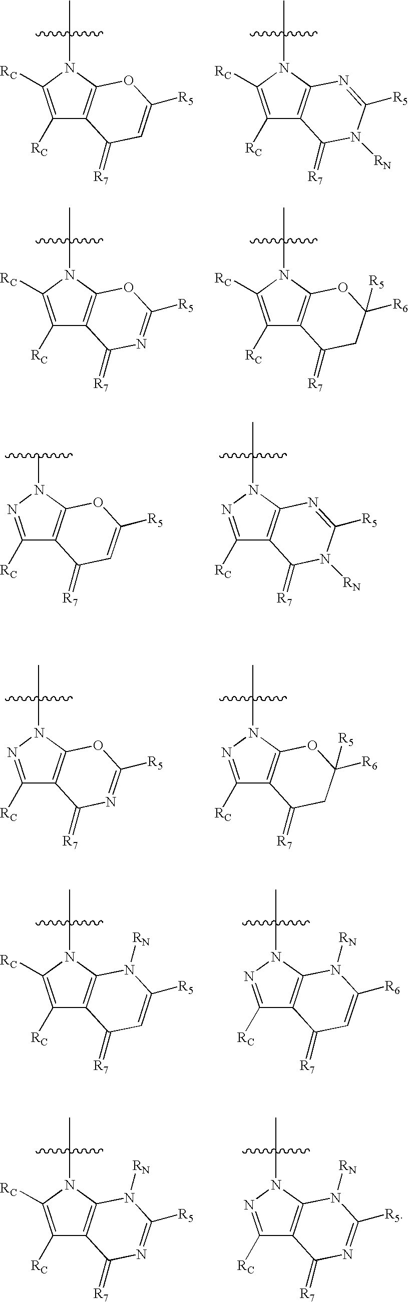

14. A compound according to claim 9, wherein A is one of the following,

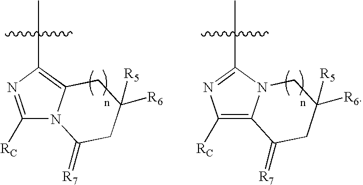

15. A compound according to claim 9, wherein A is one of the following,

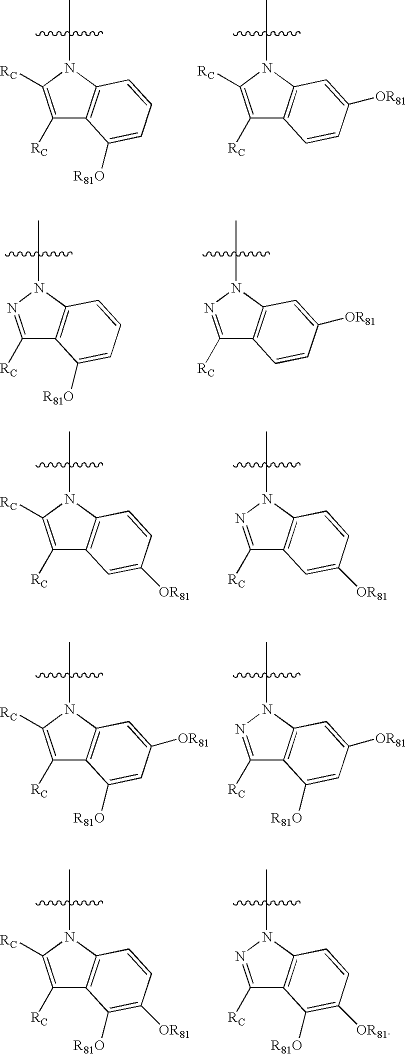

16. A compound according to claim 11, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂.

17. A compound according to claim 11, wherein
R₅ and R₆ are independently H, C₁-C₆ alkyl, or aryl.

18. A compound according to claim 11, wherein
R₇ is O or N—OH.

19. A compound according to claim 11, wherein
R₂₁ is —C(O)N(R₁₁₁)₂, wherein
each R₁₁₁ is independently H, hydroxy, C₁-C₆ alkyl, aryl, or C₃-C₈ cycloalkyl,
wherein each R₁₁₁ is optionally substituted with from 1 to 4 R groups.

20. A compound according to claim 11, wherein
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

21. A compound according to claim 11, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂;
R₅ and R₆ are independently H, C₁-C₆ alkyl, or aryl;
R₇ is O or N—OH;
R₂₁ is cyano or —C(O)N(R₁₁₁)₂,
each R₁₁₁ is independently H, hydroxy, C₁-C₆ alkyl, aryl, or C₃-C₈ cycloalkyl,
wherein each R₁₁₁ is optionally substituted with from 1 to 4 R groups; and
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

22. A compound according to claim 21, wherein
A is

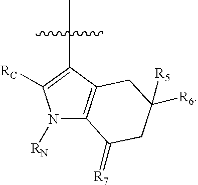

23. A compound according to claim 21, wherein
A is

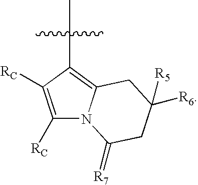

24. A compound according to claim 21, wherein
A is

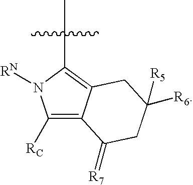

25. A compound according to claim 21, wherein
A is

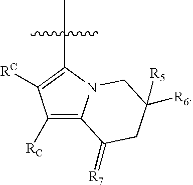

26. A compound according to claim 12, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂.

27. A compound according to claim 12, wherein
R₅ and R₆ are independently H, C₁-C₆ alkyl, or aryl.

28. A compound according to claim 12, wherein
R₇ is O or N—OH.

29. A compound according to claim 12, wherein
R₂₁ is —C(O)N(R₁₁₁)₂, wherein
each R₁₁₁ is independently H, hydroxy, C₁-C₆ alkyl, aryl, or C₃-C₈ cycloalkyl,
wherein each R₁₁₁ is optionally substituted with from 1 to 4 R groups.

30. A compound according to claim 12, wherein
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

31. A compound according to claim 12, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂;
R₅ and R₆ are independently H, C₁-C₆ alkyl, or aryl;
R₇ is O or N—OH;
R₂₁ is cyano or —C(O)N(R₁₁₁)₂, wherein
each R₁₁₁ is independently H, hydroxy, C₁-C₆ alkyl, aryl, or C₃-C₈ cycloalkyl,
wherein each R₁₁₁ is optionally substituted with from 1 to 4 R groups; and
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

32. A compound according to claim 31, wherein
A is

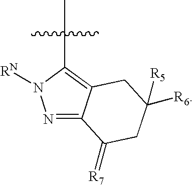

33. A compound according to claim 31, wherein
A is

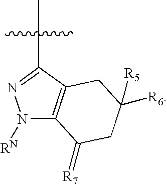

34. A compound according to claim 31, wherein
A is

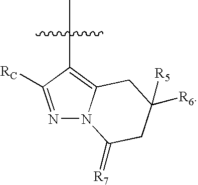

35. A compound according to claim 13, wherein
A is one of the following

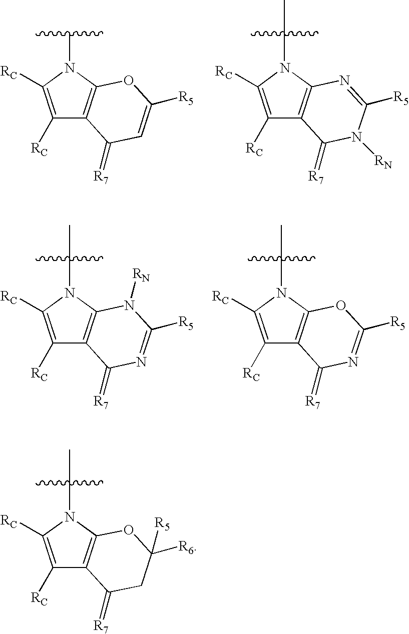

36. A compound according to claim 13, wherein
A is one of the following

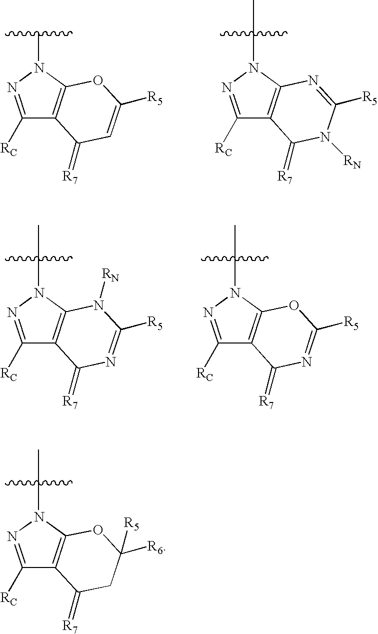

37. A compound according to claim 35, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂.

38. A compound according to claim 35, wherein
R₅ and R₆ are independently H, C₁-C₆ alkyl, or aryl.

39. A compound according to claim 35, wherein
R₇ is O or N—OH.

40. A compound according to claim 35, wherein
R₂₁ is —C(O)N(R₁₁₁)₂, wherein
each R₁₁₁ is independently H, hydroxy, C₁-C₆ alkyl, aryl, or C₃-C₈ cycloalkyl,
wherein each R₁₁₁ is optionally substituted with from 1 to 4 R groups.

41. A compound according to claim 35, wherein
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

42. A compound according to claim 35, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂;
R₅ and R₆ are independently H, C₁-C₆ alkyl, or aryl;
R₇ is O or N—OH;
R₂₁ is cyano or —C(O)N(R₁₁₁)₂, wherein
each R₁₁₁ is independently H, hydroxy, C₁-C₆ alkyl, aryl, or C₃-C₈ cycloalkyl,
wherein each R₁₁₁ is optionally substituted with from 1 to 4 R groups; and
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

43. A compound according to claim 42, wherein
A is

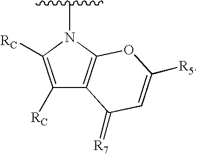

44. A compound according to claim 42, wherein
A is

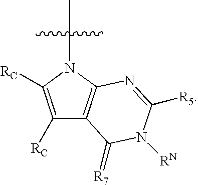

45. A compound according to claim 42, wherein
A is

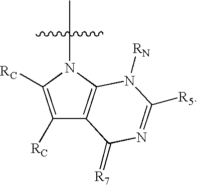

46. A compound according to claim 42, wherein
A is

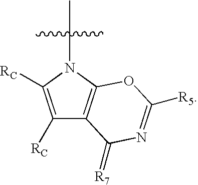

47. A compound according to claim 42, wherein
A is

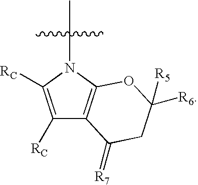

48. A compound according to claim 36, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂.

49. A compound according to claim 36, wherein
R₅ and R₆ are independently H, C₁-C₆ alkyl, or aryl.

50. A compound according to claim 36, wherein
R₇ is O or N—OH.

51. A compound according to claim 36, wherein
R₂₁ is —C(O)N(R₁₁₁)₂, wherein
each R₁₁₁ is independently H, hydroxy, C₁-C₆ alkyl, aryl, or C₃-C₈ cycloalkyl,
wherein each R₁₁₁ is optionally substituted with from 1 to 4 R groups.

52. A compound according to claim 36, wherein
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

53. A compound according to claim 36, wherein
R₃, and R₄, are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂;
R₅ and R₆ are independently H, C₁-C₆ alkyl, or aryl;
R₇ is O or N—OH;
R₂₁ is cyano or —C(O)N(R₁₁₁)₂, wherein
each R₁₁₁ is independently H, hydroxy, C₁-C₆ alkyl, aryl, or C₃-C₈ cycloalkyl,
wherein each R₁₁₁ is optionally substituted with from 1 to 4 R groups; and
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

54. A compound according to claim 53, wherein
A is

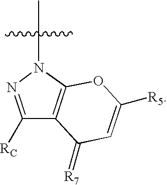

55. A compound according to claim 53, wherein
A is

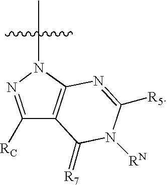

56. A compound according to claim 53, wherein
A is

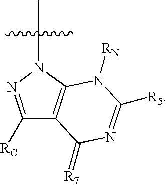

57. A compound according to claim 53, wherein
A is

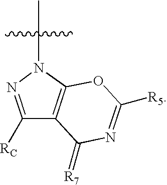

58. A compound according to claim 53, wherein
A is

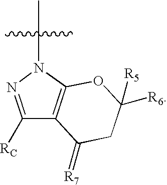

59. A compound according to claim 14, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂.

60. A compound according to claim 14, wherein
R₅ and R₆ are independently H, C₁-C₆ alkyl, or aryl.

61. A compound according to claim 14, wherein
R₇ is O or N—OH.

62. A compound according to claim 14, wherein
R₂₁ is —C(O)N(R₁₁₁)₂, wherein
each R₁₁₁ is independently y H, hydroxy, C₁-C₆ alkyl, aryl, or C₃-C₈ cycloalkyl,
wherein each R₁₁₁ is optionally substituted with from 1 to 4 R groups.

63. A compound according to claim 14, wherein
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

64. A compound according to claim 14, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂;
R₅ and R₆ are independently H, C₁-C₆ alkyl, or aryl;
R₇ is O or N—OH;
R₂₁ is cyano or —C(O)N(R₁₁₁)₂, wherein
each R₁₁₁ is independently H, hydroxy, C₁-C₆ alkyl, aryl, or C₃-C₈ cycloalkyl,
wherein each R₁₁₁ is optionally substituted with from 1 to 4 R groups; and
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

65. A compound according to claim 64, wherein
A is

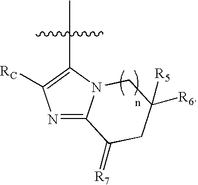

66. A compound according to claim 64, wherein
A is

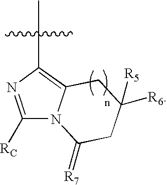

67. A compound according to claim 64, wherein
A is

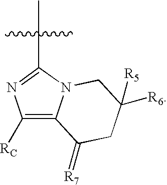

68. A compound according to claim 15, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂.

69. A compound according to claim 15, wherein
R₅ and R₆ are independently H, C₁-C₆ alkyl, or aryl.

70. A compound according to claim 15, wherein
R₇ is O or N—OH.

71. A compound according to claim 15, wherein
R₂₁ is —C(O)N(R₁₁₁)₂, wherein
each R₁₁₁ is independently H, hydroxy, C₁-C₆ alkyl, aryl, or C₃-C₈ cycloalkyl,
wherein each R₁₁₁ is optionally substituted with from 1 to 4 R groups.

72. A compound according to claim 15, wherein
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

73. A compound according to claim 15, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂;
R₅ and R₆ are independently H, C₁-C₆ alkyl, or aryl;
R₇ is O or N—OH;
R₂₁ is cyano or —C(O)N(R₁₁₁)₂, wherein
each R₁₁₁ is independently H, hydroxy, C₁-C₆ alkyl, aryl, or C₃-C₈ cycloalkyl,
wherein each R₁₁₁ is optionally substituted with from 1 to 4 R groups; and
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

74. A compound according to claim 73, wherein
A is

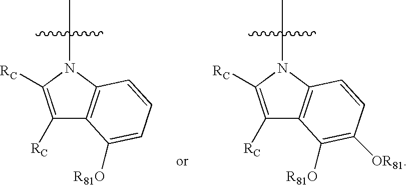

75. A compound according to claim 73, wherein
A is

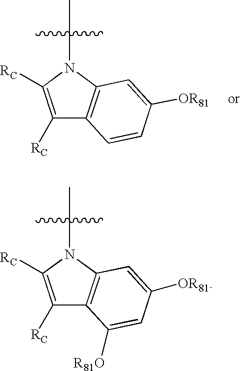

76. A compound according to claim 73, wherein
A is

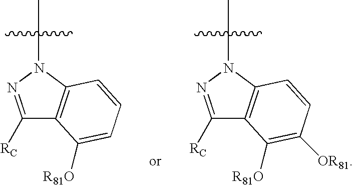

77. A compound according to claim 73, wherein
A is

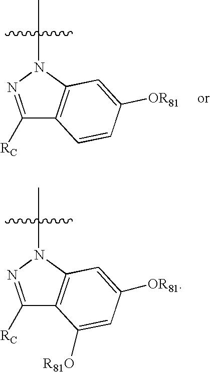

78. A compound according to claim 9, wherein A is one of the formulas, 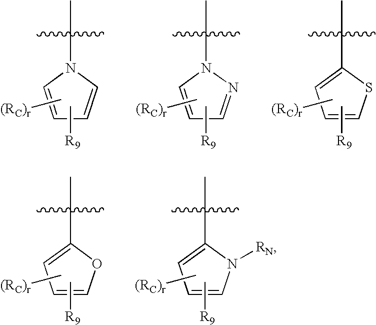
wherein r is 0 or 1.

79. A compound according to claim 78, wherein
R₉ is —CH(OH)—R₁₀,
wherein
R₁₀ is C₁-C₆ alkyl optionally substituted with 1 or 2 R₁₀₁ groups, wherein
each R₁₀₁ is independently halogen, nitro, cyano, C₁-C₆ alkyl, halo(C₁-C₆)alkyl, —OR_{O}, —N(R_{N})₂, —S(O)ₘR_{N′}, or —C(O)R_{N′}.

80. A compound according to claim 78, wherein
R₉ is —C(O)R₁₀,
wherein
R₁₀ is C₁-C₆ alkyl or halo(C₁-C₆)alkyl, either optionally substituted with 1 or 2 R₁₀₁ groups, wherein
each R₁₀₁ is independently halogen, nitro, cyano, C₁-C₆ alkyl, halo(C₁-C₆)alkyl, —OR_{O}, —N(R_{N})₂, —S(O)ₘR_{N′}, or —C(O)R_{N′}.

81. A compound according to claim 78, wherein
R₃₁ and R₄₁ are independently hydrogen, halo, or —N(H) R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂.

82. A compound according to claim 78, wherein
R₂₁ is —C(O)N(R₁₁₁)₂, wherein
each R₁₁₁ is independently H, hydroxy, C₁-C₆ alkyl, aryl, or C₃-C₈ cycloalkyl,
wherein each R₁₁₁ is optionally substituted with from 1 to 4 R groups.

83. A compound according to claim 78, wherein
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

84. A compound according to claim 78, wherein
R₃₁ and R₄₁ are independently hydrogen, halo, or —N(H)R_{Z1}, wherein R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, hydroxy, carboxy, carboxamido, oxo, halo, amino, C₁-C₆ alkoxy, mono- or di-(C₁-C₁₀)alkylamino, —OC₁-C₁₀ alkyl-Z, or R₂₂;
R₂₁ is cyano or —C(O)NH₂; and
each R_{N′} is independently hydrogen, C₁-C₁₀ alkyl, C₂-C₁₀ alkenyl, C₁-C₁₀ haloalkyl, C₃-C₇ cycloalkyl, C₃-C₇ cycloalkyl(C₁-C₁₀)alkyl.

85. A compound according to claim 84, wherein
A is

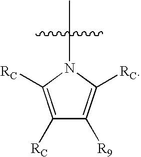

86. A compound according to claim 84, wherein
A is

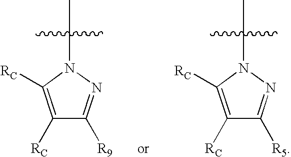

87. A compound according to claim 84, wherein
A is

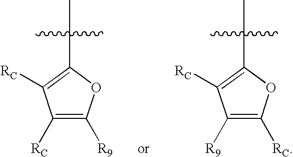

88. A compound according to claim 84, wherein
A is

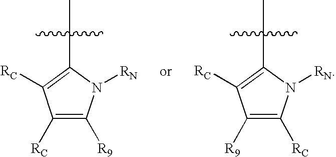

89. A compound according to claim 84, wherein
A is

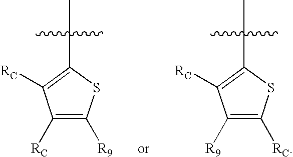

90. A compound according to claim 89, wherein
R₉ is —CH(OH)—R₁₀,
wherein
R₁₀ is C₁-C₆ alkyl optionally substituted with 1 or 2 R₁₀₁ groups, wherein
each R₁₀₁ is independently halogen, nitro, cyano, C₁-C₆ alkyl, halo(C₁-C₆)alkyl, —OR_{O}, —N(R_{N})₂, —S(O)ₘR_{N′}, or —C(O)R_{N′}.

91. A compound according to claim 89, wherein
R₉ is —C(O)R₁₀,
wherein
R₁₀ is C₁-C₆ alkyl or halo(C₁-C₆)alkyl, either optionally substituted with 1 or 2 R₁₀₁ groups, wherein
each R₁₀₁ is independently halogen, nitro, cyano, C₁-C₆ alkyl, halo(C₁-C₆)alkyl, —OR_{O}, —N(R_{N})₂, —S(O)ₘR_{N′}, or —C(O)R_{N′}.

92. A compound according to claim 1, which is
2-Bromo-4-(1,5,5-trimethyl-7-oxo-4,5,6,7-tetrahydro-1H-indazol-3-yl)-benzonitrile;
2-(Tetrahydro-pyran-4-ylamino)-4-(1,5,5-trimethyl-7-oxo-4,5,6,7-tetrahydro-1H-indazol-3-yl)-benzo-nitrile;
2-(Tetrahydro-pyran-4-ylamino)-4-(1,5,5-trimethyl-7-oxo-4,5,6,7-tetrahydro-1H-indazol-3-yl)-benzamide;
2-Bromo-4-(3,6,6-trimethyl-4-oxo-5,6-dihydro-4H-pyrano[2,3-c]pyrazol-1-yl)-benzonitrile;
2-Bromo-4-(3,6-dimethyl-4-oxo-4H-pyrano[2,3-c]pyrazol-1-yl)-benzonitrile;
4-(3,6-Dimethyl-4-oxo-4H-pyrano[2,3-c]pyrazol-1-yl)-2-(tetrahydro-pyran-4-ylamino)-benzonitrile;
4-(3,6-Dimethyl-4-oxo-4H-pyrano[2,3-c]pyrazol-1-yl)-2-(tetrahydro-pyran-4-ylamino)-benzamide;
4-(3-methyl-4-oxo-4,5-dihydro-1H-pyrazolo[3,4-d]pyrimidin-1-yl)-2-(tetrahydro-2H-pyran-4-ylamino)benzonitrile;
4-(3-Methyl-4-oxo-4,5-dihydro-pyrazolo[3,4-d]pyrimidin-1-yl)-2-(tetrahydro-pyran-4-ylamino)-benzamide;
4-(1-Benzyl-7-oxo-4,5,6,7-tetrahydro-1H-indol-3-yl)-benzonitrile;
4-(1-Benzyl-7-oxo-4,5,6,7-tetrahydro-1H-indol-3-yl)-benzamide;
4-[7-Oxo-1-(3,4,5-trimethoxy-benzyl)-4,5,6,7-tetrahydro-1H-indol-3-yl]-benzonitrile;
4-[7-Oxo-1-(3,4,5-trimethoxy-benzyl)-4,5,6,7-tetrahydro-1H-indol-3-yl]-benzamide;
2-Fluoro-4-(1,5,5-trimethyl-7-oxo-4,5,6,7-tetrahydro-1H-indol-3-yl)-benzonitrile;
2-[(Tetrahydro-furan-2-ylmethyl)-amino]-4-(1,5,5-trimethyl-7-oxo-4,5,6,7-tetrahydro-1H-indol-3-yl)-benzonitrile;
2-[(Tetrahydro-furan-2-ylmethyl)-amino]-4-(1,5,5-trimethyl-7-oxo-4,5,6,7-tetrahydro-1H-indol-3-yl-benzamide;
2-(4-Hydroxy-cyclohexylamino)-4-(1,5,5-trimethyl-7-oxo-4,5,6,7-tetrahydro-1H-indol-3-yl)-benzonitrile;
2-(4-Hydroxy-cyclohexylamino)-4-(1,5,5-trimethyl-7-oxo-4,5,6,7-tetrahydro-1H-indol-3-yl)-benzamide;
2-(4-Hydroxycyclohexylamino)-4-(6-methoxyindol-1-yl)benzonitrile;
2-(4-Hydroxycyclohexylamino)-4-(6-methoxyindol-1-yl)benzamide;
2-(4-Hydroxycyclohexylamino)-4-(4-methoxyindol-1-yl)benzonitrile;
2-(4-Hydroxycyclohexylamino)-4-(4-methoxyindol-1-yl)benzamide;
4-(5-Acetyl-thiophen-2-yl)-2-(4-hydroxy-cyclohexylamino)-benzonitrile;
4-(5-Acetyl-thiophen-2-yl)-2-(4-hydroxy-cyclohexylamino)-benzamide; or
pharmaceutically acceptable salts thereof.

93. A compound according to claim 1, which is
4-(1-Benzyl-7-oxo-4,5,6,7-tetrahydro-1H-indol-3-yl)-benzamide;
4-(3-Acetyl-2,4-dimethyl-pyrrol-1-yl)-2-bromo-benzonitrile;
4-(3-Acetyl-2,4-dimethyl-pyrrol-1-yl)-2-(2-methoxy-1-methyl-ethylamino)-benzonitrile;
4-(3-Acetyl-2,4-dimethyl-pyrrol-1-yl)-2-(2-methoxy-1-methyl-ethylamino)-benzamide;
4-(5-methoxy-2-methyl-1H-indol-1-yl)-2-(phenylamino)benzonitrile;
4-(5-methoxy-2-methyl-1H-indol-1-yl)-2-(phenylamino)benzamide;
2-fluoro-4-(8-oxo-5,6,7,8-tetrahydroindolizin-3-yl)benzonitrile;
2-(4-hydroxycyclohexylamino)-4-(8-oxo-5,6,7,8-tetrahydroindolizin-3-yl)benzamide; or
pharmaceutically acceptable salts thereof.

94. A compound according to claim 1, which is
4-(3-Methyl-4-oxo-4,5-dihydro-pyrazolo[3,4-d]pyrimidin-1-yl)-2-(tetrahydro-pyran-4-ylamino)-benzonitrile;
4-(1-Benzyl-7-oxo-4,5,6,7-tetrahydro-1H-indol-3-yl)-benzonitrile;
2-fluoro-4-(1-methyl-8-oxo-5,6,7,8-tetrahydroimidazo[1,5-a]pyridin-3-yl)benzonitrile;
4-(1-methyl-8-oxo-5,6,7,8-tetrahydroimidazo[1,5-a]pyridin-3-yl)-2-(tetrahydro-2H-pyran-4-ylamino)benzonitrile;
4-(1-methyl-8-oxo-5,6,7,8-tetrahydroimidazo[1,5-a]pyridin-3-yl)-2-(tetrahydro-2H-pyran-4-ylamino)benzamide; or
pharmaceutically acceptable salts thereof.

95. A compound according to claim 1, which is 4-(5-methoxy-2-methyl-1H-indol-1-yl)-2-(phenylamino)benzonitrile;
4-(5-methoxy-2-methyl-1H-indol-1-yl)-2-(phenylamino)benzamide;
4-(5,6-dimethoxy-1H-indol-1-yl)-2-(phenylamino)benzamide;
4-(5,6-dimethoxy-1H-indol-1-yl)-2-(phenylamino)benzonitrile;
5-amino-1-(4-carbamoyl-3-(tetrahydro-2H-pyran-4-ylamino)phenyl)-3-methyl-1H-pyrazole-4-carboxamide;
3-chloro-4-(5-methoxy-1H-indol-1-yl)benzamide;
3-chloro-4-(5-methoxy-1H-indol-1-yl)benzonitrile;
3-butoxy-4-(5,6-dimethoxy-1H-indol-1-yl)benzonitrile;
3-butoxy-4-(5,6-dimethoxy-1H-indol-1-yl)benzamide;
(Z)-3-butoxy-4-(5,6-dimethoxy-1H-indol-1-yl)-N′-hydroxybenzimidamide;
3-chloro-4-(5,6-dimethoxy-1H-indol-1-yl)benzonitrile;
3-chloro-4-(5,6-dimethoxy-1H-indol-1-yl)benzamide;
3-chloro-4-(1H-indol-1-yl)benzonitrile;
3-chloro-4-(1H-indol-1-yl)benzamide;
2-(2-methoxyethylamino)-4-(3,6,6-trimethyl-4-oxo-4,5,6,7-tetrahydro-2H-indazol-2-yl)benzamide;
4-(3-butyryl-2,5-dimethyl-1H-pyrrol-1-yl)benzamide;
4-(2,5-dimethyl-3-pivaloyl-1H-pyrrol-1-yl)benzamide;
2-(4-hydroxycyclohexylamino)-4-(8-oxo-5,6,7,8-tetrahydroindolizin-3-yl)benzonitrile;
4-(3-ethyl-6,6-dimethyl-4-oxo-4,5,6,7-tetrahydro-2H-indazol-2-yl)-2-((1S,2S)-2-hydroxycyclopentylamino)benzamide;
4-(3-acetyl-2,4-dimethyl-1H-pyrrol-1-yl)-2-bromobenzonitrile;
2-bromo-4-(2-ethyl-4-methyl-3-propionyl-1H-pyrrol-1-yl)benzonitrile;
4-(2-ethyl-4-methyl-3-propionyl-1H-pyrrol-1-yl)-2-(1-methoxypropan-2-ylamino)benzamide;
1-(4-carbamoylphenyl)-5-hydroxy-1H-pyrazole-4-carboxylic acid;
ethyl 1-(4-carbamoylphenyl)-5-hydroxy-1H-pyrazole-4-carboxylate; or
pharmaceutically acceptable salts thereof.

96. A pharmaceutical composition comprising at least one compound or salt according to claim 1 and a pharmaceutically acceptable solvent, carrier, excipient, adjuvant or a combination thereof.

97. A method of treating cancer, inflammation, or arthritis comprising administering to a patient in need of such treatment a therapeutically effective amount of a compound or salt of claim 1.

98. A method for treating a subject suffering from a disease or disorder of proteins that are either client proteins for HSP-90 or indirectly affect its client proteins, wherein disorder is selected from the group of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases, malignant disease, scleroderma, polymyositis, systemic lupus, rheumatoid arthritis, liver cirrhosis, keloid formation, interstitial nephritis, pulmonary fibrosis, and sepsis, comprising administering to a subject in need of such treatment a therapeutically effective amount of a compound or salt of claim 1.

99. A method of reducing the level of infection in a subject where the infection is caused by an organism selected from Plasmodium species, the method comprising administering to an infected subject an effective amount of a compound or salt according to claim 1.

100. A method for treating a fungal infection in a patient in need of such treatment, comprising administering an effective amount of a compound or salt according to claim 1 and an optional anti-fungal agent or drug.

101. A method according to claim 97, for the treatment of cancer and further comprising administration of (a) at least one additional anti-cancer agent or composition or (b) radiation therapy.

102. A method of treating a patient suffering from a viral infection comprising administering to the patient a therapeutically effective amount of a compound of claim 1.

103. A compound, which is
2-Bromo-4-(3-methoxy-5,5-dimethyl-2-oxo-cyclohex-3-enecarbonyl)-benzonitrile;
2-Bromo-4-(5-hydroxy-3-methyl-pyrazol-1-yl)-benzonitrile
2-Bromo-4-[5-hydroxy-3-methyl-4-(3-methyl-but-2-enoyl)-pyrazol-1-yl]-benzonitrile;
2-Bromo-4-{N′-[1-(4-hydroxy-6-methyl-2-oxo-2H-pyran-3-yl)-ethylidene]-hydrazino}-benzonitrile;
4-Hydrazino-2-(tetrahydro-pyran-4-ylamino)-benzonitrile;
4-Fluoro-2-(tetrahydro-pyran-4-ylamino)-benzonitrile;
5-Amino-1-[4-cyano-3-(tetrahydro-pyran-4-ylamino)-phenyl]-3-methyl-1H-pyrazole-4-carbonitrile;
2-Fluoro-4-(2-nitro-vinyl)-benzonitrile;
4-Bromo-2-(4-hydroxy-cyclohexylamino)-benzonitrile;
5-methyl-1-trityl-1H-imidazole-4-carbaldehyde;
1-(5-methyl-1-trityl-1H-imidazol-4-yl)-4-(tetrahydro-2H-pyran-2-yloxy)but-2-yn-1-ol;
1-(5-methyl-1-trityl-1H-imidazol-4-yl)-4-(tetrahydro-2H-pyran-2-yloxy)but-2-yn-1-one;
1-(5-methyl-1-trityl-1H-imidazol-4-yl)-4-(tetrahydro-2H-pyran-2-yloxy)butan-1-one;
4-hydroxy-1-(5-methyl-1-trityl-1H-imidazol-4-yl)butan-1-one;
1-methyl-6,7-dihydroimidazo[1,5-a]pyridin-8(5H)-one;
3-bromo-1-methyl-6,7-dihydroimidazo[1,5-a]pyridin-8(5H)-one;
4-(3-(1,3-dioxolan-2-yl)propanoyl)-2-fluorobenzonitrile;
4-(2,5-dimethoxytetrahydrofuran-2-yl)-2-fluorobenzonitrile;
4-(2-(4-cyano-3-fluorophenyl)-1H-pyrrol-1-yl)butanoic acid; or
pharmaceutically acceptable salts.

104. A process for preparing a compound of the formula F3 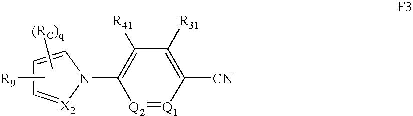
where
Q₁ and Q₂ are independently N or CR_{Q}, wherein
each R_{Q} is independently hydrogen, halogen, —O(R_{O}), —N(R_{N})₂, C₁-C₆ alkyl, C₁-C₆ haloalkyl, C₃-C₇ cycloalkyl, aryl, or heteroaryl, or R₂₁, wherein
each R_{Q} is optionally substituted with from 1 to 4 R groups; and
R₂₁ is cyano, —C(O)OH, —C(O)—O(C₁-C₆alkyl), or —C(X)N(R₁₁₁)₂, wherein
each R₁₁₁ is independently H, hydroxy, C₁-C₆ alkyl, C₂-C₆ alkenyl, C₂-C₆ alkynyl, heteroaryl, aryl, C₃-C₈ cycloalkyl, heterocycloalkyl,
wherein each R₁₁₁ is optionally substituted with from 1 to 4 R groups;
or both R₁₁₁ taken together with the nitrogen to which they are attached, form a heterocycloalkyl; and
X is ═O, ═S, ═NH, ═NOH, ═N—NH₂, ═N—NH-aryl, ═N—═NH—(C₁-C₆ alkyl), or ═N—(C₁-C₆ alkoxy)
wherein
m is 0, 1, or 2;
X₂ is C, N, O, or S;
R₉ is —C₁-C₆ alkoxy or a group of the formula, 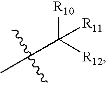
wherein
R₁₀ is C₁-C₆ alkyl or halo(C₁-C₆)alkyl, either optionally substituted with 1 or 2 R₁₀₁ groups, wherein
each R₁₀₁ is independently halogen, nitro, cyano, C₁-C₆ alkyl, halo(C₁-C₆)alkyl, —OR_{O}, —N(R_{N})₂, —S(O)ₘR_{N′}, or —C(O)R_{N′};
R₁₁ is H; and
R₁₂ is H or —OR_{O};
or R₁₁ and R₁₂ together are R₇; and
R₇ is O, S, NH, N—OH, N—NH₂, N—NHR₂₂, N—NH—(C₁-C₆ alkyl), N—O—(C₀-C₆)alkyl-R₂₂, or N—(C₁-C₆ alkoxy optionally substituted with carboxy);
each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, wherein
each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
each R₂₂ is optionally fused to a C₆-C₁₀ aryl group, C₅-C₈ saturated cyclic group, or a C₅-C₁₀ heterocycloalkyl group; and
R₃₁ and R₄₁ are independently (a) H, (b) halo, or (c) a C₁-C₁₅ alkyl group where up to six of the carbon atoms in said alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein each (c) is optionally substituted with —R_{C}, —OR₁₅, —SR₁₅, —N(R₁₅)₂, or —R₂₂, wherein
each R₁₅ is independently —H, (C₁-C₁₀)alkyl, (C₁-C₁₀) haloalkyl, (C₂-C₆)alkenyl, (C₂-C₆)alkynyl, or (C₁-C₁₀)alkyl-Z, wherein
Z is —OR_{O} or —N(R₃₀)₂, wherein
each R₃₀ is independently —H or C₁-C₆ alkyl;
or N(R₃₀)₂ represents pyrrolidinyl, piperidinyl, piperazinyl, azepanyl, 1,3- or 1,4-diazepanyl, or morpholinyl, each of which is optionally substituted with R;
or R₃₁ and R₄₁ together with the atoms to which they are attached form a 5-12 membered mono-, bi-, or tricyclic ring system fused to the ring containing Q₁ and Q₂, where the 5-12 membered ring is partially unsaturated or aromatic and optionally contains one or two of oxygen,
S(O)ₘ, nitrogen, or —NR₃₃ where R₃₃ is hydrogen or C₁-C₆ alkyl;
each R_{C} independently is halogen, cyano, nitro, or —R_{N};
each R_{N} is independently —R_{N′}, —C(O)R_{N′}, —C(O)OR_{N′}, —C(O)N(R_{N′})₂, —S(O)R_{N′}, or —S(O)₂R_{N′} wherein
each R_{N′} is independently hydrogen, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl, wherein
each R_{N′} is optionally substituted with from 1 to 4 R groups;
each R_{O} is independently —R_{N′}, —C(O)R_{N′}, —C(O)OR_{N′}, or —C(O)N(R_{N′})₂; and
each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆)alkylamino, carboxy, carboxamide, C₃-C₇ cycloalkyl, heterocycloalkyl, aryl, or heteroaryl,
the process comprising reacting a nitrile of formula F1 with a compound of formula F2 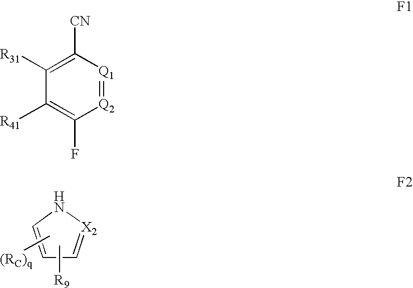
in the presence of a strong base.

105. A process for preparing a compound of formula F4 or F8 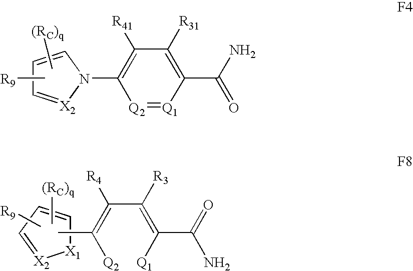
where
Q₁ and Q₂ are independently N or CR_{Q}, wherein
each R_{Q} is independently hydrogen, halogen, —O(R_{O}), —N(R_{N})₂, C₁-C₆ alkyl, C₁-C₆ haloalkyl, C₃-C₇ cycloalkyl, aryl, or heteroaryl, or R₂₁, wherein
each R_{Q} is optionally substituted with from 1 to 4 R groups; and
R₂₁ is cyano, —C(O)OH, —C(O)—O(C₁-C₆alkyl), or —C(X)N(R₁₁₁)₂, wherein
each R₁₁₁ is independently H, hydroxy, C₁-C₆ alkyl, C₂-C₆ alkenyl, C₂-C₆ alkynyl, heteroaryl, aryl, C₃-C₈ cycloalkyl, heterocycloalkyl,
wherein each R₁₁₁ is optionally substituted with from 1 to 4 R groups;
or both R₁₁₁ taken together with the nitrogen to which they are attached, form a heterocycloalkyl; and
X is ═O, ═S, ═NH, ═NOH, ═N—NH₂, ═N—NH-aryl, ═N—═NH—(C₁-C₆ alkyl), or ═N—(C₁-C₆ alkoxy)
wherein
m is 0, 1, or 2;
X₂ is C, N, O, or S;
R₉ is —C₁-C₆ alkoxy or a group of the formula, 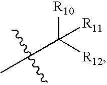
wherein
R₁₀ is C₁-C₆ alkyl or halo(C₁-C₆)alkyl, either optionally substituted with 1 or 2 R₁₀₁ groups, wherein
each R₁₀₁ is independently halogen, nitro, cyano, C₁-C₆ alkyl, halo(C₁-C₆)alkyl, —OR_{O}, —N(R_{N})₂, —S(O)ₘR_{N′}, or —C(O)R_{N′};
R₁₁ is H; and
R₁₂ is H or —OR_{O};
or R₁₁ and R₁₂ together are R₇; and
R₇ is O, S, NH, N—OH, N—NH₂, N—NHR₂₂, N—NH—(C₁-C₆ alkyl), N—O—(C₀-C₆)alkyl-R₂₂, or N—(C₁-C₆ alkoxy optionally substituted with carboxy);
each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, wherein
each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
each R₂₂ is optionally fused to a C₆-C₁₀ aryl group, C₅-C₈ saturated cyclic group, or a C₅-C₁₀ heterocycloalkyl group; and
R₃₁ and R₄₁ are independently (a) H, (b) halo, or (c) a C₁-C₁₅ alkyl group where up to six of the carbon atoms in said alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein each (c) is optionally substituted with —R_{C}, —OR₁₅, —SR₁₅, —N(R₁₅)₂, or —R₂₂, wherein
each R₁₅ is independently —H, (C₁-C₁₀)alkyl, (C₁-C₁₀) haloalkyl, (C₂-C₆)alkenyl, (C₂-C₆)alkynyl, or (C₁-C₁₀)alkyl-Z, wherein
Z is —OR_{O} or —N(R₃₀)₂, wherein
each R₃₀ is independently —H or C₁-C₆ alkyl;
or N(R₃₀)₂ represents pyrrolidinyl, piperidinyl, piperazinyl, azepanyl, 1,3- or 1,4-diazepanyl, or morpholinyl, each of which is optionally substituted with R;
or R₃₁ and R₄₁ together with the atoms to which they are attached form a 5-12 membered mono-, bi-, or tricyclic ring system fused to the ring containing Q₁ and Q₂, where the 5-12 membered ring is partially unsaturated or aromatic and optionally contains one or two of oxygen, S(O)ₘ, nitrogen, or —NR₃₃ where R₃₃ is hydrogen or C₁-C₆ alkyl;
each R_{C} independently is halogen, cyano, nitro, or —R_{N};
each R_{N} is independently —R_{N′}, —C(O)R_{N′}, —C(O)OR_{N′}, —C(O)N(R_{N′})₂, —S(O)R_{N′}, or —S(O)₂R_{N′} wherein
each R_{N′} is independently hydrogen, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl, wherein
each R_{N′} is optionally substituted with from 1 to 4 R groups;
each R_{O} is independently —R_{N′}, —C(O)R_{N′}, —C(O)OR_{N′}, or —C(O)N(R_{N′})₂; and
each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆)alkylamino, carboxy, carboxamide, C₃-C₇ cycloalkyl, heterocycloalkyl, aryl, or heteroaryl,
the process comprising oxidizing the nitrile group of a nitrile of formula F3 or F5 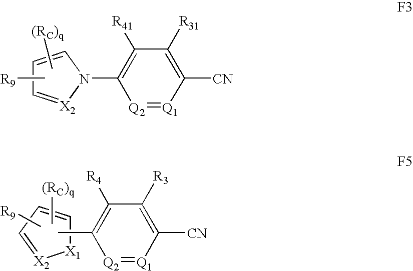
to the corresponding amide.

106. A process for preparing a compound of the formula F5 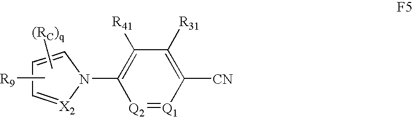
where
Q₁ and Q₂ are independently N or CR_{Q}, wherein
each R_{Q} is independently hydrogen, halogen, —O(R_{O}), —N(R_{N})₂, C₁-C₆ alkyl, C₁-C₆ haloalkyl, C₃-C₇ cycloalkyl, aryl, or heteroaryl, or R₂₁, wherein
each R_{Q} is optionally substituted with from 1 to 4 R groups; and
R₂₁ is cyano, —C(O)OH, —C(O)—O(C₁-C₆alkyl), or —C(X)N(R₁₁₁)₂, wherein
each R₁₁₁ is independently H, hydroxy, C₁-C₆ alkyl, C₂-C₆ alkenyl, C₂-C₆ alkynyl, heteroaryl, aryl, C₃-C₈ cycloalkyl, heterocycloalkyl,
wherein each R₁₁₁ is optionally substituted with from 1 to 4 R groups;
or both R₁₁₁ taken together with the nitrogen to which they are attached, form a heterocycloalkyl; and
X is ═O, ═S, ═NH, ═NOH, ═N—NH₂, ═N—NH-aryl, ═N—═NH—(C₁-C₆ alkyl), or ═N—(C₁-C₆ alkoxy);
wherein
m is 0, 1, or 2;
X₂ is C, N, O, or S;
R₉ is —C₁-C₆ alkoxy or a group of the formula, 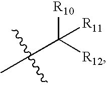
wherein
R₁₀ is C₁-C₆ alkyl or halo(C₁-C₆)alkyl, either optionally substituted with 1 or 2 R₁₀₁ groups, wherein
each R₁₀₁ is independently halogen, nitro, cyano, C₁-C₆ alkyl, halo(C₁-C₆)alkyl, —OR_{O}, —N(R_{N})₂, —S(O)ₘR_{N′}, or —C(O)R_{N′};
R₁₁ is H; and
R₁₂ is H or —OR_{O};
or R₁₁ and R₁₂ together are R₇; and
R₇ is O, S, NH, N—OH, N—NH₂, N—NHR₂₂, N—NH—(C₁-C₆ alkyl), N—O—(C₀-C₆)alkyl-R₂₂, or N—(C₁-C₆ alkoxy optionally substituted with carboxy);
each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, wherein
each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
each R₂₂ is optionally fused to a C₆-C₁₀ aryl group, C₅-C₈ saturated cyclic group, or a C₅-C₁₀ heterocycloalkyl group; and
R₃₁ and R₄₁ are independently (a) H, (b) halo, or (c) a C₁-C₁₅ alkyl group where up to six of the carbon atoms in said alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein each (c) is optionally substituted with —R_{C}, —OR₁₅, —SR₁₅, —N(R₁₅)₂, or —R₂₂, wherein
each R₁₅ is independently —H, (C₁-C₁₀)alkyl, (C₁-C₁₀) haloalkyl, (C₂-C₆)alkenyl, (C₂-C₆)alkynyl, or (C₁-C₁₀)alkyl-Z, wherein
Z is —OR_{O} or —N(R₃₀)₂, wherein
each R₃₀ is independently —H or C₁-C₆ alkyl;
or N(R₃₀)₂ represents pyrrolidinyl, piperidinyl, piperazinyl, azepanyl, 1,3- or 1,4-diazepanyl, or morpholinyl, each of which is optionally substituted with R;
or R₃₁ and R₄₁ together with the atoms to which they are attached form a 5-12 membered mono-, bi-, or tricyclic ring system fused to the ring containing Q₁ and Q₂, where the 5-12 membered ring is partially unsaturated or aromatic and optionally contains one or two of oxygen, S(O)ₘ, nitrogen, or —NR₃₃ where R₃₃ is hydrogen or C₁-C₆ alkyl;
each R_{C} independently is halogen, cyano, nitro, or —R_{N};
each R_{N} is independently —R_{N′}, —C(O)R_{N′}, —C(O)OR_{N′}, —C(O)N(R_{N′})₂, —S(O)R_{N′}, or —S(O)₂R_{N′} wherein
each R_{N′} is independently hydrogen, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl, wherein
each R_{N′} is optionally substituted with from 1 to 4 R groups;
each R_{O} is independently —R_{N′}, —C(O)R_{N′}, —C(O)OR_{N′}, or —C(O)N(R_{N′})₂; and
each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆)alkylamino, carboxy, carboxamide, C₃-C₇ cycloalkyl, heterocycloalkyl, aryl, or heteroaryl,
the process comprising treating a nitrile of formula F6 with a boronic acid of formula F7 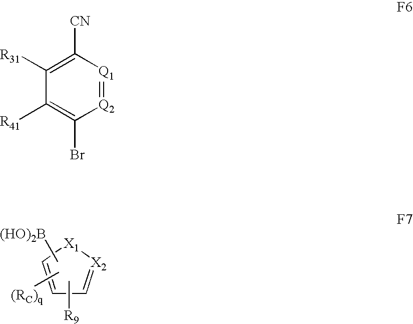
in the presence of a base and a catalyst.

107. A process for preparing a compound of the formula F9 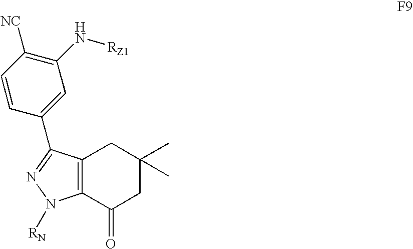
where R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S(O)ₘ—(C₁-C₆)alkyl, —S(O)ₘ-aryl, —SO₂NH₂, —SO₂NH—(C₁-C₆)alkyl, —SO₂NH-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₂, where each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, where
each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
each R₂₂ is optionally fused to a C₆-C₁₀ aryl group, C₅-C₈ saturated cyclic group, or a C₅-C₁₀ heterocycloalkyl group;
each m is independently 0, 1, or 2; and
each R_{N} is independently —R_{N′}, —C(O)R_{N′}, —C(O)OR_{N′}, —C(O)N(R_{N′})₂, —S(O)R_{N′}, or —S(O)₂R_{N′} wherein
each R_{N′} is independently hydrogen, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl, wherein
each R_{N′} is optionally substituted with from 1 to 4 R groups; and
each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆) alkylamino, carboxy, carboxamide, C₃-C₇ cycloalkyl, heterocycloalkyl, aryl, or heteroaryl;
comprising forming a reaction mixture of a compound of formula F10 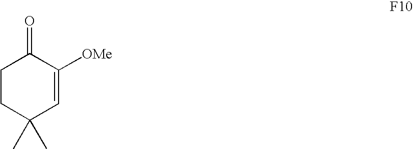
and a strong base in a solvent and
adding to the reaction mixture a nitrile of formula F11 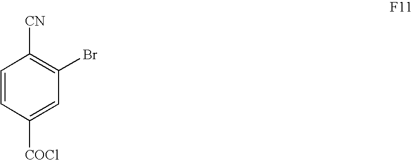
to yield a diketone of formula F12 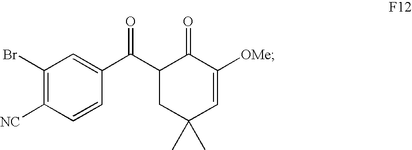
reacting the diketone with R_{N}—NHNH₂ to form an indazole of formula F13 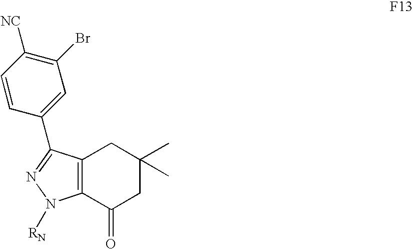
reacting the indazole F13 with an amine of the formula H₂NR_{Z1}, to provide a 2-amino benzonitrile of formula F14

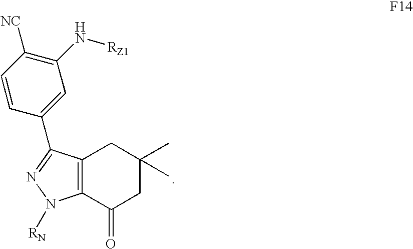

108. A process for preparing a compound of formula F15 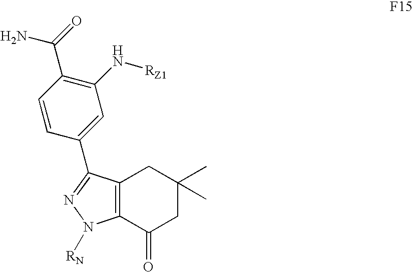
where R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S(O)ₘ—(C₁-C₆)alkyl, —S(O)ₘ-aryl, —SO₂NH₂, —SO₂NH—(C₁-C₆)alkyl, —SO₂NH-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₂, where each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, where
each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
each R₂₂ is optionally fused to a C₆-C₁₀ aryl group, C₅-C₈ saturated cyclic group, or a C₅-C₁₀ heterocycloalkyl group;
each m is independently 0, 1, or 2; and
each R_{N} is independently —R_{N′}, —C(O)R_{N′}, —C(O)OR_{N′}, —C(O)N(R_{N′})₂, —S(O)R_{N′}, or —S(O)₂R_{N′} wherein
each R_{N′} is independently hydrogen, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl, wherein
each R_{N′} is optionally substituted with from 1 to 4 R groups; and
each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆) alkylamino, carboxy, carboxamide, C₃-C₇ cycloalkyl, heterocycloalkyl, aryl, or heteroaryl;
comprising oxidizing the nitrile group of a nitrile of a compound of Formula F14.

109. A process for preparing a nitrile of formula F21 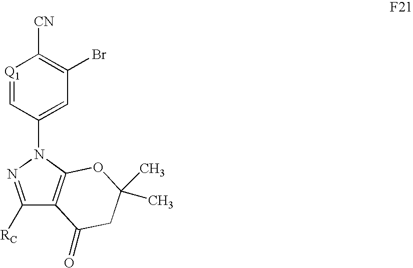
where
Q₁ is N or CR_{Q}, wherein
R_{Q} is hydrogen, halogen, —O(R_{O}), —N(R_{N})₂, C₁-C₆ alkyl, C₁-C₆ haloalkyl, C₃-C₇ cycloalkyl, aryl, or heteroaryl, or R₂₁, and
R_{Q} is optionally substituted with from 1 to 4 R groups; and
R₂₁ is —C(O)OH, —C(O)—O(C₁-C₆alkyl), or —C(X)N(R₁₁₁)₂, wherein
each R₁₁₁ is independently H, hydroxy, C₁-C₆ alkyl, C₂-C₆ alkenyl, C₂-C₆ alkynyl, heteroaryl, aryl, C₃-C₈ cycloalkyl, heterocycloalkyl,
wherein each R₁₁₁ is optionally substituted with from 1 to 4 R groups;
or both R₁₁₁ taken together with the nitrogen to which they are attached, form a heterocycloalkyl; and
X is ═O, ═S, ═NH, ═NOH, ═N—NH₂, ═N—NH-aryl, ═N—═NH—(C₁-C₆ alkyl), or ═N—(C₁-C₆ alkoxy);
each R_{O} is independently —R_{N′}, —C(O)R_{N′}, —C(O)OR_{N′}, or —C(O)N(R_{N′})₂;
each R_{N} is independently —R_{N′}, —C(O)R_{N′}, —C(O)OR_{N′}, —C(O)N(R_{N′})₂, —S(O)R_{N′}, or —S(O)₂R_{N′} wherein
each R_{N′} is independently hydrogen, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl, wherein
each R_{N′} is optionally substituted with from 1 to 4 R groups; and
each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆)alkylamino, carboxy, carboxamide, C₃-C₇ cycloalkyl, heterocycloalkyl, aryl, or heteroaryl;
the process comprising
treating a nitrile of formula F17 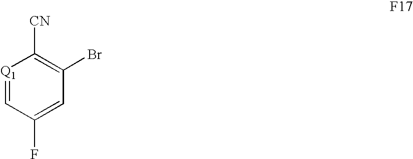
with hydrazine to form a 4-hyrazino-arylnitrile of formula F18 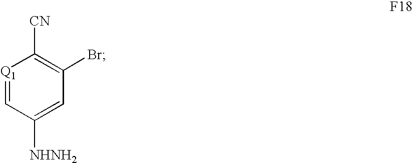
treating F18 with 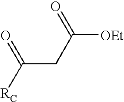
to generate a compound of formula F19 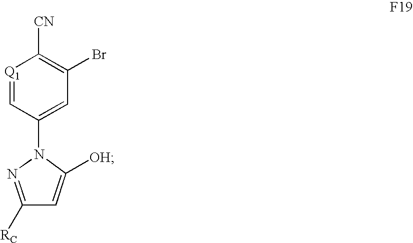
treating F19 with magnesium ethoxide followed by 3-methyl-but-2-enoyl chloride to yield a pyrazole of formula F20 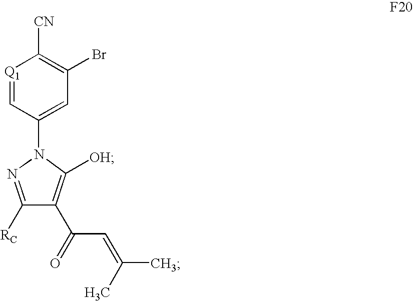
cyclizing F20 under acid conditions to form a dihydropyranopyrazole of formula F21

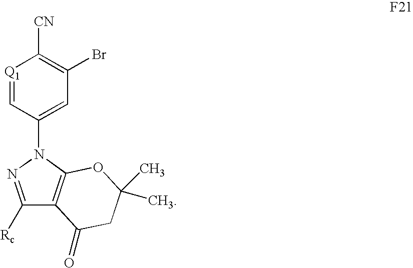

110. A process for preparing a compound of formula F21 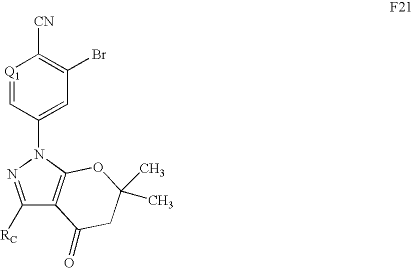
where
Q₁ is N or CR_{Q}, wherein
R_{Q} is hydrogen, halogen, —O(R_{O}), —N(R_{N})₂, C₁-C₆ alkyl, C₁-C₆ haloalkyl, C₃-C₇ cycloalkyl, aryl, or heteroaryl, or R₂₁, and
R_{Q} is optionally substituted with from 1 to 4 R groups; and
R₂₁ is —C(O)OH, —C(O)—O(C₁-C₆alkyl), or —C(X)N(R₁₁₁)₂ wherein
each R₁₁₁ is independently H, hydroxy, C₁-C₆ alkyl, C₂-C₆ alkenyl, C₂-C₆ alkynyl, heteroaryl, aryl, C₃-C₈ cycloalkyl, heterocycloalkyl,
wherein each R₁₁₁ is optionally substituted with from 1 to 4 R groups;
or both R₁₁₁ taken together with the nitrogen to which they are attached, form a heterocycloalkyl; and
X is ═O, ═S, ═NH, ═NOH, ═N—NH₂, ═N—NH-aryl, ═N—═NH—(C₁-C₆ alkyl), or ═N—(C₁-C₆ alkoxy);
each R_{O} is independently —R_{N′}, —C(O)R_{N′}, —C(O)OR_{N′}, or —C(O)N(R_{N′})₂;
each R_{N} is independently —R_{N′}, —C(O)R_{N′}, —C(O)OR_{N′}, —C(O)N(R_{N′})₂, —S(O)R_{N′}, or —S(O)₂R_{N′} wherein
each R_{N′} is independently hydrogen, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl, wherein
each R_{N′} is optionally substituted with from 1 to 4 R groups; and
each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆)alkylamino, carboxy, carboxamide, C₃-C₇ cycloalkyl, heterocycloalkyl, aryl, or heteroaryl;
the process comprising
treating a hydrazinoarylnitrile of formula F18 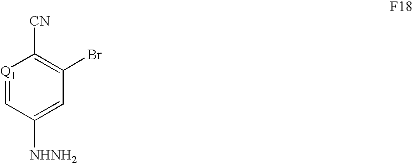
with a dehydroacetic acid salt to form a 4-hydroxypyranone of formula F22 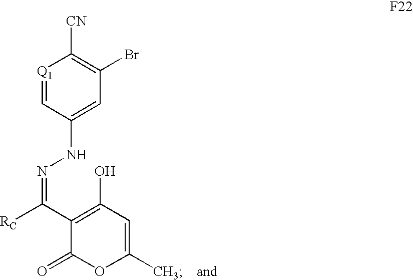
treating the pyranone F22 with acid.

111. A process for preparing a compound of formula F16 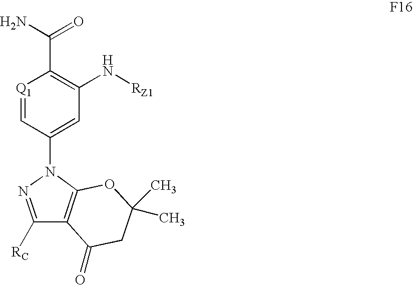
where
Q₁ is N or CR_{Q}, wherein
R_{Q} is hydrogen, halogen, —O(R_{O}), —N(R_{N})₂, C₁-C₆ alkyl, C₁-C₆ haloalkyl, C₃-C₇ cycloalkyl, aryl, or heteroaryl, or R₂₁, and
R_{Q} is optionally substituted with from 1 to 4 R groups; and
R₂₁ is —C(O)OH, —C(O)—O(C₁-C₆alkyl), or —C(X)N(R₁₁₁)₂, wherein
each R₁₁₁ is independently H, hydroxy, C₁-C₆ alkyl, C₂-C₆ alkenyl, C₂-C₆ alkynyl, heteroaryl, aryl, C₃-C₈ cycloalkyl, heterocycloalkyl,
wherein each R₁₁₁ is optionally substituted with from 1 to 4 R groups;
or both R₁₁₁ taken together with the nitrogen to which they are attached, form a heterocycloalkyl; and
X is ═O, ═S, ═NH, ═NOH, ═N—NH₂, ═N—NH-aryl, ═N≡NH—(C₁-C₆ alkyl), or ═N—(C₁-C₆ alkoxy);
each R_{O} is independently —R_{N′}, —C(O)R_{N′}, —C(O)OR_{N′}, or —C(O)N(R_{N′})₂;
each R_{N} is independently —R_{N′}, —C(O)R_{N′}, —C(O)OR_{N′}, —C(O)N(R_{N′})₂, —S(O)R_{N′}, or —S(O)₂R_{N′} wherein
each R_{N′} is independently hydrogen, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl, wherein
each R_{N′} is optionally substituted with from 1 to 4 R groups; and
each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆)alkylamino, carboxy, carboxamide, C₃-C₇ cycloalkyl, heterocycloalkyl, aryl, or heteroaryl;
where R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S(O)ₘ₋₁ (C₁-C₆)alkyl, —S(O)ₘ-aryl, —SO₂NH₂, —SO₂NH—(C₁-C₆)alkyl, —SO₂NH-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₂, where each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, where
each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
each R₂₂ is optionally fused to a C₆-C₁₀ aryl group, C₅-C₈ saturated cyclic group, or a C₅-C₁₀ heterocycloalkyl group; and
each m is independently 0, 1, or 2;
comprising treating a dihydropyranopyrazole of formula F21 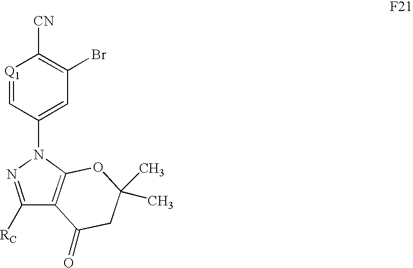
with an amine of the formula H₂NR_{Z1} to provide a 2-aminoarylnitrile of formula F23 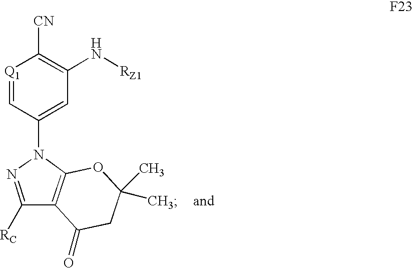
oxidizing the nitrile group of formula F23.

112. A process for preparing a nitrile of formula F24 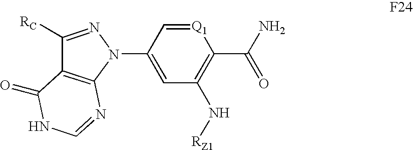
where
Q₁ is N or CR_{Q}, wherein
R_{Q} is hydrogen, halogen, —O(R_{O}), —N(R_{N})₂, C₁-C₆ alkyl, C₁-C₆ haloalkyl, C₃-C₇ cycloalkyl, aryl, or heteroaryl, or R₂₁, and
R_{Q} is optionally substituted with from 1 to 4 R groups; and
R₂₁ is —C(O)OH, —C(O)—O(C₁-C₆alkyl), or —C(X)N(R₁₁₁)₂, wherein
each R₁₁₁ is independently H, hydroxy, C₁-C₆ alkyl, C₂-C₆ alkenyl, C₂-C₆ alkynyl, heteroaryl, aryl, C₃-C₈ cycloalkyl, heterocycloalkyl,
wherein each R₁₁₁ is optionally substituted with from 1 to 4 R groups;
or both R₁₁₁ taken together with the nitrogen to which they are attached, form a heterocycloalkyl; and
X is ═O, ═S, ═NH, ═NOH, ═N—NH₂, ═N—NH-aryl, ═N—═NH—(C₁-C₆ alkyl), or ═N—(C₁-C₆ alkoxy);
each R_{O} is independently —R_{N′}, —C(O)R_{N′}, —C(O)OR_{N′}, or —C(O)N(R_{N′})₂;
each R_{N} is independently —R_{N′}, —C(O)R_{N′}, —C(O)OR_{N′}, —C(O)N(R_{N′})₂, —S(O)R_{N′}, or —S(O)₂R_{N′} wherein
each R_{N′} is independently hydrogen, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl, wherein
each R_{N′} is optionally substituted with from 1 to 4 R groups; and
each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆)alkylamino, carboxy, carboxamide, C₃-C₇ cycloalkyl, heterocycloalkyl, aryl, or heteroaryl;
R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S(O)ₘ—(C₁-C₆)alkyl, —S(O)ₘ-aryl, —SO₂NH₂, —SO₂NH—(C₁-C₆)alkyl, —SO₂NH-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₂, where each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, where
each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
each R₂₂ is optionally fused to a C₆-C₁₀ aryl group, C₅-C₈ saturated cyclic group, or a C₅-C₁₀ heterocycloalkyl group; and
each m is 0, 1, or 2;
the process comprising
treating a nitrile of formula F25 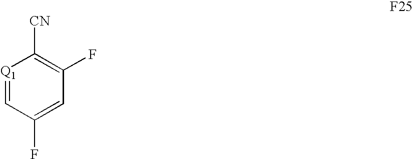
with an amine of the formula H₂NR_{Z1} to provide a 4-fluoroarylnitrile of formula F26 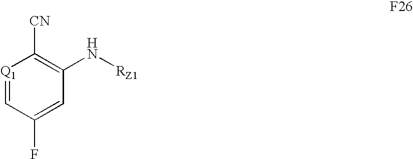
treating the nitrile of formula F26 with hydrazine to form a compound of formula F27 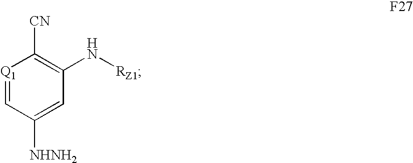
treating compound of formula F27 with 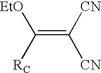
to yield a pyrazole of formula F28 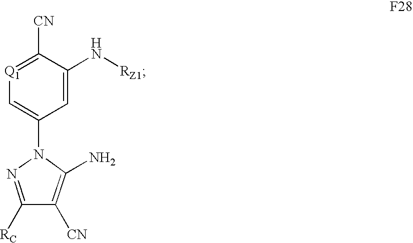
treating the pyrazole of formula F28 with formic acid to yield a compound of formula F29 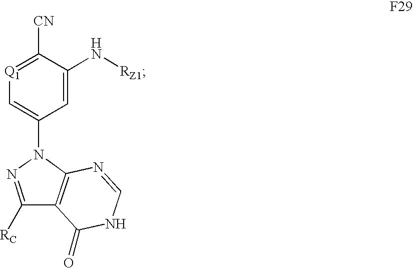
and oxidizing the nitrile of the compound of formula F29.

113. A process for preparing a compound of formula F30 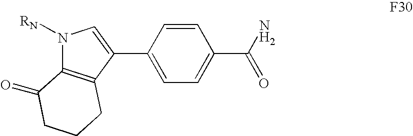
where
R_{N} is independently —R_{N′}, —C(O)R_{N′}, —C(O)OR_{N′}, —C(O)N(R_{N′})₂, —S(O)R_{N′}, or —S(O)₂R_{N′} wherein
each R_{N′} is independently hydrogen, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl, wherein
each R_{N′} is optionally substituted with from 1 to 4 R groups; and
each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆)alkylamino, carboxy, carboxamide, C₃-C₇ cycloalkyl, heterocycloalkyl, aryl, or heteroaryl;
the process comprising
reacting a compound of formula F31 with a compound of formula F32 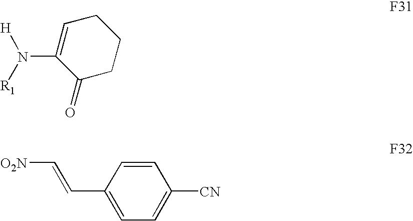
to form a benzonitrile of formula F33 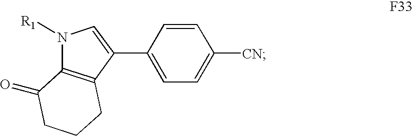
and oxidizing the nitrile of the benzonitrile of formula F33.

114. A process for preparing a compound of formula F34 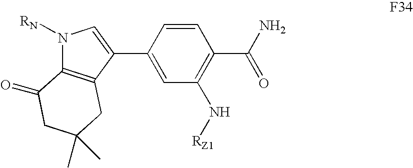
where
R_{N} is independently —R_{N′}, —C(O)R_{N′}, —C(O)OR_{N′}, —C(O)N(R_{N′})₂, —S(O)R_{N′}, or —S(O)₂R_{N′} wherein
each R_{N′} is independently hydrogen, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl, wherein
each R_{N′} is optionally substituted with from 1 to 4 R groups; and
each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆)alkylamino, carboxy, carboxamide, C₃-C₇ cycloalkyl, heterocycloalkyl, aryl, or heteroaryl;
R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S(O)ₘ—(C₁-C₆)alkyl, —S(O)ₘ-aryl, —SO₂NH₂, —SO₂NH—(C₁-C₆)alkyl, —SO₂NH-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₂, where each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, where
each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
each R₂₂ is optionally fused to a C₆-C₁₀ aryl group, C₅-C₈ saturated cyclic group, or a C₅-C₁₀ heterocycloalkyl group; and
each m is 0, 1, or 2;
the process comprising
reacting a compound of formula F35 with a compound of formula F36 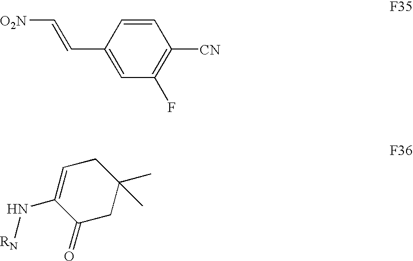
to form a benzonitrile of formula F37 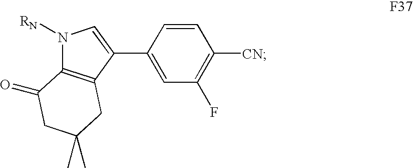
reacting the benzonitrile of formula F37 with an amine of the formula H₂NR_{Z1} to provide a 4-indol-3-ylbenzonitrile of formula F38; 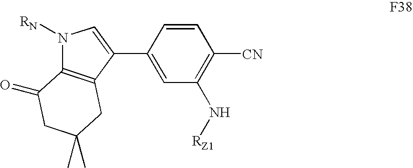
and oxidizing the nitrile of the indolylbenzonitrile.

115. A process for preparing a compound of formula F39 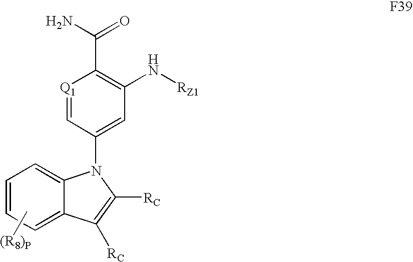
wherein
Q₁ is N or CR_{Q}, wherein
R_{Q} is hydrogen, halogen, —O(R_{O}), —N(R_{N})₂, C₁-C₆ alkyl, C₁-C₆ haloalkyl, C₃-C₇ cycloalkyl, aryl, or heteroaryl, or R₂₁, and
R_{Q} is optionally substituted with from 1 to 4 R groups; and
R₂₁ is —C(O)OH, —C(O)—O(C₁-C₆alkyl), or —C(X)N(R₁₁₁)₂, wherein
each R₁₁₁ is independently H, hydroxy, C₁-C₆ alkyl, C₂-C₆ alkenyl, C₂-C₆ alkynyl, heteroaryl, aryl, C₃-C₈ cycloalkyl, heterocycloalkyl,
wherein each R₁₁₁ is optionally substituted with from 1 to 4 R groups;
or both R₁₁₁ taken together with the nitrogen to which they are attached, form a heterocycloalkyl; and
X is ═O, ═S, ═NH, ═NOH, ═N—NH₂, ═N—NH-aryl, ═N—═NH—(C₁-C₆ alkyl), or ═N—(C₁-C₆ alkoxy);
each R_{C} independently is halogen, cyano, nitro, or —R_{N};
each R₁₁ is independently —H, —R₂₂, C₁-C₆ alkyl, or halo(C₁-C₆)alkyl,
wherein each R₈₁ is optionally substituted with 1-2 groups which are independently R_{C}, —OR_{O}, —SR_{O}, or —N(R_{N})₂;
each R_{N} is independently —R_{N′}, —C(O)R_{N′}, —C(O)OR_{N′}, —C(O)N(R_{N′})₂, —S(O)R_{N′}, or —S(O)₂R_{N′} wherein
each R_{N′} is independently hydrogen, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀) alkyl, heterocycloalkyl, heterocycloalkyl(C₁-C₁₀) alkyl, aryl, aryl(C₁-C₁₀) alkyl, heteroaryl, or heteroaryl(C₁-C₁₀)alkyl, wherein
each R_{N′} is optionally substituted with from 1 to 4 R groups;
each R_{O} is independently —R_{N′}, —C(O)R_{N′}, —C(O)OR_{N′}, or —C(O)N(R_{N′})₂;
each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆)alkylamino, carboxy, carboxamide, C₃-C₇ cycloalkyl, heterocycloalkyl, aryl, or heteroaryl; and
R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S(O)ₘ—(C₁-C₆)alkyl, —S(O)ₘ-aryl, —SO₂NH₂, —SO₂NH—(C₁-C₆)alkyl, —SO₂NH-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₂, where
each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, where
each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
each R₂₂ is optionally fused to a C₆-C₁₀ aryl group, C₅-C₈ saturated cyclic group, or a C₅-C₁₀ heterocycloalkyl group; and
each m is 0, 1, or 2;
the process comprising
reacting a compound of formula F35 with a indole of formula F36 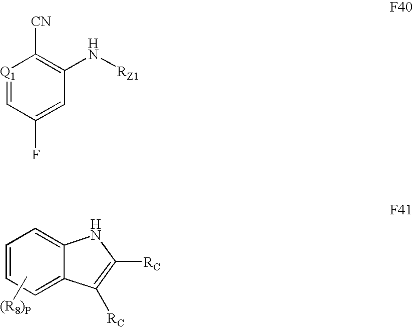
to form a compound of formula F42 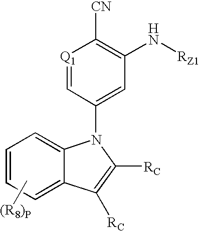
and oxidizing the nitrile of the compound of formula F42.

116. A compound of the formula: 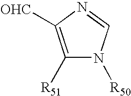
where
R₅₀ is a nitrogen protecting group; and
R₅₁ is C₁-C₁₀ alkyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl.

117. A compound according to claim 116, where R₅₀ is triphenylmethyl.

118. A compound according to claim 116, where R₅₁ is C₁-C₆ alkyl, C₁-C₆ haloalkyl, C₃-C₇ cycloalkyl, or C₃-C₇ cycloalkyl(C₁-C₁₀)alkyl.

119. A compound according to claim 117, where R₅₁ is C₁-C₆ alkyl, C₁-C₆ haloalkyl, C₃-C₇ cycloalkyl, or C₃-C₇ cycloalkyl(C₁-C₁₀)alkyl.

120. A compound according to claim 116, where R₅₁ is C₁-C₃ alkyl, C₁-C₃ haloalkyl, C₃-C₅ cycloalkyl, or C₃-C₅ cycloalkyl(C₁-C₃)alkyl.

121. A compound according to claim 117, where R₅₁ is C₁-C₃ alkyl, C₁-C₃ haloalkyl, C₃-C₅ cycloalkyl, or C₃-C₅ cycloalkyl(C₁-C₃)alkyl.

122. A compound of the formula 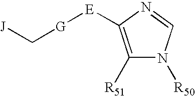
wherein
J is a protected hydroxy group;
E represents —CH(OH)— or —C(O)—;
G is C₂-alkynylene or C₂-alkylene;
R₅₀ is a nitrogen protecting group; and
R₅₁ is C₁-C₁₀ alkyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl.

123. A compound according to claim 122, where J is THP—O— and
R₅₀ is triphenylmethyl.

124. A compound according to claim 122, where J is THP—O— and R₅₁ is C₁-C₆ alkyl, C₁-C₆ haloalkyl, C₃-C₇ cycloalkyl, or C₃-C₇ cycloalkyl(C₁-C₁₀)alkyl.

125. A compound according to claim 123, where R₅₁ is C₁-C₆ alkyl, C₁-C₆ haloalkyl, C₃-C₇ cycloalkyl, or C₃-C₇ cycloalkyl(C₁-C₁₀)alkyl.

126. A compound according to claim 122, where J is THP—O— and R₅₁ is C₁-C₃ alkyl, C₁-C₃ haloalkyl, C₃-C₅ cycloalkyl, or C₃-C₅ cycloalkyl(C₁-C₃)alkyl.

127. A compound according to claim 123, where R₅₁ is C₁-C₃ alkyl, C₁-C₃ haloalkyl, C₃-C₅ cycloalkyl, or C₃-C₅ cycloalkyl(C₁-C₃)alkyl.

128. A compound according to claim 127, where E represents —CH(OH)—.

129. A compound according to claim 127, where E represents —C(O)—.

130. A compound according to claim 127, where G represents C₂-alkynylene.

131. A compound according to claim 127, where G represents C₂₋-alkylene.

132. A compound of the formula: 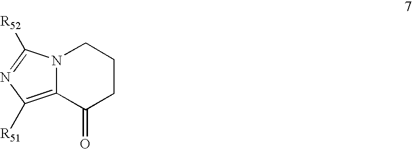
wherein
R₅₁ is C₁-C₁₀ alkyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀)alkyl, aryl, aryl(C₁-C₁₀)alkyl, heteroaryl, or heteroaryl(C₁-C₁₀)alkyl;
R₅₂ is
halogen or
phenyl that is
substituted with one fluoro,
optionally substituted with one of cyano, amino(C₁-C₆)alkyl, mono- or di(C₁-C₆)alkylamino(C₁-C₃)alkyl, —C(O)NR₅₃R₅₄ where R₅₃ and R₅₄ independently represent hydrogen or C₁-C₆ alkyl, and
optionally substituted with one of C₁-C₆ alkyl, halo(C₁-C₆)alkyl, C₃-C₇cycloalkyl, bromo, iodo, chloro, nitro, or C₃-C₇ cycloalkyl(C₁-C₃)alkyl.

133. A compound according to claim 132, where R₅₂ is hydrogen and R₅₁ is C₁-C₃ alkyl, C₁-C₃ haloalkyl, C₃-C₅ cycloalkyl, or C₃-C₅ cycloalkyl(C₁-C₃)alkyl.

134. A compound according to claim 132, where R₅₂ is halogen and R₅₁ is C₁-C₃ alkyl, C₁-C₃ haloalkyl, C₃-C₅ cycloalkyl, or C₃-C₅ cycloalkyl(C₁-C₃)alkyl.

135. A compound according to claim 132, where R₅₂ is bromo or chloro and R₅₁ is C₁-C₃ alkyl, C₁-C₃ haloalkyl, C₃-C₅ cycloalkyl, or C₃-C₅ cycloalkyl(C₁-C₃)alkyl.

136. A compound according to claim 132, where R₅₂ is phenyl substituted with fluoro and cyano, and R₅₁ is C₁-C₃ alkyl, C₁-C₃ haloalkyl, C₃-C₅ cycloalkyl, or C₃-C₅ cycloalkyl(C₁-C₃)alkyl.

137. A compound according to claim 132, where R₅₂ is 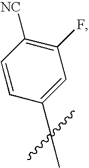
and
R₅₁ is C₁-C₃ alkyl, C₁-C₃ haloalkyl, C₃-C₅ cycloalkyl, or C₃-C₅ cycloalkyl(C₁-C₃)alkyl.

138. A compound of the formula 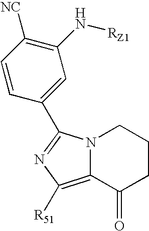
where
R₅₁ is C₁-C₁₀ alkyl, C₁-C₁₀ haloalkyl, C₃-C₇ cycloalkyl, C₃-C₇ cycloalkyl(C₁-C₁₀)alkyl, heterocycloalkyl, heterocycloalkyl(C₁-C₁₀) alkyl, aryl, aryl(C₁-C₁₀) alkyl, heteroaryl, or heteroaryl(C₁-C₁₀)alkyl; and
R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S(O)ₘ—(C₁-C₆)alkyl, —S(O)ₘ-aryl, —SO₂NH₂, —SO₂NH—(C₁-C₆)alkyl, —SO₂NH-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₂, where each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, where
each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
each R₂₂ is optionally fused to a C₆-C₁₀ aryl group, C₅-C₈ saturated cyclic group, or a C₅-C₁₀ heterocycloalkyl group.

139. A compound according to claim 138, where R₅₁ is C₁-C₃ alkyl, C₁-C₃ haloalkyl, C₃-C₅ cycloalkyl, or C₃-C₅ cycloalkyl(C₁-C₃)alkyl.

140. A compound according to claim 138, where R_{Z1} is tetrahydro-pyran-4-yl, tetrahydro-furan-2-ylmethyl, 4-hydroxy-cyclohexyl, 1-methoxypropan-2-yl, phenyl, 2-methoxyethyl, or 2-hydroxycyclopentyl.

141. A compound of the formula: 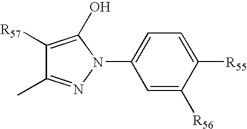
wherein
R₅₅ is cyano, amino(C₁-C₆)alkyl, mono- or di(C₁-C₆)alkylamino(C₁-C₃)alkyl, —C(O)NR₅₃R₅₄ where R₅₃ and R₅₄ independently represent hydrogen or C₁-C₆ alkyl;
R₅₆ is halogen; and
R₅₇ is hydrogen or 3-methylbut-2-enoyl.

142. A compound according to claim 141, where R₅₅ is cyano or —C(O)NR₅₃R₅₄.

143. A compound of the formula: 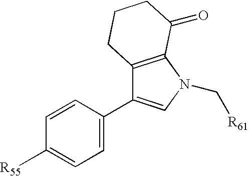
wherein
R₅₅ is cyano, amino(C₁-C₆)alkyl, mono- or di(C₁-C₆)alkylamino(C₁-C₃)alkyl, —C(O)NR₅₃R₅₄ where R₅₃ and R₅₄ independently represent hydrogen or C₁-C₆ alkyl; and
R₆₁ is phenyl substituted with 1-3 of hydroxy, C₁-C₆ alkoxy, C₁-C₆ alkyl, C₁-C₆ alkyl(C₁-C₆)alkoxy, or C₃-C₇ cycloalkyl(C₁-C₆)alkoxy.

144. A compound according to claim 143, where R₅₅ is cyano.

145. A compound according to claim 144, where R₆₁ is phenyl substituted with at least one of C₁-C₆ alkoxy.

146. A compound according to claim 144, where R₆₁ is phenyl substituted with at least two of C₁-C₆ alkoxy.

147. A compound of the formula: 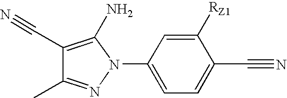
R_{Z1} is a C₁-C₁₄ alkyl group where up to five of the carbon atoms in the alkyl group are optionally replaced independently by R₂₂, carbonyl, ethenyl, ethynyl or a moiety selected from N, O, or S(O)ₘ, with the proviso that two O atoms, two S atoms, or an and S atom are not immediately adjacent each other,
wherein R_{Z1} is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S(O)ₘ—(C₁-C₆)alkyl, —S(O)ₘ-aryl, —SO₂NH₂, —SO₂NH—(C₁-C₆)alkyl, —SO₂NH-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₂, where each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, where
each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
each R₂₂ is optionally fused to a C₆-C₁₀ aryl group, C₅-C₈ saturated cyclic group, or a C₅-C₁₀ heterocycloalkyl group.

148. A compound according to claim 147, where R_{Z1} is tetrahydro-pyran-4-ylamino, tetrahydro-furan-2-ylmethylamino, 4-hydroxy-cyclohexylamino, phenylamino, or 2-hydroxycyclopentylamino.

149. A compound of the formula: 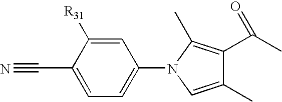
wherein
R₃₁ is (a) H, (b) halo, or (c) a C₁-C₁₅ alkyl group where up to six of the carbon atoms in said alkyl group are optionally replaced independently by R₂₂, carbonyl, ethenyl, ethynyl or a moiety selected from N, O, or S(O)ₘ, with the proviso that two O atoms, two S atoms, or an O and S atom are not immediately adjacent each other,
wherein each (c) is optionally substituted with —R_{C} —OR₁₅, —SR₁₅, —N(R₁₅)₂, or —R₂₂, wherein
each R₁₅ is independently —H, (C₁-C₁₀)alkyl, (C₁-C₁₀) haloalkyl, (C₂-C₆)alkenyl, (C₂-C₆)alkynyl, or (C₁-C₁₀)alkyl-Z, wherein
Z is —OR_{O} or —N(R₃₀)₂, wherein
each R₃₀ is independently —H or C₁-C₆ alkyl;
or N(R₃₀)₂ represents pyrrolidinyl, piperidinyl, piperazinyl, azepanyl, 1,3- or 1,4-diazepanyl, or morpholinyl, each of which is optionally substituted with R;
each R is independently halogen, cyano, nitro, C₁-C₆ alkyl, halo(C₁-C₆)alkyl, hydroxy, C₁-C₆ alkoxy, halo(C₁-C₆)alkoxy, amino, mono- or di-(C₁-C₆)alkylamino, carboxy, carboxamide, C₃-C₇ cycloalkyl, heterocycloalkyl, aryl, or heteroaryl;
each R₂₂ is independently (i) heteroaryl, (ii) aryl, (iii) saturated or unsaturated C₃-C₁₀ cycloalkyl, or (iv) saturated or unsaturated C₂-C₁₀ heterocycloalkyl, wherein
each R₂₂ is optionally substituted with 1 to 4 groups which are independently R, oxo, —SH, —S(O)ₘ—(C₁-C₆)alkyl, —S(O)ₘ-aryl, —SO₂NH₂, —SO₂NH—(C₁-C₆)alkyl, or —SO₂NH-aryl; and
each R₂₂ is optionally fused to a C₆-C₁₀ aryl group, C₅-C₈ saturated cyclic group, or a C₅-C₁₀ heterocycloalkyl group.

150. A compound according to claim 149, where R₃₁ is amino substituted with
C₃-C₇cycloalkyl substituted with amino, halogen, hydroxy, C₁-C₆ alkoxy, or nitro, or
a 4-7 membered heterocycloalkyl group optionally substituted with amino, halogen, hydroxy, C₁-C₆ alkoxy, or nitro.

151. A compound according to claim 149, wherein R₃₁ amino substituted with hydroxycyclohexyl or tetrahydropyranyl.
